(19) United States
(12) Patent Application Publication
(10) Pub. No.: US 2006/0234246 A1
(43) Pub. Date: Oct. 19, 2006

(54) Gene products differentially expressed in cancerous cells

(75) Inventors: Elizabeth M. Scott, Emeryville, CA (US); George Lamson, Emeryville, CA (US); Altaf Kassam, Emeryville, CA (US); Guozhong Zhang, Emeryville, CA (US); Doreen Sakamoto, Emeryville, CA (US); Pablo Dominguez Garcia, Emeryville, CA (US); Theresa May, Emeryville, CA (US); Giulia C. Kennedy, Emeryville, CA (US); Sanmao Kang, Emeryville, CA (US); Christoph Reinhard, Emeryville, CA (US); Ann Bennett Jefferson, Emeryville, CA (US)

Correspondence Address: BOZICEVIC, FIELD & FRANCIS LLP, Suite 200, 1900 University Avenue, East Palo Alto, CA, 94303, US

(73) Assignee: Chiron Corporation

(21) Appl. No.: 10/934,842

(22) Filed: Sep. 2, 2004

Related U.S. Application Data
(63) Continuation-in-part of application No. 10/165,835, filed on Jun. 6, 2002, now abandoned.
Application No. 10/165,835 is a continuation of application No. 09/490,818, filed on Jan. 25, 2000, now Pat. No. 6,429,302.
(63) Continuation-in-part of application No. 09/883,152, filed on Jun. 15, 2001, now abandoned.
(63) Continuation-in-part of application No. PCT/US03/15465, filed on May 16, 2003.
(60) Provisional application No. 60/118,302, filed on Feb. 2, 1999.
(60) Provisional application No. 60/211,835, filed on Jun. 15, 2000.
(60) Provisional application No. 60/445,222, filed on Feb. 4, 2003.
(60) Provisional application No. 60/381,533, filed on May 17, 2002.

(51) Int. Cl. C12Q 1/68; G01N 33/574; C07H 21/04; C12P 21/06; C07K 14/82; C07K 16/30

(52) U.S. Cl. 435/6; 435/7.23; 435/69.1; 435/320.1; 435/325; 530/350; 530/388.8; 536/23.5

(57) ABSTRACT

The present invention provides polynucleotides, as well as polypeptides encoded thereby, that are differentially expressed in cancer cells. These polynucleotides are useful in a variety of diagnostic and therapeutic methods. The present invention further provides methods of reducing growth of cancer cells. These methods are useful for treating cancer.

### FIELD OF THE INVENTION

The present invention relates to polynucleotides of human origin in substantially isolated form and gene products that are differentially expressed in cancer cells, and uses thereof.

### BACKGROUND OF THE INVENTION

Cancer, like many diseases, is not the result of a single, well-defined cause, but rather can be viewed as several diseases, each caused by different aberrations in informational pathways, that ultimately result in apparently similar pathologic phenotypes. Identification of polynucleotides that correspond to genes that are differentially expressed in cancerous, pre-cancerous, or low metastatic potential cells relative to normal cells of the same tissue type, provides the basis for diagnostic tools, facilitates drug discovery by providing for targets for candidate agents, and further serves to identify therapeutic targets for cancer therapies that are more tailored for the type of cancer to be treated.

Identification of differentially expressed gene products also furthers the understanding of the progression and nature of complex diseases such as cancer, and is key to identifying the genetic factors that are responsible for the phenotypes associated with development of, for example, the metastatic phenotype. Identification of gene products that are differentially expressed at various stages, and in various types of cancers, can both provide for early diagnostic tests, and further serve as therapeutic targets. Additionally, the product of a differentially expressed gene can be the basis for screening assays to identify chemotherapeutic agents that modulate its activity (e.g. its expression, biological activity, and the like).

Early disease diagnosis is of central importance to halting disease progression, and reducing morbidity. Analysis of a patient's tumor to identify the gene products that are differentially expressed, and administration of therapeutic agent(s) designed to modulate the activity of those differentially expressed gene products, provides the basis for more specific, rational cancer therapy that may result in diminished adverse side effects relative to conventional therapies. Furthermore, confirmation that a tumor poses less risk to the patient (e.g., that the tumor is benign) can avoid unnecessary therapies. In short, identification of genes and the encoded gene products that are differentially expressed in cancerous cells can provide the basis of therapeutics, diagnostics, prognostics, therametrics, and the like.

For example, breast cancer is a leading cause of death among women. One of the priorities in breast cancer research is the discovery of new biochemical markers that can be used for diagnosis, prognosis and monitoring of breast cancer. The prognostic usefulness of these markers depends on the ability of the marker to distinguish between patients with breast cancer who require aggressive therapeutic treatment and patients who should be monitored.

While the pathogenesis of breast cancer is unclear, transformation of non-tumorigenic breast epithelium to a malignant phenotype may be the result of genetic factors, especially in women under 30 (Miki, et al., Science, 266: 66-71, 1994). However, it is likely that other, non-genetic factors are also significant in the etiology of the disease. Regardless of its origin, breast cancer morbidity increases significantly if a lesion is not detected early in its progression. Thus, considerable effort has focused on the elucidation of early cellular events surrounding transformation in breast tissue. Such effort has led to the identification of several potential breast cancer markers.

Thus, the identification of new markers associated with cancer, for example, breast cancer, and the identification of genes involved in transforming cells into the cancerous phenotype, remains a significant goal in the management of this disease. In exemplary aspects, the invention described herein provides cancer diagnostics, prognostics, therametrics, and therapeutics based upon polynucleotides and/or their encoded gene products.

### SUMMARY OF THE INVENTION

The present invention provides methods and compositions useful in detection of cancerous cells, identification of agents that modulate the phenotype of cancerous cells, and identification of therapeutic targets for chemotherapy of cancerous cells. Cancerous, breast, colon and prostate cells are of particular interest in each of these aspects of the invention. More specifically, the invention provides polynucleotides in substantially isolated form, as well as polypeptides encoded thereby, that are differentially expressed in cancer cells. Also provided are antibodies that specifically bind the encoded polypeptides. These polynucleotides, polypeptides and antibodies are thus useful in a variety of diagnostic, therapeutic, and drug discovery methods. In some embodiments, a polynucleotide that is differentially expressed in cancer cells can be used in diagnostic assays to detect cancer cells. In other embodiments, a polynucleotide that is differentially expressed in cancer cells, and/or a polypeptide encoded thereby, is itself a target for therapeutic intervention.

Accordingly, the invention features an isolated polynucleotide comprising a nucleotide sequence having at least 90% sequence identity to an identifying sequence of any one of the sequences set forth herein or a degenerate variant thereof. In related aspects, the invention features recombinant host cells and vectors comprising the polynucleotides of the invention, as well as isolated polypeptides encoded by the polynucleotides of the invention and antibodies that specifically bind such polypeptides.

In other aspects, the invention provides a method for detecting a cancerous cell. In general, the method involves contacting a test sample obtained from a cell that is suspected of being a cancer cell with a probe for detecting a gene product differentially expressed in cancer. Many embodiments of the invention involve a gene identifiable by or comprising a sequence selected from the group consisting of SEQ ID NOS: 1, 3, 5, 7, 9, 11-13, 15, 16, 18, 20, 22, 24, 26, 27, 29 and 128-1618, contacting the probe and the gene product for a time sufficient for binding of the probe to the gene product; and comparing a level of binding of the probe to the sample with a level of probe binding to a control sample obtained from a control cell of known cancerous state. A modulated (i.e. increased or decreased) level of binding of the probe in the test cell sample relative to the level of binding in a control sample is indicative of the cancerous state of the test cell. In certain embodiments, the level of binding of the probe in the test cell sample, usually in relation to at least one control gene, is similar to binding of the probe to a cancerous cell sample. In certain other embodiments, the level of binding of the probe in the test cell sample, usually in relation to at least one control gene, is different, i.e. opposite, to binding of the probe to a non-cancerous cell sample. In specific embodiments, the probe is a polynucleotide probe and the gene product is nucleic acid. In other specific embodiments, the gene product is a polypeptide. In further embodiments, the gene product or the probe is immobilized on an array.

In another aspect, the invention provides a method for assessing the cancerous phenotype (e.g., metastasis, metastatic potential, aberrant cellular proliferation, and the like) of a cell comprising detecting expression of a gene product in a test cell sample, wherein the gene comprises or is identifiable using a sequence selected from the group consisting of SEQ ID NOS: 1, 3, 5, 7, 9, 11-13, 15, 16, 18, 20, 22, 24, 26, 27, 29 and 128-1618; and comparing a level of expression of the gene product in the test cell sample with a level of expression of the gene in a control cell sample. Comparison of the level of expression of the gene in the test cell sample relative to the level of expression in the control cell sample is indicative of the cancerous phenotype of the test cell sample. In specific embodiments, detection of gene expression is by detecting a level of an RNA transcript in the test cell sample. In other specific embodiments detection of expression of the gene is by detecting a level of a polypeptide in a test sample.

In another aspect, the invention provides a method for suppressing or inhibiting a cancerous phenotype of a cancerous cell, the method comprising introducing into a mammalian cell an expression modulatory agent (e.g. an antisense molecule, small molecule, antibody, neutralizing antibody, inhibitory RNA molecule, etc.) to inhibit expression of a gene identified by a sequence selected from the group consisting of SEQ ID NOS: 1, 3, 5, 7, 9, 11-13, 15, 16, 18, 20, 22, 24, 26, 27, 29 and 128-1618. Inhibition of expression of the gene inhibits development of a cancerous phenotype in the cell. In specific embodiments, the cancerous phenotype is metastasis, aberrant cellular proliferation relative to a normal cell, or loss of contact inhibition of cell growth. In the context of this invention “expression” of a gene is intended to encompass the expression of an activity of a gene product, and, as such, inhibiting expression of a gene includes inhibiting the activity of a product of the gene.

In another aspect, the invention provides a method for assessing the tumor burden of a subject, the method comprising detecting a level of a differentially expressed gene product in a test sample from a subject suspected of or having a tumor, the differentially expressed gene product identified by or comprising a sequence selected from the group consisting of SEQ ID NOS: 1, 3, 5, 7, 9, 11-13, 15, 16, 18, 20, 22, 24, 26, 27, 29 and 128-1618. Detection of the level of the gene product in the test sample is indicative of the tumor burden in the subject.

In another aspect, the invention provides a method for identifying agents that modulate (i.e. increase or decrease) the biological activity of a gene product differentially expressed in a cancerous cell, the method comprising contacting a candidate agent with a differentially expressed gene product, the differentially expressed gene product corresponding to a sequence selected from the group consisting of SEQ ID NOS: 1, 3, 5, 7, 9, 11-13, 15, 16, 18, 20, 22, 24, 26, 27, 29 and 128-1618; and detecting a modulation in a biological activity of the gene product relative to a level of biological activity of the gene product in the absence of the candidate agent. In specific embodiments, the detecting is by identifying an increase or decrease in expression of the differentially expressed gene product. In other specific embodiments, the gene product is mRNA or cDNA prepared from the mRNA gene product. In further embodiments, the gene product is a polypeptide.

In another aspect, the invention provides a method of inhibiting growth of a tumor cell by modulating expression of a gene product, where the gene product is encoded by a gene identified by a sequence selected from the group consisting of: SEQ ID NOS: 1, 3, 5, 7, 9, 11-13, 15, 16, 18, 20, 22, 24, 26, 27, 29 and 128-1618.

These and other objects, advantages, and features of the invention will become apparent to those persons skilled in the art upon reading the details of the invention as more fully described below.

### BRIEF DESCRIPTION OF THE FIGURES

FIG. 1 is a graph showing the message levels of the gene corresponding to SK2 (c9083, SEQ ID NO:3) in the indicated cell lines.

FIG. 2 is a graph showing the effect of SK2 (9083) antisense oligonucleotides upon message levels for the gene corresponding to SK2 (SEQ ID NO:3).

FIG. 3 is a graph showing the effect of SK2 (9083) antisense oligonucleotides upon proliferation of SW620 cells.

FIG. 4 is a graph showing the effect of SK2 (9083) antisense oligonucleotides upon proliferation of a non-colon cell line, HT1080.

FIG. 5 is a graph showing the effect of antisense oligonucleotides to the gene corresponding to cluster 378805 upon growth of SW620 cells (31-4 as: antisense; 31-4rc: reverse control; WT: wild type control (no oligo)).

FIG. 6 is a graph showing the results of proliferation assay with SW620 assays to examine the effects of expression of K-Ras (control).

FIG. 7 is a graph showing the results of proliferation assay with SW620 assays to examine the effects of expression of, the gene corresponding to c3376 (CHIR11-4).

FIG. 8 is a graph showing the results of proliferation assay with SW620 assays to examine the effects of expression of the gene corresponding to 402380 (CHIR33-4).

FIG. 9 is a graph showing the effects of expression of genes corresponding to K-Ras (control) and to 402380 (CHIR33-4) upon colon formation of SW620 cells in soft agar (values normalized to WST1).

### DETAILED DESCRIPTION OF THE INVENTION

The present invention provides polynucleotides, as well as polypeptides encoded thereby, that are differentially expressed in cancer cells. Methods are provided in which these polynucleotides and polypeptides are used for detecting and reducing the growth of cancer cells. Also provided are methods in which the polynucleotides and polypeptides of the invention are used in a variety of diagnostic and therapeutic applications for cancer. The invention finds use in the prevention, treatment, detection or research into any cancer, including prostrate, pancreas, colon, brain, lung, breast, bone, skin cancers. For example, the invention finds use in the prevention, treatment, detection of or research into endocrine system cancers, such as cancers of the thyroid, pituitary, and adrenal glands and the pancreatic islets; gastrointestinal cancers, such as cancer of the anus, colon, esophagus, gallbladder, stomach, liver, and rectum; genitourinary cancers such as cancer of the penis, prostate and testes; gynecological cancers, such as cancer of the ovaries, cervix, endometrium, uterus, fallopian tubes, vagina, and vulva; head and neck cancers, such as hypopharyngeal, laryngeal, oropharyngeal cancers, lip, mouth and oral cancers, cancer of the salivary gland, cancer of the digestive tract and sinus cancer; leukemia; lymphomas including Hodgkin's and non-Hodgkin's lymphoma; metastatic cancer; myelomas; sarcomas; skin cancer; urinary tract cancers including bladder, kidney and urethral cancers; and pediatric cancers, such as pediatric brain tumors, leukemia, lymphomas, sarcomas, liver cancer and neuroblastoma and retinoblastoma.

Before the present invention is described, it is to be understood that this invention is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications and patent applications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a polynucleotide” includes a plurality of such polynucleotides and reference to “the cancer cell” includes reference to one or more cells and equivalents thereof known to those skilled in the art, and so forth.

The publications and applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “polynucleotide” and “nucleic acid”, used interchangeably herein, refer to polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These terms further include, but are not limited to, mRNA or cDNA that comprise intronic sequences (see, e.g., Niwa et al. (1999) Cell 99(7):691-702). The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. The term “polynucleotide” also encompasses peptidic nucleic acids (Pooga et al Curr Cancer Drug Targets. (2001) 1:231-9).

A “gene product” is a biopolymeric product that is expressed or produced by a gene. A gene product may be, for example, an unspliced RNA, an mRNA, a splice variant mRNA, a polypeptide, a post-translationally modified polypeptide, a splice variant polypeptide etc. Also encompassed by this term is biopolymeric products that are made using an RNA gene product as a template (i.e. cDNA of the RNA). A gene product may be made enzymatically, recombinantly, chemically, or within a cell to which the gene is native. In many embodiments, if the gene product is proteinaceous, it exhibits a biological activity. In many embodiments, if the gene product is a nucleic acid, it can be translated into a proteinaceous gene product that exhibits a biological activity.

A composition (e.g. a polynucleotide, polypeptide, antibody, or host cell) that is “isolated” or “in substantially isolated form” refers to a composition that is in an environment different from that in which the composition naturally occurs. For example, a polynucleotide that is in substantially isolated form is outside of the host cell in which the polynucleotide naturally occurs, and could be a purified fragment of DNA, could be part of a heterologous vector, or could be contained within a host cell that is not a host cell from which the polynucleotide naturally occurs. The term “isolated” does not refer to a genomic or cDNA library, whole cell total protein or mRNA preparation, genomic DNA preparation, or an isolated human chromosome. A composition which is in substantially isolated form is usually substantially purified.

As used herein, the term “substantially purified” refers to a compound (e.g., a polynucleotide, a polypeptide or an antibody, etc.) that is removed from its natural environment and is usually at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight. In the case of polynucleotides, “A” and “B” may be two different genes positioned on different chromosomes or adjacently on the same chromosome, or two isolated cDNA species, for example.

The terms “polypeptide” and “protein”, interchangeably used herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

“Heterologous” refers to materials that are derived from different sources (e.g., from different genes, different species, etc.).

As used herein, the terms “a gene that is differentially expressed in a cancer cell,” and “a polynucleotide that is differentially expressed in a cancer cell” are used interchangeably herein, and generally refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source.

“Differentially expressed polynucleotide” as used herein refers to a nucleic acid molecule (RNA or DNA) comprising a sequence that represents a differentially expressed gene, e.g., the differentially expressed polynucleotide comprises a sequence (e.g., an open reading frame encoding a gene product; a non-coding sequence) that uniquely identifies a differentially expressed gene so that detection of the differentially expressed polynucleotide in a sample is correlated with the presence of a differentially expressed gene in a sample. “Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

“Corresponds to” or “represents” when used in the context of, for example, a polynucleotide or sequence that “corresponds to” or “represents” a gene means that at least a portion of a sequence of the polynucleotide is present in the gene or in the nucleic acid gene product (e.g., mRNA or cDNA). A subject nucleic acid may also be “identified” by a polynucleotide if the polynucleotide corresponds to or represents the gene. Genes identified by a polynucleotide may have all or a portion of the identifying sequence wholly present within an exon of a genomic sequence of the gene, or different portions of the sequence of the polynucleotide may be present in different exons (e.g., such that the contiguous polynucleotide sequence is present in an mRNA, either pre- or post-splicing, that is an expression product of the gene). In some embodiments, the polynucleotide may represent or correspond to a gene that is modified in a cancerous cell relative to a normal cell. The gene in the cancerous cell may contain a deletion, insertion, substitution, or translocation relative to the polynucleotide and may have altered regulatory sequences, or may encode a splice variant gene product, for example. The gene in the cancerous cell may be modified by insertion of an endogenous retrovirus, a transposable element, or other naturally occurring or non-naturally occurring nucleic acid. In most cases, a polynucleotide corresponds to or represents a gene if the sequence of the polynucleotide is most identical to the sequence of a gene or its product (e.g. mRNA or cDNA) as compared to other genes or their products. In most embodiments, the most identical gene is determined using a sequence comparison of a polynucleotide to a database of polynucleotides (e.g. GenBank) using the BLAST program at default settings For example, if the most similar gene in the human genome to an exemplary polynucleotide is the protein kinase C gene, the exemplary polynucleotide corresponds to protein kinase C. In most cases, the sequence of a fragment of an exemplary polynucleotide is at least 95%, 96%, 97%, 98%, 99% or up to 100% identical to a sequence of at least 15, 20, 25, 30, 35, 40, 45, or 50 contiguous nucleotides of a corresponding gene or its product (mRNA or cDNA), when nucleotides that are “N” represent G, A, T or C.

An “identifying sequence” is a minimal fragment of a sequence of contiguous nucleotides that uniquely identifies or defines a polynucleotide sequence or its complement. In many embodiments, a fragment of a polynucleotide uniquely identifies or defines a polynucleotide sequence or its complement. In some embodiments, the entire contiguous sequence of a gene, cDNA, EST, or other provided sequence is an identifying sequence.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

As used herein, the term “a polypeptide associated with cancer” refers to a polypeptide encoded by a polynucleotide that is differentially expressed in a cancer cell.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and the like.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest.

The term “normal” as used in the context of “normal cell,” is meant to refer to a cell of an untransformed phenotype or exhibiting a morphology of a non-transformed cell of the tissue type being examined.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” generally refers to a gene or gene product that, upon modulation of its activity (e.g., by modulation of expression, biological activity, and the like), can provide for modulation of the cancerous phenotype.

As used throughout, “modulation” is meant to refer to an increase or a decrease in the indicated phenomenon (e.g., modulation of a biological activity refers to an increase in a biological activity or a decrease in a biological activity).

Polynucleotide Compositions

The present invention provides isolated polynucleotides that contain nucleic acids that are differentially expressed in cancer cells. The polynucleotides, as well as any polypeptides encoded thereby, find use in a variety of therapeutic and diagnostic methods.

The scope of the invention with respect to compositions containing the isolated polynucleotides useful in the methods described herein includes, but is not necessarily limited to, polynucleotides having (i.e., comprising) a sequence set forth in any one of the polynucleotide sequences provided herein, or fragment thereof; polynucleotides obtained from the biological materials described herein or other biological sources (particularly human sources) by hybridization under stringent conditions (particularly conditions of high stringency); genes corresponding to the provided polynucleotides; cDNAs corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicated.

The invention features polynucleotides that represent genes that are expressed in human tissue, specifically polynucleotides that are differentially expressed in tissues containing cancerous cells. Nucleic acid compositions described herein of particular interest are at least about 15 bp in length, at least about 30 bp in length, at least about 50 bp in length, at least about 100 bp, at least about 200 bp in length, at least about 300 bp in length, at least about 500 bp in length, at least about 800 bp in length, at least about 1 kb in length, at least about 2.0 kb in length, at least about 3.0 kb in length, at least about 5 kb in length, at least about 10 kb in length, at least about 50 kb in length and are usually less than about 200 kb in length. These polynucleotides (or polynucleotide fragments) have uses that include, but are not limited to, diagnostic probes and primers as starting materials for probes and primers, as discussed herein.

The subject polynucleotides usually comprise a sequence set forth in any one of the polynucleotide sequences provided herein, for example, in the sequence listing, incorporated by reference in a table (e.g. by an NCBI accession number), a cDNA deposited at the A.T.C.C., or a fragment or variant thereof. A “fragment” or “portion” of a polynucleotide is a contiguous sequence of residues at least about 10 nt to about 12 nt, 15 nt, 16 nt, 18 nt or 20 nt in length, usually at least about 22 nt, 24 nt, 25 nt, 30 nt, 40 nt, 50 nt, 60 nt, 70 nt, 80 nt, 90 nt, 100 nt to at least about 150 nt, 200 nt, 250 nt, 300 nt, 350 nt, 400 nt, 500 nt, 800 nt or up to about 1000 nt, 1500 or 2000 nt in length. In some embodiments, a fragment of a polynucleotide is the coding sequence of a polynucleotide. A fragment of a polynucleotide may start at position 1 (i.e. the first nucleotide) of a nucleotide sequence provided herein, or may start at about position 10, 20, 30, 50, 75, 100, 150, 200, 250, 300, 350, 400, 450, 500, 600, 700, 800, 900, 1000, 1500 or 2000, or an ATG translational initiation codon of a nucleotide sequence provided herein. In this context “about” includes the particularly recited value or a value larger or smaller by several (5, 4, 3, 2, or 1) nucleotides. The described polynucleotides and fragments thereof find use as hybridization probes, PCR primers, BLAST probes, or as an identifying sequence, for example.

The subject nucleic acids may be variants or degenerate variants of a sequence provided herein. In general, a variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence. as determined by the Smith-Waterman homology search algorithm as implemented in MPSRCH program (Oxford Molecular). For the purposes of this invention, a preferred method of calculating percent identity is the Smith-Waterman algorithm. Global DNA sequence identity should be greater than 65% as determined by the Smith-Waterman homology search algorithm as implemented in MPSRCH program (Oxford Molecular) using an gap search with the following search parameters: gap open penalty, 12; and gap extension penalty, 1.

The subject nucleic acid compositions include full-length cDNAs or mRNAs that encompass an identifying sequence of contiguous nucleotides from any one of the polynucleotide sequences provided herein.

As discussed above, the polynucleotides useful in the methods described herein also include polynucleotide variants having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

In one embodiment, hybridization is performed using a fragment of at least 15 contiguous nucleotides (nt) of at least one of the polynucleotide sequences provided herein. That is, when at least 15 contiguous nt of one of the disclosed polynucleotide sequences is used as a probe, the probe will preferentially hybridize with a nucleic acid comprising the complementary sequence, allowing the identification and retrieval of the nucleic acids that uniquely hybridize to the selected probe. Probes from more than one polynucleotide sequence provided herein can hybridize with the same nucleic acid if the cDNA from which they were derived corresponds to one mRNA.

Polynucleotides contemplated for use in the invention also include those having a sequence of naturally occurring variants of the nucleotide sequences (e.g., degenerate variants (e.g., sequences that encode the same polypeptides but, due to the degenerate nature of the genetic code, different in nucleotide sequence), allelic variants, etc.). Variants of the polynucleotides contemplated by the invention are identified by hybridization of putative variants with nucleotide sequences disclosed herein, preferably by hybridization under stringent conditions. For example, by using appropriate wash conditions, variants of the polynucleotides described herein can be identified where the allelic variant exhibits at most about 25-30% base pair (bp) mismatches relative to the selected polynucleotide probe. In general, allelic variants contain 15-25% bp mismatches, and can contain as little as even 5-15%, or 2-5%, or 1-2% bp mismatches, as well as a single bp mismatch.

The invention also encompasses homologs corresponding to any one of the polynucleotide sequences provided herein, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 80%%, at least 85, at least 90%, at least 95%, at least 96%, at least 97%, at least 98%, at least 99% or even 100% identity between nucleotide sequences. Sequence similarity is calculated based on a reference sequence, which may be a subset of a larger sequence, such as a conserved motif, coding region, flanking region, etc. A reference sequence will usually be at least about a fragment of a polynucleotide sequence and may extend to the complete sequence that is being compared. Algorithms for sequence analysis are known in the art, such as gapped BLAST, described in Altschul, et al. Nucleic Acids Res. (1997) 25:3389-3402, or TeraBLAST available from TimeLogic Corp. (Crystal Bay, Nev.).

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acid compositions of the subject invention can encode all or a part of the naturally-occurring polypeptides. Double or single stranded fragments can be obtained from the DNA sequence by chemically synthesizing oligonucleotides in accordance with conventional methods, by restriction enzyme digestion, by PCR amplification, etc.

Probes specific to the polynucleotides described herein can be generated using the polynucleotide sequences disclosed herein. The probes are usually a fragment of a polynucleotide sequences provided herein. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein. More preferably, probes are designed based on a contiguous sequence of one of the subject polynucleotides that remain unmasked following application of a masking program for masking low complexity (e.g., XBLAST, RepeatMasker, etc.) to the sequence, i.e., one would select an unmasked region, as indicated by the polynucleotides outside the poly-n stretches of the masked sequence produced by the masking program.

The polynucleotides of interest in the subject invention are isolated and obtained in substantial purity, generally as other than an intact chromosome. Usually, the polynucleotides, either as DNA or RNA, will be obtained substantially free of other naturally-occurring nucleic acid sequences that they are usually associated with, generally being at least about 50%, usually at least about 90% pure and are typically “recombinant”, e.g., flanked by one or more nucleotides with which it is not normally associated on a naturally occurring chromosome.

The polynucleotides described herein can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The nucleic acid compositions described herein can be used to, for example, produce polypeptides, as probes for the detection of mRNA in biological samples (e.g., extracts of human cells) or cDNA produced from such samples, to generate additional copies of the polynucleotides, to generate ribozymes or antisense oligonucleotides, and as single stranded DNA probes or as triple-strand forming oligonucleotides. The probes described herein can be used to, for example, determine the presence or absence of any one of the polynucleotide provided herein or variants thereof in a sample. These and other uses are described in more detail below.

Polypeptides and Variants Thereof

The present invention further provides polypeptides encoded by polynucleotides that represent genes that are differentially expressed in cancer cells. Such polypeptides are referred to herein as “polypeptides associated with cancer.” The polypeptides can be used to generate antibodies specific for a polypeptide associated with cancer, which antibodies are in turn useful in diagnostic methods, prognostics methods, therametric methods, and the like as discussed in more detail herein. Polypeptides are also useful as targets for therapeutic intervention, as discussed in more detail herein.

The polypeptides contemplated by the invention include those encoded by the disclosed polynucleotides and the genes to which these polynucleotides correspond, as well as nucleic acids that, by virtue of the degeneracy of the genetic code, are not identical in sequence to the disclosed polynucleotides. Further polypeptides contemplated by the invention include polypeptides that are encoded by polynucleotides that hybridize to polynucleotide of the sequence listing. Thus, the invention includes within its scope a polypeptide encoded by a polynucleotide having the sequence of any one of the polynucleotide sequences provided herein, or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide described herein, as measured by BLAST 2.0 using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein as identified above, where sequence identity is determined using the BLAST 2.0 algorithm, with the parameters described supra.

In general, the polypeptides of interest in the subject invention are provided in a non-naturally occurring environment, e.g. are separated from their naturally occurring environment. In certain embodiments, the subject protein is present in a composition that is enriched for the protein as compared to a cell or extract of a cell that naturally produces the protein. As such, isolated polypeptide is provided, where by “isolated” or “in substantially isolated form” is meant that the protein is present in a composition that is substantially free of other polypeptides, where by substantially free is meant that less than 90%, usually less than 60% and more usually less than 50% of the composition is made up of other polypeptides of a cell that the protein is naturally found.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted.

Variants can be designed so as to retain or have enhanced biological activity of a particular region of the protein (e.g., a functional domain and/or, where the polypeptide is a member of a protein family, a region associated with a consensus sequence). For example, muteins can be made which are optimized for increased antigenicity, i.e. amino acid variants of a polypeptide may be made that increase the antigenicity of the polypeptide. Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579). Cysteine-depleted muteins can be produced as disclosed in U.S. Pat. No. 4,959,314. Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants.

A fragment of a subject polypeptide is, for example, a polypeptide having an amino acid sequence which is a portion of a subject polypeptide e.g. a polypeptide encoded by a subject polynucleotide that is identified by any one of the sequence of SEQ ID NOS 1, 3, 5, 7, 9, 11-13, 15, 16, 18, 20, 22, 24, 26, 27, 29 and 128-1618 or its complement. The polypeptide fragments of the invention are preferably at least about 9 aa, at least about 15 aa, and more preferably at least about 20 aa, still more preferably at least about 30 aa, and even more preferably, at least about 40 aa, at least about 50 aa, at least about 75 aa, at least about 100 aa, at least about 125 aa or at least about 150 aa in length. A fragment “at least 20 aa in length,” for example, is intended to include 20 or more contiguous amino acids from, for example, the polypeptide encoded by a cDNA, in a cDNA clone contained in a deposited library, or a nucleotide sequence shown in SEQ ID NOS:1, 3, 5, 7, 9, 11-13, 15, 16, 18, 20, 22, 24, 26, 27, 29 and 128-1618 or the complementary stand thereof. In this context “about” includes the particularly recited value or a value larger or smaller by several (5, 4, 3, 2, or 1) amino acids. These polypeptide fragments have uses that include, but are not limited to, production of antibodies as discussed herein. Of course, larger fragments (e.g., at least 150, 175, 200, 250, 500, 600, 1000, or 2000 amino acids in length) are also encompassed by the invention.

Moreover, representative examples of polypeptides fragments of the invention (useful in, for example, as antigens for antibody production), include, for example, fragments comprising, or alternatively consisting of, a sequence from about amino acid number 1-10, 5-10, 10-20, 21-31, 31-40, 41-61, 61-81, 91-120, 121-140, 141-162, 162-200, 201-240, 241-280, 281-320, 321-360, 360-400, 400-450, 451-500, 500-600, 600-700, 700-800, 800-900 and the like. In this context “about” includes the particularly recited range or a range larger or smaller by several (5, 4, 3, 2, or 1) amino acids, at either terminus or at both termini. In some embodiments, these fragments has a functional activity (e.g., biological activity) whereas in other embodiments, these fragments may be used to make an antibody.

In one example, a polynucleotide having a sequence set forth in the sequence listing, containing no flanking sequences (i.e., consisting of the sequence set forth in the sequence listing), may be cloned into an expression vector having ATG and a stop codon (e.g. any one of the pET vector from Invitrogen, or other similar vectors from other manufactures), and used to express a polypeptide of interest encoded by the polynucleotide in a suitable cell, e.g., a bacterial cell. Accordingly, the polynucleotides may be used to produce polypeptides, and these polypeptides may be used to produce antibodies by known methods described above and below. In many embodiments, the sequence of the encoded polypeptide does not have to be known prior to its expression in a cell. However, if it desirable to know the sequence of the polypeptide, this may be derived from the sequence of the polynucleotide. Using the genetic code, the polynucleotide may be translated by hand, or by computer means. Suitable software for identifying open reading frames and translating them into polypeptide sequences are well know in the art, and include: Lasergene™ from DNAStar (Madison, Wis.), and Vector NTI™ from Informax (Frederick Md.), and the like.

The amino acid sequences of xemplary polypeptides of the invention are shown in SEQ ID NOS: 2, 4, 6, 8, 10, 14, 17, 19, 21, 23, 25, 28 and 1619-1675.

Further polypeptide variants may are described in PCT publications WO/00-55173, WO/01-07611 and WO/02-16429

Vectors, Host Cells and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides of the invention may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria.

Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNHSA, pNH16a, pNH18A, pNH46A, available from Stratagene Cloning Systems, Inc.; and ptrc99a, pKK223-3, pKK233-3, pDR540, pRITS available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carload, Calif.). Other suitable vectors will be readily apparent to the skilled artisan.

Nucleic acids of interest may be cloned into a suitable vector by route methods. Suitable vectors include plasmids, cosmids, recombinant viral vectors e.g. retroviral vectors, YACs, BACs and the like, phage vectors.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Suitable methods and compositions for polypeptide expression may be found in PCT publications WO/00-55173, WO/01-07611 and WO/02-16429, and suitable methods and compositions for production of modified polypeptides may be found in PCT publications WO/00-55173, WO/01-07611 and WO/02-16429.

Antibodies and Other Polypeptide or Polynucleotide Binding Molecules

The present invention further provides antibodies, which may be isolated antibodies, that are specific for a polypeptide encoded by a polynucleotide described herein and/or a polypeptide of a gene that corresponds to a polynucleotide described herein. Antibodies can be provided in a composition comprising the antibody and a buffer and/or a pharmaceutically acceptable excipient. Antibodies specific for a polypeptide associated with cancer are useful in a variety of diagnostic and therapeutic methods, as discussed in detail herein.

Gene products, including polypeptides, mRNA (particularly mRNAs having distinct secondary and/or tertiary structures), cDNA, or complete gene, can be prepared and used for raising antibodies for experimental, diagnostic, and therapeutic purposes. Antibodies may be used to identify a gene corresponding to a polynucleotide. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Antibodies

Further polypeptides of the invention relate to antibodies and T-cell antigen receptors (TCR) which immunospecifically bind a subject polypeptide, subject polypeptide fragment, or variant thereof, and/or an epitope thereof (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably the antibodies are human antigen-binding antibody fragments of the present invention and include, but are not limited to, Fab. Fab′ and F(ab′)2, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from, human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, or by size in contiguous amino acid residues. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹M, 5×10⁻¹⁰ M, 10-10 M, etc.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Methods for making screening, assaying, humanizing, and modifying different types of antibody are well known in the art and may be found in PCT publications WO/00-55173, WO/01-07611 and WO/02-16429.

In addition, the invention further provides polynucleotides comprising a nucleotide sequence encoding an antibody of the invention and fragments thereof. The invention also encompasses polynucleotides that hybridize under stringent or alternatively, under lower stringency hybridization conditions, e.g., as defined supra, to polynucleotides that encode an antibody, preferably, that specifically binds to a polypeptide of the invention, preferably, an antibody that binds to a subject polypeptide.

The antibodies of the invention can be produced by any method known in the art for the synthesis of antibodies, in particular, by chemical synthesis or preferably, by recombinant expression techniques. Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

Antibodies production is well known in the art. Exemplary methods and compositions for making antibodies may be found in PCT publications WO/00-55173, WO/01-07611 and WO/02-16429.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)).

These techniques allow for the screening of particular populations of cells, such as might be found with hematological malignancies (i.e. minimal residual disease (MRD) in acute leukemic patients) and “non-self cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Kits

Also provided by the subject invention are kits for practicing the subject methods, as described above. The subject kits include at least one or more of: a subject nucleic acid, isolated polypeptide or an antibody thereto. Other optional components of the kit include: restriction enzymes, control primers and plasmids; buffers, cells, carriers adjuvents etc. The nucleic acids of the kit may also have restrictions sites, multiple cloning sites, primer sites, etc to facilitate their ligation other plasmids. The various components of the kit may be present in separate containers or certain compatible components may be precombined into a single container, as desired. In many embodiments, kits with unit doses of the active agent, e.g. in oral or injectable doses, are provided. In certain embodiments, controls, such as samples from a cancerous or non-cancerous cell are provided by the invention. Further embodiments of the kit include an antibody for a subject polypeptide and a chemotherapeutic agent to be used in combination with the polypeptide as a treatment.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. For example, in the instant case, the sequences of polynucleotides and polypeptides corresponding to genes differentially expressed in cancer, as well as the nucleic acid and amino acid sequences of the genes themselves, can be provided in electronic form in a computer database.

In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a cancerous cell affected by cancer relative to a normal (i.e., substantially disease-free) cell.

The nucleotide sequence information of the library can be embodied in any suitable form, e.g., electronic or biochemical forms. For example, a library of sequence information embodied in electronic form comprises an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention generally comprise sequence information of a plurality of polynucleotide sequences, where at least one of the polynucleotides has a sequence of any of sequence described herein. By plurality is meant at least 2, usually at least 3 and can include up to all of the sequences described herein. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of the sequences described herein, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media.

One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of libraries comprising one or more sequence described herein can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information (e.g. the NCBI sequence database) is publicly available. For example, the gapped BLAST (Altschul et al., Nucleic Acids Res. (1997) 25:3389-3402) and BLAZE (Brutlag et al., Comp. Chem. (1993) 17:203) search algorithms on a Sybase system, or the TeraBLAST (TimeLogic, Crystal Bay, Nev.) program optionally running on a specialized computer platform available from TimeLogic, can be used to identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif, or expression levels of a polynucleotide in a sample, with the stored sequence information. Search means can be used to identify fragments or regions of the genome that match a particular target sequence or target motif. A variety of known algorithms are publicly known and commercially available, e.g. MacPattern (EMBL), TeraBLAST (TimeLogic), BLASTN and BLASTX (NCBI). A “target sequence” can be any polynucleotide or amino acid sequence of six or more contiguous nucleotides or two or more amino acids, preferably from about 10 to 100 amino acids or from about 30 to 300 nt. A variety of means for comparing nucleic acids or polypeptides may be used to compare accomplish a sequence comparison (e.g., to analyze target sequences, target motifs, or relative expression levels) with the data storage means. A skilled artisan can readily recognize that any one of the publicly available homology search programs can be used to search the computer based systems of the present invention to compare of target sequences and motifs. Computer programs to analyze expression levels in a sample and in controls are also known in the art.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but are not limited to, enzyme active sites and signal sequences, kinase domains, receptor binding domains, SH2 domains, SH3 domains, phosphorylation sites, protein interaction domains, transmembrane domains, etc. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile. A gene expression profile can be generated from, for example, a cDNA library prepared from mRNA isolated from a test cell suspected of being cancerous or pre-cancerous, comparing the sequences or partial sequences of the clones against the sequences in an electronic database, where the sequences of the electronic database represent genes differentially expressed in a cancerous cell, e.g., a cancerous breast cell. The number of clones having a sequence that has substantial similarity to a sequence that represents a gene differentially expressed in a cancerous cell is then determined, and the number of clones corresponding to each of such genes is determined. An increased number of clones that correspond to differentially expressed gene is present in the cDNA library of the test cell (relative to, for example, the number of clones expected in a cDNA of a normal cell) indicates that the test cell is cancerous.

As discussed above, the “library” as used herein also encompasses biochemical libraries of the polynucleotides of the sequences described herein, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of the genes described herein is represented by a sequence on the array. By array is meant an article of manufacture that has at least a substrate with at least two distinct nucleic acid targets on one of its surfaces, where the number of distinct nucleic acids can be considerably higher, typically being at least 10 nt, usually at least 20 nt and often at least 25 nt. A variety of different array formats have been developed and are known to those of skill in the art. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the polypeptides of the library will represent at least a portion of the polypeptides encoded by a gene corresponding to a sequence described herein.

Diagnostic and Other Methods Involving Detection of Differentially Expressed Genes

The present invention provides methods of using the polynucleotides described herein in, for example, diagnosis of cancer and classification of cancer cells according to expression profiles. In specific non-limiting embodiments, the methods are useful for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of a polynucleotide that is differentially expressed in a cancer cell, and/or detection of a polypeptide encoded by a polynucleotide that is differentially expressed in a cancer cell (“a polypeptide associated with cancer”). The detection methods of the invention can be conducted in vitro or in vivo, on isolated cells, or in whole tissues or a bodily fluid, e.g., blood, plasma, serum, urine, and the like).

In general, methods of the invention involving detection of a gene product (e.g., mRNA, cDNA generated from such mRNA, and polypeptides) involve contacting a sample with a probe specific for the gene product of interest. “Probe” as used herein in such methods is meant to refer to a molecule that specifically binds a gene product of interest (e.g., the probe binds to the target gene product with a specificity sufficient to distinguish binding to target over non-specific binding to non-target (background) molecules). “Probes” include, but are not necessarily limited to, nucleic acid probes (e.g., DNA, RNA, modified nucleic acid, and the like), antibodies (e.g., antibodies, antibody fragments that retain binding to a target epitope, single chain antibodies, and the like), or other polypeptide, peptide, or molecule (e.g., receptor ligand) that specifically binds a target gene product of interest.

The probe and sample suspected of having the gene product of interest are contacted under conditions suitable for binding of the probe to the gene product. For example, contacting is generally for a time sufficient to allow binding of the probe to the gene product (e.g., from several minutes to a few hours), and at a temperature and conditions of osmolarity and the like that provide for binding of the probe to the gene product at a level that is sufficiently distinguishable from background binding of the probe (e.g., under conditions that minimize non-specific binding). Suitable conditions for probe-target gene product binding can be readily determined using controls and other techniques available and known to one of ordinary skill in the art.

In this embodiment, the probe can be an antibody or other polypeptide, peptide, or molecule (e.g., receptor ligand) that specifically binds a target polypeptide of interest.

The detection methods can be provided as part of a kit. Thus, the invention further provides kits for detecting the presence and/or a level of a polynucleotide that is differentially expressed in a cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a cancer cell comprise a moiety that specifically binds the polypeptide, which may be a specific antibody. The kits of the invention for detecting a polynucleotide that is differentially expressed in a cancer cell comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information.

Detecting a Polypeptide Encoded by a Polynucleotide that is Differentially Expressed in a Cancer Cell

In some embodiments, methods are provided for a detecting cancer cell by detecting in a cell, a polypeptide encoded by a gene differentially expressed in a cancer cell. Any of a variety of known methods can be used for detection, including, but not limited to, immunoassay, using an antibody specific for the encoded polypeptide, e.g., by enzyme-linked immunosorbent assay (ELISA), radioimmunoassay (RIA), and the like; and functional assays for the encoded polypeptide, e.g., binding activity or enzymatic activity.

For example, an immunofluorescence assay can be easily performed on cells without first isolating the encoded polypeptide. The cells are first fixed onto a solid support, such as a microscope slide or microtiter well. This fixing step can permeabilize the cell membrane. The permeablization of the cell membrane permits the polypeptide-specific probe (e.g, antibody) to bind. Alternatively, where the polypeptide is secreted or membrane-bound, or is otherwise accessible at the cell-surface (e.g., receptors, and other molecule stably-associated with the outer cell membrane or otherwise stably associated with the cell membrane, such permeabilization may not be necessary.

Next, the fixed cells are exposed to an antibody specific for the encoded polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells which express the encoded polypeptide will be fluorescently labeled and easily visualized under the microscope. See, for example, Hashido et al. (1992) Biochem. Biophys. Res. Comm. 187:1241-1248.

As will be readily apparent to the ordinarily skilled artisan upon reading the present specification, the detection methods and other methods described herein can be varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample.

The present invention further provides methods for detecting the presence of and/or measuring a level of a polypeptide in a biological sample, which polypeptide is encoded by a polynucleotide that represents a gene differentially expressed in cancer, particularly in a polynucleotide that represents a gene differentially cancer cell, using a probe specific for the encoded polypeptide. In this embodiment, the probe can be a an antibody or other polypeptide, peptide, or molecule (e.g., receptor ligand) that specifically binds a target polypeptide of interest.

The methods generally comprise: a) contacting the sample with an antibody specific for a differentially expressed polypeptide in a test cell; and b) detecting binding between the antibody and molecules of the sample. The level of antibody binding (either qualitative or quantitative) indicates the cancerous state of the cell. For example, where the differentially expressed gene is increased in cancerous cells, detection of an increased level of antibody binding to the test sample relative to antibody binding level associated with a normal cell indicates that the test cell is cancerous.

Suitable controls include a sample known not to contain the encoded polypeptide; and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay.

In general, the specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like.

The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where cancer cells are present. In these embodiments, a detectably-labeled moiety, e.g., an antibody, which is specific for a cancer-associated polypeptide is administered to an individual (e.g., by injection), and labeled cells are located using standard imaging techniques, including, but not limited to, magnetic resonance imaging, computed tomography scanning, and the like. In this manner, cancer cells are differentially labeled.

Detecting a Polynucleotide that Represents a Gene Differentially Expressed in a Cancer Cell

In some embodiments, methods are provided for detecting a cancer cell by detecting expression in the cell of a transcript or that is differentially expressed in a cancer cell. Any of a variety of known methods can be used for detection, including, but not limited to, detection of a transcript by hybridization with a polynucleotide that hybridizes to a polynucleotide that is differentially expressed in a cancer cell; detection of a transcript by a polymerase chain reaction using specific oligonucleotide primers; in situ hybridization of a cell using as a probe a polynucleotide that hybridizes to a gene that is differentially expressed in a cancer cell and the like.

In many embodiments, the levels of a subject gene product are measured. By measured is meant qualitatively or quantitatively estimating the level of the gene product in a first biological sample either directly (e.g. by determining or estimating absolute levels of gene product) or relatively by comparing the levels to a second control biological sample. In many embodiments the second control biological sample is obtained from an individual not having not having cancer. As will be appreciated in the art, once a standard control level of gene expression is known, it can be used repeatedly as a standard for comparison. Other control samples include samples of cancerous tissue.

The methods can be used to detect and/or measure mRNA levels of a gene that is differentially expressed in a cancer cell. In some embodiments, the methods comprise: a) contacting a sample with a polynucleotide that corresponds to a differentially expressed gene described herein under conditions that allow hybridization; and b) detecting hybridization, if any. Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell. Conditions that allow hybridization are known in the art, and have been described in more detail above.

Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Polynucleotides described herein are used for a variety of purposes, such as probes for detection of and/or measurement of, transcription levels of a polynucleotide that is differentially expressed in a cancer cell. Additional disclosure about preferred regions of the disclosed polynucleotide sequences is found in the Examples. A probe that hybridizes specifically to a polynucleotide disclosed herein should provide a detection signal at least 2-, 5-, 10-, or 20-fold higher than the background hybridization provided with other unrelated sequences. It should be noted that “probe” as used in this context of detection of nucleic acid is meant to refer to a polynucleotide sequence used to detect a differentially expressed gene product in a test sample. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Labeled nucleic acid probes may be used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Other examples of nucleotide hybridization assays are described in WO92/02526 and U.S. Pat. No. 5,124,246.

PCR is another means for detecting small amounts of target nucleic acids, methods for which may be found in Sambrook, et al. Molecular Cloning: A Laboratory Manual, CSH Press 1989, pp. 14.2-14.33.

A detectable label may be included in the amplification reaction. Suitable detectable labels include fluorochromes, (e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Arrays

Polynucleotide arrays provide a high throughput technique that can assay a large number of polynucleotides or polypeptides in a sample. This technology can be used as a tool to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734. In most embodiments, the “probe” is detectably labeled. In other embodiments, the probe is immobilized on the array and not detectably labeled.

Arrays can be used, for example, to examine differential expression of genes and can be used to determine gene function. For example, arrays can be used to detect differential expression of a gene corresponding to a polynucleotide described herein, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay, Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Diagnosis, Prognosis, Assessment of Therapy (Therametrics), and Management of Cancer

The polynucleotides described herein, as well as their gene products and corresponding genes and gene products, are of particular interest as genetic or biochemical markers (e.g., in blood or tissues) that will detect the earliest changes along the carcinogenesis pathway and/or to monitor the efficacy of various therapies and preventive interventions.

For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor. These therapies include antibody targeting, antagonists (e.g., small molecules), and gene therapy.

Determining expression of certain polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides described herein, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for cancer can also have clinical implications for metastatic breast cancer, colon cancer, or ovarian cancer, etc.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides and corresponding genes and gene products described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

One type of breast cancer is ductal carcinoma in situ (DCIS): DCIS is when the breast cancer cells are completely contained within the breast ducts (the channels in the breast that carry milk to the nipple), and have not spread into the surrounding breast tissue. This may also be referred to as non-invasive or intraductal cancer, as the cancer cells have not yet spread into the surrounding breast tissue and so usually have not spread into any other part of the body.

Lobular carcinoma in situ breast cancer (LCIS) means that cell changes are found in the lining of the lobules of the breast. It can be present in both breasts. It is also referred to as non-invasive cancer as it has not spread into the surrounding breast tissue.

Invasive breast cancer can be staged as follows: Stage 1 tumours: these measure less than two centimetres. The lymph glands in the armpit are not affected and there are no signs that the cancer has spread elsewhere in the body; Stage 2 tumours: these measure between two and five centimetres, or the lymph glands in the armpit are affected, or both. However, there are no signs that the cancer has spread further; Stage 3 tumours: these are larger than five centimetres and may be attached to surrounding structures such as the muscle or skin. The lymph glands are usually affected, but there are no signs that the cancer has spread beyond the breast or the lymph glands in the armpit; Stage 4 tumours: these are of any size, but the lymph glands are usually affected and the cancer has spread to other parts of the body. This is secondary breast cancer.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well-differentiated or low-grade tumors.

The polynucleotides of the Sequence Listing, and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Low grade means that the cancer cells look very like the normal cells. They are usually slowly growing and are less likely to spread. In high grade tumors the cells look very abnormal. They are likely to grow more quickly and are more likely to spread.

Assessment of proliferation of cells in tumor. The differential expression level of the polynucleotides described herein can facilitate assessment of the rate of proliferation of tumor cells, and thus provide an indicator of the aggressiveness of the rate of tumor growth. For example, assessment of the relative expression levels of genes involved in cell cycle can provide an indication of cellular proliferation, and thus serve as a marker of proliferation.

Detection of Cancer.

The polynucleotides corresponding to genes that exhibit the appropriate expression pattern can be used to detect cancer in a subject. The expression of appropriate polynucleotides can be used in the diagnosis, prognosis and management of cancer. Detection of cancer can be determined using expression levels of any of these sequences alone or in combination with the levels of expression of other known cancer genes. Determination of the aggressive nature and/or the metastatic potential of a cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon E R, et al., Cell (1990) 61(5):759; Hamilton S R et al., Cancer (1993) 72:957; Bodmer W, et al., Nat Genet. (1994) 4(3):217; Fearon E R, Ann N Y Acad Sci. (1995) 768:101). For example, development of cancer can be detected by examining the level of expression of a gene corresponding to a polynucleotides described herein to the levels of oncogenes (e.g. ras) or tumor suppressor genes (e.g. FAP or p53). Thus expression of specific marker polynucleotides can be used to discriminate between normal and cancerous tissue, to discriminate between cancers with different cells of origin, to discriminate between cancers with different potential metastatic rates, etc. For a review of other markers of cancer, see, e.g., Hanahan et al. (2000) Cell 100:57-70.

Treatment of Cancer

The invention further provides methods for reducing growth of cancer cells. The methods provide for decreasing the expression of a gene that is differentially expressed in a cancer cell or decreasing the level of and/or decreasing an activity of a cancer-associated polypeptide. In general, the methods comprise contacting a cancer cell with a substance that modulates (1) expression of a gene that is differentially expressed in cancer; or (2) a level of and/or an activity of a cancer-associated polypeptide.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with breast cancer (e.g., PSA).

The present invention provides methods for treating cancer, generally comprising administering to an individual in need thereof a substance that reduces cancer cell growth, in an amount sufficient to reduce cancer cell growth and treat the cancer. Whether a substance, or a specific amount of the substance, is effective in treating cancer can be assessed using any of a variety of known diagnostic assays for cancer, including, but not limited to, proctoscopy, rectal examination, biopsy, contrast radiographic studies, CAT scan, and detection of a tumor marker associated with cancer in the blood of the individual (e.g., PSA (breast-specific antigen)). The substance can be administered systemically or locally. Thus, in some embodiments, the substance is administered locally, and cancer growth is decreased at the site of administration. Local administration may be useful in treating, e.g., a solid tumor.

A substance that reduces cancer cell growth can be targeted to a cancer cell. Thus, in some embodiments, the invention provides a method of delivering a drug to a cancer cell, comprising administering a drug-antibody complex to a subject, wherein the antibody is specific for a cancer-associated polypeptide, and the drug is one that reduces cancer cell growth, a variety of which are known in the art. Targeting can be accomplished by coupling (e.g., linking, directly or via a linker molecule, either covalently or non-covalently, so as to form a drug-antibody complex) a drug to an antibody specific for a cancer-associated polypeptide. Methods of coupling a drug to an antibody are well known in the art and need not be elaborated upon herein.

Tumor Classification and Patient Stratification

The invention further provides for methods of classifying tumors, and thus grouping or “stratifying” patients, according to the expression profile of selected differentially expressed genes in a tumor. Differentially expressed genes can be analyzed for correlation with other differentially expressed genes in a single tumor type or across tumor types. Genes that demonstrate consistent correlation in expression profile in a given cancer cell type (e.g., in a cancer cell or type of cancer) can be grouped together, e.g., when one gene is overexpressed in a tumor, a second gene is also usually overexpressed. Tumors can then be classified according to the expression profile of one or more genes selected from one or more groups.

The tumor of each patient in a pool of potential patients can be classified as described above. Patients having similarly classified tumors can then be selected for participation in an investigative or clinical trial of a cancer therapeutic where a homogeneous population is desired. The tumor classification of a patient can also be used in assessing the efficacy of a cancer therapeutic in a heterogeneous patient population. In addition, therapy for a patient having a tumor of a given expression profile can then be selected accordingly.

In another embodiment, differentially expressed gene products (e.g., polypeptides or polynucleotides encoding such polypeptides) may be effectively used in treatment through vaccination. The growth of cancer cells is naturally limited in part due to immune surveillance. Stimulation of the immune system using a particular tumor-specific antigen enhances the effect towards the tumor expressing the antigen. An active vaccine comprising a polypeptide encoded by the cDNA of this invention would be appropriately administered to subjects having an alteration, e.g., overabundance, of the corresponding RNA, or those predisposed for developing cancer cells with an alteration of the same RNA. Polypeptide antigens are typically combined with an adjuvant as part of a vaccine composition. The vaccine is preferably administered first as a priming dose, and then again as a boosting dose, usually at least four weeks later. Further boosting doses may be given to enhance the effect. The dose and its timing are usually determined by the person responsible for the treatment.

The invention also encompasses the selection of a therapeutic regimen based upon the expression profile of differentially expressed genes in the patient's tumor. For example, a tumor can be analyzed for its expression profile of the genes corresponding to SEQ ID NOS:1, 3, 5, 7, 9, 11-13, 15, 16, 18, 20, 22, 24, 26, 27, 29 and 128-1618 as described herein, e.g., the tumor is analyzed to determine which genes are expressed at elevated levels or at decreased levels relative to normal cells of the same tissue type. The expression patterns of the tumor are then compared to the expression patterns of tumors that respond to a selected therapy. Where the expression profiles of the test tumor cell and the expression profile of a tumor cell of known drug responsivity at least substantially match (e.g., selected sets of genes at elevated levels in the tumor of known drug responsivity and are also at elevated levels in the test tumor cell), then the therapeutic agent selected for therapy is the drug to which tumors with that expression pattern respond.

Pattern Matching in Diagnosis Using Arrays

In another embodiment, the diagnostic and/or prognostic methods of the invention involve detection of expression of a selected set of genes in a test sample to produce a test expression pattern (TEP). The TEP is compared to a reference expression pattern (REP), which is generated by detection of expression of the selected set of genes in a reference sample (e.g., a positive or negative control sample). The selected set of genes includes at least one of the genes of the invention, which genes correspond to the polynucleotide sequences described herein. Of particular interest is a selected set of genes that includes gene differentially expressed in the disease for which the test sample is to be screened.

Identification of Therapeutic Targets and Anti-Cancer Therapeutic Agents

The present invention also encompasses methods for identification of agents having the ability to modulate activity of a differentially expressed gene product, as well as methods for identifying a differentially expressed gene product as a therapeutic target for treatment of cancer.

Identification of compounds that modulate activity of a differentially expressed gene product can be accomplished using any of a variety of drug screening techniques. Such agents are candidates for development of cancer therapies. Of particular interest are screening assays for agents that have tolerable toxicity for normal, non-cancerous human cells. The screening assays of the invention are generally based upon the ability of the agent to modulate an activity of a differentially expressed gene product and/or to inhibit or suppress phenomenon associated with cancer (e.g., cell proliferation, colony formation, cell cycle arrest, metastasis, and the like).

Screening of Candidate Agents

Screening assays can be based upon any of a variety of techniques readily available and known to one of ordinary skill in the art. In general, the screening assays involve contacting a cancerous cell with a candidate agent, and assessing the effect upon biological activity of a differentially expressed gene product. The effect upon a biological activity can be detected by, for example, detection of expression of a gene product of a differentially expressed gene (e.g., a decrease in mRNA or polypeptide levels, would in turn cause a decrease in biological activity of the gene product). Alternatively or in addition, the effect of the candidate agent can be assessed by examining the effect of the candidate agent in a functional assay. For example, where the differentially expressed gene product is an enzyme, then the effect upon biological activity can be assessed by detecting a level of enzymatic activity associated with the differentially expressed gene product. The functional assay will be selected according to the differentially expressed gene product. In general, where the differentially expressed gene is increased in expression in a cancerous cell, agents of interest are those that decrease activity of the differentially expressed gene product.

Assays described infra can be readily adapted in the screening assay embodiments of the invention. Exemplary assays useful in screening candidate agents include, but are not limited to, hybridization-based assays (e.g., use of nucleic acid probes or primers to assess expression levels), antibody-based assays (e.g., to assess levels of polypeptide gene products), binding assays (e.g., to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

Identification of Therapeutic Targets

In another embodiment, the invention contemplates identification of differentially expressed genes and gene products as therapeutic targets. In some respects, this is the converse of the assays described above for identification of agents having activity in modulating (e.g., decreasing or increasing) activity of a differentially expressed gene product.

In this embodiment, therapeutic targets are identified by examining the effect(s) of an agent that can be demonstrated or has been demonstrated to modulate a cancerous phenotype (e.g., inhibit or suppress or prevent development of a cancerous phenotype). Such agents are generally referred to herein as an “anti-cancer agent”, which agents encompass chemotherapeutic agents. For example, the agent can be an antisense oligonucleotide that is specific for a selected gene transcript. For example, the antisense oligonucleotide may have a sequence corresponding to a sequence of a differentially expressed gene described herein, e.g., a sequence of one of SEQ ID NOS:1, 3, 5, 7, 9, 11-13, 15, 16, 18, 20, 22, 24, 26, 27, 29 and 128-1618.

Assays for identification of therapeutic targets can be conducted in a variety of ways using methods that are well known to one of ordinary skill in the art. For example, a test cancerous cell that expresses or overexpresses a differentially expressed gene is contacted with an anti-cancer agent, the effect upon a cancerous phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product. The cancerous phenotype can be, for example, cellular proliferation, loss of contact inhibition of growth (e.g., colony formation), tumor growth (in vitro or in vivo), and the like. Alternatively or in addition, the effect of modulation of a biological activity of the candidate target gene upon cell death/apoptosis or cell cycle regulation can be assessed.

Inhibition or suppression of a cancerous phenotype, or an increase in cell death or apoptosis as a result of modulation of biological activity of a candidate gene product indicates that the candidate gene product is a suitable target for cancer therapy. Assays described infra can be readily adapted for assays for identification of therapeutic targets. Generally such assays are conducted in vitro, but many assays can be adapted for in vivo analyses, e.g., in an appropriate, art-accepted animal model of the cancer.

Candidate Agents

The term “agent” as used herein describes any molecule, e.g. protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense and RNAi polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

For method that involve RNAi (RNA interference), a double stranded RNA (dsRNA) molecule is usually used. The dsRNA is prepared to be substantially identical to at least a segment of a subject polynucleotide (e.g. a cDNA or gene). In general, the dsRNA is selected to have at least 70%, 75%, 80%, 85% or 90% sequence identity with the subject polynucleotide over at least a segment of the candidate gene. In other instances, the sequence identity is even higher, such as 95%, 97% or 99%, and in still other instances, there is 100% sequence identity with the subject polynucleotide over at least a segment of the subject polynucleotide. The size of the segment over which there is sequence identity can vary depending upon the size of the subject polynucleotide. In general, however, there is substantial sequence identity over at least 15, 20, 25, 30, 35, 40 or 50 nucleotides. In other instances, there is substantial sequence identity over at least 100, 200, 300, 400, 500 or 1000 nucleotides; in still other instances, there is substantial sequence identity over the entire length of the subject polynucleotide, i.e., the coding and non-coding region of the candidate gene.

Because only substantial sequence similarity between the subject polynucleotide and the dsRNA is necessary, sequence variations between these two species arising from genetic mutations, evolutionary divergence and polymorphisms can be tolerated. Moreover, as described further infra, the dsRNA can include various modified or nucleotide analogs.

Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

The size of the dsRNA that is utilized varies according to the size of the subject polynucleotide whose expression is to be suppressed and is sufficiently long to be effective in reducing expression of the subject polynucleotide in a cell. Generally, the dsRNA is at least 10-15 nucleotides long. In certain applications, the dsRNA is less than 20, 21, 22, 23, 24 or 25 nucleotides in length. In other instances, the dsRNA is at least 50, 100, 150 or 200 nucleotides in length. The dsRNA can be longer still in certain other applications, such as at least 300, 400, 500 or 600 nucleotides. Typically, the dsRNA is not longer than 3000 nucleotides. The optimal size for any particular subject polynucleotide can be determined by one of ordinary skill in the art without undue experimentation by varying the size of the dsRNA in a systematic fashion and determining whether the size selected is effective in interfering with expression of the subject polynucleotide.

dsRNA can be prepared according to any of a number of methods that are known in the art, including in vitro and in vivo methods, as well as by synthetic chemistry approaches.

In vitro methods. Certain methods generally involve inserting the segment corresponding to the candidate gene that is to be transcribed between a promoter or pair of promoters that are oriented to drive transcription of the inserted segment and then utilizing an appropriate RNA polymerase to carry out transcription. One such arrangement involves positioning a DNA fragment corresponding to the candidate gene or segment thereof into a vector such that it is flanked by two opposable polymerase-specific promoters that can be same or different. Transcription from such promoters produces two complementary RNA strands that can subsequently anneal to form the desired dsRNA. Exemplary plasmids for use in such systems include the plasmid (PCR 4.0 TOPO) (available from Invitrogen). Another example is the vector pGEM-T (Promega, Madison, Wis.) in which the oppositely oriented promoters are T7 and SP6; the T3 promoter can also be utilized.

In a second arrangement, DNA fragments corresponding to the segment of the subject polynucleotide that is to be transcribed is inserted both in the sense and antisense orientation downstream of a single promoter. In this system, the sense and antisense fragments are cotranscribed to generate a single RNA strand that is self-complementary and thus can form dsRNA.

Various other in vitro methods have been described. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety.

Single-stranded RNA can also be produced using a combination of enzymatic and organic synthesis or by total organic synthesis. The use of synthetic chemical methods enable one to introduce desired modified nucleotides or nucleotide analogs into the dsRNA.

In vivo methods. dsRNA can also be prepared in vivo according to a number of established methods (see, e.g., Sambrook, et al. (1989) Molecular Cloning: A Laboratory Manual, 2^{nd} ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

Once the single-stranded RNA has been formed, the complementary strands are allowed to anneal to form duplex RNA. Transcripts are typically treated with DNAase and further purified according to established protocols to remove proteins. Usually such purification methods are not conducted with phenol:chloroform. The resulting purified transcripts are subsequently dissolved in RNAase free water or a buffer of suitable composition.

dsRNA is generated by annealing the sense and anti-sense RNA in vitro. Generally, the strands are initially denatured to keep the strands separate and to avoid self-annealing. During the annealing process, typically certain ratios of the sense and antisense strands are combined to facilitate the annealing process. In some instances, a molar ratio of sense to antisense strands of 3:7 is used; in other instances, a ratio of 4:6 is utilized; and in still other instances, the ratio is 1:1.

The buffer composition utilized during the annealing process can in some instances affect the efficacy of the annealing process and subsequent transfection procedure. While some have indicated that the buffered solution used to carry out the annealing process should include a potassium salt such as potassium chloride (e.g. at a concentration of about 80 mM). In some embodiments, the buffer is substantially postassium free. Once single-stranded RNA has annealed to form duplex RNA, typically any single-strand overhangs are removed using an enzyme that specifically cleaves such overhangs (e.g., RNAase A or RNAase T).

Once the dsRNA has been formed, it is introduced into a reference cell, which can include an individual cell or a population of cells (e.g., a tissue, an embryo and an entire organism). The cell can be from essentially any source, including animal, plant, viral, bacterial, fungal and other sources. If a tissue, the tissue can include dividing or nondividing and differentiated or undifferentiated cells. Further, the tissue can include germ line cells and somatic cells. Examples of differentiated cells that can be utilized include, but are not limited to, neurons, glial cells, blood cells, megakaryocytes, lymphocytes, macrophages, neutrophils, eosinophils, basophils, mast cells, leukocytes, granulocytes, keratinocytes, adipocytes, osteoblasts, osteoclasts, hepatocytes, cells of the endocrine or exocrine glands, fibroblasts, myocytes, cardiomyocytes, and endothelial cells. The cell can be an individual cell of an embryo, and can be a blastocyte or an oocyte.

Certain methods are conducted using model systems for particular cellular states (e.g., a disease). For instance, certain methods provided herein are conducted with a cancer cell lines that serves as a model system for investigating genes that are correlated with various cancers.

A number of options can be utilized to deliver the dsRNA into a cell or population of cells such as in a cell culture, tissue or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439).

Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

If the dsRNA is to be introduced into an organism or tissue, gene gun technology is an option that can be employed. This generally involves immobilizing the dsRNA on a gold particle which is subsequently fired into the desired tissue. Research has also shown that mammalian cells have transport mechanisms for taking in dsRNA (see, e.g., Asher, et al. (1969) Nature 223:715-717). Consequently, another delivery option is to administer the dsRNA extracellularly into a body cavity, interstitial space or into the blood system of the mammal for subsequent uptake by such transport processes. The blood and lymph systems and the cerebrospinal fluid are potential sites for injecting dsRNA. Oral, topical, parenteral, rectal and intraperitoneal administration are also possible modes of administration.

The composition introduced can also include various other agents in addition to the dsRNA. Examples of such agents include, but are not limited to, those that stabilize the dsRNA, enhance cellular uptake and/or increase the extent of interference. Typically, the dsRNA is introduced in a buffer that is compatible with the composition of the cell into which the RNA is introduced to prevent the cell from being shocked. The minimum size of the dsRNA that effectively achieves gene silencing can also influence the choice of delivery system and solution composition.

Sufficient dsRNA is introduced into the tissue to cause a detectable change in expression of a taget gene (assuming the candidate gene is in fact being expressed in the cell into which the dsRNA is introduced) using available detection methodologies. Thus, in some instances, sufficient dsRNA is introduced to achieve at least a 5-10% reduction in candidate gene expression as compared to a cell in which the dsRNA is not introduced. In other instances, inhibition is at least 20, 30, 40 or 50%. In still other instances, the inhibition is at least 60, 70, 80, 90 or 95%. Expression in some instances is essentially completely inhibited to undetectable levels.

The amount of dsRNA introduced depends upon various factors such as the mode of administration utilized, the size of the dsRNA, the number of cells into which dsRNA is administered, and the age and size of an animal if dsRNA is introduced into an animal. An appropriate amount can be determined by those of ordinary skill in the art by initially administering dsRNA at several different concentrations for example, for example. In certain instances when dsRNA is introduced into a cell culture, the amount of dsRNA introduced into the cells varies from about 0.5 to 3 μg per 10⁶ cells.

A number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

Use of Polypeptides to Screen for Peptide Analogs and Antagonists

Polypeptides encoded by differentially expressed genes identified herein can be used to screen peptide libraries to identify binding partners, such as receptors, from among the encoded polypeptides. Peptide libraries can be synthesized according to methods known in the art (see, e.g., U.S. Pat. No. 5,010,175 and WO 91/17823).

Agonists or antagonists of the polypeptides of the invention can be screened using any available method known in the art, such as signal transduction, antibody binding, receptor binding, mitogenic assays, chemotaxis assays, etc. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

Such screening and experimentation can lead to identification of a polypeptide binding partner, such as a receptor, encoded by a gene or a cDNA corresponding to a polynucleotide described herein, and at least one peptide agonist or antagonist of the binding partner. Such agonists and antagonists can be used to modulate, enhance, or inhibit receptor function in cells to which the receptor is native, or in cells that possess the receptor as a result of genetic engineering. Further, if the receptor shares biologically important characteristics with a known receptor, information about agonist/antagonist binding can facilitate development of improved agonists/antagonists of the known receptor.

Vaccines and Uses

The differentially expressed nucleic acids and polypeptides produced by the nucleic acids of the invention can also be used to modulate primary immune response to prevent or treat cancer. Every immune response is a complex and intricately regulated sequence of events involving several cell types. It is triggered when an antigen enters the body and encounters a specialized class of cells called antigen-presenting cells (APCs). These APCs capture a minute amount of the antigen and display it in a form that can be recognized by antigen-specific helper T lymphocytes. The helper (Th) cells become activated and, in turn, promote the activation of other classes of lymphocytes, such as B cells or cytotoxic T cells. The activated lymphocytes then proliferate and carry out their specific effector functions, which in many cases successfully activate or eliminate the antigen. Thus, activating the immune response to a particular antigen associated with a cancer cell can protect the patient from developing cancer or result in lymphocytes eliminating cancer cells expressing the antigen.

Gene products, including polypeptides, mRNA (particularly mRNAs having distinct secondary and/or tertiary structures), cDNA, or complete gene, can be prepared and used in vaccines for the treatment or prevention of hyperproliferative disorders and cancers. The nucleic acids and polypeptides can be utilized to enhance the immune response, prevent tumor progression, prevent hyperproliferative cell growth, and the like. Methods for selecting nucleic acids and polypeptides that are capable of enhancing the immune response are known in the art. Preferably, the gene products for use in a vaccine are gene products which are present on the surface of a cell and are recognizable by lymphocytes and antibodies.

The gene products may be formulated with pharmaceutically acceptable carriers into pharmaceutical compositions by methods known in the art. The composition is useful as a vaccine to prevent or treat cancer. The composition may further comprise at least one co-immunostimulatory molecule, including but not limited to one or more major histocompatibility complex (MHC) molecules, such as a class I or class II molecule, preferably a class I molecule. The composition may further comprise other stimulator molecules including B7.1, B7.2, ICAM-1, ICAM-2, LFA-1, LFA-3, CD72 and the like, immunostimulatory polynucleotides (which comprise an 5′-CG-3′ wherein the cytosine is unmethylated), and cytokines which include but are not limited to IL-1 through IL-15, TNF-α, IFN-γ, RANTES, G-CSF, M-CSF, IFN-α, CTAP III, ENA-78, GRO, I-309, PF-4, IP-10, LD-78, MGSA, MIP-1α, MIP-1β, or combination thereof, and the like for immunopotentiation. In one embodiment, the immunopotentiators of particular interest are those that facilitate a Th1 immune response.

The gene products may also be prepared with a carrier that will protect the gene products against rapid elimination from the body, such as a controlled release formulation, including implants and microencapsulated delivery systems. Biodegradable polymers can be used, such as ethylene vinyl acetate, polyanhydrides, polyglycolic acid, collagen, polyorthoesters, polylactic acid, and the like. Methods for preparation of such formulations are known in the art.

In the methods of preventing or treating cancer, the gene products may be administered via one of several routes including but not limited to transdermal, transmucosal, intravenous, intramuscular, subcutaneous, intradermal, intraperitoneal, intrathecal, intrapleural, intrauterine, rectal, vaginal, topical, intratumor, and the like. For transmucosal or transdermal administration, penetrants appropriate to the barrier to be permeated are used in the formulation. Such penetrants are generally known in the art, and include, for example, administration bile salts and fusidic acid derivatives. In addition, detergents may be used to facilitate permeation. Transmucosal administration may be by nasal sprays or suppositories. For oral administration, the gene products are formulated into conventional oral administration form such as capsules, tablets, elixirs and the like.

The gene product is administered to a patient in an amount effective to prevent or treat cancer. In general, it is desirable to provide the patient with a dosage of gene product of at least about 1 pg per Kg body weight, preferably at least about 1 ng per Kg body weight, more preferably at least about 1 μg or greater per Kg body weight of the recipient. A range of from about 1 ng per Kg body weight to about 100 mg per Kg body weight is preferred although a lower or higher dose may be administered. The dose is effective to prime, stimulate and/or cause the clonal expansion of antigen-specific T lymphocytes, preferably cytotoxic T lymphocytes, which in turn are capable of preventing or treating cancer in the recipient. The dose is administered at least once and may be provided as a bolus or a continuous administration. Multiple administrations of the dose over a period of several weeks to months may be preferable. Subsequent doses may be administered as indicated.

In another method of treatment, autologous cytotoxic lymphocytes or tumor infiltrating lymphocytes may be obtained from a patient with cancer. The lymphocytes are grown in culture, and antigen-specific lymphocytes are expanded by culturing in the presence of the specific gene products alone or in combination with at least one co-immunostimulatory molecule with cytokines. The antigen-specific lymphocytes are then infused back into the patient in an amount effective to reduce or eliminate the tumors in the patient. Cancer vaccines and their uses are further described in U.S. Pat. No. 5,961,978; U.S. Pat. No. 5,993,829; U.S. Pat. No. 6,132,980; and WO 00/38706.

Pharmaceutical Compositions and Uses

Pharmaceutical compositions can comprise polypeptides, receptors that specifically bind a polypeptide produced by a differentially expressed gene (e.g., antibodies, or polynucleotides (including antisense nucleotides and ribozymes) of the claimed invention in a therapeutically effective amount. The compositions can be used to treat primary tumors as well as metastases of primary tumors. In addition, the pharmaceutical compositions can be used in conjunction with conventional methods of cancer treatment, e.g., to sensitize tumors to radiation or conventional chemotherapy.

Where the pharmaceutical composition comprises a receptor (such as an antibody) that specifically binds to a gene product encoded by a differentially expressed gene, the receptor can be coupled to a drug for delivery to a treatment site or coupled to a detectable label to facilitate imaging of a site comprising cancer cells. Methods for coupling antibodies to drugs and detectable labels are well known in the art, as are methods for imaging using detectable labels.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

Delivery Methods

Once formulated, the compositions contemplated by the invention can be (1) administered directly to the subject (e.g., as polynucleotide, polypeptides, small molecule agonists or antagonists, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once differential expression of a gene corresponding to a polynucleotide described herein has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents includes local or systemic administration, including injection, oral administration, particle gun or catheterized administration, and topical administration. In general, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of the tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations that will affect the dosage required for ultimate efficacy of the antisense subgenomic polynucleotides.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

The sequences disclosed in this patent application were disclosed in several earlier patent applications. The relationship between the SEQ ID NOS in those earlier application and the SEQ ID NOS disclosed herein is shown in Tables 26 and 27.

TABLE 26
relationship between SEQ ID NOs. this patent application
and SEQ ID NOs of parent patent applications

| | | | | corresponding |
|---|---|---|---|---|
| | parent | | | SEQ IDs in |
| parent | application | | SEQ IDs in | this patent |
| case | no. | filing date | parent case | application |
| 1663 | 09/883,152 | Jun. 15, 2001 | 1-127 | 1-127 |
| 1552CON | 10/165,835 | Jun. 6, 2002 | 1-6 | 128-133 |
| 18178WO | US03/15465 | May 16, 2003 | 1-1548 | 134-1681 |

The disclosures of all prior U.S. applications to which the present application claims priority, which includes those U.S. applications referenced in the table above as well as their respective priority applications, are each incorporated herein by referenced in their entireties for all purposes, including the disclosures found in the Sequence Listings, tables, figures and Examples.

TABLE 27
Lookup table showing corresponding SEQ ID NOS in this
application and parent applications

| | | | corresponding |
|---|---|---|---|
| | parent | parent | SEQ ID NO in |
| SEQ ID NO in | application | application | parent |
| this application | docket no | serial no | application |
| 1 | 2300-1663 | 09/883,152 | 1 |
| 2 | 2300-1663 | 09/883,152 | 2 |
| 3 | 2300-1663 | 09/883,152 | 3 |
| 4 | 2300-1663 | 09/883,152 | 4 |
| 5 | 2300-1663 | 09/883,152 | 5 |
| 6 | 2300-1663 | 09/883,152 | 6 |
| 7 | 2300-1663 | 09/883,152 | 7 |
| 8 | 2300-1663 | 09/883,152 | 8 |
| 9 | 2300-1663 | 09/883,152 | 9 |
| 10 | 2300-1663 | 09/883,152 | 10 |
| 11 | 2300-1663 | 09/883,152 | 11 |
| 12 | 2300-1663 | 09/883,152 | 12 |
| 13 | 2300-1663 | 09/883,152 | 13 |
| 14 | 2300-1663 | 09/883,152 | 14 |
| 15 | 2300-1663 | 09/883,152 | 15 |
| 16 | 2300-1663 | 09/883,152 | 16 |
| 17 | 2300-1663 | 09/883,152 | 17 |
| 18 | 2300-1663 | 09/883,152 | 18 |
| 19 | 2300-1663 | 09/883,152 | 19 |
| 20 | 2300-1663 | 09/883,152 | 20 |
| 21 | 2300-1663 | 09/883,152 | 21 |
| 22 | 2300-1663 | 09/883,152 | 22 |
| 23 | 2300-1663 | 09/883,152 | 23 |
| 24 | 2300-1663 | 09/883,152 | 24 |
| 25 | 2300-1663 | 09/883,152 | 25 |
| 26 | 2300-1663 | 09/883,152 | 26 |
| 27 | 2300-1663 | 09/883,152 | 27 |
| 28 | 2300-1663 | 09/883,152 | 28 |
| 29 | 2300-1663 | 09/883,152 | 29 |
| 30 | 2300-1663 | 09/883,152 | 30 |
| 31 | 2300-1663 | 09/883,152 | 31 |
| 32 | 2300-1663 | 09/883,152 | 32 |
| 33 | 2300-1663 | 09/883,152 | 33 |
| 34 | 2300-1663 | 09/883,152 | 34 |
| 35 | 2300-1663 | 09/883,152 | 35 |
| 36 | 2300-1663 | 09/883,152 | 36 |
| 37 | 2300-1663 | 09/883,152 | 37 |
| 38 | 2300-1663 | 09/883,152 | 38 |
| 39 | 2300-1663 | 09/883,152 | 39 |
| 40 | 2300-1663 | 09/883,152 | 40 |
| 41 | 2300-1663 | 09/883,152 | 41 |
| 42 | 2300-1663 | 09/883,152 | 42 |
| 43 | 2300-1663 | 09/883,152 | 43 |
| 44 | 2300-1663 | 09/883,152 | 44 |
| 45 | 2300-1663 | 09/883,152 | 45 |
| 46 | 2300-1663 | 09/883,152 | 46 |
| 47 | 2300-1663 | 09/883,152 | 47 |
| 48 | 2300-1663 | 09/883,152 | 48 |
| 49 | 2300-1663 | 09/883,152 | 49 |
| 50 | 2300-1663 | 09/883,152 | 50 |
| 51 | 2300-1663 | 09/883,152 | 51 |
| 52 | 2300-1663 | 09/883,152 | 52 |
| 53 | 2300-1663 | 09/883,152 | 53 |
| 54 | 2300-1663 | 09/883,152 | 54 |
| 55 | 2300-1663 | 09/883,152 | 55 |
| 56 | 2300-1663 | 09/883,152 | 56 |
| 57 | 2300-1663 | 09/883,152 | 57 |
| 58 | 2300-1663 | 09/883,152 | 58 |
| 59 | 2300-1663 | 09/883,152 | 59 |
| 60 | 2300-1663 | 09/883,152 | 60 |
| 61 | 2300-1663 | 09/883,152 | 61 |
| 62 | 2300-1663 | 09/883,152 | 62 |
| 63 | 2300-1663 | 09/883,152 | 63 |
| 64 | 2300-1663 | 09/883,152 | 64 |
| 65 | 2300-1663 | 09/883,152 | 65 |
| 66 | 2300-1663 | 09/883,152 | 66 |
| 67 | 2300-1663 | 09/883,152 | 67 |
| 68 | 2300-1663 | 09/883,152 | 68 |
| 69 | 2300-1663 | 09/883,152 | 69 |
| 70 | 2300-1663 | 09/883,152 | 70 |
| 71 | 2300-1663 | 09/883,152 | 71 |
| 72 | 2300-1663 | 09/883,152 | 72 |
| 73 | 2300-1663 | 09/883,152 | 73 |
| 74 | 2300-1663 | 09/883,152 | 74 |
| 75 | 2300-1663 | 09/883,152 | 75 |
| 76 | 2300-1663 | 09/883,152 | 76 |
| 77 | 2300-1663 | 09/883,152 | 77 |
| 78 | 2300-1663 | 09/883,152 | 78 |
| 79 | 2300-1663 | 09/883,152 | 79 |
| 80 | 2300-1663 | 09/883,152 | 80 |
| 81 | 2300-1663 | 09/883,152 | 81 |
| 82 | 2300-1663 | 09/883,152 | 82 |
| 83 | 2300-1663 | 09/883,152 | 83 |
| 84 | 2300-1663 | 09/883,152 | 84 |
| 85 | 2300-1663 | 09/883,152 | 85 |
| 86 | 2300-1663 | 09/883,152 | 86 |
| 87 | 2300-1663 | 09/883,152 | 87 |
| 88 | 2300-1663 | 09/883,152 | 88 |
| 89 | 2300-1663 | 09/883,152 | 89 |
| 90 | 2300-1663 | 09/883,152 | 90 |
| 91 | 2300-1663 | 09/883,152 | 91 |
| 92 | 2300-1663 | 09/883,152 | 92 |
| 93 | 2300-1663 | 09/883,152 | 93 |
| 94 | 2300-1663 | 09/883,152 | 94 |
| 95 | 2300-1663 | 09/883,152 | 95 |
| 96 | 2300-1663 | 09/883,152 | 96 |
| 97 | 2300-1663 | 09/883,152 | 97 |
| 98 | 2300-1663 | 09/883,152 | 98 |
| 99 | 2300-1663 | 09/883,152 | 99 |
| 100 | 2300-1663 | 09/883,152 | 100 |
| 101 | 2300-1663 | 09/883,152 | 101 |
| 102 | 2300-1663 | 09/883,152 | 102 |
| 103 | 2300-1663 | 09/883,152 | 103 |
| 104 | 2300-1663 | 09/883,152 | 104 |
| 105 | 2300-1663 | 09/883,152 | 105 |
| 106 | 2300-1663 | 09/883,152 | 106 |
| 107 | 2300-1663 | 09/883,152 | 107 |
| 108 | 2300-1663 | 09/883,152 | 108 |
| 109 | 2300-1663 | 09/883,152 | 109 |
| 110 | 2300-1663 | 09/883,152 | 110 |
| 111 | 2300-1663 | 09/883,152 | 111 |
| 112 | 2300-1663 | 09/883,152 | 112 |
| 113 | 2300-1663 | 09/883,152 | 113 |
| 114 | 2300-1663 | 09/883,152 | 114 |
| 115 | 2300-1663 | 09/883,152 | 115 |
| 116 | 2300-1663 | 09/883,152 | 116 |
| 117 | 2300-1663 | 09/883,152 | 117 |
| 118 | 2300-1663 | 09/883,152 | 118 |
| 119 | 2300-1663 | 09/883,152 | 119 |
| 120 | 2300-1663 | 09/883,152 | 120 |
| 121 | 2300-1663 | 09/883,152 | 121 |
| 122 | 2300-1663 | 09/883,152 | 122 |
| 123 | 2300-1663 | 09/883,152 | 123 |
| 124 | 2300-1663 | 09/883,152 | 124 |
| 125 | 2300-1663 | 09/883,152 | 125 |
| 126 | 2300-1663 | 09/883,152 | 126 |
| 127 | 2300-1663 | 09/883,152 | 127 |
| 128 | 2300-1552CON | 10/165,835 | 1 |
| 129 | 2300-1552CON | 10/165,835 | 2 |
| 130 | 2300-1552CON | 10/165,835 | 3 |
| 131 | 2300-1552CON | 10/165,835 | 4 |
| 132 | 2300-1552CON | 10/165,835 | 5 |
| 133 | 2300-1552CON | 10/165,835 | 6 |
| 134 | 2300-18178WO | US03/15465 | 1 |
| 135 | 2300-18178WO | US03/15465 | 2 |
| 136 | 2300-18178WO | US03/15465 | 3 |
| 137 | 2300-18178WO | US03/15465 | 4 |
| 138 | 2300-18178WO | US03/15465 | 5 |
| 139 | 2300-18178WO | US03/15465 | 6 |
| 140 | 2300-18178WO | US03/15465 | 7 |
| 141 | 2300-18178WO | US03/15465 | 8 |
| 142 | 2300-18178WO | US03/15465 | 9 |
| 143 | 2300-18178WO | US03/15465 | 10 |
| 144 | 2300-18178WO | US03/15465 | 11 |
| 145 | 2300-18178WO | US03/15465 | 12 |
| 146 | 2300-18178WO | US03/15465 | 13 |
| 147 | 2300-18178WO | US03/15465 | 14 |
| 148 | 2300-18178WO | US03/15465 | 15 |
| 149 | 2300-18178WO | US03/15465 | 16 |
| 150 | 2300-18178WO | US03/15465 | 17 |
| 151 | 2300-18178WO | US03/15465 | 18 |
| 152 | 2300-18178WO | US03/15465 | 19 |
| 153 | 2300-18178WO | US03/15465 | 20 |
| 154 | 2300-18178WO | US03/15465 | 21 |
| 155 | 2300-18178WO | US03/15465 | 22 |
| 156 | 2300-18178WO | US03/15465 | 23 |
| 157 | 2300-18178WO | US03/15465 | 24 |
| 158 | 2300-18178WO | US03/15465 | 25 |
| 159 | 2300-18178WO | US03/15465 | 26 |
| 160 | 2300-18178WO | US03/15465 | 27 |
| 161 | 2300-18178WO | US03/15465 | 28 |
| 162 | 2300-18178WO | US03/15465 | 29 |
| 163 | 2300-18178WO | US03/15465 | 30 |
| 164 | 2300-18178WO | US03/15465 | 31 |
| 165 | 2300-18178WO | US03/15465 | 32 |
| 166 | 2300-18178WO | US03/15465 | 33 |
| 167 | 2300-18178WO | US03/15465 | 34 |
| 168 | 2300-18178WO | US03/15465 | 35 |
| 169 | 2300-18178WO | US03/15465 | 36 |
| 170 | 2300-18178WO | US03/15465 | 37 |
| 171 | 2300-18178WO | US03/15465 | 38 |
| 172 | 2300-18178WO | US03/15465 | 39 |
| 173 | 2300-18178WO | US03/15465 | 40 |
| 174 | 2300-18178WO | US03/15465 | 41 |
| 175 | 2300-18178WO | US03/15465 | 42 |
| 176 | 2300-18178WO | US03/15465 | 43 |
| 177 | 2300-18178WO | US03/15465 | 44 |
| 178 | 2300-18178WO | US03/15465 | 45 |
| 179 | 2300-18178WO | US03/15465 | 46 |
| 180 | 2300-18178WO | US03/15465 | 47 |
| 181 | 2300-18178WO | US03/15465 | 48 |
| 182 | 2300-18178WO | US03/15465 | 49 |
| 183 | 2300-18178WO | US03/15465 | 50 |
| 184 | 2300-18178WO | US03/15465 | 51 |
| 185 | 2300-18178WO | US03/15465 | 52 |
| 186 | 2300-18178WO | US03/15465 | 53 |
| 187 | 2300-18178WO | US03/15465 | 54 |
| 188 | 2300-18178WO | US03/15465 | 55 |
| 189 | 2300-18178WO | US03/15465 | 56 |
| 190 | 2300-18178WO | US03/15465 | 57 |
| 191 | 2300-18178WO | US03/15465 | 58 |
| 192 | 2300-18178WO | US03/15465 | 59 |
| 193 | 2300-18178WO | US03/15465 | 60 |
| 194 | 2300-18178WO | US03/15465 | 61 |
| 195 | 2300-18178WO | US03/15465 | 62 |
| 196 | 2300-18178WO | US03/15465 | 63 |
| 197 | 2300-18178WO | US03/15465 | 64 |
| 198 | 2300-18178WO | US03/15465 | 65 |
| 199 | 2300-18178WO | US03/15465 | 66 |
| 200 | 2300-18178WO | US03/15465 | 67 |
| 201 | 2300-18178WO | US03/15465 | 68 |
| 202 | 2300-18178WO | US03/15465 | 69 |
| 203 | 2300-18178WO | US03/15465 | 70 |
| 204 | 2300-18178WO | US03/15465 | 71 |
| 205 | 2300-18178WO | US03/15465 | 72 |
| 206 | 2300-18178WO | US03/15465 | 73 |
| 207 | 2300-18178WO | US03/15465 | 74 |
| 208 | 2300-18178WO | US03/15465 | 75 |
| 209 | 2300-18178WO | US03/15465 | 76 |
| 210 | 2300-18178WO | US03/15465 | 77 |
| 211 | 2300-18178WO | US03/15465 | 78 |
| 212 | 2300-18178WO | US03/15465 | 79 |
| 213 | 2300-18178WO | US03/15465 | 80 |
| 214 | 2300-18178WO | US03/15465 | 81 |
| 215 | 2300-18178WO | US03/15465 | 82 |
| 216 | 2300-18178WO | US03/15465 | 83 |
| 217 | 2300-18178WO | US03/15465 | 84 |
| 218 | 2300-18178WO | US03/15465 | 85 |
| 219 | 2300-18178WO | US03/15465 | 86 |
| 220 | 2300-18178WO | US03/15465 | 87 |
| 221 | 2300-18178WO | US03/15465 | 88 |
| 222 | 2300-18178WO | US03/15465 | 89 |
| 223 | 2300-18178WO | US03/15465 | 90 |
| 224 | 2300-18178WO | US03/15465 | 91 |
| 225 | 2300-18178WO | US03/15465 | 92 |
| 226 | 2300-18178WO | US03/15465 | 93 |
| 227 | 2300-18178WO | US03/15465 | 94 |
| 228 | 2300-18178WO | US03/15465 | 95 |
| 229 | 2300-18178WO | US03/15465 | 96 |
| 230 | 2300-18178WO | US03/15465 | 97 |
| 231 | 2300-18178WO | US03/15465 | 98 |
| 232 | 2300-18178WO | US03/15465 | 99 |
| 233 | 2300-18178WO | US03/15465 | 100 |
| 234 | 2300-18178WO | US03/15465 | 101 |
| 235 | 2300-18178WO | US03/15465 | 102 |
| 236 | 2300-18178WO | US03/15465 | 103 |
| 237 | 2300-18178WO | US03/15465 | 104 |
| 238 | 2300-18178WO | US03/15465 | 105 |
| 239 | 2300-18178WO | US03/15465 | 106 |
| 240 | 2300-18178WO | US03/15465 | 107 |
| 241 | 2300-18178WO | US03/15465 | 108 |
| 242 | 2300-18178WO | US03/15465 | 109 |
| 243 | 2300-18178WO | US03/15465 | 110 |
| 244 | 2300-18178WO | US03/15465 | 111 |
| 245 | 2300-18178WO | US03/15465 | 112 |
| 246 | 2300-18178WO | US03/15465 | 113 |
| 247 | 2300-18178WO | US03/15465 | 114 |
| 248 | 2300-18178WO | US03/15465 | 115 |
| 249 | 2300-18178WO | US03/15465 | 116 |
| 250 | 2300-18178WO | US03/15465 | 117 |
| 251 | 2300-18178WO | US03/15465 | 118 |
| 252 | 2300-18178WO | US03/15465 | 119 |
| 253 | 2300-18178WO | US03/15465 | 120 |
| 254 | 2300-18178WO | US03/15465 | 121 |
| 255 | 2300-18178WO | US03/15465 | 122 |
| 256 | 2300-18178WO | US03/15465 | 123 |
| 257 | 2300-18178WO | US03/15465 | 124 |
| 258 | 2300-18178WO | US03/15465 | 125 |
| 259 | 2300-18178WO | US03/15465 | 126 |
| 260 | 2300-18178WO | US03/15465 | 127 |
| 261 | 2300-18178WO | US03/15465 | 128 |
| 262 | 2300-18178WO | US03/15465 | 129 |
| 263 | 2300-18178WO | US03/15465 | 130 |
| 264 | 2300-18178WO | US03/15465 | 131 |
| 265 | 2300-18178WO | US03/15465 | 132 |
| 266 | 2300-18178WO | US03/15465 | 133 |
| 267 | 2300-18178WO | US03/15465 | 134 |
| 268 | 2300-18178WO | US03/15465 | 135 |
| 269 | 2300-18178WO | US03/15465 | 136 |
| 270 | 2300-18178WO | US03/15465 | 137 |
| 271 | 2300-18178WO | US03/15465 | 138 |
| 272 | 2300-18178WO | US03/15465 | 139 |
| 273 | 2300-18178WO | US03/15465 | 140 |
| 274 | 2300-18178WO | US03/15465 | 141 |
| 275 | 2300-18178WO | US03/15465 | 142 |
| 276 | 2300-18178WO | US03/15465 | 143 |
| 277 | 2300-18178WO | US03/15465 | 144 |
| 278 | 2300-18178WO | US03/15465 | 145 |
| 279 | 2300-18178WO | US03/15465 | 146 |
| 280 | 2300-18178WO | US03/15465 | 147 |
| 281 | 2300-18178WO | US03/15465 | 148 |
| 282 | 2300-18178WO | US03/15465 | 149 |
| 283 | 2300-18178WO | US03/15465 | 150 |
| 284 | 2300-18178WO | US03/15465 | 151 |
| 285 | 2300-18178WO | US03/15465 | 152 |
| 286 | 2300-18178WO | US03/15465 | 153 |
| 287 | 2300-18178WO | US03/15465 | 154 |
| 288 | 2300-18178WO | US03/15465 | 155 |
| 289 | 2300-18178WO | US03/15465 | 156 |
| 290 | 2300-18178WO | US03/15465 | 157 |
| 291 | 2300-18178WO | US03/15465 | 158 |
| 292 | 2300-18178WO | US03/15465 | 159 |
| 293 | 2300-18178WO | US03/15465 | 160 |
| 294 | 2300-18178WO | US03/15465 | 161 |
| 295 | 2300-18178WO | US03/15465 | 162 |
| 296 | 2300-18178WO | US03/15465 | 163 |
| 297 | 2300-18178WO | US03/15465 | 164 |
| 298 | 2300-18178WO | US03/15465 | 165 |
| 299 | 2300-18178WO | US03/15465 | 166 |
| 300 | 2300-18178WO | US03/15465 | 167 |
| 301 | 2300-18178WO | US03/15465 | 168 |
| 302 | 2300-18178WO | US03/15465 | 169 |
| 303 | 2300-18178WO | US03/15465 | 170 |
| 304 | 2300-18178WO | US03/15465 | 171 |
| 305 | 2300-18178WO | US03/15465 | 172 |
| 306 | 2300-18178WO | US03/15465 | 173 |
| 307 | 2300-18178WO | US03/15465 | 174 |
| 308 | 2300-18178WO | US03/15465 | 175 |
| 309 | 2300-18178WO | US03/15465 | 176 |
| 310 | 2300-18178WO | US03/15465 | 177 |
| 311 | 2300-18178WO | US03/15465 | 178 |
| 312 | 2300-18178WO | US03/15465 | 179 |
| 313 | 2300-18178WO | US03/15465 | 180 |
| 314 | 2300-18178WO | US03/15465 | 181 |
| 315 | 2300-18178WO | US03/15465 | 182 |
| 316 | 2300-18178WO | US03/15465 | 183 |
| 317 | 2300-18178WO | US03/15465 | 184 |
| 318 | 2300-18178WO | US03/15465 | 185 |
| 319 | 2300-18178WO | US03/15465 | 186 |
| 320 | 2300-18178WO | US03/15465 | 187 |
| 321 | 2300-18178WO | US03/15465 | 188 |
| 322 | 2300-18178WO | US03/15465 | 189 |
| 323 | 2300-18178WO | US03/15465 | 190 |
| 324 | 2300-18178WO | US03/15465 | 191 |
| 325 | 2300-18178WO | US03/15465 | 192 |
| 326 | 2300-18178WO | US03/15465 | 193 |
| 327 | 2300-18178WO | US03/15465 | 194 |
| 328 | 2300-18178WO | US03/15465 | 195 |
| 329 | 2300-18178WO | US03/15465 | 196 |
| 330 | 2300-18178WO | US03/15465 | 197 |
| 331 | 2300-18178WO | US03/15465 | 198 |
| 332 | 2300-18178WO | US03/15465 | 199 |
| 333 | 2300-18178WO | US03/15465 | 200 |
| 334 | 2300-18178WO | US03/15465 | 201 |
| 335 | 2300-18178WO | US03/15465 | 202 |
| 336 | 2300-18178WO | US03/15465 | 203 |
| 337 | 2300-18178WO | US03/15465 | 204 |
| 338 | 2300-18178WO | US03/15465 | 205 |
| 339 | 2300-18178WO | US03/15465 | 206 |
| 340 | 2300-18178WO | US03/15465 | 207 |
| 341 | 2300-18178WO | US03/15465 | 208 |
| 342 | 2300-18178WO | US03/15465 | 209 |
| 343 | 2300-18178WO | US03/15465 | 210 |
| 344 | 2300-18178WO | US03/15465 | 211 |
| 345 | 2300-18178WO | US03/15465 | 212 |
| 346 | 2300-18178WO | US03/15465 | 213 |
| 347 | 2300-18178WO | US03/15465 | 214 |
| 348 | 2300-18178WO | US03/15465 | 215 |
| 349 | 2300-18178WO | US03/15465 | 216 |
| 350 | 2300-18178WO | US03/15465 | 217 |
| 351 | 2300-18178WO | US03/15465 | 218 |
| 352 | 2300-18178WO | US03/15465 | 219 |
| 353 | 2300-18178WO | US03/15465 | 220 |
| 354 | 2300-18178WO | US03/15465 | 221 |
| 355 | 2300-18178WO | US03/15465 | 222 |
| 356 | 2300-18178WO | US03/15465 | 223 |
| 357 | 2300-18178WO | US03/15465 | 224 |
| 358 | 2300-18178WO | US03/15465 | 225 |
| 359 | 2300-18178WO | US03/15465 | 226 |
| 360 | 2300-18178WO | US03/15465 | 227 |
| 361 | 2300-18178WO | US03/15465 | 228 |
| 362 | 2300-18178WO | US03/15465 | 229 |
| 363 | 2300-18178WO | US03/15465 | 230 |
| 364 | 2300-18178WO | US03/15465 | 231 |
| 365 | 2300-18178WO | US03/15465 | 232 |
| 366 | 2300-18178WO | US03/15465 | 233 |
| 367 | 2300-18178WO | US03/15465 | 234 |
| 368 | 2300-18178WO | US03/15465 | 235 |
| 369 | 2300-18178WO | US03/15465 | 236 |
| 370 | 2300-18178WO | US03/15465 | 237 |
| 371 | 2300-18178WO | US03/15465 | 238 |
| 372 | 2300-18178WO | US03/15465 | 239 |
| 373 | 2300-18178WO | US03/15465 | 240 |
| 374 | 2300-18178WO | US03/15465 | 241 |
| 375 | 2300-18178WO | US03/15465 | 242 |
| 376 | 2300-18178WO | US03/15465 | 243 |
| 377 | 2300-18178WO | US03/15465 | 244 |
| 378 | 2300-18178WO | US03/15465 | 245 |
| 379 | 2300-18178WO | US03/15465 | 246 |
| 380 | 2300-18178WO | US03/15465 | 247 |
| 381 | 2300-18178WO | US03/15465 | 248 |
| 382 | 2300-18178WO | US03/15465 | 249 |
| 383 | 2300-18178WO | US03/15465 | 250 |
| 384 | 2300-18178WO | US03/15465 | 251 |
| 385 | 2300-18178WO | US03/15465 | 252 |
| 386 | 2300-18178WO | US03/15465 | 253 |
| 387 | 2300-18178WO | US03/15465 | 254 |
| 388 | 2300-18178WO | US03/15465 | 255 |
| 389 | 2300-18178WO | US03/15465 | 256 |
| 390 | 2300-18178WO | US03/15465 | 257 |
| 391 | 2300-18178WO | US03/15465 | 258 |
| 392 | 2300-18178WO | US03/15465 | 259 |
| 393 | 2300-18178WO | US03/15465 | 260 |
| 394 | 2300-18178WO | US03/15465 | 261 |
| 395 | 2300-18178WO | US03/15465 | 262 |
| 396 | 2300-18178WO | US03/15465 | 263 |
| 397 | 2300-18178WO | US03/15465 | 264 |
| 398 | 2300-18178WO | US03/15465 | 265 |
| 399 | 2300-18178WO | US03/15465 | 266 |
| 400 | 2300-18178WO | US03/15465 | 267 |
| 401 | 2300-18178WO | US03/15465 | 268 |
| 402 | 2300-18178WO | US03/15465 | 269 |
| 403 | 2300-18178WO | US03/15465 | 270 |
| 404 | 2300-18178WO | US03/15465 | 271 |
| 405 | 2300-18178WO | US03/15465 | 272 |
| 406 | 2300-18178WO | US03/15465 | 273 |
| 407 | 2300-18178WO | US03/15465 | 274 |
| 408 | 2300-18178WO | US03/15465 | 275 |
| 409 | 2300-18178WO | US03/15465 | 276 |
| 410 | 2300-18178WO | US03/15465 | 277 |
| 411 | 2300-18178WO | US03/15465 | 278 |
| 412 | 2300-18178WO | US03/15465 | 279 |
| 413 | 2300-18178WO | US03/15465 | 280 |
| 414 | 2300-18178WO | US03/15465 | 281 |
| 415 | 2300-18178WO | US03/15465 | 282 |
| 416 | 2300-18178WO | US03/15465 | 283 |
| 417 | 2300-18178WO | US03/15465 | 284 |
| 418 | 2300-18178WO | US03/15465 | 285 |
| 419 | 2300-18178WO | US03/15465 | 286 |
| 420 | 2300-18178WO | US03/15465 | 287 |
| 421 | 2300-18178WO | US03/15465 | 288 |
| 422 | 2300-18178WO | US03/15465 | 289 |
| 423 | 2300-18178WO | US03/15465 | 290 |
| 424 | 2300-18178WO | US03/15465 | 291 |
| 425 | 2300-18178WO | US03/15465 | 292 |
| 426 | 2300-18178WO | US03/15465 | 293 |
| 427 | 2300-18178WO | US03/15465 | 294 |
| 428 | 2300-18178WO | US03/15465 | 295 |
| 429 | 2300-18178WO | US03/15465 | 296 |
| 430 | 2300-18178WO | US03/15465 | 297 |
| 431 | 2300-18178WO | US03/15465 | 298 |
| 432 | 2300-18178WO | US03/15465 | 299 |
| 433 | 2300-18178WO | US03/15465 | 300 |
| 434 | 2300-18178WO | US03/15465 | 301 |
| 435 | 2300-18178WO | US03/15465 | 302 |
| 436 | 2300-18178WO | US03/15465 | 303 |
| 437 | 2300-18178WO | US03/15465 | 304 |
| 438 | 2300-18178WO | US03/15465 | 305 |
| 439 | 2300-18178WO | US03/15465 | 306 |
| 440 | 2300-18178WO | US03/15465 | 307 |
| 441 | 2300-18178WO | US03/15465 | 308 |
| 442 | 2300-18178WO | US03/15465 | 309 |
| 443 | 2300-18178WO | US03/15465 | 310 |
| 444 | 2300-18178WO | US03/15465 | 311 |
| 445 | 2300-18178WO | US03/15465 | 312 |
| 446 | 2300-18178WO | US03/15465 | 313 |
| 447 | 2300-18178WO | US03/15465 | 314 |
| 448 | 2300-18178WO | US03/15465 | 315 |
| 449 | 2300-18178WO | US03/15465 | 316 |
| 450 | 2300-18178WO | US03/15465 | 317 |
| 451 | 2300-18178WO | US03/15465 | 318 |
| 452 | 2300-18178WO | US03/15465 | 319 |
| 453 | 2300-18178WO | US03/15465 | 320 |
| 454 | 2300-18178WO | US03/15465 | 321 |
| 455 | 2300-18178WO | US03/15465 | 322 |
| 456 | 2300-18178WO | US03/15465 | 323 |
| 457 | 2300-18178WO | US03/15465 | 324 |
| 458 | 2300-18178WO | US03/15465 | 325 |
| 459 | 2300-18178WO | US03/15465 | 326 |
| 460 | 2300-18178WO | US03/15465 | 327 |
| 461 | 2300-18178WO | US03/15465 | 328 |
| 462 | 2300-18178WO | US03/15465 | 329 |
| 463 | 2300-18178WO | US03/15465 | 330 |
| 464 | 2300-18178WO | US03/15465 | 331 |
| 465 | 2300-18178WO | US03/15465 | 332 |
| 466 | 2300-18178WO | US03/15465 | 333 |
| 467 | 2300-18178WO | US03/15465 | 334 |
| 468 | 2300-18178WO | US03/15465 | 335 |
| 469 | 2300-18178WO | US03/15465 | 336 |
| 470 | 2300-18178WO | US03/15465 | 337 |
| 471 | 2300-18178WO | US03/15465 | 338 |
| 472 | 2300-18178WO | US03/15465 | 339 |
| 473 | 2300-18178WO | US03/15465 | 340 |
| 474 | 2300-18178WO | US03/15465 | 341 |
| 475 | 2300-18178WO | US03/15465 | 342 |
| 476 | 2300-18178WO | US03/15465 | 343 |
| 477 | 2300-18178WO | US03/15465 | 344 |
| 478 | 2300-18178WO | US03/15465 | 345 |
| 479 | 2300-18178WO | US03/15465 | 346 |
| 480 | 2300-18178WO | US03/15465 | 347 |
| 481 | 2300-18178WO | US03/15465 | 348 |
| 482 | 2300-18178WO | US03/15465 | 349 |
| 483 | 2300-18178WO | US03/15465 | 350 |
| 484 | 2300-18178WO | US03/15465 | 351 |
| 485 | 2300-18178WO | US03/15465 | 352 |
| 486 | 2300-18178WO | US03/15465 | 353 |
| 487 | 2300-18178WO | US03/15465 | 354 |
| 488 | 2300-18178WO | US03/15465 | 355 |
| 489 | 2300-18178WO | US03/15465 | 356 |
| 490 | 2300-18178WO | US03/15465 | 357 |
| 491 | 2300-18178WO | US03/15465 | 358 |
| 492 | 2300-18178WO | US03/15465 | 359 |
| 493 | 2300-18178WO | US03/15465 | 360 |
| 494 | 2300-18178WO | US03/15465 | 361 |
| 495 | 2300-18178WO | US03/15465 | 362 |
| 496 | 2300-18178WO | US03/15465 | 363 |
| 497 | 2300-18178WO | US03/15465 | 364 |
| 498 | 2300-18178WO | US03/15465 | 365 |
| 499 | 2300-18178WO | US03/15465 | 366 |
| 500 | 2300-18178WO | US03/15465 | 367 |
| 501 | 2300-18178WO | US03/15465 | 368 |
| 502 | 2300-18178WO | US03/15465 | 369 |
| 503 | 2300-18178WO | US03/15465 | 370 |
| 504 | 2300-18178WO | US03/15465 | 371 |
| 505 | 2300-18178WO | US03/15465 | 372 |
| 506 | 2300-18178WO | US03/15465 | 373 |
| 507 | 2300-18178WO | US03/15465 | 374 |
| 508 | 2300-18178WO | US03/15465 | 375 |
| 509 | 2300-18178WO | US03/15465 | 376 |
| 510 | 2300-18178WO | US03/15465 | 377 |
| 511 | 2300-18178WO | US03/15465 | 378 |
| 512 | 2300-18178WO | US03/15465 | 379 |
| 513 | 2300-18178WO | US03/15465 | 380 |
| 514 | 2300-18178WO | US03/15465 | 381 |
| 515 | 2300-18178WO | US03/15465 | 382 |
| 516 | 2300-18178WO | US03/15465 | 383 |
| 517 | 2300-18178WO | US03/15465 | 384 |
| 518 | 2300-18178WO | US03/15465 | 385 |
| 519 | 2300-18178WO | US03/15465 | 386 |
| 520 | 2300-18178WO | US03/15465 | 387 |
| 521 | 2300-18178WO | US03/15465 | 388 |
| 522 | 2300-18178WO | US03/15465 | 389 |
| 523 | 2300-18178WO | US03/15465 | 390 |
| 524 | 2300-18178WO | US03/15465 | 391 |
| 525 | 2300-18178WO | US03/15465 | 392 |
| 526 | 2300-18178WO | US03/15465 | 393 |
| 527 | 2300-18178WO | US03/15465 | 394 |
| 528 | 2300-18178WO | US03/15465 | 395 |
| 529 | 2300-18178WO | US03/15465 | 396 |
| 530 | 2300-18178WO | US03/15465 | 397 |
| 531 | 2300-18178WO | US03/15465 | 398 |
| 532 | 2300-18178WO | US03/15465 | 399 |
| 533 | 2300-18178WO | US03/15465 | 400 |
| 534 | 2300-18178WO | US03/15465 | 401 |
| 535 | 2300-18178WO | US03/15465 | 402 |
| 536 | 2300-18178WO | US03/15465 | 403 |
| 537 | 2300-18178WO | US03/15465 | 404 |
| 538 | 2300-18178WO | US03/15465 | 405 |
| 539 | 2300-18178WO | US03/15465 | 406 |
| 540 | 2300-18178WO | US03/15465 | 407 |
| 541 | 2300-18178WO | US03/15465 | 408 |
| 542 | 2300-18178WO | US03/15465 | 409 |
| 543 | 2300-18178WO | US03/15465 | 410 |
| 544 | 2300-18178WO | US03/15465 | 411 |
| 545 | 2300-18178WO | US03/15465 | 412 |
| 546 | 2300-18178WO | US03/15465 | 413 |
| 547 | 2300-18178WO | US03/15465 | 414 |
| 548 | 2300-18178WO | US03/15465 | 415 |
| 549 | 2300-18178WO | US03/15465 | 416 |
| 550 | 2300-18178WO | US03/15465 | 417 |
| 551 | 2300-18178WO | US03/15465 | 418 |
| 552 | 2300-18178WO | US03/15465 | 419 |
| 553 | 2300-18178WO | US03/15465 | 420 |
| 554 | 2300-18178WO | US03/15465 | 421 |
| 555 | 2300-18178WO | US03/15465 | 422 |
| 556 | 2300-18178WO | US03/15465 | 423 |
| 557 | 2300-18178WO | US03/15465 | 424 |
| 558 | 2300-18178WO | US03/15465 | 425 |
| 559 | 2300-18178WO | US03/15465 | 426 |
| 560 | 2300-18178WO | US03/15465 | 427 |
| 561 | 2300-18178WO | US03/15465 | 428 |
| 562 | 2300-18178WO | US03/15465 | 429 |
| 563 | 2300-18178WO | US03/15465 | 430 |
| 564 | 2300-18178WO | US03/15465 | 431 |
| 565 | 2300-18178WO | US03/15465 | 432 |
| 566 | 2300-18178WO | US03/15465 | 433 |
| 567 | 2300-18178WO | US03/15465 | 434 |
| 568 | 2300-18178WO | US03/15465 | 435 |
| 569 | 2300-18178WO | US03/15465 | 436 |
| 570 | 2300-18178WO | US03/15465 | 437 |
| 571 | 2300-18178WO | US03/15465 | 438 |
| 572 | 2300-18178WO | US03/15465 | 439 |
| 573 | 2300-18178WO | US03/15465 | 440 |
| 574 | 2300-18178WO | US03/15465 | 441 |
| 575 | 2300-18178WO | US03/15465 | 442 |
| 576 | 2300-18178WO | US03/15465 | 443 |
| 577 | 2300-18178WO | US03/15465 | 444 |
| 578 | 2300-18178WO | US03/15465 | 445 |
| 579 | 2300-18178WO | US03/15465 | 446 |
| 580 | 2300-18178WO | US03/15465 | 447 |
| 581 | 2300-18178WO | US03/15465 | 448 |
| 582 | 2300-18178WO | US03/15465 | 449 |
| 583 | 2300-18178WO | US03/15465 | 450 |
| 584 | 2300-18178WO | US03/15465 | 451 |
| 585 | 2300-18178WO | US03/15465 | 452 |
| 586 | 2300-18178WO | US03/15465 | 453 |
| 587 | 2300-18178WO | US03/15465 | 454 |
| 588 | 2300-18178WO | US03/15465 | 455 |
| 589 | 2300-18178WO | US03/15465 | 456 |
| 590 | 2300-18178WO | US03/15465 | 457 |
| 591 | 2300-18178WO | US03/15465 | 458 |
| 592 | 2300-18178WO | US03/15465 | 459 |
| 593 | 2300-18178WO | US03/15465 | 460 |
| 594 | 2300-18178WO | US03/15465 | 461 |
| 595 | 2300-18178WO | US03/15465 | 462 |
| 596 | 2300-18178WO | US03/15465 | 463 |
| 597 | 2300-18178WO | US03/15465 | 464 |
| 598 | 2300-18178WO | US03/15465 | 465 |
| 599 | 2300-18178WO | US03/15465 | 466 |
| 600 | 2300-18178WO | US03/15465 | 467 |
| 601 | 2300-18178WO | US03/15465 | 468 |
| 602 | 2300-18178WO | US03/15465 | 469 |
| 603 | 2300-18178WO | US03/15465 | 470 |
| 604 | 2300-18178WO | US03/15465 | 471 |
| 605 | 2300-18178WO | US03/15465 | 472 |
| 606 | 2300-18178WO | US03/15465 | 473 |
| 607 | 2300-18178WO | US03/15465 | 474 |
| 608 | 2300-18178WO | US03/15465 | 475 |
| 609 | 2300-18178WO | US03/15465 | 476 |
| 610 | 2300-18178WO | US03/15465 | 477 |
| 611 | 2300-18178WO | US03/15465 | 478 |
| 612 | 2300-18178WO | US03/15465 | 479 |
| 613 | 2300-18178WO | US03/15465 | 480 |
| 614 | 2300-18178WO | US03/15465 | 481 |
| 615 | 2300-18178WO | US03/15465 | 482 |
| 616 | 2300-18178WO | US03/15465 | 483 |
| 617 | 2300-18178WO | US03/15465 | 484 |
| 618 | 2300-18178WO | US03/15465 | 485 |
| 619 | 2300-18178WO | US03/15465 | 486 |
| 620 | 2300-18178WO | US03/15465 | 487 |
| 621 | 2300-18178WO | US03/15465 | 488 |
| 622 | 2300-18178WO | US03/15465 | 489 |
| 623 | 2300-18178WO | US03/15465 | 490 |
| 624 | 2300-18178WO | US03/15465 | 491 |
| 625 | 2300-18178WO | US03/15465 | 492 |
| 626 | 2300-18178WO | US03/15465 | 493 |
| 627 | 2300-18178WO | US03/15465 | 494 |
| 628 | 2300-18178WO | US03/15465 | 495 |
| 629 | 2300-18178WO | US03/15465 | 496 |
| 630 | 2300-18178WO | US03/15465 | 497 |
| 631 | 2300-18178WO | US03/15465 | 498 |
| 632 | 2300-18178WO | US03/15465 | 499 |
| 633 | 2300-18178WO | US03/15465 | 500 |
| 634 | 2300-18178WO | US03/15465 | 501 |
| 635 | 2300-18178WO | US03/15465 | 502 |
| 636 | 2300-18178WO | US03/15465 | 503 |
| 637 | 2300-18178WO | US03/15465 | 504 |
| 638 | 2300-18178WO | US03/15465 | 505 |
| 639 | 2300-18178WO | US03/15465 | 506 |
| 640 | 2300-18178WO | US03/15465 | 507 |
| 641 | 2300-18178WO | US03/15465 | 508 |
| 642 | 2300-18178WO | US03/15465 | 509 |
| 643 | 2300-18178WO | US03/15465 | 510 |
| 644 | 2300-18178WO | US03/15465 | 511 |
| 645 | 2300-18178WO | US03/15465 | 512 |
| 646 | 2300-18178WO | US03/15465 | 513 |
| 647 | 2300-18178WO | US03/15465 | 514 |
| 648 | 2300-18178WO | US03/15465 | 515 |
| 649 | 2300-18178WO | US03/15465 | 516 |
| 650 | 2300-18178WO | US03/15465 | 517 |
| 651 | 2300-18178WO | US03/15465 | 518 |
| 652 | 2300-18178WO | US03/15465 | 519 |
| 653 | 2300-18178WO | US03/15465 | 520 |
| 654 | 2300-18178WO | US03/15465 | 521 |
| 655 | 2300-18178WO | US03/15465 | 522 |
| 656 | 2300-18178WO | US03/15465 | 523 |
| 657 | 2300-18178WO | US03/15465 | 524 |
| 658 | 2300-18178WO | US03/15465 | 525 |
| 659 | 2300-18178WO | US03/15465 | 526 |
| 660 | 2300-18178WO | US03/15465 | 527 |
| 661 | 2300-18178WO | US03/15465 | 528 |
| 662 | 2300-18178WO | US03/15465 | 529 |
| 663 | 2300-18178WO | US03/15465 | 530 |
| 664 | 2300-18178WO | US03/15465 | 531 |
| 665 | 2300-18178WO | US03/15465 | 532 |
| 666 | 2300-18178WO | US03/15465 | 533 |
| 667 | 2300-18178WO | US03/15465 | 534 |
| 668 | 2300-18178WO | US03/15465 | 535 |
| 669 | 2300-18178WO | US03/15465 | 536 |
| 670 | 2300-18178WO | US03/15465 | 537 |
| 671 | 2300-18178WO | US03/15465 | 538 |
| 672 | 2300-18178WO | US03/15465 | 539 |
| 673 | 2300-18178WO | US03/15465 | 540 |
| 674 | 2300-18178WO | US03/15465 | 541 |
| 675 | 2300-18178WO | US03/15465 | 542 |
| 676 | 2300-18178WO | US03/15465 | 543 |
| 677 | 2300-18178WO | US03/15465 | 544 |
| 678 | 2300-18178WO | US03/15465 | 545 |
| 679 | 2300-18178WO | US03/15465 | 546 |
| 680 | 2300-18178WO | US03/15465 | 547 |
| 681 | 2300-18178WO | US03/15465 | 548 |
| 682 | 2300-18178WO | US03/15465 | 549 |
| 683 | 2300-18178WO | US03/15465 | 550 |
| 684 | 2300-18178WO | US03/15465 | 551 |
| 685 | 2300-18178WO | US03/15465 | 552 |
| 686 | 2300-18178WO | US03/15465 | 553 |
| 687 | 2300-18178WO | US03/15465 | 554 |
| 688 | 2300-18178WO | US03/15465 | 555 |
| 689 | 2300-18178WO | US03/15465 | 556 |
| 690 | 2300-18178WO | US03/15465 | 557 |
| 691 | 2300-18178WO | US03/15465 | 558 |
| 692 | 2300-18178WO | US03/15465 | 559 |
| 693 | 2300-18178WO | US03/15465 | 560 |
| 694 | 2300-18178WO | US03/15465 | 561 |
| 695 | 2300-18178WO | US03/15465 | 562 |
| 696 | 2300-18178WO | US03/15465 | 563 |
| 697 | 2300-18178WO | US03/15465 | 564 |
| 698 | 2300-18178WO | US03/15465 | 565 |
| 699 | 2300-18178WO | US03/15465 | 566 |
| 700 | 2300-18178WO | US03/15465 | 567 |
| 701 | 2300-18178WO | US03/15465 | 568 |
| 702 | 2300-18178WO | US03/15465 | 569 |
| 703 | 2300-18178WO | US03/15465 | 570 |
| 704 | 2300-18178WO | US03/15465 | 571 |
| 705 | 2300-18178WO | US03/15465 | 572 |
| 706 | 2300-18178WO | US03/15465 | 573 |
| 707 | 2300-18178WO | US03/15465 | 574 |
| 708 | 2300-18178WO | US03/15465 | 575 |
| 709 | 2300-18178WO | US03/15465 | 576 |
| 710 | 2300-18178WO | US03/15465 | 577 |
| 711 | 2300-18178WO | US03/15465 | 578 |
| 712 | 2300-18178WO | US03/15465 | 579 |
| 713 | 2300-18178WO | US03/15465 | 580 |
| 714 | 2300-18178WO | US03/15465 | 581 |
| 715 | 2300-18178WO | US03/15465 | 582 |
| 716 | 2300-18178WO | US03/15465 | 583 |
| 717 | 2300-18178WO | US03/15465 | 584 |
| 718 | 2300-18178WO | US03/15465 | 585 |
| 719 | 2300-18178WO | US03/15465 | 586 |
| 720 | 2300-18178WO | US03/15465 | 587 |
| 721 | 2300-18178WO | US03/15465 | 588 |
| 722 | 2300-18178WO | US03/15465 | 589 |
| 723 | 2300-18178WO | US03/15465 | 590 |
| 724 | 2300-18178WO | US03/15465 | 591 |
| 725 | 2300-18178WO | US03/15465 | 592 |
| 726 | 2300-18178WO | US03/15465 | 593 |
| 727 | 2300-18178WO | US03/15465 | 594 |
| 728 | 2300-18178WO | US03/15465 | 595 |
| 729 | 2300-18178WO | US03/15465 | 596 |
| 730 | 2300-18178WO | US03/15465 | 597 |
| 731 | 2300-18178WO | US03/15465 | 598 |
| 732 | 2300-18178WO | US03/15465 | 599 |
| 733 | 2300-18178WO | US03/15465 | 600 |
| 734 | 2300-18178WO | US03/15465 | 601 |
| 735 | 2300-18178WO | US03/15465 | 602 |
| 736 | 2300-18178WO | US03/15465 | 603 |
| 737 | 2300-18178WO | US03/15465 | 604 |
| 738 | 2300-18178WO | US03/15465 | 605 |
| 739 | 2300-18178WO | US03/15465 | 606 |
| 740 | 2300-18178WO | US03/15465 | 607 |
| 741 | 2300-18178WO | US03/15465 | 608 |
| 742 | 2300-18178WO | US03/15465 | 609 |
| 743 | 2300-18178WO | US03/15465 | 610 |
| 744 | 2300-18178WO | US03/15465 | 611 |
| 745 | 2300-18178WO | US03/15465 | 612 |
| 746 | 2300-18178WO | US03/15465 | 613 |
| 747 | 2300-18178WO | US03/15465 | 614 |
| 748 | 2300-18178WO | US03/15465 | 615 |
| 749 | 2300-18178WO | US03/15465 | 616 |
| 750 | 2300-18178WO | US03/15465 | 617 |
| 751 | 2300-18178WO | US03/15465 | 618 |
| 752 | 2300-18178WO | US03/15465 | 619 |
| 753 | 2300-18178WO | US03/15465 | 620 |
| 754 | 2300-18178WO | US03/15465 | 621 |
| 755 | 2300-18178WO | US03/15465 | 622 |
| 756 | 2300-18178WO | US03/15465 | 623 |
| 757 | 2300-18178WO | US03/15465 | 624 |
| 758 | 2300-18178WO | US03/15465 | 625 |
| 759 | 2300-18178WO | US03/15465 | 626 |
| 760 | 2300-18178WO | US03/15465 | 627 |
| 761 | 2300-18178WO | US03/15465 | 628 |
| 762 | 2300-18178WO | US03/15465 | 629 |
| 763 | 2300-18178WO | US03/15465 | 630 |
| 764 | 2300-18178WO | US03/15465 | 631 |
| 765 | 2300-18178WO | US03/15465 | 632 |
| 766 | 2300-18178WO | US03/15465 | 633 |
| 767 | 2300-18178WO | US03/15465 | 634 |
| 768 | 2300-18178WO | US03/15465 | 635 |
| 769 | 2300-18178WO | US03/15465 | 636 |
| 770 | 2300-18178WO | US03/15465 | 637 |
| 771 | 2300-18178WO | US03/15465 | 638 |
| 772 | 2300-18178WO | US03/15465 | 639 |
| 773 | 2300-18178WO | US03/15465 | 640 |
| 774 | 2300-18178WO | US03/15465 | 641 |
| 775 | 2300-18178WO | US03/15465 | 642 |
| 776 | 2300-18178WO | US03/15465 | 643 |
| 777 | 2300-18178WO | US03/15465 | 644 |
| 778 | 2300-18178WO | US03/15465 | 645 |
| 779 | 2300-18178WO | US03/15465 | 646 |
| 780 | 2300-18178WO | US03/15465 | 647 |
| 781 | 2300-18178WO | US03/15465 | 648 |
| 782 | 2300-18178WO | US03/15465 | 649 |
| 783 | 2300-18178WO | US03/15465 | 650 |
| 784 | 2300-18178WO | US03/15465 | 651 |
| 785 | 2300-18178WO | US03/15465 | 652 |
| 786 | 2300-18178WO | US03/15465 | 653 |
| 787 | 2300-18178WO | US03/15465 | 654 |
| 788 | 2300-18178WO | US03/15465 | 655 |
| 789 | 2300-18178WO | US03/15465 | 656 |
| 790 | 2300-18178WO | US03/15465 | 657 |
| 791 | 2300-18178WO | US03/15465 | 658 |
| 792 | 2300-18178WO | US03/15465 | 659 |
| 793 | 2300-18178WO | US03/15465 | 660 |
| 794 | 2300-18178WO | US03/15465 | 661 |
| 795 | 2300-18178WO | US03/15465 | 662 |
| 796 | 2300-18178WO | US03/15465 | 663 |
| 797 | 2300-18178WO | US03/15465 | 664 |
| 798 | 2300-18178WO | US03/15465 | 665 |
| 799 | 2300-18178WO | US03/15465 | 666 |
| 800 | 2300-18178WO | US03/15465 | 667 |
| 801 | 2300-18178WO | US03/15465 | 668 |
| 802 | 2300-18178WO | US03/15465 | 669 |
| 803 | 2300-18178WO | US03/15465 | 670 |
| 804 | 2300-18178WO | US03/15465 | 671 |
| 805 | 2300-18178WO | US03/15465 | 672 |
| 806 | 2300-18178WO | US03/15465 | 673 |
| 807 | 2300-18178WO | US03/15465 | 674 |
| 808 | 2300-18178WO | US03/15465 | 675 |
| 809 | 2300-18178WO | US03/15465 | 676 |
| 810 | 2300-18178WO | US03/15465 | 677 |
| 811 | 2300-18178WO | US03/15465 | 678 |
| 812 | 2300-18178WO | US03/15465 | 679 |
| 813 | 2300-18178WO | US03/15465 | 680 |
| 814 | 2300-18178WO | US03/15465 | 681 |
| 815 | 2300-18178WO | US03/15465 | 682 |
| 816 | 2300-18178WO | US03/15465 | 683 |
| 817 | 2300-18178WO | US03/15465 | 684 |
| 818 | 2300-18178WO | US03/15465 | 685 |
| 819 | 2300-18178WO | US03/15465 | 686 |
| 820 | 2300-18178WO | US03/15465 | 687 |
| 821 | 2300-18178WO | US03/15465 | 688 |
| 822 | 2300-18178WO | US03/15465 | 689 |
| 823 | 2300-18178WO | US03/15465 | 690 |
| 824 | 2300-18178WO | US03/15465 | 691 |
| 825 | 2300-18178WO | US03/15465 | 692 |
| 826 | 2300-18178WO | US03/15465 | 693 |
| 827 | 2300-18178WO | US03/15465 | 694 |
| 828 | 2300-18178WO | US03/15465 | 695 |
| 829 | 2300-18178WO | US03/15465 | 696 |
| 830 | 2300-18178WO | US03/15465 | 697 |
| 831 | 2300-18178WO | US03/15465 | 698 |
| 832 | 2300-18178WO | US03/15465 | 699 |
| 833 | 2300-18178WO | US03/15465 | 700 |
| 834 | 2300-18178WO | US03/15465 | 701 |
| 835 | 2300-18178WO | US03/15465 | 702 |
| 836 | 2300-18178WO | US03/15465 | 703 |
| 837 | 2300-18178WO | US03/15465 | 704 |
| 838 | 2300-18178WO | US03/15465 | 705 |
| 839 | 2300-18178WO | US03/15465 | 706 |
| 840 | 2300-18178WO | US03/15465 | 707 |
| 841 | 2300-18178WO | US03/15465 | 708 |
| 842 | 2300-18178WO | US03/15465 | 709 |
| 843 | 2300-18178WO | US03/15465 | 710 |
| 844 | 2300-18178WO | US03/15465 | 711 |
| 845 | 2300-18178WO | US03/15465 | 712 |
| 846 | 2300-18178WO | US03/15465 | 713 |
| 847 | 2300-18178WO | US03/15465 | 714 |
| 848 | 2300-18178WO | US03/15465 | 715 |
| 849 | 2300-18178WO | US03/15465 | 716 |
| 850 | 2300-18178WO | US03/15465 | 717 |
| 851 | 2300-18178WO | US03/15465 | 718 |
| 852 | 2300-18178WO | US03/15465 | 719 |
| 853 | 2300-18178WO | US03/15465 | 720 |
| 854 | 2300-18178WO | US03/15465 | 721 |
| 855 | 2300-18178WO | US03/15465 | 722 |
| 856 | 2300-18178WO | US03/15465 | 723 |
| 857 | 2300-18178WO | US03/15465 | 724 |
| 858 | 2300-18178WO | US03/15465 | 725 |
| 859 | 2300-18178WO | US03/15465 | 726 |
| 860 | 2300-18178WO | US03/15465 | 727 |
| 861 | 2300-18178WO | US03/15465 | 728 |
| 862 | 2300-18178WO | US03/15465 | 729 |
| 863 | 2300-18178WO | US03/15465 | 730 |
| 864 | 2300-18178WO | US03/15465 | 731 |
| 865 | 2300-18178WO | US03/15465 | 732 |
| 866 | 2300-18178WO | US03/15465 | 733 |
| 867 | 2300-18178WO | US03/15465 | 734 |
| 868 | 2300-18178WO | US03/15465 | 735 |
| 869 | 2300-18178WO | US03/15465 | 736 |
| 870 | 2300-18178WO | US03/15465 | 737 |
| 871 | 2300-18178WO | US03/15465 | 738 |
| 872 | 2300-18178WO | US03/15465 | 739 |
| 873 | 2300-18178WO | US03/15465 | 740 |
| 874 | 2300-18178WO | US03/15465 | 741 |
| 875 | 2300-18178WO | US03/15465 | 742 |
| 876 | 2300-18178WO | US03/15465 | 743 |
| 877 | 2300-18178WO | US03/15465 | 744 |
| 878 | 2300-18178WO | US03/15465 | 745 |
| 879 | 2300-18178WO | US03/15465 | 746 |
| 880 | 2300-18178WO | US03/15465 | 747 |
| 881 | 2300-18178WO | US03/15465 | 748 |
| 882 | 2300-18178WO | US03/15465 | 749 |
| 883 | 2300-18178WO | US03/15465 | 750 |
| 884 | 2300-18178WO | US03/15465 | 751 |
| 885 | 2300-18178WO | US03/15465 | 752 |
| 886 | 2300-18178WO | US03/15465 | 753 |
| 887 | 2300-18178WO | US03/15465 | 754 |
| 888 | 2300-18178WO | US03/15465 | 755 |
| 889 | 2300-18178WO | US03/15465 | 756 |
| 890 | 2300-18178WO | US03/15465 | 757 |
| 891 | 2300-18178WO | US03/15465 | 758 |
| 892 | 2300-18178WO | US03/15465 | 759 |
| 893 | 2300-18178WO | US03/15465 | 760 |
| 894 | 2300-18178WO | US03/15465 | 761 |
| 895 | 2300-18178WO | US03/15465 | 762 |
| 896 | 2300-18178WO | US03/15465 | 763 |
| 897 | 2300-18178WO | US03/15465 | 764 |
| 898 | 2300-18178WO | US03/15465 | 765 |
| 899 | 2300-18178WO | US03/15465 | 766 |
| 900 | 2300-18178WO | US03/15465 | 767 |
| 901 | 2300-18178WO | US03/15465 | 768 |
| 902 | 2300-18178WO | US03/15465 | 769 |
| 903 | 2300-18178WO | US03/15465 | 770 |
| 904 | 2300-18178WO | US03/15465 | 771 |
| 905 | 2300-18178WO | US03/15465 | 772 |
| 906 | 2300-18178WO | US03/15465 | 773 |
| 907 | 2300-18178WO | US03/15465 | 774 |
| 908 | 2300-18178WO | US03/15465 | 775 |
| 909 | 2300-18178WO | US03/15465 | 776 |
| 910 | 2300-18178WO | US03/15465 | 777 |
| 911 | 2300-18178WO | US03/15465 | 778 |
| 912 | 2300-18178WO | US03/15465 | 779 |
| 913 | 2300-18178WO | US03/15465 | 780 |
| 914 | 2300-18178WO | US03/15465 | 781 |
| 915 | 2300-18178WO | US03/15465 | 782 |
| 916 | 2300-18178WO | US03/15465 | 783 |
| 917 | 2300-18178WO | US03/15465 | 784 |
| 918 | 2300-18178WO | US03/15465 | 785 |
| 919 | 2300-18178WO | US03/15465 | 786 |
| 920 | 2300-18178WO | US03/15465 | 787 |
| 921 | 2300-18178WO | US03/15465 | 788 |
| 922 | 2300-18178WO | US03/15465 | 789 |
| 923 | 2300-18178WO | US03/15465 | 790 |
| 924 | 2300-18178WO | US03/15465 | 791 |
| 925 | 2300-18178WO | US03/15465 | 792 |
| 926 | 2300-18178WO | US03/15465 | 793 |
| 927 | 2300-18178WO | US03/15465 | 794 |
| 928 | 2300-18178WO | US03/15465 | 795 |
| 929 | 2300-18178WO | US03/15465 | 796 |
| 930 | 2300-18178WO | US03/15465 | 797 |
| 931 | 2300-18178WO | US03/15465 | 798 |
| 932 | 2300-18178WO | US03/15465 | 799 |
| 933 | 2300-18178WO | US03/15465 | 800 |
| 934 | 2300-18178WO | US03/15465 | 801 |
| 935 | 2300-18178WO | US03/15465 | 802 |
| 936 | 2300-18178WO | US03/15465 | 803 |
| 937 | 2300-18178WO | US03/15465 | 804 |
| 938 | 2300-18178WO | US03/15465 | 805 |
| 939 | 2300-18178WO | US03/15465 | 806 |
| 940 | 2300-18178WO | US03/15465 | 807 |
| 941 | 2300-18178WO | US03/15465 | 808 |
| 942 | 2300-18178WO | US03/15465 | 809 |
| 943 | 2300-18178WO | US03/15465 | 810 |
| 944 | 2300-18178WO | US03/15465 | 811 |
| 945 | 2300-18178WO | US03/15465 | 812 |
| 946 | 2300-18178WO | US03/15465 | 813 |
| 947 | 2300-18178WO | US03/15465 | 814 |
| 948 | 2300-18178WO | US03/15465 | 815 |
| 949 | 2300-18178WO | US03/15465 | 816 |
| 950 | 2300-18178WO | US03/15465 | 817 |
| 951 | 2300-18178WO | US03/15465 | 818 |
| 952 | 2300-18178WO | US03/15465 | 819 |
| 953 | 2300-18178WO | US03/15465 | 820 |
| 954 | 2300-18178WO | US03/15465 | 821 |
| 955 | 2300-18178WO | US03/15465 | 822 |
| 956 | 2300-18178WO | US03/15465 | 823 |
| 957 | 2300-18178WO | US03/15465 | 824 |
| 958 | 2300-18178WO | US03/15465 | 825 |
| 959 | 2300-18178WO | US03/15465 | 826 |
| 960 | 2300-18178WO | US03/15465 | 827 |
| 961 | 2300-18178WO | US03/15465 | 828 |
| 962 | 2300-18178WO | US03/15465 | 829 |
| 963 | 2300-18178WO | US03/15465 | 830 |
| 964 | 2300-18178WO | US03/15465 | 831 |
| 965 | 2300-18178WO | US03/15465 | 832 |
| 966 | 2300-18178WO | US03/15465 | 833 |
| 967 | 2300-18178WO | US03/15465 | 834 |
| 968 | 2300-18178WO | US03/15465 | 835 |
| 969 | 2300-18178WO | US03/15465 | 836 |
| 970 | 2300-18178WO | US03/15465 | 837 |
| 971 | 2300-18178WO | US03/15465 | 838 |
| 972 | 2300-18178WO | US03/15465 | 839 |
| 973 | 2300-18178WO | US03/15465 | 840 |
| 974 | 2300-18178WO | US03/15465 | 841 |
| 975 | 2300-18178WO | US03/15465 | 842 |
| 976 | 2300-18178WO | US03/15465 | 843 |
| 977 | 2300-18178WO | US03/15465 | 844 |
| 978 | 2300-18178WO | US03/15465 | 845 |
| 979 | 2300-18178WO | US03/15465 | 846 |
| 980 | 2300-18178WO | US03/15465 | 847 |
| 981 | 2300-18178WO | US03/15465 | 848 |
| 982 | 2300-18178WO | US03/15465 | 849 |
| 983 | 2300-18178WO | US03/15465 | 850 |
| 984 | 2300-18178WO | US03/15465 | 851 |
| 985 | 2300-18178WO | US03/15465 | 852 |
| 986 | 2300-18178WO | US03/15465 | 853 |
| 987 | 2300-18178WO | US03/15465 | 854 |
| 988 | 2300-18178WO | US03/15465 | 855 |
| 989 | 2300-18178WO | US03/15465 | 856 |
| 990 | 2300-18178WO | US03/15465 | 857 |
| 991 | 2300-18178WO | US03/15465 | 858 |
| 992 | 2300-18178WO | US03/15465 | 859 |
| 993 | 2300-18178WO | US03/15465 | 860 |
| 994 | 2300-18178WO | US03/15465 | 861 |
| 995 | 2300-18178WO | US03/15465 | 862 |
| 996 | 2300-18178WO | US03/15465 | 863 |
| 997 | 2300-18178WO | US03/15465 | 864 |
| 998 | 2300-18178WO | US03/15465 | 865 |
| 999 | 2300-18178WO | US03/15465 | 866 |
| 1000 | 2300-18178WO | US03/15465 | 867 |
| 1001 | 2300-18178WO | US03/15465 | 868 |
| 1002 | 2300-18178WO | US03/15465 | 869 |
| 1003 | 2300-18178WO | US03/15465 | 870 |
| 1004 | 2300-18178WO | US03/15465 | 871 |
| 1005 | 2300-18178WO | US03/15465 | 872 |
| 1006 | 2300-18178WO | US03/15465 | 873 |
| 1007 | 2300-18178WO | US03/15465 | 874 |
| 1008 | 2300-18178WO | US03/15465 | 875 |
| 1009 | 2300-18178WO | US03/15465 | 876 |
| 1010 | 2300-18178WO | US03/15465 | 877 |
| 1011 | 2300-18178WO | US03/15465 | 878 |
| 1012 | 2300-18178WO | US03/15465 | 879 |
| 1013 | 2300-18178WO | US03/15465 | 880 |
| 1014 | 2300-18178WO | US03/15465 | 881 |
| 1015 | 2300-18178WO | US03/15465 | 882 |
| 1016 | 2300-18178WO | US03/15465 | 883 |
| 1017 | 2300-18178WO | US03/15465 | 884 |
| 1018 | 2300-18178WO | US03/15465 | 885 |
| 1019 | 2300-18178WO | US03/15465 | 886 |
| 1020 | 2300-18178WO | US03/15465 | 887 |
| 1021 | 2300-18178WO | US03/15465 | 888 |
| 1022 | 2300-18178WO | US03/15465 | 889 |
| 1023 | 2300-18178WO | US03/15465 | 890 |
| 1024 | 2300-18178WO | US03/15465 | 891 |
| 1025 | 2300-18178WO | US03/15465 | 892 |
| 1026 | 2300-18178WO | US03/15465 | 893 |
| 1027 | 2300-18178WO | US03/15465 | 894 |
| 1028 | 2300-18178WO | US03/15465 | 895 |
| 1029 | 2300-18178WO | US03/15465 | 896 |
| 1030 | 2300-18178WO | US03/15465 | 897 |
| 1031 | 2300-18178WO | US03/15465 | 898 |
| 1032 | 2300-18178WO | US03/15465 | 899 |
| 1033 | 2300-18178WO | US03/15465 | 900 |
| 1034 | 2300-18178WO | US03/15465 | 901 |
| 1035 | 2300-18178WO | US03/15465 | 902 |
| 1036 | 2300-18178WO | US03/15465 | 903 |
| 1037 | 2300-18178WO | US03/15465 | 904 |
| 1038 | 2300-18178WO | US03/15465 | 905 |
| 1039 | 2300-18178WO | US03/15465 | 906 |
| 1040 | 2300-18178WO | US03/15465 | 907 |
| 1041 | 2300-18178WO | US03/15465 | 908 |
| 1042 | 2300-18178WO | US03/15465 | 909 |
| 1043 | 2300-18178WO | US03/15465 | 910 |
| 1044 | 2300-18178WO | US03/15465 | 911 |
| 1045 | 2300-18178WO | US03/15465 | 912 |
| 1046 | 2300-18178WO | US03/15465 | 913 |
| 1047 | 2300-18178WO | US03/15465 | 914 |
| 1048 | 2300-18178WO | US03/15465 | 915 |
| 1049 | 2300-18178WO | US03/15465 | 916 |
| 1050 | 2300-18178WO | US03/15465 | 917 |
| 1051 | 2300-18178WO | US03/15465 | 918 |
| 1052 | 2300-18178WO | US03/15465 | 919 |
| 1053 | 2300-18178WO | US03/15465 | 920 |
| 1054 | 2300-18178WO | US03/15465 | 921 |
| 1055 | 2300-18178WO | US03/15465 | 922 |
| 1056 | 2300-18178WO | US03/15465 | 923 |
| 1057 | 2300-18178WO | US03/15465 | 924 |
| 1058 | 2300-18178WO | US03/15465 | 925 |
| 1059 | 2300-18178WO | US03/15465 | 926 |
| 1060 | 2300-18178WO | US03/15465 | 927 |
| 1061 | 2300-18178WO | US03/15465 | 928 |
| 1062 | 2300-18178WO | US03/15465 | 929 |
| 1063 | 2300-18178WO | US03/15465 | 930 |
| 1064 | 2300-18178WO | US03/15465 | 931 |
| 1065 | 2300-18178WO | US03/15465 | 932 |
| 1066 | 2300-18178WO | US03/15465 | 933 |
| 1067 | 2300-18178WO | US03/15465 | 934 |
| 1068 | 2300-18178WO | US03/15465 | 935 |
| 1069 | 2300-18178WO | US03/15465 | 936 |
| 1070 | 2300-18178WO | US03/15465 | 937 |
| 1071 | 2300-18178WO | US03/15465 | 938 |
| 1072 | 2300-18178WO | US03/15465 | 939 |
| 1073 | 2300-18178WO | US03/15465 | 940 |
| 1074 | 2300-18178WO | US03/15465 | 941 |
| 1075 | 2300-18178WO | US03/15465 | 942 |
| 1076 | 2300-18178WO | US03/15465 | 943 |
| 1077 | 2300-18178WO | US03/15465 | 944 |
| 1078 | 2300-18178WO | US03/15465 | 945 |
| 1079 | 2300-18178WO | US03/15465 | 946 |
| 1080 | 2300-18178WO | US03/15465 | 947 |
| 1081 | 2300-18178WO | US03/15465 | 948 |
| 1082 | 2300-18178WO | US03/15465 | 949 |
| 1083 | 2300-18178WO | US03/15465 | 950 |
| 1084 | 2300-18178WO | US03/15465 | 951 |
| 1085 | 2300-18178WO | US03/15465 | 952 |
| 1086 | 2300-18178WO | US03/15465 | 953 |
| 1087 | 2300-18178WO | US03/15465 | 954 |
| 1088 | 2300-18178WO | US03/15465 | 955 |
| 1089 | 2300-18178WO | US03/15465 | 956 |
| 1090 | 2300-18178WO | US03/15465 | 957 |
| 1091 | 2300-18178WO | US03/15465 | 958 |
| 1092 | 2300-18178WO | US03/15465 | 959 |
| 1093 | 2300-18178WO | US03/15465 | 960 |
| 1094 | 2300-18178WO | US03/15465 | 961 |
| 1095 | 2300-18178WO | US03/15465 | 962 |
| 1096 | 2300-18178WO | US03/15465 | 963 |
| 1097 | 2300-18178WO | US03/15465 | 964 |
| 1098 | 2300-18178WO | US03/15465 | 965 |
| 1099 | 2300-18178WO | US03/15465 | 966 |
| 1100 | 2300-18178WO | US03/15465 | 967 |
| 1101 | 2300-18178WO | US03/15465 | 968 |
| 1102 | 2300-18178WO | US03/15465 | 969 |
| 1103 | 2300-18178WO | US03/15465 | 970 |
| 1104 | 2300-18178WO | US03/15465 | 971 |
| 1105 | 2300-18178WO | US03/15465 | 972 |
| 1106 | 2300-18178WO | US03/15465 | 973 |
| 1107 | 2300-18178WO | US03/15465 | 974 |
| 1108 | 2300-18178WO | US03/15465 | 975 |
| 1109 | 2300-18178WO | US03/15465 | 976 |
| 1110 | 2300-18178WO | US03/15465 | 977 |
| 1111 | 2300-18178WO | US03/15465 | 978 |
| 1112 | 2300-18178WO | US03/15465 | 979 |
| 1113 | 2300-18178WO | US03/15465 | 980 |
| 1114 | 2300-18178WO | US03/15465 | 981 |
| 1115 | 2300-18178WO | US03/15465 | 982 |
| 1116 | 2300-18178WO | US03/15465 | 983 |
| 1117 | 2300-18178WO | US03/15465 | 984 |
| 1118 | 2300-18178WO | US03/15465 | 985 |
| 1119 | 2300-18178WO | US03/15465 | 986 |
| 1120 | 2300-18178WO | US03/15465 | 987 |
| 1121 | 2300-18178WO | US03/15465 | 988 |
| 1122 | 2300-18178WO | US03/15465 | 989 |
| 1123 | 2300-18178WO | US03/15465 | 990 |
| 1124 | 2300-18178WO | US03/15465 | 991 |
| 1125 | 2300-18178WO | US03/15465 | 992 |
| 1126 | 2300-18178WO | US03/15465 | 993 |
| 1127 | 2300-18178WO | US03/15465 | 994 |
| 1128 | 2300-18178WO | US03/15465 | 995 |
| 1129 | 2300-18178WO | US03/15465 | 996 |
| 1130 | 2300-18178WO | US03/15465 | 997 |
| 1131 | 2300-18178WO | US03/15465 | 998 |
| 1132 | 2300-18178WO | US03/15465 | 999 |
| 1133 | 2300-18178WO | US03/15465 | 1000 |
| 1134 | 2300-18178WO | US03/15465 | 1001 |
| 1135 | 2300-18178WO | US03/15465 | 1002 |
| 1136 | 2300-18178WO | US03/15465 | 1003 |
| 1137 | 2300-18178WO | US03/15465 | 1004 |
| 1138 | 2300-18178WO | US03/15465 | 1005 |
| 1139 | 2300-18178WO | US03/15465 | 1006 |
| 1140 | 2300-18178WO | US03/15465 | 1007 |
| 1141 | 2300-18178WO | US03/15465 | 1008 |
| 1142 | 2300-18178WO | US03/15465 | 1009 |
| 1143 | 2300-18178WO | US03/15465 | 1010 |
| 1144 | 2300-18178WO | US03/15465 | 1011 |
| 1145 | 2300-18178WO | US03/15465 | 1012 |
| 1146 | 2300-18178WO | US03/15465 | 1013 |
| 1147 | 2300-18178WO | US03/15465 | 1014 |
| 1148 | 2300-18178WO | US03/15465 | 1015 |
| 1149 | 2300-18178WO | US03/15465 | 1016 |
| 1150 | 2300-18178WO | US03/15465 | 1017 |
| 1151 | 2300-18178WO | US03/15465 | 1018 |
| 1152 | 2300-18178WO | US03/15465 | 1019 |
| 1153 | 2300-18178WO | US03/15465 | 1020 |
| 1154 | 2300-18178WO | US03/15465 | 1021 |
| 1155 | 2300-18178WO | US03/15465 | 1022 |
| 1156 | 2300-18178WO | US03/15465 | 1023 |
| 1157 | 2300-18178WO | US03/15465 | 1024 |
| 1158 | 2300-18178WO | US03/15465 | 1025 |
| 1159 | 2300-18178WO | US03/15465 | 1026 |
| 1160 | 2300-18178WO | US03/15465 | 1027 |
| 1161 | 2300-18178WO | US03/15465 | 1028 |
| 1162 | 2300-18178WO | US03/15465 | 1029 |
| 1163 | 2300-18178WO | US03/15465 | 1030 |
| 1164 | 2300-18178WO | US03/15465 | 1031 |
| 1165 | 2300-18178WO | US03/15465 | 1032 |
| 1166 | 2300-18178WO | US03/15465 | 1033 |
| 1167 | 2300-18178WO | US03/15465 | 1034 |
| 1168 | 2300-18178WO | US03/15465 | 1035 |
| 1169 | 2300-18178WO | US03/15465 | 1036 |
| 1170 | 2300-18178WO | US03/15465 | 1037 |
| 1171 | 2300-18178WO | US03/15465 | 1038 |
| 1172 | 2300-18178WO | US03/15465 | 1039 |
| 1173 | 2300-18178WO | US03/15465 | 1040 |
| 1174 | 2300-18178WO | US03/15465 | 1041 |
| 1175 | 2300-18178WO | US03/15465 | 1042 |
| 1176 | 2300-18178WO | US03/15465 | 1043 |
| 1177 | 2300-18178WO | US03/15465 | 1044 |
| 1178 | 2300-18178WO | US03/15465 | 1045 |
| 1179 | 2300-18178WO | US03/15465 | 1046 |
| 1180 | 2300-18178WO | US03/15465 | 1047 |
| 1181 | 2300-18178WO | US03/15465 | 1048 |
| 1182 | 2300-18178WO | US03/15465 | 1049 |
| 1183 | 2300-18178WO | US03/15465 | 1050 |
| 1184 | 2300-18178WO | US03/15465 | 1051 |
| 1185 | 2300-18178WO | US03/15465 | 1052 |
| 1186 | 2300-18178WO | US03/15465 | 1053 |
| 1187 | 2300-18178WO | US03/15465 | 1054 |
| 1188 | 2300-18178WO | US03/15465 | 1055 |
| 1189 | 2300-18178WO | US03/15465 | 1056 |
| 1190 | 2300-18178WO | US03/15465 | 1057 |
| 1191 | 2300-18178WO | US03/15465 | 1058 |
| 1192 | 2300-18178WO | US03/15465 | 1059 |
| 1193 | 2300-18178WO | US03/15465 | 1060 |
| 1194 | 2300-18178WO | US03/15465 | 1061 |
| 1195 | 2300-18178WO | US03/15465 | 1062 |
| 1196 | 2300-18178WO | US03/15465 | 1063 |
| 1197 | 2300-18178WO | US03/15465 | 1064 |
| 1198 | 2300-18178WO | US03/15465 | 1065 |
| 1199 | 2300-18178WO | US03/15465 | 1066 |
| 1200 | 2300-18178WO | US03/15465 | 1067 |
| 1201 | 2300-18178WO | US03/15465 | 1068 |
| 1202 | 2300-18178WO | US03/15465 | 1069 |
| 1203 | 2300-18178WO | US03/15465 | 1070 |
| 1204 | 2300-18178WO | US03/15465 | 1071 |
| 1205 | 2300-18178WO | US03/15465 | 1072 |
| 1206 | 2300-18178WO | US03/15465 | 1073 |
| 1207 | 2300-18178WO | US03/15465 | 1074 |
| 1208 | 2300-18178WO | US03/15465 | 1075 |
| 1209 | 2300-18178WO | US03/15465 | 1076 |
| 1210 | 2300-18178WO | US03/15465 | 1077 |
| 1211 | 2300-18178WO | US03/15465 | 1078 |
| 1212 | 2300-18178WO | US03/15465 | 1079 |
| 1213 | 2300-18178WO | US03/15465 | 1080 |
| 1214 | 2300-18178WO | US03/15465 | 1081 |
| 1215 | 2300-18178WO | US03/15465 | 1082 |
| 1216 | 2300-18178WO | US03/15465 | 1083 |
| 1217 | 2300-18178WO | US03/15465 | 1084 |
| 1218 | 2300-18178WO | US03/15465 | 1085 |
| 1219 | 2300-18178WO | US03/15465 | 1086 |
| 1220 | 2300-18178WO | US03/15465 | 1087 |
| 1221 | 2300-18178WO | US03/15465 | 1088 |
| 1222 | 2300-18178WO | US03/15465 | 1089 |
| 1223 | 2300-18178WO | US03/15465 | 1090 |
| 1224 | 2300-18178WO | US03/15465 | 1091 |
| 1225 | 2300-18178WO | US03/15465 | 1092 |
| 1226 | 2300-18178WO | US03/15465 | 1093 |
| 1227 | 2300-18178WO | US03/15465 | 1094 |
| 1228 | 2300-18178WO | US03/15465 | 1095 |
| 1229 | 2300-18178WO | US03/15465 | 1096 |
| 1230 | 2300-18178WO | US03/15465 | 1097 |
| 1231 | 2300-18178WO | US03/15465 | 1098 |
| 1232 | 2300-18178WO | US03/15465 | 1099 |
| 1233 | 2300-18178WO | US03/15465 | 1100 |
| 1234 | 2300-18178WO | US03/15465 | 1101 |
| 1235 | 2300-18178WO | US03/15465 | 1102 |
| 1236 | 2300-18178WO | US03/15465 | 1103 |
| 1237 | 2300-18178WO | US03/15465 | 1104 |
| 1238 | 2300-18178WO | US03/15465 | 1105 |
| 1239 | 2300-18178WO | US03/15465 | 1106 |
| 1240 | 2300-18178WO | US03/15465 | 1107 |
| 1241 | 2300-18178WO | US03/15465 | 1108 |
| 1242 | 2300-18178WO | US03/15465 | 1109 |
| 1243 | 2300-18178WO | US03/15465 | 1110 |
| 1244 | 2300-18178WO | US03/15465 | 1111 |
| 1245 | 2300-18178WO | US03/15465 | 1112 |
| 1246 | 2300-18178WO | US03/15465 | 1113 |
| 1247 | 2300-18178WO | US03/15465 | 1114 |
| 1248 | 2300-18178WO | US03/15465 | 1115 |
| 1249 | 2300-18178WO | US03/15465 | 1116 |
| 1250 | 2300-18178WO | US03/15465 | 1117 |
| 1251 | 2300-18178WO | US03/15465 | 1118 |
| 1252 | 2300-18178WO | US03/15465 | 1119 |
| 1253 | 2300-18178WO | US03/15465 | 1120 |
| 1254 | 2300-18178WO | US03/15465 | 1121 |
| 1255 | 2300-18178WO | US03/15465 | 1122 |
| 1256 | 2300-18178WO | US03/15465 | 1123 |
| 1257 | 2300-18178WO | US03/15465 | 1124 |
| 1258 | 2300-18178WO | US03/15465 | 1125 |
| 1259 | 2300-18178WO | US03/15465 | 1126 |
| 1260 | 2300-18178WO | US03/15465 | 1127 |
| 1261 | 2300-18178WO | US03/15465 | 1128 |
| 1262 | 2300-18178WO | US03/15465 | 1129 |
| 1263 | 2300-18178WO | US03/15465 | 1130 |
| 1264 | 2300-18178WO | US03/15465 | 1131 |
| 1265 | 2300-18178WO | US03/15465 | 1132 |
| 1266 | 2300-18178WO | US03/15465 | 1133 |
| 1267 | 2300-18178WO | US03/15465 | 1134 |
| 1268 | 2300-18178WO | US03/15465 | 1135 |
| 1269 | 2300-18178WO | US03/15465 | 1136 |
| 1270 | 2300-18178WO | US03/15465 | 1137 |
| 1271 | 2300-18178WO | US03/15465 | 1138 |
| 1272 | 2300-18178WO | US03/15465 | 1139 |
| 1273 | 2300-18178WO | US03/15465 | 1140 |
| 1274 | 2300-18178WO | US03/15465 | 1141 |
| 1275 | 2300-18178WO | US03/15465 | 1142 |
| 1276 | 2300-18178WO | US03/15465 | 1143 |
| 1277 | 2300-18178WO | US03/15465 | 1144 |
| 1278 | 2300-18178WO | US03/15465 | 1145 |
| 1279 | 2300-18178WO | US03/15465 | 1146 |
| 1280 | 2300-18178WO | US03/15465 | 1147 |
| 1281 | 2300-18178WO | US03/15465 | 1148 |
| 1282 | 2300-18178WO | US03/15465 | 1149 |
| 1283 | 2300-18178WO | US03/15465 | 1150 |
| 1284 | 2300-18178WO | US03/15465 | 1151 |
| 1285 | 2300-18178WO | US03/15465 | 1152 |
| 1286 | 2300-18178WO | US03/15465 | 1153 |
| 1287 | 2300-18178WO | US03/15465 | 1154 |
| 1288 | 2300-18178WO | US03/15465 | 1155 |
| 1289 | 2300-18178WO | US03/15465 | 1156 |
| 1290 | 2300-18178WO | US03/15465 | 1157 |
| 1291 | 2300-18178WO | US03/15465 | 1158 |
| 1292 | 2300-18178WO | US03/15465 | 1159 |
| 1293 | 2300-18178WO | US03/15465 | 1160 |
| 1294 | 2300-18178WO | US03/15465 | 1161 |
| 1295 | 2300-18178WO | US03/15465 | 1162 |
| 1296 | 2300-18178WO | US03/15465 | 1163 |
| 1297 | 2300-18178WO | US03/15465 | 1164 |
| 1298 | 2300-18178WO | US03/15465 | 1165 |
| 1299 | 2300-18178WO | US03/15465 | 1166 |
| 1300 | 2300-18178WO | US03/15465 | 1167 |
| 1301 | 2300-18178WO | US03/15465 | 1168 |
| 1302 | 2300-18178WO | US03/15465 | 1169 |
| 1303 | 2300-18178WO | US03/15465 | 1170 |
| 1304 | 2300-18178WO | US03/15465 | 1171 |
| 1305 | 2300-18178WO | US03/15465 | 1172 |
| 1306 | 2300-18178WO | US03/15465 | 1173 |
| 1307 | 2300-18178WO | US03/15465 | 1174 |
| 1308 | 2300-18178WO | US03/15465 | 1175 |
| 1309 | 2300-18178WO | US03/15465 | 1176 |
| 1310 | 2300-18178WO | US03/15465 | 1177 |
| 1311 | 2300-18178WO | US03/15465 | 1178 |
| 1312 | 2300-18178WO | US03/15465 | 1179 |
| 1313 | 2300-18178WO | US03/15465 | 1180 |
| 1314 | 2300-18178WO | US03/15465 | 1181 |
| 1315 | 2300-18178WO | US03/15465 | 1182 |
| 1316 | 2300-18178WO | US03/15465 | 1183 |
| 1317 | 2300-18178WO | US03/15465 | 1184 |
| 1318 | 2300-18178WO | US03/15465 | 1185 |
| 1319 | 2300-18178WO | US03/15465 | 1186 |
| 1320 | 2300-18178WO | US03/15465 | 1187 |
| 1321 | 2300-18178WO | US03/15465 | 1188 |
| 1322 | 2300-18178WO | US03/15465 | 1189 |
| 1323 | 2300-18178WO | US03/15465 | 1190 |
| 1324 | 2300-18178WO | US03/15465 | 1191 |
| 1325 | 2300-18178WO | US03/15465 | 1192 |
| 1326 | 2300-18178WO | US03/15465 | 1193 |
| 1327 | 2300-18178WO | US03/15465 | 1194 |
| 1328 | 2300-18178WO | US03/15465 | 1195 |
| 1329 | 2300-18178WO | US03/15465 | 1196 |
| 1330 | 2300-18178WO | US03/15465 | 1197 |
| 1331 | 2300-18178WO | US03/15465 | 1198 |
| 1332 | 2300-18178WO | US03/15465 | 1199 |
| 1333 | 2300-18178WO | US03/15465 | 1200 |
| 1334 | 2300-18178WO | US03/15465 | 1201 |
| 1335 | 2300-18178WO | US03/15465 | 1202 |
| 1336 | 2300-18178WO | US03/15465 | 1203 |
| 1337 | 2300-18178WO | US03/15465 | 1204 |
| 1338 | 2300-18178WO | US03/15465 | 1205 |
| 1339 | 2300-18178WO | US03/15465 | 1206 |
| 1340 | 2300-18178WO | US03/15465 | 1207 |
| 1341 | 2300-18178WO | US03/15465 | 1208 |
| 1342 | 2300-18178WO | US03/15465 | 1209 |
| 1343 | 2300-18178WO | US03/15465 | 1210 |
| 1344 | 2300-18178WO | US03/15465 | 1211 |
| 1345 | 2300-18178WO | US03/15465 | 1212 |
| 1346 | 2300-18178WO | US03/15465 | 1213 |
| 1347 | 2300-18178WO | US03/15465 | 1214 |
| 1348 | 2300-18178WO | US03/15465 | 1215 |
| 1349 | 2300-18178WO | US03/15465 | 1216 |
| 1350 | 2300-18178WO | US03/15465 | 1217 |
| 1351 | 2300-18178WO | US03/15465 | 1218 |
| 1352 | 2300-18178WO | US03/15465 | 1219 |
| 1353 | 2300-18178WO | US03/15465 | 1220 |
| 1354 | 2300-18178WO | US03/15465 | 1221 |
| 1355 | 2300-18178WO | US03/15465 | 1222 |
| 1356 | 2300-18178WO | US03/15465 | 1223 |
| 1357 | 2300-18178WO | US03/15465 | 1224 |
| 1358 | 2300-18178WO | US03/15465 | 1225 |
| 1359 | 2300-18178WO | US03/15465 | 1226 |
| 1360 | 2300-18178WO | US03/15465 | 1227 |
| 1361 | 2300-18178WO | US03/15465 | 1228 |
| 1362 | 2300-18178WO | US03/15465 | 1229 |
| 1363 | 2300-18178WO | US03/15465 | 1230 |
| 1364 | 2300-18178WO | US03/15465 | 1231 |
| 1365 | 2300-18178WO | US03/15465 | 1232 |
| 1366 | 2300-18178WO | US03/15465 | 1233 |
| 1367 | 2300-18178WO | US03/15465 | 1234 |
| 1368 | 2300-18178WO | US03/15465 | 1235 |
| 1369 | 2300-18178WO | US03/15465 | 1236 |
| 1370 | 2300-18178WO | US03/15465 | 1237 |
| 1371 | 2300-18178WO | US03/15465 | 1238 |
| 1372 | 2300-18178WO | US03/15465 | 1239 |
| 1373 | 2300-18178WO | US03/15465 | 1240 |
| 1374 | 2300-18178WO | US03/15465 | 1241 |
| 1375 | 2300-18178WO | US03/15465 | 1242 |
| 1376 | 2300-18178WO | US03/15465 | 1243 |
| 1377 | 2300-18178WO | US03/15465 | 1244 |
| 1378 | 2300-18178WO | US03/15465 | 1245 |
| 1379 | 2300-18178WO | US03/15465 | 1246 |
| 1380 | 2300-18178WO | US03/15465 | 1247 |
| 1381 | 2300-18178WO | US03/15465 | 1248 |
| 1382 | 2300-18178WO | US03/15465 | 1249 |
| 1383 | 2300-18178WO | US03/15465 | 1250 |
| 1384 | 2300-18178WO | US03/15465 | 1251 |
| 1385 | 2300-18178WO | US03/15465 | 1252 |
| 1386 | 2300-18178WO | US03/15465 | 1253 |
| 1387 | 2300-18178WO | US03/15465 | 1254 |
| 1388 | 2300-18178WO | US03/15465 | 1255 |
| 1389 | 2300-18178WO | US03/15465 | 1256 |
| 1390 | 2300-18178WO | US03/15465 | 1257 |
| 1391 | 2300-18178WO | US03/15465 | 1258 |
| 1392 | 2300-18178WO | US03/15465 | 1259 |
| 1393 | 2300-18178WO | US03/15465 | 1260 |
| 1394 | 2300-18178WO | US03/15465 | 1261 |
| 1395 | 2300-18178WO | US03/15465 | 1262 |
| 1396 | 2300-18178WO | US03/15465 | 1263 |
| 1397 | 2300-18178WO | US03/15465 | 1264 |
| 1398 | 2300-18178WO | US03/15465 | 1265 |
| 1399 | 2300-18178WO | US03/15465 | 1266 |
| 1400 | 2300-18178WO | US03/15465 | 1267 |
| 1401 | 2300-18178WO | US03/15465 | 1268 |
| 1402 | 2300-18178WO | US03/15465 | 1269 |
| 1403 | 2300-18178WO | US03/15465 | 1270 |
| 1404 | 2300-18178WO | US03/15465 | 1271 |
| 1405 | 2300-18178WO | US03/15465 | 1272 |
| 1406 | 2300-18178WO | US03/15465 | 1273 |
| 1407 | 2300-18178WO | US03/15465 | 1274 |
| 1408 | 2300-18178WO | US03/15465 | 1275 |
| 1409 | 2300-18178WO | US03/15465 | 1276 |
| 1410 | 2300-18178WO | US03/15465 | 1277 |
| 1411 | 2300-18178WO | US03/15465 | 1278 |
| 1412 | 2300-18178WO | US03/15465 | 1279 |
| 1413 | 2300-18178WO | US03/15465 | 1280 |
| 1414 | 2300-18178WO | US03/15465 | 1281 |
| 1415 | 2300-18178WO | US03/15465 | 1282 |
| 1416 | 2300-18178WO | US03/15465 | 1283 |
| 1417 | 2300-18178WO | US03/15465 | 1284 |
| 1418 | 2300-18178WO | US03/15465 | 1285 |
| 1419 | 2300-18178WO | US03/15465 | 1286 |
| 1420 | 2300-18178WO | US03/15465 | 1287 |
| 1421 | 2300-18178WO | US03/15465 | 1288 |
| 1422 | 2300-18178WO | US03/15465 | 1289 |
| 1423 | 2300-18178WO | US03/15465 | 1290 |
| 1424 | 2300-18178WO | US03/15465 | 1291 |
| 1425 | 2300-18178WO | US03/15465 | 1292 |
| 1426 | 2300-18178WO | US03/15465 | 1293 |
| 1427 | 2300-18178WO | US03/15465 | 1294 |
| 1428 | 2300-18178WO | US03/15465 | 1295 |
| 1429 | 2300-18178WO | US03/15465 | 1296 |
| 1430 | 2300-18178WO | US03/15465 | 1297 |
| 1431 | 2300-18178WO | US03/15465 | 1298 |
| 1432 | 2300-18178WO | US03/15465 | 1299 |
| 1433 | 2300-18178WO | US03/15465 | 1300 |
| 1434 | 2300-18178WO | US03/15465 | 1301 |
| 1435 | 2300-18178WO | US03/15465 | 1302 |
| 1436 | 2300-18178WO | US03/15465 | 1303 |
| 1437 | 2300-18178WO | US03/15465 | 1304 |
| 1438 | 2300-18178WO | US03/15465 | 1305 |
| 1439 | 2300-18178WO | US03/15465 | 1306 |
| 1440 | 2300-18178WO | US03/15465 | 1307 |
| 1441 | 2300-18178WO | US03/15465 | 1308 |
| 1442 | 2300-18178WO | US03/15465 | 1309 |
| 1443 | 2300-18178WO | US03/15465 | 1310 |
| 1444 | 2300-18178WO | US03/15465 | 1311 |
| 1445 | 2300-18178WO | US03/15465 | 1312 |
| 1446 | 2300-18178WO | US03/15465 | 1313 |
| 1447 | 2300-18178WO | US03/15465 | 1314 |
| 1448 | 2300-18178WO | US03/15465 | 1315 |
| 1449 | 2300-18178WO | US03/15465 | 1316 |
| 1450 | 2300-18178WO | US03/15465 | 1317 |
| 1451 | 2300-18178WO | US03/15465 | 1318 |
| 1452 | 2300-18178WO | US03/15465 | 1319 |
| 1453 | 2300-18178WO | US03/15465 | 1320 |
| 1454 | 2300-18178WO | US03/15465 | 1321 |
| 1455 | 2300-18178WO | US03/15465 | 1322 |
| 1456 | 2300-18178WO | US03/15465 | 1323 |
| 1457 | 2300-18178WO | US03/15465 | 1324 |
| 1458 | 2300-18178WO | US03/15465 | 1325 |
| 1459 | 2300-18178WO | US03/15465 | 1326 |
| 1460 | 2300-18178WO | US03/15465 | 1327 |
| 1461 | 2300-18178WO | US03/15465 | 1328 |
| 1462 | 2300-18178WO | US03/15465 | 1329 |
| 1463 | 2300-18178WO | US03/15465 | 1330 |
| 1464 | 2300-18178WO | US03/15465 | 1331 |
| 1465 | 2300-18178WO | US03/15465 | 1332 |
| 1466 | 2300-18178WO | US03/15465 | 1333 |
| 1467 | 2300-18178WO | US03/15465 | 1334 |
| 1468 | 2300-18178WO | US03/15465 | 1335 |
| 1469 | 2300-18178WO | US03/15465 | 1336 |
| 1470 | 2300-18178WO | US03/15465 | 1337 |
| 1471 | 2300-18178WO | US03/15465 | 1338 |
| 1472 | 2300-18178WO | US03/15465 | 1339 |
| 1473 | 2300-18178WO | US03/15465 | 1340 |
| 1474 | 2300-18178WO | US03/15465 | 1341 |
| 1475 | 2300-18178WO | US03/15465 | 1342 |
| 1476 | 2300-18178WO | US03/15465 | 1343 |
| 1477 | 2300-18178WO | US03/15465 | 1344 |
| 1478 | 2300-18178WO | US03/15465 | 1345 |
| 1479 | 2300-18178WO | US03/15465 | 1346 |
| 1480 | 2300-18178WO | US03/15465 | 1347 |
| 1481 | 2300-18178WO | US03/15465 | 1348 |
| 1482 | 2300-18178WO | US03/15465 | 1349 |
| 1483 | 2300-18178WO | US03/15465 | 1350 |
| 1484 | 2300-18178WO | US03/15465 | 1351 |
| 1485 | 2300-18178WO | US03/15465 | 1352 |
| 1486 | 2300-18178WO | US03/15465 | 1353 |
| 1487 | 2300-18178WO | US03/15465 | 1354 |
| 1488 | 2300-18178WO | US03/15465 | 1355 |
| 1489 | 2300-18178WO | US03/15465 | 1356 |
| 1490 | 2300-18178WO | US03/15465 | 1357 |
| 1491 | 2300-18178WO | US03/15465 | 1358 |
| 1492 | 2300-18178WO | US03/15465 | 1359 |
| 1493 | 2300-18178WO | US03/15465 | 1360 |
| 1494 | 2300-18178WO | US03/15465 | 1361 |
| 1495 | 2300-18178WO | US03/15465 | 1362 |
| 1496 | 2300-18178WO | US03/15465 | 1363 |
| 1497 | 2300-18178WO | US03/15465 | 1364 |
| 1498 | 2300-18178WO | US03/15465 | 1365 |
| 1499 | 2300-18178WO | US03/15465 | 1366 |
| 1500 | 2300-18178WO | US03/15465 | 1367 |
| 1501 | 2300-18178WO | US03/15465 | 1368 |
| 1502 | 2300-18178WO | US03/15465 | 1369 |
| 1503 | 2300-18178WO | US03/15465 | 1370 |
| 1504 | 2300-18178WO | US03/15465 | 1371 |
| 1505 | 2300-18178WO | US03/15465 | 1372 |
| 1506 | 2300-18178WO | US03/15465 | 1373 |
| 1507 | 2300-18178WO | US03/15465 | 1374 |
| 1508 | 2300-18178WO | US03/15465 | 1375 |
| 1509 | 2300-18178WO | US03/15465 | 1376 |
| 1510 | 2300-18178WO | US03/15465 | 1377 |
| 1511 | 2300-18178WO | US03/15465 | 1378 |
| 1512 | 2300-18178WO | US03/15465 | 1379 |
| 1513 | 2300-18178WO | US03/15465 | 1380 |
| 1514 | 2300-18178WO | US03/15465 | 1381 |
| 1515 | 2300-18178WO | US03/15465 | 1382 |
| 1516 | 2300-18178WO | US03/15465 | 1383 |
| 1517 | 2300-18178WO | US03/15465 | 1384 |
| 1518 | 2300-18178WO | US03/15465 | 1385 |
| 1519 | 2300-18178WO | US03/15465 | 1386 |
| 1520 | 2300-18178WO | US03/15465 | 1387 |
| 1521 | 2300-18178WO | US03/15465 | 1388 |
| 1522 | 2300-18178WO | US03/15465 | 1389 |
| 1523 | 2300-18178WO | US03/15465 | 1390 |
| 1524 | 2300-18178WO | US03/15465 | 1391 |
| 1525 | 2300-18178WO | US03/15465 | 1392 |
| 1526 | 2300-18178WO | US03/15465 | 1393 |
| 1527 | 2300-18178WO | US03/15465 | 1394 |
| 1528 | 2300-18178WO | US03/15465 | 1395 |
| 1529 | 2300-18178WO | US03/15465 | 1396 |
| 1530 | 2300-18178WO | US03/15465 | 1397 |
| 1531 | 2300-18178WO | US03/15465 | 1398 |
| 1532 | 2300-18178WO | US03/15465 | 1399 |
| 1533 | 2300-18178WO | US03/15465 | 1400 |
| 1534 | 2300-18178WO | US03/15465 | 1401 |
| 1535 | 2300-18178WO | US03/15465 | 1402 |
| 1536 | 2300-18178WO | US03/15465 | 1403 |
| 1537 | 2300-18178WO | US03/15465 | 1404 |
| 1538 | 2300-18178WO | US03/15465 | 1405 |
| 1539 | 2300-18178WO | US03/15465 | 1406 |
| 1540 | 2300-18178WO | US03/15465 | 1407 |
| 1541 | 2300-18178WO | US03/15465 | 1408 |
| 1542 | 2300-18178WO | US03/15465 | 1409 |
| 1543 | 2300-18178WO | US03/15465 | 1410 |
| 1544 | 2300-18178WO | US03/15465 | 1411 |
| 1545 | 2300-18178WO | US03/15465 | 1412 |
| 1546 | 2300-18178WO | US03/15465 | 1413 |
| 1547 | 2300-18178WO | US03/15465 | 1414 |
| 1548 | 2300-18178WO | US03/15465 | 1415 |
| 1549 | 2300-18178WO | US03/15465 | 1416 |
| 1550 | 2300-18178WO | US03/15465 | 1417 |
| 1551 | 2300-18178WO | US03/15465 | 1418 |
| 1552 | 2300-18178WO | US03/15465 | 1419 |
| 1553 | 2300-18178WO | US03/15465 | 1420 |
| 1554 | 2300-18178WO | US03/15465 | 1421 |
| 1555 | 2300-18178WO | US03/15465 | 1422 |
| 1556 | 2300-18178WO | US03/15465 | 1423 |
| 1557 | 2300-18178WO | US03/15465 | 1424 |
| 1558 | 2300-18178WO | US03/15465 | 1425 |
| 1559 | 2300-18178WO | US03/15465 | 1426 |
| 1560 | 2300-18178WO | US03/15465 | 1427 |
| 1561 | 2300-18178WO | US03/15465 | 1428 |
| 1562 | 2300-18178WO | US03/15465 | 1429 |
| 1563 | 2300-18178WO | US03/15465 | 1430 |
| 1564 | 2300-18178WO | US03/15465 | 1431 |
| 1565 | 2300-18178WO | US03/15465 | 1432 |
| 1566 | 2300-18178WO | US03/15465 | 1433 |
| 1567 | 2300-18178WO | US03/15465 | 1434 |
| 1568 | 2300-18178WO | US03/15465 | 1435 |
| 1569 | 2300-18178WO | US03/15465 | 1436 |
| 1570 | 2300-18178WO | US03/15465 | 1437 |
| 1571 | 2300-18178WO | US03/15465 | 1438 |
| 1572 | 2300-18178WO | US03/15465 | 1439 |
| 1573 | 2300-18178WO | US03/15465 | 1440 |
| 1574 | 2300-18178WO | US03/15465 | 1441 |
| 1575 | 2300-18178WO | US03/15465 | 1442 |
| 1576 | 2300-18178WO | US03/15465 | 1443 |
| 1577 | 2300-18178WO | US03/15465 | 1444 |
| 1578 | 2300-18178WO | US03/15465 | 1445 |
| 1579 | 2300-18178WO | US03/15465 | 1446 |
| 1580 | 2300-18178WO | US03/15465 | 1447 |
| 1581 | 2300-18178WO | US03/15465 | 1448 |
| 1582 | 2300-18178WO | US03/15465 | 1449 |
| 1583 | 2300-18178WO | US03/15465 | 1450 |
| 1584 | 2300-18178WO | US03/15465 | 1451 |
| 1585 | 2300-18178WO | US03/15465 | 1452 |
| 1586 | 2300-18178WO | US03/15465 | 1453 |
| 1587 | 2300-18178WO | US03/15465 | 1454 |
| 1588 | 2300-18178WO | US03/15465 | 1455 |
| 1589 | 2300-18178WO | US03/15465 | 1456 |
| 1590 | 2300-18178WO | US03/15465 | 1457 |
| 1591 | 2300-18178WO | US03/15465 | 1458 |
| 1592 | 2300-18178WO | US03/15465 | 1459 |
| 1593 | 2300-18178WO | US03/15465 | 1460 |
| 1594 | 2300-18178WO | US03/15465 | 1461 |
| 1595 | 2300-18178WO | US03/15465 | 1462 |
| 1596 | 2300-18178WO | US03/15465 | 1463 |
| 1597 | 2300-18178WO | US03/15465 | 1464 |
| 1598 | 2300-18178WO | US03/15465 | 1465 |
| 1599 | 2300-18178WO | US03/15465 | 1466 |
| 1600 | 2300-18178WO | US03/15465 | 1467 |
| 1601 | 2300-18178WO | US03/15465 | 1468 |
| 1602 | 2300-18178WO | US03/15465 | 1469 |
| 1603 | 2300-18178WO | US03/15465 | 1470 |
| 1604 | 2300-18178WO | US03/15465 | 1471 |
| 1605 | 2300-18178WO | US03/15465 | 1472 |
| 1606 | 2300-18178WO | US03/15465 | 1473 |
| 1607 | 2300-18178WO | US03/15465 | 1474 |
| 1608 | 2300-18178WO | US03/15465 | 1475 |
| 1609 | 2300-18178WO | US03/15465 | 1476 |
| 1610 | 2300-18178WO | US03/15465 | 1477 |
| 1611 | 2300-18178WO | US03/15465 | 1478 |
| 1612 | 2300-18178WO | US03/15465 | 1479 |
| 1613 | 2300-18178WO | US03/15465 | 1480 |
| 1614 | 2300-18178WO | US03/15465 | 1481 |
| 1615 | 2300-18178WO | US03/15465 | 1482 |
| 1616 | 2300-18178WO | US03/15465 | 1483 |
| 1617 | 2300-18178WO | US03/15465 | 1484 |
| 1618 | 2300-18178WO | US03/15465 | 1485 |
| 1619 | 2300-18178WO | US03/15465 | 1486 |
| 1620 | 2300-18178WO | US03/15465 | 1487 |
| 1621 | 2300-18178WO | US03/15465 | 1488 |
| 1622 | 2300-18178WO | US03/15465 | 1489 |
| 1623 | 2300-18178WO | US03/15465 | 1490 |
| 1624 | 2300-18178WO | US03/15465 | 1491 |
| 1625 | 2300-18178WO | US03/15465 | 1492 |
| 1626 | 2300-18178WO | US03/15465 | 1493 |
| 1627 | 2300-18178WO | US03/15465 | 1494 |
| 1628 | 2300-18178WO | US03/15465 | 1495 |
| 1629 | 2300-18178WO | US03/15465 | 1496 |
| 1630 | 2300-18178WO | US03/15465 | 1497 |
| 1631 | 2300-18178WO | US03/15465 | 1498 |
| 1632 | 2300-18178WO | US03/15465 | 1499 |
| 1633 | 2300-18178WO | US03/15465 | 1500 |
| 1634 | 2300-18178WO | US03/15465 | 1501 |
| 1635 | 2300-18178WO | US03/15465 | 1502 |
| 1636 | 2300-18178WO | US03/15465 | 1503 |
| 1637 | 2300-18178WO | US03/15465 | 1504 |
| 1638 | 2300-18178WO | US03/15465 | 1505 |
| 1639 | 2300-18178WO | US03/15465 | 1506 |
| 1640 | 2300-18178WO | US03/15465 | 1507 |
| 1641 | 2300-18178WO | US03/15465 | 1508 |
| 1642 | 2300-18178WO | US03/15465 | 1509 |
| 1643 | 2300-18178WO | US03/15465 | 1510 |
| 1644 | 2300-18178WO | US03/15465 | 1511 |
| 1645 | 2300-18178WO | US03/15465 | 1512 |
| 1646 | 2300-18178WO | US03/15465 | 1513 |
| 1647 | 2300-18178WO | US03/15465 | 1514 |
| 1648 | 2300-18178WO | US03/15465 | 1515 |
| 1649 | 2300-18178WO | US03/15465 | 1516 |
| 1650 | 2300-18178WO | US03/15465 | 1517 |
| 1651 | 2300-18178WO | US03/15465 | 1518 |
| 1652 | 2300-18178WO | US03/15465 | 1519 |
| 1653 | 2300-18178WO | US03/15465 | 1520 |
| 1654 | 2300-18178WO | US03/15465 | 1521 |
| 1655 | 2300-18178WO | US03/15465 | 1522 |
| 1656 | 2300-18178WO | US03/15465 | 1523 |
| 1657 | 2300-18178WO | US03/15465 | 1524 |
| 1658 | 2300-18178WO | US03/15465 | 1525 |
| 1659 | 2300-18178WO | US03/15465 | 1526 |
| 1660 | 2300-18178WO | US03/15465 | 1527 |
| 1661 | 2300-18178WO | US03/15465 | 1528 |
| 1662 | 2300-18178WO | US03/15465 | 1529 |
| 1663 | 2300-18178WO | US03/15465 | 1530 |
| 1664 | 2300-18178WO | US03/15465 | 1531 |
| 1665 | 2300-18178WO | US03/15465 | 1532 |
| 1666 | 2300-18178WO | US03/15465 | 1533 |
| 1667 | 2300-18178WO | US03/15465 | 1534 |
| 1668 | 2300-18178WO | US03/15465 | 1535 |
| 1669 | 2300-18178WO | US03/15465 | 1536 |
| 1670 | 2300-18178WO | US03/15465 | 1537 |
| 1671 | 2300-18178WO | US03/15465 | 1538 |
| 1672 | 2300-18178WO | US03/15465 | 1539 |
| 1673 | 2300-18178WO | US03/15465 | 1540 |
| 1674 | 2300-18178WO | US03/15465 | 1541 |
| 1675 | 2300-18178WO | US03/15465 | 1542 |
| 1676 | 2300-18178WO | US03/15465 | 1543 |
| 1677 | 2300-18178WO | US03/15465 | 1544 |
| 1678 | 2300-18178WO | US03/15465 | 1545 |
| 1679 | 2300-18178WO | US03/15465 | 1546 |
| 1680 | 2300-18178WO | US03/15465 | 1547 |
| 1681 | 2300-18178WO | US03/15465 | 1548 |

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Source of Biological Materials and Overview of Polynucleotides Expressed by the Biological Materials

In order to identify genes that are differentially expressed in colon cancer, cDNA libraries were prepared from several different cell lines and tissue sources. Table 1 provides a summary of these libraries, including the shortened library name (used hereafter), the mRNA source used to prepared the cDNA library, the “nickname” of the library that is used in the tables below (in quotes), and the approximate number of clones in the library. cDNA libraries were prepared according to methods well known in the art, and the sequences of the cDNA inserts were determined using well known methods.

TABLE 1
Description of cDNA Libraries

| | | Number |
|---|---|---|
| | | of |
| Library | Description | Clones |
| 1 | Human Colon Cell Line Km12 L4: High Metastatic | 308731 |
| | Potential (derived from Km12C) | |
| 2 | Human Colon Cell Line Km12C: Low Metastatic | 284771 |
| | Potential | |
| 3 | Human Breast Cancer Cell Line MDA-MB-231: High | 326937 |
| | Metastatic Potential; micromets in lung | |
| 4 | Human Breast Cancer Cell Line MCF7: Non- | 318979 |
| | Metastatic | |
| 8 | Human Lung Cancer Cell Line MV-522: High | 223620 |
| | Metastatic Potential | |
| 9 | Human Lung Cancer Cell Line UCP-3: Low | 312503 |
| | Metastatic Potential | |
| 12 | Human microvascular endothelial cells (HMEC) - | 41938 |
| | UNTREATED (PCR (OligodT) cDNA library) | |
| 13 | Human microvascular endothelial cells (HMEC) - | 42100 |
| | bFGF TREATED (PCR (OligodT) cDNA library) | |
| 14 | Human microvascular endothelial cells (HMEC) - | 42825 |
| | VEGF TREATED (PCR (OligodT) cDNA library) | |
| 15 | Normal Colon - UC#2 Patient (MICRODISSECTED | 282718 |
| | PCR (OligodT) cDNA library) | |
| 16 | Colon Tumor - UC#2 Patient (MICRODISSECTED | 298829 |
| | PCR (OligodT) cDNA library) | |
| 17 | Liver Metastasis from Colon Tumor of UC#2 Patient | 303462 |
| | (MICRODISSECTED PCR (OligodT) cDNA library) | |
| 18 | Normal Colon - UC#3 Patient (MICRODISSECTED | 36216 |
| | PCR (OligodT) cDNA library) | |
| 19 | Colon Tumor - UC#3 Patient (MICRODISSECTED | 41388 |
| | PCR (OligodT) cDNA library) | |
| 20 | Liver Metastasis from Colon Tumor of UC#3 Patient | 30956 |
| | (MICRODISSECTED PCR (OligodT) cDNA library) | |
| 21 | GRRpz Cells derived from normal prostate epithelium | 164801 |
| 22 | WOca Cells derived from Gleason Grade 4 prostate | 162088 |
| | cancer epithelium | |
| 23 | Normal Lung Epithelium of Patient #1006 | 306198 |
| | (MICRODISSECTED PCR (OligodT) cDNA library) | |
| 24 | Primary tumor, Large Cell Carcinoma of Patient | 309349 |
| | #1006 (MICRODISSECTED PCR (OligodT) cDNA | |
| | library) | |
| 25 | Normal Prostate Epithelium from Patient 1F97-26811 | 279437 |
| 26 | Prostate Cancer Epithelium Gleason 3 + 3 Patient | 269366 |
| | IF97-26811 | |

The KM12L4 cell line is derived from the KM12C cell line (Morikawa, et al., Cancer Research (1988) 48:6863). The KM12C cell line, which is poorly metastatic (low metastatic) was established in culture from a Dukes' stage B₂ surgical specimen (Morikawa et al. Cancer Res. (1988) 48:6863). The KML4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246).

The MDA-MB-231 cell line was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. These cell lines are well-recognized in the art as models for the study of human breast and lung cancer (see, e.g., Chandrasekaran et al., Cancer Res. (1979) 39:870; Gastpar et al., J Med Chem (1998) 41:4965; Ranson et al., Br J Cancer (1998) 77:1586; Kuang et al., Nucleic Acids Res (1998) 26:1116. The samples of libraries 15-20 are derived from two different patients (UC#2 and UC#3). The GRRpz and WOca cell lines were provided by Dr. Donna M. Peehl, Department of Medicine, Stanford University School of Medicine. GRRpz was derived from normal prostate epithelium. The WOca cell line is a Gleason Grade 4 cell line.

Each of the libraries is composed of a collection of cDNA clones that in turn are representative of the mRNAs expressed in the indicated mRNA source. In order to facilitate the analysis of the millions of sequences in each library, the sequences were assigned to clusters. The concept of “cluster of clones” is derived from a sorting/grouping of cDNA clones based on their hybridization pattern to a panel of roughly 300 7 bp oligonucleotide probes (see Drmanac et al., Genomics (1996) 37(1):29). Random cDNA clones from a tissue library are hybridized at moderate stringency to 300 7 bp oligonucleotides. Each oligonucleotide has some measure of specific hybridization to that specific clone. The combination of 300 of these measures of hybridization for 300 probes equals the “hybridization signature” for a specific clone. Clones with similar sequence will have similar hybridization signatures. By developing a sorting/grouping algorithm to analyze these signatures, groups of clones in a library can be identified and brought together computationally. These groups of clones are termed “clusters”.

Depending on the stringency of the selection in the algorithm (similar to the stringency of hybridization in a classic library cDNA screening protocol), the “purity” of each cluster can be controlled. For example, artifacts of clustering may occur in computational clustering just as artifacts can occur in “wet-lab” screening of a cDNA library with 400 bp cDNA fragments, at even the highest stringency. The stringency used in the implementation of cluster herein provides groups of clones that are in general from the same cDNA or closely related cDNAs. Closely related clones can be a result of different length clones of the same cDNA, closely related clones from highly related gene families, or splice variants of the same cDNA.

Differential expression for a selected cluster was assessed by first determining the number of cDNA clones corresponding to the selected cluster in the first library (Clones in 1^{st}), and the determining the number of cDNA clones corresponding to the selected cluster in the second library (Clones in 2^{nd}). Differential expression of the selected cluster in the first library relative to the second library is expressed as a “ratio” of percent expression between the two libraries. In general, the “ratio” is calculated by: 1) calculating the percent expression of the selected cluster in the first library by dividing the number of clones corresponding to a selected cluster in the first library by the total number of clones analyzed from the first library; 2) calculating the percent expression of the selected cluster in the second library by dividing the number of clones corresponding to a selected cluster in a second library by the total number of clones analyzed from the second library; 3) dividing the calculated percent expression from the first library by the calculated percent expression from the second library. If the “number of clones” corresponding to a selected cluster in a library is zero, the value is set at 1 to aid in calculation. The formula used in calculating the ratio takes into account the “depth” of each of the libraries being compared, i.e., the total number of clones analyzed in each library.

As a result of this library comparison, 17 polynucleotides, listed as SEQ ID NOS:1, 3, 5, 7, 9, 11-13, 15, 16, 18, 20, 22, 24, 26, 27 and 29 in the accompanying Sequence Listing and summarized in Table 2, were identified as corresponding to genes differentially expressed in colon cancer patient tissues. Table 2 provides: 1) the sequence identification number (“SEQ ID NO of polynucleotide”) assigned to each sequence for use in the present specification; 2) the cluster identification number (“CLUSTER”); 3) the Candidation Idnetification number; 4) ththe CHIR number (which serves as tha cross-reference to antisense oligos discussed below), with, for examplek CHIR7 having corresponding oligos CHIR7-2AS (antibsense) and CHIR7-RC (reverse control); 5) the sequence name (“SEQ NAME”) used as an internal identifier of the sequence; 6) the name assigned to the clone from which the sequence was isolated (“CLONE ID”); 7) the first nucleotide of the start and stop codons of identified open reading frames (“ORF start” and “ORF stop”); and 8) the sequence identification number (“SEQ ID NO of encoded polypeptide”) assigned to the encoded polypeptide, where appropriate. Because the provided polynucleotides represent partial mRNA transcripts, two or more polynucleotides of the invention may represent different regions of the same mRNA transcript and the same gene. Thus, if two or more sequences are identified as belonging to the same clone, then either sequence can be used to obtain the full-length mRNA or gene.

TABLE 2
Polynucleotide sequence identificaton and characterization

| | | | | | | | SEQ ID NO |
|---|---|---|---|---|---|---|---|
| SEQ | | Candidate | | SEQ | ORF | | of encoded |
| BID NO | CLUSTER | ID | CHIR | NAME | start | stop | polypeptide |
| 1 | 719 | 196 | CHIR-7 | SK1 | 21 | 396 | 2 |
| 3 | 9083 | 181 | CHIR-8 | SK2 | 219 | 693 | 4 |
| 5 | 115762 | 188 | CHIR-16 | SK5 | 5 | 1760 | 6 |
| 7 | 1665 | 195 | CHIR-9 | 1665 long | 78 | 642 | 8 |
| 9 | 1665 | 195 | CHIR-9 | 1665 short | 79 | 232 | 10 |
| 11 | 2334 | | | SK8 partial | | | |
| 12 | 2334 | | | SK8 full | | | |
| | | | | length | | | |
| 13 | 3376 | 118 | CHIR-11 | SK19 | 79 | 376 | 14 |
| 15 | 376130 | | | Junc2 | 181, 363, | 361, 542, 911 | |
| | | | | | 731 | | |
| 16 | 402380 | 202 | CHIR-33 | XD4 | 16 | 538 | 17 |
| 18 | 726682 | 198 | CHIR-43 | XD1 | 2 | 551 | 19 |
| 20 | 552930 | 174 | CHIR-42 | XD7 | 240 | 585 | 21 |
| 22 | 454001 | 161 | CHIR-29 | XD10 | 53 | 1700 | 23 |
| 24 | 378805 | 163 | CHIR-31 | XD11 | 10 | 400 | 25 |
| 26 | 374641 | 160 | CHIR-32 | 374641 long | 33, 420 | 183, 615 | |
| | | | | (Junc4) | | | |
| 27 | 374641 | 160 | CHIR-32 | 374641 short | 324 | 519 | 28 |
| | | | | (XD6) | | | |
| 29 | 374641 | 160 | CHIR-32 | 374641 | 40, 388 | 190, 583 | |
| | | | | electronic | | | |

Table 3 summarizes polynucleotides that correspond to genes differentially expressed in colon tissue from a single patient.

TABLE 3

| SEQ | | Normal | Tumor | High Met | Tumor/ | High Met/ | High Met/ |
|---|---|---|---|---|---|---|---|
| ID | | (Lib15) | (Lib16) | (Lib17) | Normal | Normal | Tumor |
| NO | CLUSTER | Clones | Clones | Clones | (Lib16/Lib15) | (Lib17/Lib15) | (Lib17/Lib16) |
| 1 | 719 | 0 | 20 | 27 | 20 | 27 | 1 |
| 3 | 9083 | 0 | 10 | 14 | 10 | 14 | 1 |
| 5 | 115762 | 0 | 6 | 7 | 6 | 7 | 1 |
| 7 | 1665 | 4 | 14 | 20 | 3.5 | 5 | 1 |
| 12 | 2334 | 0 | 6 | 1 | 6 | 1 | 0 |
| 13 | 3376 | 3 | 20 | 19 | 7 | 6 | 1 |
| 15 | 376130 | 0 | 9 | 15 | 9 | 15 | 2 |
| 16 | 402380 | 0 | 15 | 2 | 15 | 2 | 0 |
| 18 | 726682 | 0 | 52 | 0 | 52 | 0 | 0 |
| 20 | 552930 | 1 | 14 | 2 | 14 | 2 | 0 |
| 22 | 454001 | 0 | 8 | 13 | 8 | 13 | 2 |
| 24 | 378805 | 1 | 12 | 12 | 12 | 12 | 1 |
| 26 | 374641 | 9 | 47 | 129 | 5 | 14 | 3 |

### Example 2

### Analysis and Characterization of Polynucleotides of the Invention

Several of the provided polynucleotides contain one or more putative open reading frames (ORFs) encoding a gene product. The start and stop sites for these ORFs are listed in Table 2.

SEQ ID NO:15 contains three ORFs. The first ORF extends from nucleotide 181 to nucleotide 361. The second ORF extends from nucleotide 363 to nucleotide 542. The third ORF extends from nucleotide 731 to nucleotide 911.

SEQ ID NO:26 contains a 39-nucleotide insertion sequence (from nucleotide 269 to nucleotide 307) and two ORFs. The first ORF extends from nucleotide 33 to nucleotide 183. The second ORF extends from nucleotide 420 to nucleotide 615.

SEQ ID NO:29 is an electronic sequence according to the 5′-RACE result and contains two ORFs. The first ORF extends from nucleotide 40 to nucleotide 190. The second ORF extends from nucleotide 388 to nucleotide 583.

### Example 3

### Members of Protein Families

Translations of the provided polynucleotides were aligned with amino acid profiles that define either protein families or common motifs. Several of the polynucleotides of the invention were found to encode polypeptides having characteristics of a polypeptide belonging to a known protein family (and thus represent new members of these protein families) and/or comprising a known functional domain. Similarity between a query sequence and a protein family or motif was determined by (a) comparing the query sequence against the profile and/or (b) aligning the query sequence with the members of the family or motif.

Each of the profile hits is described in more detail below. Table 4 provides the corresponding SEQ ID NO of the provided polynucleotides that encode gene products with similarity or identity to the profile sequences. Similarity (strong or weak) is also noted in Table 4. The acronyms for the profiles (provided in parentheses) are those used to identify the profile in the Pfam and Prosite databases. The Pfam database can be accessed through any of the following URLS: http://pfam.wustl.edu/index.html; http://www.sanger.ac.uk/Software/Pfam/; and http://www.cgr.ki.se/Pfam/. The Prosite database can be accessed at http://www.expasy.ch/prosite/. The public information available on the Pfam and Prosite databases regarding the various profiles, including but not limited to the activities, function, and consensus sequences of various proteinss families and protein domains, is incorporated herein by reference.

TABLE 4
Profile hits.

| SEQ | | | | |
|---|---|---|---|---|
| ID NO | CLUSTER | Profile | Description | Similarity |
| 1 | 719 | | Glycosyl hydrolase | weak |
| 3 | 9083 | ANK | Ankyrin repeats | strong |
| 5 | 115762 | 7tm_1 | 7 transmembrane receptor | weak |
| | | | (rhodopsin family) | |
| 11 | 2334 | EFhand | EF-hand | strong |
| 12 | 2334 | Efhand | EF-hand | strong |
| 15 | 376130 | | Endogenous retrograde | |
| | | | protease/integrase | |
| 16 | 402380 | Rrm | RNA recognition motif. | |
| | | | (aka RRM, RBD, | |
| | | | or RNP domain) | |

Glycosyl hydrolase family 5 (GLYCOSYL_HYDROL_F5; Pfam Accession No. PS00659; PDOC00565). SEQ ID NO:1 corresponds to a gene encoding a polypeptide having homology to polypeptides of the glycosyl hydrolase family 5 (Henrissat Biochem. J. (1991) 280:309-316) (also known as the cellulase family A (Henrissat et al. Gene (1989) 81:83-95)). The members of this family participate in the degradation of cellulose and xylans, and are generally found in bacteria, fungi, and yeast. The consensus pattern for members of this family is: [LIV]-[LIVMFYWGA](2)-[DNEQG]-[LIVMGST]-x-N-E-[PV]-[RHDNSTLIVFY] (where E is a putative active site residue).

SEQ ID NO:1 corresponds to a gene encoding a member of one of the families of glycosyl hydrolases (Henrissat et al. Biochem. J. (1993) 293:781-788). These enzymes contain at least one conserved glutamic acid residue (or aspartic acid residue) which has been shown to be directly involved in glycosidic bond cleavage by acting as a nucleophile.

Ank Repeats (ANK; Pfam Accession No. PF0023). SEQ ID NO:3 corresponds to a gene encoding an Ank repeat-containing protein. The ankyrin motif is a 33 amino acid sequence named after the protein ankyrin which has 24 tandem 33-amino-acid motifs. Ank repeats were originally identified in the cell-cycle-control protein cdc10 (Breeden et al., Nature (1987) 329:651). Proteins containing ankyrin repeats include ankyrin, myotropin, I-kappaB proteins, cell cycle protein cdc10, the Notch receptor (Matsuno et al., Development (1997) 124(21):4265); G9a (or BAT8) of the class III region of the major histocompatibility complex (Biochem J. 290:811-818, 1993), FABP, GABP, 53BP2, Lin12, glp-1, SW14, and SW16. The functions of the ankyrin repeats are compatible with a role in protein-protein interactions (Bork, Proteins (1993) 17(4):363; Lambert and Bennet, Eur. J. Biochem. (1993) 211:1; Kerr et al., Current Op. Cell Biol. (1992) 4:496; Bennet et al., J. Biol. Chem. (1980) 255:6424).

Seven Transmembrane Integral Membrane Proteins—Rhodopsin Family (7tm_{—}1: Pfam Accession No. PF00001). SEQ ID NO:3 corresponds to a gene encoding a polypeptide that is a member of the seven transmembrane (7tm) receptor rhodopsin family. G-protein coupled receptors of the (7tm) rhodopsin family (also called R7G) are an extensive group of hormones, neurotransmitters, and light receptors which transduce extracellular signals by interaction with guanine nucleotide-binding (G) proteins (Strosberg A. D. Eur. J. Biochem. (1991) 196:1, Kerlavage A. R. Curr. Opin. Struct. Biol. (1991) 1:394, Probst, et al., DNA Cell Biol. (1992) 11:1, Savarese, et al., Biochem. J. (1992) 283:1, http://www.gcrdb.uthscsa.edu/, http://swift.embl-heidelberg.de/7tm/. The consensus pattern that contains the conserved triplet and that also spans the major part of the third transmembrane helix is used to detect this widespread family of proteins:

[GSTALIVMFYWC]-[GSTANCPDE]-{EDPKRH}-x(2)-
[LIVMNQGA]-x(2)-[LIVMFT]-[GSTANC]-[LIVMFYWSTAC]-
[DENH]-R-[FYWCSH]-x(2)-[LIVM].

[GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM].

EF Hand (EFhand: Pfam Accession No. PF00036). SEQ ID NOS:11 and 12 correspond to genes encoding a protein in the family of EF-hand proteins. Many calcium-binding proteins belong to the same evolutionary family and share a type of calcium-binding domain known as the EF-hand (Kawasaki et al., Protein. Prof. (1995) 2:305-490). This type of domain consists of a twelve residue loop flanked on both sides by a twelve residue alpha-helical domain. In an EF-hand loop the calcium ion is coordinated in a pentagonal bipyramidal configuration. The six residues involved in the binding are in positions 1, 3, 5, 7, 9 and 12; these residues are denoted by X, Y, Z, —Y, —X and -Z. The invariant Glu or Asp at position 12 provides two oxygens for liganding Ca (bidentate ligand). The consensus pattern includes the complete EF-hand loop as well as the first residue which follows the loop and which seem to always be hydrophobic: D-x-[DNS]-{ILVFYW}-[DENSTG]-[DNQGHRK]-{GP}-[LIVMC]-[DENQSTAGC]-x(2)-[DE]-[LIVMFYW].

Endogenous retroviral protease/integrase. SEQ ID NO:15 corresponds to a gene encoding a polypeptide having a domain homologous to a human endogenous retrovirus protease/integrase domain of a retroviral pol protein.

RNA Recognition Motif (rrm: Pfam Accession No. PF00076). SEQ ID NO:16 corresponds to a gene encoding an RNA recognition motif, also known as an RRM, RBD, or RNP domain. This domain, which is about 90 amino acids long, is contained in eukaryotic proteins that bind single-stranded RNA (Bandziulis et al. Genes Dev. (1989) 3:431-437; Dreyfuss et al. Trends Biochem. Sci. (1988) 13:86-91). Two regions within the RNA-binding domain are highly conserved: the first is a hydrophobic segment of six residues (which is called the RNP-2 motif), the second is an octapeptide motif (which is called RNP-1 or RNP-CS). The consensus pattern is: [RK]-G-{EDRKHPCG}-[AGSCI]-[FY]-[LIVA]-x-[FYLM].

### Example 4

### Detection and Quantification of Polynucleotides of the Invention

The polynucleotides of the invention were detected and quantified in patient tissue samples by reverse transcriptase PCR (RT-PCR). Total RNA amplifications were performed using the LightCycler™ thermal cycling system (Roche Diagnostics) in a standard PCR reaction containing the provided primers and the dsDNA-binding dye SYBR Green I. PCR amplifacaiotn was monitored by fluroescence dye SYBR Green I, which fluroesces only when bound to double-stranded DNA. The specific of the products was verified by melting curve analysis.

Standard Preparation. 1 μg human placenta total RNA (Clontech, Palo Alto, Calif.) was reverse-transcribed at 42° C. for 1 hour then heated at 94° C. for 5 minutes in a total reaction volume of 20 μl (1st-Strand™ cDNA Synthesis Kit, Clontech). The reaction mix was used as 1× template standard. Serial dilutions from 1× template standard were then prepared: 10⁻¹×, 10⁻²×, 10⁻³×, 10⁻⁴×, 10⁻⁵×, 10⁻⁶× template standards.

Total RNA Sample Preparation. The patient tissue samples were shipped in frozen TRIZOL reagent. The samples were homogenized in TRIZOL reagent. Chloroform was then added to isolate RNA, followed by RNA precipitation with isopropanol. The RNA precipitates were washed with 75% ethanol, dried in air, then dissolved in RNase-free distilled water. Before reverse-transcription, RNA samples were treated with DNase I (RNase-free) (2 U/μl, Ambion, Austin, Tex.) and cleaned up using RNeasy Mini Kit (Qiagen, Santa Clarita, Calif.).

RT-PCR. Total RNA samples were reverse-transcribed with oligo-dT₁₈ primer (1st-Strand™ cDNA Synthesis Kit, Clontech). PCR was performed using the following gene-specific primers:

| | | | |
|---|---|---|---|
| SK1: | forward primer | 5′-AGGAGTTTCTGAGGACCATGCAC-3′ | (SEQ ID NO:30) |
| | reverse primer | 5′-TCAAGGGTTGGGGATACACACG-3′ | (SEQ ID NO:31) |
| SK2: | forward primer | 5′-CTTGCTTGCTTTCTTCTCTGGC-3′ | (SEQ ID NO:32) |
| | reverse primer | 5′-AGTCTGGAAATCCACATGACCAAG-3′ | (SEQ ID NO:33) |
| SK5: | forward primer | 5′-CCCAATGAGGAACCTAAAGTTGC-3′ | (SEQ ID NO:34) |
| | reverse primer | 5′-GGTGCCAAATCTGGACTCTTGTC-3′ | (SEQ ID NO:35) |
| 1665: | forward primer | 5′-GATCCATTTTCAGCAGTGCTCTG-3′ | (SEQ ID NO:36) |
| | reverse primer | 5′-CAGTGTTCACAGAAGGGGTACTCAC-3′ | (SEQ ID NO:37) |
| SK8: | forward primer | 5′-ACGAGAGCGACACGGACAAG-3′ | (SEQ ID NO:38) |
| | reverse primer | 5′-TCTGAGGCTGTGGCAGGTGC-3′ | (SEQ ID NO:39) |
| SK19: | forward primer | 5′-CCAGTCTTTGCCAACTCGTGC-3′ | (SEQ ID NO:40) |
| | reverse primer | 5′-TTCGATCTTCAAACTGTGCCTTG-3′ | (SEQ ID NO:41) |
| Junc2: | forward primer | 5′-TTGGCAACCAGACCAGCATC-3′ | (SEQ ID NO:42) |
| | reverse primer | 5′-TTTCCCATAGGTGTGAGTGGCG-3′ | (SEQ ID NO:43) |
| XD4: | forward primer | 5′-GACTGGTGTTTTGTTCGGGGTC-3′ | (SEQ ID NO:44) |
| | reverse primer | 5′-TTTGTCCAAGGCTGCATGGTC-3′ | (SEQ ID NO:45) |
| XD1: | forward primer | 5′-TGCCCTGGTTAAGCCAGAAGTC-3′ | (SEQ ID NO:46) |
| | reverse primer | 5′-AGCTTCACTTTGGTCTTGACGG-3′ | (SEQ ID NO:47) |
| XD7: | forward primer | 5′-GGTCATCTGCATCAAGGTTGGC-3′ | (SEQ ID NO:48) |
| | reverse primer | 5′-GGTTCGTAACCGTGACTTCAGG-3′ | (SEQ ID NO:49) |
| XD10: | forward primer | 5′-GCATCCTTTTCCAGTCTTCCG-3′ | (SEQ ID NO:50) |
| | reverse primer | 5′-TGCAGCAAACATGCCTGAGC-3′ | (SEQ ID NO:51) |
| XD11: | forward primer | 5′-TGTTCCACGAGCAAAGCATGTG-3′ | (SEQ ID NO:52) |
| | reverse primer | 5′-ATCCTTCTTCCACTCCCGCTTC-3′ | (SEQ ID NO:53) |
| 37641: | forward primer | 5′-TCGGCTTGACTACACTGTGTGG-3′ | (SEQ ID NO:54) |
| | reverse primer | 5′-TACAAAGACCACTGGGAGGCTG-3′ | (SEQ ID NO:55) |
| β-actin: | forward primer | 5′-CGGGAAATCGTGCGTGACATTAAG-3′ | (SEQ ID NO:56) |
| | reverse primer | 5′-TGATCTCCTTCTGCATCCTGTCGG-3′ | (SEQ ID NO:57) |
| GAPDH: | forward primer | 5′-TTTGGCTACAGCAACAGGGTG-3′ | (SEQ ID NO:58) |
| | reverse primer | 5′-TGTGAGGAGGGGAGATTCAGTG-3′ | (SEQ ID NO:59) |

β-actin and GAPDH were used as positive controls. All PCR products are 150-250 bp. The 20-μl PCR reaction mix in each LightCycler™ capillary contained 2 μl of 10×PCR buffer II, 3 mM MgCl₂ (Perkin-Elmer, Foster City, Calif.), 140 μM dNTP, 1:50000 of SYBR Green I, 0.25 mg/ml BSA, 1 unit of Taq polymerase (Boehringer Mannheim, Indianapolis, Ind.), 0.175 μM each primer, 2 μl of RT reaction mix. The PCR amplification began with 20-second denaturation at 95° C., followed by 45 cycles of denaturation at 95° C. for 5 seconds, annealing at 60° C. for 1 second and extension at 72° C. for 30 seconds. At the end of final cycle, PCR products were annealed at 60° C. for 5 seconds, then slowly heated to 95° C. at 0.2° C./second, to measure melting curve of specific PCR products. All experiments were performed in duplicate.

Data analysis was performed using LightCycler™ software (Roche Diagnostics) with quantification and melting curve options. Fluorescence is normalized relative to positive and negative controls.

Overexpression of genes in colon cancer patient whole tissue. Results provided in the tables below include fluoresence data for polynucleotides isolated from colon tissue samples that were harvested directly, not microdissected (i.e., whole tissue), and amplified using the indicated primers. Normal, primary tumor and metastatic cell types are denoted as N, PT and Met, respectively. Overexpression was determined by comparing either metastatic cells or primary tumor cells, or both, to normal cells. The results for each gene corresponding to the indicated clusters in each patient sample are summarized in the tables below. All values are adjusted to levels relative to beta-actin control.

Cluster#719 (SK1): overexpression detected in
4 of 6 patients (67%)

| Patients | N | PT | MET |
|---|---|---|---|
| UC#1 | 0.022 | 0.117 | 0.364 |
| UC#2 | 0.121 | 0.109 | 0.142 |
| UC#4 | 0.083 | 0.053 | 0.078 |
| UC#7 | 0.042 | 0.199 | 0.145 |
| UC#8 | 0.215 | 0.515 | 0.794 |
| UC#9 | 0.233 | 0.585 | 0.613 |

Cluster#9083 (SK2): overexpression inf 3 or 4
patients (75%)

| Patients | N | PT | MET |
|---|---|---|---|
| UC#1 | 0.0021 | 0.0013 | 0.0078 |
| UC#2 | 0.008 | 0.012 | 0.014 |
| UC#4 | 0.0021 | 0.0022 | 0.0026 |
| UC#7 | 0.0009 | 0.0021 | 0.0039 |

Cluster#115762 (SK5): overexpression in
5 of 6 patients (83%)

| Patients | N | PT | MET |
|---|---|---|---|
| UC#1 | 0.0053 | 0.0159 | 0.044 |
| UC#2 | 0.0195 | 0.0174 | 0.0269 |
| UC#4 | 0.022 | 0.033 | 0.034 |
| UC#7 | 0.013 | 0.028 | 0.025 |
| UC#8 | 0.0275 | 0.105 | 0.143 |
| UC#9 | 0.0336 | 0.0595 | 0.0541 |

Cluster#1665: overexpression in 4 of 6
patients (67%)

| Patients | N | PT | MET |
|---|---|---|---|
| UC#1 | 0.00006 | 0.0003 | 0.002 |
| UC#2 | 0.0015 | 0.001 | 0.0012 |
| UC#4 | 0.0016 | 0.0013 | 0.0016 |
| UC#7 | 0.00003 | 0.0003 | 0.0012 |
| UC#8 | 0.0016 | 0.0122 | 0.0154 |
| UC#9 | 0.006 | 0.057 | 0.097 |

Cluster#2334 (SK8): overexpression in 4
of 6 patients (67%)

| Patients | N | PT | MET |
|---|---|---|---|
| UC#1 | 0.011 | 0.022 | 0.017 |
| UC#2 | 0.0266 | 0.0317 | 0.026 |
| UC#4 | 0.02 | 0.006 | 0.01 |
| UC#7 | 0.046 | 0.093 | 0.042 |
| UC#8 | 0.042 | 0.168 | 0.472 |
| UC#9 | 0.208 | 0.322 | 0.29 |

Cluster#3376 (SK19): overexpression in 4
of 6 patients (67%)

| Patients | N | PT | MET |
|---|---|---|---|
| UC#1 | 0.00018 | 0.00042 | 0.0012 |
| UC#2 | 0.002 | 0.0025 | 0.0016 |
| UC#4 | 0.0013 | 0.0012 | 0.002 |
| UC#7 | 0.00024 | 0.00055 | 0.00062 |
| UC#8 | 0.0003 | 0.00127 | 0.0023 |
| UC#9 | 0.001 | 0.0075 | 0.009 |

Cluster#376130 (Junc2): overexpression
in 3 of 4 patients (75%)

| Patients | N | PT | MET |
|---|---|---|---|
| UC#1 | 0.00871 | 0.0111 | 0.0142 |
| UC#2 | 0.000567 | 0.00663 | 0.0163 |
| UC#4 | 0.000107 | 0.00048 | 0.000237 |
| UC#7 | 0.0000401 | 0.000259 | 0.00159 |

Cluster#402380 (XD4): overexpression in
2 of 4 patients (50%)

| Patients | N | PT | MET |
|---|---|---|---|
| UC#1 | 0.0763 | 0.123 | 0.2 |
| UC#2 | 0.0867 | 0.0629 | 0.069 |
| UC#4 | 0.0735 | 0.0672 | 0.0664 |
| UC#7 | 0.0559 | 0.112 | 0.139 |

Cluster#726682 (XD1): overexpression
in 0 of 4 patients

| Patients | N | PT | MET |
|---|---|---|---|
| UC#1 | 0.0679 | 0.0822 | 0.136 |
| UC#2 | 0.175 | 0.124 | 0.147 |
| UC#4 | 0.2 | 0.145 | 0.145 |
| UC#7 | 0.108 | 0.144 | 0.114 |

Cluster#552930 (XD7): overexpression in
1 of 4 patients (25%)

| Patients | N | PT | MET |
|---|---|---|---|
| UC#1 | 0.018 | 0.019 | 0.0902 |
| UC#2 | 0.204 | 0.161 | 0.212 |
| UC#4 | 0.299 | 0.25 | 0.238 |
| UC#7 | 0.246 | 0.409 | 0.248 |

Cluster#454001 (XD10): overexpression
in 2 of 4 patients)

| Patients | N | PT | MET |
|---|---|---|---|
| UC#1 | 0.0197 | 0.0363 | 0.0587 |
| UC#2 | 0.0514 | 0.0451 | 0.069 |
| UC#4 | 0.0587 | 0.0889 | 0.096 |
| UC#7 | 0.0342 | 0.1 | 0.0705 |

Cluster#378805 (XD11): overexpression
in 1 of 4 patients)

| Patients | N | PT | MET |
|---|---|---|---|
| UC#1 | 0.00117 | 0.00269 | 0.00697 |
| UC#2 | 0.00864 | 0.00371 | 0.00672 |
| UC#4 | 0.0098 | 0.00525 | 0.00497 |
| UC#7 | 0.00912 | 0.00989 | 0.0127 |

Cluster#374641: overexpression in 3 of 4
patients (75%)

| Patients | N | PT | MET |
|---|---|---|---|
| UC#1 | 0.0124 | 0.163 | 0.0947 |
| UC#2 | 0.28 | 0.317 | 0.544 |
| UC#4 | 0.685 | 1.809 | 1.996 |
| UC#7 | 0.569 | 1.714 | 1.073 |

Overexpression of genes in colon cancer patient epithelium. Results provided in the tables below include fluorescence data for polynucleotides isolated from colon epithelial cells that were prepared by the epithelial shakeoff method to obtain >97% pure epithelium without stroma. Normal, precancerous (adenomatous polyp), and primary tumor cell types are denoted as N, polyp and PT, respectively. Overexpression was determined by comparing either primary tumor cells or precancerous cells, or both, to normal cells. All values are adjusted to levels relative to beta-actin control.

Cluster#719 (SK1): overexpression in 4
of 4 patients (100%)

| Patients | N | Polyp | PT |
|---|---|---|---|
| UW#17 | 0.0924 | 0.117 | N/A |
| UW#18 | 0.0864 | N/A | 0.327 |
| UW#19 | 0.151 | N/A | 0.227 |
| UW#20 | 0.0624 | 0.162 | 0.164 |

Cluster#115762 (SK5): overexpression
in 4 of 4 patients (100%).

| Patients | N | Polyp | PT |
|---|---|---|---|
| UW#17 | 0.00724 | 0.0122 | N/A |
| UW#18 | 0.0156 | N/A | 0.111 |
| UW#19 | 0.0158 | N/A | 0.0461 |
| UW#20 | 0.00728 | 0.0187 | 0.0306 |

Cluster#1665: overexpression in 4 of 4
patients (100%)

| Patients | N | Polyp | PT |
|---|---|---|---|
| UW#17 | 0.0041 | 0.0306 | N/A |
| UW#18 | 0.0029 | N/A | 0.0357 |
| UW#19 | 0.0045 | N/A | 0.0357 |
| UW#20 | 0.0028 | 0.025 | 0.047 |

Cluster#2334 (SK8) overexpressed in 1
of 4 patients (25%)

| Patients | N | Polyp | PT |
|---|---|---|---|
| UW#17 | 0.1835 | 0.041 | N/A |

Cluster#2334 (SK8) overexpressed in 1
of 4 patients (25%)

| Patients | N | Polyp | PT |
|---|---|---|---|
| UW#18 | 0.0638 | N/A | 0.0927 |
| UW#19 | 0.04 | N/A | 0.04 |
| UW#20 | 0.2236 | 0.0576 | 0.0454 |

Cluster#3376 (SK19) overexpressed in 4 of 4 patients (100%)

| Patients | N | Polyp | PT |
|---|---|---|---|
| UW#17 | 0.0053 | 0.012 | N/A |
| UW#18 | 0.0028 | N/A | 0.0084 |
| UW#19 | 0.003 | N/A | 0.0135 |
| UW#20 | 0.0023 | 0.023 | 0.012 |

### Example 5

### Northern Blot Analysis

Differential gene expression in cancerous colon cells can be further confirmed by other techniques, such as Northern blot analysis. Northern analysis can be accomplished by methods well-known in the art. Briefly, rapid-Hyb buffer (Amersham Life Science, Little Chalfont, England) with 5 mg/ml denatured single stranded sperm DNA is pre-warmed to 65° C. and human colon tumor total RNA blots (Invitrogen, Carlsbad, Calif.) are pre-hybridized in the buffer with shaking at 65° C. for 30 minutes. Gene-specific DNA probes (50 ng per reaction) labeled with [α-32P]dCTP (3000 Ci/mmol, Amersham Pharmacia Biotech Inc., Piscataway, N.J.) (Prime-It RmT Kit, Stratagene, La Jolla, Calif.) and purified with ProbeQuant™ G-50 Micro Columns (Amersham Pharmacia Biotech Inc.) are added and hybridized to the blots with shaking at 65° C. for overnight. The blots are washed in 2×SSC, 0.1% (w/v) SDS at room temperature for 20 minutes, twice in 1×SSC, 0.1% (w/v) SDS at 65° C. for 15 minutes, then exposed to Hyperfilms (Amersham Life Science).

### Example 6

### Analysis of Expression of Gene Corresponding to SK2 (Cluster 9083 (c9083)) (SEQ ID NO:3) in Colorectal Carcinoma

The expression of the gene comprising the sequence of SK2, which clusters to cluster i.d. no. 9083, was examined by quantitative PCR in several cancer cell lines, including a number of colorectal carcinoma cell lines. The cells in which expression was tested are summarized below.

| Cell Line | Tissue Source | Cell Line | Tissue Source |
|---|---|---|---|
| MDA-MB-231 | Human breast; high metastatic | Caco-2 | Human colorectal |
| | potential (micromets in lung; | | adenocarcinoma |
| | adenocarcinoma; pleural | | |
| | effusion | | |
| MDA-MB-435 | Human breast, high metastatic | SW620 | Human colorectal |
| | potential (macrometastases in | | adenocarcinoma; from |
| | lung) | | metastatic site (lymph node) |
| MCF-7 | Human breast; non-metastatic | LS174T | High metastatic potential |
| | | | human colorectal |
| | | | adenocarcinoma |
| MDA-MB-468 | Human breast; adenocarcinoma | LOVO | Human colorectal |
| | | | adenocarcinoma; colon; from |
| | | | metastatic site (colon) |
| Alab | Human breast, metastatic | HT29 | Human colorectal |
| | | | adenocarcinoma; colon |
| SKOV3 | Human ovarian | SW480 | Human colorectal |
| | adenocarcinoma | | adenocarcinoma; colon |
| OVCAR3 | Human ovarian | HCT116 | Human colorectal carcinoma; |
| | adenocarcinoma | | colon |
| KM12C | Human colon; low metastatic | Colo | Human colorectal |
| | potential | 320DN | adenocarcinoma; colon |
| KM12L4 | Human colon; high metastatic | T84 | Human colorectal carcinoma; |
| | potential (derived from | | colon; from metastatic site |
| | Km12C) | | (lung) |
| DU 145 | Human prostate; carcinoma; | HCT15 | Human colorectal |
| | from metastatic site: brain | | adenocarcinoma; colon |
| HT1080 | Human sarcoma cell line; | CCD112 | Human colorectal |
| | | | adenocarcinoma, low |
| | | | metastatic potential |
| HMVEC | Primary human microvascular | DLD1 | Human colon; colorectal |
| | endothelial cells | | adenocarcinoma |
| 185B4 | normal breast epithelial cells; | 293 | kidney epithelial cells |
| | chemically transformed | | |
| LNCAP | prostate carcinoma; metastasis | GRDP | primary prostate epithelium |
| | to left supraclavicular lymph | | |
| U373MG | glioblastoma cell | IMR90 | primary lung fibroblast |
| WOCA | primary prostate epithelium | PC3 | prostate cancer; androgen |
| | | | receptor negative |

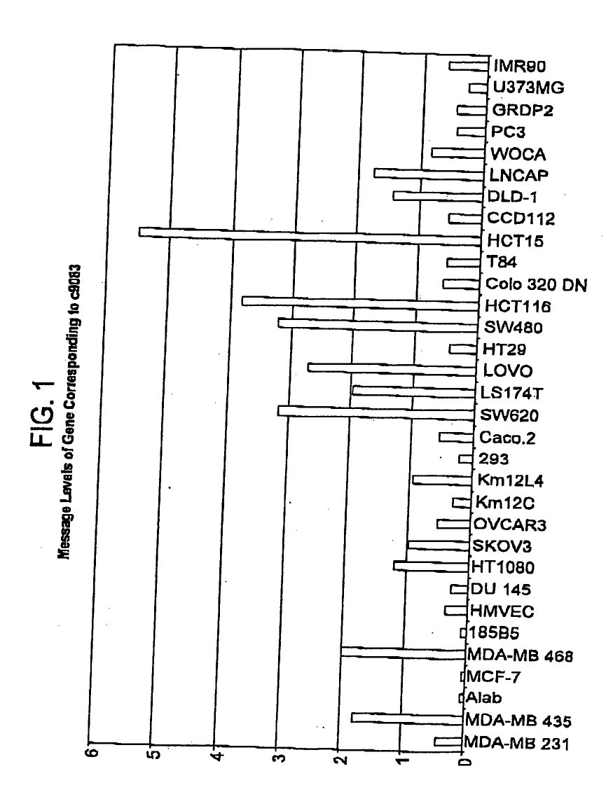

Quantitative real-time PCR was performed by first isolating RNA from cells using a Roche RNA Isolation kit according to manufacturer's directions. One microgram of RNA was used to synthesize a first-strand cDNA using MMLV reverse transcriptase (Ambion) using the manufacturers buffer and recommended concentrations of oligo dT, nucleotides, and Rnasin. This first-strand cDNA served as a template for quantitative real-time PCR using the Roche light-cycler as recommended in the machine manual. The gene corresponding to SK2 (C9083) (SEQ ID NO:3) was amplified with forward primer: 5′-cgctgacctcaaccag-3′ (SEQ ID NO:60) and reverse primer: 5′-ctgtttgcccgttcttattac-3′ (SEQ ID NO:61). Product was quantified based on the cycle at which the amplification entered the linear phase of amplification in comparison to an internal standard and using the software supplied by the manufacturer. Small differences in amounts or total template in the first-strand cDNA reaction were eliminated by normalizing to amount of actin amplified in a separate quantitative PCR reaction using the forward primer 5′-CGGGAAATCGTGCGTGACATTAAG-3′ (SEQ ID NO:56) and the reverse primer: 5′-TGATCTCCTTCTGCATCCTGTCGG-3′ (SEQ ID NO:57). The results are shown in FIG. 1

### Example 7

### Functional Analysis of Gene Corresponding to SK2 (c9083) (SEQ ID NO:3)

In order to further assess the role of the gene corresponding to SK2 (c9083) (SEQ ID NO:3), the functional information on the gene corresponding to this sequence was obtained using antisense knockout technology. In short, the cell type to be tested, SW620 or HT1080 cells which express the polypeptide encoded by the gene corresponding to c9083, were plated to approximately 60-80% confluency on 6-well or, for proliferation assays, 96-well dishes. Antisense or reverse control oligonucleotide was diluted to 2 μM in optimem and added to optimem into which the delivery vehicle, lipitoid 116-6 in the case of SW620 cells or 1:1 lipitoid 1:cholesteroid 1 in the case of HT1080 cells, had been diluted. The oligo/delivery vehicle mixture was then further diluted into medium with serum on the cells. The final concentration of oligonucleotide for all experiments was 300 nM, and the final ratio of oligo to delivery vehicle for all experiments was 1.5 nmol lipitoid/μg oligonucleotide. Cells were transfected overnight at 37 C and the transfection mixture was replaced with fresh medium the next morning.

The following antisense oligonucleotides were tested for the ability to deplete c9083 (SEQ ID NO:3) RNA:

| Olig Name | Sequence | Nucleotides |
|---|---|---|
| CHIR-8-4AS | ATTTGGGCATCACTGGCTACAAGCA | 25 |
| C9083:P0463 | (SEQ ID NO:64) | |
| CHIR-8-4RC | ACGAACATCGGTCACTACGGGTTTA | 25 |
| C9083:P0463RC | (SEQ ID NO:65) | |
| CHIR-8-5A5 | CAGAGAGGTGAGACACTCGCCGCA | 24 |
| C9083:P0157 | (SEQ ID NO:66) | |
| CHIR-8-5RC | ACGCCGCTCACAGAGTGGAGAGAC | 24 |
| C9083:POI57RC | (SEQ ID NO:67) | |

RC: reverse control oligos (control oligos);
AS: antisense oligos (test)

The effect of the oligonucleotide on the cells was assessed by both quantitation of PCR levels as described above, and in proliferation assays using amount of DNA as quantified with the Stratagene Quantos™ kit to determine cell number.

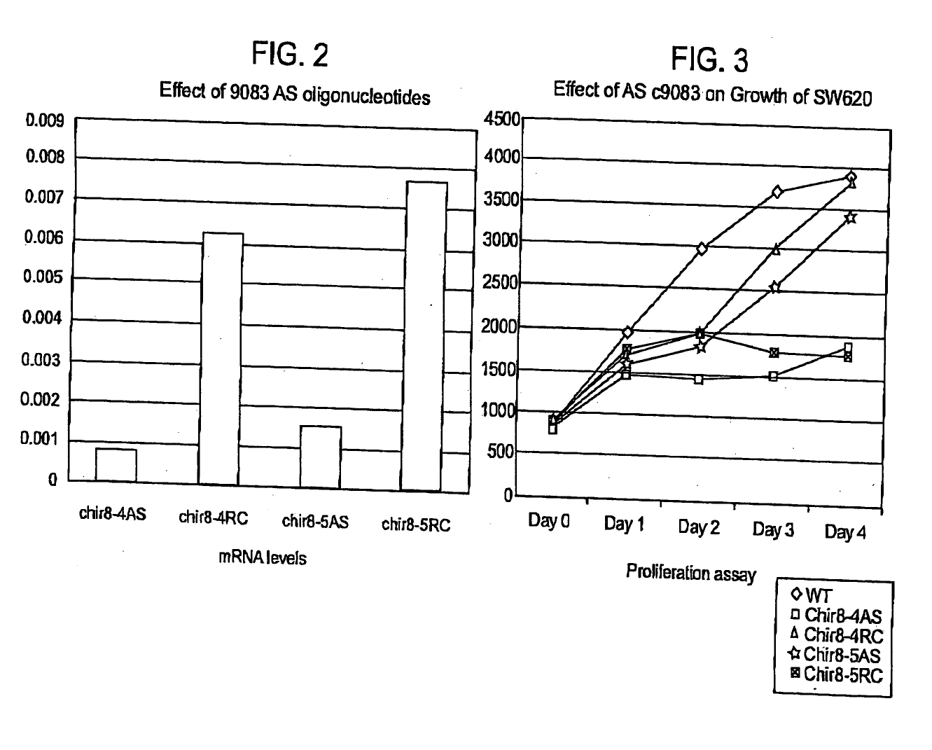

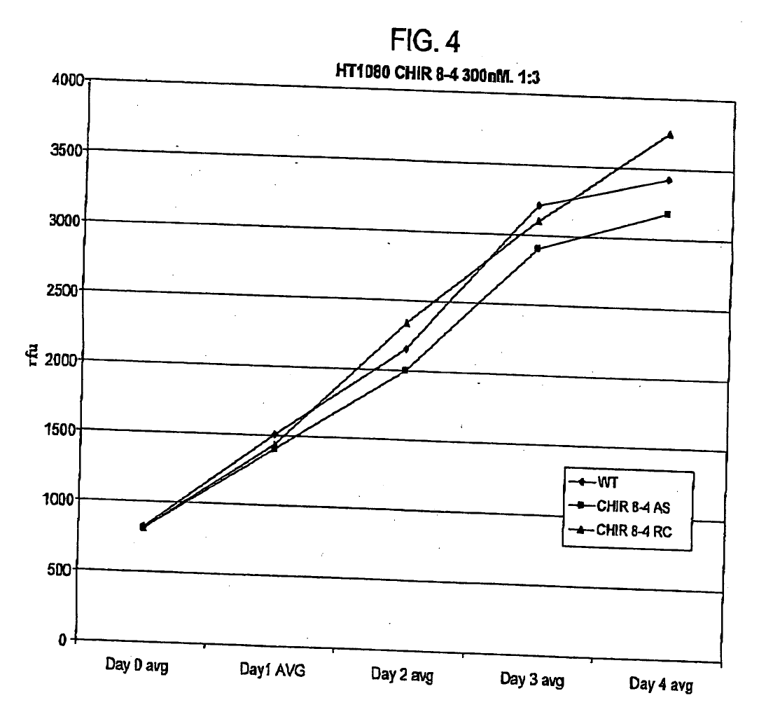

The results of the mRNA level quantitation are shown in FIG. 2. The effects of the oligonucleotides upon proliferation over a four day period are shown in FIGS. 3 and 4. Cells without oligonucleotide treatment (WT) served as a control. The oligo CHIR-8-4AS was most effective in decreasing mRNA for the gene corresponding to 9083c. Transfection of these oligos into SW620 cells resulted in a decreased rate of proliferation relative to matched reverse control oligos, with CHIR-8-4 being somewhat more effective than CHIR-8-5 (FIG. 3). Significantly, the same antisense oligonucleotide had no effect on growth of a fibrosarcoma cell line, HT1080 (FIG. 4). This indicates that the functional role of the gene corresponding to c9083 is tissue-specific, and further that the gene corresponding to c9083 has a specific effect on growth.

The oligos were next tested for their effect on colony formation in a soft agar assay. Soft agar assays were conducted by first establishing a bottom layer of 2 ml of 0.6% agar in media plated fresh within a few hours of layering on the cells. The cell layer was formed on the bottom layer by removing cells transfected as described above (either an antisense k-Ras oligo as a positive control), CHIR-8-4, CHIR-8-5, CHIR-8-4RC, or CHIR-8-5RC) from plates using 0.05% trypsin and washing twice in media. The cells were counted in a Coulter counter, and resuspended to 10⁶ per ml in media. 10 μl aliquots are placed with media in 96-well plates (to check counting with WST1), or diluted further for soft agar assay. 2000 cells are plated in 800 μl 0.4% agar in duplicate wells above 0.6% agar bottom layer. After the cell layer agar solidifies, 2 ml of media is dribbled on top and antisense or reverse control oligo is added without delivery vehicles. Fresh media and oligos are added every 3-4 days. Colonies are formed in 10 days to 3 weeks. Fields of colonies were counted by eye. WST-1 metabolism values can be used to compensate for small differences in starting cell number. Larger fields can be scanned for visual record of differences.

Both the CHIR-8-4 and CHIR-8-5 antisense oligos led to decreased colony size and number compared to the control CHIR-8-4RC and CHIR-8-5RC oligos. These results further validate the gene corresponding to c9083 (SEQ ID NO:3) as a target for therapeutic intervention.

### Example 8

### Effect of Antisense Oligonucleotides on Message Levels for Target Genes

The effect of antisense oligonucleotides upon message levels for the genes corresponding to the sequences and clusters described herein was analyzed using antisense knockout technology as described for c9083 in the Example above. Specifically, antisense oligos for genes corresponding to each of c719, c1665, c3376, c115762, c454001, c3788805, and c776682 were prepared as described above. Once synthesized and quantitated, the oligomers were screened for efficiency of a transcript knock-out in a panel of cancer cell lines. The efficiency of the knock-out was determined by analyzing mRNA levels using lightcycler quantification. The oligomers that resulted in the highest level of transcript knock-out, wherein the level was at least about 50%, preferably about 80-90%, up to 95% or more up to undetectable message, were selected for use in a cell-based proliferation assay, an anchorage independent growth assay, and an apoptosis assay.

SW620 cells, which express the polypeptide encoded by the corresponding genes to be analyzed, were plated to approximately 60-80% confluency on 6-well or, for proliferation assays, 96-well dishes. For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense or control oligonucleotide was then prepared to a working concentration of 100 μM in sterile Millipore water. The oligonucleotide was further diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 mmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down. Oligonucleotide was added to the cells to a final concentration of 30 nM.

The level of target mRNA that corresponds to a target gene of interest in the transfected cells was quantitated in the cancer cell lines using the Roche LightCycler™ real-time PCR machine. Values for the target mRNA were normalized versus an internal control (e.g., beta-actin). For each 20 μl reaction, extracted RNA (generally 0.2-1 μg total) was placed into a sterile 0.5 or 1.5 ml microcentrifuge tube, and water was added to a total volume of 12.5 μl. To each tube was added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin® (20 u) (Ambion, Inc., Hialeah, Fla.), and 0.5 μl MMLV reverse transcriptase (50 u) (Ambion, Inc.). The contents were mixed by pipetting up and down, and the reaction mixture was incubated at 42° C. for 1 hour. The contents of each tube were centrifuged prior to amplification.

An amplification mixture was prepared by mixing in the following order: 1×PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10× concentration from Perkin-Elmer, Norwalk, Conn.). In 1× concentration it contains 10 mM Tris pH 8.3 and 50 mM KCl. SYBR® Green (Molecular Probes, Eugene, Oreg.) is a dye which fluoresces when bound to double stranded DNA. As double stranded PCR product is produced during amplification, the fluorescence from SYBR® Green increases. To each 20 μl aliquot of amplification mixture, 2 μl of template RT was added, and amplification was carried out according to standard protocols.

The following antisense oligonucleotides were tested for the ability to deplete the message levels of the gene corresponding to the indicated cluster. Target Gene: Oligo Location provides the name of the cluster to which the target gene is assigned and the name of the oligo used. AS indicates antisense; RC indicates reverse control. Data for the genes corresponding to c9083 are provided for comparison.

| Target | | | % KO of |
|---|---|---|---|
| Gene:Oligo Location | Oligo Sequence | SEQ ID NO: | Message |
| c719:1-AS | TTGGTGTCATTGGGTCAAGGGTTGG | 68 | 85% |
| C719:1-RC | GGTTGGGAACTGGGTTACTGTGGTT | 69 | |
| c719:2-AS | ACAGGGCAGATACGGACCTCGGTG | 70 | 93% |
| c719:2-RC | GTGGCTCCAGGCATAGACGGGACA | 71 | |
| c719:3-AS | TTGTGGGTAAGCAGTTTCATGTCGC | 72 | 67% |
| c719:3-RC | CGCTGTACTTTGACGAATGGGTGTT | 73 | |
| c719:4-AS | CCTGGATCAGACGCAAGTTATCGGC | 74 | 85% |
| c719:4-RC | CGGCTATTGAACGCAGACTAGGTCC | 75 | |
| C9083:4-AS | ATTTGGGCATCACTGGCTACAAGCA | 64 | 83.0 |
| C9083:4-RC | ACGAACATCGGTCACTACGGGTTTA | 65 | |
| C9083:5-AS | CAGAGAGGTGAGACACTCGCCGCA | 66 | 73.0 |
| C9083:5-RC | ACGCCGCTCACAGAGTGGAGAGAC | 67 | |
| C1665:1-AS | CTACTCCCCACACTTCATCGCCAGG | 76 | 73.0 |
| C1665:1-RC | GGACCGCTACTTCACACCCCTCATC | 77 | |
| C1665:2-AS | CTCTTGATACTCCAGCGGCAAACCA | 78 | 81.0 |
| C1665:2-RC | ACCAAACGGCGACCTCATAGTTCTC | 79 | |
| c3376:1-AS | GCGCCCAAGCCGTTCGTTCTTAAG | 80 | 78.0 |
| c3376:1-RC | GAATTCTTGCTTGCCGAACCCGCG | 81 | |
| c3376:2-AS | CCAGGTAGGCACGAGTTGGCAAAGA | 82 | 97.0 |
| c3376:2-RC | AGAAACGGTTGAGCACGGATGGACC | 83 | |
| c3376:3-AS | GCCATTGAAGATGCCCAGATCCCAC | 84 | 56.0 |
| c3376:3-RC | CACCCTAGACCCGTAGAAGTTACCG | 85 | |
| c3376:4-AS | CCTGCGTTTGTCCCTCCAGCATCT | 86 | 93.0 |
| c3376:4-RC | TCTACGACCTCCCTGTTTGCGTCC | 87 | |
| c3376:5-AS | AAGTCACAGTCCCCGGATACCAGTC | 88 | 88.0 |
| c3376:5-RC | CTGACCATAGGCCCCTGACACTGAA | 89 | |
| c115762:1-AS | TTGTCGCTTTGGCAGGCATAAAACC | 90 | 97.5 |
| c115762:2-AS | TCTGGTCATCAACTTGCTTTCCGTG | 91 | 99.0 |
| c115762:3-AS | CAGTGTTTCGTGGTGTGCTCTGTGG | 92 | 98.0 |
| c115762:4-AS | GCTCACCATCCGGGCACCAAGCA | 93 | 97.0 |
| c115762:5-AS | TGAGAGACAGTGTTTCGTGGTGTGC | 94 | 93.0 |
| 454001:1-AS | TGCCTTCACACGCTTGGTTATCTTC | 95 | 0 |
| 454001:2-AS | GACAACATCGGAGGCTTCAATCACC | 96 | 0 |
| 454001:3-AS | GTTGAGGCTCTGAACACCACTGTTG | 97 | 0 |
| 454001:4-AS | GTTTGGCAGCACCTTCAACATTTGG | 98 | 87 |
| 454001:5-AS | AGCAGTTTGGCAGCACCTTCAACA | 99 | 92 |
| 454001:-1-RC | CTTCTATTGGTTCGCACACTTCCGT | 100 | |
| 454001:2-RC | CCACTAACTTCGGAGGCTACAACAG | 101 | |
| 454001:3-RC | GTTGTCACCACAAGTCTCGGAGTTG | 102 | |
| 454001:4-RC | GGTTTACAACTTCCACGACGGTTTG | 103 | |
| 454001:5-RC | ACAACTTCCACGACGGTTTGACGA | 104 | |
| 378805:1-AS | ATCTGGCATGGACGGATGAGCGAA | 105 | 41.0 |
| 378805:2-AS | GCTGGGTGGTTTCCGAACTCAACG | 106 | 97 |
| 378805:3-AS | GTCCCAATCACCTTCCCCACAATCC | 107 | 65.0 |
| 378805:4-AS | TCAGATCCTTCTTCCACTCCCGCTT | 108 | 100.0 |
| 378805:5-AS | TGCTCGTGGAACAGGTAAAGCTCTG | 109 | 98 |
| 378805:1-RC | AAGCGAGTAGGCAGGTACGGTCTA | 110 | |
| 378805:2-RC | GCAACTCAAGCCTTTGGTGGGTCG | 111 | |
| 378805:3-RC | CCTAACACCCCTTCCACTAACCCTG | 112 | |
| 378805:4-RC | TTCGCCCTCACCTTCTTCCTAGACT | 113 | |
| 378805:5-RC | GTCTCGAAATGGACAAGGTGCTCGT | 114 | |
| 776682:1-AS | AGCTTCACTTTGGTCTTGACGGCAT | 115 | 81 |
| 776682:2-AS | CGGAGGGAAGTCAAGTCAGCCACA | 116 | 60 |
| 776682:3-AS | CGGCATTCACCCTCTCCAGCACCT | 117 | 89 |
| 776682:4-AS | CCTCCACCTGTTTGCGGGCTTCC | 118 | 61 |
| 776682:5-AS | CCACATTGAGGGAGTCCTCTTGCAA | 119 | 80 |
| 776682:1-RC | TACGGCAGTTCTGGTTTCACTTCGA | 120 | |
| 776682:2-RC | ACACCGACTGAACTGAAGGGAGGC | 121 | |
| 776682:3-RC | TCCACGACCTCTCCCACTTACGGC | 122 | |
| 776682:5-RC | CCTTCGGGCGTTTGTCCACCTCC | 123 | |
| 402380:P464:4-AS | CCCCGAACAAAACACCAGTCAACG | 124 | 94 |
| 402380:P464:4-RC | GCAACTGACCACAAAACAAGCCCC | 125 | |
| 402380:P414:5 AS | GGCCATTGAGTCCCTCCATAGCAGC | 126 | 92 |
| 402380:P414:5-RC | CGACGATACCTCCCTGAGTTACCGG | 127 | |

The effect of the oligonucleotide on the cells was assessed by quantitation of PCR levels. The results of the mRNA level quantitation are summarized in the table immediately above.

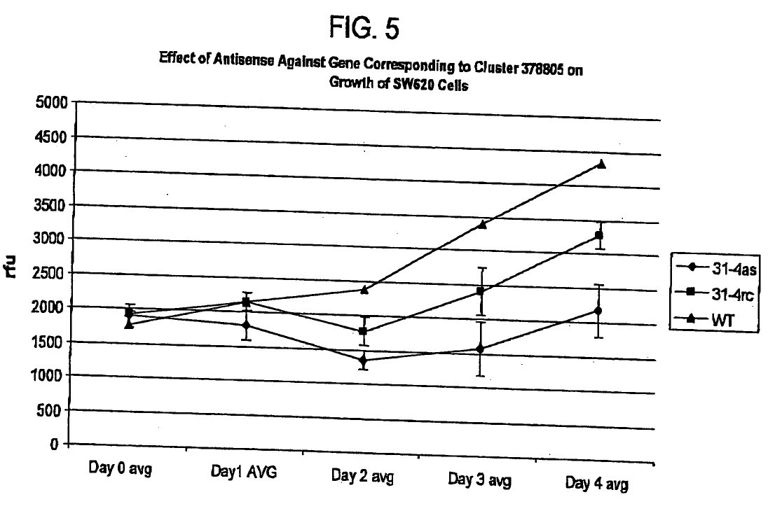

The effect of the loss of message for each gene above can be assessed in cell-based assays as described in Example 7 above. One such use of the antisense oligonucleotide described by SEQ ID NO:108 resulted in an inhibition of proliferation of SW620 cells when used as described in the transfection and proliferation assay protocols in Example 7 (FIG. 5).

### Example 9

### The Effect of Expression of Genes Corresponding to c3376 and 402380 Upon on Proliferation

The effect of expression of genes corresponding to c3376 (gene corresponding to SEQ ID NO:13) and 402380 (gene corresponding to SEQ ID NO:16) on the inhibition of cell proliferation was assessed in SW620 colon colorectal carcinoma cells.

Cells were plated to approximately 60-80% confluency in 96-well dishes. Antisense or reverse control oligonucleotide was diluted to 2 μM in OptiMEM™ and added to OptiMEM™ into which the delivery vehicle, lipitoid 116-6 in the case of SW620 cells or 1:1 lipitoid 1:cholesteroid 1 in the case of MDA-MB-231 cells, had been diluted. The oligo/delivery vehicle mixture was then further diluted into medium with serum on the cells. The final concentration of oligonucleotide for all experiments was 300 nM, and the final ratio of oligo to delivery vehicle for all experiments was 1.5 nmol lipitoid/μg oligonucleotide.

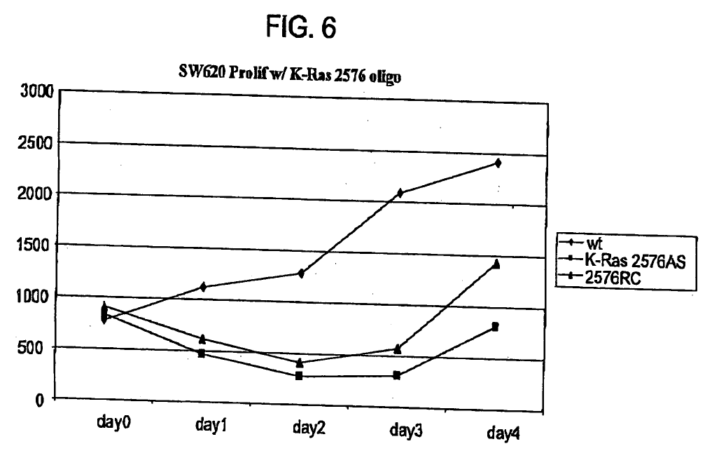

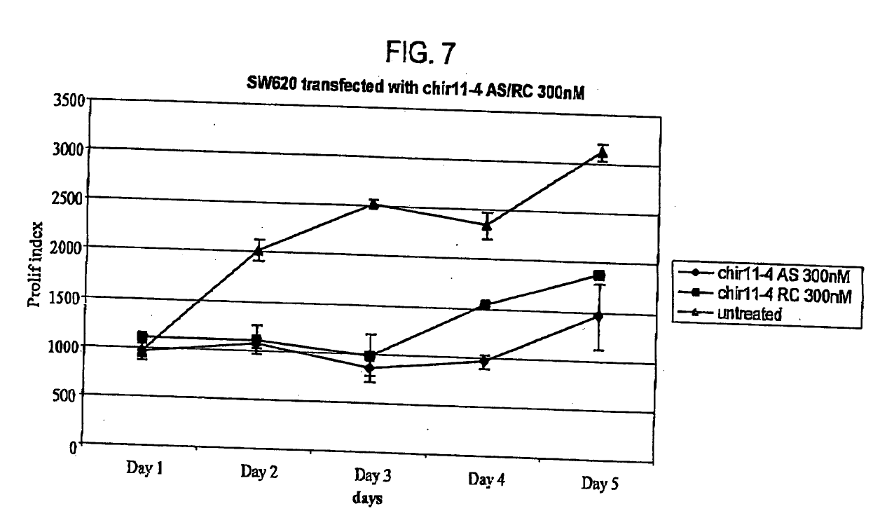

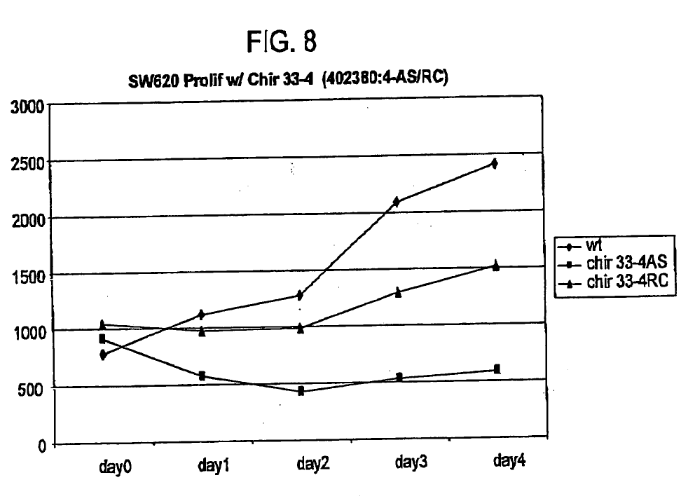

Antisense oligonucleotides were prepared as described above. Cells were transfected overnight at 37° C. and the transfection mixture was replaced with fresh medium the next morning. Transfection was carried out as described above in Example 8. Proliferaton was measured using the colormetric reagent WST-1 according to methods well known in the art. The results of the antisense experiments are shown in FIGS. 6-9. The values on the y-axis represent relative fluorescent units. Antisense and reverse control oligos to K-Ras served as a control to demonstrate the assay worked as expected (FIG. 6).

### Example 10

### Effect of Gene Expression on Colony Formation in Soft Agar

The effect of expression of the gene corresponding to 402380 (gene corresponding to SEQ ID NO:16) upon colony formation of SW620 cells was tested in a soft agar assay. Soft agar assays were conducted by first establishing a bottom layer of 2 ml of 0.6% agar in media plated fresh within a few hours of layering on the cells. The cell layer was formed on the bottom layer by removing cells transfected as described above from plates using 0.05% trypsin and washing twice in media. The cells were counted in a Coulter counter, and resuspended to 10⁶ per ml in media. 10 μl aliquots were placed with media in 96-well plates (to check counting with WST-1), or diluted further for the soft agar assay. 2000 cells were plated in 800 μl 0.4% agar in duplicate wells above 0.6% agar bottom layer. After the cell layer agar solidified, 2 ml of media was dribbled on top and antisense or reverse control oligo (produced as described above) was added without delivery vehicles. Fresh media and oligos were added every 3-4 days. Colonies formed in 10 days to 3 weeks. Fields of colonies were counted by eye. Wst-1 metabolism values were used to compensate for small differences in starting cell number. Larger fields can be scanned for visual record of differences.

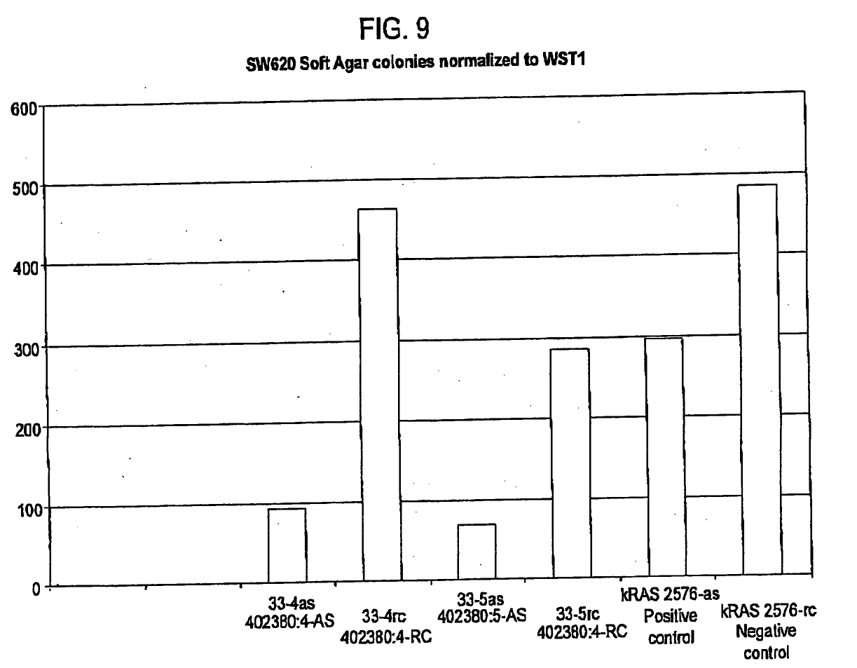

The results are shown in FIG. 9. The y-axis represents the number of cells per a defined sector, using WST-1 to facilitate cell count and normalized to a control. Antisense and reverse control oligos to K-Ras (kRAS 2576-as and kRAS 2576-rc) served as controls to demonstrate the assay worked as expected.

### Example 11

### Effect of Gene Expression Upon Cell Death

Effect of expression of the genes corresponding to cluster 719 (gene corresponding to SEQ ID NO:1, CHIR-7); cluster 9083 (gene corresponding to SEQ ID NO:3, CHIR-8); cluster 1665 (gene corresponding to SEQ ID NOS:7 and 9, CHIR-9); cluster 3376 (gene corresponding to SEQ ID NO:13, CHIR-11); cluster 115762 (gene corresponding to SEQ ID NO:5, CHIR-16); and cluster 402380 (gene corresponding to SEQ ID NO:16, CHIR-33) upon cell death in an lactatae dehydrobenase (LDH) cytotoxitity assay was examined in HT1080 cells (a human fibrosarcoma cell line), SW620 cells, and metastatic breast cancer cell lines (MDA-MB-231 (“231”)) cells. The lactate dehydrogenase (LDH) cytotoxicity assay essentially as follows:

The lactate dehydrogenase (LDH) cytotoxicity assay was performed essentially as follows:

Day 1: Cells were seeded in 4 separate 96 well plates, typically 5000 cells/well and incubated at 37° C. and 5% CO₂.

Day 2: Cells were transfected with the anti-sense as well as the reverse complement controls, essentially as described in Example 4. One plate (day 0) was left untransfected as a seeding control.

The transfection was carried out using a lipid vehicle for delivery as described in WO 01/16306, hereby incorporated in its entirety. Briefly, the transfection used agents known as “lipitoids” and “cholesteroids”, described, for example, in PCT publications WO 01/16306, WO 98/06437 and WO 99/08711, based on U.S. Ser. Nos. 60/023,867, 60/054,743, and 09/132,808, which are also hereby incorporated by reference. These lipid-cationic peptoid conjugates are shown in these references to be effective reagents for the delivery of plasmid DNA to cells in vitro. Any of the carriers described in the above-referenced applications are suitable for use in transfection of the oligonucleotides described herein.

These compounds may be prepared by conventional solution or solid-phase synthesis. In one such procedure, as described in WO 99/08711, cited above, the N-terminus of a resin-bound peptoid is acylated with a spacer such as Fmocaminohexanoic acid or Fmoc-3-alanine. After removal of the Fmoc group, the primary amino group is reacted with cholesterol chloroformate to form a carbamate linkage. The product is then cleaved from the resin with trifluoroacetic acid and purified by reverse-phase HPLC. A fatty acid-derived lipid moiety, such as a phospholipid, may be used in place of the steroid moiety. The steroid or other lipid moiety may also be linked to the peptoid moiety by other linkages, of any effective length, readily available to the skilled practitioner.

Depending on the cell type, different lipid vehicles were used for different lengths of time for transfection. However, the transfection time did not exceed 24 hrs. The transfection was carried out in complete medium and the final anti-sense oligonucleotide concentration was 300 nM per well. In the wells with drug, the drug was added to the culture at the beginning of the transfection.

Starting on day 3: cells were recovered, 1 plate/day and release of LDH into the supernatant as well as LDH in intact cells was measured using a kit from Roche according to manufacturer's instructions (Roche Diagnostics, Basel, Switzerland) (data labeled as day 1, 2, 3).

For each sample, were analyzed by examining the relative level of released LDH compared to total LDH, wherein an increase as a portion of total LDH signifies increased cell death (due to a higher proportion of released LDH in the media). The data was assessed qualitatively by comparison to an untreated control (no oligo). This assay allowed a determination as to whether antisense-induced loss of message for a particular gene causes death of cells when used alone, or wheter this loss of message sensitizes cells to the effects of a drug.

The results are shown in the table immediately below.

| | HT1080 | SW620 | 231 |
|---|---|---|---|
| chir7-2 | negative | negative | |
| chir8-4 | positive | weakly positive | |
| chir9-5 | | positive | |
| chir11-2 | | negative | |
| chir16-4 | | negative | |
| chir33-4 | very weakly | strong positive | very weakly |
| | positive | | positive |

### Example 12

### Detection of Differential Expression Using Arrays

mRNA isolated from samples of cancerous and normal colon tissue obtained from patients were analyzed to identify genes differentially expressed in cancerous and normal cells. Normal and cancerous cells collected from cryopreserved patient tissues were isolated using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001).

Table 5 (inserted before the claims) provides information about each patient from which the samples were isolated, including: the “Patient ID” and “Path ReportID”, which are numbers assigned to the patient and the pathology reports for identification purposes; the “Group” to which the patients have been assigned; the anatomical location of the tumor (“Anatom Loc”); the “Primary Tumor Size”; the “Primary Tumor Grade”; the identification of the histopathological grade (“Histopath Grade”); a description of local sites to which the tumor had invaded (“Local Invasion”); the presence of lymph node metastases (“Lymph Node Met”); the incidence of lymph node metastases (provided as a number of lymph nodes positive for metastasis over the number of lymph nodes examined) (“Incidence Lymphnode Met”); the “Regional Lymphnode Grade”; the identification or detection of metastases to sites distant to the tumor and their location (“Distant Met & Loc”); a description of the distant metastases (“Descrip Distant Met”); the grade of distant metastasis (“Dist Met Grade”); and general comments about the patient or the tumor (“Comments”). Adenoma was not described in any of the patients; adenoma dysplasia (described as hyperplasia by the pathologist) was described in Patient ID No. 695. Extranodal extensions were described in two patients, Patient ID Nos. 784 and 791. Lymphovascular invasion was described in seven patients, Patient ID Nos. 128, 278, 517, 534, 784, 786, and 791. Crohn's-like infiltrates were described in seven patients, Patient ID Nos. 52, 264, 268, 392, 393, 784, and 791.

TABLE 5

| | | | | Primary | Primary | | | | Incidence | Regional Lymp | Distant | Descrip | | |
|---|---|---|---|---|---|---|---|---|---|---|---|---|---|---|
| Patient | Path Report | | Anatom | Tumor | Tumor | Histo path | Local | Lymph node | Lymph | node | Met & | Distant | Dist Met | |
| ID | ID | Group | Loc | Size | Grade | Grade | Invasion | Met | node Met | Grade | Loc | Met | Grade | Comment |
| 15 | 21 | III | Ascending | 4.0 | T3 | G2 | extending | positive | 3/8 | N1 | negative | | MX | invasive |
| | | | colon | | | | into | | | | | | | adenocarcinoma, |
| | | | | | | | subserosal | | | | | | | moderately |
| | | | | | | | adipose | | | | | | | differentiated; |
| | | | | | | | tissue | | | | | | | focal |
| | | | | | | | | | | | | | | perineural |
| | | | | | | | | | | | | | | invasion is |
| | | | | | | | | | | | | | | seen |
| 52 | 71 | II | Ascending | 9.0 | T3 | G3 | Invasion | negative | 0/12 | N0 | negative | | M0 | Hyperplastic |
| | | | colon | | | | through | | | | | | | polypin |
| | | | | | | | muscularis | | | | | | | appendix. |
| | | | | | | | propria, | | | | | | | |
| | | | | | | | subserosal | | | | | | | |
| | | | | | | | involvement; | | | | | | | |
| | | | | | | | ileocec. | | | | | | | |
| | | | | | | | valve | | | | | | | |
| | | | | | | | involvement | | | | | | | |
| 121 | 140 | II | Sigmoid | 6 | T4 | G2 | Invasion | negative | 0/34 | N0 | negative | | M0 | Perineural |
| | | | | | | | of | | | | | | | Invasion; |
| | | | | | | | muscularis | | | | | | | donut |
| | | | | | | | propria | | | | | | | anastomos |
| | | | | | | | into | | | | | | | is |
| | | | | | | | serosa, | | | | | | | negative. |
| | | | | | | | involving | | | | | | | One |
| | | | | | | | submucosa | | | | | | | tubulovillous |
| | | | | | | | of | | | | | | | and |
| | | | | | | | urinary | | | | | | | one |
| | | | | | | | bladder | | | | | | | tubular |
| | | | | | | | | | | | | | | adenoma |
| | | | | | | | | | | | | | | with no |
| | | | | | | | | | | | | | | high grade |
| | | | | | | | | | | | | | | dysplasia. |
| 125 | 144 | II | Cecum | 6 | T3 | G2 | Invasion | negative | 0/19 | N0 | negative | | M0 | patient |
| | | | | | | | through | | | | | | | history of |
| | | | | | | | the | | | | | | | metastatic |
| | | | | | | | muscularis | | | | | | | melanoma |
| | | | | | | | propria | | | | | | | |
| | | | | | | | into | | | | | | | |
| | | | | | | | suserosal | | | | | | | |
| | | | | | | | adipose | | | | | | | |
| | | | | | | | tissue. | | | | | | | |
| | | | | | | | Ileocecal | | | | | | | |
| | | | | | | | junction. | | | | | | | |
| 128 | 147 | III | Transverse | 5.0 | T3 | G2 | Invasion | positive | 1/5 | N1 | negative | | M0 | |
| | | | colon | | | | of | | | | | | | |
| | | | | | | | muscularis | | | | | | | |
| | | | | | | | propria | | | | | | | |
| | | | | | | | into | | | | | | | |
| | | | | | | | percolonic | | | | | | | |
| | | | | | | | fat | | | | | | | |
| 130 | 149 | | Splenic | 5.5 | T3 | | through | positive | 10/24 | N2 | negative | | M1 | |
| | | | flexure | | | | wall | | | | | | | |
| | | | | | | | and | | | | | | | |
| | | | | | | | into | | | | | | | |
| | | | | | | | surrounding | | | | | | | |
| | | | | | | | adipose | | | | | | | |
| | | | | | | | tissue | | | | | | | |
| 133 | 152 | II | Rectum | 5.0 | T3 | G2 | Invasion | negative | 0/9 | N0 | negative | | M0 | Small |
| | | | | | | | through | | | | | | | separate |
| | | | | | | | muscularis | | | | | | | tubular |
| | | | | | | | propria | | | | | | | adenoma |
| | | | | | | | into | | | | | | | (0.4 cm) |
| | | | | | | | non- | | | | | | | |
| | | | | | | | peritonealized | | | | | | | |
| | | | | | | | pericolic | | | | | | | |
| | | | | | | | tissue; | | | | | | | |
| | | | | | | | gross | | | | | | | |
| | | | | | | | configuration | | | | | | | |
| | | | | | | | is | | | | | | | |
| | | | | | | | annular. | | | | | | | |
| 141 | 160 | IV | Cecum | 5.5 | T3 | G2 | Invasion | positive | 7/21 | N2 | positive | adenocarcinoma | M1 | Perineural |
| | | | | | | | of | | | | (Liver) | consistant | | invasion |
| | | | | | | | muscularis | | | | | with | | identified |
| | | | | | | | propria | | | | | primary | | adjacent |
| | | | | | | | into | | | | | | | to |
| | | | | | | | pericolonic | | | | | | | metastatic |
| | | | | | | | adipose | | | | | | | adenocarcinoma. |
| | | | | | | | tissue, | | | | | | | |
| | | | | | | | but | | | | | | | |
| | | | | | | | not | | | | | | | |
| | | | | | | | through | | | | | | | |
| | | | | | | | serosa. | | | | | | | |
| | | | | | | | Arising | | | | | | | |
| | | | | | | | from | | | | | | | |
| | | | | | | | tubular | | | | | | | |
| | | | | | | | adenoma. | | | | | | | |
| 156 | 175 | III | Hepatic | 3.8 | T3 | G2 | Invasion | positive | 2/13 | N1 | negative | | M0 | Separate |
| | | | flexure | | | | through | | | | | | | tubolovillous |
| | | | | | | | mucsularis | | | | | | | and |
| | | | | | | | propria | | | | | | | tubular |
| | | | | | | | into | | | | | | | adenomas |
| | | | | | | | subserosa/ | | | | | | | |
| | | | | | | | pericolic | | | | | | | |
| | | | | | | | adipose, | | | | | | | |
| | | | | | | | no | | | | | | | |
| | | | | | | | serosal | | | | | | | |
| | | | | | | | involvement. | | | | | | | |
| | | | | | | | Gross | | | | | | | |
| | | | | | | | configuration | | | | | | | |
| | | | | | | | annular. | | | | | | | |
| 228 | 247 | III | Rectum | 5.8 | T3 | G2 to | Invasion | positive | 1/8 | N1 | negative | | MX | Hyperplastic |
| | | | | | | G3 | through | | | | | | | polyps |
| | | | | | | | muscularis | | | | | | | |
| | | | | | | | propria | | | | | | | |
| | | | | | | | to | | | | | | | |
| | | | | | | | involve | | | | | | | |
| | | | | | | | subserosal, | | | | | | | |
| | | | | | | | perirectoal | | | | | | | |
| | | | | | | | adipose, | | | | | | | |
| | | | | | | | and | | | | | | | |
| | | | | | | | serosa | | | | | | | |
| 264 | 283 | II | Ascending | 5.5 | T3 | G2 | Invasion | negative | 0/10 | N0 | negative | | M0 | Tubulovillous |
| | | | colon | | | | through | | | | | | | adenoma |
| | | | | | | | muscularis | | | | | | | with high |
| | | | | | | | propria | | | | | | | grade |
| | | | | | | | into | | | | | | | dysplasia |
| | | | | | | | subserosal | | | | | | | |
| | | | | | | | adipose | | | | | | | |
| | | | | | | | tissue. | | | | | | | |
| 266 | 285 | III | Transverse | 9 | T3 | G2 | Invades | negative | 0/15 | N1 | positive | 0.4 cm, | MX | |
| | | | colon | | | | through | | | | (Mesenteric | may | | |
| | | | | | | | muscularis | | | | deposit) | represent | | |
| | | | | | | | propria | | | | | lymph | | |
| | | | | | | | to | | | | | node | | |
| | | | | | | | involve | | | | | completely | | |
| | | | | | | | pericolonic | | | | | replaced | | |
| | | | | | | | adipose, | | | | | by tumor | | |
| | | | | | | | extends | | | | | | | |
| | | | | | | | to | | | | | | | |
| | | | | | | | serosa | | | | | | | |
| 268 | 287 | I | Cecum | 6.5 | T2 | G2 | Invades | negative | 0/12 | N0 | negative | | M0 | |
| | | | | | | | full | | | | | | | |
| | | | | | | | thickness | | | | | | | |
| | | | | | | | of | | | | | | | |
| | | | | | | | muscularis | | | | | | | |
| | | | | | | | propria, | | | | | | | |
| | | | | | | | but | | | | | | | |
| | | | | | | | mesenteric | | | | | | | |
| | | | | | | | adipose | | | | | | | |
| | | | | | | | free | | | | | | | |
| | | | | | | | of | | | | | | | |
| | | | | | | | malignancy | | | | | | | |
| 278 | 297 | III | Rectum | 4 | T3 | G2 | Invasion | positive | 7/10 | N2 | negative | | M0 | Descending |
| | | | | | | | into | | | | | | | colon |
| | | | | | | | perirectal | | | | | | | polyps, no |
| | | | | | | | adipose | | | | | | | HGD or |
| | | | | | | | tissue. | | | | | | | carcinoma |
| | | | | | | | | | | | | | | identified. |
| 295 | 314 | II | Ascending | 5.0 | T3 | G2 | Invasion | negative | 0/12 | N0 | negative | | M0 | Melanosis |
| | | | colon | | | | through | | | | | | | coli and |
| | | | | | | | muscularis | | | | | | | diverticular |
| | | | | | | | propria | | | | | | | disease. |
| | | | | | | | into | | | | | | | |
| | | | | | | | percolic | | | | | | | |
| | | | | | | | adipose | | | | | | | |
| | | | | | | | tissue. | | | | | | | |
| 339 | 358 | II | Rectosigmoid | 6 | T3 | G2 | Extends | negative | 0/6 | N0 | negative | | M0 | 1 |
| | | | | | | | into | | | | | | | hyperplastic |
| | | | | | | | perirectal | | | | | | | polyp |
| | | | | | | | fat | | | | | | | identified |
| | | | | | | | but | | | | | | | |
| | | | | | | | does | | | | | | | |
| | | | | | | | not | | | | | | | |
| | | | | | | | reach | | | | | | | |
| | | | | | | | serosa | | | | | | | |
| 341 | 360 | II | Ascending | 2 cm | T3 | G2 | Invasion | negative | 0/4 | N0 | negative | | MX | |
| | | | colon | invasive | | | through | | | | | | | |
| | | | | | | | muscularis | | | | | | | |
| | | | | | | | propria | | | | | | | |
| | | | | | | | to | | | | | | | |
| | | | | | | | involve | | | | | | | |
| | | | | | | | pericolonic | | | | | | | |
| | | | | | | | fat. | | | | | | | |
| | | | | | | | Arising | | | | | | | |
| | | | | | | | from | | | | | | | |
| | | | | | | | villous | | | | | | | |
| | | | | | | | adenoma. | | | | | | | |
| 356 | 375 | II | Sigmoid | 6.5 | T3 | G2 | Through | negative | 0/4 | N0 | negative | | M0 | |
| | | | | | | | colon | | | | | | | |
| | | | | | | | wall | | | | | | | |
| | | | | | | | into | | | | | | | |
| | | | | | | | subserosal | | | | | | | |
| | | | | | | | adipose | | | | | | | |
| | | | | | | | tissue. | | | | | | | |
| | | | | | | | No | | | | | | | |
| | | | | | | | serosal | | | | | | | |
| | | | | | | | spread | | | | | | | |
| | | | | | | | seen. | | | | | | | |
| 360 | 412 | III | Ascending | 4.3 | T3 | G2 | Invasion | positive | 1/5 | N1 | negative | | M0 | Two |
| | | | colon | | | | thru | | | | | | | mucosal |
| | | | | | | | muscularis | | | | | | | polyps |
| | | | | | | | propria | | | | | | | |
| | | | | | | | to | | | | | | | |
| | | | | | | | pericolonic | | | | | | | |
| | | | | | | | fat | | | | | | | |
| 392 | 444 | IV | Ascending | 2 | T3 | G2 | Invasion | positive | 1/6 | N1 | positive | Macrovesicular | M1 | Tumor |
| | | | colon | | | | through | | | | (Liver) | and | | arising at |
| | | | | | | | muscularis | | | | | microvesicular | | prior |
| | | | | | | | propria | | | | | steatosis | | ileocolic |
| | | | | | | | into | | | | | | | surgical |
| | | | | | | | subserosal | | | | | | | anastomosis. |
| | | | | | | | adipose | | | | | | | |
| | | | | | | | tissue, | | | | | | | |
| | | | | | | | not | | | | | | | |
| | | | | | | | serosa. | | | | | | | |
| 393 | 445 | II | Cecum | 6.0 | T3 | G2 | Cecum, | negative | 0/21 | N0 | negative | | M0 | |
| | | | | | | | invades | | | | | | | |
| | | | | | | | through | | | | | | | |
| | | | | | | | muscularis | | | | | | | |
| | | | | | | | propria | | | | | | | |
| | | | | | | | to | | | | | | | |
| | | | | | | | involve | | | | | | | |
| | | | | | | | subserosal | | | | | | | |
| | | | | | | | adipose | | | | | | | |
| | | | | | | | tissue | | | | | | | |
| | | | | | | | but | | | | | | | |
| | | | | | | | not | | | | | | | |
| | | | | | | | serosa. | | | | | | | |
| 413 | 465 | IV | Ascending | 4.8 | T3 | G2 | Invasive | negative | 0/7 | N0 | positive | adenocarcinoma | M1 | rediagnosis |
| | | | colon | | | | through | | | | (Liver) | in | | of |
| | | | | | | | muscularis | | | | | multiple | | oophorectomy |
| | | | | | | | to | | | | | slides | | path |
| | | | | | | | involve | | | | | | | to |
| | | | | | | | periserosal | | | | | | | metastatic |
| | | | | | | | fat; | | | | | | | colon |
| | | | | | | | abutting | | | | | | | cancer. |
| | | | | | | | ileocecal | | | | | | | |
| | | | | | | | junction. | | | | | | | |
| 505 | 383 | IV | | 7.5 cm | T3 | G2 | Invasion | positive | 2/17 | N1 | positive | moderately | M1 | Anatomical |
| | | | | max | | | through | | | | (Liver) | differentiated | | location |
| | | | | dim | | | muscularis | | | | | adenocarcinoma, | | of primary |
| | | | | | | | propria | | | | | consistant | | not |
| | | | | | | | involving | | | | | with | | notated in |
| | | | | | | | pericolic | | | | | primary | | report. |
| | | | | | | | adipose, | | | | | | | Evidence |
| | | | | | | | serosal | | | | | | | of chronic |
| | | | | | | | surface | | | | | | | colitis. |
| | | | | | | | uninvolved | | | | | | | |
| 517 | 395 | IV | Sigmoid | 3 | T3 | G2 | penetrates | positive | 6/6 | N2 | negative | | M0 | No |
| | | | | | | | muscularis | | | | | | | mention |
| | | | | | | | propria, | | | | | | | of distant |
| | | | | | | | involves | | | | | | | met in |
| | | | | | | | pericolonic | | | | | | | report |
| | | | | | | | fat. | | | | | | | |
| 534 | 553 | II | Ascending | 12 | T3 | G3 | Invasion | negative | 0/8 | N0 | negative | | M0 | Omentum |
| | | | colon | | | | through | | | | | | | with |
| | | | | | | | the | | | | | | | fibrosis |
| | | | | | | | muscularis | | | | | | | and fat |
| | | | | | | | propria | | | | | | | necrosis. |
| | | | | | | | involving | | | | | | | Small |
| | | | | | | | pericolic | | | | | | | bowel |
| | | | | | | | fat. | | | | | | | with acute |
| | | | | | | | Serosa | | | | | | | and |
| | | | | | | | free of | | | | | | | chronic |
| | | | | | | | tumor. | | | | | | | serositis, |
| | | | | | | | | | | | | | | focal |
| | | | | | | | | | | | | | | abscess |
| | | | | | | | | | | | | | | and |
| | | | | | | | | | | | | | | adhesions. |
| 546 | 565 | IV | Ascending | 5.5 | T3 | G2 | Invasion | positive | 6/12 | N2 | positive | metastatic | M1 | |
| | | | colon | | | | through | | | | (Liver) | adenocarcinoma | | |
| | | | | | | | muscularis | | | | | | | |
| | | | | | | | propria | | | | | | | |
| | | | | | | | extensively | | | | | | | |
| | | | | | | | through | | | | | | | |
| | | | | | | | submucosal | | | | | | | |
| | | | | | | | and | | | | | | | |
| | | | | | | | extending | | | | | | | |
| | | | | | | | to | | | | | | | |
| | | | | | | | serosa. | | | | | | | |
| 577 | 596 | II | Cecum | 11.5 | T3 | G2 | Invasion | negative | 0/58 | N0 | negative | | M0 | Appendix |
| | | | | | | | through | | | | | | | dilated |
| | | | | | | | the | | | | | | | and |
| | | | | | | | bowel | | | | | | | fibrotic, |
| | | | | | | | wall, | | | | | | | but not |
| | | | | | | | into | | | | | | | involved |
| | | | | | | | suberosal | | | | | | | by tumor |
| | | | | | | | adipose. | | | | | | | |
| | | | | | | | Serosal | | | | | | | |
| | | | | | | | surface | | | | | | | |
| | | | | | | | free | | | | | | | |
| | | | | | | | of | | | | | | | |
| | | | | | | | tumor. | | | | | | | |
| 695 | 714 | II | Cecum | 14 | T3 | G2 | extending | negative | 0/22 | N0 | negative | | MX | tubular |
| | | | | | | | through | | | | | | | adenoma |
| | | | | | | | bowel | | | | | | | and |
| | | | | | | | wall | | | | | | | hyperplstic |
| | | | | | | | into | | | | | | | polyps |
| | | | | | | | serosal | | | | | | | present, |
| | | | | | | | fat | | | | | | | moderately |
| | | | | | | | | | | | | | | differentiated |
| | | | | | | | | | | | | | | adenoma |
| | | | | | | | | | | | | | | with |
| | | | | | | | | | | | | | | mucinous |
| | | | | | | | | | | | | | | diferentiation |
| | | | | | | | | | | | | | | (% not |
| | | | | | | | | | | | | | | stated) |
| 784 | 803 | IV | Ascending | 3.5 | T3 | G3 | through | positive | 5/17 | N2 | positive | | M1 | invasive |
| | | | colon | | | | muscularis | | | | (Liver) | | | poorly |
| | | | | | | | propria | | | | | | | differentiated |
| | | | | | | | into | | | | | | | adenosquamous |
| | | | | | | | pericolic | | | | | | | carcinoma |
| | | | | | | | soft | | | | | | | |
| | | | | | | | tissues | | | | | | | |
| 786 | 805 | IV | Descending | 9.5 | T3 | G2 | through | negative | 0/12 | N0 | positive | | M1 | moderately |
| | | | colon | | | | muscularis | | | | (Liver) | | | differentiated |
| | | | | | | | propria | | | | | | | invasive |
| | | | | | | | into | | | | | | | adenocarcinoma |
| | | | | | | | pericolic | | | | | | | |
| | | | | | | | fat, | | | | | | | |
| | | | | | | | but | | | | | | | |
| | | | | | | | not at | | | | | | | |
| | | | | | | | serosal | | | | | | | |
| | | | | | | | surface | | | | | | | |
| 791 | 810 | IV | Ascending | 5.8 | T3 | G3 | through | positive | 13/25 | N2 | positive | | M1 | poorly |
| | | | colon | | | | the | | | | (Liver) | | | differentiated |
| | | | | | | | muscularis | | | | | | | invasive |
| | | | | | | | propria | | | | | | | colonic |
| | | | | | | | into | | | | | | | adenocarcinoma |
| | | | | | | | pericolic | | | | | | | |
| | | | | | | | fat | | | | | | | |
| 888 | 908 | IV | Ascending | 2.0 | T2 | G1 | into | positive | 3/21 | N0 | positive | | M1 | well-to |
| | | | colon | | | | muscularis | | | | (Liver) | | | moderately- |
| | | | | | | | propria | | | | | | | differentiated |
| | | | | | | | | | | | | | | adenocarcinoma; |
| | | | | | | | | | | | | | | this |
| | | | | | | | | | | | | | | patient has |
| | | | | | | | | | | | | | | tumors of |
| | | | | | | | | | | | | | | the |
| | | | | | | | | | | | | | | ascending |
| | | | | | | | | | | | | | | colon and |
| | | | | | | | | | | | | | | the |
| | | | | | | | | | | | | | | sigmoid |
| | | | | | | | | | | | | | | colon |
| 889 | 909 | IV | Cecum | 4.8 | T3 | G2 | through | positive | 1/4 | N1 | positive | | M1 | moderately |
| | | | | | | | muscularis | | | | (Liver) | | | differentiated |
| | | | | | | | propria | | | | | | | adenocarcinoma |
| | | | | | | | int | | | | | | | |
| | | | | | | | subserosal | | | | | | | |
| | | | | | | | tissue | | | | | | | |

Identification of Differentially Expressed Genes

cDNA probes were prepared from total RNA isolated from the patient cells described above. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red), and vice versa.

Each array used had an identical spatial layout and control spot set. Each microarray was divided into two areas, each area having an array with, on each half, twelve groupings of 32×12 spots, for a total of about 9,216 spots on each array. The two areas are spotted identically which provide for at least two duplicates of each clone per array.

Polynucleotides corresponding to the differentially expressed genes described herein for use on the arrays were obtained from both publicly available sources and from cDNA libraries generated from selected cell lines and patient tissues. PCR products of from about 0.5 kb to 2.0 kb amplified from these sources were spotted onto the array using a Molecular Dynamics Gen III spotter according to the manufacturer's recommendations. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about four duplicate measurements for each clone, two of one color and two of the other, for each sample.

The differential expression assay was performed by mixing equal amounts of probes from tumor cells and normal cells of the same patient. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

The results are provided in Table 6 below. The table includes: 1) the SEQ ID NO; 2) the sample identification (Sample ID); 3) the spot identification number (“SpotID”); and 4) the percentage of patients tested in which expression levels of the gene was at least 2-fold greater in cancerous tissue than in matched normal tissue (“ColonPatients pvalcorrected 95_{—}>=2×”). The ratios of differential expression is expressed as a normalized hybridization signal associated with the tumor probe divided by the normalized hybridization signal with the normal probe. Thus, a ratio greater than 1 indicates that the gene product is increased in expression in cancerous cells relative to normal cells, while a ratio of less than 1 indicates the opposite.

TABLE 6

| | | | ColonPatients |
|---|---|---|---|
| | | Chip | pvalcorrected |
| SEQ ID NO | SampleID | Spot Id | 95_ >= 2x |
| 1 | RG:727787:Order7TM31:E07 | 29912 | 82.14 |
| 7 | M00055209C:B07 | 24297 | 30.30 |
| 9 | M00056908A:H05 | 21544 | 42.42 |
| 13 | M00057000D:E08 | 21592 | 30.30 |
| 27 | RG:1418951:Order7TM11:D12 | 33623 | 78.57 |
| 29 | RG:1418951:Order7TM11:D12 | 33623 | 78.57 |
| 22 | M00001346C:A05 | 243 | 55 |
| 22 | M00054893C:D03 | 21952 | 30 |

These data provide evidence that the genes represented by the polynucleotides having the indicated sequences are differentially expressed in colon cancer.

Those skilled in the art will recognize, or be able to ascertain, using not more than routine experimentation, many equivalents to the specific embodiments of the invention described herein. Such specific embodiments and equivalents are intended to be encompassed by the following claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Deposit Information. A deposit of biologically pure cultures of the following viruses was made with the American Type Culture Collection, 10801 University Blvd., Manassa, Va. 20110-2209, under the provisions of the Budapest Treaty, on or before the filing date of the present application. The accession number indicated was assigned after successful viability testing, and the requisite fees were paid. Access to said cultures will be available during pendency of the patent application to one determined by the Commissioner to be entitled to such under 37 C.F.R. §1.14 and 35 U.S.C. §122. All restriction on availability of said cultures to the public will be irrevocably removed upon the granting of a patent based upon the application. Moreover, the designated deposits will be maintained for a period of thirty (30) years from the date of deposit, or for five (5) years after the last request for the deposit; or for the enforceable life of the U.S. patent, whichever is longer. Should a culture become nonviable or be inadvertently destroyed, or, in the case of plasmid-containing strains, lose its plasmid, it will be replaced with a viable culture(s) of the same taxonomic description.

These deposits are provided merely as a convenience to those of skill in the art, and are not an admission that a deposit is required. The nucleic acid sequences of these plasmids, as well as the amino sequences of the polypeptides encoded thereby, are controlling in the event of any conflict with the description herein. A license may be required to make, use, or sell the deposited materials, and no such license is hereby granted.

In addition, pools of selected clones, as well as libraries containing specific clones, were assigned an “ES” number (internal reference) and deposited with the ATCC. Table 7 below provides the ATCC Accession Nos. of the deposited clones, all of which were deposited on or before the filing date of the application.

TABLE 7
Pools of Clones and Libraries Deposited with the ATCC

| Sequence Name | Clones | CMCC | ATCC |
|---|---|---|---|
| SK1 | SK-1 | 5162 | PTA-1360 |
| SK2 | SK-2 | 5163 | PTA-1361 |
| SK5 | SK-5 | 5164 | PTA-1362 |
| 1665 short | 1665 short | 5165 | PTA-1363 |
| 1665 long | 1665 long | 5166 | PTA-1363 |
| sk19 | SK-19 | 5167 | PTA-1364 |
| Junc2 | Junc2-6 | 5168 | PTA-1365 |
| XD4 | XD4b | 5169 | PTA-1366 |
| XD1 | XD1b | 5170 | PTA-1367 |
| XD7 | XD7c | 5171 | PTA-1368 |
| XD10 | XD10b | 5172 | PTA-1369 |
| XD11 | XD11b | 5173 | PTA-1370 |
| Junc4 | Junc4-2 | 5174 | PTA-1371 |

CMCC refers to applicant's internal reference number.

Retrieval of Individual Clones from Deposit of Pooled Clones. Where the ATCC deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

### Example 13

### ATCC Deposits

The following plasmids were deposited as a bacterial culture with plasmid cDNA on Sep. 25, 1998 with the American Type Culture Collection, 1301 Parklawn Drive, Rockville, Md., USA (ATCC) as ATCC accession no. 98896:

1) Clone HX2134-4 (containing an insert corresponding to SEQ ID NO:128),

2) Clone HX2144-1 (containing an insert corresponding to SEQ ID NO: 129);

3) Clone HX2145-3 (containing an insert corresponding to SEQ ID NO: 130);

4) Clone HX2162-3 (containing an insert corresponding to SEQ ID NO: 131);

5) Clone HX2166-6 (containing an insert corresponding to SEQ ID NO: 132); and

6) Clone HX2192-1 (containing an insert corresponding to SEQ ID NO:133).

The deposit was made under the conditions specified by the Budapest Treaty on the international recognition of the deposit of microorganisms (Budapest Treaty). Constructs and polynucleotides sequences equivalent to and/or substantially equivalent to the deposited material are also considered to be within the scope of this invention. Availability of the deposited material is not to be construed as a license to practice the invention in contravention of the rights granted under the authority of any government in accordance with its patent laws.

Each of the above clones was transfected into separate bacterial cells, and were deposited as a pool of equal mixtures of all six clones in this composite deposit. Each clone can be removed from the vector in which it was deposited by EcoRI to produce the appropriately sized 0.5 kb-1.0 kb fragment for the clone. Particular clones can be obtained from the composite deposit using methods well known in the art. For example, a bacterial cell containing a particular clone can be identified by isolating single colonies on an appropriate bacterial media containing ampicillin, and identifying colonies containing the specific clone through standard colony hybridization techniques, using an oligonucleotide probe or probes designed to specifically hybridize to a sequence of one of SEQ ID NOS:128-133. The probe should be designed to have a Tₘ of approximately 80 EC (assuming 2 EC for each A or T and 4 EC for each G or C). Positive colonies can then be picked, grown in culture, and the recombinant clone isolated.

### Example 14

A family was identified that had several members who had been diagnosed with pancreatic cancer. The family members also have a form of diabetes. The pathological features of disease in the family included progression from normal to metaplasia to dysplasia to cancer. Tissues were obtained from a member of the family diagnosed with pancreatic cancer and from a member of the family diagnosed with dysplasia of pancreatic cells, and primary cultures of ductal cells prepared according to methods well known in the art. Tissue was also obtained from an unrelated person who was diagnosed with pancreatitis, and from an unrelated person who had a normal pancreas, and primary cultures of ductal cells prepared according to methods well known in the art.

The Genomyx HIEROGLYPH™ mRNA profile kit for differential display analysis was used according to the manufacturer's instructions to identify genes that are differentially expressed in the various samples relative to one another. Briefly, mRNA was isolated from the primary ductal cell cultures, and subjected to reverse transcriptase polymerase chain reaction (PCR). The resulting cDNA was subjected to a differential display in which the cDNA from each of the samples were compared on a gel.

The cDNA fragment pattern in each sample was manually compared to the cDNA fragment pattern in every other sample on the gel. Those bands representing differentially expressed gene products (e.g., bands associated with relatively more or less cDNA in one sample relative to another) were cut from the gel, amplified, cloned, and sequenced. The following polynucleotide sequences (SEQ ID NOS:128-133) of cDNA fragments isolated from six such differentially displayed cDNA fragments were identified as being differentially regulated in pancreatic disease.

TABLE 8
Results of Differential Display

| | | Sequence | |
|---|---|---|---|
| SEQ ID | Clone | Length | |
| NO. | Name | (bp) | Results |
| 128 | HX2134-4 | 676 | Expression decreased in dysplasia only |
| 129 | HX2144-1 | 544 | Expression increased in cancer only |
| 130 | HX2145-3 | 432 | Expression decreased in dysplasia only |
| 131 | HX2162-3 | 493 | Expression increased in dysplasia only |
| 132 | HX2166-6 | 418 | Expression increased in dysplasia only |
| 133 | HX2192-1 | 1063 | Expression decreased in dysplasia and |
| | | | cancer |

The identification of these differentially expressed polynucleotides, as well as the correlation of the relative levels of expression of the represented differentially expressed genes with the disease states of pancreatic cancer and dysplasia, indicates that the gene products of the differentially expressed polynucleotides and genes can serve as markers of these disease states, where the markers can be used either singly or in combination with one another. Examination of expression of one or more of these differentially expressed polynucleotides can thus be used in classifying the cell from which the polynucleotides are derived as, for example, cancerous, dysplastic, or normal, and can further be used in diagnosis of the subject from whom the cell sample was derived. Use of all or a subset of the differentially expressed polynucleotides as markers will increase the sensitivity and the accuracy of the diagnosis.

### Example 15

### Sequencing and Analysis of Differentially Expressed Polynucleotides

The sequences of the differentially expressed polynucleotides identified in Example 1 (SEQ ID NOS:128-133) were used as query sequences in the GenBank and dbEST public databases to identify possible homologous sequences. The search was performed using the BLAST program, with default settings. All six sequences were novel, i.e., no sequence present in the databases searched contained a sequence having the contiguous nucleotide sequence set forth in any of SEQ ID NOS:128-133. Moreover, each of the polynucleotides contained stretches of contiguous nucleotides for which no homologous sequence was identified. A summary of these wholly unique sequences, referred to herein as identifying sequences, is provided in Table 9 below.

TABLE 9
Identifying sequences of the differentially expressed genes of
the invention.

| | Identifying Sequences |
|---|---|
| SEQ ID | (numbering refers to nucleotide |
| NO: | position in Sequence Listing) |
| 128 | 1-304; 533-571 |
| 129 | 1-62; 102-139; 183-544 |
| 130 | 1-41; 62-182; 216-281; 319-432 |
| 131 | 1-13; 32-137; 156-236; 255-429; 453-493 |
| 132 | 1-101; 408-418 |
| 133 | 327-444; 640-997; 1018-1063 |

The identifying sequences above represent exemplary minimal, contiguous nucleotides sequences of the differentially expressed polynucleotides than can be used in identification or detection of the corresponding differentially expressed genes described herein.

### Example 16

### Fabricating a DNA Array Using Polynucleotides Differentially Expressed in Pancreatic Cells

A DNA array is made by spotting DNA fragments onto glass microscope slides that are pretreated with poly-L-lysine. Spotting onto the array is accomplished by a robotic arrayer. The DNA is cross-linked to the glass by ultraviolet irradiation, and the free poly-L-lysine groups are blocked by treatment with 0.05% succinic anhydride, 50% 1-methyl-2-pyrrolidinone and 50% borate buffer.

The spots on the array are oligonucleotides synthesized on an ABI automated synthesizer. Each spot is one of the polynucleotides of SEQ ID NOS:128-133, each of which correspond to a gene that is differentially expressed in pancreatic cells according to varying disease states (e.g., overexpressed or underexpressed in cancerous, dysplastic, pancreatitis, and/or diabetic pancreatic cells). The polynucleotides may be present on the array in any of a variety of combinations or subsets. Some internal standards and negative control spots including non-differentially expressed sequences and/or bacterial controls are included.

mRNA from patient samples is isolated, the mRNA used to produce cDNA, amplified and subsequently labeled with fluorescent nucleotides as follows: isolated mRNA is added to a standard PCR reaction containing primers (100 pmoles each), 250 uM nucleotides, and 5 Units of Taq polymerase (Perkin Elmer). In addition, fluorescent nucleotides (Cy3-dUTP (green fluorescence) or Cy5-dUTP (red fluorescence), sold by Amersham) are added to a final concentration of 60 uM. The reaction is carried out in a Perkin Elmer thermocycler (PE9600) for 30 cycles using the following cycle profile: 92° C. for 30 seconds, 58° C. for 30 seconds, and 72° C. for 2 minutes. Unincorporated fluorescent nucleotides are removed by size exclusion chromatography (Microcon-30 concentration devices, sold by Amicon).

Buffer replacement, removal of small nucleotides and primers and sample concentration is accomplished by ultrafiltration over an Amicon microconcentrator-30 (mwco=30,000 Da) with three changes of 0.45 ml TE. The sample is reduced to 5 μl and supplemented with 1.4 μl 20×SSC and 5 μg yeast tRNA. Particles are removed from this mixture by filtration through a pre-wetted 0.45μ microspin filter (Ultrafree-MC, Millipore, Bedford, Ma.). SDS is added to a 0.28% final concentration. The fluorescently-labeled cDNA mixture is then heated to 98° C. for 2 min., quickly cooled and applied to the DNA array on a microscope slide. Hybridization proceeds under a coverslip, and the slide assembly is kept in a humidified chamber at 65° C. for 15 hours.

The slide is washed briefly in 1×SSC and 0.03% SDS, followed by a wash in 0.06% SSC. The slide is kept in a humidified chamber until fluorescence scanning was done. Fluorescence scanning and data acquisition are then accomplished using any of a variety of suitable methods well known in the art. For example, fluorescence scanning is set for 20 microns/pixel and two readings are taken per pixel. Data for channel 1 is set to collect fluorescence from Cy3 with excitation at 520 nm and emission at 550-600 nm. Channel 2 collects signals excited at 647 nm and emitted at 660-705 nm, appropriate for Cy5. No neutral density filters are applied to the signal from either channel, and the photomultiplier tube gain is set to 5. Fine adjustments are then made to the photomultiplier gain so that signals collected from the two spots are equivalent.

The data acquired from the scan of the array is then converted to any suitable form for analysis. For example, the data may be analyzed using a computer system, and the data may be displayed in a pictoral format on a computer screen, where the display shows the array as a collection of spots, each spot corresponding to a location of a different polynucleotide on the array. The spots vary in brightness according to the amount of fluorescent probe associated with the spot, which in turn is correlated with an amount of hybridized cDNA in the sample. The relative brightness of the spots on the array can be compared with one another to determine their relative intensities, either qualitatively or quantitatively.

The display of spots on the array, along with their relative brightness, provides a test sample pattern. The test sample pattern can be then compared with reference array patterns associated with positive and negative control samples on the same array, e.g., an array having polynucleotides in substantially the same locations as the array used with the test sample. The reference array patterns used in the comparison can be array patterns generated using samples from normal pancreas cells, cancerous pancreas cells, pancreatitis-associated pancreas cells, diabetic pancreas cells, and the like. A substantial or significant match between the test array pattern and a reference array pattern is indicative of a disease state of the patient from whom the test sample was obtained.

### Example 17

### Source of Biological Materials and Overview of Novel Polynucleotides Expressed by the Biological Materials

Candidate polynucleotides that may represent novel polynucleotides were obtained from cDNA libraries generated from selected cell lines and patient tissues. In order to obtain the candidate polynucleotides, mRNA was isolated from several selected cell lines and patient tissues, and used to construct cDNA libraries. The cells and tissues that served as sources for these cDNA libraries are summarized in Table 10 below.

Human colon cancer cell line Km12L4-A (Morikawa, et al., Cancer Research (1988) 48:6863) is derived from the KM12C cell line. The KM12C cell line (Morikawa et al. Cancer Res. (1988) 48:1943-1948), which is poorly metastatic (low metastatic) was established in culture from a Dukes' stage B2 surgical specimen (Morikawa et al. Cancer Res. (1988) 48:6863). The KM12L4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246).

The MDA-MB-231 cell line (Brinkley et al. Cancer Res. (1980) 40:3118-3129) was originally isolated from pleural effusions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. The MV-522 cell line is derived from a human lung carcinoma and is of high metastatic potential. The UCP-3 cell line is a low metastatic human lung carcinoma cell line; the MV-522 is a high metastatic variant of UCP-3. These cell lines are well-recognized in the art as models for the study of human breast and lung cancer (see, e.g., Chandrasekaran et al., Cancer Res. (1979) 39:870 (MDA-MB-231 and MCF-7); Gastpar et al., J Med Chem (1998) 41:4965 (MDA-MB-231 and MCF-7); Ranson et al., Br J Cancer (1998) 77:1586 (MDA-MB-231 and MCF-7); Kuang et al., Nucleic Acids Res (1998) 26:1116 (MDA-MB-231 and MCF-7); Varki et al., Int J Cancer (1987) 40:46 (UCP-3); Varki et al., Tumour Biol. (1990) 11:327; (MV-522 and UCP-3); Varki et al., Anticancer Res. (1990) 10:637; (MV-522); Kelner et al., Anticancer Res (1995) 15:867 (MV-522); and Zhang et al., Anticancer Drugs (1997) 8:696 (MV522)).

The samples of libraries 15-20 are derived from two different patients (UC#2, and UC#3). The bFGF-treated HMVEC were prepared by incubation with bFGF at 10 ng/ml for 2 hrs; the VEGF-treated HMVEC were prepared by incubation with 20 ng/ml VEGF for 2 hrs. Following incubation with the respective growth factor, the cells were washed and lysis buffer added for RNA preparation.

GRRpz was derived from normal prostate epithelium. The WOca cell line is a Gleason Grade 4 cell line.

The source materials for generating the normalized prostate libraries of libraries 25 and 26 were cryopreserved prostate tumor tissue from a patient with Gleason grade 3+3 adenocarcinoma and matched normal prostate biopsies from a pool of at-risk subjects under medical surveillance. The source materials for generating the normalized prostate libraries of libraries 30 and 31 were cryopreserved prostate tumor tissue from a patient with Gleason grade 4+4 adenocarcinoma and matched normal prostate biopsies from a pool of at-risk subjects under medical surveillance.

The source materials for generating the normalized breast libraries of libraries 27, 28 and 29 were cryopreserved breast tissue from a primary breast tumor (infiltrating ductal carcinoma)(library 28), from a lymph node metastasis (library 29), or matched normal breast biopsies from a pool of at-risk subjects under medical surveillance. In each case, prostate or breast epithelia were harvested directly from frozen sections of tissue by laser capture microdissection (LCM, Arcturus Enginering Inc., Mountain View, Calif.), carried out according to methods well known in the art (see, Simone et al. Am J Pathol. 156(2):445-52 (2000)), to provide substantially homogenous cell samples.

TABLE 10
Description of cDNA Libraries

| | | Number |
|---|---|---|
| Library | | of Clones in |
| (lib#) | Description | Library |
| 0 | Artificial library composed of deselected clones (clones with | 673 |
| | no associated variant or cluster) | |
| 1 | Human Colon Cell Line Km12 L4: High Metastatic Potential | 308731 |
| | (derived from Km12C) | |
| 2 | Human Colon Cell Line Km12C: Low Metastatic Potential | 284771 |
| 3 | Human Breast Cancer Cell Line MDA-MB-231: High | 326937 |
| | Metastatic Potential; micro-mets in lung | |
| 4 | Human Breast Cancer Cell Line MCF7: Non Metastatic | 318979 |
| 8 | Human Lung Cancer Cell Line MV-522: High Metastatic | 223620 |
| | Potential | |
| 9 | Human Lung Cancer Cell Line UCP-3: Low Metastatic | 312503 |
| | Potential | |
| 12 | Human microvascular endothelial cells (HMEC) - | 41938 |
| | UNTREATED (PCR (OligodT) cDNA library) | |
| 13 | Human microvascular endothelial cells (HMEC) - bFGF | 42100 |
| | TREATED (PCR (OligodT) cDNA library) | |
| 14 | Human microvascular endothelial cells (HMEC) - VEGF | 42825 |
| | TREATED (PCR (OligodT) cDNA library) | |
| 15 | Normal Colon - UC#2 Patient (MICRODISSECTED PCR | 282722 |
| | (OligodT) cDNA library) | |
| 16 | Colon Tumor - UC#2 Patient (MICRODISSECTED PCR | 298831 |
| | (OligodT) cDNA library) | |
| 17 | Liver Metastasis from Colon Tumor of UC#2 Patient | 303467 |
| | (MICRODISSECTED PCR (OligodT) cDNA library) | |
| 18 | Normal Colon - UC#3 Patient (MICRODISSECTED PCR | 36216 |
| | (OligodT) cDNA library) | |
| 19 | Colon Tumor - UC#3 Patient (MICRODISSECTED PCR | 41388 |
| | (OligodT) cDNA library) | |
| 20 | Liver Metastasis from Colon Tumor of UC#3 Patient | 30956 |
| | (MICRODISSECTED PCR (OligodT) cDNA library) | |
| 21 | GRRpz Cells derived from normal prostate epithelium | 164801 |
| 22 | WOca Cells derived from Gleason Grade 4 prostate cancer | 162088 |
| | epithelium | |
| 23 | Normal Lung Epithelium of Patient #1006 | 306198 |
| | (MICRODISSECTED PCR (OligodT) cDNA library) | |
| 24 | Primary tumor, Large Cell Carcinoma of Patient #1006 | 309349 |
| | (MICRODISSECTED PCR (OligodT) cDNA library) | |
| 25 | Normal Prostate Epithelium from Patient IF97-26811 | 279444 |
| 26 | Prostate Cancer Epithelium Gleason 3 + 3 Patient IF97-26811 | 269406 |
| 27 | Normal Breast Epithelium from Patient 515 | 239494 |
| 28 | Primary Breast tumor from Patient 515 | 259960 |
| 29 | Lymph node metastasis from Patient 515 | 326786 |
| 30 | Normal Prostate Epithelium from Chiron Patient ID 884 | 298431 |
| 31 | Prostate Cancer Epithelium (Gleason 4 + 4) from Chiron Patient | 331941 |
| | ID 884 | |

Characterization of Sequences in the Libraries

After using the software program Phred (ver 0.000925.c, Green and Weing, ©11993-2000) to select those polynucleotides having the best quality sequence, the polynucleotides were compared against the public databases to identify any homologous sequences. The sequences of the isolated polynucleotides were first masked to eliminate low complexity sequences using the RepeatMasker masking program, publicly available through a web site supported by the University of Washington (See also Smit, A. F. A. and Green, P., unpublished results). Generally, masking does not influence the final search results, except to eliminate sequences of relatively little interest due to their low complexity, and to eliminate multiple “hits” based on similarity to repetitive regions common to multiple sequences, e.g., Alu repeats.

The remaining sequences were then used in a homology search of the GenBank database using the TeraBLAST program (TimeLogic, Crystal Bay, Nev.). TeraBLAST is a version of the publicly available BLAST search algorithm developed by the National Center for Biotechnology, modified to operate at an accelerated speed with increased sensitivity on a specialized computer hardware platform. The program was run with the default parameters recommended by TimeLogic to provide the best sensitivity and speed for searching DNA and protein sequences. Sequences that exhibited greater than 70% overlap, 99% identity, and a p value of less than 1×10e-40 were discarded. Sequences from this search also were discarded if the inclusive parameters were met, but the sequence was ribosomal or vector-derived.

The resulting sequences from the previous search were classified into three groups (1, 2 and 3 below) and searched in a TeraBLASTX vs. NRP (non-redundant proteins) database search: (1) unknown (no hits in the GenBank search), (2) weak similarity (greater than 45% identity and p value of less than 1×10e-5), and (3) high similarity (greater than 60% overlap, greater than 80% identity, and p value less than 1×10e-5). Sequences having greater than 70% overlap, greater than 99% identity, and p value of less than 1×10e-40 were discarded.

The remaining sequences were classified as unknown (no hits), weak similarity, and high similarity (parameters as above). Two searches were performed on these sequences. First, a TeraBLAST vs. EST database search was performed and sequences with greater than 99% overlap, greater than 99% similarity and a p value of less than 1×10e-40 were discarded. Sequences with a p value of less than 1×10e-65 when compared to a database sequence of human origin were also excluded. Second, a TeraBLASTN vs. Patent GeneSeq database was performed and sequences having greater than 99% identity, p value less than 1×10e-40, and greater than 99% overlap were discarded.

The remaining sequences were subjected to screening using other rules and redundancies in the dataset. Sequences with a p value of less than 1×10e-111 in relation to a database sequence of human origin were specifically excluded. The final result provided the sequences listed as SEQ ID NOS:134-1352 in the accompanying Sequence Listing and summarized in Table 11. Each identified polynucleotide represents sequence from at least a partial mRNA transcript.

TABLE 11

| SEQ | | | | |
|---|---|---|---|---|
| ID | CLUSTER | SEQ NAME | CLONE ID | LIBRARY |
| 134 | 357367 | 3538.O24.GZ43_504925 | M00084399B:E05 | chiron(cc187-NormBPHProstate) |
| 135 | 725997 | 3538.P11.GZ43_504718 | M00084400A:B09 | chiron(cc187-NormBPHProstate) |
| 136 | 645986 | 3541.A04.GZ43_504975 | M00084406A:B03 | chiron(cc187-NormBPHProstate) |
| 137 | 407828 | 3541.A05.GZ43_504991 | M00084407A:H09 | chiron(cc187-NormBPHProstate) |
| 138 | 649117 | 3541.A16.GZ43_505167 | M00084421C:B11 | chiron(cc187-NormBPHProstate) |
| 139 | 424678 | 3541.A23.GZ43_505279 | M00084431C:G08 | chiron(cc187-NormBPHProstate) |
| 140 | 854288 | 3541.B04.GZ43_504976 | M00084406C:A01 | chiron(cc187-NormBPHProstate) |
| 141 | 639901 | 3541.B17.GZ43_505184 | M00084424A:G07 | chiron(cc187-NormBPHProstate) |
| 142 | 842265 | 3538.G08.GZ43_504661 | M00084379D:A05 | chiron(cc187-NormBPHProstate) |
| 143 | 557717 | 3538.G17.GZ43_504805 | M00084380C:C09 | chiron(cc187-NormBPHProstate) |
| 144 | 459967 | 3538.G19.GZ43_504837 | M00084380D:B07 | chiron(cc187-NormBPHProstate) |
| 145 | 505750 | 3538.G22.GZ43_504885 | M00084381C:A05 | chiron(cc187-NormBPHProstate) |
| 146 | 1053564 | 3538.H05.GZ43_504614 | M00084382A:D06 | chiron(cc187-NormBPHProstate) |
| 147 | 542301 | 3538.H21.GZ43_504870 | M00084383B:A11 | chiron(cc187-NormBPHProstate) |
| 148 | 21446 | 3538.I08.GZ43_504663 | M00084385A:D02 | chiron(cc187-NormBPHProstate) |
| 149 | 1140418 | 3538.I13.GZ43_504743 | M00084385B:D03 | chiron(cc187-NormBPHProstate) |
| 150 | 530453 | 3538.J22.GZ43_504888 | M00084388A:G03 | chiron(cc187-NormBPHProstate) |
| 151 | 1204782 | 3538.K12.GZ43_504729 | M00084389A:F12 | chiron(cc187-NormBPHProstate) |
| 152 | 863475 | 3538.K23.GZ43_504905 | M00084390B:H04 | chiron(cc187-NormBPHProstate) |
| 153 | 452124 | 3538.L16.GZ43_504794 | M00084391B:D06 | chiron(cc187-NormBPHProstate) |
| 154 | 650520 | 3538.M02.GZ43_504571 | M00084392C:D03 | chiron(cc187-NormBPHProstate) |
| 155 | 1117771 | 3538.M05.GZ43_504619 | M00084392C:G06 | chiron(cc187-NormBPHProstate) |
| 156 | 434074 | 3538.M08.GZ43_504667 | M00084393A:G07 | chiron(cc187-NormBPHProstate) |
| 157 | 866609 | 3538.N20.GZ43_504860 | M00084396B:B03 | chiron(cc187-NormBPHProstate) |
| 158 | 945247 | 3538.O07.GZ43_504653 | M00084397D:A09 | chiron(cc187-NormBPHProstate) |
| 159 | 1053623 | 3541.E11.GZ43_505091 | M00084415C:C05 | chiron(cc187-NormBPHProstate) |
| 160 | 722878 | 3541.E14.GZ43_505139 | M00084419C:A09 | chiron(cc187-NormBPHProstate) |
| 161 | 1226493 | 3541.E15.GZ43_505155 | M00084420C:D03 | chiron(cc187-NormBPHProstate) |
| 162 | 812031 | 3541.G17.GZ43_505189 | M00084423C:G11 | chiron(cc187-NormBPHProstate) |
| 163 | 725997 | 3541.H14.GZ43_505142 | M00084420A:G02 | chiron(cc187-NormBPHProstate) |
| 164 | 1191547 | 3541.I15.GZ43_505159 | M00084420D:C07 | chiron(cc187-NormBPHProstate) |
| 165 | 21370 | 3541.I17.GZ43_505191 | M00084423D:B05 | chiron(cc187-NormBPHProstate) |
| 166 | 418320 | 3541.I18.GZ43_505207 | M00084424D:G07 | chiron(cc187-NormBPHProstate) |
| 167 | 24580 | 3541.J19.GZ43_505224 | M00084427B:D01 | chiron(cc187-NormBPHProstate) |
| 168 | 647587 | 3541.K09.GZ43_505065 | M00084413C:A11 | chiron(cc187-NormBPHProstate) |
| 169 | 1225989 | 3541.L19.GZ43_505226 | M00084427C:D04 | chiron(cc187-NormBPHProstate) |
| 170 | 1079863 | 3541.M02.GZ43_504955 | M00084403D:D04 | chiron(cc187-NormBPHProstate) |
| 171 | 1136803 | 3541.M07.GZ43_505035 | M00084410C:F10 | chiron(cc187-NormBPHProstate) |
| 172 | 528281 | 3541.M18.GZ43_505211 | M00084425A:A01 | chiron(cc187-NormBPHProstate) |
| 173 | 660907 | 3541.O04.GZ43_504989 | M00084406B:C03 | chiron(cc187-NormBPHProstate) |
| 174 | 402588 | 3541.O13.GZ43_505133 | M00084418D:A04 | chiron(cc187-NormBPHProstate) |
| 175 | 947168 | 3541.O23.GZ43_505293 | M00084432B:C05 | chiron(cc187-NormBPHProstate) |
| 176 | 1223948 | 3541.P05.GZ43_505006 | M00084408D:E06 | chiron(cc187-NormBPHProstate) |
| 177 | 426138 | 3541.P22.GZ43_505278 | M00084431C:B02 | chiron(cc187-NormBPHProstate) |
| 178 | 1037887 | 3544.A09.GZ43_505439 | M00084447D:F03 | chiron(cc187-NormBPHProstate) |
| 179 | 468334 | 3544.A13.GZ43_505503 | M00084454A:G08 | cbiron(cc187-NormBPHProstate) |
| 180 | 1140409 | 3544.A14.GZ43_505519 | M00084456A:H04 | chiron(cc187-NormBPHProstate) |
| 181 | 555726 | 3544.A17.GZ43_505567 | M00084463A:B07 | chiron(cc187-NormBPHProstate) |
| 182 | 726922 | 3544.B02.GZ43_505328 | M00084437C:G05 | chiron(cc187-NormBPHProstate) |
| 183 | 402516 | 3544.B09.GZ43_505440 | M00084448B:D11 | chiron(cc187-NormBPHProstate) |
| 184 | 812031 | 3544.B18.GZ43_505584 | M00084467A:D06 | chiron(cc187-NormBPHProstate) |
| 185 | 448177 | 3544.E05.GZ43_505379 | M00084441D:E09 | chiron(cc187-NormBPHProstate) |
| 186 | 505750 | 3544.E18.GZ43_505587 | M00084466B:E01 | chiron(cc187-NormBPHProstate) |
| 187 | 508322 | 3544.F06.GZ43_505396 | M00084443C:H06 | chiron(cc187-NormBPHProstate) |
| 188 | 1224072 | 3544.F16.GZ43_505556 | M00084461C:D06 | chiron(cc187-NormBPHProstate) |
| 189 | 801 | 3544.G06.GZ43_505397 | M00084443A:E10 | chiron(cc187-NormBPHProstate) |
| 190 | 748101 | 3544.G10.GZ43_505461 | M00084449A:D09 | chiron(cc187-NormBPHProstate) |
| 191 | 1224107 | 3544.G11.GZ43_505477 | M00084450C:A09 | chiron(cc187-NormBPHProstate) |
| 192 | 1226845 | 3544.G12.GZ43_505493 | M00084452B:F07 | chiron(cc187-NormBPHProstate) |
| 193 | 1073767 | 3544.H03.GZ43_505350 | M00084439B:A08 | chiron(cc187-NormBPHProstate) |
| 194 | 1224752 | 3544.H15.GZ43_505542 | M00084459A:F10 | chiron(cc187-NormBPHProstate) |
| 195 | 1052480 | 3544.H24.GZ43_505686 | M00084434B:E06 | chiron(cc187-NormBPHProstate) |
| 196 | 245031 | 3544.I07.GZ43_505415 | M00084444D:F09 | chiron(cc187-NormBPHProstate) |
| 197 | 494499 | 3544.I15.GZ43_505543 | M00084458A:G06 | chiron(cc187-NormBPHProstate) |
| 198 | 1138593 | 3544.I20.GZ43_505623 | M00084469A:C09 | chiron(cc187-NormBPHProstate) |
| 199 | 1139691 | 3544.J04.GZ43_505368 | M00084441B:E05 | chiron(cc187-NormBPHProstate) |
| 200 | 790693 | 3544.J11.GZ43_505480 | M00084451D:A03 | chiron(cc187-NormBPHProstate) |
| 201 | 1117003 | 3544.J13.GZ43_505512 | M00084455D:B03 | chiron(cc187-NormBPHProstate) |
| 202 | 844740 | 3544.J23.GZ43_505672 | M00084475B:D03 | chiron(cc187-NormBPHProstate) |
| 203 | 452564 | 3544.K16.GZ43_505561 | M00084460D:B04 | chiron(cc187-NormBPHProstate) |
| 204 | 862823 | 3544.L11.GZ43_505482 | M00084451D:F06 | chiron(cc187-NormBPHProstate) |
| 205 | 19367 | 3544.L13.GZ43_505514 | M00084455D:G03 | chiron(cc187-NormBPHProstate) |
| 206 | 1194656 | 3544.M06.GZ43_505403 | M00084443B:C02 | chiron(cc187-NormBPHProstate) |
| 207 | 1224879 | 3544.M10.GZ43_505467 | M00084449B:C09 | chiron(cc187-NormBPHProstate) |
| 208 | 454904 | 3544.N07.GZ43_505420 | M00084446A:A05 | chiron(cc187-NormBPHProstate) |
| 209 | 676665 | 3544.N12.GZ43_505500 | M00084453D:B12 | chiron(cc187-NormBPHProstate) |
| 210 | 542825 | 3544.N19.GZ43_505612 | M00084468C:E07 | chiron(cc187-NormBPHProstate) |
| 211 | 411960 | 3544.O03.GZ43_505357 | M00084438D:H04 | chiron(cc187-NormBPHProstate) |
| 212 | 936795 | 3544.O10.GZ43_505469 | M00084449C:C01 | chiron(cc187-NormBPHProstate) |
| 213 | 1283437 | 3544.O15.GZ43_505549 | M00084458B:G05 | chiron(cc187-NormBPHProstate) |
| 214 | 402150 | 3544.O20.GZ43_505629 | M00084469B:F08 | chiron(cc187-NormBPHProstate) |
| 215 | 454733 | 3544.P18.GZ43_505598 | M00084468A:A09 | chiron(cc187-NormBPHProstate) |
| 216 | 1211032 | 3547.A04.GZ43_505743 | M00084483A:C06 | chiron(cc187-NormBPHProstate) |
| 217 | 452763 | 3547.A11.GZ43_505855 | M00084493A:E03 | chiron(cc187-NormBPHProstate) |
| 218 | 528281 | 3547.A24.GZ43_506063 | M00084475C:G11 | chiron(cc187-NormBPHProstate) |
| 219 | 1136803 | 3547.C05.GZ43_505761 | M00084484C:B11 | chiron(cc187-NormBPHProstate) |
| 220 | 454826 | 3547.C17.GZ43_505953 | M00084500D:B11 | chiron(cc187-NormBPHProstate) |
| 221 | 1054807 | 3547.C23.GZ43_506049 | M00084510D:D05 | chiron(cc187-NormBPHProstate) |
| 222 | 726386 | 3547.D19.GZ43_505986 | M00084504C:F05 | chiron(cc187-NormBPHProstate) |
| 223 | 1223705 | 3547.D23.GZ43_506050 | M00084511D:A02 | chiron(cc187-NormBPHProstate) |
| 224 | 398439 | 3547.E04.GZ43_505747 | M00084483A:E05 | chiron(cc187-NormBPHProstate) |
| 225 | 833174 | 3547.F02.GZ43_505716 | M00084480B:A05 | chiron(cc187-NormBPHProstate) |
| 226 | 500919 | 3547.F10.GZ43_505844 | M00084492B:F03 | chiron(cc187-NormBPHProstate) |
| 227 | 1226064 | 3547.F20.GZ43_506004 | M00084506A:E08 | chiron(cc187-NormBPHProstate) |
| 228 | 555509 | 3547.G02.GZ43_505717 | M00084479B:E04 | chiron(cc187-NormBPHProstate) |
| 229 | 653817 | 3547.G09.GZ43_505829 | M00084490A:C12 | chiron(cc187-NormBPHProstate) |
| 230 | 478212 | 3547.G22.GZ43_506037 | M00084509D:C02 | chiron(cc187-NormBPHProstate) |
| 231 | 1054074 | 3547.H12.GZ43_505878 | M00084495B:C11 | chiron(cc187-NormBPHProstate) |
| 232 | 1060021 | 3547.H14.GZ43_505910 | M00084497D:D03 | chiron(cc187-NormBPHProstate) |
| 233 | 1227352 | 3547.I07.GZ43_505799 | M00084487C:H06 | chiron(cc187-NormBPHProstate) |
| 234 | 8293 | 3547.I16.GZ43_505943 | M00084499C:C11 | chiron(cc187-NormBPHProstate) |
| 235 | 477110 | 3547.I17.GZ43_505959 | M00084501A:D06 | chiron(cc187-NormBPHProstate) |
| 236 | 1224039 | 3547.I20.GZ43_506007 | M00084505C:H08 | chiron(cc187-NormBPHProstate) |
| 237 | 542301 | 3547.J05.GZ43_505768 | M00084485C:B04 | chiron(cc187-NormBPHProstate) |
| 238 | 455211 | 3547.J10.GZ43_505848 | M00084492C:B05 | chiron(cc187-NormBPHProstate) |
| 239 | 1056369 | 3547.J20.GZ43_506008 | M00084506C:A05 | chiron(cc187-NormBPHProstate) |
| 240 | 549814 | 3547.J22.GZ43_506040 | M00084510C:F02 | chiron(cc187-NormBPHProstate) |
| 241 | 509673 | 3547.K01.GZ43_505705 | M00084477C:C07 | chiron(cc187-NormBPHProstate) |
| 242 | 736256 | 3547.L09.GZ43_505834 | M00084491A:E08 | chiron(cc187-NormBPHProstate) |
| 243 | 1139849 | 3547.L11.GZ43_505866 | M00084494C:C01 | chiron(cc187-NormBPHProstate) |
| 244 | 1223938 | 3547.L16.GZ43_505946 | M00084500C:D01 | chiron(cc187-NormBPHProstate) |
| 245 | 1059445 | 3547.L22.GZ43_506042 | M00084510C:F05 | chiron(cc187-NormBPHProstate) |
| 246 | 478212 | 3547.M02.GZ43_505723 | M00084479D:E10 | chiron(cc187-NormBPHProstate) |
| 247 | 1140418 | 3547.M07.GZ43_505803 | M00084487D:F04 | chiron(cc187-NormBPHProstate) |
| 248 | 534943 | 3547.M08.GZ43_505819 | M00084489A:D12 | chiron(cc187-NormBPHProstate) |
| 249 | 708025 | 3547.M16.GZ43_505947 | M00084499D:A10 | chiron(cc187-NormBPHProstate) |
| 250 | 1138419 | 3547.N06.GZ43_505788 | M00084487B:A06 | chiron(cc187-NormBPHProstate) |
| 251 | 1226588 | 3547.O03.GZ43_505741 | M00084481D:C06 | chiron(cc187-NormBPHProstate) |
| 252 | 461669 | 3547.O07.GZ43_505805 | M00084487D:F07 | chiron(cc187-NormBPHProstate) |
| 253 | 1060021 | 3547.O14.GZ43_505917 | M00084497B:C12 | chiron(cc187-NormBPHProstate) |
| 254 | 307985 | 3547.P18.GZ43_505982 | M00084503D:G10 | chiron(cc187-NormBPHProstate) |
| 255 | 840852 | 3547.P21.GZ43_506030 | M00084509A:E10 | chiron(cc187-NormBPHProstate) |
| 256 | 402286 | 3547.P22.GZ43_506046 | M00084510C:H01 | chiron(cc187-NormBPHProstate) |
| 257 | 451994 | 3550.A12.GZ43_506255 | M00084513C:C10 | chiron(cc187-NormBPHProstate) |
| 258 | 451679 | 3550.A16.GZ43_506319 | M00084514A:A03 | chiron(cc187-NormBPHProstate) |
| 259 | 450607 | 3550.B06.GZ43_506160 | M00084515D:G03 | chiron(cc187-NormBPHProstate) |
| 260 | 887560 | 3550.C01.GZ43_506081 | M00084517C:D06 | chiron(cc187-NormBPHProstate) |
| 261 | 727396 | 3550.C22.GZ43_506417 | M00084519B:D01 | chiron(cc187-NormBPHProstate) |
| 262 | 1224379 | 3550.D16.GZ43_506322 | M00084520B:A12 | chiron(cc187-NormBPHProstate) |
| 263 | 1137096 | 3550.D23.GZ43_506434 | M00084521A:E11 | chiron(cc187-NormBPHProstate) |
| 264 | 1052466 | 3550.E02.GZ43_506099 | M00084521B:E11 | chiron(cc187-NormBPHProstate) |
| 265 | 1064975 | 3550.E06.GZ43_506163 | M00084521C:H11 | chiron(cc187-NormBPHProstate) |
| 266 | 180092 | 3550.F06.GZ43_506164 | M00084523C:A05 | chiron(cc187-NormBPHProstate) |
| 267 | 1224269 | 3550.F08.GZ43_506196 | M00084523C:C10 | chiron(cc187-NormBPHProstate) |
| 268 | 1053564 | 3550.F20.GZ43_506388 | M00084524D:D02 | chiron(cc187-NormBPHProstate) |
| 269 | 677858 | 3550.F22.GZ43_506420 | M00084525A:E08 | chiron(cc187-NormBPHProstate) |
| 270 | 1225500 | 3550.G02.GZ43_506101 | M00084525D:H01 | chiron(cc187-NormBPHProstate) |
| 271 | 1054038 | 3550.G08.GZ43_506197 | M00084526C:G09 | chiron(cc187-NormBPHProstate) |
| 272 | 1037887 | 3550.G10.GZ43_506229 | M00084526D:E09 | chiron(cc187-NormBPHProstate) |
| 273 | 964080 | 3550.G15.GZ43_506309 | M00084527C:H07 | chiron(cc187-NormBPHProstate) |
| 274 | 553897 | 3550.G23.GZ43_506437 | M00084528C:F06 | chiron(cc187-NormBPHProstate) |
| 275 | 755391 | 3550.H10.GZ43_506230 | M00084530D:G07 | chiron(cc187-NormBPHProstate) |
| 276 | 644174 | 3550.H21.GZ43_506406 | M00084533A:C04 | chiron(cc187-NormBPHProstate) |
| 277 | 893981 | 3550.H23.GZ43_506438 | M00084533B:B10 | chiron(cc187-NormBPHProstate) |
| 278 | 821536 | 3550.I03.GZ43_506119 | M00084534B:E12 | chiron(cc187-NormBPHProstate) |
| 279 | 1227336 | 3550.I19.GZ43_506375 | M00084535D:C12 | chiron(cc187-NormBPHProstate) |
| 280 | 991366 | 3550.I21.GZ43_506407 | M00084536B:A03 | chiron(cc187-NormBPHProstate) |
| 281 | 549814 | 3550.J05.GZ43_506152 | M00084536D:F07 | chiron(cc187-NormBPHProstate) |
| 282 | 402588 | 3550.J11.GZ43_506248 | M00084537B:C05 | chiron(cc187-NormBPHProstate) |
| 283 | 710194 | 3550.K05.GZ43_506153 | M00084539D:D11 | chiron(cc187-NormBPHProstate) |
| 284 | 1222709 | 3550.K09.GZ43_506217 | M00084540B:B08 | chiron(cc187-NormBPHProstate) |
| 285 | 1053764 | 3550.K14.GZ43_506297 | M00084540D:B12 | chiron(cc187-NormBPHProstate) |
| 286 | 1062537 | 3550.L16.GZ43_506330 | M00084545C:C05 | chiron(cc187-NormBPHProstate) |
| 287 | 964593 | 3550.L19.GZ43_506378 | M00084546C:C06 | chiron(cc187-NormBPHProstate) |
| 288 | 1054038 | 3550.L23.GZ43_506442 | M00084547B:B10 | chiron(cc187-NormBPHProstate) |
| 289 | 1223477 | 3550.M21.GZ43_506411 | M00084553B:F04 | chiron(cc187-NormBPHProstate) |
| 290 | 402516 | 3550.N01.GZ43_506092 | M00084553D:G05 | chiron(cc187-NormBPHProstate) |
| 291 | 517014 | 3550.N07.GZ43_506188 | M00084554C:D05 | chiron(cc187-NormBPHProstate) |
| 292 | 1223505 | 3550.O03.GZ43_506125 | M00084558D:A04 | chiron(cc187-NormBPHProstate) |
| 293 | 872787 | 3550.O04.GZ43_506141 | M00084558D:G08 | chiron(cc187-NormBPHProstate) |
| 294 | 405366 | 3550.O08.GZ43_506205 | M00084559B:F10 | chiron(cc187-NormBPHProstate) |
| 295 | 48343 | 3550.O15.GZ43_506317 | M00084560A:G08 | chiron(cc187-NormBPHProstate) |
| 296 | 1074160 | 3550.O17.GZ43_506349 | M00084560B:F12 | chiron(cc187-NormBPHProstate) |
| 297 | 1225719 | 3550.O18.GZ43_506365 | M00084560C:G05 | chiron(cc187-NormBPHProstate) |
| 298 | 856703 | 3550.O21.GZ43_506413 | M00084561C:D07 | chiron(cc187-NormBPHProstate) |
| 299 | 970165 | 3550.P18.GZ43_506366 | M00084565A:D10 | chiron(cc187-NormBPHProstate) |
| 300 | 494890 | 3550.P23.GZ43_506446 | M00084565D:F08 | chiron(cc187-NormBPHProstate) |
| 301 | 1285039 | 3553.A09.GZ43_506591 | M00084587C:A07 | chiron(cc187-NormBPHProstate) |
| 302 | 1200453 | 3553.B07.GZ43_506560 | M00084584B:G07 | chiron(cc187-NormBPHProstate) |
| 303 | 1219617 | 3553.B16.GZ43_506704 | M00084602D:B09 | chiron(cc187-NormBPHProstate) |
| 304 | 1042918 | 3553.B22.GZ43_506800 | M00084612C:B01 | chiron(cc187-NormBPHProstate) |
| 305 | 1226086 | 3553.D04.GZ43_506514 | M00084576A:E12 | chiron(cc187-NormBPHProstate) |
| 306 | 861025 | 3553.D07.GZ43_506562 | M00084584B:H12 | chiron(cc187-NormBPHProstate) |
| 307 | 1224505 | 3553.D14.GZ43_506674 | M00084598D:H05 | chiron(cc187-NormBPHProstate) |
| 308 | 679724 | 3553.D19.GZ43_506754 | M00084605D:G09 | chiron(cc187-NormBPHProstate) |
| 309 | 234667 | 3553.E08.GZ43_506579 | M00084585D:H12 | chiron(cc187-NormBPHProstate) |
| 310 | 448576 | 3553.E09.GZ43_506595 | M00084587C:G07 | chiron(cc187-NormBPHProstate) |
| 311 | 450607 | 3553.F12.GZ43_506644 | M00084595C:C07 | chiron(cc187-NormBPHProstate) |
| 312 | 452322 | 3553.F13.GZ43_506660 | M00084597A:F06 | chiron(cc187-NormBPHProstate) |
| 313 | 1225384 | 3553.F19.GZ43_506756 | M00084607A:B03 | chiron(cc187-NormBPHProstate) |
| 314 | 974223 | 3553.G05.GZ43_506533 | M00084578B:E12 | chiron(cc187-NormBPHProstate) |
| 315 | 845715 | 3553.G06.GZ43_506549 | M00084581B:E06 | chiron(cc187-NormBPHProstate) |
| 316 | 1138997 | 3553.G07.GZ43_506565 | M00084583D:H12 | chiron(cc187-NormBPHProstate) |
| 317 | 478212 | 3553.G21.GZ43_506789 | M00084609C:F10 | chiron(cc187-NormBPHProstate) |
| 318 | 867148 | 3553.H06.GZ43_506550 | M00084582C:H03 | chiron(cc187-NormBPHProstate) |
| 319 | 1214202 | 3553.H09.GZ43_506598 | M00084588B:D02 | chiron(cc187-NormBPHProstate) |
| 320 | 585899 | 3553.H21.GZ43_506790 | M00084610D:H04 | chiron(cc187-NormBPHProstate) |
| 321 | 863475 | 3553.I13.GZ43_506663 | M00084596D:E10 | chiron(cc187-NormBPHProstate) |
| 322 | 725982 | 3553.I16.GZ43_506711 | M00084602C:E04 | chiron(cc187-NormBPHProstate) |
| 323 | 17075 | 3553.J12.GZ43_506648 | M00084595D:D08 | chiron(cc187-NormBPHProstate) |
| 324 | 1054813 | 3553.J14.GZ43_506680 | M00084599D:C02 | chiron(cc187-NormBPHProstate) |
| 325 | 1064975 | 3553.J16.GZ43_506712 | M00084603A:B07 | chiron(cc187-NormBPHProstate) |
| 326 | 1055089 | 3553.J17.GZ43_506728 | M00084604A:D02 | chiron(cc187-NormBPHProstate) |
| 327 | 551848 | 3553.J22.GZ43_506808 | M00084613A:A01 | chiron(cc187-NormBPHProstate) |
| 328 | 585899 | 3553.J24.GZ43_506840 | M00084568D:A02 | chiron(cc187-NormBPHProstate) |
| 329 | 1211899 | 3553.K01.GZ43_506473 | M00084569D:B04 | chiron(cc187-NormBPHProstate) |
| 330 | 460499 | 3553.K02.GZ43_506489 | M00084571C:D05 | chiron(cc187-NormBPHProstate) |
| 331 | 39115 | 3553.K03.GZ43_506505 | M00084573D:G11 | chiron(cc187-NormBPHProstate) |
| 332 | 242901 | 3553.K05.GZ43_506537 | M00084578C:G09 | chiron(cc187-NormBPHProstate) |
| 333 | 719892 | 3553.K07.GZ43_506569 | M00084584B:A02 | chiron(cc187-NormBPHProstate) |
| 334 | 1085638 | 3553.K15.GZ43_506697 | M00084600D:B10 | chiron(cc187-NormBPHProstate) |
| 335 | 449465 | 3538.A11.GZ43_504703 | M00084363A:C02 | chiron(cc187-NormBPHProstate) |
| 336 | 542825 | 3538.A24.GZ43_504911 | M00084364C:B06 | chiron(cc187-NormBPHProstate) |
| 337 | 1065531 | 3538.B01.GZ43_504544 | M00084364D:F08 | chiron(cc187-NormBPHProstate) |
| 338 | 985859 | 3538.B20.GZ43_504848 | M00084367D:E06 | chiron(cc187-NormBPHProstate) |
| 339 | 409182 | 3538.C01.GZ43_504545 | M00084368D:C02 | chiron(cc187-NormBPHProstate) |
| 340 | 1223271 | 3538.C02.GZ43_504561 | M00084368D:D03 | chiron(cc187-NormBPHProstate) |
| 341 | 445742 | 3538.D06.GZ43_504626 | M00084372D:H11 | chiron(cc187-NormBPHProstate) |
| 342 | 451679 | 3538.D09.GZ43_504674 | M00084373A:F08 | chiron(cc187-NormBPHProstate) |
| 343 | 1141371 | 3538.D21.GZ43_504866 | M00084374A:A10 | chiron(cc187-NormBPHProstate) |
| 344 | 1014734 | 3538.E15.GZ43_504771 | M00084376A:E06 | chiron(cc187-NormBPHProstate) |
| 345 | 1226932 | 3538.F02.GZ43_504564 | M00084377B:E11 | chiron(cc187-NormBPHProstate) |
| 346 | 1227303 | 3538.F08.GZ43_504660 | M00084377D:E08 | chiron(cc187-NormBPHProstate) |
| 347 | 561390 | 3553.K23.GZ43_506825 | M00084614C:G05 | chiron(cc187-NormBPHProstate) |
| 348 | 529709 | 3553.K24.GZ43_506841 | M00084567B:F03 | chiron(cc187-NormBPHProstate) |
| 349 | 1227781 | 3553.L02.GZ43_506490 | M00084572D:F07 | chiron(cc187-NormBPHProstate) |
| 350 | 1138572 | 3553.L04.GZ43_506522 | M00084577B:C08 | chiron(cc187-NormBPHProstate) |
| 351 | 1117003 | 3553.L21.GZ43_506794 | M00084611A:A06 | chiron(cc187-NormBPHProstate) |
| 352 | 1228033 | 3553.M12.GZ43_506651 | M00084595B:C08 | chiron(cc187-NormBPHProstate) |
| 353 | 961119 | 3553.M23.GZ43_506827 | M00084614D:A08 | chiron(cc187-NormBPHProstate) |
| 354 | 611592 | 3553.N01.GZ43_506476 | M00084571A:C02 | chiron(cc187-NormBPHProstate) |
| 355 | 555115 | 3553.N02.GZ43_506492 | M00084573A:A10 | chiron(cc187-NormBPHProstate) |
| 356 | 856703 | 3553.N04.GZ43_506524 | M00084577B:D04 | chiron(cc187-NormBPHProstate) |
| 357 | 846056 | 3553.N07.GZ43_506572 | M00084585B:D06 | chiron(cc187-NormBPHProstate) |
| 358 | 640277 | 3553.N08.GZ43_506588 | M00084587B:H07 | chiron(cc187-NormBPHProstate) |
| 359 | 214192 | 3553.O07.GZ43_506573 | M00084584B:F09 | chiron(cc187-NormBPHProstate) |
| 360 | 1226160 | 3553.O18.GZ43_506749 | M00084604D:D08 | chiron(cc187-NormBPHProstate) |
| 361 | 1227968 | 3553.O23.GZ43_506829 | M00084614D:B07 | chiron(cc187-NormBPHProstate) |
| 362 | 1224269 | 3553.P03.GZ43_506510 | M00084575A:A11 | chiron(cc187-NormBPHProstate) |
| 363 | 774520 | 3553.P05.GZ43_506542 | M00084580B:B05 | chiron(cc187-NormBPHProstate) |
| 364 | 1227457 | 3553.P12.GZ43_506654 | M00084596A:G03 | chiron(cc187-NormBPHProstate) |
| 365 | 1110143 | 3553.P18.GZ43_506750 | M00084605B:H04 | chiron(cc187-NormBPHProstate) |
| 366 | 554189 | 3553.P21.GZ43_506798 | M00084611B:A11 | chiron(cc187-NormBPHProstate) |
| 367 | 4745 | 3556.A03.GZ43_506879 | M00084620D:E05 | chiron(cc187-NormBPHProstate) |
| 368 | 1194731 | 3556.A06.GZ43_506927 | M00084633B:A06 | chiron(cc187-NormBPHProstate) |
| 369 | 970165 | 3556.B06.GZ43_506928 | M00084634A:D01 | chiron(cc187-NormBPHProstate) |
| 370 | 1224422 | 3556.B09.GZ43_506976 | M00084640D:A08 | chiron(cc187-NormBPHProstate) |
| 371 | 613411 | 3556.B10.GZ43_506992 | M00084642C:F10 | chiron(cc187-NormBPHProstate) |
| 372 | 1056369 | 3556.B14.GZ43_507056 | M00084647C:E12 | chiron(cc187-NormBPHProstate) |
| 373 | 84347 | 3556.C13.GZ43_507041 | M00084645D:G02 | chiron(cc187-NormBPHProstate) |
| 374 | 1138593 | 3556.C15.GZ43_507073 | M00084648A:F08 | chiron(cc187-NormBPHProstate) |
| 375 | 845715 | 3556.C18.GZ43_507121 | M00084654A:E04 | chiron(cc187-NormBPHProstate) |
| 376 | 971226 | 3556.C24.GZ43_507217 | M00084615D:H12 | chiron(cc187-NormBPHProstate) |
| 377 | 1138593 | 3556.D15.GZ43_507074 | M00084648D:F05 | chiron(cc187-NormBPHProstate) |
| 378 | 413505 | 3556.D20.GZ43_507154 | M00084657C:E01 | chiron(cc187-NormBPHProstate) |
| 379 | 1224107 | 3556.D23.GZ43_507202 | M00084666A:C04 | chiron(cc187-NormBPHProstate) |
| 380 | 774520 | 3556.E13.GZ43_507043 | M00084646A:D02 | chiron(cc187-NormBPHProstate) |
| 381 | 573169 | 3556.E24.GZ43_507219 | M00084616A:G03 | chiron(cc187-NormBPHProstate) |
| 382 | 1053417 | 3556.F10.GZ43_506996 | M00084642D:E08 | chiron(cc187-NormBPHProstate) |
| 383 | 710194 | 3556.G15.GZ43_507077 | M00084648B:F06 | chiron(cc187-NormBPHProstate) |
| 384 | 558484 | 3556.H01.GZ43_506854 | M00084618C:A03 | chiron(cc187-NormBPHProstate) |
| 385 | 1211899 | 3556.H02.GZ43_506870 | M00084620A:E08 | chiron(cc187-NormBPHProstate) |
| 386 | 823271 | 3556.H12.GZ43_507030 | M00084645C:F07 | chiron(cc187-NormBPHProstate) |
| 387 | 376900 | 3556.H20.GZ43_507158 | M00084657D:B10 | chiron(cc187-NormBPHProstate) |
| 388 | 726584 | 3556.I02.GZ43_506871 | M00084619A:E04 | chiron(cc187-NormBPHProstate) |
| 389 | 725982 | 3556.I14.GZ43_507063 | M00084647C:A05 | chiron(cc187-NormBPHProstate) |
| 390 | 1211899 | 3556.J05.GZ43_506920 | M00084633A:B12 | chiron(cc187-NormBPHProstate) |
| 391 | 857031 | 3556.J07.GZ43_506952 | M00084636C:A06 | chiron(cc187-NormBPHProstate) |
| 392 | 1054813 | 3556.J14.GZ43_507064 | M00084647D:C05 | chiron(cc187-NormBPHProstate) |
| 393 | 1226862 | 3556.J16.GZ43_507096 | M00084651B:G10 | chiron(cc187-NormBPHProstate) |
| 394 | 1224422 | 3556.K04.GZ43_506905 | M00084630D:F09 | chiron(cc187-NormBPHProstate) |
| 395 | 529709 | 3556.K12.GZ43_507033 | M00084645B:A06 | chiron(cc187-NormBPHProstate) |
| 396 | 450882 | 3556.K13.GZ43_507049 | M00084646B:B03 | chiron(cc187-NormBPHProstate) |
| 397 | 1224039 | 3556.K17.GZ43_507113 | M00084652D:G11 | chiron(cc187-NormBPHProstate) |
| 398 | 1224598 | 3556.L08.GZ43_506970 | M00084638D:A05 | chiron(cc187-NormBPHProstate) |
| 399 | 398211 | 3556.L09.GZ43_506986 | M00084641B:F08 | chiron(cc187-NormBPHProstate) |
| 400 | 1245188 | 3556.L16.GZ43_507098 | M00084651C:H01 | chiron(cc187-NormBPHProstate) |
| 401 | 558081 | 3556.L23.GZ43_507210 | M00084666C:A06 | chiron(cc187-NormBPHProstate) |
| 402 | 1224379 | 3556.M02.GZ43_506875 | M00084619A:G10 | chiron(cc187-NormBPHProstate) |
| 403 | 733538 | 3556.M11.GZ43_507019 | M00084644A:H05 | chiron(cc187-NormBPHProstate) |
| 404 | 727396 | 3556.M23.GZ43_507211 | M00084664D:E05 | chiron(cc187-NormBPHProstate) |
| 405 | 16872 | 3556.N02.GZ43_506876 | M00084620B:F05 | chiron(cc187-NormBPHProstate) |
| 406 | 1139849 | 3556.N04.GZ43_506908 | M00084631D:G01 | chiron(cc187-NormBPHProstate) |
| 407 | 1122528 | 3556.N05.GZ43_506924 | M00084633A:H05 | chiron(cc187-NormBPHProstate) |
| 408 | 1077319 | 3556.N06.GZ43_506940 | M00084634C:H02 | chiron(cc187-NormBPHProstate) |
| 409 | 1116087 | 3556.N21.GZ43_507180 | M00084659C:G05 | chiron(cc187-NormBPHProstate) |
| 410 | 1198563 | 3556.O08.GZ43_506973 | M00084638A:E10 | chiron(cc187-NormBPHProstate) |
| 411 | 585099 | 3556.O13.GZ43_507053 | M00084646B:D07 | chiron(cc187-NormBPHProstate) |
| 412 | 650520 | 3556.P07.GZ43_506958 | M00084637B:E01 | chiron(cc187-NormBPHProstate) |
| 413 | 844957 | 3559.A04.GZ43_507279 | M00084673B:H11 | chiron(cc187-NormBPHProstate) |
| 414 | 1077033 | 3559.A20.GZ43_507535 | M00084702A:B08 | chiron(cc187-NormBPHProstate) |
| 415 | 1187174 | 3559.A24.GZ43_507599 | M00084667C:A03 | chiron(cc187-NormBPHProstate) |
| 416 | 402789 | 3559.B04.GZ43_507280 | M00084675A:E02 | chiron(cc187-NormBPHProstate) |
| 417 | 863768 | 3559.B06.GZ43_507312 | M00084681B:G11 | chiron(cc187-NormBPHProstate) |
| 418 | 650263 | 3559.B08.GZ43_507344 | M00084684C:D02 | chiron(cc187-NormBPHProstate) |
| 419 | 1054038 | 3559.B10.GZ43_507376 | M00084687A:A03 | chiron(cc187-NormBPHProstate) |
| 420 | 38 | 3559.B18.GZ43_507504 | M00084700A:C10 | chiron(cc187-NormBPHProstate) |
| 421 | 1227324 | 3559.C06.GZ43_507313 | M00084679D:G12 | chiron(cc187-NormBPHProstate) |
| 422 | 1250373 | 3559.D21.GZ43_507554 | M00084704A:C12 | chiron(cc187-NormBPHProstate) |
| 423 | 1227912 | 3559.E06.GZ43_507315 | M00084680A:F08 | chiron(cc187-NormBPHProstate) |
| 424 | 1066654 | 3559.E09.GZ43_507363 | M00084685C:B12 | chiron(cc187-NormBPHProstate) |
| 425 | 703204 | 3559.E20.GZ43_507539 | M00084702B:C12 | chiron(cc187-NormBPHProstate) |
| 426 | 1227912 | 3559.F07.GZ43_507332 | M00084683B:A01 | chiron(cc187-NormBPHProstate) |
| 427 | 141870 | 3559.F17.GZ43_507492 | M00084699A:G05 | chiron(cc187-NormBPHProstate) |
| 428 | 15577 | 3559.H09.GZ43_507366 | M00084686B:B04 | chiron(cc187-NormBPHProstate) |
| 429 | 129786 | 3559.H22.GZ43_507574 | M00084705C:D01 | chiron(cc187-NormBPHProstate) |
| 430 | 454826 | 3559.H24.GZ43_507606 | M00084668D:D08 | chiron(cc187-NormBPHProstate) |
| 431 | 647587 | 3559.I05.GZ43_507303 | M00084676B:E02 | chiron(cc187-NormBPHProstate) |
| 432 | 915012 | 3559.J04.GZ43_507288 | M00084675B:A04 | chiron(cc187-NormBPHProstate) |
| 433 | 3155 | 3559.J20.GZ43_507544 | M00084703B:D09 | chiron(cc187-NormBPHProstate) |
| 434 | 1210953 | 3559.K16.GZ43_507481 | M00084696D:H04 | chiron(cc187-NormBPHProstate) |
| 435 | 576040 | 3559.K17.GZ43_507497 | M00084698B:D02 | chiron(cc187-NormBPHProstate) |
| 436 | 945247 | 3559.L01.GZ43_507242 | M00084670B:A09 | chiron(cc187-NormBPHProstate) |
| 437 | 1197444 | 3559.L14.GZ43_507450 | M00084694D:F04 | chiron(cc187-NormBPHProstate) |
| 438 | 573169 | 3559.L19.GZ43_507530 | M00084701C:E08 | chiron(cc187-NormBPHProstate) |
| 439 | 387256 | 3559.M02.GZ43_507259 | M00084671A:C12 | chiron(cc187-NormBPHProstate) |
| 440 | 1227993 | 3559.M09.GZ43_507371 | M00084685D:B11 | chiron(cc187-NormBPHProstate) |
| 441 | 1117392 | 3559.N05.GZ43_507308 | M00084678C:C11 | chiron(cc187-NormBPHProstate) |
| 442 | 726584 | 3559.N18.GZ43_507516 | M00084700D:E09 | chiron(cc187-NormBPHProstate) |
| 443 | 496962 | 3559.N21.GZ43_507564 | M00084704C:B09 | chiron(cc187-NormBPHProstate) |
| 444 | 402378 | 3559.O01.GZ43_507245 | M00084669C:A10 | chiron(cc187-NormBPHProstate) |
| 445 | 991366 | 3559.O05.GZ43_507309 | M00084677C:F03 | chiron(cc187-NormBPHProstate) |
| 446 | 860553 | 3559.O07.GZ43_507341 | M00084683A:B12 | chiron(cc187-NormBPHProstate) |
| 447 | 644687 | 3559.O20.GZ43_507549 | M00084703A:E04 | chiron(cc187-NormBPHProstate) |
| 448 | 129786 | 3559.P10.GZ43_507390 | M00084687C:F12 | chiron(cc187-NormBPHProstate) |
| 449 | 1062537 | 3559.P15.GZ43_507470 | M00084696C:A07 | chiron(cc187-NormBPHProstate) |
| 450 | 1197259 | 3559.P18.GZ43_507518 | M00084700D:H04 | chiron(cc187-NormBPHProstate) |
| 451 | 164618 | 3559.P24.GZ43_507614 | M00084669A:A05 | chiron(cc187-NormBPHProstate) |
| 452 | 1225264 | 3562.A01.GZ43_507615 | M00084707D:H03 | chiron(cc187-NormBPHProstate) |
| 453 | 1220708 | 3562.A15.GZ43_507839 | M00084727A:A02 | chiron(cc187-NormBPHProstate) |
| 454 | 727136 | 3562.B22.GZ43_507952 | M00084737A:C09 | chiron(cc187-NormBPHProstate) |
| 455 | 1225595 | 3562.C23.GZ43_507969 | M00084738B:A09 | chiron(cc187-NormBPHProstate) |
| 456 | 726522 | 3562.D10.GZ43_507762 | M00084720A:A01 | chiron(cc187-NormBPHProstate) |
| 457 | 846920 | 3562.E01.GZ43_507619 | M00084708A:A11 | chiron(cc187-NormBPHProstate) |
| 458 | 448356 | 3562.E03.GZ43_507651 | M00084710B:G07 | chiron(cc187-NormBPHProstate) |
| 459 | 1254733 | 3562.E12.GZ43_507795 | M00084722A:H12 | chiron(cc187-NormBPHProstate) |
| 460 | 453901 | 3562.F19.GZ43_507908 | M00084732B:A04 | chiron(cc187-NormBPHProstate) |
| 461 | 530453 | 3562.F20.GZ43_507924 | M00084734A:H01 | chiron(cc187-NormBPHProstate) |
| 462 | 1253670 | 3562.G13.GZ43_507813 | M00084723D:G09 | chiron(cc187-NormBPHProstate) |
| 463 | 971465 | 3562.G19.GZ43_507909 | M00084731C:G07 | chiron(cc187-NormBPHProstate) |
| 464 | 270 | 3562.H11.GZ43_507782 | M00084721C:F09 | chiron(cc187-NormBPHProstate) |
| 465 | 970165 | 3562.H12.GZ43_507798 | M00084722D:A03 | chiron(cc187-NormBPHProstate) |
| 466 | 726522 | 3562.I01.GZ43_507623 | M00084708B:A06 | chiron(cc187-NormBPHProstate) |
| 467 | 1053799 | 3562.I02.GZ43_507639 | M00084709C:B02 | chiron(cc187-NormBPHProstate) |
| 468 | 848701 | 3562.I13.GZ43_507815 | M00084724A:C02 | chiron(cc187-NormBPHProstate) |
| 469 | 1260846 | 3562.I15.GZ43_507847 | M00084727A:G09 | chiron(cc187-NormBPHProstate) |
| 470 | 1257096 | 3562.J09.GZ43_507752 | M00084718D:C04 | chiron(cc187-NormBPHProstate) |
| 471 | 1253087 | 3562.J13.GZ43_507816 | M00084724D:F04 | chiron(cc187-NormBPHProstate) |
| 472 | 447950 | 3562.K04.GZ43_507673 | M00084711B:A05 | chiron(cc187-NormBPHProstate) |
| 473 | 393948 | 3562.K08.GZ43_507737 | M00084716D:H03 | chiron(cc187-NormBPHProstate) |
| 474 | 893981 | 3562.L12.GZ43_507802 | M00084722D:G04 | chiron(cc187-NormBPHProstate) |
| 475 | 1224881 | 3562.N24.GZ43_507996 | M00084707D:B08 | chiron(cc187-NormBPHProstate) |
| 476 | 1141283 | 3562.O11.GZ43_507789 | M00084721B:C11 | chiron(cc187-NormBPHProstate) |
| 477 | 742101 | 3562.O18.GZ43_507901 | M00084730B:A09 | chiron(cc187-NormBPHProstate) |
| 478 | 34028 | 3562.O20.GZ43_507933 | M00084734A:E04 | chiron(cc187-NormBPHProstate) |
| 479 | 1254674 | 3562.P21.GZ43_507950 | M00084736B:H03 | chiron(cc187-NormBPHProstate) |
| 480 | 1226413 | 3562.P23.GZ43_507982 | M00084740C:B08 | chiron(cc187-NormBPHProstate) |
| 481 | 5375 | 3565.A23.GZ43_508351 | M00084742A:F07 | chiron(cc187-NormBPHProstate) |
| 482 | 628570 | 3565.B05.GZ43_508064 | M00084743A:E03 | chiron(cc187-NormBPHProstate) |
| 483 | 1055018 | 3565.B13.GZ43_508192 | M00084743D:G01 | chiron(cc187-NormBPHProstate) |
| 484 | 1139088 | 3565.B14.GZ43_508208 | M00084743D:H04 | chiron(cc187-NormBPHProstate) |
| 485 | 453522 | 3565.C04.GZ43_508049 | M00084745A:A08 | chiron(cc187-NormBPHProstate) |
| 486 | 551891 | 3565.C06.GZ43_508081 | M00084745A:H04 | chiron(cc187-NormBPHProstate) |
| 487 | 700354 | 3565.C17.GZ43_508257 | M00084746B:B04 | chiron(cc187-NormBPHProstate) |
| 488 | 1116087 | 3565.D14.GZ43_508210 | M00084747D:G02 | chiron(cc187-NormBPHProstate) |
| 489 | 640462 | 3565.D17.GZ43_508258 | M00084748A:D09 | chiron(cc187-NormBPHProstate) |
| 490 | 477110 | 3565.D19.GZ43_508290 | M00084748A:H02 | chiron(cc187-NormBPHProstate) |
| 491 | 34201 | 3565.E16.GZ43_508243 | M00084750C:B08 | chiron(cc187-NormBPHProstate) |
| 492 | 1225264 | 3565.G07.GZ43_508101 | M00084755A:D02 | chiron(cc187-NormBPHProstate) |
| 493 | 16872 | 3565.G09.GZ43_508133 | M00084755B:A04 | chiron(cc187-NormBPHProstate) |
| 494 | 1171518 | 3565.G22.GZ43_508341 | M00084755D:E06 | chiron(cc187-NormBPHProstate) |
| 495 | 1261580 | 3565.H06.GZ43_508086 | M00084756B:H01 | chiron(cc187-NormBPHProstate) |
| 496 | 1261892 | 3565.H10.GZ43_508150 | M00084756C:H01 | chiron(cc187-NormBPHProstate) |
| 497 | 454612 | 3565.H11.GZ43_508166 | M00084756D:C04 | chiron(cc187-NormBPHProstate) |
| 498 | 1258398 | 3565.H15.GZ43_508230 | M00084757A:D01 | chiron(cc187-NormBPHProstate) |
| 499 | 1259394 | 3565.H23.GZ43_508358 | M00084757B:F11 | chiron(cc187-NormBPHProstate) |
| 500 | 1225106 | 3565.H24.GZ43_508374 | M00084757B:F05 | chiron(cc187-NormBPHProstate) |
| 501 | 936795 | 3565.K15.GZ43_508233 | M00084760D:D09 | chiron(cc187-NormBPHProstate) |
| 502 | 552432 | 3565.L22.GZ43_508346 | M00084763D:A04 | chiron(cc187-NormBPHProstate) |
| 503 | 477692 | 3565.M15.GZ43_508235 | M00084764D:G08 | chiron(cc187-NormBPHProstate) |
| 504 | 1055063 | 3565.M20.GZ43_508315 | M00084765B:A10 | chiron(cc187-NormBPHProstate) |
| 505 | 1258456 | 3565.N12.GZ43_508188 | M00084766B:E03 | chiron(cc187-NormBPHProstate) |
| 506 | 1259319 | 3565.N13.GZ43_508204 | M00084766B:F02 | chiron(cc187-NormBPHProstate) |
| 507 | 1052399 | 3565.N19.GZ43_508300 | M00084766D:F12 | chiron(cc187-NormBPHProstate) |
| 508 | 1066041 | 3565.O02.GZ43_508029 | M00084767B:D10 | chiron(cc187-NormBPHProstate) |
| 509 | 1259872 | 3565.O03.GZ43_508045 | M00084767B:F06 | chiron(cc187-NormBPHProstate) |
| 510 | 1055029 | 3565.O07.GZ43_508109 | M00084767D:B04 | chiron(cc187-NormBPHProstate) |
| 511 | 1218793 | 3565.O15.GZ43_508237 | M00084768B:E09 | chiron(cc187-NormBPHProstate) |
| 512 | 141870 | 3565.P03.GZ43_508046 | M00084769C:H03 | chiron(cc187-NormBPHProstate) |
| 513 | 2424 | 3565.P09.GZ43_508142 | M00084770B:G12 | chiron(cc187-NormBPHProstate) |
| 514 | 477708 | 3565.P22.GZ43_508350 | M00084771D:A01 | chiron(cc187-NormBPHProstate) |
| 515 | 1226643 | 3565.P24.GZ43_508382 | M00084771D:G03 | chiron(cc187-NormBPHProstate) |
| 516 | 1224226 | 3568.A10.GZ43_508545 | M00084810D:B10 | chiron(cc187-NormBPHProstate) |
| 517 | 1171985 | 3568.B02.GZ43_508418 | M00084812A:C02 | chiron(cc187-NormBPHProstate) |
| 518 | 1193205 | 3568.B05.GZ43_508466 | M00084812A:E05 | chiron(cc187-NormBPHProstate) |
| 519 | 710194 | 3568.C22.GZ43_508739 | M00084817A:H11 | chiron(cc187-NormBPHProstate) |
| 520 | 1225335 | 3568.D23.GZ43_508756 | M00084820D:A03 | chiron(cc187-NormBPHProstate) |
| 521 | 402794 | 3568.E17.GZ43_508661 | M00084822B:G11 | chiron(cc187-NormBPHProstate) |
| 522 | 380634 | 3568.E20.GZ43_508709 | M00084822C:D06 | chiron(cc187-NormBPHProstate) |
| 523 | 389995 | 3568.F06.GZ43_508486 | M00084823A:H01 | chiron(cc187-NormBPHProstate) |
| 524 | 451390 | 3568.F07.GZ43_508502 | M00084823A:H06 | chiron(cc187-NormBPHProstate) |
| 525 | 1137259 | 3568.F11.GZ43_508566 | M00084823D:E05 | chiron(cc187-NormBPHProstate) |
| 526 | 1251950 | 3568.F12.GZ43_508582 | M00084823D:E06 | chiron(cc187-NormBPHProstate) |
| 527 | 1250995 | 3568.F22.GZ43_508742 | M00084824C:C10 | chiron(cc187-NormBPHProstate) |
| 528 | 1052466 | 3568.G10.GZ43_508551 | M00084826B:D12 | chiron(cc187-NormBPHProstate) |
| 529 | 621702 | 3568.G12.GZ43_508583 | M00084826B:E11 | chiron(cc187-NormBPHProstate) |
| 530 | 617292 | 3568.G24.GZ43_508775 | M00084827D:D04 | chiron(cc187-NormBPHProstate) |
| 531 | 8045 | 3568.H20.GZ43_508712 | M00084829B:F06 | chiron(cc187-NormBPHProstate) |
| 532 | 1224752 | 3568.J10.GZ43_508554 | M00084833A:G07 | chiron(cc187-NormBPHProstate) |
| 533 | 1210953 | 3568.J22.GZ43_508746 | M00084833D:B04 | chiron(cc187-NormBPHProstate) |
| 534 | 746389 | 3568.K01.GZ43_508411 | M00084834A:A03 | chiron(cc187-NormBPHProstate) |
| 535 | 845931 | 3568.K04.GZ43_508459 | M00084834B:G02 | chiron(cc187-NormBPHProstate) |
| 536 | 847516 | 3568.L04.GZ43_508460 | M00084835D:H03 | chiron(cc187-NormBPHProstate) |
| 537 | 1110143 | 3568.M03.GZ43_508445 | M00084837B:E06 | chiron(cc187-NormBPHProstate) |
| 538 | 1200453 | 3568.M13.GZ43_508605 | M00084838A:F12 | chiron(cc187-NormBPHProstate) |
| 539 | 1256602 | 3568.N11.GZ43_508574 | M00084839C:B09 | chiron(cc187-NormBPHProstate) |
| 540 | 1193970 | 3568.O17.GZ43_508671 | M00084841B:H09 | chiron(cc187-NormBPHProstate) |
| 541 | 1256564 | 3568.P04.GZ43_508464 | M00084842C:B07 | chiron(cc187-NormBPHProstate) |
| 542 | 1257732 | 3568.P18.GZ43_508688 | M00084843A:D06 | chiron(cc187-NormBPHProstate) |
| 543 | 7856 | 3568.P19.GZ43_508704 | M00084843A:G01 | chiron(cc187-NormBPHProstate) |
| 544 | 449697 | 3571.A04.GZ43_508833 | M00084843D:C06 | chiron(cc187-NormBPHProstate) |
| 545 | 1136972 | 3571.A07.GZ43_508881 | M00084843D:F05 | chiron(cc187-NormBPHProstate) |
| 546 | 1140865 | 3571.A08.GZ43_508897 | M00084844A:E10 | chiron(cc187-NormBPHProstate) |
| 547 | 1079863 | 3571.A11.GZ43_508945 | M00084844B:H08 | chiron(cc187-NormBPHProstate) |
| 548 | 1225500 | 3571.A14.GZ43_508993 | M00084844C:F04 | chiron(cc187-NormBPHProstate) |
| 549 | 1189027 | 3571.A22.GZ43_509121 | M00084845A:E02 | chiron(cc187-NormBPHProstate) |
| 550 | 1069632 | 3571.B13.GZ43_508978 | M00084845C:H05 | chiron(cc187-NormBPHProstate) |
| 551 | 1069632 | 3571.B22.GZ43_509122 | M00084846B:H07 | chiron(cc187-NormBPHProstate) |
| 552 | 387610 | 3571.C08.GZ43_508899 | M00084847B:G05 | chiron(cc187-NormBPHProstate) |
| 553 | 1261134 | 3571.D04.GZ43_508836 | M00084849A:H08 | chiron(cc187-NormBPHProstate) |
| 554 | 599820 | 3571.D07.GZ43_508884 | M00084849B:F11 | chiron(cc187-NormBPHProstate) |
| 555 | 418320 | 3571.E02.GZ43_508805 | M00084850C:A11 | chiron(cc187-NormBPHProstate) |
| 556 | 1224593 | 3571.E10.GZ43_508933 | M00084850D:H02 | chiron(cc187-NormBPHProstate) |
| 557 | 1116087 | 3571.E16.GZ43_509029 | M00084851C:F10 | chiron(cc187-NormBPHProstate) |
| 558 | 985859 | 3571.F06.GZ43_508870 | M00084853A:F08 | chiron(cc187-NormBPHProstate) |
| 559 | 1193236 | 3571.F16.GZ43_509030 | M00084853D:A12 | chiron(cc187-NormBPHProstate) |
| 560 | 504904 | 3571.F23.GZ43_509142 | M00084853D:G03 | chiron(cc187-NormBPHProstate) |
| 561 | 242901 | 3571.G22.GZ43_509127 | M00084855D:H05 | chiron(cc187-NormBPHProstate) |
| 562 | 1226845 | 3571.G24.GZ43_509159 | M00084856B:A12 | chiron(cc187-NormBPHProstate) |
| 563 | 140314 | 3571.H01.GZ43_508792 | M00084856B:D03 | chiron(cc187-NormBPHProstate) |
| 564 | 1260905 | 3571.H10.GZ43_508936 | M00084857A:G05 | chiron(cc187-NormBPHProstate) |
| 565 | 556772 | 3571.H12.GZ43_508968 | M00084857B:A09 | chiron(cc187-NormBPHProstate) |
| 566 | 1176182 | 3571.H16.GZ43_509032 | M00084857C:E11 | chiron(cc187-NormBPHProstate) |
| 567 | 1198072 | 3571.H18.GZ43_509064 | M00084857D:A11 | chiron(cc187-NormBPHProstate) |
| 568 | 677434 | 3571.I11.GZ43_508953 | M00084858C:B01 | chiron(cc187-NormBPHProstate) |
| 569 | 1193236 | 3571.J07.GZ43_508890 | M00084859C:D09 | chiron(cc187-NormBPHProstate) |
| 570 | 1055089 | 3571.J08.GZ43_508906 | M00084859C:H05 | chiron(cc187-NormBPHProstate) |
| 571 | 1054884 | 3571.J09.GZ43_508922 | M00084859D:B03 | chiron(cc187-NormBPHProstate) |
| 572 | 494499 | 3571.J14.GZ43_509002 | M00084860B:A01 | chiron(cc187-NormBPHProstate) |
| 573 | 686418 | 3571.L01.GZ43_508796 | M00084862B:B01 | chiron(cc187-NormBPHProstate) |
| 574 | 1140409 | 3571.M17.GZ43_509053 | M00084865D:B04 | chiron(cc187-NormBPHProstate) |
| 575 | 1260480 | 3571.M19.GZ43_509085 | M00084865D:G02 | chiron(cc187-NormBPHProstate) |
| 576 | 402516 | 3571.M24.GZ43_509165 | M00084866B:A03 | chiron(cc187-NormBPHProstate) |
| 577 | 1052374 | 3571.N09.GZ43_508926 | M00084866C:H04 | chiron(cc187-NormBPHProstate) |
| 578 | 1141620 | 3571.N14.GZ43_509006 | M00084867A:C11 | chiron(cc187-NormBPHProstate) |
| 579 | 1255330 | 3571.N17.GZ43_509054 | M00084867B:A03 | chiron(cc187-NormBPHProstate) |
| 580 | 1193699 | 3571.N22.GZ43_509134 | M00084867C:G02 | chiron(cc187-NormBPHProstate) |
| 581 | 1054884 | 3571.O08.GZ43_508911 | M00084868B:D01 | chiron(cc187-NormBPHProstate) |
| 582 | 866609 | 3574.A20.GZ43_509473 | M00084902B:A10 | chiron(cc187-NormBPHProstate) |
| 583 | 1059332 | 3574.B01.GZ43_509170 | M00084876D:A06 | chiron(cc187-NormBPHProstate) |
| 584 | 567700 | 3574.B04.GZ43_509218 | M00084880B:D03 | chiron(cc187-NormBPHProstate) |
| 585 | 1255268 | 3574.B10.GZ43_509314 | M00084888D:A11 | chiron(cc187-NormBPHProstate) |
| 586 | 401682 | 3574.B14.GZ43_509378 | M00084894A:G09 | chiron(cc187-NormBPHProstate) |
| 587 | 477110 | 3574.B24.GZ43_509538 | M00084874D:E03 | chiron(cc187-NormBPHProstate) |
| 588 | 554093 | 3574.C09.GZ43_509299 | M00084887C:C07 | chiron(cc187-NormBPHProstate) |
| 589 | 1225044 | 3574.C10.GZ43_509315 | M00084888C:D12 | chiron(cc187-NormBPHProstate) |
| 590 | 202671 | 3574.C12.GZ43_509347 | M00084890B:E02 | chiron(cc187-NormBPHProstate) |
| 591 | 89833 | 3574.C14.GZ43_509379 | M00084893C:A12 | chiron(cc187-NormBPHProstate) |
| 592 | 1225804 | 3574.C16.GZ43_509411 | M00084896B:G10 | chiron(cc187-NormBPHProstate) |
| 593 | 1225044 | 3574.C23.GZ43_509523 | M00084907C:C01 | chiron(cc187-NormBPHProstate) |
| 594 | 556772 | 3574.D02.GZ43_509188 | M00084878B:B12 | chiron(cc187-NormBPHProstate) |
| 595 | 1193236 | 3574.D12.GZ43_509348 | M00084890D:F09 | chiron(cc187-NormBPHProstate) |
| 596 | 676665 | 3574.E02.GZ43_509189 | M00084877D:G07 | chiron(cc187-NormBPHProstate) |
| 597 | 555059 | 3574.E03.GZ43_509205 | M00084879A:A04 | chiron(cc187-NormBPHProstate) |
| 598 | 450882 | 3574.E14.GZ43_509381 | M00084893C:B01 | chiron(cc187-NormBPHProstate) |
| 599 | 1053799 | 3574.F10.GZ43_509318 | M00084889A:B07 | chiron(cc187-NormBPHProstate) |
| 600 | 402286 | 3574.F18.GZ43_509446 | M00084900C:A04 | chiron(cc187-NormBPHProstate) |
| 601 | 1052364 | 3574.F23.GZ43_509526 | M00084908C:F07 | chiron(cc187-NormBPHProstate) |
| 602 | 496233 | 3574.G07.GZ43_509271 | M00084884D:D03 | chiron(cc187-NormBPHProstate) |
| 603 | 1193473 | 3574.G11.GZ43_509335 | M00084889C:A04 | chiron(cc187-NormBPHProstate) |
| 604 | 1223271 | 3574.H07.GZ43_509272 | M00084885D:A12 | chiron(cc187-NormBPHProstate) |
| 605 | 494890 | 3574.I02.GZ43_509193 | M00084877D:H09 | chiron(cc187-NormBPHProstate) |
| 606 | 447151 | 3574.I07.GZ43_509273 | M00084885A:C01 | chiron(cc187-NormBPHProstate) |
| 607 | 1224009 | 3574.J11.GZ43_509338 | M00084889D:G06 | chiron(cc187-NormBPHProstate) |
| 608 | 1224226 | 3574.J14.GZ43_509386 | M00084894B:F11 | chiron(cc187-NormBPHProstate) |
| 609 | 1054979 | 3574.J23.GZ43_509530 | M00084908D:B11 | chiron(cc187-NormBPHProstate) |
| 610 | 1225522 | 3574.K12.GZ43_509355 | M00084890C:A06 | chiron(cc187-NormBPHProstate) |
| 611 | 1226413 | 3574.K20.GZ43_509483 | M00084902C:F05 | chiron(cc187-NormBPHProstate) |
| 612 | 1223705 | 3574.L07.GZ43_509276 | M00084886A:C06 | chiron(cc187-NormBPHProstate) |
| 613 | 1258646 | 3574.M03.GZ43_509213 | M00084879B:E01 | chiron(cc187-NormBPHProstate) |
| 614 | 842459 | 3574.M23.GZ43_509533 | M00084908A:F03 | chiron(cc187-NormBPHProstate) |
| 615 | 1255745 | 3574.N04.GZ43_509230 | M00084880D:A10 | chiron(cc187-NormBPHProstate) |
| 616 | 652651 | 3574.N10.GZ43_509326 | M00084889B:C02 | chiron(cc187-NormBPHProstate) |
| 617 | 650715 | 3574.N12.GZ43_509358 | M00084891D:A02 | chiron(cc187-NormBPHProstate) |
| 618 | 628570 | 3574.N20.GZ43_509486 | M00084904A:D03 | chiron(cc187-NormBPHProstate) |
| 619 | 1053121 | 3574.P07.GZ43_509280 | M00084886B:D06 | chiron(cc187-NormBPHProstate) |
| 620 | 1142006 | 3574.P17.GZ43_509440 | M00084899D:B01 | chiron(cc187-NormBPHProstate) |
| 621 | 1226862 | 3577.A06.GZ43_509633 | M00084909C:G02 | chiron(cc187-NormBPHProstate) |
| 622 | 945247 | 3577.A18.GZ43_509825 | M00084910D:E07 | chiron(cc187-NormBPHProstate) |
| 623 | 915012 | 3577.B12.GZ43_509730 | M00084912D:A09 | chiron(cc187-NormBPHProstate) |
| 624 | 446747 | 3577.B15.GZ43_509778 | M00084912D:G06 | chiron(cc187-NormBPHProstate) |
| 625 | 819917 | 3577.B19.GZ43_509842 | M00084913B:F05 | chiron(cc187-NormBPHProstate) |
| 626 | 1224547 | 3577.E19.GZ43_509845 | M00084919C:B04 | chiron(cc187-NormBPHProstate) |
| 627 | 1249547 | 3577.F02.GZ43_509574 | M00084919D:B08 | chiron(cc187-NormBPHProstate) |
| 628 | 357367 | 3577.G07.GZ43_509655 | M00084921C:E04 | chiron(cc187-NormBPHProstate) |
| 629 | 725438 | 3577.G13.GZ43_509751 | M00084922A:C08 | chiron(cc187-NormBPHProstate) |
| 630 | 1193819 | 3577.H06.GZ43_509640 | M00084923D:B05 | chiron(cc187-NormBPHProstate) |
| 631 | 1224378 | 3577.H08.GZ43_509672 | M00084923D:F11 | chiron(cc187-NormBPHProstate) |
| 632 | 93073 | 3577.H18.GZ43_509832 | M00084925A:B08 | chiron(cc187-NormBPHProstate) |
| 633 | 737882 | 3577.I01.GZ43_509561 | M00084925C:G01 | chiron(cc187-NormBPHProstate) |
| 634 | 453958 | 3577.I17.GZ43_509817 | M00084926B:C05 | chiron(cc187-NormBPHProstate) |
| 635 | 1076930 | 3577.J04.GZ43_509610 | M00084927A:C01 | chiron(cc187-NormBPHProstate) |
| 636 | 1259955 | 3577.K06.GZ43_509643 | M00084928D:F06 | chiron(cc187-NormBPHProstate) |
| 637 | 1195403 | 3577.K14.GZ43_509771 | M00084929C:B02 | chiron(cc187-NormBPHProstate) |
| 638 | 1224978 | 3577.K23.GZ43_509915 | M00084930B:E12 | chiron(cc187-NormBPHProstate) |
| 639 | 1249731 | 3577.L10.GZ43_509708 | M00084930D:B08 | chiron(cc187-NormBPHProstate) |
| 640 | 52939 | 3577.N10.GZ43_509710 | M00084935B:E10 | chiron(cc187-NormBPHProstate) |
| 641 | 726922 | 3577.N14.GZ43_509774 | M00084935C:E07 | chiron(cc187-NormBPHProstate) |
| 642 | 552142 | 3577.O17.GZ43_509823 | M00084937D:B04 | chiron(cc187-NormBPHProstate) |
| 643 | 1140839 | 3577.O22.GZ43_509903 | M00084938B:A11 | chiron(cc187-NormBPHProstate) |
| 644 | 1222709 | 3577.P02.GZ43_509584 | M00084938B:F12 | chiron(cc187-NormBPHProstate) |
| 645 | 640277 | 3577.P07.GZ43_509664 | M00084938C:G06 | chiron(cc187-NormBPHProstate) |
| 646 | 654848 | 3577.P23.GZ43_509920 | M00084940B:F06 | chiron(cc187-NormBPHProstate) |
| 647 | 1225457 | 3580.A04.GZ43_509985 | M00084941B:E07 | chiron(cc187-NormBPHProstate) |
| 648 | 525759 | 3580.A09.GZ43_510065 | M00084941C:H04 | chiron(cc187-NormBPHProstate) |
| 649 | 160112 | 3580.A13.GZ43_510129 | M00084941D:C10 | chiron(cc187-NormBPHProstate) |
| 650 | 1139522 | 3580.A14.GZ43_510145 | M00084941D:H02 | chiron(cc187-NormBPHProstate) |
| 651 | 1036845 | 3580.B01.GZ43_509938 | M00084942C:B10 | chiron(cc187-NormBPHProstate) |
| 652 | 1223907 | 3580.C01.GZ43_509939 | M00084944D:E05 | chiron(cc187-NormBPHProstate) |
| 653 | 640157 | 3580.C03.GZ43_509971 | M00084945A:D10 | chiron(cc187-NormBPHProstate) |
| 654 | 1132413 | 3580.C05.GZ43_510003 | M00084945A:H10 | chiron(cc187-NormBPHProstate) |
| 655 | 621702 | 3580.D07.GZ43_510036 | M00084946D:H05 | chiron(cc187-NormBPHProstate) |
| 656 | 1227862 | 3580.D22.GZ43_510276 | M00084948B:F04 | chiron(cc187-NormBPHProstate) |
| 657 | 991366 | 3580.E02.GZ43_509957 | M00084948D:B08 | chiron(cc187-NormBPHProstate) |
| 658 | 439297 | 3580.E08.GZ43_510053 | M00084949B:B12 | chiron(cc187-NormBPHProstate) |
| 659 | 416687 | 3580.E10.GZ43_510085 | M00084949B:H11 | chiron(cc187-NormBPHProstate) |
| 660 | 1074909 | 3580.E19.GZ43_510229 | M00084950D:A06 | chiron(cc187-NormBPHProstate) |
| 661 | 1227862 | 3580.E21.GZ43_510261 | M00084950D:F05 | chiron(cc187-NormBPHProstate) |
| 662 | 566548 | 3580.E23.GZ43_510293 | M00084951A:D04 | chiron(cc187-NormBPHProstate) |
| 663 | 404121 | 3580.G03.GZ43_509975 | M00084953D:D03 | chiron(cc187-NormBPHProstate) |
| 664 | 828695 | 3580.G13.GZ43_510135 | M00084954C:A03 | chiron(cc187-NormBPHProstate) |
| 665 | 617629 | 3580.G14.GZ43_510151 | M00084954C:B12 | chiron(cc187-NormBPHProstate) |
| 666 | 453657 | 3580.G18.GZ43_510215 | M00084954D:A05 | chiron(cc187-NormBPHProstate) |
| 667 | 548217 | 3580.G19.GZ43_510231 | M00084954D:D12 | chiron(cc187-NormBPHProstate) |
| 668 | 453582 | 3580.G20.GZ43_510247 | M00084954D:E01 | chiron(cc187-NormBPHProstate) |
| 669 | 1139883 | 3580.G24.GZ43_510311 | M00084955A:E08 | chiron(cc187-NormBPHProstate) |
| 670 | 1122528 | 3580.H12.GZ43_510120 | M00084956B:B05 | chiron(cc187-NormBPHProstate) |
| 671 | 168225 | 3580.H16.GZ43_510184 | M00084956C:G09 | chiron(cc187-NormBPHProstate) |
| 672 | 1074160 | 3580.H22.GZ43_510280 | M00084957B:H07 | chiron(cc187-NormBPHProstate) |
| 673 | 1106730 | 3580.I06.GZ43_510025 | M00084958B:E10 | chiron(cc187-NormBPHProstate) |
| 674 | 1068036 | 3580.I08.GZ43_510057 | M00084958C:B03 | chiron(cc187-NormBPHProstate) |
| 675 | 1141753 | 3580.I18.GZ43_510217 | M00084959B:C07 | chiron(cc187-NormBPHProstate) |
| 676 | 649117 | 3580.J10.GZ43_510090 | M00084960D:D02 | chiron(cc187-NormBPHProstate) |
| 677 | 1227913 | 3580.J12.GZ43_510122 | M00084961A:C07 | chiron(cc187-NormBPHProstate) |
| 678 | 399738 | 3580.J18.GZ43_510218 | M00084961C:A06 | chiron(cc187-NormBPHProstate) |
| 679 | 501556 | 3580.J20.GZ43_510250 | M00084961C:F01 | chiron(cc187-NormBPHProstate) |
| 680 | 17075 | 3580.J21.GZ43_510266 | M00084961D:H03 | chiron(cc187-NormBPHProstate) |
| 681 | 532062 | 3580.K03.GZ43_509979 | M00084962C:F10 | chiron(cc187-NormBPHProstate) |
| 682 | 408711 | 3580.K05.GZ43_510011 | M00084963D:D07 | chiron(cc187-NormBPHProstate) |
| 683 | 640696 | 3580.K21.GZ43_510267 | M00084966A:A08 | chiron(cc187-NormBPHProstate) |
| 684 | 1136972 | 3580.L09.GZ43_510076 | M00084967B:B10 | chiron(cc187-NormBPHProstate) |
| 685 | 849333 | 3580.L10.GZ43_510092 | M00084967B:D09 | chiron(cc187-NormBPHProstate) |
| 686 | 1222709 | 3580.L12.GZ43_510124 | M00084967C:D10 | chiron(cc187-NormBPHProstate) |
| 687 | 738525 | 3580.L13.GZ43_510140 | M00084967C:D12 | chiron(cc187-NormBPHProstate) |
| 688 | 1053121 | 3580.L17.GZ43_510204 | M00084968A:D01 | chiron(cc187-NormBPHProstate) |
| 689 | 1226323 | 3580.M01.GZ43_509949 | M00084968C:D10 | chiron(cc187-NormBPHProstate) |
| 690 | 707590 | 3580.M16.GZ43_510189 | M00084969C:F03 | chiron(cc187-NormBPHProstate) |
| 691 | 376659 | 3580.M17.GZ43_510205 | M00084969C:H11 | chiron(cc187-NormBPHProstate) |
| 692 | 637915 | 3580.M18.GZ43_510221 | M00084969D:C11 | chiron(cc187-NormBPHProstate) |
| 693 | 1059445 | 3580.M23.GZ43_510301 | M00084970A:C11 | chiron(cc187-NormBPHProstate) |
| 694 | 1138419 | 3580.N10.GZ43_510094 | M00084970C:G03 | chiron(cc187-NormBPHProstate) |
| 695 | 1226494 | 3580.N11.GZ43_510110 | M00084970C:H09 | chiron(cc187-NormBPHProstate) |
| 696 | 427904 | 3580.N14.GZ43_510158 | M00084970D:B01 | chiron(cc187-NormBPHProstate) |
| 697 | 450882 | 3580.N15.GZ43_510174 | M00084970D:E08 | chiron(cc187-NormBPHProstate) |
| 698 | 961757 | 3580.N23.GZ43_510302 | M00084971C:G07 | chiron(cc187-NormBPHProstate) |
| 699 | 1060021 | 3580.O02.GZ43_509967 | M00084972B:H03 | chiron(cc187-NormBPHProstate) |
| 700 | 378629 | 3580.O06.GZ43_510031 | M00084973A:A01 | chiron(cc187-NormBPHProstate) |
| 701 | 12420 | 3580.O07.GZ43_510047 | M00084973A:B06 | chiron(cc187-NormBPHProstate) |
| 702 | 1138997 | 3580.O08.GZ43_510063 | M00084973A:C01 | chiron(cc187-NormBPHProstate) |
| 703 | 843831 | 3580.P04.GZ43_510000 | M00084974D:F11 | chiron(cc187-NormBPHProstate) |
| 704 | 862823 | 3580.P05.GZ43_510016 | M00084975A:G05 | chiron(cc187-NormBPHProstate) |
| 705 | 836879 | 3580.P14.GZ43_510160 | M00084976B:A08 | chiron(cc187-NormBPHProstate) |
| 706 | 1226267 | 3580.P19.GZ43_510240 | M00084976C:C12 | chiron(cc187-NormBPHProstate) |
| 707 | 725438 | 3583.B06.GZ43_510402 | M00084980C:B07 | chiron(cc187-NormBPHProstate) |
| 708 | 613411 | 3583.B07.GZ43_510418 | M00084980C:E06 | chiron(cc187-NormBPHProstate) |
| 709 | 861025 | 3583.B10.GZ43_510466 | M00084980D:D02 | chiron(cc187-NormBPHProstate) |
| 710 | 557717 | 3583.B11.GZ43_510482 | M00084980D:H08 | chiron(cc187-NormBPHProstate) |
| 711 | 400407 | 3583.D15.GZ43_510548 | M00084987A:D09 | chiron(cc187-NormBPHProstate) |
| 712 | 1193454 | 3583.D22.GZ43_510660 | M00084987B:H12 | chiron(cc187-NormBPHProstate) |
| 713 | 1064975 | 3583.E11.GZ43_510485 | M00084988B:B08 | chiron(cc187-NormBPHProstate) |
| 714 | 777670 | 3583.E13.GZ43_510517 | M00084988C:A04 | chiron(cc187-NormBPHProstate) |
| 715 | 1085645 | 3583.E15.GZ43_510549 | M00084988C:B01 | chiron(cc187-NormBPHProstate) |
| 716 | 509673 | 3583.E17.GZ43_510581 | M00084988C:G03 | chiron(cc187-NormBPHProstate) |
| 717 | 1118651 | 3583.F24.GZ43_510694 | M00084992D:B02 | chiron(cc187-NormBPHProstate) |
| 718 | 1119139 | 3583.G09.GZ43_510455 | M00084994A:H04 | chiron(cc187-NormBPHProstate) |
| 719 | 708025 | 3583.G16.GZ43_510567 | M00084994D:F11 | chiron(cc187-NormBPHProstate) |
| 720 | 698307 | 3583.G17.GZ43_510583 | M00084994D:H04 | chiron(cc187-NormBPHProstate) |
| 721 | 537118 | 3583.G21.GZ43_510647 | M00084995B:B08 | chiron(cc187-NormBPHProstate) |
| 722 | 447731 | 3583.H03.GZ43_510360 | M00084996B:D08 | chiron(cc187-NormBPHProstate) |
| 723 | 1226484 | 3583.H12.GZ43_510504 | M00084997D:H09 | chiron(cc187-NormBPHProstate) |
| 724 | 1059968 | 3583.H13.GZ43_510520 | M00084998A:C12 | chiron(cc187-NormBPHProstate) |
| 725 | 494499 | 3583.H15.GZ43_510552 | M00084998B:A04 | chiron(cc187-NormBPHProstate) |
| 726 | 496962 | 3583.J02.GZ43_510346 | M00085003C:D03 | chiron(cc187-NormBPHProstate) |
| 727 | 492489 | 3583.K08.GZ43_510443 | M00085006C:C07 | chiron(cc187-NormBPHProstate) |
| 728 | 1116503 | 3583.K10.GZ43_510475 | M00085006D:C10 | chiron(cc187-NormBPHProstate) |
| 729 | 552036 | 3583.K11.GZ43_510491 | M00085006D:C04 | chiron(cc187-NormBPHProstate) |
| 730 | 501556 | 3583.K14.GZ43_510539 | M00085007A:B03 | chiron(cc187-NormBPHProstate) |
| 731 | 164618 | 3583.K17.GZ43_510587 | M00085007B:C07 | chiron(cc187-NormBPHProstate) |
| 732 | 1274759 | 3583.K23.GZ43_510683 | M00085008B:H11 | chiron(cc187-NormBPHProstate) |
| 733 | 404308 | 3583.L05.GZ43_510396 | M00085009B:F10 | chiron(cc187-NormBPHProstate) |
| 734 | 234586 | 3583.L08.GZ43_510444 | M00085009C:C01 | chiron(cc187-NormBPHProstate) |
| 735 | 649117 | 3583.L09.GZ43_510460 | M00085009D:A02 | chiron(cc187-NormBPHProstate) |
| 736 | 353528 | 3583.L17.GZ43_510588 | M00085010C:H01 | chiron(cc187-NormBPHProstate) |
| 737 | 94413 | 3583.L21.GZ43_510652 | M00085011B:A01 | chiron(cc187-NormBPHProstate) |
| 738 | 401424 | 3583.M08.GZ43_510445 | M00085012C:A08 | chiron(cc187-NormBPHProstate) |
| 739 | 416470 | 3583.M10.GZ43_510477 | M00085012C:D06 | chiron(cc187-NormBPHProstate) |
| 740 | 380205 | 3583.M13.GZ43_510525 | M00085013A:E06 | chiron(cc187-NormBPHProstate) |
| 741 | 1054069 | 3583.N09.GZ43_510462 | M00085015A:C09 | chiron(cc187-NormBPHProstate) |
| 742 | 836879 | 3583.O03.GZ43_510367 | M00085017C:A11 | chiron(cc187-NormBPHProstate) |
| 743 | 451505 | 3583.O11.GZ43_510495 | M00085018C:B09 | chiron(cc187-NormBPHProstate) |
| 744 | 747805 | 3583.O17.GZ43_510591 | M00085019C:D05 | chiron(cc187-NormBPHProstate) |
| 745 | 1110143 | 3583.P09.GZ43_510464 | M00085021C:F06 | chiron(cc187-NormBPHProstate) |
| 746 | 821536 | 3583.P19.GZ43_510624 | M00085022B:B03 | chiron(cc187-NormBPHProstate) |
| 747 | 234667 | 3583.P22.GZ43_510672 | M00085022B:F05 | chiron(cc187-NormBPHProstate) |
| 748 | 1269942 | 3590.A12.GZ43_512274 | M00085023D:E11 | chiron(cc187-NormBPHProstate) |
| 749 | 1224593 | 3590.B01.GZ43_512099 | M00085025A:D11 | chiron(cc187-NormBPHProstate) |
| 750 | 386612 | 3590.B16.GZ43_512339 | M00085026D:A01 | chiron(cc187-NormBPHProstate) |
| 751 | 1250581 | 3590.B21.GZ43_512419 | M00085027A:C02 | chiron(cc187-NormBPHProstate) |
| 752 | 557063 | 3590.C20.GZ43_512404 | M00085029A:C02 | chiron(cc187-NormBPHProstate) |
| 753 | 1136977 | 3590.D03.GZ43_512133 | M00085029D:E12 | chiron(cc187-NormBPHProstate) |
| 754 | 18225 | 3590.D19.GZ43_512389 | M00085031B:E03 | chiron(cc187-NormBPHProstate) |
| 755 | 727730 | 3590.D23.GZ43_512453 | M00085031C:D05 | chiron(cc187-NormBPHProstate) |
| 756 | 1224704 | 3590.E08.GZ43_512214 | M00085032C:F04 | chiron(cc187-NormBPHProstate) |
| 757 | 1250660 | 3590.E10.GZ43_512246 | M00085032D:C03 | chiron(cc187-NormBPHProstate) |
| 758 | 846920 | 3590.F01.GZ43_512103 | M00085034B:E11 | chiron(cc187-NormBPHProstate) |
| 759 | 965814 | 3590.F16.GZ43_512343 | M00085035B:C12 | chiron(cc187-NormBPHProstate) |
| 760 | 439297 | 3590.G01.GZ43_512104 | M00085035D:D09 | chiron(cc187-NormBPHProstate) |
| 761 | 746389 | 3590.G02.GZ43_512120 | M00085035D:E04 | chiron(cc187-NormBPHProstate) |
| 762 | 677434 | 3590.H04.GZ43_512153 | M00085038A:B10 | chiron(cc187-NormBPHProstate) |
| 763 | 1055089 | 3590.H06.GZ43_512185 | M00085038A:C06 | chiron(cc187-NormBPHProstate) |
| 764 | 772985 | 3590.H09.GZ43_512233 | M00085038D:D10 | chiron(cc187-NormBPHProstate) |
| 765 | 624971 | 3590.H12.GZ43_512281 | M00085039B:E09 | chiron(cc187-NormBPHProstate) |
| 766 | 689639 | 3590.H16.GZ43_512345 | M00085039D:F09 | chiron(cc187-NormBPHProstate) |
| 767 | 1226488 | 3590.I16.GZ43_512346 | M00085047A:H02 | chiron(cc187-NormBPHProstate) |
| 768 | 403488 | 3590.J01.GZ43_512107 | M00085047D:F03 | chiron(cc187-NormBPHProstate) |
| 769 | 915012 | 3590.J02.GZ43_512123 | M00085047D:F08 | chiron(cc187-NormBPHProstate) |
| 770 | 1014734 | 3590.J18.GZ43_512379 | M00085049B:E03 | chiron(cc187-NormBPHProstate) |
| 771 | 1798 | 3590.J21.GZ43_512427 | M00085050A:B06 | chiron(cc187-NormBPHProstate) |
| 772 | 611592 | 3590.J22.GZ43_512443 | M00085050A:E11 | chiron(cc187-NormBPHProstate) |
| 773 | 374853 | 3590.K06.GZ43_512188 | M00085051A:G03 | chiron(cc187-NormBPHProstate) |
| 774 | 594116 | 3590.K10.GZ43_512252 | M00085051C:A01 | chiron(cc187-NormBPHProstate) |
| 775 | 551891 | 3590.K19.GZ43_512396 | M00085052B:E04 | chiron(cc187-NormBPHProstate) |
| 776 | 389995 | 3590.L08.GZ43_512221 | M00085053C:D07 | chiron(cc187-NormBPHProstate) |
| 777 | 483918 | 3590.L10.GZ43_512253 | M00085053D:D04 | chiron(cc187-NormBPHProstate) |
| 778 | 411960 | 3590.M03.GZ43_512142 | M00085056A:G12 | chiron(cc187-NormBPHProstate) |
| 779 | 532062 | 3590.M04.GZ43_512158 | M00085056B:B06 | chiron(cc187-NormBPHProstate) |
| 780 | 942200 | 3590.M09.GZ43_512238 | M00085056D:B12 | chiron(cc187-NormBPHProstate) |
| 781 | 470698 | 3590.N04.GZ43_512159 | M00085058A:H02 | chiron(cc187-NormBPHProstate) |
| 782 | 1088402 | 3590.N19.GZ43_512399 | M00085059B:H11 | chiron(cc187-NormBPHProstate) |
| 783 | 1226304 | 3590.N21.GZ43_512431 | M00085059B:H07 | chiron(cc187-NormBPHProstate) |
| 784 | 869453 | 3590.O08.GZ43_512224 | M00085060B:C05 | chiron(cc187-NormBPHProstate) |
| 785 | 737502 | 3596.C02.GZ43_512500 | M00085121A:D10 | chiron(cc187-NormBPHProstate) |
| 786 | 404418 | 3596.C20.GZ43_512788 | M00085123A:E07 | chiron(cc187-NormBPHProstate) |
| 787 | 404418 | 3596.C22.GZ43_512820 | M00085123B:C04 | chiron(cc187-NormBPHProstate) |
| 788 | 555967 | 3596.D01.GZ43_512485 | M00085123C:G11 | chiron(cc187-NormBPHProstate) |
| 789 | 1184134 | 3596.D07.GZ43_512581 | M00085124A:G04 | chiron(cc187-NormBPHProstate) |
| 790 | 89833 | 3596.D09.GZ43_512613 | M00085124B:G05 | chiron(cc187-NormBPHProstate) |
| 791 | 494890 | 3596.D17.GZ43_512741 | M00085125C:H06 | chiron(cc187-NormBPHProstate) |
| 792 | 401166 | 3596.E08.GZ43_512598 | M00085127C:C03 | chiron(cc187-NormBPHProstate) |
| 793 | 645986 | 3596.E22.GZ43_512822 | M00085129B:C02 | chiron(cc187-NormBPHProstate) |
| 794 | 1225870 | 3596.F10.GZ43_512631 | M00085131D:A06 | chiron(cc187-NormBPHProstate) |
| 795 | 269829 | 3596.G13.GZ43_512680 | M00085141C:G06 | chiron(cc187-NormBPHProstate) |
| 796 | 722878 | 3596.H04.GZ43_512537 | M00085142D:F04 | chiron(cc187-NormBPHProstate) |
| 797 | 454612 | 3596.H10.GZ43_512633 | M00085143C:D05 | chiron(cc187-NormBPHProstate) |
| 798 | 642869 | 3596.H17.GZ43_512745 | M00085144B:C12 | chiron(cc187-NormBPHProstate) |
| 799 | 770834 | 3596.H22.GZ43_512825 | M00085144D:G03 | chiron(cc187-NormBPHProstate) |
| 800 | 1189027 | 3596.I06.GZ43_512570 | M00085145C:D02 | chiron(cc187-NormBPHProstate) |
| 801 | 1189027 | 3596.I16.GZ43_512730 | M00085146B:C01 | chiron(cc187-NormBPHProstate) |
| 802 | 1189618 | 3596.J04.GZ43_512539 | M00085147C:A04 | chiron(cc187-NormBPHProstate) |
| 803 | 777670 | 3596.J13.GZ43_512683 | M00085148B:H01 | chiron(cc187-NormBPHProstate) |
| 804 | 1226243 | 3596.K14.GZ43_512700 | M00085151A:B04 | chiron(cc187-NormBPHProstate) |
| 805 | 1226510 | 3596.K15.GZ43_512716 | M00085151A:H09 | chiron(cc187-NormBPHProstate) |
| 806 | 517007 | 3596.L01.GZ43_512493 | M00085152B:A06 | chiron(cc187-NormBPHProstate) |
| 807 | 1070898 | 3596.L08.GZ43_512605 | M00085155B:F10 | chiron(cc187-NormBPHProstate) |
| 808 | 1117392 | 3596.L13.GZ43_512685 | M00085156A:G04 | chiron(cc187-NormBPHProstate) |
| 809 | 1227356 | 3596.N02.GZ43_512511 | M00085164C:G05 | chiron(cc187-NormBPHProstate) |
| 810 | 1042918 | 3596.N12.GZ43_512671 | M00085166C:A08 | chiron(cc187-NormBPHProstate) |
| 811 | 552432 | 3596.N15.GZ43_512719 | M00085166D:C10 | chiron(cc187-NormBPHProstate) |
| 812 | 585099 | 3596.N16.GZ43_512735 | M00085167A:G02 | chiron(cc187-NormBPHProstate) |
| 813 | 222216 | 3596.N21.GZ43_512815 | M00085167C:D06 | chiron(cc187-NormBPHProstate) |
| 814 | 650520 | 3596.O10.GZ43_512640 | M00085168D:D04 | chiron(cc187-NormBPHProstate) |
| 815 | 557717 | 3596.O12.GZ43_512672 | M00085169A:H12 | chiron(cc187-NormBPHProstate) |
| 816 | 90 | 3596.P03.GZ43_512529 | M00085171D:F05 | chiron(cc187-NormBPHProstate) |
| 817 | 747805 | 3596.P04.GZ43_512545 | M00085172A:G05 | chiron(cc187-NormBPHProstate) |
| 818 | 459967 | 3596.P07.GZ43_512593 | M00085172C:F06 | chiron(cc187-NormBPHProstate) |
| 819 | 454692 | 3596.P08.GZ43_512609 | M00085173A:B07 | chiron(cc187-NormBPHProstate) |
| 820 | 12420 | 3596.P10.GZ43_512641 | M00085173B:A08 | chiron(cc187-NormBPHProstate) |
| 821 | 1129928 | 3596.P21.GZ43_512817 | M00085175B:A03 | chiron(cc187-NormBPHProstate) |
| 822 | 7944 | 3599.A04.GZ43_512914 | M00085176C:B11 | chiron(cc187-NormBPHProstate) |
| 823 | 637367 | 3599.A23.GZ43_513218 | M00085178D:F01 | chiron(cc187-NormBPHProstate) |
| 824 | 1227253 | 3599.B15.GZ43_513091 | M00085182B:E04 | chiron(cc187-NormBPHProstate) |
| 825 | 1053861 | 3599.B16.GZ43_513107 | M00085182B:H10 | chiron(cc187-NormBPHProstate) |
| 826 | 401354 | 3599.C03.GZ43_512900 | M00085184D:B08 | chiron(cc187-NormBPHProstate) |
| 827 | 1117392 | 3599.C17.GZ43_513124 | M00085187B:C11 | chiron(cc187-NormBPHProstate) |
| 828 | 1116992 | 3599.D03.GZ43_512901 | M00085190B:C09 | chiron(cc187-NormBPHProstate) |
| 829 | 832148 | 3599.D05.GZ43_512933 | M00085190B:H04 | chiron(cc187-NormBPHProstate) |
| 830 | 1081548 | 3599.D07.GZ43_512965 | M00085190C:D10 | chiron(cc187-NormBPHProstate) |
| 831 | 1138578 | 3599.D10.GZ43_513013 | M00085191A:B03 | chiron(cc187-NormBPHProstate) |
| 832 | 847395 | 3599.E01.GZ43_512870 | M00085194C:B12 | chiron(cc187-NormBPHProstate) |
| 833 | 11683 | 3599.E05.GZ43_512934 | M00085194D:F04 | chiron(cc187-NormBPHProstate) |
| 834 | 1227623 | 3599.F17.GZ43_513127 | M00085201C:C12 | chiron(cc187-NormBPHProstate) |
| 835 | 555967 | 3599.F24.GZ43_513239 | M00085203A:E06 | chiron(cc187-NormBPHProstate) |
| 836 | 1226814 | 3599.H05.GZ43_512937 | M00085209C:F11 | chiron(cc187-NormBPHProstate) |
| 837 | 1227846 | 3599.H23.GZ43_513225 | M00085214D:G01 | chiron(cc187-NormBPHProstate) |
| 838 | 1226751 | 3599.J11.GZ43_513035 | M00085220D:E06 | chiron(cc187-NormBPHProstate) |
| 839 | 90 | 3599.K02.GZ43_512892 | M00085222D:D07 | chiron(cc187-NormBPHProstate) |
| 840 | 89833 | 3599.K04.GZ43_512924 | M00085223A:G01 | chiron(cc187-NormBPHProstate) |
| 841 | 854573 | 3599.K23.GZ43_513228 | M00085226C:F08 | chiron(cc187-NormBPHProstate) |
| 842 | 1066041 | 3599.L04.GZ43_512925 | M00085228B:C10 | chiron(cc187-NormBPHProstate) |
| 843 | 42285 | 3599.L15.GZ43_513101 | M00085229B:C10 | chiron(cc187-NormBPHProstate) |
| 844 | 661802 | 3599.M04.GZ43_512926 | M00085230B:G08 | chiron(cc187-NormBPHProstate) |
| 845 | 552428 | 3599.M22.GZ43_513214 | M00085242A:C06 | chiron(cc187-NormBPHProstate) |
| 846 | 996888 | 3599.M24.GZ43_513246 | M00085243A:D07 | chiron(cc187-NormBPHProstate) |
| 847 | 555509 | 3599.N09.GZ43_513007 | M00085244C:D03 | chiron(cc187-NormBPHProstate) |
| 848 | 1080634 | 3599.N16.GZ43_513119 | M00085245C:D07 | chiron(cc187-NormBPHProstate) |
| 849 | 180092 | 3599.N20.GZ43_513183 | M00085245D:G07 | chiron(cc187-NormBPHProstate) |
| 850 | 657748 | 3599.N24.GZ43_513247 | M00085246B:G12 | chiron(cc187-NormBPHProstate) |
| 851 | 555726 | 3599.O06.GZ43_512960 | M00085247A:F05 | chiron(cc187-NormBPHProstate) |
| 852 | 1055029 | 3599.O17.GZ43_513136 | M00085248B:G12 | chiron(cc187-NormBPHProstate) |
| 853 | 427182 | 3599.P05.GZ43_512945 | M00085249C:C11 | chiron(cc187-NormBPHProstate) |
| 854 | 1228277 | 3602.A09.GZ43_513378 | M00085252B:H07 | chiron(cc187-NormBPHProstate) |
| 855 | 1142632 | 3602.B18.GZ43_513523 | M00085255B:F11 | chiron(cc187-NormBPHProstate) |
| 856 | 1108332 | 3602.B21.GZ43_513571 | M00085255D:B09 | chiron(cc187-NormBPHProstate) |
| 857 | 734568 | 3602.B22.GZ43_513587 | M00085255D:E12 | chiron(cc187-NormBPHProstate) |
| 858 | 1085638 | 3602.C24.GZ43_513620 | M00085259A:H05 | chiron(cc187-NormBPHProstate) |
| 859 | 1136721 | 3602.D06.GZ43_513333 | M00085259D:B06 | chiron(cc187-NormBPHProstate) |
| 860 | 1284683 | 3602.D11.GZ43_513413 | M00085260C:A10 | chiron(cc187-NormBPHProstate) |
| 861 | 935460 | 3602.E04.GZ43_513302 | M00085262A:A02 | chiron(cc187-NormBPHProstate) |
| 862 | 425824 | 3602.E06.GZ43_513334 | M00085262C:E04 | chiron(cc187-NormBPHProstate) |
| 863 | 430201 | 3602.E13.GZ43_513446 | M00085263C:F12 | chiron(cc187-NormBPHProstate) |
| 864 | 646935 | 3602.E21.GZ43_513574 | M00085264C:F04 | chiron(cc187-NormBPHProstate) |
| 865 | 1064963 | 3602.F12.GZ43_513431 | M00083691C:E12 | chiron(cc187- |
| | | | | ProstateCancer4 + 4) |
| 866 | 1129928 | 3602.G03.GZ43_513288 | M00083692C:D09 | chiron(cc187- |
| | | | | ProstateCancer4 + 4) |
| 867 | 585099 | 3602.G17.GZ43_513512 | M00083694C:F09 | chiron(cc187- |
| | | | | ProstateCancer4 + 4) |
| 868 | 1194085 | 3602.I07.GZ43_513354 | M00083698B:H01 | chiron(cc187- |
| | | | | ProstateCancer4 + 4) |
| 869 | 993 | 3602.I11.GZ43_513418 | M00083698D:E01 | chiron(cc187- |
| | | | | ProstateCancer4 + 4) |
| 870 | 3123 | 3602.I15.GZ43_513482 | M00083699B:C12 | chiron(cc187- |
| | | | | ProstateCancer4 + 4) |
| 871 | 1193689 | 3602.J13.GZ43_513451 | M00083701D:G09 | chiron(cc187- |
| | | | | ProstateCancer4 + 4) |
| 872 | 19777 | 3602.K03.GZ43_513292 | M00083704C:C04 | chiron(cc187- |
| | | | | ProstateCancer4 + 4) |
| 873 | 637367 | 3602.K06.GZ43_513340 | M00083706A:D02 | chiron(cc187- |
| | | | | ProstateCancer4 + 4) |
| 874 | 13709 | 3602.L20.GZ43_513565 | M00083710D:B09 | chiron(cc187- |
| | | | | ProstateCancer4 + 4) |
| 875 | 1131409 | 3602.N03.GZ43_513295 | M00083714C:F04 | chiron(cc187- |
| | | | | ProstateCancer4 + 4) |
| 876 | 454733 | 3602.N06.GZ43_513343 | M00083715A:B11 | chiron(cc187- |
| | | | | ProstateCancer4 + 4) |
| 877 | 454349 | 3605.A15.gz43_513858 | M00083722B:A07 | chiron(cc187- |
| | | | | ProstateCancer4 + 4) |
| 878 | 1250995 | 3605.C16.gz43_513876 | M00083726B:E07 | chiron(cc187- |
| | | | | ProstateCancer4 + 4) |
| 879 | 1132413 | 3605.E19.gz43_513926 | M00083729C:F10 | chiron(cc187- |
| | | | | ProstateCancer4 + 4) |
| 880 | 397313 | 3605.G13.gz43_513832 | M00083733B:D08 | chiron(cc187- |
| | | | | ProstateCancer4 + 4) |
| 881 | 1292281 | 3605.H10.gz43_513785 | M00083735C:H12 | chiron(cc187- |
| | | | | ProstateCancer4 + 4) |
| 882 | 1248861 | 3605.H21.gz43_513961 | M00083736A:D10 | chiron(cc187- |
| | | | | ProstateCancer4 + 4) |
| 883 | 1292262 | 3605.I19.gz43_513930 | M00083738C:D05 | chiron(cc187- |
| | | | | ProstateCancer4 + 4) |
| 884 | 496233 | 3605.J16.gz43_513883 | M00083740C:G01 | chiron(cc187- |
| | | | | ProstateCancer4 + 4) |
| 885 | 1053793 | 3605.K19.gz43_513932 | M00083745A:A10 | chiron(cc187- |
| | | | | ProstateCancer4 + 4) |
| 886 | 734568 | 3605.M17.gz43_513902 | M00083749D:B09 | chiron(cc187- |
| | | | | ProstateCancer4 + 4) |
| 887 | 396320 | 3605.N04.gz43_513695 | M00083750D:G12 | chiron(cc187- |
| | | | | ProstateCancer4 + 4) |
| 888 | 453522 | 3605.N09.gz43_513775 | M00085266B:C06 | chiron(cc187- |
| | | | | ProstateCancer4 + 4) |
| 889 | 418320 | 3605.N12.gz43_513823 | M00085266D:C09 | chiron(cc187- |
| | | | | ProstateCancer4 + 4) |
| 890 | 1053747 | 3605.N16.gz43_513887 | M00085267B:D06 | chiron(cc187- |
| | | | | ProstateCancer4 + 4) |
| 891 | 644687 | 3608.B06.gz43_514099 | M00085282C:F05 | chiron(cc187- |
| | | | | ProstateCancer4 + 4) |
| 892 | 845354 | 3608.B12.gz43_514195 | M00085293B:B05 | chiron(cc187- |
| | | | | ProstateCancer4 + 4) |
| 893 | 850335 | 3608.B24.gz43_514387 | M00085273A:A09 | chiron(cc187- |
| | | | | ProstateCancer4 + 4) |
| 894 | 842459 | 3608.C18.gz43_514292 | M00085301C:H11 | chiron(cc187- |
| | | | | ProstateCancer4 + 4) |
| 895 | 653817 | 3608.E17.gz43_514278 | M00085299B:G01 | chiron(cc187- |
| | | | | ProstateCancer4 + 4) |
| 896 | 257547 | 3608.E20.gz43_514326 | M00085304B:D11 | chiron(cc187- |
| | | | | ProstateCancer4 + 4) |
| 897 | 1227454 | 3608.F13.gz43_514215 | M00085294D:G03 | chiron(cc187- |
| | | | | ProstateCancer4 + 4) |
| 898 | 1053854 | 3608.G09.gz43_514152 | M00085287C:G08 | chiron(cc187- |
| | | | | ProstateCancer4 + 4) |
| 899 | 1253234 | 3608.H05.gz43_514089 | M00085280D:F06 | chiron(cc187- |
| | | | | ProstateCancer4 + 4) |
| 900 | 1255330 | 3608.H14.gz43_514233 | M00085296D:H10 | chiron(cc187- |
| | | | | ProstateCancer4 + 4) |
| 901 | 974635 | 3608.H18.gz43_514297 | M00085302C:B06 | chiron(cc187- |
| | | | | ProstateCancer4 + 4) |
| 902 | 872646 | 3608.J17.gz43_514283 | M00085301B:C10 | chiron(cc187- |
| | | | | ProstateCancer4 + 4) |
| 903 | 939445 | 3608.J24.gz43_514395 | M00085273B:F08 | chiron(cc187- |
| | | | | ProstateCancer4 + 4) |
| 904 | 775820 | 3608.K03.gz43_514060 | M00085277D:C11 | chiron(cc187- |
| | | | | ProstateCancer4 + 4) |
| 905 | 452477 | 3608.K14.gz43_514236 | M00085296C:C09 | chiron(cc187- |
| | | | | ProstateCancer4 + 4) |
| 906 | 654848 | 3608.L07.gz43_514125 | M00085284D:A09 | chiron(cc187- |
| | | | | ProstateCancer4 + 4) |
| 907 | 1225734 | 3608.L14.gz43_514237 | M00085297A:A11 | chiron(cc187- |
| | | | | ProstateCancer4 + 4) |
| 908 | 404197 | 3608.N09.gz43_514159 | M00085288C:C09 | chiron(cc187- |
| | | | | ProstateCancer4 + 4) |
| 909 | 866237 | 3608.N19.gz43_514319 | M00085304A:B11 | chiron(cc187- |
| | | | | ProstateCancer4 + 4) |
| 910 | 650263 | 3608.N20.gz43_514335 | M00085305B:F10 | chiron(cc187- |
| | | | | ProstateCancer4 + 4) |
| 911 | 1141371 | 3608.O04.gz43_514080 | M00085278D:F03 | chiron(cc187- |
| | | | | ProstateCancer4 + 4) |
| 912 | 1257583 | 3608.P22.gz43_514369 | M00085309A:D11 | chiron(cc187- |
| | | | | ProstateCancer4 + 4) |
| 913 | 1252475 | 3611.A17.gz43_514658 | M00085312C:B09 | chiron(cc187- |
| | | | | ProstateCancer4 + 4) |
| 914 | 386255 | 3611.B11.gz43_514563 | M00085314C:F01 | chiron(cc187- |
| | | | | ProstateCancer4 + 4) |
| 915 | 1079196 | 3611.B16.gz43_514643 | M00085315C:B03 | chiron(cc187- |
| | | | | ProstateCancer4 + 4) |
| 916 | 1258456 | 3611.C09.gz43_514532 | M00085317B:G09 | chiron(cc187- |
| | | | | ProstateCancer4 + 4) |
| 917 | 403488 | 3611.E07.gz43_514502 | M00085322D:G05 | chiron(cc187- |
| | | | | ProstateCancer4 + 4) |
| 918 | 857031 | 3611.E12.gz43_514582 | M00085323C:H07 | chiron(cc187- |
| | | | | ProstateCancer4 + 4) |
| 919 | 1292600 | 3611.E20.gz43_514710 | M00085324B:F10 | chiron(cc187- |
| | | | | ProstateCancer4 + 4) |
| 920 | 1226862 | 3611.F15.gz43_514631 | M00085326B:D08 | chiron(cc187- |
| | | | | ProstateCancer4 + 4) |
| 921 | 857031 | 3611.H10.gz43_514553 | M00085331C:C07 | chiron(cc187- |
| | | | | ProstateCancer4 + 4) |
| 922 | 1292298 | 3611.H22.gz43_514745 | M00085332D:B03 | chiron(cc187- |
| | | | | ProstateCancer4 + 4) |
| 923 | 1183079 | 3611.I04.gz43_514458 | M00085333B:B10 | chiron(cc187- |
| | | | | ProstateCancer4 + 4) |
| 924 | 1258011 | 3611.I13.gz43_514602 | M00085334A:D10 | chiron(cc187- |
| | | | | ProstateCancer4 + 4) |
| 925 | 677858 | 3611.J04.gz43_514459 | M00085335B:D09 | chiron(cc187- |
| | | | | ProstateCancer4 + 4) |
| 926 | 1053747 | 3611.J15.gz43_514635 | M00085336C:G09 | chiron(cc187- |
| | | | | ProstateCancer4 + 4) |
| 927 | 404589 | 3611.J17.gz43_514667 | M00085336D:B10 | chiron(cc187- |
| | | | | ProstateCancer4 + 4) |
| 928 | 727730 | 3611.J22.gz43_514747 | M00085337B:F08 | chiron(cc187- |
| | | | | ProstateCancer4 + 4) |
| 929 | 451819 | 3611.K01.gz43_514412 | M00085337C:E09 | chiron(cc187- |
| | | | | ProstateCancer4 + 4) |
| 930 | 551691 | 3611.K12.gz43_514588 | M00085339C:C04 | chiron(cc187- |
| | | | | ProstateCancer4 + 4) |
| 931 | 1079863 | 3611.L22.gz43_514749 | M00085341C:H08 | chiron(cc187- |
| | | | | ProstateCancer4 + 4) |
| 932 | 1053747 | 3611.M18.gz43_514686 | M00085344A:G08 | chiron(cc187- |
| | | | | ProstateCancer4 + 4) |
| 933 | 1255745 | 3611.M24.gz43_514782 | M00085344D:B07 | chiron(cc187- |
| | | | | ProstateCancer4 + 4) |
| 934 | 1254418 | 3611.N01.gz43_514415 | M00085344D:F01 | chiron(cc187- |
| | | | | ProstateCancer4 + 4) |
| 935 | 504038 | 3611.N09.gz43_514543 | M00085345B:C09 | chiron(cc187- |
| | | | | ProstateCancer4 + 4) |
| 936 | 974223 | 3611.O16.gz43_514656 | M00085349A:C08 | chiron(cc187- |
| | | | | ProstateCancer4 + 4) |
| 937 | 1223936 | 3611.P08.gz43_514529 | M00085350D:G05 | chiron(cc187- |
| | | | | ProstateCancer4 + 4) |
| 938 | 1292289 | 3614.C18.gz43_515060 | M00085358B:A04 | chiron(cc187- |
| | | | | ProstateCancer4 + 4) |
| 939 | 1292423 | 3614.D14.gz43_514997 | M00085360B:G03 | chiron(cc187- |
| | | | | ProstateCancer4 + 4) |
| 940 | 1255745 | 3614.D21.gz43_515109 | M00085361A:A09 | chiron(cc187- |
| | | | | ProstateCancer4 + 4) |
| 941 | 1258714 | 3614.E06.gz43_514870 | M00085361D:F06 | chiron(cc187- |
| | | | | ProstateCancer4 + 4) |
| 942 | 1258491 | 3614.F22.gz43_515127 | M00085365C:C09 | chiron(cc187- |
| | | | | ProstateCancer4 + 4) |
| 943 | 1079196 | 3614.G20.gz43_515096 | M00085367B:C02 | chiron(cc187- |
| | | | | ProstateCancer4 + 4) |
| 944 | 1257531 | 3614.H09.gz43_514921 | M00085368B:A02 | chiron(cc187- |
| | | | | ProstateCancer4 + 4) |
| 945 | 1055256 | 3614.H22.gz43_515129 | M00085369D:G04 | chiron(cc187- |
| | | | | ProstateCancer4 + 4) |
| 946 | 1292439 | 3614.J07.gz43_514891 | M00085373C:B06 | chiron(cc187- |
| | | | | ProstateCancer4 + 4) |
| 947 | 761460 | 3614.K22.gz43_515132 | M00085380B:E10 | chiron(cc187- |
| | | | | ProstateCancer4 + 4) |
| 948 | 832347 | 3614.L13.gz43_514989 | M00085381C:A05 | chiron(cc187- |
| | | | | ProstateCancer4 + 4) |
| 949 | 855428 | 3614.M08.gz43_514910 | M00085383C:C05 | chiron(cc187- |
| | | | | ProstateCancer4 + 4) |
| 950 | 766134 | 3614.O02.gz43_514816 | M00085389B:B05 | chiron(cc187- |
| | | | | ProstateCancer4 + 4) |
| 951 | 875978 | 3614.O07.gz43_514896 | M00085389C:H04 | chiron(cc187- |
| | | | | ProstateCancer4 + 4) |
| 952 | 1226535 | 3614.O16.gz43_515040 | M00085390D:A03 | chiron(cc187- |
| | | | | ProstateCancer4 + 4) |
| 953 | 1204782 | 3614.P11.gz43_514961 | M00085393A:D06 | chiron(cc187- |
| | | | | ProstateCancer4 + 4) |
| 954 | 1293972 | 3614.P16.gz43_515041 | M00085393D:F12 | chiron(cc187- |
| | | | | ProstateCancer4 + 4) |
| 955 | 849333 | 3617.B16.gz43_515411 | M00085434C:G06 | chiron(cc187- |
| | | | | ProstateCancer4 + 4) |
| 956 | 721489 | 3617.C21.gz43_515492 | M00085444C:C02 | chiron(cc187- |
| | | | | ProstateCancer4 + 4) |
| 957 | 397313 | 3617.F10.gz43_515319 | M00085419A:G09 | chiron(cc187- |
| | | | | ProstateCancer4 + 4) |
| 958 | 1292436 | 3617.H16.gz43_515417 | M00085435A:B11 | chiron(cc187- |
| | | | | ProstateCancer4 + 4) |
| 959 | 963880 | 3617.I01.gz43_515178 | M00085396B:G04 | chiron(cc187- |
| | | | | ProstateCancer4 + 4) |
| 960 | 1227972 | 3617.L16.gz43_515421 | M00085435B:C05 | chiron(cc187- |
| | | | | ProstateCancer4 + 4) |
| 961 | 875978 | 3617.L21.gz43_515501 | M00085446A:D04 | chiron(cc187- |
| | | | | ProstateCancer4 + 4) |
| 962 | 1064963 | 3617.M08.gz43_515294 | M00085415A:D12 | chiron(cc187- |
| | | | | ProstateCancer4 + 4) |
| 963 | 1224505 | 3617.M13.gz43_515374 | M00085428B:G02 | chiron(cc187- |
| | | | | ProstateCancer4 + 4) |
| 964 | 453901 | 3617.N05.gz43_515247 | M00085406A:F03 | chiron(cc187- |
| | | | | ProstateCancer4 + 4) |
| 965 | 1292423 | 3617.N10.gz43_515327 | M00085419C:H05 | chiron(cc187- |
| | | | | ProstateCancer4 + 4) |
| 966 | 1273844 | 3617.N14.gz43_515391 | M00085432A:H08 | chiron(cc187- |
| | | | | ProstateCancer4 + 4) |
| 967 | 1227623 | 3617.N19.gz43_515471 | M00085441D:H10 | chiron(cc187- |
| | | | | ProstateCancer4 + 4) |
| 968 | 1292262 | 3617.P11.gz43_515345 | M00085422D:D07 | chiron(cc187- |
| | | | | ProstateCancer4 + 4) |
| 969 | 852027 | 3617.P12.gz43_515361 | M00085427C:A04 | chiron(cc187- |
| | | | | ProstateCancer4 + 4) |
| 970 | 396320 | 3617.P13.gz43_515377 | M00085430C:E04 | chiron(cc187- |
| | | | | ProstateCancer4 + 4) |
| 971 | 1273844 | 3620.B03.gz43_515810 | M00085454A:G06 | chiron(cc187- |
| | | | | ProstateCancer4 + 4) |
| 972 | 386255 | 3620.B24.gz43_516146 | M00085449C:D04 | chiron(cc187- |
| | | | | ProstateCancer4 + 4) |
| 973 | 1061206 | 3620.E12.gz43_515957 | M00085471B:H09 | chiron(cc187- |
| | | | | ProstateCancer4 + 4) |
| 974 | 1227838 | 3620.E13.gz43_515973 | M00085473C:B02 | chiron(cc187- |
| | | | | ProstateCancer4 + 4) |
| 975 | 1138472 | 3620.E17.gz43_516037 | M00085503D:D05 | chiron(cc187- |
| | | | | ProstateCancer4 + 4) |
| 976 | 1292413 | 3620.E19.gz43_516069 | M00085510B:G12 | chiron(cc187- |
| | | | | ProstateCancer4 + 4) |
| 977 | 691512 | 3620.E23.gz43_516133 | M00085520C:D02 | chiron(cc187- |
| | | | | ProstateCancer4 + 4) |
| 978 | 503696 | 3620.E24.gz43_516149 | M00085449A:E02 | chiron(cc187- |
| | | | | ProstateCancer4 + 4) |
| 979 | 1082476 | 3620.G17.gz43_516039 | M00085503D:G05 | chiron(cc187- |
| | | | | ProstateCancer4 + 4) |
| 980 | 84466 | 3620.G23.gz43_516135 | M00085520D:B11 | chiron(cc187- |
| | | | | ProstateCancer4 + 4) |
| 981 | 1283437 | 3620.J18.gz43_516058 | M00085509A:A02 | chiron(cc187- |
| | | | | ProstateCancer4 + 4) |
| 982 | 855428 | 3620.K19.gz43_516075 | M00085510C:A07 | chiron(cc187- |
| | | | | ProstateCancer4 + 4) |
| 983 | 1076930 | 3620.K24.gz43_516155 | M00085449B:G12 | chiron(cc187- |
| | | | | ProstateCancer4 + 4) |
| 984 | 866421 | 3620.O23.gz43_516143 | M00085522C:E05 | chiron(cc187- |
| | | | | ProstateCancer4 + 4) |
| 985 | 1228147 | 3623.B07.gz43_516258 | M00085548C:D04 | chiron(cc187- |
| | | | | ProstateCancer4 + 4) |
| 986 | 376659 | 3623.E03.gz43_516197 | M00085533C:D11 | chiron(cc187- |
| | | | | ProstateCancer4 + 4) |
| 987 | 1136977 | 3623.E15.gz43_516389 | M00085569B:C09 | chiron(cc187- |
| | | | | ProstateCancer4 + 4) |
| 988 | 1228288 | 3623.F03.gz43_516198 | M00085534D:H09 | chiron(cc187- |
| | | | | ProstateCancer4 + 4) |
| 989 | 867297 | 3623.F20.gz43_516470 | M00085583A:E06 | chiron(cc187- |
| | | | | ProstateCancer4 + 4) |
| 990 | 1294229 | 3623.G14.gz43_516375 | M00085566A:D12 | chiron(cc187- |
| | | | | ProstateCancer4 + 4) |
| 991 | 99774 | 3623.H07.gz43_516264 | M00085548D:F01 | chiron(cc187- |
| | | | | ProstateCancer4 + 4) |
| 992 | 1258398 | 3623.H10.gz43_516312 | M00085555D:F08 | chiron(cc187- |
| | | | | ProstateCancer4 + 4) |
| 993 | 1253684 | 3623.H23.gz43_516520 | M00085590A:G06 | chiron(cc187- |
| | | | | ProstateCancer4 + 4) |
| 994 | 1250581 | 3623.I08.gz43_516281 | M00085549D:G03 | chiron(cc187- |
| | | | | ProstateCancer4 + 4) |
| 995 | 453582 | 3623.I11.gz43_516329 | M00085557B:B02 | chiron(cc187- |
| | | | | ProstateCancer4 + 4) |
| 996 | 1082338 | 3623.L05.gz43_516236 | M00085541C:F06 | chiron(cc187- |
| | | | | ProstateCancer4 + 4) |
| 997 | 738525 | 3623.L24.gz43_516540 | M00085528A:E02 | chiron(cc187- |
| | | | | ProstateCancer4 + 4) |
| 998 | 1106730 | 3623.M10.gz43_516317 | M00085555A:A06 | chiron(cc187- |
| | | | | ProstateCancer4 + 4) |
| 999 | 1088930 | 3623.N23.gz43_516526 | M00085590B:F11 | chiron(cc187- |
| | | | | ProstateCancer4 + 4) |
| 1000 | 1261580 | 3623.P22.gz43_516512 | M00085588B:G10 | chiron(cc187- |
| | | | | ProstateCancer4 + 4) |
| 1001 | 13541 | 3626.A10.gz43_516689 | M00085592A:G06 | chiron(cc187- |
| | | | | ProstateCancer4 + 4) |
| 1002 | 1227336 | 3626.C16.gz43_516787 | M00085597C:C03 | chiron(cc187- |
| | | | | ProstateCancer4 + 4) |
| 1003 | 513843 | 3626.E07.gz43_516645 | M00085600B:B03 | chiron(cc187- |
| | | | | ProstateCancer4 + 4) |
| 1004 | 959989 | 3626.F03.gz43_516582 | M00085603A:E01 | chiron(cc187- |
| | | | | ProstateCancer4 + 4) |
| 1005 | 1121157 | 3626.G01.gz43_516551 | M00085605A:D08 | chiron(cc187- |
| | | | | ProstateCancer4 + 4) |
| 1006 | 548277 | 3626.I20.gz43_516857 | M00085611B:D03 | chiron(cc187- |
| | | | | ProstateCancer4 + 4) |
| 1007 | 965633 | 3626.I23.gz43_516905 | M00085611C:D09 | chiron(cc187- |
| | | | | ProstateCancer4 + 4) |
| 1008 | 868581 | 3626.M13.gz43_516749 | M00085620B:D08 | chiron(cc187- |
| | | | | ProstateCancer4 + 4) |
| 1009 | 1070898 | 3626.M15.gz43_516781 | M00085620C:F05 | chiron(cc187- |
| | | | | ProstateCancer4 + 4) |
| 1010 | 1293972 | 3626.N07.gz43_516654 | M00085623B:G11 | chiron(cc187- |
| | | | | ProstateCancer4 + 4) |
| 1011 | 406291 | 3626.N24.gz43_516926 | M00085625B:F01 | chiron(cc187- |
| | | | | ProstateCancer4 + 4) |
| 1012 | 1224492 | 3626.O08.gz43_516671 | M00085626B:B11 | chiron(cc187- |
| | | | | ProstateCancer4 + 4) |
| 1013 | 372868 | 3626.P11.gz43_516720 | M00085628B:D03 | chiron(cc187- |
| | | | | ProstateCancer4 + 4) |
| 1014 | 1227781 | 3626.P14.gz43_516768 | M00085628C:D08 | chiron(cc187- |
| | | | | ProstateCancer4 + 4) |
| 1015 | 15577 | 3629.A16.gz43_517169 | M00085630B:B09 | chiron(cc187- |
| | | | | ProstateCancer4 + 4) |
| 1016 | 1292413 | 3629.B14.gz43_517138 | M00085632C:A06 | chiron(cc187- |
| | | | | ProstateCancer4 + 4) |
| 1017 | 1257583 | 3629.C14.gz43_517139 | M00085635C:H07 | chiron(cc187- |
| | | | | ProstateCancer4 + 4) |
| 1018 | 1292996 | 3629.E01.gz43_516933 | M00085640A:H11 | chiron(cc187- |
| | | | | ProstateCancer4 + 4) |
| 1019 | 1202570 | 3629.E20.gz43_517237 | M00085641C:G01 | chiron(cc187- |
| | | | | ProstateCancer4 + 4) |
| 1020 | 1259955 | 3629.F24.gz43_517302 | M00085643D:F06 | chiron(cc187- |
| | | | | ProstateCancer4 + 4) |
| 1021 | 517444 | 3629.H10.gz43_517080 | M00085647A:C08 | chiron(cc187- |
| | | | | ProstateCancer4 + 4) |
| 1022 | 599820 | 3629.H12.gz43_517112 | M00085647A:E04 | chiron(cc187- |
| | | | | ProstateCancer4 + 4) |
| 1023 | 1226229 | 3629.I11.gz43_517097 | M00085649B:A03 | chiron(cc187- |
| | | | | ProstateCancer4 + 4) |
| 1024 | 1226413 | 3629.I16.gz43_517177 | M00085649C:A12 | chiron(cc187- |
| | | | | ProstateCancer4 + 4) |
| 1025 | 1292423 | 3629.J03.gz43_516970 | M00085650D:E12 | chiron(cc187- |
| | | | | ProstateCancer4 + 4) |
| 1026 | 1106730 | 3629.J07.gz43_517034 | M00085651A:A01 | chiron(cc187- |
| | | | | ProstateCancer4 + 4) |
| 1027 | 966355 | 3632.C11.gz43_517475 | M00085676C:C04 | chiron(cc187- |
| | | | | ProstateCancer4 + 4) |
| 1028 | 28706 | 3632.C17.gz43_517571 | M00085677A:E02 | chiron(cc187- |
| | | | | ProstateCancer4 + 4) |
| 1029 | 402588 | 3632.F07.gz43_517414 | M00085683B:B10 | chiron(cc187- |
| | | | | ProstateCancer4 + 4) |
| 1030 | 1055326 | 3632.G01.gz43_517319 | M00085686A:C05 | chiron(cc187- |
| | | | | ProstateCancer4 + 4) |
| 1031 | 16472 | 3632.I20.gz43_517625 | M00085691A:E06 | chiron(cc187- |
| | | | | ProstateCancer4 + 4) |
| 1032 | 1082360 | 3632.K20.gz43_517627 | M00085694D:D06 | chiron(cc187- |
| | | | | ProstateCancer4 + 4) |
| 1033 | 1258491 | 3632.M08.gz43_517437 | M00085697A:F01 | chiron(cc187- |
| | | | | ProstateCancer4 + 4) |
| 1034 | 452564 | 3632.M13.gz43_517517 | M00085697B:G05 | chiron(cc187- |
| | | | | ProstateCancer4 + 4) |
| 1035 | 854573 | 3632.M19.gz43_517613 | M00085697D:H11 | chiron(cc187- |
| | | | | ProstateCancer4 + 4) |
| 1036 | 1292996 | 3632.N13.gz43_517518 | M00085701A:A09 | chiron(cc187- |
| | | | | ProstateCancer4 + 4) |
| 1037 | 1055256 | 3632.N21.gz43_517646 | M00085701D:A02 | chiron(cc187- |
| | | | | ProstateCancer4 + 4) |
| 1038 | 18225 | 3632.O06.gz43_517407 | M00085702B:G11 | chiron(cc187- |
| | | | | ProstateCancer4 + 4) |
| 1039 | 1053854 | 3632.P07.gz43_517424 | M00085705A:E01 | chiron(cc187- |
| | | | | ProstateCancer4 + 4) |
| 1040 | 1292582 | 3635.A06.gz43_517777 | M00085707A:A04 | chiron(cc187- |
| | | | | ProstateCancer4 + 4) |
| 1041 | 486961 | 3635.A08.gz43_517809 | M00085707A:F01 | chiron(cc187- |
| | | | | ProstateCancer4 + 4) |
| 1042 | 1171582 | 3635.A13.gz43_517889 | M00085707C:A10 | chiron(cc187- |
| | | | | ProstateCancer4 + 4) |
| 1043 | 652651 | 3635.D07.gz43_517796 | M00085714C:G03 | chiron(cc187- |
| | | | | ProstateCancer4 + 4) |
| 1044 | 1252170 | 3635.F01.gz43_517702 | M00085720D:A03 | chiron(cc187- |
| | | | | ProstateCancer4 + 4) |
| 1045 | 761460 | 3635.F06.gz43_517782 | M00085721A:G03 | chiron(cc187- |
| | | | | ProstateCancer4 + 4) |
| 1046 | 1082195 | 3635.F10.gz43_517846 | M00085722A:A06 | chiron(cc187- |
| | | | | ProstateCancer4 + 4) |
| 1047 | 523931 | 3635.H20.gz43_518008 | M00085728B:C08 | chiron(cc187- |
| | | | | ProstateCancer4 + 4) |
| 1048 | 731936 | 3635.J06.gz43_517786 | M00085732A:B09 | chiron(cc187- |
| | | | | ProstateCancer4 + 4) |
| 1049 | 1227838 | 3635.J09.gz43_517834 | M00085732A:G04 | chiron(cc187- |
| | | | | ProstateCancer4 + 4) |
| 1050 | 1258456 | 3635.K05.gz43_517771 | M00085733D:E05 | chiron(cc187- |
| | | | | ProstateCancer4 + 4) |
| 1051 | 1209252 | 3635.K06.gz43_517787 | M00085733D:F08 | chiron(cc187- |
| | | | | ProstateCancer4 + 4) |
| 1052 | 484459 | 3635.M18.gz43_517981 | M00085741C:D06 | chiron(cc187- |
| | | | | ProstateCancer4 + 4) |
| 1053 | 1053514 | 3635.O01.gz43_517711 | M00085745C:C03 | chiron(cc187- |
| | | | | ProstateCancer4 + 4) |
| 1054 | 458490 | 3635.O14.gz43_517919 | M00085747A:B11 | chiron(cc187- |
| | | | | ProstateCancer4 + 4) |
| 1055 | 1258620 | 3635.P17.gz43_517968 | M00085750A:G03 | chiron(cc187- |
| | | | | ProstateCancer4 + 4) |
| 1056 | 733840 | 3635.P18.gz43_517984 | M00085750B:C10 | chiron(cc187- |
| | | | | ProstateCancer4 + 4) |
| 1057 | 549024 | 3638.A02.gz43_518097 | M00085754C:A12 | chiron(cc187- |
| | | | | ProstateCancer4 + 4) |
| 1058 | 1248861 | 3638.A24.gz43_518449 | M00085751A:A11 | chiron(cc187- |
| | | | | ProstateCancer4 + 4) |
| 1059 | 1088847 | 3638.F15.gz43_518310 | M00085786D:G12 | chiron(cc187- |
| | | | | ProstateCancer4 + 4) |
| 1060 | 846056 | 3638.H07.gz43_518184 | M00085764B:H12 | chiron(cc187- |
| | | | | ProstateCancer4 + 4) |
| 1061 | 731936 | 3638.J09.gz43_518218 | M00085770C:A12 | chiron(cc187- |
| | | | | ProstateCancer4 + 4) |
| 1062 | 858745 | 3638.K06.gz43_518171 | M00085761C:E07 | chiron(cc187- |
| | | | | ProstateCancer4 + 4) |
| 1063 | 400760 | 3638.L10.gz43_518236 | M00085773A:F05 | chiron(cc187- |
| | | | | ProstateCancer4 + 4) |
| 1064 | 1292600 | 3638.N05.gz43_518158 | M00085761A:B03 | chiron(cc187- |
| | | | | ProstateCancer4 + 4) |
| 1065 | 557994 | 3643.D21.gz43_518788 | M00085882B:F11 | chiron(cc187- |
| | | | | ProstateCancer4 + 4) |
| 1066 | 1225500 | 3643.E24.gz43_518837 | M00085886C:F05 | chiron(cc187- |
| | | | | ProstateCancer4 + 4) |
| 1067 | 684638 | 3643.F07.gz43_518566 | M00085887C:B03 | chiron(cc187- |
| | | | | ProstateCancer4 + 4) |
| 1068 | 1230257 | 3643.G20.gz43_518775 | M00085891D:E07 | chiron(cc187- |
| | | | | ProstateCancer4 + 4) |
| 1069 | 448356 | 3643.G24.gz43_518839 | M00085892A:F04 | chiron(cc187- |
| | | | | ProstateCancer4 + 4) |
| 1070 | 18376 | 3643.H09.gz43_518600 | M00085893B:D08 | chiron(cc187- |
| | | | | ProstateCancer4 + 4) |
| 1071 | 676129 | 3643.I01.gz43_518473 | M00085896D:A11 | chiron(cc187- |
| | | | | ProstateCancer4 + 4) |
| 1072 | 44071 | 3643.I02.gz43_518489 | M00085896D:A09 | chiron(cc187- |
| | | | | ProstateCancer4 + 4) |
| 1073 | 1224505 | 3643.I18.gz43_518745 | M00085899C:G10 | chiron(cc187- |
| | | | | ProstateCancer4 + 4) |
| 1074 | 1292436 | 3643.I24.gz43_518841 | M00085900B:E02 | chiron(cc187- |
| | | | | ProstateCancer4 + 4) |
| 1075 | 1293040 | 3643.K06.gz43_518555 | M00085904D:D02 | chiron(cc187- |
| | | | | ProstateCancer4 + 4) |
| 1076 | 1271515 | 3643.L01.gz43_518476 | M00085907B:B11 | chiron(cc187- |
| | | | | ProstateCancer4 + 4) |
| 1077 | 683650 | 3643.N24.gz43_518846 | M00085917C:A04 | chiron(cc187- |
| | | | | ProstateCancer4 + 4) |
| 1078 | 684462 | 3643.O16.gz43_518719 | M00085918D:C11 | chiron(cc187- |
| | | | | ProstateCancer4 + 4) |
| 1079 | 5375 | 3643.O18.gz43_518751 | M00085919A:A05 | chiron(cc187- |
| | | | | ProstateCancer4 + 4) |
| 1080 | 1294075 | 3643.O21.gz43_518799 | M00085919B:F02 | chiron(cc187- |
| | | | | ProstateCancer4 + 4) |
| 1081 | 1271515 | 3643.P13.gz43_518672 | M00085922A:A08 | chiron(cc187- |
| | | | | ProstateCancer4 + 4) |
| 1082 | 558076 | 3643.P14.gz43_518688 | M00085922A:E10 | chiron(cc187- |
| | | | | ProstateCancer4 + 4) |
| 1083 | 1251950 | 3646.A07.gz43_518945 | M00085926C:C06 | chiron(cc187- |
| | | | | ProstateCancer4 + 4) |
| 1084 | 453767 | 3646.A09.gz43_518977 | M00085927A:F06 | chiron(cc187- |
| | | | | ProstateCancer4 + 4) |
| 1085 | 13541 | 3646.A12.gz43_519025 | M00085927C:G10 | chiron(cc187- |
| | | | | ProstateCancer4 + 4) |
| 1086 | 1230257 | 3646.A13.gz43_519041 | M00085927C:G11 | chiron(cc187- |
| | | | | ProstateCancer4 + 4) |
| 1087 | 1079196 | 3646.B20.gz43_519154 | M00085933B:B06 | chiron(cc187- |
| | | | | ProstateCancer4 + 4) |
| 1088 | 1061206 | 3646.C06.gz43_518931 | M00085934B:E12 | chiron(cc187- |
| | | | | ProstateCancer4 + 4) |
| 1089 | 726922 | 3646.C16.gz43_519091 | M00085935D:H04 | chiron(cc187- |
| | | | | ProstateCancer4 + 4) |
| 1090 | 508322 | 3646.E02.gz43_518869 | M00085941B:A06 | chiron(cc187- |
| | | | | ProstateCancer4 + 4) |
| 1091 | 167565 | 3646.E20.gz43_519157 | M00085944A:F04 | chiron(cc187- |
| | | | | ProstateCancer4 + 4) |
| 1092 | 1226588 | 3646.H04.gz43_518904 | M00085955C:C03 | chiron(cc187- |
| | | | | ProstateCancer4 + 4) |
| 1093 | 402424 | 3646.H09.gz43_518984 | M00085955D:H10 | chiron(cc187- |
| | | | | ProstateCancer4 + 4) |
| 1094 | 1182447 | 3646.H16.gz43_519096 | M00085956B:E08 | chiron(cc187- |
| | | | | ProstateCancer4 + 4) |
| 1095 | 1136803 | 3646.I01.gz43_518857 | M00085956D:G04 | chiron(cc187- |
| | | | | ProstateCancer4 + 4) |
| 1096 | 500798 | 3646.J03.gz43_518890 | M00085959B:D04 | chiron(cc187- |
| | | | | ProstateCancer4 + 4) |
| 1097 | 727449 | 3646.J22.gz43_519194 | M00085962B:A12 | chiron(cc187- |
| | | | | ProstateCancer4 + 4) |
| 1098 | 1292289 | 3646.K14.gz43_519067 | M00085964A:B11 | chiron(cc187- |
| | | | | ProstateCancer4 + 4) |
| 1099 | 1293972 | 3646.L17.gz43_519116 | M00085968B:F09 | chiron(cc187- |
| | | | | ProstateCancer4 + 4) |
| 1100 | 848701 | 3646.O13.gz43_519055 | M00085980A:G10 | chiron(cc187- |
| | | | | ProstateCancer4 + 4) |
| 1101 | 5375 | 3646.O16.gz43_519103 | M00085980B:F06 | chiron(cc187- |
| | | | | ProstateCancer4 + 4) |
| 1102 | 862845 | 3646.P09.gz43_518992 | M00085982D:C06 | chiron(cc187- |
| | | | | ProstateCancer4 + 4) |
| 1103 | 738525 | 3646.P14.gz43_519072 | M00085985C:D02 | chiron(cc187- |
| | | | | ProstateCancer4 + 4) |
| 1104 | 968647 | 3646.P17.gz43_519120 | M00085986B:H02 | chiron(cc187- |
| | | | | ProstateCancer4 + 4) |
| 1105 | 1292389 | 3661.A08.gz43_519483 | M00085988D:F09 | chiron(cc187- |
| | | | | ProstateCancer4 + 4) |
| 1106 | 1105945 | 3661.D17.gz43_519630 | M00086000A:C05 | chiron(cc187- |
| | | | | ProstateCancer4 + 4) |
| 1107 | 1257477 | 3661.D18.gz43_519646 | M00086000C:B08 | chiron(cc187- |
| | | | | ProstateCancer4 + 4) |
| 1108 | 1247030 | 3661.E19.gz43_519663 | M00086003D:B10 | chiron(cc187- |
| | | | | ProstateCancer4 + 4) |
| 1109 | 189355 | 3661.E23.gz43_519727 | M00086003D:G08 | chiron(cc187- |
| | | | | ProstateCancer4 + 4) |
| 1110 | 1129928 | 3661.F14.gz43_519584 | M00086005A:B02 | chiron(cc187- |
| | | | | ProstateCancer4 + 4) |
| 1111 | 1224881 | 3661.G16.gz43_519617 | M00086008D:F08 | chiron(cc187- |
| | | | | ProstateCancer4 + 4) |
| 1112 | 504038 | 3661.G20.gz43_519681 | M00086010B:B05 | chiron(cc187- |
| | | | | ProstateCancer4 + 4) |
| 1113 | 558081 | 3661.H11.gz43_519538 | M00086015A:B03 | chiron(cc187- |
| | | | | ProstateCancer4 + 4) |
| 1114 | 609914 | 3661.H24.gz43_519746 | M00086015D:G04 | chiron(cc187- |
| | | | | ProstateCancer4 + 4) |
| 1115 | 1292600 | 3661.I22.gz43_519715 | M00086018A:A05 | chiron(cc187- |
| | | | | ProstateCancer4 + 4) |
| 1116 | 1088847 | 3661.J15.gz43_519604 | M00086020C:E09 | chiron(cc187- |
| | | | | ProstateCancer4 + 4) |
| 1117 | 1292262 | 3661.K22.gz43_519717 | M00086027A:G04 | chiron(cc187- |
| | | | | ProstateCancer4 + 4) |
| 1118 | 19161 | 3661.L19.gz43_519670 | M00086031C:G11 | chiron(cc187- |
| | | | | ProstateCancer4 + 4) |
| 1119 | 380634 | 3661.M03.gz43_519415 | M00086035A:C11 | chiron(cc187- |
| | | | | ProstateCancer4 + 4) |
| 1120 | 1056246 | 3661.M23.gz43_519735 | M00086038A:D03 | chiron(cc187- |
| | | | | ProstateCancer4 + 4) |
| 1121 | 678054 | 3661.P22.gz43_519722 | M00086048D:H08 | chiron(cc187- |
| | | | | ProstateCancer4 + 4) |
| 1122 | 691512 | 3662.A13.gz43_519947 | M00086053B:F01 | chiron(cc187- |
| | | | | ProstateCancer4 + 4) |
| 1123 | 841016 | 3662.B13.gz43_519948 | M00086057A:F07 | chiron(cc187- |
| | | | | ProstateCancer4 + 4) |
| 1124 | 496233 | 3662.C10.gz43_519901 | M00086060C:F04 | chiron(cc187- |
| | | | | ProstateCancer4 + 4) |
| 1125 | 484459 | 3662.C15.gz43_519981 | M00086061B:E02 | chiron(cc187- |
| | | | | ProstateCancer4 + 4) |
| 1126 | 448177 | 3662.F13.gz43_519952 | M00086076B:D02 | chiron(cc187- |
| | | | | ProstateCancer4 + 4) |
| 1127 | 455461 | 3662.H14.gz43_519970 | M00086081B:A06 | chiron(cc187- |
| | | | | ProstateCancer4 + 4) |
| 1128 | 1253902 | 3662.H23.gz43_520114 | M00086081D:D09 | chiron(cc187- |
| | | | | ProstateCancer4 + 4) |
| 1129 | 517014 | 3662.H24.gz43_520130 | M00086081D:H11 | chiron(cc187- |
| | | | | ProstateCancer4 + 4) |
| 1130 | 1250660 | 3662.J05.gz43_519828 | M00086084D:E12 | chiron(cc187- |
| | | | | ProstateCancer4 + 4) |
| 1131 | 733840 | 3662.J08.gz43_519876 | M00086085A:G05 | chiron(cc187- |
| | | | | ProstateCancer4 + 4) |
| 1132 | 99774 | 3662.J09.gz43_519892 | M00086085A:H03 | chiron(cc187- |
| | | | | ProstateCancer4 + 4) |
| 1133 | 1253234 | 3662.J16.gz43_520004 | M00086085D:F09 | chiron(cc187- |
| | | | | ProstateCancer4 + 4) |
| 1134 | 517444 | 3662.K03.gz43_519797 | M00086087C:D04 | chiron(cc187- |
| | | | | ProstateCancer4 + 4) |
| 1135 | 1131409 | 3662.L05.gz43_519830 | M00086090B:B09 | chiron(cc187- |
| | | | | ProstateCancer4 + 4) |
| 1136 | 1082403 | 3662.N24.gz43_520136 | M00086097C:B10 | chiron(cc187- |
| | | | | ProstateCancer4 + 4) |
| 1137 | 1112545 | 3662.O02.gz43_519785 | M00086097D:D12 | chiron(cc187- |
| | | | | ProstateCancer4 + 4) |
| 1138 | 1240756 | 3662.P03.gz43_519802 | M00086103D:E08 | chiron(cc187- |
| | | | | ProstateCancer4 + 4) |
| 1139 | 1257531 | 3663.A09.gz43_520267 | M00086106D:H01 | chiron(cc187- |
| | | | | ProstateCancer4 + 4) |
| 1140 | 1052394 | 3663.C08.gz43_520253 | M00086112C:A01 | chiron(cc187- |
| | | | | ProstateCancer4 + 4) |
| 1141 | 1250373 | 3663.C19.gz43_520429 | M00086114D:G11 | chiron(cc187- |
| | | | | ProstateCancer4 + 4) |
| 1142 | 1245188 | 3663.E04.gz43_520191 | M00086120A:D11 | chiron(cc187- |
| | | | | ProstateCancer4 + 4) |
| 1143 | 845931 | 3663.F15.gz43_520368 | M00086126C:D09 | chiron(cc187- |
| | | | | ProstateCancer4 + 4) |
| 1144 | 742875 | 3663.F22.gz43_520480 | M00086127C:C05 | chiron(cc187- |
| | | | | ProstateCancer4 + 4) |
| 1145 | 540730 | 3663.G01.gz43_520145 | M00086128A:D09 | chiron(cc187- |
| | | | | ProstateCancer4 + 4) |
| 1146 | 1261035 | 3663.G08.gz43_520257 | M00086128D:H10 | chiron(cc187- |
| | | | | ProstateCancer4 + 4) |
| 1147 | 452330 | 3663.H20.gz43_520450 | M00086136C:B06 | chiron(cc187- |
| | | | | ProstateCancer4 + 4) |
| 1148 | 552142 | 3663.J06.gz43_520228 | M00086143B:B08 | chiron(cc187- |
| | | | | ProstateCancer4 + 4) |
| 1149 | 1137259 | 3663.J16.gz43_520388 | M00086145A:F07 | chiron(cc187- |
| | | | | ProstateCancer4 + 4) |
| 1150 | 1227497 | 3663.K02.gz43_520165 | M00086146D:A09 | chiron(cc187- |
| | | | | ProstateCancer4 + 4) |
| 1151 | 439297 | 3663.K13.gz43_520341 | M00086149A:F06 | chiron(cc187- |
| | | | | ProstateCancer4 + 4) |
| 1152 | 760580 | 3663.L18.gz43_520422 | M00086155A:G12 | chiron(cc187- |
| | | | | ProstateCancer4 + 4) |
| 1153 | 1224492 | 3663.L24.gz43_520518 | M00086155D:E12 | chiron(cc187- |
| | | | | ProstateCancer4 + 4) |
| 1154 | 1292932 | 3663.M24.gz43_520519 | M00086157D:D03 | chiron(cc187- |
| | | | | ProstateCancer4 + 4) |
| 1155 | 1053514 | 3663.N09.gz43_520280 | M00086159A:F03 | chiron(cc187- |
| | | | | ProstateCancer4 + 4) |
| 1156 | 1055256 | 3663.N10.gz43_520296 | M00086159A:F05 | chiron(cc187- |
| | | | | ProstateCancer4 + 4) |
| 1157 | 189355 | 3663.N12.gz43_520328 | M00086159B:E04 | chiron(cc187- |
| | | | | ProstateCancer4 + 4) |
| 1158 | 644174 | 3663.N16.gz43_520392 | M00086159D:D01 | chiron(cc187- |
| | | | | ProstateCancer4 + 4) |
| 1159 | 727764 | 3663.O07.gz43_520249 | M00086160D:F08 | chiron(cc187- |
| | | | | ProstateCancer4 + 4) |
| 1160 | 1255455 | 3663.O09.gz43_520281 | M00086161B:H08 | chiron(cc187- |
| | | | | ProstateCancer4 + 4) |
| 1161 | 1292996 | 3664.A11.gz43_520683 | M00086166D:H04 | chiron(cc187- |
| | | | | ProstateCancer4 + 4) |
| 1162 | 727730 | 3664.C21.gz43_520845 | M00086175C:D06 | chiron(cc187- |
| | | | | ProstateCancer4 + 4) |
| 1163 | 189355 | 3664.D06.gz43_520606 | M00086176C:A06 | chiron(cc187- |
| | | | | ProstateCancer4 + 4) |
| 1164 | 965947 | 3664.D12.gz43_520702 | M00086178A:D07 | chiron(cc187- |
| | | | | ProstateCancer4 + 4) |
| 1165 | 1261035 | 3664.D17.gz43_520782 | M00086178D:H12 | chiron(cc187- |
| | | | | ProstateCancer4 + 4) |
| 1166 | 761460 | 3664.E18.gz43_520799 | M00086183A:H04 | chiron(cc187- |
| | | | | ProstateCancer4 + 4) |
| 1167 | 504904 | 3664.E23.gz43_520879 | M00086184C:C10 | chiron(cc187- |
| | | | | ProstateCancer4 + 4) |
| 1168 | 1227968 | 3664.E24.gz43_520895 | M00086184C:D04 | chiron(cc187- |
| | | | | ProstateCancer4 + 4) |
| 1169 | 606040 | 3664.G12.gz43_520705 | M00086191A:G09 | chiron(cc187- |
| | | | | ProstateCancer4 + 4) |
| 1170 | 1292932 | 3664.G20.gz43_520833 | M00086193A:F04 | chiron(cc187- |
| | | | | ProstateCancer4 + 4) |
| 1171 | 1069578 | 3664.H15.gz43_520754 | M00086196A:F07 | chiron(cc187- |
| | | | | ProstateCancer4 + 4) |
| 1172 | 1292439 | 3664.H22.gz43_520866 | M00086197B:A03 | chiron(cc187- |
| | | | | ProstateCancer4 + 4) |
| 1173 | 652651 | 3664.J12.gz43_520708 | M00086202C:A07 | chiron(cc187- |
| | | | | ProstateCancer4 + 4) |
| 1174 | 449276 | 3664.J23.gz43_520884 | M00086203C:H04 | chiron(cc187- |
| | | | | ProstateCancer4 + 4) |
| 1175 | 548217 | 3664.K16.gz43_520773 | M00086206A:E10 | chiron(cc187- |
| | | | | ProstateCancer4 + 4) |
| 1176 | 1055326 | 3664.K19.gz43_520821 | M00086206C:C01 | chiron(cc187- |
| | | | | ProstateCancer4 + 4) |
| 1177 | 1293946 | 3664.L21.gz43_520854 | M00086209B:H12 | chiron(cc187- |
| | | | | ProstateCancer4 + 4) |
| 1178 | 1256564 | 3664.O22.gz43_520873 | M00086225B:E01 | chiron(cc187- |
| | | | | ProstateCancer4 + 4) |
| 1179 | 963880 | 3664.P12.gz43_520714 | M00086227B:E06 | chiron(cc187- |
| | | | | ProstateCancer4 + 4) |
| 1180 | 8045 | 3664.P18.gz43_520810 | M00086228A:F11 | chiron(cc187- |
| | | | | ProstateCancer4 + 4) |
| 1181 | 609914 | 3665.A23.gz43_521259 | M00086233C:F01 | chiron(cc187- |
| | | | | ProstateCancer4 + 4) |
| 1182 | 853696 | 3665.B01.gz43_520908 | M00086233D:A03 | chiron(cc187- |
| | | | | ProstateCancer4 + 4) |
| 1183 | 418439 | 3665.B12.gz43_521084 | M00086235A:F05 | chiron(cc187- |
| | | | | ProstateCancer4 + 4) |
| 1184 | 840852 | 3665.E11.gz43_521071 | M00086247A:G11 | chiron(cc187- |
| | | | | ProstateCancer4 + 4) |
| 1185 | 555115 | 3665.E20.gz43_521215 | M00086248A:H09 | chiron(cc187- |
| | | | | ProstateCancer4 + 4) |
| 1186 | 1061206 | 3665.H20.gz43_521218 | M00086259A:F11 | chiron(cc187- |
| | | | | ProstateCancer4 + 4) |
| 1187 | 733840 | 3665.K01.gz43_520917 | M00086266B:E10 | chiron(cc187- |
| | | | | ProstateCancer4 + 4) |
| 1188 | 732183 | 3665.M01.gz43_520919 | M00086270C:D08 | chiron(cc187- |
| | | | | ProstateCancer4 + 4) |
| 1189 | 1292281 | 3665.M21.gz43_521239 | M00086272D:E04 | chiron(cc187- |
| | | | | ProstateCancer4 + 4) |
| 1190 | 1227352 | 3665.M23.gz43_521271 | M00086272D:H11 | chiron(cc187- |
| | | | | ProstateCancer4 + 4) |
| 1191 | 958844 | 3665.N24.gz43_521288 | M00086276D:F10 | chiron(cc187- |
| | | | | ProstateCancer4 + 4) |
| 1192 | 46 | 3665.O06.gz43_521001 | M00086277B:E06 | chiron(cc187- |
| | | | | ProstateCancer4 + 4) |
| 1193 | 1138627 | 3665.O14.gz43_521129 | M00086279A:B07 | chiron(cc187- |
| | | | | ProstateCancer4 + 4) |
| 1194 | 1224389 | 3665.O15.gz43_521145 | M00086279C:A08 | chiron(cc187- |
| | | | | ProstateCancer4 + 4) |
| 1195 | 672192 | 3665.O19.gz43_521209 | M00086279D:C07 | chiron(cc187- |
| | | | | ProstateCancer4 + 4) |
| 1196 | 724897 | 3665.O21.gz43_521241 | M00086280A:E02 | chiron(cc187- |
| | | | | ProstateCancer4 + 4) |
| 1197 | 1292389 | 3665.O23.gz43_521273 | M00086280B:G09 | chiron(cc187- |
| | | | | ProstateCancer4 + 4) |
| 1198 | 1274736 | 3665.P13.gz43_521114 | M00086282A:B10 | chiron(cc187- |
| | | | | ProstateCancer4 + 4) |
| 1199 | 1292582 | 3666.A07.gz43_521387 | M00086285B:C10 | chiron(cc187- |
| | | | | ProstateCancer4 + 4) |
| 1200 | 99774 | 3666.A19.gz43_521579 | M00086286B:F08 | chiron(cc187- |
| | | | | ProstateCancer4 + 4) |
| 1201 | 742101 | 3666.A24.gz43_521659 | M00086286C:H02 | chiron(cc187- |
| | | | | ProstateCancer4 + 4) |
| 1202 | 766134 | 3666.B11.gz43_521452 | M00086288A:B05 | chiron(cc187- |
| | | | | ProstateCancer4 + 4) |
| 1203 | 1250373 | 3666.C18.gz43_521565 | M00086291A:E10 | chiron(cc187- |
| | | | | ProstateCancer4 + 4) |
| 1204 | 1088847 | 3666.D02.gz43_521310 | M00086291D:B08 | chiron(cc187- |
| | | | | ProstateCancer4 + 4) |
| 1205 | 1293946 | 3666.D11.gz43_521454 | M00086294B:E11 | chiron(cc187- |
| | | | | ProstateCancer4 + 4) |
| 1206 | 3462 | 3666.D15.gz43_521518 | M00086294C:G05 | chiron(cc187- |
| | | | | ProstateCancer4 + 4) |
| 1207 | 727764 | 3666.D16.gz43_521534 | M00086294D:F08 | chiron(cc187- |
| | | | | ProstateCancer4 + 4) |
| 1208 | 396724 | 3666.F22.gz43_521632 | M00086301A:D04 | chiron(cc187- |
| | | | | ProstateCancer4 + 4) |
| 1209 | 1142019 | 3666.G12.gz43_521473 | M00086302A:E06 | chiron(cc187- |
| | | | | ProstateCancer4 + 4) |
| 1210 | 765500 | 3666.I12.gz43_521475 | M00086310A:F04 | chiron(cc187- |
| | | | | ProstateCancer4 + 4) |
| 1211 | 1197259 | 3666.L01.gz43_521302 | M00086322A:E02 | chiron(cc187- |
| | | | | ProstateCancer4 + 4) |
| 1212 | 1138472 | 3666.L06.gz43_521382 | M00086322D:D05 | chiron(cc187- |
| | | | | ProstateCancer4 + 4) |
| 1213 | 869453 | 3666.L11.gz43_521462 | M00086323B:C04 | chiron(cc187- |
| | | | | ProstateCancer4 + 4) |
| 1214 | 1088251 | 3666.L23.gz43_521654 | M00086324D:D01 | chiron(cc187- |
| | | | | ProstateCancer4 + 4) |
| 1215 | 1292413 | 3666.M16.gz43_521543 | M00086327B:D10 | chiron(cc187- |
| | | | | ProstateCancer4 + 4) |
| 1216 | 1274736 | 3666.N06.gz43_521384 | M00086328B:G12 | chiron(cc187- |
| | | | | ProstateCancer4 + 4) |
| 1217 | 1259955 | 3667.A15.gz43_524557 | M00086336B:A08 | chiron(cc187- |
| | | | | ProstateCancer4 + 4) |
| 1218 | 8045 | 3754.A08.gz43_532949 | M00083799D:F10 | chiron(cc187-NormBPHProstate) |
| 1219 | 1227999 | 3754.A13.gz43_533029 | M00083800C:E07 | chiron(cc187-NormBPHProstate) |
| 1220 | 637915 | 3754.A16.gz43_533077 | M00083801B:H03 | chiron(cc187-NormBPHProstate) |
| 1221 | 764978 | 3754.B04.gz43_532886 | M00083803B:F11 | chiron(cc187-NormBPHProstate) |
| 1222 | 500919 | 3754.805.gz43_532902 | M00083803B:F12 | chiron(cc187-NormBPHProstate) |
| 1223 | 1224133 | 3754.B07.gz43_532934 | M00083803C:F03 | chiron(cc187-NormBPHProstate) |
| 1224 | 831638 | 3754.B08.gz43_532950 | M00083804A:H12 | chiron(cc187-NormBPHProstate) |
| 1225 | 1229792 | 3754.B10.gz43_532982 | M00083804B:C03 | chiron(cc187-NormBPHProstate) |
| 1226 | 1223618 | 3754.C22.gz43_533175 | M00083809B:E08 | chiron(cc187-NormBPHProstate) |
| 1227 | 453767 | 3754.D19.gz43_533128 | M00083812C:G02 | chiron(cc187-NormBPHProstate) |
| 1228 | 733538 | 3754.E12.gz43_533017 | M00083814D:A10 | chiron(cc187-NormBPHProstate) |
| 1229 | 401424 | 3754.E20.gz43_533145 | M00083815C:H08 | chiron(cc187-NormBPHProstate) |
| 1230 | 1081049 | 3754.F01.gz43_532842 | M00083816B:D08 | chiron(cc187-NormBPHProstate) |
| 1231 | 1193124 | 3754.F08.gz43_532954 | M00083817B:A11 | chiron(cc187-NormBPHProstate) |
| 1232 | 1085638 | 3754.F11.gz43_533002 | M00083817B:G09 | chiron(cc187-NormBPHProstate) |
| 1233 | 427093 | 3754.F15.gz43_533066 | M00083817D:A08 | chiron(cc187-NormBPHProstate) |
| 1234 | 514090 | 3754.F20.gz43_533146 | M00083818A:E09 | chiron(cc187-NormBPHProstate) |
| 1235 | 1080401 | 3754.G03.gz43_532875 | M00083818C:A02 | chiron(cc187-NormBPHProstate) |
| 1236 | 567700 | 3754.G08.gz43_532955 | M00083819B:E10 | chiron(cc187-NormBPHProstate) |
| 1237 | 828695 | 3754.G18.gz43_533115 | M00083820B:C03 | chiron(cc187-NormBPHProstate) |
| 1238 | 717583 | 3754.H08.gz43_532956 | M00083831D:H11 | chiron(cc187-NormBPHProstate) |
| 1239 | 454214 | 3754.I01.gz43_532845 | M00083834B:F09 | chiron(cc187-NormBPHProstate) |
| 1240 | 169762 | 3754.I03.gz43_532877 | M00083834C:E02 | chiron(cc187-NormBPHProstate) |
| 1241 | 1227912 | 3754.J01.gz43_532846 | M00083838A:E05 | chiron(cc187-NormBPHProstate) |
| 1242 | 1218793 | 3754.J05.gz43_532910 | M00083838C:F07 | chiron(cc187-NormBPHProstate) |
| 1243 | 855095 | 3754.J10.gz43_532990 | M00083839A:H03 | chiron(cc187-NormBPHProstate) |
| 1244 | 1176992 | 3754.J12.gz43_533022 | M00083839B:G09 | chiron(cc187-NormBPHProstate) |
| 1245 | 1073767 | 3754.J24.gz43_533214 | M00083841A:G01 | chiron(cc187-NormBPHProstate) |
| 1246 | 452015 | 3754.K14.gz43_533055 | M00083844A:E12 | chiron(cc187-NormBPHProstate) |
| 1247 | 1224454 | 3754.K17.gz43_533103 | M00083844B:C04 | chiron(cc187-NormBPHProstate) |
| 1248 | 961757 | 3754.K20.gz43_533151 | M00083844C:C04 | chiron(cc187-NormBPHProstate) |
| 1249 | 403738 | 3754.M08.gz43_532961 | M00083849C:F11 | chiron(cc187-NormBPHProstate) |
| 1250 | 455386 | 3754.N16.gz43_533090 | M00084246A:D03 | chiron(cc187-NormBPHProstate) |
| 1251 | 556339 | 3754.N19.gz43_533138 | M00084246B:D10 | chiron(cc187-NormBPHProstate) |
| 1252 | 660907 | 3754.N22.gz43_533186 | M00084246B:H03 | chiron(cc187-NormBPHProstate) |
| 1253 | 404308 | 3754.O18.gz43_533123 | M00084248B:C06 | chiron(cc187-NormBPHProstate) |
| 1254 | 1202570 | 3754.O23.gz43_533203 | M00084248D:H09 | chiron(cc187-NormBPHProstate) |
| 1255 | 1226932 | 3754.P13.gz43_533044 | M00084251D:C05 | chiron(cc187-NormBPHProstate) |
| 1256 | 1054807 | 3754.P17.gz43_533108 | M00084252B:H01 | chiron(cc187-NormBPHProstate) |
| 1257 | 1053061 | 3756.A02.gz43_533237 | M00085806B:A10 | chiron(cc187- |
| | | | | ProstateCancer4 + 4) |
| 1258 | 1079765 | 3756.A11.gz43_533381 | M00085825C:D12 | chiron(cc187- |
| | | | | ProstateCancer4 + 4) |
| 1259 | 1092391 | 3756.A13.gz43_533413 | M00085830B:E09 | chiron(cc187- |
| | | | | ProstateCancer4 + 4) |
| 1260 | 1226388 | 3756.B03.gz43_533254 | M00085809A:E04 | chiron(cc187- |
| | | | | ProstateCancer4 + 4) |
| 1261 | 256 | 3756.B04.gz43_533270 | M00085811B:D12 | chiron(cc187- |
| | | | | ProstateCancer4 + 4) |
| 1262 | 730228 | 3756.B15.gz43_533446 | M00085835D:F06 | chiron(cc187- |
| | | | | ProstateCancer4 + 4) |
| 1263 | 1255037 | 3756.B21.gz43_533542 | M00085859B:A11 | chiron(cc187- |
| | | | | ProstateCancer4 + 4) |
| 1264 | 1080716 | 3756.B22.gz43_533558 | M00085861C:A03 | chiron(cc187- |
| | | | | ProstateCancer4 + 4) |
| 1265 | 777485 | 3756.C06.gz43_533303 | M00085814B:G08 | chiron(cc187- |
| | | | | ProstateCancer4 + 4) |
| 1266 | 449687 | 3756.C16.gz43_533463 | M00085837C:H08 | chiron(cc187- |
| | | | | ProstateCancer4 + 4) |
| 1267 | 1259906 | 3756.D08.gz43_533336 | M00085819D:C02 | chiron(cc187- |
| | | | | ProstateCancer4 + 4) |
| 1268 | 2454 | 3756.D18.gz43_533496 | M00085846A:H10 | chiron(cc187- |
| | | | | ProstateCancer4 + 4) |
| 1269 | 1106514 | 3756.D24.gz43_533592 | M00085804B:F09 | chiron(cc187- |
| | | | | ProstateCancer4 + 4) |
| 1270 | 1053078 | 3756.E01.gz43_533225 | M00085805A:G07 | chiron(cc187- |
| | | | | ProstateCancer4 + 4) |
| 1271 | 1053014 | 3756.E06.gz43_533305 | M00085814C:C12 | chiron(cc187- |
| | | | | ProstateCancer4 + 4) |
| 1272 | 558976 | 3756.E12.gz43_533401 | M00085827C:F03 | chiron(cc187- |
| | | | | ProstateCancer4 + 4) |
| 1273 | 492111 | 3756.E22.gz43_533561 | M00085860D:H02 | chiron(cc187- |
| | | | | ProstateCancer4 + 4) |
| 1274 | 9249 | 3756.F11.gz43_533386 | M00085826D:B03 | chiron(cc187- |
| | | | | ProstateCancer4 + 4) |
| 1275 | 1138799 | 3756.F16.gz43_533466 | M00085839B:B12 | chiron(cc187- |
| | | | | ProstateCancer4 + 4) |
| 1276 | 1052216 | 3756.G07.gz43_533323 | M00085817B:B08 | chiron(cc187- |
| | | | | ProstateCancer4 + 4) |
| 1277 | 714840 | 3756.G12.gz43_533403 | M00085827D:D01 | chiron(cc187- |
| | | | | ProstateCancer4 + 4) |
| 1278 | 372541 | 3756.G14.gz43_533435 | M00085832D:G02 | chiron(cc187- |
| | | | | ProstateCancer4 + 4) |
| 1279 | 374098 | 3756.I03.gz43_533261 | M00085808C:E12 | chiron(cc187- |
| | | | | ProstateCancer4 + 4) |
| 1280 | 8756 | 3756.J05.gz43_533294 | M00085814A:C02 | chiron(cc187- |
| | | | | ProstateCancer4 + 4) |
| 1281 | 486961 | 3756.K03.gz43_533263 | M00085808D:E01 | chiron(cc187- |
| | | | | ProstateCancer4 + 4) |
| 1282 | 617714 | 3756.K07.gz43_533327 | M00085817C:C10 | chiron(cc187- |
| | | | | ProstateCancer4 + 4) |
| 1283 | 2946 | 3756.K15.gz43_533455 | M00085835B:E11 | chiron(cc187- |
| | | | | ProstateCancer4 + 4) |
| 1284 | 1227963 | 3756.K18.gz43_533503 | M00085844C:H11 | chiron(cc187- |
| | | | | ProstateCancer4 + 4) |
| 1285 | 1206161 | 3756.K20.gz43_533535 | M00085854C:E06 | chiron(cc187- |
| | | | | ProstateCancer4 + 4) |
| 1286 | 608560 | 3756.L02.gz43_533248 | M00085807D:G11 | chiron(cc187- |
| | | | | ProstateCancer4 + 4) |
| 1287 | 16155 | 3756.L03.gz43_533264 | M00085810A:A10 | chiron(cc187- |
| | | | | ProstateCancer4 + 4) |
| 1288 | 1194431 | 3756.L19.gz43_533520 | M00085854A:D09 | chiron(cc187- |
| | | | | ProstateCancer4 + 4) |
| 1289 | 174730 | 3756.M06.gz43_533313 | M00085815C:E11 | chiron(cc187- |
| | | | | ProstateCancer4 + 4) |
| 1290 | 372741 | 3756.M07.gz43_533329 | M00085817C:G05 | chiron(cc187- |
| | | | | ProstateCancer4 + 4) |
| 1291 | 380070 | 3756.M20.gz43_533537 | M00085854C:F04 | chiron(cc187- |
| | | | | ProstateCancer4 + 4) |
| 1292 | 974635 | 3756.N18.gz43_533506 | M00085849D:G06 | chiron(cc187- |
| | | | | ProstateCancer4 + 4) |
| 1293 | 1252747 | 3756.N21.gz43_533554 | M00085860B:D10 | chiron(cc187- |
| | | | | ProstateCancer4 + 4) |
| 1294 | 525759 | 3756.O03.gz43_533267 | M00085809A:C05 | chiron(cc187- |
| | | | | ProstateCancer4 + 4) |
| 1295 | 386109 | 3756.O07.gz43_533331 | M00085817D:C04 | chiron(cc187- |
| | | | | ProstateCancer4 + 4) |
| 1296 | 401342 | 3756.O08.gz43_533347 | M00085819C:F06 | chiron(cc187- |
| | | | | ProstateCancer4 + 4) |
| 1297 | 1260177 | 3756.P08.gz43_533348 | M00085820D:D02 | chiron(cc187- |
| | | | | ProstateCancer4 + 4) |
| 1298 | 720454 | 3759.C01.gz43_533607 | M00085068C:B03 | chiron(cc187-NormBPHProstate) |
| 1299 | 875843 | 3759.D15.gz43_533832 | M00085092D:D09 | chiron(cc187-NormBPHProstate) |
| 1300 | 402851 | 3759.H08.gz43_533724 | M00085082C:B04 | chiron(cc187-NormBPHProstate) |
| 1301 | 21370 | 3759.H15.gz43_533836 | M00085100A:A12 | chiron(cc187-NormBPHProstate) |
| 1302 | 1196268 | 3759.H17.gz43_533868 | M00085105C:D01 | chiron(cc187-NormBPHProstate) |
| 1303 | 185432 | 3759.H23.gz43_533964 | M00085066B:D12 | chiron(cc187-NormBPHProstate) |
| 1304 | 1268161 | 3759.I05.gz43_533677 | M00085076C:A07 | chiron(cc187-NormBPHProstate) |
| 1305 | 44737 | 3759.I19.gz43_533901 | M00085107D:H08 | chiron(cc187-NormBPHProstate) |
| 1306 | 400734 | 3759.K05.gz43_533679 | M00085076C:H01 | chiron(cc187-NormBPHProstate) |
| 1307 | 413573 | 3759.K17.gz43_533871 | M00085104C:A10 | chiron(cc187-NormBPHProstate) |
| 1308 | 1066654 | 3759.L02.gz43_533632 | M00085071B:D07 | chiron(cc187-NormBPHProstate) |
| 1309 | 1226365 | 3759.L09.gz43_533744 | M00085084A:E12 | chiron(cc187-NormBPHProstate) |
| 1310 | 15759 | 3759.L10.gz43_533760 | M00085085C:D10 | chiron(cc187-NormBPHProstate) |
| 1311 | 140372 | 3759.L15.gz43_533840 | M00085100A:H07 | chiron(cc187-NormBPHProstate) |
| 1312 | 1138572 | 3759.L24.gz43_533984 | M00085068B:A07 | chiron(cc187-NormBPHProstate) |
| 1313 | 6790 | 3759.M19.gz43_533905 | M00085108A:C12 | chiron(cc187-NormBPHProstate) |
| 1314 | 1228147 | 3759.N08.gz43_533730 | M00085083A:E04 | chiron(cc187-NormBPHProstate) |
| 1315 | 43678 | 3759.N16.gz43_533858 | M00085103D:H12 | chiron(cc187-NormBPHProstate) |
| 1316 | 1116992 | 3759.N23.gz43_533970 | M00085066D:A05 | chiron(cc187-NormBPHProstate) |
| 1317 | 393261 | 3759.O16.gz43_533859 | M00085101C:H03 | chiron(cc187-NormBPHProstate) |
| 1318 | 378459 | 3759.P03.gz43_533652 | M00085074B:A07 | chiron(cc187-NormBPHProstate) |
| 1319 | 242707 | 3759.P13.gz43_533812 | M00085090C:C09 | chiron(cc187-NormBPHProstate) |
| 1320 | 69 | 3759.P15.gz43_533844 | M00085100B:C12 | chiron(cc187-NormBPHProstate) |
| 1321 | 12613 | 3759.P17.gz43_533876 | M00085105D:H02 | chiron(cc187-NormBPHProstate) |
| 1322 | 1052108 | 3762.A09.gz43_534117 | M00084772C:G12 | chiron(cc187-NormBPHProstate) |
| 1323 | 1191547 | 3762.A16.gz43_534229 | M00084773C:H08 | chiron(cc187-NormBPHProstate) |
| 1324 | 204985 | 3762.A19.gz43_534277 | M00084773C:F08 | chiron(cc187-NormBPHProstate) |
| 1325 | 1054813 | 3762.A20.gz43_534293 | M00084773C:D04 | chiron(cc187-NormBPHProstate) |
| 1326 | 687223 | 3762.B05.gz43_534054 | M00084774B:C10 | chiron(cc187-NormBPHProstate) |
| 1327 | 1225719 | 3762.B15.gz43_534214 | M00084775C:E05 | chiron(cc187-NormBPHProstate) |
| 1328 | 1139522 | 3762.C20.gz43_534295 | M00084779D:H10 | chiron(cc187-NormBPHProstate) |
| 1329 | 408711 | 3762.C23.gz43_534343 | M00084779B:D03 | chiron(cc187-NormBPHProstate) |
| 1330 | 770834 | 3762.D03.gz43_534024 | M00084780C:F08 | chiron(cc187-NormBPHProstate) |
| 1331 | 961790 | 3762.D04.gz43_534040 | M00084780D:D07 | chiron(cc187-NormBPHProstate) |
| 1332 | 1118470 | 3762.D18.gz43_534264 | M00084781A:H09 | chiron(cc187-NormBPHProstate) |
| 1333 | 140314 | 3762.D19.gz43_534280 | M00084781A:E05 | chiron(cc187-NormBPHProstate) |
| 1334 | 684638 | 3762.D22.gz43_534328 | M00084781A:A05 | chiron(cc187-NormBPHProstate) |
| 1335 | 1054648 | 3762.E01.gz43_533993 | M00084782D:H08 | chiron(cc187-NormBPHProstate) |
| 1336 | 454214 | 3762.E10.gz43_534137 | M00084782D:D10 | chiron(cc187-NormBPHProstate) |
| 1337 | 77856 | 3762.E15.gz43_534217 | M00084783C:A09 | chiron(cc187-NormBPHProstate) |
| 1338 | 4745 | 3762.E23.gz43_534345 | M00084783C:G08 | chiron(cc187-NormBPHProstate) |
| 1339 | 557063 | 3762.F08.gz43_534106 | M00084784B:A02 | chiron(cc187-NormBPHProstate) |
| 1340 | 1087318 | 3762.F22.gz43_534330 | M00084787B:D12 | chiron(cc187-NormBPHProstate) |
| 1341 | 1056369 | 3762.G18.gz43_534267 | M00084789D:B10 | chiron(cc187-NormBPHProstate) |
| 1342 | 996888 | 3762.H12.gz43_534172 | M00084791D:D01 | chiron(cc187-NormBPHProstate) |
| 1343 | 968647 | 3762.I07.gz43_534093 | M00084794B:C01 | chiron(cc187-NormBPHProstate) |
| 1344 | 453767 | 3762.J03.gz43_534030 | M00084796D:B01 | chiron(cc187-NormBPHProstate) |
| 1345 | 1014734 | 3762.J18.gz43_534270 | M00084799C:E08 | chiron(cc187-NormBPHProstate) |
| 1346 | 86612 | 3762.K02.gz43_534015 | M00084800B:H09 | chiron(cc187-NormBPHProstate) |
| 1347 | 551691 | 3762.K20.gz43_534303 | M00084802A:H09 | chiron(cc187-NormBPHProstate) |
| 1348 | 691512 | 3762.L18.gz43_534272 | M00084804C:H10 | chiron(cc187-NormBPHProstate) |
| 1349 | 1259069 | 3762.L20.gz43_534304 | M00084804B:E01 | chiron(cc187-NormBPHProstate) |
| 1350 | 683650 | 3762.M04.gz43_534049 | M00084805D:E02 | chiron(cc187-NormBPHProstate) |
| 1351 | 447151 | 3762.M17.gz43_534257 | M00084807D:F07 | chiron(cc187-NormBPHProstate) |
| 1352 | 1052058 | 3762.M23.gz43_534353 | M00084808D:A07 | chiron(cc187-NormBPHProstate) |

Summary of Polynucleotides of the Invention

Table 11 (inserted prior to claims) provides a summary of polynucleotides isolated as described. Specifically, Table 11 provides: 1) the SEQ ID NO (“SEQ ID”) assigned to each sequence for use in the present specification; 2) the Cluster Identification No. (“CLUSTER”); 3) the Sequence Name assigned to each sequence; 3) the sequence name (“SEQ NAME”) used as an internal identifier of the sequence; 4) the name assigned to the clone from which the sequence was isolated (“CLONE ID”); and 5) the name of the library from which the sequence was isolated (“LIBRARY”). Because at least some of the provided polynucleotides represent partial mRNA transcripts, two or more polynucleotides may represent different regions of the same mRNA transcript and the same gene and/or may be contained within the same clone. Thus, for example, if two or more SEQ ID NOS: are identified as belonging to the same clone, then either sequence can be used to obtain the full-length mRNA or gene. Clones which comprise the sequences described herein were deposited as set out in the tables indicated below (see Example entitled “Deposit Information”).

### Example 18

### Contig Assembly

The sequences of the polynucleotides provided in the present invention can be used to extend the sequence information of the gene to which the polynucleotides correspond (e.g., a gene, or mRNA encoded by the gene, having a sequence of the polynucleotide described herein). This expanded sequence information can in turn be used to further characterize the corresponding gene, which in turn provides additional information about the nature of the gene product (e.g., the normal function of the gene product). The additional information can serve to provide additional evidence of the gene product's use as a therapeutic target, and provide further guidance as to the types of agents that can modulate its activity.

For example, a contig was assembled using the sequence of a polynucleotide described herein. A “contig” is a contiguous sequence of nucleotides that is assembled from nucleic acid sequences having overlapping (e.g., shared or substantially similar) sequence information. The sequences of publicly-available ESTs (Expressed Sequence Tags) and the sequences of various of the above-described polynucleotides were used in the contig assembly. The contig was assembled using the software program Sequencher, version 4.05, according to the manufacturer's instructions. The sequence information obtained in the contig assembly was then used to obtain a consensus sequence derived from the contig using the Sequencher program. The resulting consensus sequence was used to search both the public databases as well as databases internal to the applicants to match the consensus polynucleotide with homology data and/or differential gene expressed data.

The final result provided the sequences listed as SEQ ID NOS: 1353-1561 in the accompanying Sequence Listing and summarized in Tables 12 and 13 (inserted prior to claims). Table 12 provides a summary of the consensus sequences assembled as described. Specifically, Table 3 provides: 1) the SEQ ID NO (“SEQ ID”) assigned to each consensus sequence for use in the present specification; 2) the Cluster Identification No. (“CLUSTER”); and 3) the consensus sequence name (“CONSENSUS SEQ NAME”) used as an internal identifier of the sequence.

TABLE 12

| SEQ ID | CLUSTER | CONSENSUS SEQ NAME |
|---|---|---|
| 1353 | 46 | Clu46.con_5 |
| 1354 | 8293 | Clu8293.con_1 |
| 1355 | 34201 | Clu34201.con_1 |
| 1356 | 48343 | Clu48343.con_1 |
| 1357 | 89833 | Clu89833.con_2 |
| 1358 | 140314 | Clu140314.con_2 |
| 1359 | 141870 | Clu141870.con_1 |
| 1360 | 180092 | Clu180092.con_1 |
| 1361 | 189355 | Clu189355.con_1 |
| 1362 | 234667 | Clu234667.con_1 |
| 1363 | 242901 | Clu242901.con_1 |
| 1364 | 307985 | Clu307985.con_1 |
| 1365 | 387610 | Clu387610.con_1 |
| 1366 | 389995 | Clu389995.con_2 |
| 1367 | 393948 | Clu393948.con_1 |
| 1368 | 397313 | Clu397313.con_1 |
| 1369 | 398439 | Clu398439.con_1 |
| 1370 | 402150 | Clu402150.con_1 |
| 1371 | 402789 | Clu402789.con_1 |
| 1372 | 403488 | Clu403488.con_1 |
| 1373 | 413505 | Clu413505.con_1 |
| 1374 | 418320 | Clu418320.con_1 |
| 1375 | 424678 | Clu424678.con_1 |
| 1376 | 426138 | Clu426138.con_1 |
| 1377 | 427904 | Clu427904.con_1 |
| 1378 | 447151 | Clu447151.con_1 |
| 1379 | 452564 | Clu452564.con_1 |
| 1380 | 454826 | Clu454826.con_1 |
| 1381 | 460499 | Clu460499.con_1 |
| 1382 | 477110 | Clu477110.con_1 |
| 1383 | 477708 | Clu477708.con_1 |
| 1384 | 478212 | Clu478212.con_1 |
| 1385 | 484459 | Clu484459.con_1 |
| 1386 | 494499 | Clu494499.con_1 |
| 1387 | 494890 | Clu494890.con_1 |
| 1388 | 500919 | Clu500919.con_1 |
| 1389 | 504038 | Clu504038.con_1 |
| 1390 | 504904 | Clu504904.con_2 |
| 1391 | 505750 | Clu505750.con_1 |
| 1392 | 517444 | Clu517444.con_1 |
| 1393 | 528281 | Clu528281.con_1 |
| 1394 | 529709 | Clu529709.con_1 |
| 1395 | 532062 | Clu532062.con_1 |
| 1396 | 542301 | Clu542301.con_1 |
| 1397 | 542825 | Clu542825.con_1 |
| 1398 | 548217 | Clu548217.con_1 |
| 1399 | 548277 | Clu548277.con_1 |
| 1400 | 554189 | Clu554189.con_2 |
| 1401 | 555115 | Clu555115.con_2 |
| 1402 | 555967 | Clu555967.con_1 |
| 1403 | 557717 | Clu557717.con_1 |
| 1404 | 566548 | Clu566548.con_1 |
| 1405 | 567700 | Clu567700.con_2 |
| 1406 | 573169 | Clu573169.con_1 |
| 1407 | 585099 | Clu585099.con_1 |
| 1408 | 585899 | Clu585899.con_1 |
| 1409 | 609914 | Clu609914.con_1 |
| 1410 | 613411 | Clu613411.con_1 |
| 1411 | 621702 | Clu621702.con_1 |
| 1412 | 637915 | Clu637915.con_1 |
| 1413 | 640157 | Clu640157.con_1 |
| 1414 | 640277 | Clu640277.con_1 |
| 1415 | 645986 | Clu645986.con_1 |
| 1416 | 647587 | Clu647587.con_2 |
| 1417 | 652651 | Clu652651.con_1 |
| 1418 | 653817 | Clu653817.con_1 |
| 1419 | 660907 | Clu660907.con_1 |
| 1420 | 661802 | Clu661802.con_1 |
| 1421 | 676665 | Clu676665.con_1 |
| 1422 | 684638 | Clu684638.con_1 |
| 1423 | 691512 | Clu691512.con_1 |
| 1424 | 700354 | Clu700354.con_1 |
| 1425 | 708025 | Clu708025.con_1 |
| 1426 | 710194 | Clu710194.con_1 |
| 1427 | 733840 | Clu733840.con_1 |
| 1428 | 734568 | Clu734568.con_1 |
| 1429 | 742101 | Clu742101.con_1 |
| 1430 | 747805 | Clu747805.con_1 |
| 1431 | 761460 | Clu761460.con_1 |
| 1432 | 765500 | Clu765500.con_1 |
| 1433 | 770834 | Clu770834.con_1 |
| 1434 | 774520 | Clu774520.con_1 |
| 1435 | 777670 | Clu777670.con_1 |
| 1436 | 812031 | Clu812031.con_1 |
| 1437 | 821536 | Clu821536.con_1 |
| 1438 | 823271 | Clu823271.con_1 |
| 1439 | 845354 | Clu845354.con_1 |
| 1440 | 846056 | Clu846056.con_1 |
| 1441 | 854573 | Clu854573.con_1 |
| 1442 | 863768 | Clu863768.con_1 |
| 1443 | 869453 | Clu869453.con_1 |
| 1444 | 875978 | Clu875978.con_2 |
| 1445 | 893981 | Clu893981.con_1 |
| 1446 | 945247 | Clu945247.con_1 |
| 1447 | 947168 | Clu947168.con_1 |
| 1448 | 961757 | Clu961757.con_1 |
| 1449 | 968647 | Clu968647.con_1 |
| 1450 | 970165 | Clu970165.con_1 |
| 1451 | 991366 | Clu991366.con_1 |
| 1452 | 1014734 | Clu1014734.con_1 |
| 1453 | 1037887 | Clu1037887.con_1 |
| 1454 | 1052399 | Clu1052399.con_1 |
| 1455 | 1052466 | Clu1052466.con_1 |
| 1456 | 1053121 | Clu1053121.con_1 |
| 1457 | 1053514 | Clu1053514.con_1 |
| 1458 | 1053747 | Clu1053747.con_1 |
| 1459 | 1053799 | Clu1053799.con_1 |
| 1460 | 1053854 | Clu1053854.con_1 |
| 1461 | 1054038 | Clu1054038.con_1 |
| 1462 | 1054069 | Clu1054069.con_1 |
| 1463 | 1054074 | Clu1054074.con_1 |
| 1464 | 1054807 | Clu1054807.con_1 |
| 1465 | 1054813 | Clu1054813.con_1 |
| 1466 | 1054884 | Clu1054884.con_1 |
| 1467 | 1055018 | Clu1055018.con_1 |
| 1468 | 1055063 | Clu1055063.con_1 |
| 1469 | 1055089 | Clu1055089.con_1 |
| 1470 | 1055256 | Clu1055256.con_1 |
| 1471 | 1055326 | Clu1055326.con_1 |
| 1472 | 1056369 | Clu1056369.con_1 |
| 1473 | 1059332 | Clu1059332.con_1 |
| 1474 | 1059445 | Clu1059445.con_1 |
| 1475 | 1060021 | Clu1060021.con_1 |
| 1476 | 1061206 | Clu1061206.con_1 |
| 1477 | 1062537 | Clu1062537.con_1 |
| 1478 | 1064975 | Clu1064975.con_1 |
| 1479 | 1065531 | Clu1065531.con_1 |
| 1480 | 1066041 | Clu1066041.con_1 |
| 1481 | 1069578 | Clu1069578.con_1 |
| 1482 | 1069632 | Clu1069632.con_1 |
| 1483 | 1073767 | Clu1073767.con_1 |
| 1484 | 1074160 | Clu1074160.con_1 |
| 1485 | 1076930 | Clu1076930.con_1 |
| 1486 | 1077033 | Clu1077033.con_1 |
| 1487 | 1079196 | Clu1079196.con_1 |
| 1488 | 1079863 | Clu1079863.con_1 |
| 1489 | 1085638 | Clu1085638.con_1 |
| 1490 | 1085645 | Clu1085645.con_1 |
| 1491 | 1088847 | Clu1088847.con_1 |
| 1492 | 1088930 | Clu1088930.con_1 |
| 1493 | 1108332 | Clu1108332.con_2 |
| 1494 | 1110143 | Clu1110143.con_1 |
| 1495 | 1116087 | Clu1116087.con_1 |
| 1496 | 1131409 | Clu1131409.con_1 |
| 1497 | 1132413 | Clu1132413.con_1 |
| 1498 | 1136803 | Clu1136803.con_1 |
| 1499 | 1138419 | Clu1138419.con_1 |
| 1500 | 1138593 | Clu1138593.con_1 |
| 1501 | 1139522 | Clu1139522.con_2 |
| 1502 | 1139691 | Clu1139691.con_1 |
| 1503 | 1142019 | Clu1142019.con_1 |
| 1504 | 1171518 | Clu1171518.con_2 |
| 1505 | 1176182 | Clu1176182.con_1 |
| 1506 | 1182447 | Clu1182447.con_1 |
| 1507 | 1183079 | Clu1183079.con_1 |
| 1508 | 1184134 | Clu1184134.con_1 |
| 1509 | 1189027 | Clu1189027.con_1 |
| 1510 | 1191547 | Clu1191547.con_1 |
| 1511 | 1193236 | Clu1193236.con_1 |
| 1512 | 1210953 | Clu1210953.con_1 |
| 1513 | 1211899 | Clu1211899.con_1 |
| 1514 | 1218793 | Clu1218793.con_1 |
| 1515 | 1223271 | Clu1223271.con_1 |
| 1516 | 1223477 | Clu1223477.con_1 |
| 1517 | 1223907 | Clu1223907.con_1 |
| 1518 | 1223938 | Clu1223938.con_1 |
| 1519 | 1223948 | Clu1223948.con_1 |
| 1520 | 1224039 | Clu1224039.con_1 |
| 1521 | 1224226 | Clu1224226.con_1 |
| 1522 | 1224379 | Clu1224379.con_1 |
| 1523 | 1224422 | Clu1224422.con_1 |
| 1524 | 1224547 | Clu1224547.con_1 |
| 1525 | 1224704 | Clu1224704.con_1 |
| 1526 | 1224752 | Clu1224752.con_1 |
| 1527 | 1224881 | Clu1224881.con_2 |
| 1528 | 1225500 | Clu1225500.con_1 |
| 1529 | 1225595 | Clu1225595.con_1 |
| 1530 | 1225719 | Clu1225719.con_2 |
| 1531 | 1225734 | Clu1225734.con_1 |
| 1532 | 1226064 | Clu1226064.con_1 |
| 1533 | 1226304 | Clu1226304.con_1 |
| 1534 | 1226413 | Clu1226413.con_1 |
| 1535 | 1226932 | Clu1226932.con_1 |
| 1536 | 1227623 | Clu1227623.con_1 |
| 1537 | 1227781 | Clu1227781.con_1 |
| 1538 | 1227862 | Clu1227862.con_2 |
| 1539 | 1227912 | Clu1227912.con_1 |
| 1540 | 1227968 | Clu1227968.con_1 |
| 1541 | 1228277 | Clu1228277.con_1 |
| 1542 | 1230257 | Clu1230257.con_1 |
| 1543 | 1245188 | Clu1245188.con_1 |
| 1544 | 1250373 | Clu1250373.con_1 |
| 1545 | 1256564 | Clu1256564.con_2 |
| 1546 | 1259069 | Clu1259069.con_2 |
| 1547 | 1274736 | Clu1274736.con_1 |
| 1548 | 1283437 | Clu1283437.con_2 |
| 1549 | 1292262 | Clu1292262.con_1 |
| 1550 | 1292281 | Clu1292281.con_1 |
| 1551 | 1292289 | Clu1292289.con_1 |
| 1552 | 1292413 | Clu1292413.con_1 |
| 1553 | 1292423 | Clu1292423.con_1 |
| 1554 | 1292436 | Clu1292436.con_1 |
| 1555 | 1292439 | Clu1292439.con_1 |
| 1556 | 1292582 | Clu1292582.con_1 |
| 1557 | 1292600 | Clu1292600.con_1 |
| 1558 | 1292932 | Clu1292932.con_1 |
| 1559 | 1292996 | Clu1292996.con_1 |
| 1560 | 1293946 | Clu1293946.con_1 |
| 1561 | 1293972 | Clu1293972.con_1 |

A correlation between the polynucleotide used in consensus sequence assembly as described above and the corresponding consensus sequence is contained in Table 13. Specifically Table 13 provides: 1) the SEQ ID NO of the consensus sequence (“CONSENSUS SEQ ID”); 2) the consensus sequence name (“CONSENSUS SEQ NAME”) used as an internal identifier of the sequence; 3) the SEQ ID NO of the polynucleotide (“POLYNTD SEQ ID”) of SEQ ID NOS: 134-1352 used in assembly of the consensus sequence; and 4) the sequence name (“POLYNTD SEQ NAME”) of the polynucleotide of SEQ ID NOS: 134-1352 used in assembly of the consensus sequence.

TABLE 13

| CON- | | | |
|---|---|---|---|
| SEN- | | | |
| SUS | CONSENSUS SEQ | POLYNTD | |
| SEQ ID | NAME | SEQ ID | POLYNTD SEQ NAME |
| 1353 | Clu46.con_5 | 1192 | 3665.O06.gz43_521001 |
| 1354 | Clu8293.con_1 | 234 | 3547.I16.GZ43_505943 |
| 1355 | Clu34201.con_1 | 491 | 3565.E16.GZ43_508243 |
| 1356 | Clu48343.con_1 | 295 | 3550.O15.GZ43_506317 |
| 1357 | Clu89833.con_2 | 591 | 3574.C14.GZ43_509379 |
| 1357 | Clu89833.con_2 | 840 | 3599.K04.GZ43_512924 |
| 1358 | Clu140314.con_2 | 563 | 3571.H01.GZ43_508792 |
| 1358 | Clu140314.con_2 | 1333 | 3762.D19.gz43_534280 |
| 1359 | Clu141870.con_1 | 427 | 3559.F17.GZ43_507492 |
| 1359 | Clu141870.con_1 | 512 | 3565.P03.GZ43_508046 |
| 1360 | Clu180092.con_1 | 849 | 3599.N20.GZ43_513183 |
| 1360 | Clu180092.con_1 | 266 | 3550.F06.GZ43_506164 |
| 1361 | Clu189355.con_1 | 1163 | 3664.D06.gz43_520606 |
| 1361 | Clu189355.con_1 | 1109 | 3661.E23.gz43_519727 |
| 1361 | Clu189355.con_1 | 1157 | 3663.N12.gz43_520328 |
| 1362 | Clu234667.con_1 | 309 | 3553.E08.GZ43_506579 |
| 1362 | Clu234667.con_1 | 747 | 3583.P22.GZ43_510672 |
| 1363 | Clu242901.con_1 | 332 | 3553.K05.GZ43_506537 |
| 1363 | Clu242901.con_1 | 561 | 3571.G22.GZ43_509127 |
| 1364 | Clu307985.con_1 | 254 | 3547.P18.GZ43_505982 |
| 1365 | Clu387610.con_1 | 552 | 3571.C08.GZ43_508899 |
| 1366 | Clu389995.con_2 | 523 | 3568.F06.GZ43_508486 |
| 1366 | Clu389995.con_2 | 776 | 3590.L08.GZ43_512221 |
| 1367 | Clu393948.con_1 | 473 | 3562.K08.GZ43_507737 |
| 1368 | Clu397313.con_1 | 880 | 3605.G13.gz43_513832 |
| 1368 | Clu397313.con_1 | 957 | 3617.F10.gz43_515319 |
| 1369 | Clu398439.con_1 | 224 | 3547.E04.GZ43_505747 |
| 1370 | Clu402150.con_1 | 214 | 3544.O20.GZ43_505629 |
| 1371 | Clu402789.con_1 | 416 | 3559.B04.GZ43_507280 |
| 1372 | Clu403488.con_1 | 768 | 3590.J01.GZ43_512107 |
| 1372 | Clu403488.con_1 | 917 | 3611.E07.gz43_514502 |
| 1373 | Clu413505.con_1 | 378 | 3556.D20.GZ43_507154 |
| 1374 | Clu418320.con_1 | 166 | 3541.I18.GZ43_505207 |
| 1374 | Clu418320.con_1 | 555 | 3571.E02.GZ43_508805 |
| 1374 | Clu418320.con_1 | 889 | 3605.N12.gz43_513823 |
| 1375 | Clu424678.con_1 | 139 | 3541.A23.GZ43_505279 |
| 1376 | Clu426138.con_1 | 177 | 3541.P22.GZ43_505278 |
| 1377 | Clu427904.con_1 | 696 | 3580.N14.GZ43_510158 |
| 1378 | Clu447151.con_1 | 1351 | 3762.M17.gz43_534257 |
| 1378 | Clu447151.con_1 | 606 | 3574.I07.GZ43_509273 |
| 1379 | Clu452564.con_1 | 203 | 3544.K16.GZ43_505561 |
| 1379 | Clu452564.con_1 | 1034 | 3632.M13.gz43_517517 |
| 1380 | Clu454826.con_1 | 220 | 3547.C17.GZ43_505953 |
| 1380 | Clu454826.con_1 | 430 | 3559.H24.GZ43_507606 |
| 1381 | Clu460499.con_1 | 330 | 3553.K02.GZ43_506489 |
| 1382 | Clu477110.con_1 | 235 | 3547.I17.GZ43_505959 |
| 1382 | Clu477110.con_1 | 490 | 3565.D19.GZ43_508290 |
| 1382 | Clu477110.con_1 | 587 | 3574.B24.GZ43_509538 |
| 1383 | Clu477708.con_1 | 514 | 3565.P22.GZ43_508350 |
| 1384 | Clu478212.con_1 | 317 | 3553.G21.GZ43_506789 |
| 1384 | Clu478212.con_1 | 246 | 3547.M02.GZ43_505723 |
| 1384 | Clu478212.con_1 | 230 | 3547.G22.GZ43_506037 |
| 1385 | Clu484459.con_1 | 1052 | 3635.M18.gz43_517981 |
| 1385 | Clu484459.con_1 | 1125 | 3662.C15.gz43_519981 |
| 1386 | Clu494499.con_1 | 197 | 3544.I15.GZ43_505543 |
| 1386 | Clu494499.con_1 | 572 | 3571.J14.GZ43_509002 |
| 1386 | Clu494499.con_1 | 725 | 3583.H15.GZ43_510552 |
| 1387 | Clu494890.con_1 | 300 | 3550.P23.GZ43_506446 |
| 1387 | Clu494890.con_1 | 605 | 3574.I02.GZ43_509193 |
| 1387 | Clu494890.con_1 | 791 | 3596.D17.GZ43_512741 |
| 1388 | Clu500919.con_1 | 1222 | 3754.B05.gz43_532902 |
| 1388 | Clu500919.con_1 | 226 | 3547.F10.GZ43_505844 |
| 1389 | Clu504038.con_1 | 935 | 3611.N09.gz43_514543 |
| 1389 | Clu504038.con_1 | 1112 | 3661.G20.gz43_519681 |
| 1390 | Clu504904.con_2 | 1167 | 3664.E23.gz43_520879 |
| 1391 | Clu505750.con_1 | 145 | 3538.G22.GZ43_504885 |
| 1391 | Clu505750.con_1 | 186 | 3544.E18.GZ43_505587 |
| 1392 | Clu517444.con_1 | 1021 | 3629.H10.gz43_517080 |
| 1392 | Clu517444.con_1 | 1134 | 3662.K03.gz43_519797 |
| 1393 | Clu528281.con_1 | 172 | 3541.M18.GZ43_505211 |
| 1393 | Clu528281.con_1 | 218 | 3547.A24.GZ43_506063 |
| 1394 | Clu529709.con_1 | 348 | 3553.K24.GZ43_506841 |
| 1394 | Clu529709.con_1 | 395 | 3556.K12.GZ43_507033 |
| 1395 | Clu532062.con_1 | 681 | 3580.K03.GZ43_509979 |
| 1395 | Clu532062.con_1 | 779 | 3590.M04.GZ43_512158 |
| 1396 | Clu542301.con_1 | 237 | 3547.J05.GZ43_505768 |
| 1396 | Clu542301.con_1 | 147 | 3538.H21.GZ43_504870 |
| 1397 | Clu542825.con_1 | 210 | 3544.N19.GZ43_505612 |
| 1397 | Clu542825.con_1 | 336 | 3538.A24.GZ43_504911 |
| 1398 | Clu548217.con_1 | 667 | 3580.G19.GZ43_510231 |
| 1398 | Clu548217.con_1 | 1175 | 3664.K16.gz43_520773 |
| 1399 | Clu548277.con_1 | 1006 | 3626.I20.gz43_516857 |
| 1400 | Clu554189.con_2 | 366 | 3553.P21.GZ43_506798 |
| 1401 | Clu555115.con_2 | 1185 | 3665.E20.gz43_521215 |
| 1402 | Clu555967.con_1 | 835 | 3599.F24.GZ43_513239 |
| 1402 | Clu555967.con_1 | 788 | 3596.D01.GZ43_512485 |
| 1403 | Clu557717.con_1 | 815 | 3596.O12.GZ43_512672 |
| 1403 | Clu557717.con_1 | 143 | 3538.G17.GZ43_504805 |
| 1403 | Clu557717.con_1 | 710 | 3583.B11.GZ43_510482 |
| 1404 | Clu566548.con_1 | 662 | 3580.E23.GZ43_510293 |
| 1405 | Clu567700.con_2 | 584 | 3574.B04.GZ43_509218 |
| 1406 | Clu573169.con_1 | 381 | 3556.E24.GZ43_507219 |
| 1406 | Clu573169.con_1 | 438 | 3559.L19.GZ43_507530 |
| 1407 | Clu585099.con_1 | 411 | 3556.O13.GZ43_507053 |
| 1407 | Clu585099.con_1 | 812 | 3596.N16.GZ43_512735 |
| 1407 | Clu585099.con_1 | 867 | 3602.G17.GZ43_513512 |
| 1408 | Clu585899.con_1 | 328 | 3553.J24.GZ43_506840 |
| 1408 | Clu585899.con_1 | 320 | 3553.H21.GZ43_506790 |
| 1409 | Clu609914.con_1 | 1181 | 3665.A23.gz43_521259 |
| 1409 | Clu609914.con_1 | 1114 | 3661.H24.gz43_519746 |
| 1410 | Clu613411.con_1 | 371 | 3556.B10.GZ43_506992 |
| 1410 | Clu613411.con_1 | 708 | 3583.B07.GZ43_510418 |
| 1411 | Clu621702.con_1 | 529 | 3568.G12.GZ43_508583 |
| 1411 | Clu621702.con_1 | 655 | 3580.D07.GZ43_510036 |
| 1412 | Clu637915.con_1 | 692 | 3580.M18.GZ43_510221 |
| 1412 | Clu637915.con_1 | 1220 | 3754.A16.gz43_533077 |
| 1413 | Clu640157.con_1 | 653 | 3580.C03.GZ43_509971 |
| 1414 | Clu640277.con_1 | 358 | 3553.N08.GZ43_506588 |
| 1414 | Clu640277.con_1 | 645 | 3577.P07.GZ43_509664 |
| 1415 | Clu645986.con_1 | 136 | 3541.A04.GZ43_504975 |
| 1415 | Clu645986.con_1 | 793 | 3596.E22.GZ43_512822 |
| 1416 | Clu647587.con_2 | 431 | 3559.I05.GZ43_507303 |
| 1417 | Clu652651.con_1 | 616 | 3574.N10.GZ43_509326 |
| 1417 | Clu652651.con_1 | 1043 | 3635.D07.gz43_517796 |
| 1417 | Clu652651.con_1 | 1173 | 3664.J12.gz43_520708 |
| 1418 | Clu653817.con_1 | 229 | 3547.G09.GZ43_505829 |
| 1418 | Clu653817.con_1 | 895 | 3608.E17.gz43_514278 |
| 1419 | Clu660907.con_1 | 173 | 3541.O04.GZ43_504989 |
| 1419 | Clu660907.con_1 | 1252 | 3754.N22.gz43_533186 |
| 1420 | Clu661802.con_1 | 844 | 3599.M04.GZ43_512926 |
| 1421 | Clu676665.con_1 | 596 | 3574.E02.GZ43_509189 |
| 1421 | Clu676665.con_1 | 209 | 3544.N12.GZ43_505500 |
| 1422 | Clu684638.con_1 | 1334 | 3762.D22.gz43_534328 |
| 1422 | Clu684638.con_1 | 1067 | 3643.F07.gz43_518566 |
| 1423 | Clu691512.con_1 | 977 | 3620.E23.gz43_516133 |
| 1423 | Clu691512.con_1 | 1122 | 3662.A13.gz43_519947 |
| 1423 | Clu691512.con_1 | 1348 | 3762.L18.gz43_534272 |
| 1424 | Clu700354.con_1 | 487 | 3565.C17.GZ43_508257 |
| 1425 | Clu708025.con_1 | 249 | 3547.M16.GZ43_505947 |
| 1425 | Clu708025.con_1 | 719 | 3583.G16.GZ43_510567 |
| 1426 | Clu710194.con_1 | 283 | 3550.K05.GZ43_506153 |
| 1426 | Clu710194.con_1 | 383 | 3556.G15.GZ43_507077 |
| 1426 | Clu710194.con_1 | 519 | 3568.C22.GZ43_508739 |
| 1427 | Clu733840.con_1 | 1056 | 3635.P18.gz43_517984 |
| 1427 | Clu733840.con_1 | 1131 | 3662.J08.gz43_519876 |
| 1427 | Clu733840.con_1 | 1187 | 3665.K01.gz43_520917 |
| 1428 | Clu734568.con_1 | 857 | 3602.B22.GZ43_513587 |
| 1428 | Clu734568.con_1 | 886 | 3605.M17.gz43_513902 |
| 1429 | Clu742101.con_1 | 477 | 3562.O18.GZ43_507901 |
| 1429 | Clu742101.con_1 | 1201 | 3666.A24.gz43_521659 |
| 1430 | Clu747805.con_1 | 744 | 3583.O17.GZ43_510591 |
| 1430 | Clu747805.con_1 | 817 | 3596.P04.GZ43_512545 |
| 1431 | Clu761460.con_1 | 947 | 3614.K22.gz43_515132 |
| 1431 | Clu761460.con_1 | 1045 | 3635.F06.gz43_517782 |
| 1431 | Clu761460.con_1 | 1166 | 3664.E18.gz43_520799 |
| 1432 | Clu765500.con_1 | 1210 | 3666.I12.gz43_521475 |
| 1433 | Clu770834.con_1 | 799 | 3596.H22.GZ43_512825 |
| 1433 | Clu770834.con_1 | 1330 | 3762.D03.gz43_534024 |
| 1434 | Clu774520.con_1 | 363 | 3553.P05.GZ43_506542 |
| 1434 | Clu774520.con_1 | 380 | 3556.E13.GZ43_507043 |
| 1435 | Clu777670.con_1 | 714 | 3583.E13.GZ43_510517 |
| 1435 | Clu777670.con_1 | 803 | 3596.J13.GZ43_512683 |
| 1436 | Clu812031.con_1 | 162 | 3541.G17.GZ43_505189 |
| 1436 | Clu812031.con_1 | 184 | 3544.B18.GZ43_505584 |
| 1437 | Clu821536.con_1 | 278 | 3550.I03.GZ43_506119 |
| 1437 | Clu821536.con_1 | 746 | 3583.P19.GZ43_510624 |
| 1438 | Clu823271.con_1 | 386 | 3556.H12.GZ43_507030 |
| 1439 | Clu845354.con_1 | 892 | 3608.B12.gz43_514195 |
| 1440 | Clu846056.con_1 | 357 | 3553.N07.GZ43_506572 |
| 1440 | Clu846056.con_1 | 1060 | 3638.H07.gz43_518184 |
| 1441 | Clu854573.con_1 | 1035 | 3632.M19.gz43_517613 |
| 1441 | Clu854573.con_1 | 841 | 3599.K23.GZ43_513228 |
| 1442 | Clu863768.con_1 | 417 | 3559.B06.GZ43_507312 |
| 1443 | Clu869453.con_1 | 784 | 3590.O08.GZ43_512224 |
| 1444 | Clu875978.con_2 | 951 | 3614.O07.gz43_514896 |
| 1444 | Clu875978.con_2 | 961 | 3617.L21.gz43_515501 |
| 1445 | Clu893981.con_1 | 277 | 3550.H23.GZ43_506438 |
| 1445 | Clu893981.con_1 | 474 | 3562.L12.GZ43_507802 |
| 1446 | Clu945247.con_1 | 622 | 3577.A18.GZ43_509825 |
| 1446 | Clu945247.con_1 | 158 | 3538.O07.GZ43_504653 |
| 1446 | Clu945247.con_1 | 436 | 3559.L01.GZ43_507242 |
| 1447 | Clu947168.con_1 | 175 | 3541.O23.GZ43_505293 |
| 1448 | Clu961757.con_1 | 698 | 3580.N23.GZ43_510302 |
| 1448 | Clu961757.con_1 | 1248 | 3754.K20.gz43_533151 |
| 1449 | Clu968647.con_1 | 1104 | 3646.P17.gz43_519120 |
| 1449 | Clu968647.con_1 | 1343 | 3762.I07.gz43_534093 |
| 1450 | Clu970165.con_1 | 299 | 3550.P18.GZ43_506366 |
| 1450 | Clu970165.con_1 | 369 | 3556.B06.GZ43_506928 |
| 1450 | Clu970165.con_1 | 465 | 3562.H12.GZ43_507798 |
| 1451 | Clu991366.con_1 | 280 | 3550.I21.GZ43_506407 |
| 1451 | Clu991366.con_1 | 445 | 3559.O05.GZ43_507309 |
| 1451 | Clu991366.con_1 | 657 | 3580.E02.GZ43_509957 |
| 1452 | Clu1014734.con_1 | 344 | 3538.E15.GZ43_504771 |
| 1452 | Clu1014734.con_1 | 770 | 3590.J18.GZ43_512379 |
| 1452 | Clu1014734.con_1 | 1345 | 3762.J18.gz43_534270 |
| 1453 | Clu1037887.con_1 | 178 | 3544.A09.GZ43_505439 |
| 1453 | Clu1037887.con_1 | 272 | 3550.G10.GZ43_506229 |
| 1454 | Clu1052399.con_1 | 507 | 3565.N19.GZ43_508300 |
| 1455 | Clu1052466.con_1 | 264 | 3550.E02.GZ43_506099 |
| 1455 | Clu1052466.con_1 | 528 | 3568.G10.GZ43_508551 |
| 1456 | Clu1053121.con_1 | 619 | 3574.P07.GZ43_509280 |
| 1456 | Clu1053121.con_1 | 688 | 3580.L17.GZ43_510204 |
| 1457 | Clu1053514.con_1 | 1053 | 3635.O01.gz43_517711 |
| 1457 | Clu1053514.con_1 | 1155 | 3663.N09.gz43_520280 |
| 1458 | Clu1053747.con_1 | 890 | 3605.N16.gz43_513887 |
| 1458 | Clu1053747.con_1 | 932 | 3611.M18.gz43_514686 |
| 1459 | Clu1053799.con_1 | 467 | 3562.I02.GZ43_507639 |
| 1460 | Clu1053854.con_1 | 898 | 3608.G09.gz43_514152 |
| 1460 | Clu1053854.con_1 | 1039 | 3632.P07.gz43_517424 |
| 1461 | Clu1054038.con_1 | 271 | 3550.G08.GZ43_506197 |
| 1461 | Clu1054038.con_1 | 288 | 3550.L23.GZ43_506442 |
| 1461 | Clu1054038.con_1 | 419 | 3559.B10.GZ43_507376 |
| 1462 | Clu1054069.con_1 | 741 | 3583.N09.GZ43_510462 |
| 1463 | Clu1054074.con_1 | 231 | 3547.H12.GZ43_505878 |
| 1464 | Clu1054807.con_1 | 221 | 3547.C23.GZ43_506049 |
| 1464 | Clu1054807.con_1 | 1256 | 3754.P17.gz43_533108 |
| 1465 | Clu1054813.con_1 | 392 | 3556.J14.GZ43_507064 |
| 1465 | Clu1054813.con_1 | 1325 | 3762.A20.gz43_534293 |
| 1465 | Clu1054813.con_1 | 324 | 3553.J14.GZ43_506680 |
| 1466 | Clu1054884.con_1 | 581 | 3571.O08.GZ43_508911 |
| 1466 | Clu1054884.con_1 | 571 | 3571.J09.GZ43_508922 |
| 1467 | Clu1055018.con_1 | 483 | 3565.B13.GZ43_508192 |
| 1468 | Clu1055063.con_1 | 504 | 3565.M20.GZ43_508315 |
| 1469 | Clu1055089.con_1 | 326 | 3553.J17.GZ43_506728 |
| 1469 | Clu1055089.con_1 | 570 | 3571.J08.GZ43_508906 |
| 1469 | Clu1055089.con_1 | 763 | 3590.H06.GZ43_512185 |
| 1470 | Clu1055256.con_1 | 945 | 3614.H22.gz43_515129 |
| 1470 | Clu1055256.con_1 | 1037 | 3632.N21.gz43_517646 |
| 1470 | Clu1055256.con_1 | 1156 | 3663.N10.gz43_520296 |
| 1471 | Clu1055326.con_1 | 1030 | 3632.G01.gz43_517319 |
| 1471 | Clu1055326.con_1 | 1176 | 3664.K19.gz43_520821 |
| 1472 | Clu1056369.con_1 | 239 | 3547.J20.GZ43_506008 |
| 1472 | Clu1056369.con_1 | 372 | 3556.B14.GZ43_507056 |
| 1472 | Clu1056369.con_1 | 1341 | 3762.G18.gz43_534267 |
| 1473 | Clu1059332.con_1 | 583 | 3574.B01.GZ43_509170 |
| 1474 | Clu1059445.con_1 | 245 | 3547.L22.GZ43_506042 |
| 1475 | Clu1060021.con_1 | 232 | 3547.H14.GZ43_505910 |
| 1475 | Clu1060021.con_1 | 253 | 3547.O14.GZ43_505917 |
| 1476 | Clu1061206.con_1 | 973 | 3620.E12.gz43_515957 |
| 1476 | Clu1061206.con_1 | 1088 | 3646.C06.gz43_518931 |
| 1476 | Clu1061206.con_1 | 1186 | 3665.H20.gz43_521218 |
| 1477 | Clu1062537.con_1 | 286 | 3550.L16.GZ43_506330 |
| 1477 | Clu1062537.con_1 | 449 | 3559.P15.GZ43_507470 |
| 1478 | Clu1064975.con_1 | 325 | 3553.J16.GZ43_506712 |
| 1478 | Clu1064975.con_1 | 713 | 3583.E11.GZ43_510485 |
| 1478 | Clu1064975.con_1 | 265 | 3550.E06.GZ43_506163 |
| 1479 | Clu1065531.con_1 | 337 | 3538.B01.GZ43_504544 |
| 1480 | Clu1066041.con_1 | 508 | 3565.O02.GZ43_508029 |
| 1480 | Clu1066041.con_1 | 842 | 3599.L04.GZ43_512925 |
| 1481 | Clu1069578.con_1 | 1171 | 3664.H15.gz43_520754 |
| 1482 | Clu1069632.con_1 | 550 | 3571.B13.GZ43_508978 |
| 1482 | Clu1069632.con_1 | 551 | 3571.B22.GZ43_509122 |
| 1483 | Clu1073767.con_1 | 193 | 3544.H03.GZ43_505350 |
| 1483 | Clu1073767.con_1 | 1245 | 3754.J24.gz43_533214 |
| 1484 | Clu1074160.con_1 | 296 | 3550.O17.GZ43_506349 |
| 1484 | Clu1074160.con_1 | 672 | 3580.H22.GZ43_510280 |
| 1485 | Clu1076930.con_1 | 635 | 3577.J04.GZ43_509610 |
| 1485 | Clu1076930.con_1 | 983 | 3620.K24.gz43_516155 |
| 1486 | Clu1077033.con_1 | 414 | 3559.A20.GZ43_507535 |
| 1487 | Clu1079196.con_1 | 915 | 3611.B16.gz43_514643 |
| 1487 | Clu1079196.con_1 | 943 | 3614.G20.gz43_515096 |
| 1488 | Clu1079863.con_1 | 170 | 3541.M02.GZ43_504955 |
| 1488 | Clu1079863.con_1 | 547 | 3571.A11.GZ43_508945 |
| 1488 | Clu1079863.con_1 | 931 | 3611.L22.gz43_514749 |
| 1489 | Clu1085638.con_1 | 1232 | 3754.F11.gz43_533002 |
| 1489 | Clu1085638.con_1 | 334 | 3553.K15.GZ43_506697 |
| 1489 | Clu1085638.con_1 | 858 | 3602.C24.GZ43_513620 |
| 1490 | Clu1085645.con_1 | 715 | 3583.E15.GZ43_510549 |
| 1491 | Clu1088847.con_1 | 1116 | 3661.J15.gz43_519604 |
| 1491 | Clu1088847.con_1 | 1204 | 3666.D02.gz43_521310 |
| 1491 | Clu1088847.con_1 | 1059 | 3638.F15.gz43_518310 |
| 1492 | Clu1088930.con_1 | 999 | 3623.N23.gz43_516526 |
| 1493 | Clu1108332.con_2 | 856 | 3602.B21.GZ43_513571 |
| 1494 | Clu1110143.con_1 | 365 | 3553.P18.GZ43_506750 |
| 1494 | Clu1110143.con_1 | 537 | 3568.M03.GZ43_508445 |
| 1495 | Clu1116087.con_1 | 409 | 3556.N21.GZ43_507180 |
| 1495 | Clu1116087.con_1 | 488 | 3565.D14.GZ43_508210 |
| 1495 | Clu1116087.con_1 | 557 | 3571.E16.GZ43_509029 |
| 1496 | Clu1131409.con_1 | 875 | 3602.N03.GZ43_513295 |
| 1496 | Clu1131409.con_1 | 1135 | 3662.L05.gz43_519830 |
| 1497 | Clu1132413.con_1 | 654 | 3580.C05.GZ43_510003 |
| 1497 | Clu1132413.con_1 | 879 | 3605.E19.gz43_513926 |
| 1498 | Clu1136803.con_1 | 171 | 3541.M07.GZ43_505035 |
| 1498 | Clu1136803.con_1 | 219 | 3547.C05.GZ43_505761 |
| 1499 | Clu1138419.con_1 | 250 | 3547.N06.GZ43_505788 |
| 1500 | Clu1138593.con_1 | 198 | 3544.I20.GZ43_505623 |
| 1500 | Clu1138593.con_1 | 374 | 3556.C15.GZ43_507073 |
| 1500 | Clu1138593.con_1 | 377 | 3556.D15.GZ43_507074 |
| 1501 | Clu1139522.con_2 | 650 | 3580.A14.GZ43_510145 |
| 1501 | Clu1139522.con_2 | 1328 | 3762.C20.gz43_534295 |
| 1502 | Clu1139691.con_1 | 199 | 3544.J04.GZ43_505368 |
| 1503 | Clu1142019.con_1 | 1209 | 3666.G12.gz43_521473 |
| 1504 | Clu1171518.con_2 | 494 | 3565.G22.GZ43_508341 |
| 1505 | Clu1176182.con_1 | 566 | 3571.H16.GZ43_509032 |
| 1506 | Clu1182447.con_1 | 1094 | 3646.H16.gz43_519096 |
| 1507 | Clu1183079.con_1 | 923 | 3611.I04.gz43_514458 |
| 1508 | Clu1184134.con_1 | 789 | 3596.D07.GZ43_512581 |
| 1509 | Clu1189027.con_1 | 549 | 3571.A22.GZ43_509121 |
| 1509 | Clu1189027.con_1 | 800 | 3596.I06.GZ43_512570 |
| 1509 | Clu1189027.con_1 | 801 | 3596.I16.GZ43_512730 |
| 1510 | Clu1191547.con_1 | 164 | 3541.I15.GZ43_505159 |
| 1510 | Clu1191547.con_1 | 1323 | 3762.A16.gz43_534229 |
| 1511 | Clu1193236.con_1 | 595 | 3574.D12.GZ43_509348 |
| 1511 | Clu1193236.con_1 | 569 | 3571.J07.GZ43_508890 |
| 1511 | Clu1193236.con_1 | 559 | 3571.F16.GZ43_509030 |
| 1512 | Clu1210953.con_1 | 533 | 3568.J22.GZ43_508746 |
| 1512 | Clu1210953.con_1 | 434 | 3559.K16.GZ43_507481 |
| 1513 | Clu1211899.con_1 | 329 | 3553.K01.GZ43_506473 |
| 1513 | Clu1211899.con_1 | 385 | 3556.H02.GZ43_506870 |
| 1513 | Clu1211899.con_1 | 390 | 3556.J05.GZ43_506920 |
| 1514 | Clu1218793.con_1 | 511 | 3565.O15.GZ43_508237 |
| 1514 | Clu1218793.con_1 | 1242 | 3754.J05.gz43_532910 |
| 1515 | Clu1223271.con_1 | 340 | 3538.C02.GZ43_504561 |
| 1515 | Clu1223271.con_1 | 604 | 3574.H07.GZ43_509272 |
| 1516 | Clu1223477.con_1 | 289 | 3550.M21.GZ43_506411 |
| 1517 | Clu1223907.con_1 | 652 | 3580.C01.GZ43_509939 |
| 1518 | Clu1223938.con_1 | 244 | 3547.L16.GZ43_505946 |
| 1519 | Clu1223948.con_1 | 176 | 3541.P05.GZ43_505006 |
| 1520 | Clu1224039.con_1 | 236 | 3547.I20.GZ43_506007 |
| 1520 | Clu1224039.con_1 | 397 | 3556.K17.GZ43_507113 |
| 1521 | Clu1224226.con_1 | 516 | 3568.A10.GZ43_508545 |
| 1521 | Clu1224226.con_1 | 608 | 3574.J14.GZ43_509386 |
| 1522 | Clu1224379.con_1 | 262 | 3550.D16.GZ43_506322 |
| 1522 | Clu1224379.con_1 | 402 | 3556.M02.GZ43_506875 |
| 1523 | Clu1224422.con_1 | 370 | 3556.B09.GZ43_506976 |
| 1523 | Clu1224422.con_1 | 394 | 3556.K04.GZ43_506905 |
| 1524 | Clu1224547.con_1 | 626 | 3577.E19.GZ43_509845 |
| 1525 | Clu1224704.con_1 | 756 | 3590.E08.GZ43_512214 |
| 1526 | Clu1224752.con_1 | 532 | 3568.J10.GZ43_508554 |
| 1526 | Clu1224752.con_1 | 194 | 3544.H15.GZ43_505542 |
| 1527 | Clu1224881.con_2 | 475 | 3562.N24.GZ43_507996 |
| 1527 | Clu1224881.con_2 | 1111 | 3661.G16.gz43_519617 |
| 1528 | Clu1225500.con_1 | 270 | 3550.G02.GZ43_506101 |
| 1528 | Clu1225500.con_1 | 548 | 3571.A14.GZ43_508993 |
| 1528 | Clu1225500.con_1 | 1066 | 3643.E24.gz43_518837 |
| 1529 | Clu1225595.con_1 | 455 | 3562.C23.GZ43_507969 |
| 1530 | Clu1225719.con_2 | 297 | 3550.O18.GZ43_506365 |
| 1530 | Clu1225719.con_2 | 1327 | 3762.B15.gz43_534214 |
| 1531 | Clu1225734.con_1 | 907 | 3608.L14.gz43_514237 |
| 1532 | Clu1226064.con_1 | 227 | 3547.F20.GZ43_506004 |
| 1533 | Clu1226304.con_1 | 783 | 3590.N21.GZ43_512431 |
| 1534 | Clu1226413.con_1 | 611 | 3574.K20.GZ43_509483 |
| 1534 | Clu1226413.con_1 | 480 | 3562.P23.GZ43_507982 |
| 1535 | Clu1226932.con_1 | 345 | 3538.F02.GZ43_504564 |
| 1535 | Clu1226932.con_1 | 1255 | 3754.P13.gz43_533044 |
| 1536 | Clu1227623.con_1 | 834 | 3599.F17.GZ43_513127 |
| 1536 | Clu1227623.con_1 | 967 | 3617.N19.gz43_515471 |
| 1537 | Clu1227781.con_1 | 349 | 3553.L02.GZ43_506490 |
| 1537 | Clu1227781.con_1 | 1014 | 3626.P14.gz43_516768 |
| 1538 | Clu1227862.con_2 | 656 | 3580.D22.GZ43_510276 |
| 1538 | Clu1227862.con_2 | 661 | 3580.E21.GZ43_510261 |
| 1539 | Clu1227912.con_1 | 1241 | 3754.J01.gz43_532846 |
| 1539 | Clu1227912.con_1 | 426 | 3559.F07.GZ43_507332 |
| 1539 | Clu1227912.con_1 | 423 | 3559.E06.GZ43_507315 |
| 1540 | Clu1227968.con_1 | 1168 | 3664.E24.gz43_520895 |
| 1540 | Clu1227968.con_1 | 361 | 3553.O23.GZ43_506829 |
| 1541 | Clu1228277.con_1 | 854 | 3602.A09.GZ43_513378 |
| 1542 | Clu1230257.con_1 | 1068 | 3643.G20.gz43_518775 |
| 1542 | Clu1230257.con_1 | 1086 | 3646.A13.gz43_519041 |
| 1543 | Clu1245188.con_1 | 400 | 3556.L16.GZ43_507098 |
| 1543 | Clu1245188.con_1 | 1142 | 3663.E04.gz43_520191 |
| 1544 | Clu1250373.con_1 | 422 | 3559.D21.GZ43_507554 |
| 1544 | Clu1250373.con_1 | 1141 | 3663.C19.gz43_520429 |
| 1544 | Clu1250373.con_1 | 1203 | 3666.C18.gz43_521565 |
| 1545 | Clu1256564.con_2 | 541 | 3568.P04.GZ43_508464 |
| 1545 | Clu1256564.con_2 | 1178 | 3664.O22.gz43_520873 |
| 1546 | Clu1259069.con_2 | 1349 | 3762.L20.gz43_534304 |
| 1547 | Clu1274736.con_1 | 1198 | 3665.P13.gz43_521114 |
| 1547 | Clu1274736.con_1 | 1216 | 3666.N06.gz43_521384 |
| 1548 | Clu1283437.con_2 | 213 | 3544.O15.GZ43_505549 |
| 1548 | Clu1283437.con_2 | 981 | 3620.J18.gz43_516058 |
| 1549 | Clu1292262.con_1 | 883 | 3605.I19.gz43_513930 |
| 1549 | Clu1292262.con_1 | 968 | 3617.P11.gz43_515345 |
| 1549 | Clu1292262.con_1 | 1117 | 3661.K22.gz43_519717 |
| 1550 | Clu1292281.con_1 | 881 | 3605.H10.gz43_513785 |
| 1550 | Clu1292281.con_1 | 1189 | 3665.M21.gz43_521239 |
| 1551 | Clu1292289.con_1 | 938 | 3614.C18.gz43_515060 |
| 1551 | Clu1292289.con_1 | 1098 | 3646.K14.gz43_519067 |
| 1552 | Clu1292413.con_1 | 1016 | 3629.B14.gz43_517138 |
| 1552 | Clu1292413.con_1 | 1215 | 3666.M16.gz43_521543 |
| 1552 | Clu1292413.con_1 | 976 | 3620.E19.gz43_516069 |
| 1553 | Clu1292423.con_1 | 939 | 3614.D14.gz43_514997 |
| 1553 | Clu1292423.con_1 | 965 | 3617.N10.gz43_515327 |
| 1554 | Clu1292436.con_1 | 958 | 3617.H16.gz43_515417 |
| 1554 | Clu1292436.con_1 | 1074 | 3643.I24.gz43_518841 |
| 1555 | Clu1292439.con_1 | 946 | 3614.J07.gz43_514891 |
| 1555 | Clu1292439.con_1 | 1172 | 3664.H22.gz43_520866 |
| 1556 | Clu1292582.con_1 | 1199 | 3666.A07.gz43_521387 |
| 1556 | Clu1292582.con_1 | 1040 | 3635.A06.gz43_517777 |
| 1557 | Clu1292600.con_1 | 1115 | 3661.I22.gz43_519715 |
| 1557 | Clu1292600.con_1 | 919 | 3611.E20.gz43_514710 |
| 1557 | Clu1292600.con_1 | 1064 | 3638.N05.gz43_518158 |
| 1558 | Clu1292932.con_1 | 1154 | 3663.M24.gz43_520519 |
| 1558 | Clu1292932.con_1 | 1170 | 3664.G20.gz43_520833 |
| 1559 | Clu1292996.con_1 | 1018 | 3629.E01.gz43_516933 |
| 1559 | Clu1292996.con_1 | 1036 | 3632.N13.gz43_517518 |
| 1559 | Clu1292996.con_1 | 1161 | 3664.A11.gz43_520683 |
| 1560 | Clu1293946.con_1 | 1177 | 3664.L21.gz43_520854 |
| 1560 | Clu1293946.con_1 | 1205 | 3666.D11.gz43_521454 |
| 1561 | Clu1293972.con_1 | 1099 | 3646.L17.gz43_519116 |
| 1561 | Clu1293972.con_1 | 954 | 3614.P16.gz43_515041 |
| 1561 | Clu1293972.con_1 | 1010 | 3626.N07.gz43_516654 |

### Example 19

### Additional Gene Characterization

Sequences of the polynucleotides of SEQ ID NOS: 134-1352 were used as a query sequence in a TeraBLASTN search of the DoubleTwist Human Genome Sequence Database (DoubleTwist, Inc., Oakland, Calif.), which contains all the human genomic sequences that have been assembled into a contiguous model of the human genome. Predicted cDNA and protein sequences were obtained where a polynucleotide of the invention was homologous to a predicted full-length gene sequence. Alternatively, a sequence of a contig or consensus sequence described herein could be used directly as a query sequence in a TeraBLASTN search of the DoubleTwist Human Genome Sequence Database.

The final results of the search provided the predicted cDNA sequences listed as SEQ ID NOS: 1562-1618 in the accompanying Sequence Listing and summarized in Table 14 (inserted prior to claims), and the predicted protein sequences listed as SEQ ID SEQ ID NOS:1619-1675 in the accompanying Sequence Listing and summarized in Table 15 (inserted prior to claims). Specifically, Table 14 provides: 1) the SEQ ID NO (“SEQ ID”) assigned to each cDNA sequence for use in the present specification; 2) the cDNA sequence name (“cDNA SEQ NAME”) used as an internal identifier of the sequence; 3) the chromosome (“CHROM”) containing the gene corresponding to the cDNA sequence; and 4) the exon (“EXON”) of the gene corresponding to the cDNA sequence to which the polynucleotide of SEQ ID NOS: 134-1352 maps. Table 15 provides: 1) the SEQ ID NO (“SEQ ID”) assigned to each protein sequence for use in the present specification; 2) the protein sequence name (“PROTEIN SEQ NAME”) used as an internal identifier of the sequence; 3) the chromosome (“CHROM”) containing the gene corresponding to the cDNA sequence; and 4) the exon (“EXON”) of the gene corresponding to the cDNA and protein sequence to which the polynucleotide of SEQ ID NOS: 134-1352 maps.

TABLE 14

| SEQ ID | cDNA SEQ NAME | CHROM | EXON |
|---|---|---|---|
| 1562 | NTN_004511S11.3_4 | Chr(1) | Exons(14) |
| 1563 | NTN_004754S1.3_4 | Chr(1) | Exons(5) |
| 1564 | NTN_005530S2.3_3 | Chr(3) | Exons(5) |
| 1565 | NTN_005530S2.3_4 | Chr(3) | Exons(6) |
| 1566 | NTN_005564S1.3_3 | Chr(3) | Exons(2) |
| 1567 | NTN_005635S2.3_1 | Chr(3) | Exons(10) |
| 1568 | NTN_005962S3.3_4 | Chr(3) | Exons(9) |
| 1569 | NTN_006051S4.3_2 | Chr(4) | Exons(17) |
| 1570 | NTN_006051S5.3_5 | Chr(4) | Exons(6) |
| 1571 | NTN_007011S7.3_9 | Chr(5) | Exons(16) |
| 1572 | NTN_007122S8.3_8 | Chr(6) | Exons(12) |
| 1573 | NTN_007592S2.3_10 | Chr(6) | Exons(3) |
| 1574 | NTN_007592S3.3_5 | Chr(6) | Exons(1) |
| 1575 | NTN_007844S7.3_3 | Chr(7) | Exons(3) |
| 1576 | NTN_007867S7.3_3 | Chr(7) | Exons(15) |
| 1577 | NTN_007867S8.3_1 | Chr(7) | Exons(14) |
| 1578 | NTN_008059S2.3_3 | Chr(8) | Exons(8) |
| 1579 | NTN_008338S4.3_6 | Chr(9) | Exons(4) |
| 1580 | NTN_008470S7.3_1 | Chr(9) | Exons(13) |
| 1581 | NTN_008627S7.3_5 | Chr(10) | Exons(13) |
| 1582 | NTN_008769S8.3_1 | Chr(10) | Exons(7) |
| 1583 | NTN_008858S2.3_2 | Chr(10) | Exons(5) |
| 1584 | NTN_009296S1.3_1 | Chr(11) | Exons(6) |
| 1585 | NTN_009296S3.3_2 | Chr(11) | Exons(3) |
| 1586 | NTN_009526S2.3_3 | Chr(12) | Exons(11) |
| 1587 | NTN_009526S2.3_5 | Chr(12) | Exons(11) |
| 1588 | NTN_009848S8.3_5 | Chr(13) | Exons(3) |
| 1589 | NTN_009866S24.3_5 | Chr(13) | Exons(5) |
| 1590 | NTN_010018S2.3_5 | Chr(14) | Exons(12) |
| 1591 | NTN_010164S3.3_8 | Chr(14) | Exons(3) |
| 1592 | NTN_010289S6.3_5 | Chr(15) | Exons(2) |
| 1593 | NTN_010663S1.3_1 | Chr(17) | Exons(4) |
| 1594 | NTN_010757S4.3_2 | Chr(17) | Exons(25) |
| 1595 | NTN_011059S6.3_4 | Chr(18) | Exons(2) |
| 1596 | NTN_011130S2.3_3 | Chr(19) | Exons(6) |
| 1597 | NTN_011266S2.2_3 | Chr(19) | Exons(1) |
| 1598 | NTN_011361S6.3_7 | Chr(20) | Exons(6) |
| 1599 | NTN_011430S6.3_6 | Chr(20) | Exons(22) |
| 1600 | NTN_011512S51.3_3 | Chr(21) | Exons(11) |
| 1601 | NTN_017582S2.3_6 | Chr(9) | Exons(2) |
| 1602 | NTN_019508S1.3_6 | Chr(10) | Exons(8) |
| 1603 | NTN_019721S6.3_1 | Chr(Y) | Exons(5) |
| 1604 | NTN_022448S1.3_2 | Chr(3) | Exons(11) |
| 1605 | NTN_022456S1.3_2 | Chr(3) | Exons(6) |
| 1606 | NTN_022526S1.3_2 | Chr(3) | Exons(10) |
| 1607 | NTN_022807S2.3_1 | Chr(4) | Exons(2) |
| 1608 | NTN_022948S1.3_1 | Chr(4) | Exons(4) |
| 1609 | NTN_022948S1.3_5 | Chr(4) | Exons(4) |
| 1610 | NTN_023142S2.3_4 | Chr(5) | Exons(2) |
| 1611 | NTN_023860S1.3_1 | Chr(8) | Exons(3) |
| 1612 | NTN_024001S1.3_4 | Chr(9) | Exons(6) |
| 1613 | NTN_024871S1.3_7 | Chr(17) | Exons(4) |
| 1614 | NTN_024882S1.3_3 | Chr(17) | Exons(8) |
| 1615 | NTN_025842S13.2_1 | Chr(11) | Exons(10) |
| 1616 | NTN_025864S1.1_1 | Chr(12) | Exons(3) |
| 1617 | NTN_025907S4.2_3 | Chr(17) | Exons(5) |
| 1618 | NTN_026331S1.1_1 | Chr(7) | Exons(14) |

TABLE 15

| SEQ ID | PROTEIN SEQ NAME | CHROM | EXON |
|---|---|---|---|
| 1619 | NTP_004511S11.3_4 | Chr(1) | Exons(14) |
| 1620 | NTP_004754S1.3_4 | Chr(1) | Exons(5) |
| 1621 | NTP_005530S2.3_3 | Chr(3) | Exons(5) |
| 1622 | NTP_005530S2.3_4 | Chr(3) | Exons(6) |
| 1623 | NTP_005564S1.3_3 | Chr(3) | Exons(2) |
| 1624 | NTP_005635S2.3_1 | Chr(3) | Exons(10) |
| 1625 | NTP_005962S3.3_4 | Chr(3) | Exons(9) |
| 1626 | NTP_006051S4.3_2 | Chr(4) | Exons(17) |
| 1627 | NTP_006051S5.3_5 | Chr(4) | Exons(6) |
| 1628 | NTP_007011S7.3_9 | Chr(5) | Exons(16) |
| 1629 | NTP_007122S8.3_8 | Chr(6) | Exons(12) |
| 1630 | NTP_007592S2.3_10 | Chr(6) | Exons(3) |
| 1631 | NTP_007592S3.3_5 | Chr(6) | Exons(1) |
| 1632 | NTP_007844S7.3_3 | Chr(7) | Exons(3) |
| 1633 | NTP_007867S7.3_3 | Chr(7) | Exons(15) |
| 1634 | NTP_007867S8.3_1 | Chr(7) | Exons(14) |
| 1635 | NTP_008059S2.3_3 | Chr(8) | Exons(8) |
| 1636 | NTP_008338S4.3_6 | Chr(9) | Exons(4) |
| 1637 | NTP_008470S7.3_1 | Chr(9) | Exons(13) |
| 1638 | NTP_008627S7.3_5 | Chr(10) | Exons(13) |
| 1639 | NTP_008769S8.3_1 | Chr(10) | Exons(7) |
| 1640 | NTP_008858S2.3_2 | Chr(10) | Exons(5) |
| 1641 | NTP_009296S1.3_1 | Chr(11) | Exons(6) |
| 1642 | NTP_009296S3.3_2 | Chr(11) | Exons(3) |
| 1643 | NTP_009526S2.3_3 | Chr(12) | Exons(11) |
| 1644 | NTP_009526S2.3_5 | Chr(12) | Exons(11) |
| 1645 | NTP_009848S8.3_5 | Chr(13) | Exons(3) |
| 1646 | NTP_009866S24.3_5 | Chr(13) | Exons(5) |
| 1647 | NTP_010018S2.3_5 | Chr(14) | Exons(12) |
| 1648 | NTP_010164S3.3_8 | Chr(14) | Exons(3) |
| 1649 | NTP_010289S6.3_5 | Chr(15) | Exons(2) |
| 1650 | NTP_010663S1.3_1 | Chr(17) | Exons(4) |
| 1651 | NTP_010757S4.3_2 | Chr(17) | Exons(25) |
| 1652 | NTP_011059S6.3_4 | Chr(18) | Exons(2) |
| 1653 | NTP_011130S2.3_3 | Chr(19) | Exons(6) |
| 1654 | NTP_011266S2.2_3 | Chr(19) | Exons(1) |
| 1655 | NTP_011361S6.3_7 | Chr(20) | Exons(6) |
| 1656 | NTP_011430S6.3_6 | Chr(20) | Exons(22) |
| 1657 | NTP_011512S51.3_3 | Chr(21) | Exons(11) |
| 1658 | NTP_017582S2.3_6 | Chr(9) | Exons(2) |
| 1659 | NTP_019508S1.3_6 | Chr(10) | Exons(8) |
| 1660 | NTP_019721S6.3_1 | Chr(Y) | Exons(5) |
| 1661 | NTP_022448S1.3_2 | Chr(3) | Exons(11) |
| 1662 | NTP_022456S1.3_2 | Chr(3) | Exons(6) |
| 1663 | NTP_022526S1.3_2 | Chr(3) | Exons(10) |
| 1664 | NTP_022807S2.3_1 | Chr(4) | Exons(2) |
| 1665 | NTP_022948S1.3_1 | Chr(4) | Exons(4) |
| 1666 | NTP_022948S1.3_5 | Chr(4) | Exons(4) |
| 1667 | NTP_023142S2.3_4 | Chr(5) | Exons(2) |
| 1668 | NTP_023860S1.3_1 | Chr(8) | Exons(3) |
| 1669 | NTP_024001S1.3_4 | Chr(9) | Exons(6) |
| 1670 | NTP_024871S1.3_7 | Chr(17) | Exons(4) |
| 1671 | NTP_024882S1.3_3 | Chr(17) | Exons(8) |
| 1672 | NTP_025842S13.2_1 | Chr(11) | Exons(10) |
| 1673 | NTP_025864S1.1_1 | Chr(12) | Exons(3) |
| 1674 | NTP_025907S4.2_3 | Chr(17) | Exons(5) |
| 1675 | NTP_026331S1.1_1 | Chr(7) | Exons(14) |

A correlation between the polynucleotide used as a query sequence as described above and the corresponding predicted cDNA and protein sequences is contained in Table 16. Specifically Table 16 provides: 1) the SEQ ID NO of the cDNA (“cDNA SEQ ID”); 2) the cDNA sequence name (“cDNA SEQ NAME”) used as an internal identifier of sequence; 3) the SEQ ID NO of the protein (“PROTEIN SEQ ID”) encoded by the cDNA sequence 4) the sequence name of the protein (“PROTEIN SEQ NAME”) encoded by the cDNA sequence; 5) the SEQ ID NO of the polynucleotide (“POLYNTD SEQ ID”) of SEQ ID NOS: 134-1352 that maps to the cDNA and protein; and 6) the sequence name (“POLYNTD SEQ NAME”) of the polynucleotide of SEQ ID NOS: 134-1352 that maps to the DNA and protein.

TABLE 16

| cDNA | | | | | |
|---|---|---|---|---|---|
| SEQ | | PROTEIN | PROTEIN SEQ | POLYNTD | POLYNTD SEQ |
| ID | cDNA SEQ NAME | SEQ ID | NAME | SEQ ID | NAME |
| 1562 | NTN_004511S11.3_4 | 1619 | NTP_004511S11.3_4 | 182 | 3544.B02.GZ43_505328 |
| 1563 | NTN_004754S1.3_4 | 1620 | NTP_004754S1.3_4 | 896 | 3608.E20.gz43_514326 |
| 1564 | NTN_005530S2.3_3 | 1621 | NTP_005530S2.3_3 | 662 | 3580.E23.GZ43_510293 |
| 1565 | NTN_005530S2.3_4 | 1622 | NTP_005530S2.3_4 | 662 | 3580.E23.GZ43_510293 |
| 1566 | NTN_005564S1.3_3 | 1623 | NTP_005564S1.3_3 | 383 | 3556.G15.GZ43_507077 |
| 1566 | NTN_005564S1.3_3 | 1623 | NTP_005564S1.3_3 | 283 | 3550.K05.GZ43_506153 |
| 1566 | NTN_005564S1.3_3 | 1623 | NTP_005564S1.3_3 | 519 | 3568.C22.GZ43_508739 |
| 1567 | NTN_005635S2.3_1 | 1624 | NTP_005635S2.3_1 | 551 | 3571.B22.GZ43_509122 |
| 1567 | NTN_005635S2.3_1 | 1624 | NTP_005635S2.3_1 | 550 | 3571.B13.GZ43_508978 |
| 1568 | NTN_005962S3.3_4 | 1625 | NTP_005962S3.3_4 | 386 | 3556.H12.GZ43_507030 |
| 1569 | NTN_006051S4.3_2 | 1626 | NTP_006051S4.3_2 | 448 | 3559.P10.GZ43_507390 |
| 1570 | NTN_006051S5.3_5 | 1627 | NTP_006051S5.3_5 | 448 | 3559.P10.GZ43_507390 |
| 1571 | NTN_007011S7.3_9 | 1628 | NTP_007011S7.3_9 | 983 | 3620.K24.gz43_516155 |
| 1571 | NTN_007011S7.3_9 | 1628 | NTP_007011S7.3_9 | 635 | 3577.J04.GZ43_509610 |
| 1572 | NTN_007122S8.3_8 | 1629 | NTP_007122S8.3_8 | 1337 | 3762.E15.gz43_534217 |
| 1572 | NTN_007122S8.3_8 | 1629 | NTP_007122S8.3_8 | 1337 | 3762.E15.gz43_534217 |
| 1573 | NTN_007592S2.3_10 | 1630 | NTP_007592S2.3_10 | 1005 | 3626.G01.gz43_516551 |
| 1574 | NTN_007592S3.3_5 | 1631 | NTP_007592S3.3_5 | 1057 | 3638.A02.gz43_518097 |
| 1575 | NTN_007844S7.3_3 | 1632 | NTP_007844S7.3_3 | 1038 | 3632.O06.gz43_517407 |
| 1576 | NTN_007867S7.3_3 | 1633 | NTP_007867S7.3_3 | 566 | 3571.H16.GZ43_509032 |
| 1577 | NTN_007867S8.3_1 | 1634 | NTP_007867S8.3_1 | 566 | 3571.H16.GZ43_509032 |
| 1578 | NTN_008059S2.3_3 | 1635 | NTP_008059S2.3_3 | 766 | 3590.H16.GZ43_512345 |
| 1579 | NTN_008338S4.3_6 | 1636 | NTP_008338S4.3_6 | 1014 | 3626.P14.gz43_516768 |
| 1579 | NTN_008338S4.3_6 | 1636 | NTP_008338S4.3_6 | 349 | 3553.L02.GZ43_506490 |
| 1580 | NTN_008470S7.3_1 | 1637 | NTP_008470S7.3_1 | 1233 | 3754.F15.gz43_533066 |
| 1581 | NTN_008627S7.3_5 | 1638 | NTP_008627S7.3_5 | 956 | 3617.C21.gz43_515492 |
| 1582 | NTN_008769S8.3_1 | 1639 | NTP_008769S8.3_1 | 207 | 3544.M10.GZ43_505467 |
| 1582 | NTN_008769S8.3_1 | 1639 | NTP_008769S8.3_1 | 207 | 3544.M10.GZ43_505467 |
| 1583 | NTN_008858S2.3_2 | 1640 | NTP_008858S2.3_2 | 844 | 3599.M04.GZ43_512926 |
| 1584 | NTN_009296S1.3_1 | 1641 | NTP_009296S1.3_1 | 955 | 3617.B16.gz43_515411 |
| 1585 | NTN_009296S3.3_2 | 1642 | NTP_009296S3.3_2 | 883 | 3605.I19.gz43_513930 |
| 1585 | NTN_009296S3.3_2 | 1642 | NTP_009296S3.3_2 | 1117 | 3661.K22.gz43_519717 |
| 1585 | NTN_009296S3.3_2 | 1642 | NTP_009296S3.3_2 | 968 | 3617.P11.gz43_515345 |
| 1586 | NTN_009526S2.3_3 | 1643 | NTP_009526S2.3_3 | 166 | 3541.I18.GZ43_505207 |
| 1586 | NTN_009526S2.3_3 | 1643 | NTP_009526S2.3_3 | 555 | 3571.E02.GZ43_508805 |
| 1586 | NTN_009526S2.3_3 | 1643 | NTP_009526S2.3_3 | 889 | 3605.N12.gz43_513823 |
| 1587 | NTN_009526S2.3_5 | 1644 | NTP_009526S2.3_5 | 555 | 3571.E02.GZ43_508805 |
| 1587 | NTN_009526S2.3_5 | 1644 | NTP_009526S2.3_5 | 166 | 3541.I18.GZ43_505207 |
| 1587 | NTN_009526S2.3_5 | 1644 | NTP_009526S2.3_5 | 889 | 3605.N12.gz43_513823 |
| 1588 | NTN_009848S8.3_5 | 1645 | NTP_009848S8.3_5 | 753 | 3590.D03.GZ43_512133 |
| 1589 | NTN_009866S24.3_5 | 1646 | NTP_009866S24.3_5 | 626 | 3577.E19.GZ43_509845 |
| 1590 | NTN_010018S2.3_5 | 1647 | NTP_010018S2.3_5 | 703 | 3580.P04.GZ43_510000 |
| 1591 | NTN_010164S3.3_8 | 1648 | NTP_010164S3.3_8 | 727 | 3583.K08.GZ43_510443 |
| 1592 | NTN_010289S6.3_5 | 1649 | NTP_010289S6.3_5 | 742 | 3583.O03.GZ43_510367 |
| 1593 | NTN_010663S1.3_1 | 1650 | NTP_010663S1.3_1 | 506 | 3565.N13.GZ43_508204 |
| 1594 | NTN_010757S4.3_2 | 1651 | NTP_010757S4.3_2 | 780 | 3590.M09.GZ43_512238 |
| 1595 | NTN_011059S6.3_4 | 1652 | NTP_011059S6.3_4 | 425 | 3559.E20.GZ43_507539 |
| 1596 | NTN_011130S2.3_3 | 1653 | NTP_011130S2.3_3 | 1226 | 3754.C22.gz43_533175 |
| 1596 | NTN_011130S2.3_3 | 1653 | NTP_011130S2.3_3 | 1226 | 3754.C22.gz43_533175 |
| 1597 | NTN_011266S2.2_3 | 1654 | NTP_011266S2.2_3 | 1209 | 3666.G12.gz43_521473 |
| 1598 | NTN_011361S6.3_7 | 1655 | NTP_011361S6.3_7 | 428 | 3559.H09.GZ43_507366 |
| 1599 | NTN_011430S6.3_6 | 1656 | NTP_011430S6.3_6 | 1195 | 3665.O19.gz43_521209 |
| 1600 | NTN_011512S51.3_3 | 1657 | NTP_011512S51.3_3 | 333 | 3553.K07.GZ43_506569 |
| 1601 | NTN_017582S2.3_6 | 1658 | NTP_017582S2.3_6 | 948 | 3614.L13.gz43_514989 |
| 1602 | NTN_019508S1.3_6 | 1659 | NTP_019508S1.3_6 | 956 | 3617.C21.gz43_515492 |
| 1603 | NTN_019721S6.3_1 | 1660 | NTP_019721S6.3_1 | 139 | 3541.A23.GZ43_505279 |
| 1604 | NTN_022448S1.3_2 | 1661 | NTP_022448S1.3_2 | 943 | 3614.G20.gz43_515096 |
| 1604 | NTN_022448S1.3_2 | 1661 | NTP_022448S1.3_2 | 915 | 3611.B16.gz43_514643 |
| 1604 | NTN_022448S1.3_2 | 1661 | NTP_022448S1.3_2 | 1087 | 3646.B20.gz43_519154 |
| 1605 | NTN_022456S1.3_2 | 1662 | NTP_022456S1.3_2 | 949 | 3614.M08.gz43_514910 |
| 1606 | NTN_022526S1.3_2 | 1663 | NTP_022526S1.3_2 | 1018 | 3629.E01.gz43_516933 |
| 1606 | NTN_022526S1.3_2 | 1663 | NTP_022526S1.3_2 | 1161 | 3664.A11.gz43_520683 |
| 1606 | NTN_022526S1.3_2 | 1663 | NTP_022526S1.3_2 | 1036 | 3632.N13.gz43_517518 |
| 1607 | NTN_022807S2.3_1 | 1664 | NTP_022807S2.3_1 | 1080 | 3643.O21.gz43_518799 |
| 1608 | NTN_022948S1.3_1 | 1665 | NTP_022948S1.3_1 | 1025 | 3629.J03.gz43_516970 |
| 1608 | NTN_022948S1.3_1 | 1665 | NTP_022948S1.3_1 | 1197 | 3665.O23.gz43_521273 |
| 1608 | NTN_022948S1.3_1 | 1665 | NTP_022948S1.3_1 | 1205 | 3666.D11.gz43_521454 |
| 1608 | NTN_022948S1.3_1 | 1665 | NTP_022948S1.3_1 | 965 | 3617.N10.gz43_515327 |
| 1608 | NTN_022948S1.3_1 | 1665 | NTP_022948S1.3_1 | 1105 | 3661.A08.gz43_519483 |
| 1608 | NTN_022948S1.3_1 | 1665 | NTP_022948S1.3_1 | 1177 | 3664.L21.gz43_520854 |
| 1608 | NTN_022948S1.3_1 | 1665 | NTP_022948S1.3_1 | 939 | 3614.D14.gz43_514997 |
| 1608 | NTN_022948S1.3_1 | 1665 | NTP_022948S1.3_1 | 1098 | 3646.K14.gz43_519067 |
| 1608 | NTN_022948S1.3_1 | 1665 | NTP_022948S1.3_1 | 938 | 3614.C18.gz43_515060 |
| 1609 | NTN_022948S1.3_5 | 1666 | NTP_022948S1.3_5 | 939 | 3614.D14.gz43_514997 |
| 1609 | NTN_022948S1.3_5 | 1666 | NTP_022948S1.3_5 | 1310 | 3759.L10.gz43_533760 |
| 1609 | NTN_022948S1.3_5 | 1666 | NTP_022948S1.3_5 | 965 | 3617.N10.gz43_515327 |
| 1610 | NTN_023142S2.3_4 | 1667 | NTP_023142S2.3_4 | 410 | 3556.O08.GZ43_506973 |
| 1611 | NTN_023860S1.3_1 | 1668 | NTP_023860S1.3_1 | 1180 | 3664.P18.gz43_520810 |
| 1612 | NTN_024001S1.3_4 | 1669 | NTP_024001S1.3_4 | 370 | 3556.B09.GZ43_506976 |
| 1612 | NTN_024001S1.3_4 | 1669 | NTP_024001S1.3_4 | 394 | 3556.K04.GZ43_506905 |
| 1613 | NTN_024871S1.3_7 | 1670 | NTP_024871S1.3_7 | 700 | 3580.O06.GZ43_510031 |
| 1614 | NTN_024882S1.3_3 | 1671 | NTP_024882S1.3_3 | 602 | 3574.G07.GZ43_509271 |
| 1615 | NTN_025842S13.2_1 | 1672 | NTP_025842S13.2_1 | 999 | 3623.N23.gz43_516526 |
| 1616 | NTN_025864S1.1_1 | 1673 | NTP_025864S1.1_1 | 1067 | 3643.F07.gz43_518566 |
| 1616 | NTN_025864S1.1_1 | 1673 | NTP_025864S1.1_1 | 1067 | 3643.F07.gz43_518566 |
| 1616 | NTN_025864S1.1_1 | 1673 | NTP_025864S1.1_1 | 1334 | 3762.D22.gz43_534328 |
| 1617 | NTN_025907S4.2_3 | 1674 | NTP_025907S4.2_3 | 1192 | 3665.O06.gz43_521001 |
| 1618 | NTN_026331S1.1_1 | 1675 | NTP_026331S1.1_1 | 566 | 3571.H16.GZ43_509032 |

Through contig and consensus sequence assembly and the use of homology searching software programs, the sequence information provided herein can be readily extended to confirm, or confirm a predicted, gene having the sequence of the polynucleotides described in the present invention. Further the information obtained can be used to identify the function of the gene product of the gene corresponding to the polynucleotides described herein. While not necessary to the practice of the invention, identification of the function of the corresponding gene, can provide guidance in the design of therapeutics that target the gene to modulate its activity and modulate the cancerous phenotype (e.g., inhibit metastasis, proliferation, and the like).

### Example 20

### Results of Public Database Search to Identify Function of Gene Products

SEQ ID NOS:134-1618 were translated in all three reading frames, and the nucleotide sequences and translated amino acid sequences used as query sequences to search for homologous sequences in the GenBank (nucleotide sequences) database. Query and individual sequences were aligned using the TeraBLAST program available from TimeLogic, Crystal Bay, Nev. The sequences were masked to various extents to prevent searching of repetitive sequences or poly-A sequences, using the RepeatMasker masking program for masking low complexity as described above.

Table 17 (inserted prior to claims) provides the alignment summaries having a p value of 1×10e-2 or less indicating substantial homology between the sequences of the present invention and those of the indicated public databases. Specifically, Table 17 provides: 1) the SEQ ID NO (“SEQ ID”) of the query sequence; 2) the sequence name (“SEQ NAME”) used as an internal identifier of the query sequence; 3) the accession number (“ACCESSION”) of the GenBank database entry of the homologous sequence; 4) a description of the GenBank sequences (“GENBANK DESCRIPTION”); and 5) the score of the similarity of the polynucleotide sequence and the GenBank sequence (“GENBANK SCORE”). The alignments provided in Table 8 are the best available alignment to a DNA sequence at a time just prior to filing of the present specification. Incorporated by reference is all publicly available information regarding the sequence listed in Table 17 and their related sequences. The search program and database used for the alignment, as well as the calculation of the p value are also indicated. Full length sequences or fragments of the polynucleotide sequences can be used as probes and primers to identify and isolate the full length sequence of the corresponding polynucleotide.

TABLE 17

| SEQ | | | | GENBANK |
|---|---|---|---|---|
| ID | SEQ NAME | ACCESSION | GENBANK DESCRIPTION | SCORE |
| 134 | 3538.O24.GZ43_504925 | AF047717 | Streptomyces chrysomallus actinomycin | 1.17E−04 |
| | | | synthetase II (acmB) gene, complete cds | |
| 135 | 3538.P11.GZ43_504718 | AF111848 | Homo sapiens PRO0529 mRNA, | 2.00E−06 |
| | | | complete cds | |
| 136 | 3541.A04.GZ43_504975 | X58178 | S. pyogenes for emm41 gene | 5.00E−06 |
| 137 | 3541.A05.GZ43_504991 | AF190638 | Mus musculus nephrin NPHS1 (Nphs1) | 2.00E−06 |
| | | | gene, partial cds | |
| 138 | 3541.A16.GZ43_505167 | AB024689 | Mus musculus gene, exon 3, partial | 6.00E−06 |
| | | | sequence | |
| 139 | 3541.A23.GZ43_505279 | M14155 | Human insulin-like growth factor (IGF-I) | 3.00E−06 |
| | | | IB gene, exon 4 | |
| 140 | 3541.B04.GZ43_504976 | U32801 | Haemophilus influenzae Rd section 116 | 1.10E−05 |
| | | | of 163 of the complete genome | |
| 141 | 3541.B17.GZ43_505184 | X89398 | H. sapiens ung gene for uracil DNA- | 1.21E−04 |
| | | | glycosylase | |
| 142 | 3538.G08.GZ43_504661 | AF270390 | Staphylococcus epidermidis strain SR1 | 3.00E−06 |
| | | | clone step.4045d08 genomic sequence | |
| 143 | 3538.G17.GZ43_504805 | AC006623 | Caenorhabditis elegans clone C52E2, | 4.00E−06 |
| | | | complete sequence | |
| 144 | 3538.G19.GZ43_504837 | AB042425 | Homo sapiens Pim-2h, hUGT2, hUGT1, | 6.60E−11 |
| | | | genes for pim-2 protooncogene homolog, | |
| | | | UDP-galactose transporter 1, UDP- | |
| | | | galactose transporter 2, complete cds | |
| 145 | 3538.G22.GZ43_504885 | L08338 | Human immunodeficiency virus type 1 | 3.10E−07 |
| | | | proviral envelope glycoprotein gene V3 | |
| | | | region from A196/4537, clone 6 (from | |
| | | | adult) | |
| 146 | 3538.H05.GZ43_504614 | AE006731 | Sulfolobus solfataricus section 90 of 272 | 2.00E−06 |
| | | | of the complete genome | |
| 147 | 3538.H21.GZ43_504870 | AL121807 | S. pombe chromosome III cosmid c132 | 1.30E−05 |
| 148 | 3538.I08.GZ43_504663 | AF186379 | Homo sapiens ligand effect modulator-6 | 8.00E−10 |
| | | | (LEM6) mRNA, complete cds | |
| 149 | 3538.I13.GZ43_504743 | AC007658 | Arabidopsis thaliana chromosome II | 3.30E−08 |
| | | | section 216 of 255 of the complete | |
| | | | sequence. Sequence from clones F27I1 | |
| 150 | 3538.J22.GZ43_504888 | X04616 | Anacystis nidulans R2 psbAI gene for | 8.90E−07 |
| | | | photosystem II Q(B) protein | |
| 151 | 3538.K12.GZ43_504729 | X91656 | M. musculus Srp20 gene | 4.40E−05 |
| 152 | 3538.K23.GZ43_504905 | M62849 | Human papillomavirus ORFs | 4.40E−07 |
| 153 | 3538.L16.GZ43_504794 | AE001382 | Plasmodium falciparum chromosome 2, | 7.00E−06 |
| | | | section 19 of 73 of the complete | |
| | | | sequence | |
| 154 | 3538.M02.GZ43_504571 | U07976 | Human T cell receptor beta | 7.00E−06 |
| | | | (TCRBV7S2, TCRBV13S2-1, | |
| | | | TCRBV6S7-1) genes, TCRBV deleted 2 | |
| | | | haplotype, partial cds | |
| 155 | 3538.M05.GZ43_504619 | AC079878 | Homo sapiens BAC clone RP11-343P21 | 1.40E−07 |
| | | | from 7, complete sequence | |
| 156 | 3538.M08.GZ43_504667 | AF182668 | Zenaida galapagoensis beta-fibrinogen | 4.70E−08 |
| | | | gene, partial sequence | |
| 157 | 3538.N20.GZ43_504860 | AB033411 | Taenia crassiceps mitochondrial gene for | 6.80E−07 |
| | | | cytochrome c oxidase subunit 1, partial | |
| | | | cds | |
| 158 | 3538.O07.GZ43_504653 | X68019 | Feline Immunodeficiency Virus GAG | 4.00E−06 |
| | | | gene | |
| 159 | 3541.E11.GZ43_505091 | M73447 | Human repeat polymorphism at locus | 3.00E−08 |
| | | | D9S59 | |
| 160 | 3541.E14.GZ43_505139 | AJ243419 | Acaulospora trappei partial 18S rRNA, | 1.10E−07 |
| | | | 5.8S rRNA and partial 28S rRNA genes | |
| | | | and internal transcribed spacers 1 and 2 | |
| | | | (ITS1, ITS2), isolate AU 219 | |
| 161 | 3541.E15.GZ43_505155 | U13679 | Human lactate dehydrogenase-A (LDH- | 3.50E−10 |
| | | | A) gene, promoter region | |
| 162 | 3541.G17.GZ43_505189 | AE004851 | Pseudomonas aeruginosa PA01, section | 1.30E−05 |
| | | | 412 of 529 of the complete genome | |
| 163 | 3541.H14.GZ43_505142 | AJ252202 | Drosophila melanogaster D-COQ7 gene | 9.00E−06 |
| | | | for putative COQ7 isologue, exons 1-3 | |
| 164 | 3541.I15.GZ43_505159 | X98371 | D. subobscura sex-lethal gene | 6.00E−06 |
| 165 | 3541.I17.GZ43_505191 | AK023918 | Homo sapiens cDNA FLJ13856 fis, | 1.70E−22 |
| | | | clone THYRO1000988 | |
| 166 | 3541.I18.GZ43_505207 | AF329081 | Bos taurus AMP-activated protein kinase | 5.30E−33 |
| | | | gamma-1 (PRKAG1) gene, partial cds | |
| 167 | 3541.J19.GZ43_505224 | AF002749 | Psychotria urceolata ribosomal protein | 3.01E−03 |
| | | | S16 (rps16) gene, chloroplast gene | |
| | | | encoding chloroplast protein, partial | |
| | | | intron | |
| 168 | 3541.K09.GZ43_505065 | AF027607 | Gallus gallus L-type voltage-gated | 9.00E−06 |
| | | | calcium channel alpha1D subunit | |
| | | | ChCaChA1D precursor mRNA, | |
| | | | complete intron sequence | |
| 169 | 3541.L19.GZ43_505226 | AE003949 | Xylella fastidiosa 9a5c, section 95 of 229 | 2.00E−06 |
| | | | of the complete genome | |
| 170 | 3541.M02.GZ43_504955 | BC004556 | Homo sapiens, Similar to CG7083 gene | 6.20E−07 |
| | | | product, clone MGC: 10534 | |
| | | | IMAGE: 3957147, mRNA, complete cds | |
| 171 | 3541.M07.GZ43_505035 | X05616 | Kangaroo rat repetitive DNA with | 4.80E−08 |
| | | | insertion sequence | |
| 172 | 3541.M18.GZ43_505211 | M81888 | Parvovirus LuII DNA sequence | 6.60E−05 |
| 173 | 3541.O04.GZ43_504989 | AF081828 | Ixodes hexagonus mitochondrial DNA, | 3.00E−06 |
| | | | complete genome | |
| 174 | 3541.O13.GZ43_505133 | AK026465 | Homo sapiens cDNA: FLJ22812 fis, | 8.00E−06 |
| | | | clone KAIA2955 | |
| 175 | 3541.O23.GZ43_505293 | X54859 | Porcine TNF-alpha and TNF-beta genes | 2.90E−05 |
| | | | for tumour necrosis factors alpha and | |
| | | | beta, respectively | |
| 176 | 3541.P05.GZ43_505006 | AE006642 | Sulfolobus solfataricus section 1 of 272 | 3.50E−05 |
| | | | of the complete genome | |
| 177 | 3541.P22.GZ43_505278 | U10400 | Saccharomyces cerevisiae chromosome | 1.80E−05 |
| | | | VIII cosmid L2825 | |
| 178 | 3544.A09.GZ43_505439 | X75677 | C. parapsilosis mt tRNA genes (591 bps) | 3.70E−08 |
| 179 | 3544.A13.GZ43_505503 | D28811 | Schistosoma japonicum mRNA for | 5.40E−05 |
| | | | paramyosin, complete cds | |
| 180 | 3544.A14.GZ43_505519 | M87111 | Human immunodeficiency virus type 2 | 2.90E−05 |
| | | | (FORTC2) reverse transcriptase | |
| | | | fragment | |
| 181 | 3544.A17.GZ43_505567 | L23650 | Caenorhabditis elegans cosmid C27D11, | 5.60E−07 |
| | | | complete sequence | |
| 182 | 3544.B02.GZ43_505328 | AF060543 | Homo sapiens importin alpha 7 subunit | 1.50E−49 |
| | | | mRNA, complete cds | |
| 183 | 3544.B09.GZ43_505440 | AB051473 | Homo sapiens mRNA for KIAA1686 | 1.80E−05 |
| | | | protein, partial cds | |
| 184 | 3544.B18.GZ43_505584 | AJ224821 | Loxodonta africana complete | 4.00E−06 |
| | | | mitochondrial genomic sequence | |
| 185 | 3544.E05.GZ43_505379 | AL451187 | Human DNA sequence from clone | 1.30E−07 |
| | | | RP11-49J23 on chromosome 6, complete | |
| | | | sequence [Homo sapiens] | |
| 186 | 3544.E18.GZ43_505587 | L08338 | Human immunodeficiency virus type 1 | 3.30E−07 |
| | | | proviral envelope glycoprotein gene V3 | |
| | | | region from A196/4537, clone 6 (from | |
| | | | adult) | |
| 187 | 3544.F06.GZ43_505396 | X60833 | R. norvegicus TDO2 gene for tryptophan | 7.80E−07 |
| | | | 2,3-dioxygenase, exon 6 | |
| 188 | 3544.F16.GZ43_505556 | U72716 | Drosophila melanogaster D3-100EF | 2.00E−06 |
| | | | mRNA, complete cds | |
| 189 | 3544.G06.GZ43_505397 | AC002359 | Homo sapiens Xp22 Cosmid U239B3 | 1.60E−05 |
| | | | (from Lawrence Livermore X library) | |
| | | | complete sequence | |
| 190 | 3544.G10.GZ43_505461 | X56015 | Crithidia oncopelti mitochondrial ND4, | 4.80E−05 |
| | | | ND5, COI, 12S ribosomal RNA genes | |
| | | | for NADH dehydrogenase subunit 4/5, | |
| | | | cytochrome oxidase subunit I and 12S | |
| | | | ribosomal RNA | |
| 191 | 3544.G11.GZ43_505477 | U80927 | Dictyostelium discoideum unknown | 9.00E−08 |
| | | | protein gene, complete cds | |
| 192 | 3544.G12.GZ43_505493 | AF245483 | Oryza sativa OSE4 (OSE4) gene, | 1.70E−07 |
| | | | complete cds | |
| 193 | 3544.H03.GZ43_505350 | Y12855 | Homo sapiens P2X7 gene, exon 12 and | 2.30E−05 |
| | | | 13 | |
| 194 | 3544.H15.GZ43_505542 | AF194829 | Tetragonia tetragonioides NADH | 2.00E−06 |
| | | | dehydrogenase (ndhF) gene, partial cds; | |
| | | | chloroplast gene for chloroplast product | |
| 195 | 3544.H24.GZ43_505686 | BC008353 | Homo sapiens, Similar to RIKEN cDNA | 2.50E−18 |
| | | | 0610008P16 gene, clone MGC: 15937 | |
| | | | IMAGE: 3537224, mRNA, complete cds | |
| 196 | 3544.I07.GZ43_505415 | AF010533 | Plasmodium falciparum microsatellite | 1.80E−08 |
| | | | TA21 sequence | |
| 197 | 3544.I15.GZ43_505543 | D29794 | Mouse gene for T cell receptor gamma | 3.00E−06 |
| | | | chain | |
| 198 | 3544.I20.GZ43_505623 | AE000677 | Aquifex aeolicus section 9 of 109 of the | 4.00E−06 |
| | | | complete genome | |
| 199 | 3544.J04.GZ43_505368 | Z97015 | Lactococcus lactis cremoris sucrose gene | 1.00E−06 |
| | | | cluster | |
| 200 | 3544.J11.GZ43_505480 | M67480 | Human prothymosin-alpha gene, | 5.10E−10 |
| | | | complete cds | |
| 201 | 3544.J13.GZ43_505512 | AJ249884 | Lepeophtheirus salmonis microsatellite | 5.70E−08 |
| | | | DNA, locus Ls.NUIG.09 | |
| 202 | 3544.J23.GZ43_505672 | AJ245823 | Trypanosoma brucei PK4 gene for | 6.00E−06 |
| | | | protein kinase | |
| 203 | 3544.K16.GZ43_505561 | U18191 | Human HLA class I genomic survey | 2.50E−07 |
| | | | sequence | |
| 204 | 3544.L11.GZ43_505482 | X07127 | Kluyveromyces lactis killer plasmid k1 | 2.90E−05 |
| | | | DNA | |
| 205 | 3544.L13.GZ43_505514 | BC005028 | Homo sapiens, hypothetical protein | 1.80E−31 |
| | | | FLJ11323, clone MGC: 12582 | |
| | | | IMAGE: 3953383, mRNA, complete cds | |
| 206 | 3544.M06.GZ43_505403 | AC006687 | Caenorhabditis elegans cosmid T20C7, | 2.30E−05 |
| | | | complete sequence | |
| 207 | 3544.M10.GZ43_505467 | M92378 | Mus musculus GABA transporter | 1.30E−05 |
| | | | mRNA sequence | |
| 208 | 3544.N07.GZ43_505420 | U48705 | Human receptor tyrosine kinase DDR | 7.40E−07 |
| | | | gene, complete cds | |
| 209 | 3544.N12.GZ43_505500 | BC007621 | Homo sapiens, Similar to Orthodenticle | 5.70E−07 |
| | | | (Drosophila) homolog 1, clone | |
| | | | MGC: 15736 IMAGE: 3355563, mRNA, | |
| | | | complete cds | |
| 210 | 3544.N19.GZ43_505612 | AF270077 | Staphylococcus epidermidis strain SR1 | 2.00E−07 |
| | | | clone step.1047c06 genomic sequence | |
| 211 | 3544.O03.GZ43_505357 | U15681 | Myrmecia pilosula HI87-156 | 1.00E−06 |
| | | | mitochondrion cytochrome b gene, | |
| | | | partial cds | |
| 212 | 3544.O10.GZ43_505469 | AF056032 | Homo sapiens kynurenine 3-hydroxylase | 5.00E−06 |
| | | | mRNA, complete cds | |
| 213 | 3544.O15.GZ43_505549 | U37373 | Xenopus laevis tail-specific thyroid | 3.00E−06 |
| | | | hormone up-regulated (gene 5) mRNA, | |
| | | | complete cds | |
| 214 | 3544.O20.GZ43_505629 | D66906 | Bombyx mori DNA for sorbitol | 2.00E−06 |
| | | | dehydrogenase, complete cds | |
| 215 | 3544.P18.GZ43_505598 | J04357 | Red clover necrotic mosaic virus RNA-1, | 4.00E−06 |
| | | | complete sequence | |
| 216 | 3547.A04.GZ43_505743 | AF118558 | Mus musculus hitchhiker-3, hitchhiker-4, | 5.40E−07 |
| | | | and hitchhiker-5 mRNA sequences | |
| 217 | 3547.A11.GZ43_505855 | U93874 | Bacillus subtilis cysteine synthase | 4.00E−06 |
| | | | (yrhA), cystathionine gamma-lyase | |
| | | | (yrhB), YrhC (yrhC), YrhD (yrhD), | |
| | | | formate dehydrogenase chain A (yrhE), | |
| | | | YrhF (yrhF), formate dehydrogenase | |
| | | | (yrhG), YrhH (yrhH), regulatory protein | |
| | | | (yrhI), cytochrome P450 102 (yrhJ),> | |
| 218 | 3547.A24.GZ43_506063 | AL157466 | Homo sapiens mRNA; cDNA | 8.80E−07 |
| | | | DKFZp761E2423 (from clone | |
| | | | DKFZp761E2423) | |
| 219 | 3547.C05.GZ43_505761 | X52589 | Bovine rotavirus RNA for virus protein 2 | 1.00E−05 |
| | | | (VP2) | |
| 220 | 3547.C17.GZ43_505953 | U67594 | Methanococcus jannaschii section 136 of | 3.80E−05 |
| | | | 150 of the complete genome | |
| 221 | 3547.C23.GZ43_506049 | AJ250862 | Bacillus sp. HIL-Y85/54728 mersacidin | 1.20E−05 |
| | | | biosynthesis gene cluster (mrsK2, | |
| | | | mrsR2, mrsF, mrsG, mrsE, mrsA, | |
| | | | mrsR1, mrsD, mrsM and mrsT genes) | |
| 222 | 3547.D19.GZ43_505986 | AF050491 | Microgadus tomcod aromatic | 4.00E−06 |
| | | | hydrocarbon receptor (ahr) gene, exons | |
| | | | 8-11, partial cds | |
| 223 | 3547.D23.GZ43_506050 | M33190 | Rat cytochrome P450 II A3 (CYP2A3) | 5.80E−05 |
| | | | gene, complete cds | |
| 224 | 3547.E04.GZ43_505747 | L35658 | Homo sapiens (subclone H8 9_d12 from | 7.70E−07 |
| | | | P1 35 H5 C8) DNA sequence | |
| 225 | 3547.F02.GZ43_505716 | AF038190 | Homo sapiens clone 23582 mRNA | 1.10E−07 |
| | | | sequence | |
| 226 | 3547.F10.GZ43_505844 | AY008833 | Staphylococcus aureus tcaR-tcaA-tcaB | 5.00E−06 |
| | | | operon, complete sequences | |
| 227 | 3547.F20.GZ43_506004 | AB037821 | Homo sapiens mRNA for KIAA1400 | 1.00E−06 |
| | | | protein, partial cds | |
| 228 | 3547.G02.GZ43_505717 | M88397 | Naegleria fowleri virulence-related | 3.70E−07 |
| | | | protein (NF314) mRNA, complete cds | |
| 229 | 3547.G09.GZ43_505829 | AJ315644 | Homo sapiens mRNA for proton myoinositol | 7.90E−07 |
| | | | symporter (Hmit gene) | |
| 230 | 3547.G22.GZ43_506037 | Z33603 | P. radiata (Pr1.6) microsatellite DNA, | 1.70E−07 |
| | | | 703 bp | |
| 231 | 3547.H12.GZ43_505878 | L04309 | Shigella flexneri ipgD, ipgE, ipgF genes, | 3.00E−06 |
| | | | complete cds | |
| 232 | 3547.H14.GZ43_505910 | AL137502 | Homo sapiens mRNA; cDNA | 2.90E−07 |
| | | | DKFZp761H171 (from clone | |
| | | | DKFZp761H171); partial cds | |
| 233 | 3547.I07.GZ43_505799 | M15332 | B. sphaericus ermG gene encoding rRNA | 7.00E−06 |
| | | | methyltransferase (macrolide- | |
| | | | lincosamide-streptogramin B resistance | |
| | | | element) | |
| 234 | 3547.I16.GZ43_505943 | AF015157 | Homo sapiens clone HS19.12 Alu-Ya5 | 4.70E−10 |
| | | | sequence | |
| 235 | 3547.I17.GZ43_505959 | AE007758 | Clostridium acetobutylicum ATCC824 | 3.00E−06 |
| | | | section 246 of 356 of the complete | |
| | | | genome | |
| 236 | 3547.I20.GZ43_506007 | L37606 | Medicago sativa (clone GG16-1) | 1.50E−05 |
| | | | NADH-dependent glutamate synthase | |
| | | | gene, complete cds | |
| 237 | 3547.J05.GZ43_505768 | Z16911 | H. sapiens (D20S113) DNA segment | 2.80E−07 |
| | | | containing (CA) repeat; clone | |
| | | | AFM205th8; single read | |
| 238 | 3547.J10.GZ43_505848 | Z37803 | HIV-1 DNA V3 region (patient 15, | 8.80E−07 |
| | | | sample CSF, clone 9) | |
| 239 | 3547.J20.GZ43_506008 | AF013273 | Candida albicans histidine kinase 1 gene, | 3.30E−05 |
| | | | complete cds | |
| 240 | 3547.J22.GZ43_506040 | AF289080 | Lycopersicon esculentum alpha- | 4.00E−06 |
| | | | galactosidase gene, partial cds | |
| 241 | 3547.K01.GZ43_505705 | AF267863 | Homo sapiens DC43 mRNA, complete | 7.30E−22 |
| | | | cds | |
| 242 | 3547.L09.GZ43_505834 | Z22175 | Caenorhabditis elegans cosmid K01F9, | 1.40E−05 |
| | | | complete sequence | |
| 243 | 3547.L11.GZ43_505866 | AJ288648 | Limnodynastes tasmaniensis | 5.90E−07 |
| | | | mitochondrial partial nadh4 gene for | |
| | | | NADH dehydrogenase subunit 4 and | |
| | | | partial tRNA-His gene, sample 26 from | |
| | | | Australia: Boolara | |
| 244 | 3547.L16.GZ43_505946 | AE001293 | Chlamydia trachomatis section 20 of 87 | 7.10E−07 |
| | | | of the complete genome | |
| 245 | 3547.L22.GZ43_506042 | AF287006 | Danio rerio T-box brain 1 mRNA, partial | 7.00E−06 |
| | | | cds | |
| 246 | 3547.M02.GZ43_505723 | AE007788 | Clostridium acetobutylicum ATCC824 | 1.00E−05 |
| | | | section 276 of 356 of the complete | |
| | | | genome | |
| 247 | 3547.M07.GZ43_505803 | Z46252 | M. musculus DNA for region | 6.00E−06 |
| | | | surrounding retrovirus restriction locus | |
| | | | Fv1 | |
| 248 | 3547.M08.GZ43_505819 | AB020684 | Homo sapiens mRNA for KIAA0877 | 1.50E−05 |
| | | | protein, partial cds | |
| 249 | 3547.M16.GZ43_505947 | AF335240 | Petunia x hybrida MADS-box | 3.00E−06 |
| | | | transcription factor FBP22 (FBP22) | |
| | | | mRNA, complete cds | |
| 250 | 3547.N06.GZ43_505788 | AF299346 | Renispora flavissima isolate CEH313 | 1.70E−08 |
| | | | 18S ribosomal RNA gene, partial | |
| | | | sequence; internal transcribed spacer 1, | |
| | | | 5.8S ribosomal RNA gene and internal | |
| | | | transcribed spacer 2, complete sequence; | |
| | | | and 28S ribosomal RNA gene, partial | |
| | | | sequence | |
| 251 | 3547.O03.GZ43_505741 | AE002344 | Chlamydia muridarum, section 72 of 85 | 6.60E−07 |
| | | | of the complete genome | |
| 252 | 3547.O07.GZ43_505805 | D50608 | Rat gene for cholecystokinin type-A | 1.60E−05 |
| | | | receptor (CCKAR), complete cds | |
| 253 | 3547.O14.GZ43_505917 | AL137502 | Homo sapiens mRNA; cDNA | 2.90E−07 |
| | | | DKFZp761H171 (from clone | |
| | | | DKFZp761H171); partial cds | |
| 254 | 3547.P18.GZ43_505982 | AJ131734 | Plasmodium berghei DNA including | 6.10E−07 |
| | | | upstream sequence NTS and 5′ETS of | |
| | | | the 18S rRNA gene (A rRNA gene unit) | |
| 255 | 3547.P21.GZ43_506030 | AC006619 | Caenorhabditis elegans cosmid C46C11, | 1.70E−05 |
| | | | complete sequence | |
| 256 | 3547.P22.GZ43_506046 | AJ000871 | Streptococcus mitis comC, comD, comE | 2.00E−06 |
| | | | genes, isolate B5 | |
| 257 | 3550.A12.GZ43_506255 | M22310 | Human epidermal growth factor receptor | 4.80E−07 |
| | | | proto-oncogene downstream enhancer | |
| 258 | 3550.A16.GZ43_506319 | L39435 | Senecio mikanioides chloroplast NADH | 2.00E−06 |
| | | | dehydrogenase (ndhF) gene, complete | |
| | | | cds | |
| 259 | 3550.B06.GZ43_506160 | D14161 | Hordeum vulgare ids-4 mRNA, complete | 1.10E−08 |
| | | | cds | |
| 260 | 3550.C01.GZ43_506081 | AK025182 | Homo sapiens cDNA: FLJ21529 fis, | 4.20E−09 |
| | | | clone COL05981 | |
| 261 | 3550.C22.GZ43_506417 | X52028 | Rattus norvegicus P450 IID3 gene | 1.41E−04 |
| 262 | 3550.D16.GZ43_506322 | Y10345 | H. sapiens GalNAc-T3 gene, 3′UTR | 5.00E−07 |
| 263 | 3550.D23.GZ43_506434 | AF134403 | Escherichia coli plasmid pAA2 Shf (shf), | 6.90E−07 |
| | | | hexosyltransferase homolog (capU), and | |
| | | | VirK (virK) genes, complete cds | |
| 264 | 3550.E02.GZ43_506099 | U66074 | Tritrichomonas foetus putative | 8.90E−07 |
| | | | superoxide dismutase 2 (SOD2) gene, | |
| | | | complete cds | |
| 265 | 3550.E06.GZ43_506163 | Z23341 | H. sapiens (D8S528) DNA segment | 2.30E−08 |
| | | | containing (CA) repeat; clone | |
| | | | AFM080xh7; single read | |
| 266 | 3550.F06.GZ43_506164 | M59447 | Drosophila melanogaster Sex-lethal | 3.00E−06 |
| | | | (Sx1) mRNA, complete cds | |
| 267 | 3550.F08.GZ43_506196 | M24901 | Rabbit pulmonary surfactant-associated | 3.40E−07 |
| | | | protein (SP-B) mRNA, complete cds | |
| 268 | 3550.F20.GZ43_506388 | AF216169 | Simicratea welwitschii clone 2 | 5.40E−08 |
| | | | phytochrome B (PHYB) gene, exon 1 | |
| | | | and partial cds | |
| 269 | 3550.F22.GZ43_506420 | AP000739 | Arabidopsis thaliana genomic DNA, | 2.20E−05 |
| | | | chromosome 3, P1 clone: MEK6 | |
| 270 | 3550.G02.GZ43_506101 | AL022342 | Human DNA sequence from clone RP1- | 7.40E−05 |
| | | | 29M10 on chromosome 20, complete | |
| | | | sequence [Homo sapiens] | |
| 271 | 3550.G08.GZ43_506197 | AK021312 | Mus musculus 13 days embryo stomach | 3.60E−08 |
| | | | cDNA, RIKEN full-length enriched | |
| | | | library, clone: D530039A21, full insert | |
| | | | sequence | |
| 272 | 3550.G10.GZ43_506229 | M31684 | D. melanogaster cytoskeleton-like | 3.00E−06 |
| | | | bicaudalD protein (BicD) mRNA, | |
| | | | complete cds | |
| 273 | 3550.G15.GZ43_506309 | AF087141 | Mus musculus uncharacterized long | 4.00E−06 |
| | | | terminal repeat, complete sequence; and | |
| | | | valyl-tRNA synthetase (G7a) gene, | |
| | | | complete cds | |
| 274 | 3550.G23.GZ43_506437 | X02547 | Trypanosoma brucei mitochondrial | 2.00E−06 |
| | | | genes for 12S and 9S ribosomal RNA | |
| 275 | 3550.H10.GZ43_506230 | U55711 | Human ataxia-telangiectasia (ATM) | 6.10E−08 |
| | | | gene, exon 11 | |
| 276 | 3550.H21.GZ43_506406 | Z68755 | Human DNA sequence from cosmid | 2.00E−06 |
| | | | L118D5, Huntington's Disease Region, | |
| | | | chromosome 4p16.3 | |
| 277 | 3550.H23.GZ43_506438 | AF151388 | Dermatobia hominis strain Alfenas | 1.20E−07 |
| | | | tRNA-Ile gene, partial sequence; D-loop, | |
| | | | complete sequence; and 12S ribosomal | |
| | | | RNA, partial sequence; mitochondrial | |
| | | | genes for mitochondrial products | |
| 278 | 3550.I03.GZ43_506119 | AF117258 | Staphylococcus aureus plasmid pIP680 | 6.50E−08 |
| | | | replication protein RepE (repE) gene, | |
| | | | partial cds; resolvase (res), | |
| | | | acetyltransferase Vat (vat), and | |
| | | | hydrolase VgB (vgb) genes, complete | |
| | | | cds; and unknown gene | |
| 279 | 3550.I19.GZ43_506375 | AE002781 | Drosophila melanogaster genomic | 3.90E−05 |
| | | | scaffold 142000013385442, complete | |
| | | | sequence | |
| 280 | 3550.I21.GZ43_506407 | AE001002 | Archaeoglobus fulgidus section 105 of | 4.20E−05 |
| | | | 172 of the complete genome | |
| 281 | 3550.J05.GZ43_506152 | AF080689 | Homo sapiens protein kinase PITSLRE | 5.50E−10 |
| | | | (CDC2L2) gene, exons 8 and 9 | |
| 282 | 3550.J11.GZ43_506248 | Z82761 | R. prowazekii genomic DNA fragment | 1.00E−06 |
| | | | (clone A793R) | |
| 283 | 3550.K05.GZ43_506153 | X15407 | Maize pseudo-Gpa2 pseudogene for | 3.20E−05 |
| | | | glyceraldehyde-3-phosphate | |
| | | | dehydrogenase subunit A | |
| 284 | 3550.K09.GZ43_506217 | X62631 | S. pombe wis1 gene for protein kinase | 1.50E−07 |
| 285 | 3550.K14.GZ43_506297 | M59743 | Rabbit cardiac muscle Ca-2+ release | 1.00E−06 |
| | | | channel (ryanodine receptor) mRNA, | |
| | | | complete cds | |
| 286 | 3550.L16.GZ43_506330 | AF201383 | Buchnera aphidicola isopropylmalate | 1.00E−06 |
| | | | dehydratase subunit (leuC) gene, partial | |
| | | | cds | |
| 287 | 3550.L19.GZ43_506378 | M77244 | H. sapiens erythropoietin receptor | 4.00E−09 |
| | | | (EPOR) gene, 5′ end | |
| 288 | 3550.L23.GZ43_506442 | L76259 | Homo sapiens PTS gene, complete cds | 8.00E−06 |
| 289 | 3550.M21.GZ43_506411 | M87339 | Human replication factor C, 37-kDa | 5.00E−06 |
| | | | subunit mRNA, complete cds | |
| 290 | 3550.N01.GZ43_506092 | AF191009 | Helicobacter pylori strain ChinaF30A | 1.10E−07 |
| | | | cag pathogenicity island polymorphic | |
| | | | right end, type IIIa motif | |
| 291 | 3550.N07.GZ43_506188 | AF235499 | Mus musculus SH2-containing inositol | 1.55E−04 |
| | | | 5-phosphatase (Ship) gene, exons 3 | |
| | | | through 6 | |
| 292 | 3550.O03.GZ43_506125 | D14813 | Human DNA for osteopontin, complete | 4.50E−05 |
| | | | cds | |
| 293 | 3550.O04.GZ43_506141 | U08596 | Canis familiaris delayed rectifier K+ | 6.00E−06 |
| | | | channel mRNA, partial cds | |
| 294 | 3550.O08.GZ43_506205 | XM_017044 | Homo sapiens similar to diaphanous | 6.40E−09 |
| | | | (Drosophila, homolog) 2 (H. sapiens) | |
| | | | (LOC91459), mRNA | |
| 295 | 3550.O15.GZ43_506317 | U15977 | Mus musculus long chain fatty acyl CoA | 2.80E−05 |
| | | | synthetase mRNA, complete cds | |
| 296 | 3550.O17.GZ43_506349 | X62578 | C. caldarium plastid genes ompR′, psbD, | 2.80E−05 |
| | | | psbC, rps16 and groEL | |
| 297 | 3550.O18.GZ43_506365 | L34363 | Human X-linked nuclear protein (XNP) | 4.00E−06 |
| | | | gene, complete cds | |
| 298 | 3550.O21.GZ43_506413 | AB056784 | Macaca fascicularis brain cDNA | 5.20E−07 |
| | | | clone: QnpA-11501, full insert sequence | |
| 299 | 3550.P18.GZ43_506366 | AK002041 | Homo sapiens cDNA FLJ11179 fis, | 5.30E−07 |
| | | | clone PLACE1007450 | |
| 300 | 3550.P23.GZ43_506446 | AF200361 | Rattus norvegicus cytochrome P450 4F1 | 1.40E−05 |
| | | | (Cyp4F1) gene, complete cds | |
| 301 | 3553.A09.GZ43_506591 | AL109980 | Human DNA sequence from clone RP4- | 3.50E−12 |
| | | | 697G8 on chromosome 22, complete | |
| | | | sequence [Homo sapiens] | |
| 302 | 3553.B07.GZ43_506560 | L37056 | Strongylocentrotus purpuratus myc | 4.60E−07 |
| | | | protein mRNA, complete cds | |
| 303 | 3553.B16.GZ43_506704 | U43542 | Nicotiana tabacum diphenol oxidase | 2.00E−06 |
| | | | mRNA, complete cds | |
| 304 | 3553.B22.GZ43_506800 | L34040 | Homo sapiens stromelysin gene, | 6.00E−06 |
| | | | promoter region | |
| 305 | 3553.D04.GZ43_506514 | Y07599 | S. pombe mRNA for dmf1 gene | 9.40E−07 |
| 306 | 3553.D07.GZ43_506562 | X13835 | R. norvegicus CaMII gene, exons 3, 4 & 5 | 2.00E−06 |
| 307 | 3553.D14.GZ43_506674 | L38424 | Bacillus subtilis dihydropicolinate | 1.80E−05 |
| | | | reductase (jojE) gene, complete cds; | |
| | | | poly(A) polymerase (jojI) gene, | |
| | | | complete cds; biotin acetyl-CoA- | |
| | | | carboxylase ligase (birA) gene, complete | |
| | | | cds; jojC, jojD, jojF, jojG, jojH genes, | |
| | | | complete cds's | |
| 308 | 3553.D19.GZ43_506754 | X53431 | Yeast gene for STE11 | 9.00E−06 |
| 309 | 3553.E08.GZ43_506579 | AF062863 | Arabidopsis thaliana putative | 1.80E−07 |
| | | | transcription factor (MYB11) mRNA, | |
| | | | partial cds | |
| 310 | 3553.E09.GZ43_506595 | X71067 | X. laevis XFG 5-1 and XFG 5-2 genes for | 6.60E−05 |
| | | | zinc finger proteins | |
| 311 | 3553.F12.GZ43_506644 | X63223 | B. taurus CI-MNLL mRNA for | 6.90E−08 |
| | | | ubiquinone oxidoreductase complex | |
| 312 | 3553.F13.GZ43_506660 | L81869 | Homo sapiens (subclone 1_c4 from P1 | 3.00E−08 |
| | | | H55) DNA sequence, complete sequence | |
| 313 | 3553.F19.GZ43_506756 | U97190 | Caenorhabditis elegans cosmid B0025, | 3.00E−06 |
| | | | complete sequence | |
| 314 | 3553.G05.GZ43_506533 | S76404 | beta-HKA = H, K-ATPase beta-subunit | 8.00E−06 |
| | | | [rats, Genomic, 8983 nt, segment 2 of 2] | |
| 315 | 3553.G06.GZ43_506549 | X68048 | Phaseolus vulgaris chloroplast DNA for | 5.00E−06 |
| | | | tRNA-His gene region | |
| 316 | 3553.G07.GZ43_506565 | AF068289 | Homo sapiens HDCMD34P mRNA, | 4.40E−12 |
| | | | complete cds | |
| 317 | 3553.G21.GZ43_506789 | Z33603 | P. radiata (Pr1.6) microsatellite DNA, | 1.70E−07 |
| | | | 703 bp | |
| 318 | 3553.H06.GZ43_506550 | AF090901 | Homo sapiens clone HQ0195$ PRO0195 | 8.00E−07 |
| | | | mRNA, complete cds | |
| 319 | 3553.H09.GZ43_506598 | AF270105 | Staphylococcus epidermidis strain SR1 | 9.80E−07 |
| | | | clone step.1049c09 genomic sequence | |
| 320 | 3553.H21.GZ43_506790 | Z18359 | Glycine max seed-specific low molecular | 2.00E−06 |
| | | | weight sulfur-rich protein | |
| 321 | 3553.I13.GZ43_506663 | AF155115 | Homo sapiens NY-REN-58 antigen | 1.70E−07 |
| | | | mRNA, complete cds | |
| 322 | 3553.I16.GZ43_506711 | AF270229 | Staphylococcus epidermidis strain SR1 | 1.20E−05 |
| | | | clone step.1055d10 genomic sequence | |
| 323 | 3553.J12.GZ43_506648 | U53400 | Rattus norvegicus chromosome 10 | 1.55E−01 |
| | | | microsatellite sequence D10Mco21 | |
| 324 | 3553.J14.GZ43_506680 | M10014 | Homo sapiens map 4q28 fibrinogen | 9.00E−06 |
| | | | (FGG) gene, alternative splice products, | |
| | | | complete cds | |
| 325 | 3553.J16.GZ43_506712 | Z23341 | H. sapiens (D8S528) DNA segment | 2.30E−08 |
| | | | containing (CA) repeat; clone | |
| | | | AFM080xh7; single read | |
| 326 | 3553.J17.GZ43_506728 | AK021312 | Mus musculus 13 days embryo stomach | 3.60E−08 |
| | | | cDNA, RIKEN full-length enriched | |
| | | | library, clone: D530039A21, full insert | |
| | | | sequence | |
| 327 | 3553.J22.GZ43_506808 | AF120279 | Mus musculus proline dehydrogenase | 5.00E−06 |
| | | | mRNA, complete cds | |
| 328 | 3553.J24.GZ43_506840 | Z18359 | Glycine max seed-specific low molecular | 2.00E−06 |
| | | | weight sulfur-rich protein | |
| 329 | 3553.K01.GZ43_506473 | U31465 | Kluyveromyces lactis telomerase RNA | 2.00E−06 |
| | | | component (TER1) gene, complete | |
| | | | sequence | |
| 330 | 3553.K02.GZ43_506489 | X60672 | M. musculus mRNA for radixin | 1.00E−06 |
| 331 | 3553.K03.GZ43_506505 | Z71943 | G. hyalina (92-89) DNA for internal | 1.06E−02 |
| | | | transcribed spacer 1 | |
| 332 | 3553.K05.GZ43_506537 | M80596 | Saccharomyces cerevisiae VAC1 gene | 6.00E−06 |
| | | | (required for vacuole inheritance and | |
| | | | vacuole protein sorting), complete cds | |
| 333 | 3553.K07.GZ43_506569 | AJ275317 | Cicer arietinum partial mRNA for malate | 7.60E−07 |
| | | | dehydrogenase | |
| 334 | 3553.K15.GZ43_506697 | X57377 | Mouse dilute myosin heavy chain gene | 2.40E−05 |
| | | | for novel heavy chain with unique C- | |
| | | | terminal region | |
| 335 | 3538.A11.GZ43_504703 | Z75199 | S. cerevisiae chromosome XV reading | 6.00E−06 |
| | | | frame ORF YOR291w | |
| 336 | 3538.A24.GZ43_504911 | AF270077 | Staphylococcus epidermidis strain SR1 | 2.00E−07 |
| | | | clone step.1047c06 genomic sequence | |
| 337 | 3538.B01.GZ43_504544 | AF368255 | Arabidopsis thaliana small zinc finger- | 4.10E−07 |
| | | | like protein TIM13 mRNA, complete | |
| | | | cds; nuclear gene for mitochondrial | |
| | | | product | |
| 338 | 3538.B20.GZ43_504848 | AB069994 | Macaca fascicularis testis cDNA | 1.40E−07 |
| | | | clone: QtsA-10636, full insert sequence | |
| 339 | 3538.C01.GZ43_504545 | AF072375 | Pseudoalteromonas sp. S9 beta- | 1.50E−04 |
| | | | hexosaminidase (chiP) gene, complete | |
| | | | cds | |
| 340 | 3538.C02.GZ43_504561 | AJ011271 | Human immunodeficiency virus type 2 | 7.10E−08 |
| | | | partial env gene, isolate b1286 | |
| 341 | 3538.D06.GZ43_504626 | AF100765 | Oryza sativa receptor-like kinase | 3.00E−06 |
| | | | (8ARK1) gene, complete cds | |
| 342 | 3538.D09.GZ43_504674 | Z47784 | M. musculus mRNA expressed in islet | 3.40E−08 |
| | | | cells (clone 58) | |
| 343 | 3538.D21.GZ43_504866 | AE006568 | Streptococcus pyogenes M1 GAS strain | 6.00E−07 |
| | | | SF370, section 97 of 167 of the complete | |
| | | | genome | |
| 344 | 3538.E15.GZ43_504771 | AB027966 | Schizosaccharomyces pombe gene for | 2.30E−08 |
| | | | Hypothetical protein, partial cds, | |
| | | | clone: TB89 | |
| 345 | 3538.F02.GZ43_504564 | AK001871 | Homo sapiens cDNA FLJ11009 fis, | 5.90E−09 |
| | | | clone PLACE1003108 | |
| 346 | 3538.F08.GZ43_504660 | U39161 | Human phosphodiesterase (PDEA) gene, | 7.40E−07 |
| | | | intron 8, 5′ end | |
| 347 | 3553.K23.GZ43_506825 | Y12052 | Homo sapiens gene encoding guanine | 3.00E−06 |
| | | | nucleotide-binding protein beta3 subunit, | |
| | | | exon 5 | |
| 348 | 3553.K24.GZ43_506841 | AP001419 | Homo sapiens genomic DNA, | 1.00E−06 |
| | | | chromosome 21q22.2, clone: PAC24K9, | |
| | | | LB7T-ERG region, complete sequence | |
| 349 | 3553.L02.GZ43_506490 | X15028 | Chicken hsp90 gene for 90 kDa-heat | 3.60E−05 |
| | | | shock protein 5′-end | |
| 350 | 3553.L04.GZ43_506522 | L34665 | Rattus norvegicus H+/K+-ATPase beta | 1.20E−09 |
| | | | subunit (HKB) gene, exon 6 | |
| 351 | 3553.L21.GZ43_506794 | M87843 | Human transforming growth factor beta- | 2.30E−05 |
| | | | 2 gene, 5′ end | |
| 352 | 3553.M12.GZ43_506651 | AB026592 | Limnoporus esakii mitochondrial gene | 1.10E−07 |
| | | | for 16S ribosomal RNA, partial sequence | |
| 353 | 3553.M23.GZ43_506827 | AE006349 | Lactococcus lactis subsp. lactis IL1403 | 8.00E−07 |
| | | | section 111 of 218 of the complete | |
| | | | genome | |
| 354 | 3553.N01.GZ43_506476 | U80457 | Human transcription factor SIM2 short | 2.00E−06 |
| | | | form mRNA, complete cds | |
| 355 | 3553.N02.GZ43_506492 | U81144 | Caenorhabditis elegans non-alpha | 3.20E−07 |
| | | | nicotinic acetylcholine receptor subunit | |
| | | | precursor (unc-29) gene, complete cds | |
| 356 | 3553.N04.GZ43_506524 | AE006296 | Lactococcus lactis subsp. lactis IL1403 | 2.00E−06 |
| | | | section 58 of 218 of the complete | |
| | | | genome | |
| 357 | 3553.N07.GZ43_506572 | AK026258 | Homo sapiens cDNA: FLJ22605 fis, | 1.00E−06 |
| | | | clone HSI04743 | |
| 358 | 3553.N08.GZ43_506588 | U05822 | Human proto-oncogene BCL3 gene, | 1.90E−14 |
| | | | exon 2 | |
| 359 | 3553.O07.GZ43_506573 | X97196 | D. melanogaster X gene | 4.00E−06 |
| 360 | 3553.O18.GZ43_506749 | AE001146 | Borrelia burgdorferi (section 32 of 70) of | 1.60E−05 |
| | | | the complete genome | |
| 361 | 3553.O23.GZ43_506829 | X63509 | Mus musculus partial L1 gene, exons 2-4 | 6.00E−06 |
| 362 | 3553.P03.GZ43_506510 | M24901 | Rabbit pulmonary surfactant-associated | 4.20E−07 |
| | | | protein (SP-B) mRNA, complete cds | |
| 363 | 3553.P05.GZ43_506542 | AF239156 | Homo sapiens peptide deformylase-like | 1.00E−06 |
| | | | protein mRNA, complete cds | |
| 364 | 3553.P12.GZ43_506654 | AF283753 | Acipenser persicus isolate cw203 | 3.90E−07 |
| | | | cytochrome b gene, partial cds; | |
| | | | mitochondrial gene for mitochondrial | |
| | | | product | |
| 365 | 3553.P18.GZ43_506750 | AK021312 | Mus musculus 13 days embryo stomach | 3.50E−08 |
| | | | cDNA, RIKEN full-length enriched | |
| | | | library, clone: D530039A21, full insert | |
| | | | sequence | |
| 366 | 3553.P21.GZ43_506798 | AB044136 | Homo sapiens genomic DNA, clone: #7 | 4.00E−06 |
| 367 | 3556.A03.GZ43_506879 | X61084 | C. griseus rhodopsin gene for opsin | 4.30E−05 |
| | | | protein | |
| 368 | 3556.A06.GZ43_506927 | L46904 | Homo sapiens (subclone 4_c6 from P1 | 1.20E−08 |
| | | | H22) DNA sequence | |
| 369 | 3556.B06.GZ43_506928 | AK002041 | Homo sapiens cDNA FLJ11179 fis, | 1.40E−07 |
| | | | clone PLACE1007450 | |
| 370 | 3556.B09.GZ43_506976 | U88832 | Human groucho protein homolog (AES) | 7.00E−07 |
| | | | gene, exons 2-7 and complete cds | |
| 371 | 3556.B10.GZ43_506992 | M11925 | Influenza | 5.00E−06 |
| | | | A/chicken/Pennsylvania/8125/83 | |
| | | | (H5N2) neuraminidase (NA) gene, | |
| | | | complete cds | |
| 372 | 3556.B14.GZ43_507056 | Z80218 | Caenorhabditis elegans cosmid F52D4, | 2.20E−05 |
| | | | complete sequence | |
| 373 | 3556.C13.GZ43_507041 | AF348512 | Mus musculus polyamine-modulated | 8.00E−06 |
| | | | factor-1 gene, exons 2 through 5 and | |
| | | | complete cds | |
| 374 | 3556.C15.GZ43_507073 | X82013 | S. cerevisiae mRNA for SUL1 | 3.00E−06 |
| 375 | 3556.C18.GZ43_507121 | Z23973 | H. sapiens (D7S660) DNA segment | 5.00E−06 |
| | | | containing (CA) repeat; clone | |
| | | | AFM277vd5; single read | |
| 376 | 3556.C24.GZ43_507217 | AE001381 | Plasmodium falciparum chromosome 2, | 6.90E−07 |
| | | | section 18 of 73 of the complete | |
| | | | sequence | |
| 377 | 3556.D15.GZ43_507074 | U48288 | Rattus norvegicus A-kinase anchoring | 5.50E−07 |
| | | | protein AKAP 220 mRNA, complete cds | |
| 378 | 3556.D20.GZ43_507154 | AF092684 | Neochlamisus scabripennis haplotype | 4.00E−07 |
| | | | 113 cytochrome oxidase I (COI) gene, | |
| | | | mitochondrial gene encoding | |
| | | | mitochondrial protein, partial cds | |
| 379 | 3556.D23.GZ43_507202 | X16416 | Human c-abl mRNA encoding p150 | 2.25E−04 |
| | | | protein | |
| 380 | 3556.E13.GZ43_507043 | AL049948 | Homo sapiens mRNA; cDNA | 6.60E−08 |
| | | | DKFZp564K0222 (from clone | |
| | | | DKFZp564K0222) | |
| 381 | 3556.E24.GZ43_507219 | Z57634 | H. sapiens CpG island DNA genomic | 8.70E−07 |
| | | | Msel fragment, clone 187e9, forward | |
| | | | read cpg187e9.ft1a | |
| 382 | 3556.F10.GZ43_506996 | AF025409 | Homo sapiens zinc transporter 4 (ZNT4) | 3.90E−34 |
| | | | mRNA, complete cds | |
| 383 | 3556.G15.GZ43_507077 | X15407 | Maize pseudo-Gpa2 pseudogene for | 3.40E−05 |
| | | | glyceraldehyde-3-phosphate | |
| | | | dehydrogenase subunit A | |
| 384 | 3556.H01.GZ43_506854 | AF269443 | Staphylococcus epidermidis strain SR1 | 3.00E−06 |
| | | | clone step.1003h04 genomic sequence | |
| 385 | 3556.H02.GZ43_506870 | U31465 | Kluyveromyces lactis telomerase RNA | 3.00E−06 |
| | | | component (TER1) gene, complete | |
| | | | sequence | |
| 386 | 3556.H12.GZ43_507030 | Z68886 | Human DNA sequence from cosmid | 1.70E−07 |
| | | | L21F12, Huntington's Disease Region, | |
| | | | chromosome 4p16.3 | |
| 387 | 3556.H20.GZ43_507158 | AB034628 | Equus caballus microsatellite TKY319, | 1.70E−07 |
| | | | TKY320 DNA | |
| 388 | 3556.I02.GZ43_506871 | AL390767 | Human DNA sequence from clone RP1- | 2.00E−06 |
| | | | 68P15 on chromosome 11p13-14.2 | |
| | | | Contains GSSs and ESTs. Contains part | |
| | | | of a novel gene, complete sequence | |
| | | | [Homo sapiens] | |
| 389 | 3556.I14.GZ43_507063 | U34042 | Mus musculus mammalian tolloid-like | 1.50E−05 |
| | | | protein mRNA, complete cds | |
| 390 | 3556.J05.GZ43_506920 | U31465 | Kluyveromyces lactis telomerase RNA | 2.00E−06 |
| | | | component (TER1) gene, complete | |
| | | | sequence | |
| 391 | 3556.J07.GZ43_506952 | AL359621 | Homo sapiens mRNA; cDNA | 2.00E−06 |
| | | | DKFZp434M1631 (from clone | |
| | | | DKFZp434M1631) | |
| 392 | 3556.J14.GZ43_507064 | M81830 | Human somatostatin receptor isoform 2 | 1.00E−06 |
| | | | (SSTR2) gene, complete cds | |
| 393 | 3556.J16.GZ43_507096 | Y15484 | Canis familiaris gene encoding retinal | 2.90E−08 |
| | | | guanylate cyclase E | |
| 394 | 3556.K04.GZ43_506905 | U88832 | Human groucho protein homolog (AES) | 8.00E−07 |
| | | | gene, exons 2-7 and complete cds | |
| 395 | 3556.K12.GZ43_507033 | AP001419 | Homo sapiens genomic DNA, | 1.00E−06 |
| | | | chromosome 21q22.2, clone: PAC24K9, | |
| | | | LB7T-ERG region, complete sequence | |
| 396 | 3556.K13.GZ43_507049 | AK023589 | Homo sapiens cDNA FLJ13527 fis, | 2.00E−06 |
| | | | clone PLACE1006076 | |
| 397 | 3556.K17.GZ43_507113 | X71634 | D. bifasciata P-Transposon | 3.00E−06 |
| 398 | 3556.L08.GZ43_506970 | X02367 | Glaucoma chattoni rDNA 3′ NTS | 8.20E−08 |
| 399 | 3556.L09.GZ43_506986 | AF154329 | Pisum sativum MAP kinase PsMAPK2 | 4.10E−07 |
| | | | (Mapk2) mRNA, complete cds | |
| 400 | 3556.L16.GZ43_507098 | AB041791 | Homo sapiens HSPDE10A gene for | 3.10E−08 |
| | | | phosphodiesterase 10A1 (PDE10A1), | |
| | | | exon 17 | |
| 401 | 3556.L23.GZ43_507210 | M23720 | Rat carboxypeptidase (CA2) gene, exon | 5.00E−06 |
| | | | 10 | |
| 402 | 3556.M02.GZ43_506875 | U91963 | Human tolloid-like protein (TLL) | 1.40E−05 |
| | | | mRNA, complete cds | |
| 403 | 3556.M11.GZ43_507019 | X16353 | R. rickettsii ompB gene for outer | 7.60E−05 |
| | | | membrane protein B | |
| 404 | 3556.M23.GZ43_507211 | X93496 | H. sapiens TRAP gene, 5′ flanking region | 5.60E−23 |
| 405 | 3556.N02.GZ43_506876 | U26458 | Snakehead retrovirus (SnRV), complete | 3.20E−05 |
| | | | genome | |
| 406 | 3556.N04.GZ43_506908 | L39064 | Homo sapiens interleukin 9 receptor | 4.00E−09 |
| | | | precursor (IL9R) gene, complete cds | |
| 407 | 3556.N05.GZ43_506924 | M63437 | Chicken KLG gene, complete cds | 2.00E−06 |
| 408 | 3556.N06.GZ43_506940 | AF327424 | Arabidopsis thaliana unknown protein | 2.00E−07 |
| | | | (T14P1.19/At2g45010) mRNA, partial | |
| | | | cds | |
| 409 | 3556.N21.GZ43_507180 | AB022157 | Mus musculus Cctd gene for chaperonin | 4.00E−06 |
| | | | containing TCP-1 delta subunit, | |
| | | | complete cds | |
| 410 | 3556.O08.GZ43_506973 | X00171 | Vibrio cholera toxin (ctx) operon DNA | 7.00E−06 |
| | | | sequence from strain 2125 | |
| 411 | 3556.O13.GZ43_507053 | U41106 | Caenorhabditis elegans cosmid W06A11 | 1.20E−05 |
| 412 | 3556.P07.GZ43_506958 | M15085 | T. brucei expressed copy of the ILTat 1.3 | 1.10E−07 |
| | | | variable surface glycoprotein gene, 5′ | |
| | | | flank | |
| 413 | 3559.A04.GZ43_507279 | AE006824 | Sulfolobus solfataricus section 183 of | 4.70E−05 |
| | | | 272 of the complete genome | |
| 414 | 3559.A20.GZ43_507535 | X71787 | A. thaliana AAP2 mRNA for amino acid | 2.00E−06 |
| | | | permease | |
| 415 | 3559.A24.GZ43_507599 | X56494 | H. sapiens M gene for M1-type and M2- | 1.80E−05 |
| | | | type pyruvate kinase | |
| 416 | 3559.B04.GZ43_507280 | AJ251550 | Homo sapiens partial AK155 gene for | 2.50E−05 |
| | | | AK155 protein, exons 1-3 and joined | |
| | | | CDS | |
| 417 | 3559.B06.GZ43_507312 | AF077344 | Homo sapiens cartilage-derived C-type | 5.80E−05 |
| | | | lectin (CLECSF1) gene, exons 1 and 2 | |
| 418 | 3559.B08.GZ43_507344 | D50552 | Xenopus laevis xSox12 mRNA for | 4.00E−07 |
| | | | XSOX12, complete cds | |
| 419 | 3559.B10.GZ43_507376 | L76259 | Homo sapiens PTS gene, complete cds | 9.00E−06 |
| 420 | 3559.B18.GZ43_507504 | M29109 | D. discoideum actin M6 gene, 5′ flank | 3.40E−07 |
| 421 | 3559.C06.GZ43_507313 | X99910 | C. carpio mRNA transcription factor, | 1.60E−05 |
| | | | ovx1 | |
| 422 | 3559.D21.GZ43_507554 | AK022877 | Homo sapiens cDNA FLJ12815 fis, | 2.00E−06 |
| | | | clone NT2RP2002546 | |
| 423 | 3559.E06.GZ43_507315 | U97408 | Caenorhabditis elegans cosmid F48A9 | 3.00E−06 |
| 424 | 3559.E09.GZ43_507363 | L40489 | Ureaplasma urealyticum UreA (ureA), | 3.00E−07 |
| | | | UreB (ureB), UreC (ureC), UreE (ureE), | |
| | | | UreF (ureF), and UreG (ureG) genes, | |
| | | | complete cds; UreD (ureD) gene, partial | |
| | | | cds; and unknown gene | |
| 425 | 3559.E20.GZ43_507539 | AF113521 | Zea mays putative transcription factor | 8.20E−08 |
| | | | mRNA sequence | |
| 426 | 3559.F07.GZ43_507332 | AF109377 | Mus musculus ldlBp (LDLB) mRNA, | 4.30E−05 |
| | | | complete cds | |
| 427 | 3559.F17.GZ43_507492 | U11292 | Human Ki nuclear autoantigen mRNA, | 6.40E−07 |
| | | | complete cds | |
| 428 | 3559.H09.GZ43_507366 | X13414 | Murine I gene for MHC class II(Ia) | 9.00E−06 |
| | | | associated invariant chain | |
| 429 | 3559.H22.GZ43_507574 | U61402 | Streptococcus thermophilus GalR (galR), | 1.00E−06 |
| | | | galactokinase (galK) and gal-1-P | |
| | | | uridylyltransferase (galT) genes, | |
| | | | complete cds | |
| 430 | 3559.H24.GZ43_507606 | U67594 | Methanococcus jannaschii section 136 of | 3.40E−05 |
| | | | 150 of the complete genome | |
| 431 | 3559.I05.GZ43_507303 | X97289 | S. salar genes encoding alpha-globin and | 7.00E−06 |
| | | | beta-globin, clone 6 | |
| 432 | 3559.J04.GZ43_507288 | L10709 | Human constitutive endothelial nitric | 8.90E−12 |
| | | | oxide synthase gene, exons 25 and 26 | |
| | | | and complete cds | |
| 433 | 3559.J20.GZ43_507544 | U67559 | Methanococcus jannaschii section 101 of | 5.70E−05 |
| | | | 150 of the complete genome | |
| 434 | 3559.K16.GZ43_507481 | Z48955 | D. virginiana partial LINE-1 repetitive | 2.40E−08 |
| | | | DNA and putative RT | |
| 435 | 3559.K17.GZ43_507497 | AC004497 | Homo sapiens chromosome 21, P1 clone | 4.00E−06 |
| | | | LBNL#6 (LBNL H10), complete | |
| | | | sequence | |
| 436 | 3559.L01.GZ43_507242 | X58774 | Herpesvirus saimiri sRNA1, sRNA2, | 1.00E−06 |
| | | | sRNA3 and sRNA4 genes for small | |
| | | | viral RNAs | |
| 437 | 3559.L14.GZ43_507450 | X67774 | C. upsaliensis (LMG 8854) 23S rRNA | 1.30E−05 |
| | | | gene | |
| 438 | 3559.L19.GZ43_507530 | Z57634 | H. sapiens CpG island DNA genomic | 7.70E−07 |
| | | | Mse1 fragment, clone 187e9, forward | |
| | | | read cpg187e9.ft1a | |
| 439 | 3559.M02.GZ43_507259 | AF042834 | Homo sapiens phosphodiesterase delta | 1.30E−05 |
| | | | subunit gene, exons 2, 3 and 4 | |
| 440 | 3559.M09.GZ43_507371 | U07628 | Caenorhabditis elegans N2 APX-1 (apx- | 2.00E−06 |
| | | | 1) mRNA, complete cds | |
| 441 | 3559.N05.GZ43_507308 | Z24259 | H. sapiens (D19S417) DNA segment | 3.70E−07 |
| | | | containing (CA) repeat; clone | |
| | | | AFM304zg1; single read | |
| 442 | 3559.N18.GZ43_507516 | S75829 | {dinucleotide repeats, microsatellite | 1.90E−07 |
| | | | marker} [Dryobalanops lanceolata, | |
| | | | Genomic, 230 nt] | |
| 443 | 3559.N21.GZ43_507564 | AL353948 | Homo sapiens mRNA; cDNA | 5.30E−07 |
| | | | DKFZp761P0114 (from clone | |
| | | | DKFZp761P0114) | |
| 444 | 3559.O01.GZ43_507245 | AL110269 | Homo sapiens mRNA; cDNA | 1.60E−17 |
| | | | DKFZp564A122 (from clone | |
| | | | DKFZp564A122); partial cds | |
| 445 | 3559.O05.GZ43_507309 | Y08695 | Clostridium tertium nanH gene | 7.40E−07 |
| 446 | 3559.O07.GZ43_507341 | AJ249489 | Xenopus laevis partial mRNA for | 5.40E−07 |
| | | | putative olfactory receptor (xb6 gene) | |
| 447 | 3559.O20.GZ43_507549 | X02886 | Human gene for T-cell receptor alpha | 2.00E−06 |
| | | | chain J region | |
| 448 | 3559.P10.GZ43_507390 | X66030 | Homo sapiens partial ufo gene encoding | 4.90E−07 |
| | | | tyrosine kinase receptor | |
| 449 | 3559.P15.GZ43_507470 | Z16777 | H. sapiens (D2S139) DNA segment | 4.00E−06 |
| | | | containing (CA) repeat; clone | |
| | | | AFM177xh4; single read | |
| 450 | 3559.P18.GZ43_507518 | AJ228072 | Nicotiana benthamiana DNA for Tnt1 | 2.80E−07 |
| | | | retrotransposable element, isolate ben15 | |
| 451 | 3559.P24.GZ43_507614 | U32372 | Rattus norvegicus tyrosine-ester | 4.90E−07 |
| | | | sulfotransferase mRNA, complete cds | |
| 452 | 3562.A01.GZ43_507615 | AE000496 | Escherichia coli K12 MG1655 section | 1.56E−04 |
| | | | 386 of 400 of the complete genome | |
| 453 | 3562.A15.GZ43_507839 | AF068289 | Homo sapiens HDCMD34P mRNA, | 6.60E−11 |
| | | | complete cds | |
| 454 | 3562.B22.GZ43_507952 | AK014534 | Mus musculus 0 day neonate skin | 1.10E−07 |
| | | | cDNA, RIKEN full-length enriched | |
| | | | library, clone: 4631424J17, full insert | |
| | | | sequence | |
| 455 | 3562.C23.GZ43_507969 | L01787 | Ascaris suum phosphoenolpyruvate | 3.70E−07 |
| | | | carboxykinase (PEPCK) gene, complete | |
| | | | cds | |
| 456 | 3562.D10.GZ43_507762 | X54061 | D. melanogaster mRNA coding for a | 6.60E−07 |
| | | | 205K microtubule-associated protein | |
| | | | (MAP) | |
| 457 | 3562.E01.GZ43_507619 | M29812 | Homo sapiens Ig H-chain V71-4 | 1.50E−05 |
| | | | (IGH@) gene, partial cds | |
| 458 | 3562.E03.GZ43_507651 | X03729 | Vaccinia virus late gene cluster from | 2.63E−04 |
| | | | central portion of genome containing the | |
| | | | L65 gene locus | |
| 459 | 3562.E12.GZ43_507795 | M29694 | B. licheniformis RNA polymerase sigma- | 1.60E−05 |
| | | | 30 factor (spo0H) gene, complete cds | |
| 460 | 3562.F19.GZ43_507908 | AE006216 | Pasteurella multocida PM70 section 183 | 2.40E−05 |
| | | | of 204 of the complete genome | |
| 461 | 3562.F20.GZ43_507924 | M91004 | Rabbit endothelial leukocyte adhesion | 2.00E−06 |
| | | | molecule I (ELAM1), complete cds | |
| 462 | 3562.G13.GZ43_507813 | X69818 | E. muelleri COLF1 gene for extracellular | 1.00E−06 |
| | | | matrix protein | |
| 463 | 3562.G19.GZ43_507909 | AK019034 | Mus musculus 10 day old male pancreas | 1.00E−05 |
| | | | cDNA, RIKEN full-length enriched | |
| | | | library, clone: 1810049K24, full insert | |
| | | | sequence | |
| 464 | 3562.H11.GZ43_507782 | AF206598 | Algyroides fitzingeri 12S ribosomal | 1.40E−07 |
| | | | RNA gene, partial sequence; tRNA-Val | |
| | | | gene, complete sequence; and 16S | |
| | | | ribosomal RNA gene, partial sequence; | |
| | | | mitochondrial genes for mitochondrial | |
| | | | products | |
| 465 | 3562.H12.GZ43_507798 | AK002041 | Homo sapiens cDNA FLJ11179 fis, | 5.30E−07 |
| | | | clone PLACE 1007450 | |
| 466 | 3562.I01.GZ43_507623 | S39048 | knob associated histidine-rich protein | 2.00E−06 |
| | | | KAHRP {5′region} [Plasmodium | |
| | | | falciparum, Genomic, 2215 nt] | |
| 467 | 3562.I02.GZ43_507639 | AF129501 | Buchnera aphidicola natural-host | 1.60E−07 |
| | | | Diuraphis noxia acetohydroxy acid | |
| | | | synthase large subunit (ilvI) and | |
| | | | acetohydroxy acid synthase small | |
| | | | subunit (ilvH) genes, complete cds; and | |
| | | | unknown genes | |
| 468 | 3562.I13.GZ43_507815 | M26049 | Yeast (S. cerevisiae) RAD9 protein | 4.00E−06 |
| | | | (required for cell cycle arrest during | |
| | | | DNA repair) gene, complete cds | |
| 469 | 3562.I15.GZ43_507847 | AF310880 | Barbatula barbatula microsatellite Bbar5 | 1.60E−07 |
| | | | sequence | |
| 470 | 3562.J09.GZ43_507752 | AF236642 | Calothrix parietina clone 102-2A 16S-23S | 3.30E−07 |
| | | | internal transcribed spacer, complete | |
| | | | sequence; and tRNA-Ile and tRNA-Ala | |
| | | | genes, complete sequence | |
| 471 | 3562.J13.GZ43_507816 | AC010728 | Homo sapiens BAC clone RP11-258E22 | 1.30E−05 |
| | | | from Y, complete sequence | |
| 472 | 3562.K04.GZ43_507673 | S79777 | {specific DNA probe for Plasmodium | 5.40E−07 |
| | | | vivax pARC 1153} [Plasmodium vivax, | |
| | | | host = human, Genomic, 665 nt] | |
| 473 | 3562.K08.GZ43_507737 | AJ403240 | M. musculus DNA for vimentin-binding | 2.00E−06 |
| | | | fragment VimE8 | |
| 474 | 3562.L12.GZ43_507802 | AE007840 | Clostridium acetobutylicum ATCC824 | 5.80E−07 |
| | | | section 328 of 356 of the complete | |
| | | | genome | |
| 475 | 3562.N24.GZ43_507996 | AF255609 | Homo sapiens high mobility group | 2.00E−07 |
| | | | protein HMG1 gene, exons 1 and 2, | |
| | | | partial cds | |
| 476 | 3562.O11.GZ43_507789 | M15027 | Human myelin proteolipid protein gene, | 1.00E−06 |
| | | | exon 2 | |
| 477 | 3562.O18.GZ43_507901 | AL050208 | Homo sapiens mRNA; cDNA | 2.40E−07 |
| | | | DKFZp586F2323 (from clone | |
| | | | DKFZp586F2323) | |
| 478 | 3562.O20.GZ43_507933 | AY020756 | Oryza sativa microsatellite MRG3081 | 4.90E−08 |
| | | | containing (TA)X13, genomic sequence | |
| 479 | 3562.P21.GZ43_507950 | AF036318 | Skeletonema costatum cyclin (CYCL) | 7.20E−07 |
| | | | gene, partial cds | |
| 480 | 3562.P23.GZ43_507982 | AF126719 | Plasmodium falciparum cAMP- | 3.00E−06 |
| | | | dependent protein kinase (pka) gene, | |
| | | | complete cds | |
| 481 | 3565.A23.GZ43_508351 | AL122065 | Homo sapiens mRNA; cDNA | 1.50E−07 |
| | | | DKFZp434N011 (from clone | |
| | | | DKFZp434N011) | |
| 482 | 3565.B05.GZ43_508064 | AF163325 | Trichoderma harzianum mitochondrial | 1.50E−07 |
| | | | plasmid pThr1, complete plasmid | |
| | | | sequence | |
| 483 | 3565.B13.GZ43_508192 | X62689 | T. retusa DNA for brachiopod cubitus- | 9.00E−06 |
| | | | interruptus dominant (ciD) homologue | |
| 484 | 3565.B14.GZ43_508208 | M29929 | Human insulin receptor (allele 1) gene, | 4.30E−12 |
| | | | exons 14, 15, 16 and 17 | |
| 485 | 3565.C04.GZ43_508049 | AE006183 | Pasteurella multocida PM70 section 150 | 2.00E−06 |
| | | | of 204 of the complete genome | |
| 486 | 3565.C06.GZ43_508081 | S79836 | SCPx/SCP2 = sterol carrier protein | 3.00E−06 |
| | | | x/sterol carrier protein 2 {promoter} | |
| | | | [human, Genomic, 3575 nt] | |
| 487 | 3565.C17.GZ43_508257 | L13937 | Bovine phospholipase C mRNA, | 3.00E−07 |
| | | | complete cds | |
| 488 | 3565.D14.GZ43_508210 | M37818 | Human keratin (psi-K-alpha) | 3.50E−08 |
| | | | pseudogene, exons 4, 5, 6, 7 and 8, and | |
| | | | keratin (psi-K-beta) pseudogene, | |
| | | | complete cds | |
| 489 | 3565.D17.GZ43_508258 | Z19005 | C. pasteurianum gene for ferredoxin | 1.00E−06 |
| 490 | 3565.D19.GZ43_508290 | AE007758 | Clostridium acetobutylicum ATCC824 | 3.00E−06 |
| | | | section 246 of 356 of the complete | |
| | | | genome | |
| 491 | 3565.E16.GZ43_508243 | L42813 | Protopterus dolloi complete | 2.49E−04 |
| | | | mitochondrial genome | |
| 492 | 3565.G07.GZ43_508101 | U97500 | Homo sapiens butyrophilin (BT3.3) | 1.30E−05 |
| | | | gene, exons 1-4 | |
| 493 | 3565.G09.GZ43_508133 | M95098 | Bos taurus lysozyme gene (cow 2), | 1.26E−04 |
| | | | complete cds | |
| 494 | 3565.G22.GZ43_508341 | AJ400873 | Homo sapiens partial GPLD1 gene for | 1.40E−09 |
| | | | glycosylphosphatidylinositol | |
| | | | phospholipase D, exons 15-20 | |
| 495 | 3565.H06.GZ43_508086 | U67465 | Methanococcus jannaschii section 7 of | 6.10E−07 |
| | | | 150 of the complete genome | |
| 496 | 3565.H10.GZ43_508150 | M15350 | Bacillus sp. strain 170 beta-lactamase | 5.70E−08 |
| | | | gene, complete cds | |
| 497 | 3565.H11.GZ43_508166 | AB044878 | Equus caballus DNA, microsatellite | 3.20E−09 |
| | | | TKY378 | |
| 498 | 3565.H15.GZ43_508230 | AL122122 | Homo sapiens mRNA; cDNA | 5.00E−06 |
| | | | DKFZp434L098 (from clone | |
| | | | DKFZp434L098) | |
| 499 | 3565.H23.GZ43_508358 | J05492 | E. coli cytochrome O ubiquinol oxidase | 1.00E−06 |
| | | | (cyoA, cyoB, cyoC, cyoD and cyoE | |
| | | | genes, complete cds | |
| 500 | 3565.H24.GZ43_508374 | AE001417 | Plasmodium falciparum chromosome 2, | 1.70E−10 |
| | | | section 54 of 73 of the complete | |
| | | | sequence | |
| 501 | 3565.K15.GZ43_508233 | AB062985 | Macaca fascicularis brain cDNA | 6.90E−105 |
| | | | clone: QmoA-10670, full insert sequence | |
| 502 | 3565.L22.GZ43_508346 | L81801 | Homo sapiens (subclone 1_a2 from P1 | 1.30E−05 |
| | | | H31) DNA sequence, complete sequence | |
| 503 | 3565.M15.GZ43_508235 | X08038 | Methanobacterium thermoautotrophicum | 1.10E−05 |
| | | | rpoT, rpoU, rpoV and rpoX genes for | |
| | | | RNA polymerase subunits A, B′, B″ and C | |
| 504 | 3565.M20.GZ43_508315 | Z93381 | Caenorhabditis elegans cosmid F28G4, | 1.20E−05 |
| | | | complete sequence | |
| 505 | 3565.N12.GZ43_508188 | M21573 | Salmon (S. salar) growth hormone gene, | 5.70E−05 |
| | | | complete cds | |
| 506 | 3565.N13.GZ43_508204 | AK001163 | Homo sapiens cDNA FLJ10301 fis, | 5.20E−08 |
| | | | clone NT2RM2000032 | |
| 507 | 3565.N19.GZ43_508300 | AF321321 | Homo sapiens dopamine transporter | 2.00E−06 |
| | | | (SLC6A3) gene, exon 15 and complete | |
| | | | cds | |
| 508 | 3565.O02.GZ43_508029 | X59773 | Pisum sativum mRNA for P protein, a | 1.30E−05 |
| | | | part of glycine cleavage complex | |
| 509 | 3565.O03.GZ43_508045 | Z27113 | H. sapiens gene for RNA polymerase II | 2.00E−15 |
| | | | subunit 14.4 kD | |
| 510 | 3565.O07.GZ43_508109 | X96607 | M. musculus IgH 3′ alpha enhancer DNA | 6.40E−05 |
| 511 | 3565.O15.GZ43_508237 | Z35484 | Thermoanaerobacter sp. ATCC53627 | 3.00E−06 |
| | | | cgtA gene | |
| 512 | 3565.P03.GZ43_508046 | U11292 | Human Ki nuclear autoantigen mRNA, | 6.40E−07 |
| | | | complete cds | |
| 513 | 3565.P09.GZ43_508142 | X56261 | Yeast PPH1 gene for protein | 1.00E−06 |
| | | | phosphatase 2A | |
| 514 | 3565.P22.GZ43_508350 | AE007790 | Clostridium acetobutylicum ATCC824 | 3.00E−06 |
| | | | section 278 of 356 of the complete | |
| | | | genome | |
| 515 | 3565.P24.GZ43_508382 | X61146 | N. tabacum NTP303 pollen specific | 2.70E−05 |
| | | | mRNA | |
| 516 | 3568.A10.GZ43_508545 | U46925 | Arabidopsis thaliana GTP-binding | 3.00E−06 |
| | | | protein ATGB2 mRNA, complete cds | |
| 517 | 3568.B02.GZ43_508418 | U83640 | Mus caroli Sp100 gene, exons 3 and 4 | 1.90E−08 |
| 518 | 3568.B05.GZ43_508466 | BC008293 | Homo sapiens, Similar to RIKEN cDNA | 3.20E−16 |
| | | | A430101B06 gene, clone MGC: 13017 | |
| | | | IMAGE: 3537789, mRNA, complete cds | |
| 519 | 3568.C22.GZ43_508739 | AF280797 | Homo sapiens NPC-related protein | 1.00E−06 |
| | | | NAG73 mRNA, complete cds | |
| 520 | 3568.D23.GZ43_508756 | AK022922 | Homo sapiens cDNA FLJ12860 fis, | 8.00E−06 |
| | | | clone NT2RP2003559 | |
| 521 | 3568.E17.GZ43_508661 | AF068294 | Homo sapiens HDCMB45P mRNA, | 5.30E−09 |
| | | | partial cds | |
| 522 | 3568.E20.GZ43_508709 | AE006417 | Lactococcus lactis subsp. lactis IL1403 | 1.10E−05 |
| | | | section 179 of 218 of the complete | |
| | | | genome | |
| 523 | 3568.F06.GZ43_508486 | U52198 | Vibrio anguillarum flagellin E (flaE), | 2.20E−05 |
| | | | flagellin D (flaD), and flagellin B (flaB) | |
| | | | genes, complete cds, and (flaG) gene, | |
| | | | partial cds | |
| 524 | 3568.F07.GZ43_508502 | Z23599 | H. sapiens (D13S263) DNA segment | 1.90E−08 |
| | | | containing (CA) repeat; clone | |
| | | | AFM210yg11; single read | |
| 525 | 3568.F11.GZ43_508566 | AE007525 | Clostridium acetobutylicum ATCC824 | 4.20E−07 |
| | | | section 13 of 356 of the complete | |
| | | | genome | |
| 526 | 3568.F12.GZ43_508582 | D50416 | Mouse mRNA for AREC3, complete cds | 1.90E−05 |
| 527 | 3568.F22.GZ43_508742 | AF025900 | Histrionicus histrionicus CA | 7.80E−07 |
| | | | dinucleotide repeat locus Hhimicro1 | |
| 528 | 3568.G10.GZ43_508551 | U66074 | Tritrichomonas foetus putative | 9.70E−07 |
| | | | superoxide dismutase 2 (SOD2) gene, | |
| | | | complete cds | |
| 529 | 3568.G12.GZ43_508583 | AB062941 | Macaca fascicularis brain cDNA | 9.50E−47 |
| | | | clone: QflA-14927, full insert sequence | |
| 530 | 3568.G24.GZ43_508775 | L27221 | Giardia intestinalis pyruvate:flavodoxin | 3.20E−05 |
| | | | oxidoreductase and flanking genes | |
| 531 | 3568.H20.GZ43_508712 | X75887 | B. taurus Brevican mRNA | 4.70E−05 |
| 532 | 3568.J10.GZ43_508554 | AF194829 | Tetragonia tetragonioides NADH | 2.00E−06 |
| | | | dehydrogenase (ndhF) gene, partial cds; | |
| | | | chloroplast gene for chloroplast product | |
| 533 | 3568.J22.GZ43_508746 | Y11031 | C. coli pldA gene | 1.00E−06 |
| 534 | 3568.K01.GZ43_508411 | AL137751 | Homo sapiens mRNA; cDNA | 3.00E−06 |
| | | | DKFZp434I0812 (from clone | |
| | | | DKFZp434I0812); partial cds | |
| 535 | 3568.K04.GZ43_508459 | Z82295 | R. prowazekii genomic DNA fragment | 7.20E−08 |
| | | | (clone A153F) | |
| 536 | 3568.L04.GZ43_508460 | AL050105 | Homo sapiens mRNA; cDNA | 1.00E−05 |
| | | | DKFZp586H0519 (from clone | |
| | | | DKFZp586H0519); partial cds | |
| 537 | 3568.M03.GZ43_508445 | L76259 | Homo sapiens PTS gene, complete cds | 8.00E−06 |
| 538 | 3568.M13.GZ43_508605 | X61218 | M. musculus cervicolor (strain CRP) | 3.10E−09 |
| | | | Tcp-1 gene for t-complex polypeptide 1, | |
| | | | exons 8-10 | |
| 539 | 3568.N11.GZ43_508574 | AL079296 | Homo sapiens mRNA full length insert | 2.00E−06 |
| | | | cDNA clone EUROIMAGE 609395 | |
| 540 | 3568.O17.GZ43_508671 | AF078848 | Homo sapiens BUP mRNA, complete | 9.50E−09 |
| | | | cds | |
| 541 | 3568.P04.GZ43_508464 | AB041548 | Mus musculus brain cDNA, clone | 5.00E−06 |
| | | | MNCb-3816, similar to AF171875 g1- | |
| | | | related zinc finger protein (Mus | |
| | | | musculus) | |
| 542 | 3568.P18.GZ43_508688 | AL358951 | Human DNA sequence from clone RP3- | 3.00E−07 |
| | | | 456L16 on chromosome 6, complete | |
| | | | sequence [Homo sapiens] | |
| 543 | 3568.P19.GZ43_508704 | U43542 | Nicotiana tabacum diphenol oxidase | 2.00E−06 |
| | | | mRNA, complete cds | |
| 544 | 3571.A04.GZ43_508833 | AF017116 | Homo sapiens type-2 phosphatidic acid | 2.40E−07 |
| | | | phosphohydrolase (PAP2) mRNA, | |
| | | | complete cds | |
| 545 | 3571.A07.GZ43_508881 | L81867 | Homo sapiens (subclone 1_a8 from P1 | 9.00E−06 |
| | | | H54) DNA sequence, complete sequence | |
| 546 | 3571.A08.GZ43_508897 | X85041 | H. sapiens PE5L gene ALU repeat region | 2.00E−06 |
| 547 | 3571.A11.GZ43_508945 | U19361 | Petromyzon marinus neurofilament | 4.70E−08 |
| | | | subunit NF-180 mRNA, complete cds | |
| 548 | 3571.A14.GZ43_508993 | AL022342 | Human DNA sequence from clone RP1- | 7.00E−05 |
| | | | 29M10 on chromosome 20, complete | |
| | | | sequence [Homo sapiens] | |
| 549 | 3571.A22.GZ43_509121 | U09448 | Vaucheria bursata protein synthesis | 7.20E−07 |
| | | | elongation factor Tu (tufA) gene, | |
| | | | chloroplast gene encoding chloroplast | |
| | | | protein, partial cds | |
| 550 | 3571.B13.GZ43_508978 | AE002555 | Neisseria meningitidis serogroup B strain | 4.40E−05 |
| | | | MC58 section 197 of 206 of the | |
| | | | complete genome | |
| 551 | 3571.B22.GZ43_509122 | AE002555 | Neisseria meningitidis serogroup B strain | 4.50E−05 |
| | | | MC58 section 197 of 206 of the | |
| | | | complete genome | |
| 552 | 3571.C08.GZ43_508899 | AJ010154 | Saguinus oedipus msp-E1 gene | 1.10E−17 |
| 553 | 3571.D04.GZ43_508836 | AF125460 | Caenorhabditis elegans cosmid Y9D1A | 3.60E−07 |
| 554 | 3571.D07.GZ43_508884 | U51654 | Barbus barbus × Barbus meridionalis | 8.72E−02 |
| | | | microsatellite clone no. 37 | |
| 555 | 3571.E02.GZ43_508805 | AF329081 | Bos taurus AMP-activated protein kinase | 4.40E−33 |
| | | | gamma-1 (PRKAG1) gene, partial cds | |
| 556 | 3571.E10.GZ43_508933 | M96068 | Madagascar periwinkle | 3.30E−08 |
| | | | hydroxymethylglutaryl-CoA reductase | |
| | | | (HMGR) mRNA, complete cds | |
| 557 | 3571.E16.GZ43_509029 | AE006429 | Lactococcus lactis subsp. lactis IL1403 | 1.30E−05 |
| | | | section 191 of 218 of the complete | |
| | | | genome | |
| 558 | 3571.F06.GZ43_508870 | AL137296 | Homo sapiens mRNA; cDNA | 4.40E−07 |
| | | | DKFZp434M0416 (from clone | |
| | | | DKFZp434M0416) | |
| 559 | 3571.F16.GZ43_509030 | M58478 | Human cystic fibrosis transmembrane | 6.30E−05 |
| | | | conductance regulator gene, 5′ end | |
| 560 | 3571.F23.GZ43_509142 | AF038397 | Mus musculus glutaminase (Gls) gene, | 4.70E−08 |
| | | | partial 3′ sequence | |
| 561 | 3571.G22.GZ43_509127 | M80596 | Saccharomyces cerevisiae VAC1 gene | 7.00E−06 |
| | | | (required for vacuole inheritance and | |
| | | | vacuole protein sorting), complete cds | |
| 562 | 3571.G24.GZ43_509159 | Z75330 | H. sapiens mRNA for nuclear protein SA-1 | 1.00E−46 |
| 563 | 3571.H01.GZ43_508792 | U71144 | Influenza A virus H3N2 A/Akita/1/94 | 1.90E−05 |
| | | | nucleoprotein (NP) gene, complete cds | |
| 564 | 3571.H10.GZ43_508936 | AF038564 | Homo sapiens atrophin-1 interacting | 6.60E−53 |
| | | | protein 4 (AIP4) mRNA, partial cds | |
| 565 | 3571.H12.GZ43_508968 | K00131 | mouse b2 repeat sequence from clone | 3.00E−08 |
| | | | mm61 | |
| 566 | 3571.H16.GZ43_509032 | AF179564 | Homo sapiens GTF2I-like sequence | 1.20E−23 |
| | | | within duplicated segment of Williams | |
| | | | syndrome region | |
| 567 | 3571.H18.GZ43_509064 | AE000331 | Escherichia coli K12 MG1655 section | 1.45E−04 |
| | | | 221 of 400 of the complete genome | |
| 568 | 3571.I11.GZ43_508953 | U20661 | Dictyostelium discoideum unknown | 9.00E−06 |
| | | | internal repeat protein gene, complete | |
| | | | cds, and unknown orf1, orf2 and orf3 | |
| | | | genes, partial cds | |
| 569 | 3571.J07.GZ43_508890 | M58478 | Human cystic fibrosis transmembrane | 6.40E−05 |
| | | | conductance regulator gene, 5′ end | |
| 570 | 3571.J08.GZ43_508906 | AK021312 | Mus musculus 13 days embryo stomach | 3.60E−08 |
| | | | cDNA, RIKEN full-length enriched | |
| | | | library, clone: D530039A21, full insert | |
| | | | sequence | |
| 571 | 3571.J09.GZ43_508922 | X66483 | D. discoideum gp80 gene | 8.90E−07 |
| 572 | 3571.J14.GZ43_509002 | L77119 | Methanococcus jannaschii small extra- | 1.40E−05 |
| | | | chromosomal element, complete | |
| | | | sequence.˜ | |
| 573 | 3571.L01.GZ43_508796 | AK005500 | Mus musculus adult female placenta | 6.00E−06 |
| | | | cDNA, RIKEN full-length enriched | |
| | | | library, clone: 1600019O04, full insert | |
| | | | sequence | |
| 574 | 3571.M17.GZ43_509053 | AF085681 | Mus musculus tubby like protein 1 | 5.00E−06 |
| | | | (Tulp1) mRNA, complete cds | |
| 575 | 3571.M19.GZ43_509085 | D10487 | B. thermoglucosidasius gene for oligo- | 9.00E−06 |
| | | | 1,6-glucosidase | |
| 576 | 3571.M24.GZ43_509165 | M97680 | Bluetongue virus type 2 genomic RNA | 2.00E−06 |
| | | | sequence | |
| 577 | 3571.N09.GZ43_508926 | X86100 | R. norvegicus BSP gene | 3.40E−07 |
| 578 | 3571.N14.GZ43_509006 | D32007 | Mouse mRNA for a homlogue of | 1.20E−08 |
| | | | human CBFA2T1(Mtg8a), complete cds | |
| 579 | 3571.N17.GZ43_509054 | Z68755 | Human DNA sequence from cosmid | 1.70E−10 |
| | | | L118D5, Huntington's Disease Region, | |
| | | | chromosome 4p16.3 | |
| 580 | 3571.N22.GZ43_509134 | D00326 | Porcine rotavirus (strain Gottfried), VP6 | 1.00E−06 |
| | | | gene, complete cds | |
| 581 | 3571.O08.GZ43_508911 | X66483 | D. discoideum gp80 gene | 8.20E−07 |
| 582 | 3574.A20.GZ43_509473 | AJ271814 | Drosophila melanogaster mRNA for | 1.70E−07 |
| | | | meso18E protein | |
| 583 | 3574.B01.GZ43_509170 | U93261 | Homo sapiens DESP4P1 pseudogene | 1.00E−06 |
| | | | sequence | |
| 584 | 3574.B04.GZ43_509218 | Y08207 | C. elaphus mitochondrial tRNA-Thr, | 1.40E−14 |
| | | | tRNA-Pro and tRNA-Phe genes | |
| 585 | 3574.B10.GZ43_509314 | AL161991 | Homo sapiens mRNA; cDNA | 3.00E−06 |
| | | | DKFZp761C169 (from clone | |
| | | | DKFZp761C169); partial cds | |
| 586 | 3574.B14.GZ43_509378 | D79208 | Apis mellifera mRNA for alpha- | 7.00E−06 |
| | | | glucosidase, complete cds | |
| 587 | 3574.B24.GZ43_509538 | AE007758 | Clostridium acetobutylicum ATCC824 | 3.00E−06 |
| | | | section 246 of 356 of the complete | |
| | | | genome | |
| 588 | 3574.C09.GZ43_509299 | AF057708 | Populus balsamifera subsp. trichocarpa | 2.40E−07 |
| | | | PTD protein (PTD) gene, complete cds | |
| 589 | 3574.C10.GZ43_509315 | AE005602 | Escherichia coli O157:H7 EDL933 | 9.70E−05 |
| | | | genome, contig 3 of 3, section 221 of | |
| | | | 290 | |
| 590 | 3574.C12.GZ43_509347 | AJ223633 | Enterococcus faecium genes encoding | 9.50E−07 |
| | | | enterocin L50A and enterocin L50B plus | |
| | | | 5′ and 3′ flanking regions | |
| 591 | 3574.C14.GZ43_509379 | X99710 | L. lactis ORF, genes homologous to vsf-1 | 4.00E−06 |
| | | | and pepF2 and gene encoding protein | |
| | | | homologous to methyltransferase | |
| 592 | 3574.C16.GZ43_509411 | AF092920 | Chlorohydra viridissima head-activator | 3.00E−07 |
| | | | binding protein precursor (HAB) mRNA, | |
| | | | complete cds | |
| 593 | 3574.C23.GZ43_509523 | AB047856 | Oryza sativa Ub-CEP52-2 gene for | 5.00E−08 |
| | | | ubiquitin fused to ribosomal protein L40, | |
| | | | complete cds | |
| 594 | 3574.D02.GZ43_509188 | AB060225 | Macaca fascicularis brain cDNA | 570E−07 |
| | | | clone: QflA-14955, full insert sequence | |
| 595 | 3574.D12.GZ43_509348 | M58478 | Human cystic fibrosis transmembrane | 6.00E−05 |
| | | | conductance regulator gene, 5′ end | |
| 596 | 3574.E02.GZ43_509189 | L37347 | Human integral membrane protein | 2.00E−06 |
| | | | (Nramp2) mRNA, partial | |
| 597 | 3574.E03.GZ43_509205 | X05817 | Bovine papillomavirus type 4 (BPV-4) | 6.00E−06 |
| | | | genome | |
| 598 | 3574.E14.GZ43_509381 | U67507 | Methanococcus jannaschii section 49 of | 3.40E−05 |
| | | | 150 of the complete genome | |
| 599 | 3574.F10.GZ43_509318 | M24376 | Mouse zinc finger protein (krox-20) | 3.80E−08 |
| | | | gene, exon 1 | |
| 600 | 3574.F18.GZ43_509446 | AF184170 | Sparus aurata elongation factor 1-alpha | 3.40E−07 |
| | | | (EF1-alpha) mRNA, complete cds | |
| 601 | 3574.F23.GZ43_509526 | Z29486 | R. norvegicus (Sprague Dawley) mRNA | 9.00E−06 |
| | | | for AMP-activated protein kinase | |
| 602 | 3574.G07.GZ43_509271 | AF064079 | Plasmodium gallinaceum endochitinase | 1.40E−07 |
| | | | precursor, mRNA, complete cds | |
| 603 | 3574.G11.GZ43_509335 | AF032872 | Rattus norvegicus potassium channel | 7.40E−07 |
| | | | regulatory protein KChAP mRNA, | |
| | | | complete cds | |
| 604 | 3574.H07.GZ43_509272 | J04718 | Human proliferating cell nuclear antigen | 3.10E−07 |
| | | | (PCNA) gene, complete cds | |
| 605 | 3574.I02.GZ43_509193 | AF200361 | Rattus norvegicus cytochrome P450 4F1 | 1.50E−05 |
| | | | (Cyp4F1) gene, complete cds | |
| 606 | 3574.I07.GZ43_509273 | M29688 | S. cerevisiae PMS1 gene encoding DNA | 1.20E−08 |
| | | | mismatch repair protein, complete cds | |
| 607 | 3574.J11.GZ43_509338 | Z24104 | H. sapiens (D12S338) DNA segment | 3.20E−07 |
| | | | containing (CA) repeat; clone | |
| | | | AFM291wd9; single read | |
| 608 | 3574.J14.GZ43_509386 | AB008430 | Homo sapiens mRNA for CDEP, | 4.70E−05 |
| | | | complete cds | |
| 609 | 3574.J23.GZ43_509530 | AP000384 | Arabidopsis thaliana genomic DNA, | 7.10E−07 |
| | | | chromosome 3, P1 clone: MCE21 | |
| 610 | 3574.K12.GZ43_509355 | AB031814 | Mus musculus oatp2 mRNA for organic | 1.50E−05 |
| | | | anion transporting polypeptide 2, | |
| | | | complete cds | |
| 611 | 3574.K20.GZ43_509483 | AF126719 | Plasmodium falciparum cAMP- | 3.00E−06 |
| | | | dependent protein kinase (pka) gene, | |
| | | | complete cds | |
| 612 | 3574.L07.GZ43_509276 | U53400 | Rattus norvegicus chromosome 10 | 8.94E−02 |
| | | | microsatellite sequence D10Mco21 | |
| 613 | 3574.M03.GZ43_509213 | AB000404 | Rice grassy stunt virus genomic RNA6 | 5.60E−07 |
| | | | for 20.6K major nonstructural protein | |
| | | | and 36.4K protein, complete cds | |
| 614 | 3574.M23.GZ43_509533 | U18056 | Lycopersicon esculentum 1-amino- | 3.40E−07 |
| | | | cyclopropane-1-carboxylate synthase | |
| | | | (LE-ACS1A) gene, complete cds | |
| 615 | 3574.N04.GZ43_509230 | L48479 | Homo sapiens (subclone 6_h1 from P1 | 3.30E−09 |
| | | | H21) DNA sequence | |
| 616 | 3574.N10.GZ43_509326 | M58150 | Bovine lactoperoxidase (LPO) mRNA, | 3.60E−05 |
| | | | complete cds | |
| 617 | 3574.N12.GZ43_509358 | AF182950 | Homo sapiens HEX (HEX) gene, partial | 9.00E−06 |
| | | | cds and 5′ flanking sequence | |
| 618 | 3574.N20.GZ43_509486 | AE006904 | Sulfolobus solfataricus section 263 of | 3.00E−06 |
| | | | 272 of the complete genome | |
| 619 | 3574.P07.GZ43_509280 | U60232 | Homo sapiens cysteine dioxygenase | 6.30E−08 |
| | | | (CDO-1) gene, 5′ flanking region and | |
| | | | exons 1 and 2 | |
| 620 | 3574.P17.GZ43_509440 | AC002218 | Homo sapiens (subclone 2_c1 from P1 | 5.30E−08 |
| | | | H43) DNA sequence, complete sequence | |
| 621 | 3577.A06.GZ43_509633 | U28328 | Bos taurus dinucleotide repeat RM154, | 3.40E−27 |
| | | | tandem repeat region | |
| 622 | 3577.A18.GZ43_509825 | X58774 | Herpesvirus saimiri sRNA1, sRNA2, | 1.00E−06 |
| | | | sRNA3 and sRNA4 genes for small | |
| | | | viral RNAs | |
| 623 | 3577.B12.GZ43_509730 | BC008400 | Homo sapiens, postmeiotic segregation | 2.50E−05 |
| | | | increased (S. cerevisiae) 2, clone | |
| | | | IMAGE: 4273792, mRNA | |
| 624 | 3577.B15.GZ43_509778 | M61127 | Drosophila melanogaster GTP-binding | 1.10E−05 |
| | | | protein (arf-like) gene, complete cds | |
| 625 | 3577.B19.GZ43_509842 | AF135526 | Homo sapiens clone MINT26 colon | 1.00E−06 |
| | | | cancer differentially methylated CpG | |
| | | | island genomic sequence | |
| 626 | 3577.E19.GZ43_509845 | AF063864 | Schizosaccharomyces pombe essential | 1.00E−06 |
| | | | nuclear protein Mcm3p (mcm3+) gene, | |
| | | | complete cds | |
| 627 | 3577.F02.GZ43_509574 | U37434 | Danio rerio L-isoaspartate (D-aspartate) | 5.10E−08 |
| | | | O-methyltransferase (PCMT) mRNA, | |
| | | | complete cds | |
| 628 | 3577.G07.GZ43_509655 | AF001893 | Human MEN1 region clone epsilon/beta | 3.00E−06 |
| | | | mRNA, 3′ fragment | |
| 629 | 3577.G13.GZ43_509751 | M83821 | Xenopus laevis mucin B.1 consensus | 2.10E−07 |
| | | | repeat mRNA | |
| 630 | 3577.H06.GZ43_509640 | AK007565 | Mus musculus 10 day old male pancreas | 8.00E−07 |
| | | | cDNA, RIKEN full-length enriched | |
| | | | library, clone: 1810020K22, full insert | |
| | | | sequence | |
| 631 | 3577.H08.GZ43_509672 | L81912 | Homo sapiens (subclone 2_g5 from PAC | 2.40E−07 |
| | | | H74) DNA sequence, complete sequence | |
| 632 | 3577.H18.GZ43_509832 | AL157461 | Homo sapiens mRNA; cDNA | 4.00E−06 |
| | | | DKFZp434K152 (from clone | |
| | | | DKFZp434K152) | |
| 633 | 3577.I01.GZ43_509561 | U35006 | Carcharhinus plumbeus Ig lambda light | 2.00E−06 |
| | | | chain gene, complete cds | |
| 634 | 3577.I17.GZ43_509817 | AF157252 | Gongronella butleri translation | 1.00E−06 |
| | | | elongation factor 1-alpha (EF-1alpha) | |
| | | | gene, partial cds | |
| 635 | 3577.J04.GZ43_509610 | AF338249 | Sus scrofa thyroid-stimulating hormone | 2.00E−06 |
| | | | receptor mRNA, complete cds | |
| 636 | 3577.K06.GZ43_509643 | AB000264 | Bacillus firmus DNA for beta-amylase, | 5.00E−07 |
| | | | partial cds | |
| 637 | 3577.K14.GZ43_509771 | X15441 | Aspergillus nidulans mitochondrial ndhC | 1.00E−06 |
| | | | and oxiB genes for NADH | |
| | | | dehydrogenase subunit 3 and cytochrome | |
| | | | oxidase subunit II | |
| 638 | 3577.K23.GZ43_509915 | X52952 | Rat mRNA for c-mos | 3.00E−06 |
| 639 | 3577.L10.GZ43_509708 | X60578 | Hepatitis C genomic RNA for putative | 3.70E−07 |
| | | | envelope protein (RE56 isolate) | |
| 640 | 3577.N10.GZ43_509710 | Z75121 | S. cerevisiae chromosome XV reading | 4.50E−09 |
| | | | frame ORF YOR213c | |
| 641 | 3577.N14.GZ43_509774 | M90058 | Human serglycin gene, exons 1, 2, and 3 | 5.00E−06 |
| 642 | 3577.O17.GZ43_509823 | L19141 | Lupinus albus L-asparaginase gene, | 9.10E−08 |
| | | | complete cds | |
| 643 | 3577.O22.GZ43_509903 | AL031008 | Human DNA sequence from clone | 5.60E−08 |
| | | | 360A4 on chromosome 16. Contains | |
| | | | ESTs, complete sequence [Homo | |
| | | | sapiens] | |
| 644 | 3577.P02.GZ43_509584 | AK006176 | Mus musculus adult male testis cDNA, | 4.60E−08 |
| | | | RIKEN full-length enriched library, | |
| | | | clone: 1700020M10, full insert sequence | |
| 645 | 3577.P07.GZ43_509664 | U05822 | Human proto-oncogene BCL3 gene, | 2.40E−14 |
| | | | exon 2 | |
| 646 | 3577.P23.GZ43_509920 | AJ010341 | Homo sapiens PISSLRE gene, exons 1, | 1.00E−11 |
| | | | 2, and 3 and joined CDS | |
| 647 | 3580.A04.GZ43_509985 | AJ010213 | Mus musculus beta-dystrobrevin gene, | 8.20E−07 |
| | | | exon 10 | |
| 648 | 3580.A09.GZ43_510065 | AB037862 | Homo sapiens mRNA for KIAA1441 | 6.30E−15 |
| | | | protein, partial cds | |
| 649 | 3580.A13.GZ43_510129 | U17832 | Symploce pallens mitochondrion 16S | 7.80E−07 |
| | | | ribosomal RNA, partial sequence | |
| 650 | 3580.A14.GZ43_510145 | X89414 | A. thaliana DNA for pyrroline-5- | 6.00E−06 |
| | | | carboxylase synthetase gene | |
| 651 | 3580.B01.GZ43_509938 | U67487 | Methanococcus jannaschii section 29 of | 9.00E−05 |
| | | | 150 of the complete genome | |
| 652 | 3580.C01.GZ43_509939 | X14898 | Hamster p7 preinsertion DNA | 2.00E−06 |
| 653 | 3580.C03.GZ43_509971 | X76302 | H. sapiens RY-1 mRNA for putative | 3.70E−07 |
| | | | nucleic acid binding protein | |
| 654 | 3580.C05.GZ43_510003 | Z22923 | M. musculus alpha2 (IX) collagen gene, | 1.60E−05 |
| | | | complete CDS | |
| 655 | 3580.D07.GZ43_510036 | AB062941 | Macaca fascicularis brain cDNA | 9.80E−22 |
| | | | clone: QflA-14927, full insert sequence | |
| 656 | 3580.D22.GZ43_510276 | M84136 | Flaveria chloraefolia flavonol 4′- | 4.00E−06 |
| | | | sulfotransferase mRNA, complete cds | |
| 657 | 3580.E02.GZ43_509957 | AE001002 | Archaeoglobus fulgidus section 105 of | 3.90E−05 |
| | | | 172 of the complete genome | |
| 658 | 3580.E08.GZ43_510053 | U48431 | Drosophila pseudoobscura alpha- | 3.00E−06 |
| | | | amylase (Amy3) pseudogene, complete | |
| | | | cds | |
| 659 | 3580.E10.GZ43_510085 | Z64717 | H. sapiens CpG island DNA genomic | 9.60E−19 |
| | | | Mse1 fragment, clone 161e9, forward | |
| | | | read cpg161e9.ft1a | |
| 660 | 3580.E19.GZ43_510229 | M64984 | Candida tropicalis open reading frame | 2.00E−06 |
| | | | DNA sequence | |
| 661 | 3580.E21.GZ43_510261 | M84136 | Flaveria chloraefolia flavonol 4′- | 5.00E−06 |
| | | | sulfotransferase mRNA, complete cds | |
| 662 | 3580.E23.GZ43_510293 | AB033570 | Eptatretus burgeri hgPTPR5a mRNA, | 2.00E−06 |
| | | | partial cds | |
| 663 | 3580.G03.GZ43_509975 | Y14277 | Drosophila melanogaster mRNA for | 1.10E−05 |
| | | | nuclear protein SA | |
| 664 | 3580.G13.GZ43_510135 | AK018491 | Mus musculus adult male colon cDNA, | 4.40E−08 |
| | | | RIKEN full-length enriched library, | |
| | | | clone: 9030408N04, full insert sequence | |
| 665 | 3580.G14.GZ43_510151 | AF142660 | Lama glama microsatellite LCA90 | 2.60E−07 |
| | | | sequence | |
| 666 | 3580.G18.GZ43_510215 | D86226 | Spinacia oleracea DNA for nitrate | 2.60E−05 |
| | | | reductase, complete cds | |
| 667 | 3580.G19.GZ43_510231 | U60502 | Glycine max actin (Soy119) gene, partial | 7.00E−06 |
| | | | cds | |
| 668 | 3580.G20.GZ43_510247 | D38524 | Human mRNA for 5′-nucleotidase | 4.80E−11 |
| 669 | 3580.G24.GZ43_510311 | AF084480 | Mus musculus Williams-Beuren | 5.00E−06 |
| | | | syndrome deletion transcript 9 homolog | |
| | | | (Wbscr9) mRNA, complete cds | |
| 670 | 3580.H12.GZ43_510120 | X78423 | D. carota (Queen Anne's Lace) Inv*Dc3 | 4.00E−06 |
| | | | gene, 4444 bp | |
| 671 | 3580.H16.GZ43_510184 | Y13786 | Homo sapiens mRNA for meltrin- | 4.50E−10 |
| | | | beta/ADAM 19 homologue | |
| 672 | 3580.H22.GZ43_510280 | X62578 | C. caldarium plastid genes ompR′, psbD, | 2.50E−05 |
| | | | psbC, rps16 and groEL | |
| 673 | 3580.I06.GZ43_510025 | X51344 | Spiroplasma virus (SpV1-R8A2 B) | 4.70E−07 |
| | | | complete genome | |
| 674 | 3580.I08.GZ43_510057 | X02761 | Human mRNA for fibronectin (FN | 1.02E−04 |
| | | | precursor) | |
| 675 | 3580.I18.GZ43_510217 | BC007856 | Homo sapiens, clone MGC: 14337 | 2.60E−10 |
| | | | IMAGE: 4298428, mRNA, complete cds | |
| 676 | 3580.J10.GZ43_510090 | AF068206 | Rangifer tarandus microsatellite | 4.40E−11 |
| | | | NVHRT16 sequence | |
| 677 | 3580.J12.GZ43_510122 | AE008323 | Agrobacterium tumefaciens strain C58 | 9.30E−05 |
| | | | linear chromosome, section 127 of 187 | |
| | | | of the complete sequence | |
| 678 | 3580.J18.GZ43_510218 | AF222689 | Homo sapiens protein arginine N- | 1.50E−05 |
| | | | methyltransferase 1 (HRMT1L2) gene, | |
| | | | complete cds, alternatively spliced | |
| 679 | 3580.J20.GZ43_510250 | M31651 | Homo sapiens sex hormone-binding | 3.80E−07 |
| | | | globulin (SHBG) gene, complete cds | |
| 680 | 3580.J21.GZ43_510266 | AB054062 | Pagrus major lpl mRNA for lipoprotein | 3.00E−06 |
| | | | lipase, complete cds | |
| 681 | 3580.K03.GZ43_509979 | AE007607 | Clostridium acetobutylicum ATCC824 | 5.00E−05 |
| | | | section 95 of 356 of the complete | |
| | | | genome | |
| 682 | 3580.K05.GZ43_510011 | Z15027 | H. sapiens HLA class III DNA | 3.70E−08 |
| 683 | 3580.K21.GZ43_510267 | AF135826 | Mus musculus neuronal nitric oxide | 2.20E−09 |
| | | | synthase (NOS-I) gene, exon 1c and 5′- | |
| | | | flanking sequence | |
| 684 | 3580.L09.GZ43_510076 | AL049333 | Homo sapiens mRNA; cDNA | 3.40E−13 |
| | | | DKFZp564M116 (from clone | |
| | | | DKFZp564M116) | |
| 685 | 3580.L10.GZ43_510092 | AF278587 | Borrelia burgdorferi strain BC-1 outer | 2.00E−06 |
| | | | surface protein C (ospC) gene, partial | |
| | | | cds | |
| 686 | 3580.L12.GZ43_510124 | D14664 | Human mRNA for KIAA0022 gene, | 1.10E−05 |
| | | | complete cds | |
| 687 | 3580.L13.GZ43_510140 | K02269 | Human ERV3 (endogenous retrovirus 3) | 3.30E−07 |
| | | | gag gene | |
| 688 | 3580.L17.GZ43_510204 | U60232 | Homo sapiens cysteine dioxygenase | 2.00E−07 |
| | | | (CDO-1) gene, 5′ flanking region and | |
| | | | exons 1 and 2 | |
| 689 | 3580.M01.GZ43_509949 | U53400 | Rattus norvegicus chromosome 10 | 4.54E−01 |
| | | | microsatellite sequence D10Mco21 | |
| 690 | 3580.M16.GZ43_510189 | AE006406 | Lactococcus lactis subsp. lactis IL1403 | 3.00E−06 |
| | | | section 168 of 218 of the complete | |
| | | | genome | |
| 691 | 3580.M17.GZ43_510205 | AF348584 | Arabidopsis thaliana unknown protein | 6.70E−07 |
| | | | (T8K14.7) mRNA, complete cds | |
| 692 | 3580.M18.GZ43_510221 | X69908 | H. sapiens gene for mitochondrial ATP | 1.00E−05 |
| | | | synthase c subunit (P2 form) | |
| 693 | 3580.M23.GZ43_510301 | M17326 | Mouse endogenous murine leukemia | 9.00E−06 |
| | | | virus polytropic provirus DNA, complete | |
| | | | cds | |
| 694 | 3580.N10.GZ43_510094 | AF103970 | Lasioglossum rohweri cytochrome | 1.00E−06 |
| | | | oxidase I (COI) gene, mitochondrial | |
| | | | gene encoding mitochondrial protein, | |
| | | | partial cds | |
| 695 | 3580.N11.GZ43_510110 | Z80362 | H. sapiens HLA-DRB pseudogene, exon | 6.10E−11 |
| | | | 1; | |
| 696 | 3580.N14.GZ43_510158 | AB014462 | Xenopus laevis XNLRR-1 mRNA, | 1.60E−05 |
| | | | complete cds | |
| 697 | 3580.N15.GZ43_510174 | AF164381 | Anomochloa marantoidea maturase | 1.00E−06 |
| | | | (matK) gene, complete cds; chloroplast | |
| | | | gene for chloroplast product | |
| 698 | 3580.N23.GZ43_510302 | AB047880 | Macaca fascicularis brain cDNA, | 2.00E−06 |
| | | | clone: QnpA-14303 | |
| 699 | 3580.O02.GZ43_509967 | X55948 | H. aspersa cytoplasmic intermediate | 4.00E−06 |
| | | | filament gene exons 2 to 6 | |
| 700 | 3580.O06.GZ43_510031 | L34649 | Homo sapiens platelet/endothelial cell | 4.00E−06 |
| | | | adhesion molecule-1 (PECAM-1) gene, | |
| | | | exon 14 | |
| 701 | 3580.O07.GZ43_510047 | Z30183 | H. sapiens mig-5 gene | 3.00E−05 |
| 702 | 3580.O08.GZ43_510063 | AF101385 | Homo sapiens ribosomal protein L11 | 1.80E−08 |
| | | | gene, complete cds | |
| 703 | 3580.P04.GZ43_510000 | AC016707 | Homo sapiens BAC clone RP11-221K4 | 1.80E−08 |
| | | | from Y, complete sequence | |
| 704 | 3580.P05.GZ43_510016 | AF055482 | Thermotoga neapolitana galactose | 8.00E−07 |
| | | | utilization operon, complete sequence | |
| 705 | 3580.P14.GZ43_510160 | AF009133 | Rattus norvegicus CD94 (Cd94) mRNA, | 7.50E−08 |
| | | | complete cds | |
| 706 | 3580.P19.GZ43_510240 | Y15176 | Human papillomavirus type 80 E6, E7, | 7.00E−06 |
| | | | E1, E2, E4, L2, and L1 genes | |
| 707 | 3583.B06.GZ43_510402 | X51398 | Chlamydomonas moewusii chloroplast | 3.00E−06 |
| | | | DNA for ORF 563 and transfer RNA- | |
| | | | Thr | |
| 708 | 3583.B07.GZ43_510418 | U39382 | Hexachaeta amabilis 16S ribosomal | 5.50E−08 |
| | | | RNA gene, mitochondrial gene encoding | |
| | | | mitochondrial RNA, partial sequence | |
| 709 | 3583.B10.GZ43_510466 | S45332 | erythropoietin receptor [human, | 3.90E−10 |
| | | | placental, Genomic, 8647 nt] | |
| 710 | 3583.B11.GZ43_510482 | AC006623 | Caenorhabditis elegans clone C52E2, | 4.00E−06 |
| | | | complete sequence | |
| 711 | 3583.D15.GZ43_510548 | AF242297 | Homo sapiens phosducin-like protein | 3.80E−08 |
| | | | gene, promoter and exon 1 | |
| 712 | 3583.D22.GZ43_510660 | Z23548 | H. sapiens (D10S540) DNA segment | 3.20E−07 |
| | | | containing (CA) repeat; clone | |
| | | | AFM205xe11; single read | |
| 713 | 3583.E11.GZ43_510485 | X69737 | E. esula chloroplast rbcL gene for | 1.30E−08 |
| | | | ribulose-1,5-biphosphate-carboxylase | |
| | | | and promoter region | |
| 714 | 3583.E13.GZ43_510517 | AB007856 | Homo sapiens KIAA0396 mRNA, | 2.20E−05 |
| | | | partial cds | |
| 715 | 3583.E15.GZ43_510549 | X74131 | H. nelsoni small subunit ribosomal RNA | 7.00E−06 |
| 716 | 3583.E17.GZ43_510581 | AE006633 | Streptococcus pyogenes M1 GAS strain | 2.40E−07 |
| | | | SF370, section 162 of 167 of the | |
| | | | complete genome | |
| 717 | 3583.F24.GZ43_510694 | J02846 | Human tissue factor gene, complete cds | 7.40E−07 |
| 718 | 3583.G09.GZ43_510455 | X88789 | P. sativum mRNA for starch synthase | 2.10E−05 |
| | | | (2035 bp) | |
| 719 | 3583.G16.GZ43_510567 | AK000735 | Homo sapiens cDNA FLJ20728 fis, | 4.70E−07 |
| | | | clone HEP11763 | |
| 720 | 3583.G17.GZ43_510583 | AK026822 | Homo sapiens cDNA: FLJ23169 fis, | 2.60E−05 |
| | | | clone LNG09957 | |
| 721 | 3583.G21.GZ43_510647 | U13044 | Human nuclear respiratory factor-2 | 2.00E−06 |
| | | | subunit alpha mRNA, complete cds | |
| 722 | 3583.H03.GZ43_510360 | M26222 | African green monkey origin of | 1.00E−13 |
| | | | replication (ORS9) region | |
| 723 | 3583.H12.GZ43_510504 | X01669 | Human c-k-ras oncogene exon 2 from | 3.20E−08 |
| | | | lung carcinoma pr310 | |
| 724 | 3583.H13.GZ43_510520 | AK022380 | Homo sapiens cDNA FLJ12318 fis, | 2.00E−06 |
| | | | clone MAMMA1002068 | |
| 725 | 3583.H15.GZ43_510552 | L77119 | Methanococcus jannaschii small extra- | 1.60E−05 |
| | | | chromosomal element, complete | |
| | | | sequence.˜ | |
| 726 | 3583.J02.GZ43_510346 | AJ007302 | Sus scrofa triadin gene | 1.00E−06 |
| 727 | 3583.K08.GZ43_510443 | D63902 | Mouse mRNA for estrogen-responsive | 2.50E−11 |
| | | | finger protein, complete cds | |
| 728 | 3583.K10.GZ43_510475 | U11816 | Lactobacillus strain 30A ornithine | 1.00E−05 |
| | | | decarboxylase (odci) gene, complete cds | |
| 729 | 3583.K11.GZ43_510491 | X73416 | W. suaveolens mitochondrial orf1 | 6.00E−06 |
| 730 | 3583.K14.GZ43_510539 | U04367 | Bacillus thuringiensis dakota HD511 | 1.20E−05 |
| | | | CryIII delta-endotoxin gene, partial cds | |
| 731 | 3583.K17.GZ43_510587 | AE004129 | Vibrio cholerae chromosome I, section | 8.00E−06 |
| | | | 37 of 251 of the complete chromosome | |
| 732 | 3583.K23.GZ43_510683 | AE001410 | Plasmodium falciparum chromosome 2, | 4.00E−06 |
| | | | section 47 of 73 of the complete | |
| | | | sequence | |
| 733 | 3583.L05.GZ43_510396 | X55299 | C. stercorarium celZ gene for endo-beta- | 1.00E−05 |
| | | | 1,4-glucanase (Avicelase I) | |
| 734 | 3583.L08.GZ43_510444 | AF106953 | Homo sapiens SOS1 (SOS1) gene, | 7.50E−09 |
| | | | partial cds | |
| 735 | 3583.L09.GZ43_510460 | L34842 | Soybean chloroplast phytochrome A | 2.40E−05 |
| | | | (phyA) gene, complete cds | |
| 736 | 3583.L17.GZ43_510588 | X65223 | T. rubrum mitochondrion genes for | 5.00E−06 |
| | | | cytochrome oxidase I, cytochrome | |
| | | | oxidase II, ATPase 9, NADH | |
| | | | dehydrogenase subunit 4L, NADH | |
| | | | dehydrogenase subunit 5, tRNA-Gln, | |
| | | | tRNA-Met and tRNA-Arg | |
| 737 | 3583.L21.GZ43_510652 | AF106661 | Rattus norvegicus glutathione S- | 5.00E−06 |
| | | | transferase Yb4 (GstYb4) gene, | |
| | | | complete cds | |
| 738 | 3583.M08.GZ43_510445 | BC005276 | Homo sapiens, Similar to GRO2 | 3.70E−07 |
| | | | oncogene, clone IMAGE: 4071652, | |
| | | | mRNA | |
| 739 | 3583.M10.GZ43_510477 | Y00477 | Human bone marrow serine protease | 4.70E−09 |
| | | | gene (medullasin) (leukocyte neutrophil | |
| | | | elastase gene) | |
| 740 | 3583.M13.GZ43_510525 | X73030 | S. cerevisiae YGP1 gene | 7.00E−06 |
| 741 | 3583.N09.GZ43_510462 | AK018377 | Mus musculus 16 days embryo lung | 4.60E−07 |
| | | | cDNA, RIKEN full-length enriched | |
| | | | library, clone: 8430403M08, full insert | |
| | | | sequence | |
| 742 | 3583.O03.GZ43_510367 | X72698 | P. pygmaeus ZFY gene for Y-linked Zinc | 3.00E−06 |
| | | | finger protein, final intron | |
| 743 | 3583.O11.GZ43_510495 | U40161 | Arabidopsis thaliana type 2A protein | 2.00E−06 |
| | | | serine/threonine phosphatase 55 kDa B | |
| | | | regulatory subunit mRNA, complete cds | |
| 744 | 3583.O17.GZ43_510591 | U67567 | Methanococcus jannaschii section 109 of | 2.00E−06 |
| | | | 150 of the complete genome | |
| 745 | 3583.P09.GZ43_510464 | AK021312 | Mus musculus 13 days embryo stomach | 3.60E−08 |
| | | | cDNA, RIKEN full-length enriched | |
| | | | library, clone: D530039A21, full insert | |
| | | | sequence | |
| 746 | 3583.P19.GZ43_510624 | U12920 | Caenorhabditis elegans sex | 1.60E−05 |
| | | | determination (tra-3) gene, exons 2-6 | |
| 747 | 3583.P22.GZ43_510672 | AJ133800 | Homo sapiens CPNE7 gene (partial), | 7.60E−07 |
| | | | exon 2 | |
| 748 | 3590.A12.GZ43_512274 | AF185661 | Glomus intraradices strain FL208 18S | 2.00E−06 |
| | | | ribosomal RNA, partial sequence; | |
| | | | internal transcribed spacer 1, 5.8S | |
| | | | ribosomal RNA and internal transcribed | |
| | | | spacer 2, complete sequence; 26S | |
| | | | ribosomal RNA, partial sequence | |
| 749 | 3590.B01.GZ43_512099 | M96068 | Madagascar periwinkle | 7.40E−09 |
| | | | hydroxymethylglutaryl-CoA reductase | |
| | | | (HMGR) mRNA, complete cds | |
| 750 | 3590.B16.GZ43_512339 | V01527 | Mouse gene coding for major | 2.40E−12 |
| | | | histocompatibility antigen. This is a class | |
| | | | II antigen, I-A-beta | |
| 751 | 3590.B21.GZ43_512419 | AB028983 | Homo sapiens mRNA for KIAA1060 | 1.70E−05 |
| | | | protein, partial cds | |
| 752 | 3590.C20.GZ43_512404 | D86566 | Human DNA for NOTCH4, partial cds | 3.20E−07 |
| 753 | 3590.D03.GZ43_512133 | D10371 | phocine distemper virus (PDV) genomic | 2.90E−05 |
| | | | RNA for N, P, V, C, M, F, H and L | |
| | | | protein | |
| 754 | 3590.D19.GZ43_512389 | M96163 | Mus musculus (clone 2) serum inducible | 7.80E−10 |
| | | | kinase (SNK) mRNA, mRNA sequence | |
| 755 | 3590.D23.GZ43_512453 | AF086485 | Homo sapiens full length insert cDNA | 7.70E−09 |
| | | | clone ZD93E02 | |
| 756 | 3590.E08.GZ43_512214 | AF055278 | Homo sapiens DNA repair protein | 5.90E−12 |
| | | | XRCC4 (XRCC4) gene, exon 1 | |
| 757 | 3590.E10.GZ43_512246 | AE001477 | Helicobacter pylori, strain J99 section 38 | 2.00E−06 |
| | | | of 132 of the complete genome | |
| 758 | 3590.F01.GZ43_512103 | AF080395 | Entamoeba histolytica actin binding | 2.00E−06 |
| | | | protein (abp2) mRNA, partial cds | |
| 759 | 3590.F16.GZ43_512343 | X79388 | B. subtilis (168) prkA gene | 1.20E−05 |
| 760 | 3590.G01.GZ43_512104 | U32690 | Haemophilus influenzae Rd section 5 of | 2.80E−05 |
| | | | 163 of the complete genome | |
| 761 | 3590.G02.GZ43_512120 | U68040 | Cochliobolus heterostrophus polyketide | 1.25E−04 |
| | | | synthase (PKS1) gene, complete cds | |
| 762 | 3590.H04.GZ43_512153 | X66013 | T. aestivum gene for cathepsin B (Al16) | 2.50E−07 |
| 763 | 3590.H06.GZ43_512185 | X66177 | M. musculus mRNA for Hox 2.7 protein | 8.00E−06 |
| 764 | 3590.H09.GZ43_512233 | AF012899 | Sambucus nigra ribosome inactivating | 3.40E−11 |
| | | | protein precursor mRNA, complete cds | |
| 765 | 3590.H12.GZ43_512281 | Y15724 | Homo sapiens SERCA3 gene, exons 1-7 | 2.00E−06 |
| | | | (and joined CDS) | |
| 766 | 3590.H16.GZ43_512345 | AF064079 | Plasmodium gallinaceum endochitinase | 6.70E−09 |
| | | | precursor, mRNA, complete cds | |
| 767 | 3590.I16.GZ43_512346 | L06280 | Drosophila melanogaster adenine | 4.40E−07 |
| | | | phosphoribosyltransferase (APRT) gene, | |
| | | | complete cds | |
| 768 | 3590.J01.GZ43_512107 | X69573 | T. reesei xyn1 gene, complete CDS | 1.70E−07 |
| 769 | 3590.J02.GZ43_512123 | AF092047 | Homo sapiens homeobox protein Six3 | 4.00E−06 |
| | | | (SIX3) gene, complete cds | |
| 770 | 3590.J18.GZ43_512379 | AB027966 | Schizosaccharomyces pombe gene for | 2.60E−08 |
| | | | Hypothetical protein, partial cds, | |
| | | | clone: TB89 | |
| 771 | 3590.J21.GZ43_512427 | AK014727 | Mus musculus 0 day neonate head | 7.90E−08 |
| | | | cDNA, RIKEN full-length enriched | |
| | | | library, clone: 4833419G08, full insert | |
| | | | sequence | |
| 772 | 3590.J22.GZ43_512443 | AK020136 | Mus musculus 12 days embryo male | 5.90E−08 |
| | | | wolffian duct includes surrounding | |
| | | | region cDNA, RIKEN full-length | |
| | | | enriched library, clone: 6720460K10, full | |
| | | | insert sequence | |
| 773 | 3590.K06.GZ43_512188 | AF171890 | Trimeresurus trigonocephalus | 3.00E−06 |
| | | | cytochrome b (cytb) gene, partial cds; | |
| | | | mitochondrial gene for mitochondrial | |
| | | | product | |
| 774 | 3590.K10.GZ43_512252 | U16775 | Human immunodeficiency virus type 1 | 6.00E−06 |
| | | | isolate VE6 reverse transcriptase (pol) | |
| | | | gene, partial cds | |
| 775 | 3590.K19.GZ43_512396 | U40454 | Candida albicans topoisomerase type I | 3.00E−06 |
| | | | (CATOP1) gene, complete cds | |
| 776 | 3590.L08.GZ43_512221 | U52198 | Vibrio anguillarum flagellin E (flaE), | 2.00E−05 |
| | | | flagellin D (flaD), and flagellin B (flaB) | |
| | | | genes, complete cds, and (flaG) gene, | |
| | | | partial cds | |
| 777 | 3590.L10.GZ43_512253 | U01155 | Xenopus laevis angiotensin II receptor | 4.00E−06 |
| | | | mRNA, complete cds | |
| 778 | 3590.M03.GZ43_512142 | AF252499 | Bos taurus clone MNB-88 microsatellite | 4.60E−08 |
| | | | sequence | |
| 779 | 3590.M04.GZ43_512158 | AE007607 | Clostridium acetobutylicum ATCC824 | 4.50E−05 |
| | | | section 95 of 356 of the complete | |
| | | | genome | |
| 780 | 3590.M09.GZ43_512238 | L04758 | Oryctolagus cuniculus cytochrome P-450 | 1.00E−06 |
| | | | (CYP4A4) gene, 5′ end | |
| 781 | 3590.N04.GZ43_512159 | Z82038 | C. thermosaccharolyticum etfB, etfA, | 2.00E−06 |
| | | | hbd, thlA and actA genes | |
| 782 | 3590.N19.GZ43_512399 | U15603 | Saccharomyces cerevisiae Csd3p | 4.00E−06 |
| | | | (CSD3) gene, complete cds | |
| 783 | 3590.N21.GZ43_512431 | L19535 | Drosophila subobscura sry alpha gene, | 6.00E−06 |
| | | | complete cds | |
| 784 | 3590.O08.GZ43_512224 | L36588 | Homo sapiens intron-encoded U22 small | 4.30E−07 |
| | | | nucleolar RNA (UHG) gene | |
| 785 | 3596.C02.GZ43_512500 | L14849 | Drosophila melanogaster cytoplasmic | 8.90E−09 |
| | | | protein tyrosine phosphatase (PTP61F) | |
| | | | mRNA, complete cds | |
| 786 | 3596.C20.GZ43_512788 | M60286 | Herpesvirus saimiri immediate early | 1.30E−07 |
| | | | region protein genes, complete cds | |
| 787 | 3596.C22.GZ43_512820 | X15121 | Soybean Gy1 gene for glycinin subunit | 1.00E−06 |
| | | | G1 | |
| 788 | 3596.D01.GZ43_512485 | Z78414 | Caenorhabditis elegans cosmid W09D12, | 4.00E−06 |
| | | | complete sequence | |
| 789 | 3596.D07.GZ43_512581 | M88242 | Mouse glucocortoid-regulated | 1.70E−05 |
| | | | inflammatory prostaglandin G/H | |
| | | | synthase (griPGHS) mRNA, complete | |
| | | | cds | |
| 790 | 3596.D09.GZ43_512613 | X99710 | L. lactis ORF, genes homologous to vsf-1 | 5.00E−06 |
| | | | and pepF2 and gene encoding protein | |
| | | | homologous to methyltransferase | |
| 791 | 3596.D17.GZ43_512741 | AF200361 | Rattus norvegicus cytochrome P450 4F1 | 1.40E−05 |
| | | | (Cyp4F1) gene, complete cds | |
| 792 | 3596.E08.GZ43_512598 | AF111848 | Homo sapiens PRO0529 mRNA, | 5.00E−06 |
| | | | complete cds | |
| 793 | 3596.E22.GZ43_512822 | X58178 | S. pyogenes for emm41 gene | 5.00E−06 |
| 794 | 3596.F10.GZ43_512631 | AL390161 | Homo sapiens mRNA; cDNA | 2.00E−06 |
| | | | DKFZp761P0615 (from clone | |
| | | | DKFZp761P0615) | |
| 795 | 3596.G13.GZ43_512680 | AJ000044 | Tenebrio molitor LPCP29 gene | 2.00E−06 |
| 796 | 3596.H04.GZ43_512537 | U65018 | Dictyostelium discoideum | 3.60E−07 |
| | | | mannosyltransferase gene, complete cds | |
| 797 | 3596.H10.GZ43_512633 | AF104390 | Penaeus monodon hyperglycemic | 2.00E−06 |
| | | | hormone homolog PmSGP-V precursor, | |
| | | | mRNA, complete cds | |
| 798 | 3596.H17.GZ43_512745 | D28915 | Human gene for hepatitis C-associated | 1.00E−06 |
| | | | microtubular aggregate protein p44, exon | |
| | | | 9 and complete cds | |
| 799 | 3596.H22.GZ43_512825 | AF198250 | Dictyostelium discoideum lim2 protein | 7.30E−07 |
| | | | (limB) mRNA, complete cds | |
| 800 | 3596.I06.GZ43_512570 | U32444 | Solanum lycopersicum phytochrome F | 1.10E−05 |
| | | | (PHYF) gene, partial cds | |
| 801 | 3596.I16.GZ43_512730 | U32444 | Solanum lycopersicum phytochrome F | 8.00E−06 |
| | | | (PHYF) gene, partial cds | |
| 802 | 3596.J04.GZ43_512539 | D28596 | Chicken gene for c-maf proto-oncogene | 9.30E−10 |
| | | | product c-Maf, short form complete cds | |
| | | | and long form 1st exon | |
| 803 | 3596.J13.GZ43_512683 | AB007856 | Homo sapiens KIAA0396 mRNA, | 2.40E−05 |
| | | | partial cds | |
| 804 | 3596.K14.GZ43_512700 | AC024752 | Caenorhabditis elegans cosmid Y1B5A, | 3.00E−06 |
| | | | complete sequence | |
| 805 | 3596.K15.GZ43_512716 | Y00469 | Yeast mRNA for profilin | 2.00E−06 |
| 806 | 3596.L01.GZ43_512493 | X79703 | O. aries gene for beta-casein | 4.00E−06 |
| 807 | 3596.L08.GZ43_512605 | AJ007313 | Streptomyces coelicolor sigT, trxB and | 9.80E−07 |
| | | | trxA genes, and ORF1 and ORF2 | |
| 808 | 3596.L13.GZ43_512685 | AK018239 | Mus musculus adult male medulla | 1.00E−06 |
| | | | oblongata cDNA, RIKEN full-length | |
| | | | enriched library, clone: 6330563C09, full | |
| | | | insert sequence | |
| 809 | 3596.N02.GZ43_512511 | AE001387 | Plasmodium falciparum chromosome 2, | 1.00E−06 |
| | | | section 24 of 73 of the complete | |
| | | | sequence | |
| 810 | 3596.N12.GZ43_512671 | Z12841 | O. cuniculus mRNA for phospholipase | 4.00E−06 |
| 811 | 3596.N15.GZ43_512719 | U14186 | Bos taurus general vesicular transport | 1.70E−05 |
| | | | factor p115 mRNA, complete cds | |
| 812 | 3596.N16.GZ43_512735 | U41106 | Caenorhabditis elegans cosmid W06A11 | 1.10E−05 |
| 813 | 3596.N21.GZ43_512815 | AF097717 | Homo sapiens 3′-phosphoadenosine 5′- | 1.40E−07 |
| | | | phosphosulfate synthetase (PAPSS), | |
| | | | exon 8 | |
| 814 | 3596.O10.GZ43_512640 | AE001649 | Chlamydia pneumoniae section 65 of | 1.10E−05 |
| | | | 103 of the complete genome | |
| 815 | 3596.O12.GZ43_512672 | AC006623 | Caenorhabditis elegans clone C52E2, | 4.00E−06 |
| | | | complete sequence | |
| 816 | 3596.P03.GZ43_512529 | X82317 | C. thummi CpY gene | 1.49E−03 |
| 817 | 3596.P04.GZ43_512545 | AF111855 | Agrobacterium tumefaciens RNA | 2.00E−06 |
| | | | polymerase alpha subunit (rpoA) gene, | |
| | | | complete cds | |
| 818 | 3596.P07.GZ43_512593 | L40817 | Homo sapiens muscle-specific DNase I- | 3.00E−06 |
| | | | like (DNL1L) gene, exons 1-9, complete | |
| | | | cds | |
| 819 | 3596.P08.GZ43_512609 | M14505 | Human (clone PSK-J3) cyclin-dependent | 5.00E−06 |
| | | | protein kinase mRNA, complete cds., | |
| 820 | 3596.P10.GZ43_512641 | M73770 | P. falciparum RNA polymerase III largest | 2.90E−05 |
| | | | subunit gene, complete cds | |
| 821 | 3596.P21.GZ43_512817 | S82725 | NPM/ALK = fusion gene {translocation | 1.00E−07 |
| | | | breakpoint} [human, lymphoma cells | |
| | | | SU-DHL-1, Genomic, 1679 nt] | |
| 822 | 3599.A04.GZ43_512914 | X83212 | H. sapiens tryptophan hydroxylase gene, | 5.50E−07 |
| | | | promoter region | |
| 823 | 3599.A23.GZ43_513218 | U05259 | Human MB-1 gene, complete cds | 2.10E−05 |
| 824 | 3599.B15.GZ43_513091 | AF277068 | HIV-1 clone QH0791 from Trinidad and | 6.10E−07 |
| | | | Tobago, envelope protein (env) gene, | |
| | | | complete cds | |
| 825 | 3599.B16.GZ43_513107 | M60517 | Chicken vitronectin receptor alpha | 4.00E−06 |
| | | | subunit mRNA, complete cds | |
| 826 | 3599.C03.GZ43_512900 | AB021267 | Arabidopsis thaliana copia-like | 2.00E−06 |
| | | | retrotransposon AtRE2-2 gene for | |
| | | | polyprotein, complete cds | |
| 827 | 3599.C17.GZ43_513124 | U28055 | Homo sapiens hepatocyte growth factor- | 3.00E−06 |
| | | | like protein homolog mRNA, partial cds | |
| 828 | 3599.D03.GZ43_512901 | L43550 | Buchnera aphidicola anthranilate | 3.00E−06 |
| | | | synthase small subunit (trpG) gene, | |
| | | | anthranilate synthase large subunit (trpE) | |
| | | | gene, complete cds | |
| 829 | 3599.D05.GZ43_512933 | AL023779 | S. pombe chromosome II cosmid c244 | 2.00E−06 |
| 830 | 3599.D07.GZ43_512965 | AL391223 | Human chromosome 14 DNA sequence | 5.00E−06 |
| | | | Partial sequence from BAC R- | |
| | | | 325N7_PCR1 of library RPCI-11 from | |
| | | | chromosome 14 of Homo sapiens | |
| | | | (Human), complete sequence | |
| 831 | 3599.D10.GZ43_513013 | AF064079 | Plasmodium gallinaceum endochitinase | 1.70E−07 |
| | | | precursor, mRNA, complete cds | |
| 832 | 3599.E01.GZ43_512870 | U09184 | Buchnera aphidicola ferredoxin-NADP | 9.60E−07 |
| | | | reductase (fprl) gene, partial cds; | |
| | | | anthranilate synthase large subunit (trpE) | |
| | | | and anthranilate synthase small subunit | |
| | | | (trpG) genes, complete cds; heat shock | |
| | | | protein (hslU) gene, partial cds; and | |
| | | | unknown gene | |
| 833 | 3599.E05.GZ43_512934 | X60145 | Human J-alpha segment J-alpha FR9 | 1.20E−05 |
| | | | mRNA for J-alpha region of T-cell | |
| | | | receptor | |
| 834 | 3599.F17.GZ43_513127 | U27037 | Fistulina hepatica mitochondrial small | 2.00E−06 |
| | | | subunit ribosomal RNA, mitochondrial | |
| | | | gene, partial sequence | |
| 835 | 3599.F24.GZ43_513239 | Z78414 | Caenorhabditis elegans cosmid W09D12, | 5.00E−06 |
| | | | complete sequence | |
| 836 | 3599.H05.GZ43_512937 | AF032891 | Camponotus consobrinus microsatellite- | 2.10E−08 |
| | | | containing sequence Ccon12 | |
| 837 | 3599.H23.GZ43_513225 | AB024553 | Bacillus halodurans DNA, complete and | 4.70E−07 |
| | | | partial cds, strain: C-125 | |
| 838 | 3599.J11.GZ43_513035 | AB025112 | Xenopus laevis XGC-2 mRNA for | 3.00E−06 |
| | | | guanylyl cyclase-2, complete cds | |
| 839 | 3599.K02.GZ43_512892 | AJ224474 | Borrelia burgdorferi left chromosomal | 3.00E−06 |
| | | | subtelomeric region (truA gene) | |
| 840 | 3599.K04.GZ43_512924 | X99710 | L. lactis ORF, genes homologous to vsf-1 | 5.00E−06 |
| | | | and pepF2 and gene encoding protein | |
| | | | homologous to methyltransferase | |
| 841 | 3599.K23.GZ43_513228 | AF074247 | Homo sapiens neuronal delayed-rectifier | 8.00E−07 |
| | | | voltage-gated potassium channel splice | |
| | | | variant (KCNQ2) mRNA, complete cds | |
| 842 | 3599.L04.GZ43_512925 | X59773 | Pisum sativum mRNA for P protein, a | 1.40E−05 |
| | | | part of glycine cleavage complex | |
| 843 | 3599.L15.GZ43_513101 | U34282 | Rattus norvegicus fast skeletal muscle | 2.00E−06 |
| | | | sarcoplasmic reticulum Ca-ATPase | |
| | | | (SERCA1) gene, 5′-flanking sequence | |
| 844 | 3599.M04.GZ43_512926 | AK018953 | Mus musculus adult male testis cDNA, | 2.30E−11 |
| | | | RIKEN full-length enriched library, | |
| | | | clone: 1700111D04, full insert sequence | |
| 845 | 3599.M22.GZ43_513214 | AB052179 | Macaca fascicularis brain cDNA, | 4.70E−07 |
| | | | clone: QnpA-21934 | |
| 846 | 3599.M24.GZ43_513246 | AE003394 | Drosophila melanogaster genomic | 7.30E−07 |
| | | | scaffold 142000013386028, complete | |
| | | | sequence | |
| 847 | 3599.N09.GZ43_513007 | X16362 | Rat SPI-2 serine protease inhibitor gene | 1.19E−04 |
| 848 | 3599.N16.GZ43_513119 | X92421 | X. laevis mRNA for RNA helicase p54 | 3.00E−06 |
| 849 | 3599.N20.GZ43_513183 | M59447 | Drosophila melanogaster Sex-lethal | 2.00E−06 |
| | | | (Sx1) mRNA, complete cds | |
| 850 | 3599.N24.GZ43_513247 | AC005485 | Homo sapiens PAC clone RP5-998M2 | 2.00E−07 |
| | | | from 7q33-q35, complete sequence | |
| 851 | 3599.O06.GZ43_512960 | AJ131667 | Escherichia coli plasmid pSFO157 | 2.00E−06 |
| 852 | 3599.O17.GZ43_513136 | X96607 | M. musculus IgH 3′ alpha enhancer DNA | 8.10E−05 |
| 853 | 3599.P05.GZ43_512945 | X77111 | N. tabacum chi-V gene | 1.50E−07 |
| 854 | 3602.A09.GZ43_513378 | AF015303 | Xenopus laevis small GTPase Ran | 1.10E−05 |
| | | | binding protein 1 mRNA, complete cds | |
| 855 | 3602.B18.GZ43_513523 | L18892 | Tetrahymena thermophila histone | 5.70E−07 |
| | | | (H2A.1) gene, complete cds | |
| 856 | 3602.B21.GZ43_513571 | BC005233 | Homo sapiens, clone MGC: 12257 | 1.60E−10 |
| | | | IMAGE: 3950129, mRNA, complete cds | |
| 857 | 3602.B22.GZ43_513587 | X71765 | P. falciparum gene for Ca2+ —ATPase | 1.00E−06 |
| 858 | 3602.C24.GZ43_513620 | AL080106 | Homo sapiens mRNA; cDNA | 2.00E−06 |
| | | | DKFZp566O053 (from clone | |
| | | | DKFZp566O053) | |
| 859 | 3602.D06.GZ43_513333 | AF098970 | Phaseolus vulgaris NBS-LRR-like | 1.70E−07 |
| | | | protein cD7 (CO-2) mRNA, partial cds | |
| 860 | 3602.D11.GZ43_513413 | M59770 | P. falciparum calmodulin gene, complete | 2.20E−07 |
| | | | cds | |
| 861 | 3602.E04.GZ43_513302 | X53582 | Zea mays ZMPMS1 gene for 19 kDa | 1.30E−05 |
| | | | zein protein | |
| 862 | 3602.E06.GZ43_513334 | L38718 | Providencia stuartii (clone pSK.aarP) | 7.90E−07 |
| | | | transcriptional activator (aarP) gene, | |
| | | | complete cds | |
| 863 | 3602.E13.GZ43_513446 | U58106 | Blomia tropicalis allergen mRNA, | 1.70E−07 |
| | | | complete cds | |
| 864 | 3602.E21.GZ43_513574 | M15085 | T. brucei expressed copy of the ILTat 1.3 | 2.90E−07 |
| | | | variable surface glycoprotein gene, 5′ | |
| | | | flank | |
| 865 | 3602.F12.GZ43_513431 | X64802 | H. sapiens F8 mRNA for Interleukin-1- | 3.40E−58 |
| | | | like species | |
| 866 | 3602.G03.GZ43_513288 | AF036148 | Danio rerio NeuroD (nrd) mRNA, | 2.00E−06 |
| | | | complete cds | |
| 867 | 3602.G17.GZ43_513512 | U41106 | Caenorhabditis elegans cosmid W06A11 | 1.30E−05 |
| 868 | 3602.I07.GZ43_513354 | AF000941 | Mus musculus DNAse I hypersensitive | 1.20E−05 |
| | | | sites 2-6 of locus control region (LCR) | |
| | | | for T-cell receptor alpha chain (TCRa) | |
| | | | gene | |
| 869 | 3602.I11.GZ43_513418 | AL133620 | Homo sapiens mRNA; cDNA | 3.00E−06 |
| | | | DKFZp434F0621 (from clone | |
| | | | DKFZp434F0621) | |
| 870 | 3602.I15.GZ43_513482 | U23479 | Dictyostelium discoideum | 8.00E−07 |
| | | | phosphatidylinositol 4-kinase (PIK4) | |
| | | | mRNA, complete cds | |
| 871 | 3602.J13.GZ43_513451 | AK025319 | Homo sapiens cDNA: FLJ21666 fis, | 3.30E−07 |
| | | | clone COL08915 | |
| 872 | 3602.K03.GZ43_513292 | X85811 | S. cerevisiae tRNA-Leu, and ORF's | 1.10E−05 |
| | | | N2212, N2215, N2219, N2223, N2227, | |
| | | | N2231 | |
| 873 | 3602.K06.GZ43_513340 | AF133052 | Walleye epidermal hyperplasia virus | 4.00E−06 |
| | | | type 2 long terminal repeat, complete | |
| | | | sequence; gag polyprotein (gag-pol) | |
| | | | gene, complete cds; pol polyprotein | |
| | | | (gag-pol) gene, partial cds; envelope | |
| | | | polyprotein (env) and cyclin D homolog | |
| | | | genes, complete cds; and unkn> | |
| 874 | 3602.L20.GZ43_513565 | M62717 | Human CSP-B gene flanking sequence | 1.10E−05 |
| 875 | 3602.N03.GZ43_513295 | Z81126 | Caenorhabditis elegans cosmid T22E6, | 5.70E−05 |
| | | | complete sequence | |
| 876 | 3602.N06.GZ43_513343 | U62503 | Human OBR gene, intron sequence | 1.00E−06 |
| | | | immediately adjacent to the 5′ end of | |
| | | | coding exon 17 | |
| 877 | 3605.A15.gz43_513858 | Z46507 | Bovine herpesvirus type 4 genomic DNA | 5.00E−06 |
| | | | region (V.TEST) | |
| 878 | 3605.C16.gz43_513876 | AF282517 | Homo sapiens clone 10ptel_c6t7 | 9.40E−08 |
| | | | sequence | |
| 879 | 3605.E19.gz43_513926 | Z22923 | M. musculus alpha2 (IX) collagen gene, | 2.10E−05 |
| | | | complete CDS | |
| 880 | 3605.G13.gz43_513832 | AJ132752 | Gadus morhua mRNA for beta2- | 1.30E−05 |
| | | | microglobulin, clone b3 | |
| 881 | 3605.H10.gz43_513785 | AF257480 | Rana temporaria microsatellite SB80 | 4.10E−09 |
| | | | sequence | |
| 882 | 3605.H21.gz43_513961 | X63507 | M. musculus HOX-3.5 gene | 7.80E−05 |
| 883 | 3605.I19.gz43_513930 | AK002100 | Homo sapiens cDNA FLJ11238 fis, | 3.30E−11 |
| | | | clone PLACE1008532 | |
| 884 | 3605.J16.gz43_513883 | AF039197 | Gallus gallus Pax-9 gene, putative 5′ | 1.00E−07 |
| | | | regulatory sequence | |
| 885 | 3605.K19.gz43_513932 | X63853 | S. cerevisiae MAT locus genes BUD5, | 8.00E−06 |
| | | | mat-alpha1, mat-alpha2, YCR724 and | |
| | | | YCR725 | |
| 886 | 3605.M17.gz43_513902 | M30931 | Simian immunodeficiency virus (SIV) | 3.70E−05 |
| | | | proviral, complete genome | |
| 887 | 3605.N04.gz43_513695 | AF169388 | Mus musculus alpha 4 collagen IV | 8.90E−05 |
| | | | (Col4a4) mRNA, complete cds | |
| 888 | 3605.N09.gz43_513775 | AF029111 | Adelius sp. 16S ribosomal RNA gene, | 2.80E−07 |
| | | | mitochondrial gene for mitochondrial | |
| | | | RNA, partial sequence | |
| 889 | 3605.N12.gz43_513823 | BC000358 | Homo sapiens, protein kinase, AMP- | 3.90E−47 |
| | | | activated, gamma 1 non-catalytic | |
| | | | subunit, clone MGC: 8666 | |
| | | | IMAGE: 2964434, mRNA, complete cds | |
| 890 | 3605.N16.gz43_513887 | X95301 | D. rerio mRNA for HER-5 protein | 1.00E−06 |
| 891 | 3608.B06.gz43_514099 | X00004 | .taurus gene encoding pituitary | 6.30E−08 |
| | | | glycoprotein hormone alpha subunit, | |
| | | | exons 3 & 4 | |
| 892 | 3608.B12.gz43_514195 | X00525 | Mouse 28S ribosomal RNA | 3.10E−13 |
| 893 | 3608.B24.gz43_514387 | AF269848 | Staphylococcus epidermidis strain SR1 | 2.00E−06 |
| | | | clone step.1026e06 genomic sequence | |
| 894 | 3608.C18.gz43_514292 | BC000387 | Homo sapiens, U6 snRNA-associated | 2.50E−10 |
| | | | Sm-like protein, clone MGC: 8433 | |
| | | | IMAGE: 2821171, mRNA, complete cds | |
| 895 | 3608.E17.gz43_514278 | BC008245 | Homo sapiens, clone IMAGE: 3875012, | 1.00E−06 |
| | | | mRNA | |
| 896 | 3608.E20.gz43_514326 | U86646 | Ailurus fulgens beta casein gene, exon 7, | 4.70E−07 |
| | | | partial cds | |
| 897 | 3608.F13.gz43_514215 | AF125672 | Homo sapiens silencing mediator of | 2.00E−06 |
| | | | retinoic acid and thyroid hormone | |
| | | | receptor extended isoform (SMRTE) | |
| | | | mRNA, complete cds | |
| 898 | 3608.G09.gz43_514152 | AE001066 | Archaeoglobus fulgidus section 41 of | 4.00E−06 |
| | | | 172 of the complete genome | |
| 899 | 3608.H05.gz43_514089 | AJ224981 | Mus musculus calpain 3 gene, exon 1 | 3.00E−06 |
| 900 | 3608.H14.gz43_514233 | AE007394 | Streptococcus pneumoniae section 77 of | 3.20E−05 |
| | | | 194 of the complete genome | |
| 901 | 3608.H18.gz43_514297 | Z36046 | S. cerevisiae chromosome II reading | 7.00E−06 |
| | | | frame ORF YBR177c | |
| 902 | 3608.J17.gz43_514283 | AF024648 | Arabidopsis thaliana receptor-like | 8.00E−06 |
| | | | serine/threonine kinase (RKF1) mRNA, | |
| | | | complete cds | |
| 903 | 3608.J24.gz43_514395 | AJ002258 | Rattus Norvegicus mRNA for Prx3A | 3.60E−07 |
| | | | protein | |
| 904 | 3608.K03.gz43_514060 | M83199 | Simmondsia chinensis stearoyl-acyl | 2.50E−07 |
| | | | carrier protein desaturase mRNA, | |
| | | | complete cds | |
| 905 | 3608.K14.gz43_514236 | AK026999 | Homo sapiens cDNA: FLJ23346 fis, | 2.00E−06 |
| | | | clone HEP13716 | |
| 906 | 3608.L07.gz43_514125 | M32684 | Homo sapiens ITGB3 gene, intron 13, | 3.60E−07 |
| | | | fragment B, partial sequence | |
| 907 | 3608.L14.gz43_514237 | Z34845 | H. sapiens serotonin transporter gene | 8.60E−07 |
| 908 | 3608.N09.gz43_514159 | AK022341 | Homo sapiens cDNA FLJ12279 fis, | 2.00E−06 |
| | | | clone MAMMA1001743, weakly similar | |
| | | | to Y BOX BINDING PROTEIN-1 | |
| 909 | 3608.N19.gz43_514319 | M15085 | T. brucei expressed copy of the ILTat 1.3 | 7.80E−08 |
| | | | variable surface glycoprotein gene, 5′ | |
| | | | flank | |
| 910 | 3608.N20.gz43_514335 | AF026169 | Homo sapiens SALF (SALF) mRNA, | 1.00E−05 |
| | | | complete cds | |
| 911 | 3608.O04.gz43_514080 | U85193 | Human nuclear factor I-B2 (NFIB2) | 7.10E−07 |
| | | | mRNA, complete cds | |
| 912 | 3608.P22.gz43_514369 | AF124241 | Callerya australis chloroplast tRNA-Leu | 3.90E−07 |
| | | | (trnL) gene, intron sequence | |
| 913 | 3611.A17.gz43_514658 | X01412 | Drosophila melanogaster genes for | 2.00E−06 |
| | | | tRNA-Val and tRNA-Pro (90BC tRNA | |
| | | | locus) | |
| 914 | 3611.B11.gz43_514563 | AL049938 | Homo sapiens mRNA; cDNA | 9.80E−10 |
| | | | DKFZp564P1916 (from clone | |
| | | | DKFZp564P1916); partial cds | |
| 915 | 3611.B16.gz43_514643 | M86514 | Rat proline-rich protein mRNA, 3′ end | 1.30E−05 |
| 916 | 3611.C09.gz43_514532 | U55950 | Pleurodeles waltl cytochrome b (CYT-b) | 2.00E−06 |
| | | | gene, mitochondrial gene encoding | |
| | | | mitochondrial protein, partial cds | |
| 917 | 3611.E07.gz43_514502 | AF261009 | Lethrinus miniatus clone 89rte, | 1.70E−12 |
| | | | microsatellite sequence | |
| 918 | 3611.E12.gz43_514582 | M60200 | Rat vitamin D binding protein gene, | 1.50E−05 |
| | | | exons 5 and 6 | |
| 919 | 3611.E20.gz43_514710 | BC002458 | Homo sapiens, clone IMAGE: 3343171, | 2.00E−06 |
| | | | mRNA, partial cds | |
| 920 | 3611.F15.gz43_514631 | U28328 | Bos taurus dinucleotide repeat RM154, | 4.30E−27 |
| | | | tandem repeat region | |
| 921 | 3611.H10.gz43_514553 | AE003147 | Drosophila melanogaster genomic | 6.00E−07 |
| | | | scaffold 142000013385388, complete | |
| | | | sequence | |
| 922 | 3611.H22.gz43_514745 | X16135 | Human mRNA for novel heterogeneous | 7.00E−06 |
| | | | nuclear RNP protein, L protein | |
| 923 | 3611.I04.gz43_514458 | AK001460 | Homo sapiens cDNA FLJ10598 fis, | 5.10E−44 |
| | | | clone NT2RP2004841 | |
| 924 | 3611.I13.gz43_514602 | M58380 | Arabidopsis thaliana peroxidase (neutral, | 3.00E−06 |
| | | | prxCa) gene, complete cds | |
| 925 | 3611.J04.gz43_514459 | S81486 | p53 {alternatively spliced, intron 9} | 1.20E−07 |
| | | | [human, Genomic Mutant, 133 nt] | |
| 926 | 3611.J15.gz43_514635 | AC008240 | Leishmania major chromosome 22 clone | 4.90E−05 |
| | | | L9259 strain Friedlin, complete sequence | |
| 927 | 3611.J17.gz43_514667 | Z17425 | Lilium speciosum for two putative cds's | 8.90E−07 |
| 928 | 3611.J22.gz43_514747 | U60736 | Human IgHC locus intergenic sequence | 4.60E−07 |
| 929 | 3611.K01.gz43_514412 | AE001377 | Plasmodium falciparum chromosome 2, | 3.00E−06 |
| | | | section 14 of 73 of the complete | |
| | | | sequence | |
| 930 | 3611.K12.gz43_514588 | X02367 | Glaucoma chattoni rDNA 3′ NTS | 9.80E−08 |
| 931 | 3611.L22.gz43_514749 | U19361 | Petromyzon marinus neurofilament | 5.40E−08 |
| | | | subunit NF-180 mRNA, complete cds | |
| 932 | 3611.M18.gz43_514686 | X95301 | D. rerio mRNA for HER-5 protein | 1.00E−06 |
| 933 | 3611.M24.gz43_514782 | AF010239 | Caenorhabditis elegans glutathione S- | 7.70E−07 |
| | | | transferase (CeGST1) mRNA, complete | |
| | | | cds | |
| 934 | 3611.N01.gz43_514415 | L19300 | Staphylococcus aureus DNA sequence | 1.00E−06 |
| | | | encoding three ORFs, complete cds; | |
| | | | prophage phi-11 sequence homology, 5′ | |
| | | | flank | |
| 935 | 3611.N09.gz43_514543 | U50382 | Danio rerio beta and alpha globin genes, | 7.00E−06 |
| | | | partial cds | |
| 936 | 3611.O16.gz43_514656 | AB056785 | Macaca fascicularis brain cDNA | 6.60E−07 |
| | | | clone: QnpA-11655, full insert sequence | |
| 937 | 3611.P08.gz43_514529 | AK026905 | Homo sapiens cDNA: FLJ23252 fis, | 8.00E−06 |
| | | | clone COL04668 | |
| 938 | 3614.C18.gz43_515060 | AF239178 | Paracoccidioides brasiliensis lon | 5.00E−06 |
| | | | proteinase gene, complete cds; nuclear | |
| | | | gene for mitochondrial product | |
| 939 | 3614.D14.gz43_514997 | AB017511 | Hydra magnipapillata mRNA for PLC- | 1.20E−05 |
| | | | betaH1, complete cds | |
| 940 | 3614.D21.gz43_515109 | L10713 | Pig trinucleotide repeat | 1.80E−05 |
| 941 | 3614.E06.gz43_514870 | X99739 | M. musculus mRNA for UBC9 protein, | 9.10E−07 |
| | | | containing ubiquitin box | |
| 942 | 3614.F22.gz43_515127 | AK021490 | Homo sapiens cDNA FLJ11428 fis, | 2.00E−06 |
| | | | clone HEMBA1001071, highly similar | |
| | | | to PROCOLLAGEN ALPHA 1(III) | |
| | | | CHAIN PRECURSOR | |
| 943 | 3614.G20.gz43_515096 | M86514 | Rat prolin-rich protein mRNA, 3′ end | 1.30E−05 |
| 944 | 3614.H09.gz43_514921 | AF068289 | Homo sapiens HDCMD34P mRNA, | 6.60E−11 |
| | | | complete cds | |
| 945 | 3614.H22.gz43_515129 | X62423 | P. falciparum pol delta gene for DNA | 4.00E−06 |
| | | | polymerase delta | |
| 946 | 3614.J07.gz43_514891 | X81027 | H. sapiens tal-1 DNA | 1.30E−05 |
| 947 | 3614.K22.gz43_515132 | X63073 | Pseudanabaena sp. cpeBA operon | 1.60E−05 |
| | | | encoding phycoerythrin beta and alpha | |
| | | | subunits | |
| 948 | 3614.L13.gz43_514989 | V01561 | Mouse dispersed repetitive DNA | 3.00E−06 |
| | | | sequences of the R-family and simple | |
| | | | sequence DNA; member of the B1 | |
| | | | family of mouse dispersed repetitive | |
| | | | DNA sequences | |
| 949 | 3614.M08.gz43_514910 | AF272983 | Homo sapiens SRC tyrosine kinase gene, | 4.00E−06 |
| | | | exons 1alpha and 1a, alternatively | |
| | | | spliced | |
| 950 | 3614.O02.gz43_514816 | X58913 | Mitochondrion Drosophila eugracilis | 8.50E−08 |
| | | | ND2 and COI genes (partial) and genes | |
| | | | for tRNA-Trp, tRNA-Tyr, and tRNA- | |
| | | | Cys | |
| 951 | 3614.O07.gz43_514896 | AL031538 | S. pombe chromosome III cosmid c1906 | 9.80E−07 |
| 952 | 3614.O16.gz43_515040 | AB056785 | Macaca fascicularis brain cDNA | 2.00E−06 |
| | | | clone: QnpA-11655, full insert sequence | |
| 953 | 3614.P11.gz43_514961 | X91656 | M. musculus Srp20 gene | 4.60E−05 |
| 954 | 3614.P16.gz43_515041 | Z58907 | H. sapiens CpG island DNA genomic | 3.20E−70 |
| | | | Mse1 fragment, clone 116a6, forward | |
| | | | read cpg116a6.ft1a | |
| 955 | 3617.B16.gz43_515411 | AF098275 | Homo sapiens PSI2TOM20 pseudogene, | 1.10E−67 |
| | | | complete sequence | |
| 956 | 3617.C21.gz43_515492 | AJ009913 | Bos taurus plp gene | 3.40E−05 |
| 957 | 3617.F10.gz43_515319 | L07487 | Bradyrhizobium japonicum heme-copper | 6.70E−05 |
| | | | oxidase subunit I homolog (fixN), | |
| | | | cytochrome c (fixO), transmembrane | |
| | | | proteins (fixO and fixQ) diheme | |
| | | | cytochrome c (fixP) and fixG genes, | |
| | | | complete cds | |
| 958 | 3617.H16.gz43_515417 | X54192 | O. sativa GluB-2 gene for glutelin | 2.00E−06 |
| 959 | 3617.I01.gz43_515178 | AL513316 | Human DNA sequence from clone | 7.20E−08 |
| | | | RP11-522O3 on chromosome 10, | |
| | | | complete sequence [Homo sapiens] | |
| 960 | 3617.L16.gz43_515421 | AE007662 | Clostridium acetobutylicum ATCC824 | 3.00E−06 |
| | | | section 150 of 356 of the complete | |
| | | | genome | |
| 961 | 3617.L21.gz43_515501 | AL031538 | S. pombe chromosome III cosmid c1906 | 1.00E−06 |
| 962 | 3617.M08.gz43_515294 | X64802 | H. sapiens F8 mRNA for Interleukin-1- | 3.40E−58 |
| | | | like species | |
| 963 | 3617.M13.gz43_515374 | Z79239 | H. sapiens flow-sorted chromosome 6 | 1.10E−07 |
| | | | TaqI fragment, SC6pA26F6 | |
| 964 | 3617.N05.gz43_515247 | AF387666 | Mandrillus cytomegalovirus strain | 1.00E−06 |
| | | | OCOM6-2 glycoprotein B (gB) gene, | |
| | | | partial cds | |
| 965 | 3617.N10.gz43_515327 | AB017511 | Hydra magnipapillata mRNA for PLC- | 1.10E−05 |
| | | | betaH1, complete cds | |
| 966 | 3617.N14.gz43_515391 | AJ249346 | Mus musculus Ankrd2 gene for ankyrin | 1.00E−05 |
| | | | repeat domain 2 (stretch responsive | |
| | | | muscle), exons 1-9 | |
| 967 | 3617.N19.gz43_515471 | U27037 | Fistulina hepatica mitochondrial small | 2.00E−06 |
| | | | subunit ribosomal RNA, mitochondrial | |
| | | | gene, partial sequence | |
| 968 | 3617.P11.gz43_515345 | AK002100 | Homo sapiens cDNA FLJ11238 fis, | 1.20E−13 |
| | | | clone PLACE1008532 | |
| 969 | 3617.P12.gz43_515361 | U04860 | Rattus norvegicus Sprague-Dawley Ah | 8.00E−05 |
| | | | receptor mRNA, complete cds | |
| 970 | 3617.P13.gz43_515377 | AE007356 | Streptococcus pneumoniae section 39 of | 3.80E−05 |
| | | | 194 of the complete genome | |
| 971 | 3620.B03.gz43_515810 | AF238884 | Botrytis virus F, complete genome | 6.00E−06 |
| 972 | 3620.B24.gz43_516146 | AF244812 | Homo sapiens SCAN domain-containing | 1.30E−07 |
| | | | protein 2 (SCAND2) gene, complete cds, | |
| | | | alternatively spliced | |
| 973 | 3620.E12.gz43_515957 | X95301 | D. rerio mRNA for HER-5 protein | 1.00E−06 |
| 974 | 3620.E13.gz43_515973 | X52289 | Human (D21S167) DNA segment | 2.50E−19 |
| | | | containing (GT)19 repeat | |
| 975 | 3620.E17.gz43_516037 | AJ002414 | Arabidosis thaliana mRNA for a hnRNP- | 9.70E−08 |
| | | | like protein | |
| 976 | 3620.E19.gz43_516069 | X16982 | Drosophila melanogaster micropia- | 2.70E−07 |
| | | | Dm11 3′flanking DNA | |
| 977 | 3620.E23.gz43_516133 | Z49438 | S. cerevisiae chromosome X reading | 3.00E−06 |
| | | | frame ORF YJL163c | |
| 978 | 3620.E24.gz43_516149 | M75883 | Human sterol carrier protein X/sterol | 8.00E−06 |
| | | | carrier protein 2 mRNA, complete cds | |
| 979 | 3620.G17.gz43_516039 | U92971 | Human protease-activated receptor 3 | 3.80E−07 |
| | | | (PAR3) mRNA, complete cds | |
| 980 | 3620.G23.gz43_516135 | X66979 | X. laevis mRNA XLFLI | 1.60E−05 |
| 981 | 3620.J18.gz43_516058 | U37373 | Xenopus laevis tail-specific thyroid | 3.00E−06 |
| | | | hormone up-regulated (gene 5) mRNA, | |
| | | | complete cds | |
| 982 | 3620.K19.gz43_516075 | U31780 | Human papillomavirus type 22, complete | 5.00E−06 |
| | | | genome | |
| 983 | 3620.K24.gz43_516155 | M95627 | Homo sapiens angio-associated | 6.00E−06 |
| | | | migratory cell protein (AAMP) mRNA, | |
| | | | complete cds | |
| 984 | 3620.O23.gz43_516143 | L11172 | Plasmodium falciparum RNA | 1.00E−05 |
| | | | polymerase I gene, complete cds | |
| 985 | 3623.B07.gz43_516258 | AF132745 | Mus musculus Sox2 gene, regulatory | 7.70E−07 |
| | | | region sequence | |
| 986 | 3623.E03.gz43_516197 | X82566 | M. musculus glyT1 gene (exon 0a) | 1.80E−09 |
| 987 | 3623.E15.gz43_516389 | AF104420 | Porcine transmissible gastroenteritis | 2.90E−05 |
| | | | virus RNA dependent RNA polymerase | |
| | | | gene, partial cds; virus envelope protein | |
| | | | spike (S), envelope protein (sM), | |
| | | | envelope protein (M), and nucleoprotein | |
| | | | (N) genes, complete cds; and unknown | |
| | | | genes | |
| 988 | 3623.F03.gz43_516198 | AJ009936 | Homo sapiens mRNA for nuclear | 1.70E−05 |
| | | | hormone receptor PRR1 | |
| 989 | 3623.F20.gz43_516470 | U22657 | Mus musculus genomic locus related to | 5.80E−05 |
| | | | cellular morphology | |
| 990 | 3623.G14.gz43_516375 | AB035309 | Paramecium caudatum PcTERT mRNA | 3.00E−06 |
| | | | for telomerase reverse transcriptase, | |
| | | | complete cds | |
| 991 | 3623.H07.gz43_516264 | Z17324 | Homo sapiens of MUC1 gene encoding | 1.80E−07 |
| | | | Mucin | |
| 992 | 3623.H10.gz43_516312 | AB033070 | Homo sapiens mRNA for KIAA1244 | 2.80E−05 |
| | | | protein, partial cds | |
| 993 | 3623.H23.gz43_516520 | AF131763 | Homo sapiens clone 25232 mRNA | 1.70E−05 |
| | | | sequence | |
| 994 | 3623.I08.gz43_516281 | M60421 | Human cytochrome P450scc gene, 5′ end | 2.80E−05 |
| | | | and promoter region | |
| 995 | 3623.I11.gz43_516329 | AK013191 | Mus musculus 10, 11 days embryo | 3.00E−06 |
| | | | cDNA, RIKEN full-length enriched | |
| | | | library, clone: 2810429I04, full insert | |
| | | | sequence | |
| 996 | 3623.L05.gz43_516236 | AJ131991 | Linum usitatissimum target sequence for | 3.00E−06 |
| | | | LIS-1 insertion in P1 | |
| 997 | 3623.L24.gz43_516540 | U09377 | Arabidopsis thaliana GF14chi isoform | 3.00E−06 |
| | | | (GRF1) gene, complete cds | |
| 998 | 3623.M10.gz43_516317 | AF071743 | Homo sapiens topoisomerase II alpha | 4.00E−06 |
| | | | (TOP2A) gene, exons 25, 26, and 27 | |
| 999 | 3623.N23.gz43_516526 | U57489 | Eubacterium sp. VPI 12708 bile acid- | 3.70E−05 |
| | | | inducible operon bile acid-coenzyme A | |
| | | | ligase (baiB), BaiC, BaiD, bile acid 7- | |
| | | | alpha dehydratase (baiE), 3-alpha | |
| | | | hydroxysteroid dehydrogenase (baiA2), | |
| | | | BaiF, bile acid transporter (baiG), | |
| | | | NADH: flavin oxidoreductase (bai> | |
| 1000 | 3623.P22.gz43_516512 | U37761 | Human H1 histamine receptor gene, 5′- | 1.40E−12 |
| | | | flanking region | |
| 1001 | 3626.A10.gz43_516689 | D30745 | Xenopus laevis MRP RNA gene | 2.00E−07 |
| 1002 | 3626.C16.gz43_516787 | AF241271 | Bos taurus ZFY gene, intron | 1.60E−08 |
| 1003 | 3626.E07.gz43_516645 | AF053496 | Caenorhabditis elegans beta chain | 2.00E−06 |
| | | | spectrin homolog Sma1 (sma1) mRNA, | |
| | | | complete cds | |
| 1004 | 3626.F03.gz43_516582 | AJ009771 | Homo sapiens mRNA for putative RING | 2.00E−06 |
| | | | finger protein, partial | |
| 1005 | 3626.G01.gz43_516551 | BC010926 | Homo sapiens, Similar to H4 histone | 1.00E−43 |
| | | | family, member A, clone MGC: 13512 | |
| | | | IMAGE: 4273904, mRNA, complete cds | |
| 1006 | 3626.I20.gz43_516857 | AK025762 | Homo sapiens cDNA: FLJ22109 fis, | 5.80E−07 |
| | | | clone HEP18091 | |
| 1007 | 3626.I23.gz43_516905 | S55615 | (156) = G surface antigen {3′ region, | 3.40E−07 |
| | | | restriction fragment EG4} [Paramecium | |
| | | | primaurelia, Genomic, 407 nt] | |
| 1008 | 3626.M13.gz43_516749 | AE001398 | Plasmodium falciparum chromosome 2, | 4.00E−06 |
| | | | section 35 of 73 of the complete | |
| | | | sequence | |
| 1009 | 3626.M15.gz43_516781 | AF090925 | Homo sapiens clone HQ0452 PRO0452 | 3.10E−07 |
| | | | mRNA, partial cds | |
| 1010 | 3626.N07.gz43_516654 | Z58907 | H. sapiens CpG island DNA genomic | 2.90E−70 |
| | | | Mse1 fragment, clone 116a6, forward | |
| | | | read cpg116a6.ft1a | |
| 1011 | 3626.N24.gz43_516926 | AF041373 | Rattus norvegicus clathrin assembly | 8.90E−08 |
| | | | protein short form (CALM) mRNA, | |
| | | | complete cds | |
| 1012 | 3626.O08.gz43_516671 | D10445 | Mouse mRNA for protein C, complete | 5.00E−06 |
| | | | cds | |
| 1013 | 3626.P11.gz43_516720 | L48479 | Homo sapiens (subclone 6_h1 from P1 | 2.20E−07 |
| | | | H21) DNA sequence | |
| 1014 | 3626.P14.gz43_516768 | X15028 | Chicken hsp90 gene for 90 kDa-heat | 3.80E−05 |
| | | | shock protein 5′-end | |
| 1015 | 3629.A16.gz43_517169 | U16958 | Mus musculus pre-T cell receptor alpha- | 4.00E−06 |
| | | | type chain precursor mRNA, complete | |
| | | | cds | |
| 1016 | 3629.B14.gz43_517138 | X16982 | Drosophila melanogaster micropia- | 2.50E−07 |
| | | | Dm11 3′flanking DNA | |
| 1017 | 3629.C14.gz43_517139 | Z22537 | C. parvum precursor of oocyst wall | 5.00E−06 |
| | | | protein | |
| 1018 | 3629.E01.gz43_516933 | D00621 | Sus scrofa gene for follicle stimulation | 3.50E−05 |
| | | | hormone beta subunit, exons 1, 2, 3, | |
| | | | complete cds | |
| 1019 | 3629.E20.gz43_517237 | AE006900 | Sulfolobus solfataricus section 259 of | 9.00E−06 |
| | | | 272 of the complete genome | |
| 1020 | 3629.F24.gz43_517302 | Y10531 | Clostridium perfringens sod gene for | 2.00E−06 |
| | | | superoxide dismutase | |
| 1021 | 3629.H10.gz43_517080 | J03654 | Human immunodeficiency virus type 2, | 8.00E−06 |
| | | | isolate HIV2FG | |
| 1022 | 3629.H12.gz43_517112 | AF017266 | Danio rerio glutamate decarboxylase | 6.50E−07 |
| | | | (GAD67) mRNA, partial cds | |
| 1023 | 3629.I11.gz43_517097 | AF020810 | Salmonella enterica VirK (virK), Mig-14 | 3.00E−06 |
| | | | (mig-14), NxiA (nxiA), TctE (tctE), | |
| | | | TctD (tctD), TctC (tctC), TctB (tctB), | |
| | | | and TctA (tctA) genes, complete cds; | |
| | | | and O360 (o360) gene, partial cds | |
| 1024 | 3629.I16.gz43_517177 | AE007643 | Clostridium acetobutylicum ATCC824 | 4.40E−05 |
| | | | section 131 of 356 of the complete | |
| | | | genome | |
| 1025 | 3629.J03.gz43_516970 | AB017511 | Hydra magnipapillata mRNA for PLC- | 1.10E−05 |
| | | | betaH1, complete cds | |
| 1026 | 3629.J07.gz43_517034 | M20782 | Human alpha-2-plasmin inhibitor gene, | 2.90E−11 |
| | | | exons 2 to 5 | |
| 1027 | 3632.C11.gz43_517475 | AF026148 | Perilla frutescens beta-ketoacyl-ACP | 1.00E−06 |
| | | | synthase I (KAS I) mRNA, complete cds | |
| 1028 | 3632.C17.gz43_517571 | U50534 | Human BRCA2 region, mRNA sequence | 1.00E−05 |
| | | | CG003 | |
| 1029 | 3632.F07.gz43_517414 | M12036 | Human tyrosine kinase-type receptor | 4.70E−10 |
| | | | (HER2) gene, partial cds | |
| 1030 | 3632.G01.gz43_517319 | AC006621 | Caenorhabditis elegans cosmid C52A10, | 3.40E−05 |
| | | | complete sequence | |
| 1031 | 3632.I20.gz43_517625 | AK024381 | Homo sapiens cDNA FLJ14319 fis, | 9.00E−06 |
| | | | clone PLACE3000406 | |
| 1032 | 3632.K20.gz43_517627 | M27634 | Vaccinia virus P4a major core protein | 9.60E−05 |
| | | | gene, complete cds | |
| 1033 | 3632.M08.gz43_517437 | X75304 | H. sapiens giantin mRNA | 8.00E−06 |
| 1034 | 3632.M13.gz43_517517 | U18191 | Human HLA class I genomic survey | 3.20E−07 |
| | | | sequence | |
| 1035 | 3632.M19.gz43_517613 | AF012131 | Homo sapiens brachyury variant B | 3.70E−07 |
| | | | (TBX1) mRNA, complete cds | |
| 1036 | 3632.N13.gz43_517518 | AF287491 | Oncorhynchus mykiss MHC class I | 2.00E−06 |
| | | | heavy chain precursor (Onmy-UBA) | |
| | | | mRNA, Onmy-UBA*601 allele, | |
| | | | complete cds | |
| 1037 | 3632.N21.gz43_517646 | X62423 | P. falciparum pol delta gene for DNA | 4.00E−06 |
| | | | polymerase delta | |
| 1038 | 3632.O06.gz43_517407 | BC009868 | Homo sapiens, replication protein A3 | 1.40E−18 |
| | | | (14 kD), clone MGC: 16404 | |
| | | | IMAGE: 3940438, mRNA, complete cds | |
| 1039 | 3632.P07.gz43_517424 | AE001066 | Archaeoglobus fulgidus section 41 of | 3.00E−06 |
| | | | 172 of the complete genome | |
| 1040 | 3635.A06.gz43_517777 | AK005546 | Mus musculus adult female placenta | 1.40E−07 |
| | | | cDNA, RIKEN full-length enriched | |
| | | | library, clone: 1600027G01, full insert | |
| | | | sequence | |
| 1041 | 3635.A08.gz43_517809 | Z49280 | S. cerevisiae chromosome X reading | 6.00E−06 |
| | | | frame ORF YJL005w | |
| 1042 | 3635.A13.gz43_517889 | AF143236 | Homo sapiens apoptosis related protein | 2.00E−06 |
| | | | APR-2 mRNA, complete cds | |
| 1043 | 3635.D07.gz43_517796 | M58150 | Bovine lactoperoxidase (LPO) mRNA, | 3.10E−05 |
| | | | complete cds | |
| 1044 | 3635.F01.gz43_517702 | Y19128 | Homo sapiens enteropeptidase gene, | 3.00E−09 |
| | | | exon 6 | |
| 1045 | 3635.F06.gz43_517782 | X63073 | Pseudanabaena sp. cpeBA operon | 1.50E−05 |
| | | | encoding phycoerythrin beta and alpha | |
| | | | subunits | |
| 1046 | 3635.F10.gz43_517846 | AF107688 | Aedes aegypti clone 431 Feilai family of | 3.50E−05 |
| | | | SINES | |
| 1047 | 3635.H20.gz43_518008 | AE000613 | Helicobacter pylori 26695 section 91 of | 1.10E−05 |
| | | | 134 of the complete genome | |
| 1048 | 3635.J06.gz43_517786 | U15018 | Dugbe virus L protein gene, complete | 1.10E−05 |
| | | | cds | |
| 1049 | 3635.J09.gz43_517834 | X85444 | G. pallida repetitive DNA element | 2.10E−08 |
| 1050 | 3635.K05.gz43_517771 | AF090432 | Danio rerio serrateB mRNA, complete | 4.00E−06 |
| | | | cds | |
| 1051 | 3635.K06.gz43_517787 | AJ276631 | Capsicum annuum partial kn gene for | 6.10E−07 |
| | | | Knolle protein, promoter region | |
| 1052 | 3635.M18.gz43_517981 | AL591498 | Human DNA sequence from clone | 1.40E−05 |
| | | | RP11-113L12 on chromosome 13, | |
| | | | complete sequence [Homo sapiens] | |
| 1053 | 3635.O01.gz43_517711 | AF081788 | Homo sapiens putative spliceosome | 3.70E−30 |
| | | | associated protein mRNA, complete cds | |
| 1054 | 3635.O14.gz43_517919 | X72224 | S. cerevisiae genes HSS1, NPL4 and HSP | 6.00E−06 |
| 1055 | 3635.P17.gz43_517968 | AF242307 | Euphorbia esula sucrose transport protein | 2.90E−10 |
| | | | mRNA, complete cds | |
| 1056 | 3635.P18.gz43_517984 | AF078780 | Caenorhabditis elegans cosmid C04F2, | 1.74E−04 |
| | | | complete sequence | |
| 1057 | 3638.A02.gz43_518097 | M17988 | Spiroplasma virus 4 (SpV4) replicative | 4.00E−06 |
| | | | form, complete genome | |
| 1058 | 3638.A24.gz43_518449 | AF064079 | Plasmodium gallinaceum endochitinase | 1.60E−07 |
| | | | precursor, mRNA, complete cds | |
| 1059 | 3638.F15.gz43_518310 | AJ297538 | Homo sapiens partial RARA gene, intron 2 | 4.00E−06 |
| 1060 | 3638.H07.gz43_518184 | AK026258 | Homo sapiens cDNA: FLJ22605 fis, | 2.00E−06 |
| | | | clone HSI04743 | |
| 1061 | 3638.J09.gz43_518218 | U89651 | Homo sapiens matrix metalloproteinase | 8.10E−08 |
| | | | MMP Rasi-1 gene, promoter region | |
| 1062 | 3638.K06.gz43_518171 | AL139329 | Human DNA sequence from clone | 4.40E−11 |
| | | | RP11-228P1 on chromosome 6, | |
| | | | complete sequence [Homo sapiens] | |
| 1063 | 3638.L10.gz43_518236 | D26532 | Mouse mRNA for transcription factor | 2.00E−08 |
| | | | PEBP2aB2, complete cds | |
| 1064 | 3638.N05.gz43_518158 | X62294 | B. taurus mRNA for adrenal angiotensin | 9.00E−06 |
| | | | II type-1 receptor | |
| 1065 | 3643.D21.gz43_518788 | U17010 | Allomyces macrogynus mitochondrion | 1.80E−05 |
| | | | NADH dehydrogenase subunit 5 (nad5) | |
| | | | gene, complete cds | |
| 1066 | 3643.E24.gz43_518837 | AL022342 | Human DNA sequence from clone RP1- | 6.70E−05 |
| | | | 29M10 on chromosome 20, complete | |
| | | | sequence [Homo sapiens] | |
| 1067 | 3643.F07.gz43_518566 | M73962 | Bovine pregnancy-associated | 6.00E−06 |
| | | | glycoprotein 1 mRNA, complete cds | |
| 1068 | 3643.G20.gz43_518775 | AF191214 | Homo sapiens isovaleryl dehydrogenase | 1.00E−05 |
| | | | (IVD) gene, exons 1-3 | |
| 1069 | 3643.G24.gz43_518839 | AK025682 | Homo sapiens cDNA: FLJ22029 fis, | 6.00E−06 |
| | | | clone HEP08661 | |
| 1070 | 3643.H09.gz43_518600 | AK024381 | Homo sapiens cDNA FLJ14319 fis, | 1.70E−05 |
| | | | clone PLACE3000406 | |
| 1071 | 3643.I01.gz43_518473 | AF000306 | Brassica napus steroid sulfotransferase 2 | 3.00E−06 |
| | | | gene, complete cds | |
| 1072 | 3643.I02.gz43_518489 | X58433 | B. subtillis cad gene for lysine | 2.30E−05 |
| | | | decarboxylase | |
| 1073 | 3643.I18.gz43_518745 | M14872 | Mouse GnRH-GAP gene encoding | 4.00E−06 |
| | | | gonadotropin-releasing hormone and GnRH- | |
| | | | associated peptide (GAP) | |
| 1074 | 3643.I24.gz43_518841 | BC003813 | Mus musculus, clone MGC: 6139 | 2.30E−07 |
| | | | IMAGE: 3487295, mRNA, complete cds | |
| 1075 | 3643.K06.gz43_518555 | AL050124 | Homo sapiens mRNA; cDNA | 1.60E−07 |
| | | | DKFZp586E151 (from clone | |
| | | | DKFZp586E151) | |
| 1076 | 3643.L01.gz43_518476 | AJ278429 | Mus musculus partial Prkar1a gene for | 3.00E−06 |
| | | | cAMP-dependent protein kinase | |
| | | | regulatory subunit RIalpha, exons 8-10 | |
| | | | and 3′UTR | |
| 1077 | 3643.N24.gz43_518846 | BC006511 | Homo sapiens, clone IMAGE: 3010441, | 1.00E−05 |
| | | | mRNA | |
| 1078 | 3643.O16.gz43_518719 | AE002303 | Chlamydia muridarum, section 34 of 85 | 1.10E−05 |
| | | | of the complete genome | |
| 1079 | 3643.O18.gz43_518751 | V00248 | Drosophila gene for yolk protein I | 2.00E−06 |
| | | | (vitellogenin) | |
| 1080 | 3643.O21.gz43_518799 | AE000614 | Helicobacter pylori 26695 section 92 of | 1.40E−05 |
| | | | 134 of the complete genome | |
| 1081 | 3643.P13.gz43_518672 | Y17693 | Bungarus multicinctus gene encoding | 2.00E−07 |
| | | | alpha-bungarotoxin, V31 variant | |
| 1082 | 3643.P14.gz43_518688 | AF109352 | Euperipatoides rowelli microsatellite P18 | 8.80E−10 |
| | | | sequence | |
| 1083 | 3646.A07.gz43_518945 | X55137 | H. giganteus type II restriction- | 3.00E−06 |
| | | | modification system HgiBI | |
| 1084 | 3646.A09.gz43_518977 | AF074963 | Rattus norvegicus endothelin-B receptor | 2.10E−07 |
| | | | (EDNRB) gene, partial cds | |
| 1085 | 3646.A12.gz43_519025 | AF176208 | Homo sapiens EcoRI-HindIII fragment | 1.60E−05 |
| | | | upstream of exon 1 of the c-myc gene | |
| 1086 | 3646.A13.gz43_519041 | X89445 | O. chalybea DNA for narB gene and | 4.00E−05 |
| | | | partial ORFs | |
| 1087 | 3646.B20.gz43_519154 | M86514 | Rat proline-rich protein mRNA, 3′ end | 1.60E−05 |
| 1088 | 3646.C06.gz43_518931 | Z71180 | Caenorhabditis elegans cosmid F22E12, | 2.03E−04 |
| | | | complete sequence | |
| 1089 | 3646.C16.gz43_519091 | U73608 | Hepatitis B virus, genome 7648 with G- | 2.30E−05 |
| | | | >A hypermutations | |
| 1090 | 3646.E02.gz43_518869 | U11683 | Trypanoplasma borreli Tt-JH | 8.10E−07 |
| | | | mitochondrion cytochrome c oxidase | |
| | | | subunit 1 (cox1) gene, complete cds | |
| 1091 | 3646.E20.gz43_519157 | AE006216 | Pasteurella multocida PM70 section 183 | 2.30E−05 |
| | | | of 204 of the complete genome | |
| 1092 | 3646.H04.gz43_518904 | AF043740 | Branchiostoma floridae amphioxus Otx | 2.00E−06 |
| | | | transcription factor (Otx) mRNA, | |
| | | | complete cds | |
| 1093 | 3646.H09.gz43_518984 | AP000145 | Homo sapiens genomic DNA, | 2.90E−40 |
| | | | chromosome 21q21.2, LL56-APP region, | |
| | | | clone B2291C14-R44F3, segment 10/10, | |
| | | | complete sequence | |
| 1094 | 3646.H16.gz43_519096 | U22342 | Bacteriophage T270 integrase (int) gene, | 1.00E−07 |
| | | | complete cds | |
| 1095 | 3646.I01.gz43_518857 | X54486 | Human gene for C1-inhibitor | 6.80E−05 |
| 1096 | 3646.J03.gz43_518890 | AB055372 | Macaca fascicularis brain cDNA, | 5.40E−190 |
| | | | clone: QflA-12842 | |
| 1097 | 3646.J22.gz43_519194 | AL133032 | Homo sapiens mRNA; cDNA | 2.00E−06 |
| | | | DKFZp586B0317 (from clone | |
| | | | DKFZp586B0317) | |
| 1098 | 3646.K14.gz43_519067 | AF239178 | Paracoccidioides brasiliensis lon | 4.00E−06 |
| | | | proteinase gene, complete cds; nuclear | |
| | | | gene for mitochondrial product | |
| 1099 | 3646.L17.gz43_519116 | Z58907 | H. sapiens CpG island DNA genomic | 2.50E−70 |
| | | | Mse1 fragment, clone 116a6, forward | |
| | | | read cpg116a6.ft1a | |
| 1100 | 3646.O13.gz43_519055 | AL050391 | Homo sapiens mRNA; cDNA | 5.20E−08 |
| | | | DKFZp586A181 (from clone | |
| | | | DKFZp586A181); partial cds | |
| 1101 | 3646.O16.gz43_519103 | X00331 | Drosophila virilis simple DNA sequence | 5.20E−08 |
| | | | (pDV-161) | |
| 1102 | 3646.P09.gz43_518992 | U04527 | Borrelia burgdorferi 212 DNA gyrase b | 5.00E−06 |
| | | | subunit (gyrB) and ribonuclease P | |
| | | | protein component (rnpA) genes, partial | |
| | | | cds, DnaA protein (dnaA), DNA | |
| | | | polymerase III beta subunit (dnaN), and | |
| | | | ribosomal protein L34 (rpmH) genes, | |
| | | | complete cds | |
| 1103 | 3646.P14.gz43_519072 | AY032863 | Mus musculus chloride-formate | 8.00E−06 |
| | | | exchanger mRNA, complete cds | |
| 1104 | 3646.P17.gz43_519120 | U19569 | Human squamous cell carcinoma antigen | 1.20E−07 |
| | | | (SCCA2) gene, exon 1 | |
| 1105 | 3661.A08.gz43_519483 | AB017511 | Hydra magnipapillata mRNA for PLC- | 1.20E−05 |
| | | | betaH1, complete cds | |
| 1106 | 3661.D17.gz43_519630 | J03488 | Reovirus type 3 L2 gene encoding | 3.00E−06 |
| | | | guanylyltransferase, complete cds | |
| 1107 | 3661.D18.gz43_519646 | AB033024 | Homo sapiens mRNA for KIAA1198 | 1.90E−11 |
| | | | protein, partial cds | |
| 1108 | 3661.E19.gz43_519663 | AB014084 | Homo sapiens genomic DNA, | 6.00E−05 |
| | | | chromosome 6p21.3, HLA class I region, | |
| | | | Cosmid clone: TY7A5, complete | |
| | | | sequence | |
| 1109 | 3661.E23.gz43_519727 | AE001032 | Archaeoglobus fulgidus section 75 of | 5.30E−05 |
| | | | 172 of the complete genome | |
| 1110 | 3661.F14.gz43_519584 | X15063 | Plasmodium falciparum mRNA for | 6.80E−05 |
| | | | major merozoite surface antigen gp195 | |
| 1111 | 3661.G16.gz43_519617 | AF255609 | Homo sapiens high mobility group | 2.70E−07 |
| | | | protein HMG1 gene, exons 1 and 2, | |
| | | | partial cds | |
| 1112 | 3661.G20.gz43_519681 | AK021558 | Homo sapiens cDNA FLJ11496 fis, | 6.40E−09 |
| | | | clone HEMBA1001964 | |
| 1113 | 3661.H11.gz43_519538 | Z30705 | Puumala virus (Evo/15Cg/93) gene for N | 3.90E−07 |
| | | | protein | |
| 1114 | 3661.H24.gz43_519746 | X66979 | X. laevis mRNA XLFLI | 1.60E−05 |
| 1115 | 3661.I22.gz43_519715 | AF029887 | Caenorhabditis elegans UNC-129 (unc- | 5.00E−06 |
| | | | 129) mRNA, complete cds | |
| 1116 | 3661.J15.gz43_519604 | AJ297538 | Homo sapiens partial RARA gene, intron 2 | 4.00E−06 |
| 1117 | 3661.K22.gz43_519717 | AK002100 | Homo sapiens cDNA FLJ11238 fis, | 1.30E−13 |
| | | | clone PLACE1008532 | |
| 1118 | 3661.L19.gz43_519670 | AL589643 | Human DNA sequence from clone | 2.20E−05 |
| | | | RP11-344C1 on chromosome 6, | |
| | | | complete sequence [Homo sapiens] | |
| 1119 | 3661.M03.gz43_519415 | Z57613 | H. sapiens CpG island DNA genomic | 1.20E−08 |
| | | | Mse1 fragment, clone 187a12, forward | |
| | | | read cpg187a12.ft1a | |
| 1120 | 3661.M23.gz43_519735 | X79547 | Equus caballus mitochondrial DNA | 5.80E−05 |
| | | | complete sequence | |
| 1121 | 3661.P22.gz43_519722 | AF055668 | Mus musculus apoptosis-linked gene 4, | 8.00E−06 |
| | | | deltaC form (Alg-4) mRNA, partial cds | |
| 1122 | 3662.A13.gz43_519947 | Z49438 | S. cerevisiae chromosome X reading | 3.00E−06 |
| | | | frame ORF YJL163c | |
| 1123 | 3662.B13.gz43_519948 | AB045237 | Xenopus laevis XRPTPb mRNA for | 7.00E−06 |
| | | | receptor-type protein tyrosine | |
| | | | phosphatase beta.11, complete cds | |
| 1124 | 3662.C10.gz43_519901 | BC007905 | Homo sapiens, Similar to retinal | 1.20E−09 |
| | | | degeneration B beta, clone MGC: 14375 | |
| | | | IMAGE: 4299595, mRNA, complete cds | |
| 1125 | 3662.C15.gz43_519981 | M33864 | Human (cline HGL-3) interstitial | 1.20E−05 |
| | | | retinoid-binding protein 3 (RBP3) gene, | |
| | | | exon 1 | |
| 1126 | 3662.F13.gz43_519952 | AB040935 | Homo sapiens mRNA for KIAA1502 | 1.20E−61 |
| | | | protein, partial cds | |
| 1127 | 3662.H14.gz43_519970 | AB032757 | Mus musculus gad65 gene for glutamate | 8.00E−07 |
| | | | decarboxylase 65, partial cds | |
| 1128 | 3662.H23.gz43_520114 | AK013013 | Mus musculus 10, 11 days embryo | 2.00E−06 |
| | | | cDNA, RIKEN full-length enriched | |
| | | | library, clone: 2810406L04, full insert | |
| | | | sequence | |
| 1129 | 3662.H24.gz43_520130 | D45371 | Human apM1 mRNA for GS3109 (novel | 9.60E−10 |
| | | | adipose specific collagen-like factor), | |
| | | | complete cds | |
| 1130 | 3662.J05.gz43_519828 | M83554 | H. sapiens lymphocyte activation antigen | 1.40E−05 |
| | | | CD30 mRNA, complete cds | |
| 1131 | 3662.J08.gz43_519876 | Z11876 | B. hermsii vmp7 gene encoding Vmp7 | 1.11E−04 |
| | | | outer membrane lipoprotein | |
| 1132 | 3662.J09.gz43_519892 | AB011101 | Homo sapiens mRNA for KIAA0529 | 6.30E−05 |
| | | | protein, partial cds | |
| 1133 | 3662.J16.gz43_520004 | U00484 | Anabaena PCC7120 protein kinase PknA | 2.00E−06 |
| | | | (pknA) gene, complete cds | |
| 1134 | 3662.K03.gz43_519797 | AL390145 | Homo sapiens mRNA; cDNA | 1.40E−05 |
| | | | DKFZp762C115 (from clone | |
| | | | DKFZp762C115) | |
| 1135 | 3662.L05.gz43_519830 | U63635 | Schizosaccharomyces pombe RNA lariat | 5.80E−10 |
| | | | debranching enzyme (Sp-dbr1) gene, | |
| | | | complete cds | |
| 1136 | 3662.N24.gz43_520136 | Z30709 | L. helveticus genes for prolinase and | 3.70E−05 |
| | | | putative ABC transporter | |
| 1137 | 3662.O02.gz43_519785 | AF084460 | Gallus gallus potassium channel Shaker | 6.90E−05 |
| | | | alpha subunit variant cKv1.4(m) | |
| | | | mRNA, complete cds | |
| 1138 | 3662.P03.gz43_519802 | AJ011456 | Schinziella tetragona matK gene | 7.20E−08 |
| | | | (corresponding location in Tobacco: 963-1244) | |
| 1139 | 3663.A09.gz43_520267 | Z69608 | A. rara SSU rRNA gene (partial) | 3.30E−07 |
| 1140 | 3663.C08.gz43_520253 | Z50756 | Caenorhabditis elegans cosmid T08D10, | 7.60E−07 |
| | | | complete sequence | |
| 1141 | 3663.C19.gz43_520429 | Z22672 | H. sapiens cacnl1a3 gene encoding | 2.80E−07 |
| | | | skeletal muscle dhp-receptor alpha 1 | |
| | | | subunit | |
| 1142 | 3663.E04.gz43_520191 | U89318 | Homo sapiens nucleophosmin | 2.60E−07 |
| | | | phosphoprotein (NPM) gene, intron 9, | |
| | | | partial sequence | |
| 1143 | 3663.F15.gz43_520368 | U66073 | Tritrichomonas foetus putative | 9.20E−07 |
| | | | superoxide dismutase 1 (SOD1) gene, | |
| | | | complete cds | |
| 1144 | 3663.F22.gz43_520480 | U36786 | Rattus norvegicus putative pheromone | 7.10E−07 |
| | | | receptor VN7 mRNA, complete cds | |
| 1145 | 3663.G01.gz43_520145 | AK024359 | Homo sapiens cDNA FLJ14297 fis, | 9.50E−36 |
| | | | clone PLACE1008941 | |
| 1146 | 3663.G08.gz43_520257 | L19339 | Molgula oculata zinc finger protein | 5.20E−07 |
| | | | (manx) mRNA, complete cds | |
| 1147 | 3663.H20.gz43_520450 | X61307 | Staphylococcus aureus spa gene for | 5.00E−06 |
| | | | protein A | |
| 1148 | 3663.J06.gz43_520228 | AE007916 | Agrobacterium tumefaciens strain C58 | 2.02E−04 |
| | | | plasmid AT, section 44 of 50 of the | |
| | | | complete sequence | |
| 1149 | 3663.J16.gz43_520388 | U38181 | Leuconostoc mesenteroides | 3.90E−07 |
| | | | dextransucrase gene, complete cds | |
| 1150 | 3663.K02.gz43_520165 | X68339 | Mycoplasma-like organism (substrain | 5.00E−06 |
| | | | ASHY) DNA for 16S rRNA | |
| 1151 | 3663.K13.gz43_520341 | AF155221 | Mus musculus matrix metalloproteinase | 2.00E−06 |
| | | | 19 (Mmp19) mRNA, complete cds | |
| 1152 | 3663.L18.gz43_520422 | AB031056 | Solobacterium moorei gene for 16S | 1.00E−06 |
| | | | rRNA, isolate: RCA59-74 | |
| 1153 | 3663.L24.gz43_520518 | D10445 | Mouse mRNA for protein C, complete | 6.00E−06 |
| | | | cds | |
| 1154 | 3663.M24.gz43_520519 | AE001196 | Treponema pallidum section 12 of 87 of | 5.20E−05 |
| | | | the complete genome | |
| 1155 | 3663.N09.gz43_520280 | AF081788 | Homo sapiens putative spliceosome | 4.00E−20 |
| | | | associated protein mRNA, complete cds | |
| 1156 | 3663.N10.gz43_520296 | X62423 | P. falciparum pol delta gene for DNA | 4.00E−06 |
| | | | polymerase delta | |
| 1157 | 3663.N12.gz43_520328 | AF178079 | Zygosaccharomyces rouxii ketoreductase | 5.00E−06 |
| | | | (krd) mRNA, complete cds | |
| 1158 | 3663.N16.gz43_520392 | U41060 | Homo sapiens estrogen regulated LIV-1 | 2.00E−06 |
| | | | protein (LIV-1) mRNA, complete cds | |
| 1159 | 3663.O07.gz43_520249 | D00442 | Grapevine fanleaf virus satellite RNA | 1.50E−08 |
| | | | (RNA3), complete cds | |
| 1160 | 3663.O09.gz43_520281 | AK002141 | Homo sapiens cDNA FLJ11279 fis, | 5.30E−10 |
| | | | clone PLACE1009444, highly similar to | |
| | | | PHOSPHATIDYLINOSITOL 4- | |
| | | | KINASE ALPHA (EC 2.7.1.67) | |
| 1161 | 3664.A11.gz43_520683 | U67525 | Methanococcus jannaschii section 67 of | 4.00E−06 |
| | | | 150 of the complete genome | |
| 1162 | 3664.C21.gz43_520845 | AF064773 | Staphylococcus aureus extracellular | 1.30E−07 |
| | | | enterotoxin type G precursor (SEG) | |
| | | | gene, complete cds | |
| 1163 | 3664.D06.gz43_520606 | AF178079 | Zygosaccharomyces rouxii ketoreductase | 5.00E−06 |
| | | | (krd) mRNA, complete cds | |
| 1164 | 3664.D12.gz43_520702 | U10519 | Human DNA polymerase beta gene, | 2.00E−07 |
| | | | exon 5 | |
| 1165 | 3664.D17.gz43_520782 | AK027226 | Homo sapiens cDNA: FLJ23573 fis, | 4.90E−07 |
| | | | clone LNG12520 | |
| 1166 | 3664.E18.gz43_520799 | AF317204 | Mus musculus C-type lectin superfamily | 3.20E−05 |
| | | | 1 gene, complete cds | |
| 1167 | 3664.E23.gz43_520879 | AB050903 | Mus musculus mRNA for a4 subunit | 3.00E−06 |
| | | | isoform, complete cds | |
| 1168 | 3664.E24.gz43_520895 | Z92793 | Caenorhabditis elegans cosmid H15M21, | 1.20E−05 |
| | | | complete sequence | |
| 1169 | 3664.G12.gz43_520705 | AF211482 | Dictyostelium discoideum SdhA (sdhA) | 2.30E−09 |
| | | | gene, complete cds | |
| 1170 | 3664.G20.gz43_520833 | M14450 | Rat thyrotropin (TSH) beta-subunit gene, | 4.00E−06 |
| | | | exons 2 and 3 | |
| 1171 | 3664.H15.gz43_520754 | Y11270 | E. histolytica INO1 gene | 2.00E−06 |
| 1172 | 3664.H22.gz43_520866 | X97773 | B. taurus mRNA for mitochondrial | 1.20E−05 |
| | | | tricarboxylate carrier protein | |
| 1173 | 3664.J12.gz43_520708 | M58150 | Bovine lactoperoxidase (LPO) mRNA, | 3.20E−05 |
| | | | complete cds | |
| 1174 | 3664.J23.gz43_520884 | U67463 | Methanococcus jannaschii section 5 of | 3.00E−06 |
| | | | 150 of the complete genome | |
| 1175 | 3664.K16.gz43_520773 | Z83118 | Caenorhabditis elegans cosmid M04D5, | 2.70E−07 |
| | | | complete sequence | |
| 1176 | 3664.K19.gz43_520821 | U36927 | Plasmodium yoelii rhoptry protein gene, | 3.00E−05 |
| | | | complete cds | |
| 1177 | 3664.L21.gz43_520854 | AF057695 | Haemophilus ducreyi strain 35000 | 2.15E−04 |
| | | | putative phosphomannomutase (pmm) | |
| | | | gene, partial cds; large supernatant | |
| | | | protein 1 (lspA1) gene, complete cds; | |
| | | | and putative GMP synthase (guaA) gene, | |
| | | | partial cds | |
| 1178 | 3664.O22.gz43_520873 | U43574 | Hydra vulgaris nucleoporin p62 gene, | 7.00E−06 |
| | | | complete cds | |
| 1179 | 3664.P12.gz43_520714 | AF030883 | Mus musculus tRNA-His gene, complete | 9.00E−06 |
| | | | sequence; platelet-activating factor | |
| | | | acetylhydrolase Ib alpha subunit (Pafaha- | |
| | | | psl) pseudogene, complete sequence; | |
| | | | and tRNA-Glu gene, complete sequence | |
| 1180 | 3664.P18.gz43_520810 | Z47735 | H. sapiens NFKB1 gene, exons 11 & 12 | 1.32E−04 |
| 1181 | 3665.A23.gz43_521259 | X66979 | X. laevis mRNA XLFLI | 1.60E−05 |
| 1182 | 3665.B01.gz43_520908 | M90058 | Human serglycin gene, exons 1, 2, and 3 | 4.00E−06 |
| 1183 | 3665.B12.gz43_521084 | AK020877 | Mus musculus adult retina cDNA, | 7.10E−07 |
| | | | RIKEN full-length enriched library, | |
| | | | clone: A930019H03, full insert sequence | |
| 1184 | 3665.E11.gz43_521071 | AB024030 | Arabidopsis thaliana genomic DNA, | 9.00E−06 |
| | | | chromosome 5, TAC clone: K5A21 | |
| 1185 | 3665.E20.gz43_521215 | X76584 | H. sapiens simple DNA sequence region | 6.80E−08 |
| | | | clone wg1h1 | |
| 1186 | 3665.H20.gz43_521218 | X95301 | D. rerio mRNA for HER-5 protein | 9.50E−07 |
| 1187 | 3665.K01.gz43_520917 | X04653 | Mouse mRNA for Ly-6 alloantigen (Ly- | 1.30E−05 |
| | | | 6E.1) | |
| 1188 | 3665.M01.gz43_520919 | AF098352 | Wiseana copularis haplotype southern | 5.80E−07 |
| | | | cytochrome oxidase subunit I and | |
| | | | cytochrome oxidase subunit II genes, | |
| | | | partial cds; mitochondrial genes for | |
| | | | mitochondrial products | |
| 1189 | 3665.M21.gz43_521239 | AF257480 | Rana temporaria microsatellite SB80 | 3.30E−09 |
| | | | sequence | |
| 1190 | 3665.M23.gz43_521271 | Y10623 | C. pallidivittatus globin gene cluster E | 1.10E−05 |
| 1191 | 3665.N24.gz43_521288 | X95301 | D. rerio mRNA for HER-5 protein | 1.00E−06 |
| 1192 | 3665.O06.gz43_521001 | AE007033 | Mycobacterium tuberculosis CDC1551, | 7.40E−05 |
| | | | section 119 of 280 of the complete | |
| | | | genome | |
| 1193 | 3665.O14.gz43_521129 | AB033094 | Homo sapiens mRNA for KIAA1268 | 2.10E−08 |
| | | | protein, partial cds | |
| 1194 | 3665.O15.gz43_521145 | AK004557 | Mus musculus adult male lung cDNA, | 1.20E−05 |
| | | | RIKEN full-length enriched library, | |
| | | | clone: 1200003C23, full insert sequence | |
| 1195 | 3665.O19.gz43_521209 | AY036905 | Trichoderma atroviride protein GTPase | 2.10E−08 |
| | | | Tga1 (tga1) gene, complete cds | |
| 1196 | 3665.O21.gz43_521241 | U89293 | Homo sapiens MSH4 (HMSH4) mRNA, | 1.20E−39 |
| | | | complete cds | |
| 1197 | 3665.O23.gz43_521273 | X00048 | Herpes simplex virus (HSV) type 2 | 6.00E−06 |
| | | | transforming region mtr-2 (map | |
| | | | coordinates 0.580-0.625) | |
| 1198 | 3665.P13.gz43_521114 | Z48796 | H. sapiens Ski-W mRNA for helicase | 1.70E−05 |
| 1199 | 3666.A07.gz43_521387 | AK005546 | Mus musculus adult female placenta | 1.20E−07 |
| | | | cDNA, RIKEN full-length enriched | |
| | | | library, clone: 1600027G01, full insert | |
| | | | sequence | |
| 1200 | 3666.A19.gz43_521579 | AB011101 | Homo sapiens mRNA for KIAA0529 | 5.80E−05 |
| | | | protein, partial cds | |
| 1201 | 3666.A24.gz43_521659 | AL050208 | Homo sapiens mRNA; cDNA | 2.90E−07 |
| | | | DKFZp586F2323 (from clone | |
| | | | DKFZp586F2323) | |
| 1202 | 3666.B11.gz43_521452 | X06932 | Petunia hsp70 gene | 3.00E−06 |
| 1203 | 3666.C18.gz43_521565 | Z22672 | H. sapiens cacnl1a3 gene encoding | 2.80E−07 |
| | | | skeletal muscle dhp-receptor alpha 1 | |
| | | | subunit | |
| 1204 | 3666.D02.gz43_521310 | AJ297538 | Homo sapiens partial RARA gene, intron 2 | 4.00E−06 |
| 1205 | 3666.D11.gz43_521454 | AF057695 | Haemophilus ducreyi strain 35000 | 2.43E−04 |
| | | | putative phosphomannomutase (pmm) | |
| | | | gene, partial cds; large supernatant | |
| | | | protein 1 (lspA1) gene, complete cds; | |
| | | | and putative GMP synthase (guaA) gene, | |
| | | | partial cds | |
| 1206 | 3666.D15.gz43_521518 | Z66194 | H. sapiens CpG island DNA genomic | 1.70E−66 |
| | | | Mse1 fragment, clone 80b12, forward | |
| | | | read cpg80b12.ft1b | |
| 1207 | 3666.D16.gz43_521534 | Z66194 | H. sapiens CpG island DNA genomic | 2.10E−37 |
| | | | Mse1 fragment, clone 80b12, forward | |
| | | | read cpg80b12.ft1b | |
| 1208 | 3666.F22.gz43_521632 | U97062 | Staphylococcus aureus NCTC 8325 | 1.20E−08 |
| | | | SecA (secA) gene, complete cds | |
| 1209 | 3666.G12.gz43_521473 | J03901 | Maize pyruvate, orthophosphate dikinase | 1.72E−04 |
| | | | mRNA, complete cds | |
| 1210 | 3666.I12.gz43_521475 | AJ225102 | Pinus lambertiana chloroplast DNA | 6.40E−10 |
| | | | containing a SSR Black Hills (Oregon) | |
| 1211 | 3666.L01.gz43_521302 | M86227 | Staphylococcus aureus DNA gyrase B | 5.00E−06 |
| | | | subunit (gyrB) RecF homologue (recF) | |
| | | | and DNA gyrase A subunit (gyrA) gene, | |
| | | | complete cds | |
| 1212 | 3666.L06.gz43_521382 | AF224725 | Trichosurus vulpecula retrovirus TvERV | 3.30E−08 |
| | | | (type D) gag polyprotein (gag), protease | |
| | | | (pro), and pol polyprotein (pol) genes, | |
| | | | complete cds | |
| 1213 | 3666.L11.gz43_521462 | AF147081 | Homo sapiens gamma-glutamyl | 3.30E−05 |
| | | | hydrolase gene, exons 1 and 2 | |
| 1214 | 3666.L23.gz43_521654 | AK020701 | Mus musculus 6 days neonate skin | 2.20E−07 |
| | | | cDNA, RIKEN full-length enriched | |
| | | | library, clone: A030009B12, full insert | |
| | | | sequence | |
| 1215 | 3666.M16.gz43_521543 | AF158179 | Drosophila melanogaster strain Canton-S | 4.40E−07 |
| | | | Chiffon-2 (chiffon) mRNA, alternative | |
| | | | splice form 2, complete cds | |
| 1216 | 3666.N06.gz43_521384 | Z48796 | H. sapiens Ski-W mRNA for helicase | 1.70E−05 |
| 1217 | 3667.A15.gz43_524557 | AF005903 | Monodelphis domestica GTP-binding | 7.80E−08 |
| | | | protein homolog mRNA, partial cds | |
| 1218 | 3754.A08.gz43_532949 | AF091502 | Lactobacillus reuteri autoaggregation- | 1.00E−06 |
| | | | mediating protein (aggH) gene, complete | |
| | | | cds | |
| 1219 | 3754.A13.gz43_533029 | U02695 | Protomelas similis clone PsiI 32 SATA | 7.60E−07 |
| | | | satellite DNA sequence | |
| 1220 | 3754.A16.gz43_533077 | AE006577 | Streptococcus pyogenes M1 GAS strain | 9.00E−06 |
| | | | SF370, section 106 of 167 of the | |
| | | | complete genome | |
| 1221 | 3754.B04.gz43_532886 | S83995 | Pst1 fragment [Chlamydia pneumoniae, | 2.00E−06 |
| | | | Genomic, 474 nt] | |
| 1222 | 3754.B05.gz43_532902 | AY008833 | Staphylococcus aureus tcaR-tcaA-tcaB | 5.00E−06 |
| | | | operon, complete sequences | |
| 1223 | 3754.B07.gz43_532934 | AF270216 | Staphylococcus epidermidis strain SR1 | 9.50E−07 |
| | | | clone step.1054h11 genomic sequence | |
| 1224 | 3754.B08.gz43_532950 | AK007308 | Mus musculus adult male testis cDNA, | 7.00E−06 |
| | | | RIKEN full-length enriched library, | |
| | | | clone: 1700128E15, full insert sequence | |
| 1225 | 3754.B10.gz43_532982 | AE002807 | Drosophila melanogaster genomic | 5.40E−05 |
| | | | scaffold 142000013385251, complete | |
| | | | sequence | |
| 1226 | 3754.C22.gz43_533175 | D30612 | Homo sapiens mRNA for repressor | 4.00E−06 |
| | | | protein, partial cds | |
| 1227 | 3754.D19.gz43_533128 | L12043 | Plasmodium falciparum unidentified | 3.00E−06 |
| | | | mRNA sequence | |
| 1228 | 3754.E12.gz43_533017 | AB062933 | Macaca fascicularis brain cDNA | 3.60E−07 |
| | | | clone: QccE-22249, full insert sequence | |
| 1229 | 3754.E20.gz43_533145 | AL138746 | Human DNA sequence from clone RP3- | 8.30E−10 |
| | | | 389B13 on chromosome Xq26.2-27.1, | |
| | | | complete sequence [Homo sapiens] | |
| 1230 | 3754.F01.gz43_532842 | AF086820 | Drosophila melanogaster paired-like | 8.00E−06 |
| | | | homeodomain protein UNC-4 (unc-4) | |
| | | | mRNA, complete cds | |
| 1231 | 3754.F08.gz43_532954 | S66402 | vascular AT1a angiotensin receptor | 3.10E−05 |
| | | | {exon 1, promoter} [rats, Sprague- | |
| | | | Dawley, Genomic, 3477 nt] | |
| 1232 | 3754.F11.gz43_533002 | X57377 | Mouse dilute myosin heavy chain gene | 2.10E−05 |
| | | | for novel heavy chain with unique C- | |
| | | | terminal region | |
| 1233 | 3754.F15.gz43_533066 | AJ245620 | Homo sapiens CTL1 gene | 2.50E−12 |
| 1234 | 3754.F20.gz43_533146 | AE002426 | Neisseria meningitidis serogroup B strain | 3.70E−05 |
| | | | MC58 section 68 of 206 of the complete | |
| | | | genome | |
| 1235 | 3754.G03.gz43_532875 | AF002166 | Xenopus laevis Ig mu heavy chain | 1.20E−07 |
| | | | switch region sequence | |
| 1236 | 3754.G08.gz43_532955 | X71020 | N. tabacum Npg1 gene for | 6.80E−07 |
| | | | polygalacturonase | |
| 1237 | 3754.G18.gz43_533115 | AF126531 | Homo sapiens putative DNA-directed | 1.10E−13 |
| | | | RNA polymerase III C11 subunit gene, | |
| | | | complete cds | |
| 1238 | 3754.H08.gz43_532956 | L20127 | Rochalimaea henselae antigen (htrA) | 4.60E−07 |
| | | | gene, complete cds | |
| 1239 | 3754.I01.gz43_532845 | AK022138 | Homo sapiens cDNA FLJ12076 fis, | 3.90E−14 |
| | | | clone HEMBB1002442, weakly similar | |
| | | | to LIN-10 PROTEIN | |
| 1240 | 3754.I03.gz43_532877 | AF016653 | Caenorhabditis elegans cosmid C41D7, | 2.00E−06 |
| | | | complete sequence | |
| 1241 | 3754.J01.gz43_532846 | U97408 | Caenorhabditis elegans cosmid F48A9 | 4.00E−06 |
| 1242 | 3754.J05.gz43_532910 | Z35484 | Thermoanaerobacter sp. ATCC53627 | 4.00E−06 |
| | | | cgtA gene | |
| 1243 | 3754.J10.gz43_532990 | D17094 | Human HepG2 partial cDNA, clone | 5.10E−11 |
| | | | hmd5h04m5 | |
| 1244 | 3754.J12.gz43_533022 | Z56695 | H. sapiens CpG island DNA genomic | 1.00E−06 |
| | | | Mse1 fragment, clone 136d4, reverse | |
| | | | read cpg136d4.rt1a | |
| 1245 | 3754.J24.gz43_533214 | Y12855 | Homo sapiens P2X7 gene, exon 12 and | 2.50E−05 |
| | | | 13 | |
| 1246 | 3754.K14.gz43_533055 | L79913 | Xenopus laevis rds/peripherin (rds35) | 5.00E−06 |
| | | | mRNA, complete cds | |
| 1247 | 3754.K17.gz43_533103 | AE006251 | Lactococcus lactis subsp. lactis IL1403 | 9.00E−06 |
| | | | section 13 of 218 of the complete | |
| | | | genome | |
| 1248 | 3754.K20.gz43_533151 | AB047880 | Macaca fascicularis brain cDNA, | 1.00E−06 |
| | | | clone: QnpA-14303 | |
| 1249 | 3754.M08.gz43_532961 | X58467 | Human CYP2D7AP pseudogene for | 4.30E−11 |
| | | | cytochrome P450 2D6 | |
| 1250 | 3754.N16.gz43_533090 | U33116 | Saccharomyces cerevisiae high copy | 1.80E−07 |
| | | | DNA polymerase suppressor alpha | |
| | | | mutation gene (PSP2), complete cds | |
| 1251 | 3754.N19.gz43_533138 | AK025312 | Homo sapiens cDNA: FLJ21659 fis, | 1.40E−07 |
| | | | clone COL08743 | |
| 1252 | 3754.N22.gz43_533186 | AF081828 | Ixodes hexagonus mitochondrial DNA, | 4.00E−06 |
| | | | complete genome | |
| 1253 | 3754.O18.gz43_533123 | Z73229 | S. cerevisiae chromosome XII reading | 3.00E−06 |
| | | | frame ORF YLR057w | |
| 1254 | 3754.O23.gz43_533203 | AE006900 | Sulfolobus solfataricus section 259 of | 1.10E−05 |
| | | | 272 of the complete genome | |
| 1255 | 3754.P13.gz43_533044 | AF220217 | Homo sapiens rsec15-like protein | 1.80E−10 |
| | | | mRNA, partial cds | |
| 1256 | 3754.P17.gz43_533108 | AJ250862 | Bacillus sp. HIL-Y85/54728 mersacidin | 1.20E−05 |
| | | | biosynthesis gene cluster (mrsK2, | |
| | | | mrsR2, mrsF, mrsG, mrsE, mrsA, | |
| | | | mrsR1, mrsD, mrsM and mrsT genes) | |
| 1257 | 3756.A02.gz43_533237 | AF285594 | Homo sapiens testis protein TEX11 | 1.10E−05 |
| | | | (TEX11) mRNA, complete cds | |
| 1258 | 3756.A11.gz43_533381 | U43148 | Human patched homolog (PTC) mRNA, | 4.00E−06 |
| | | | complete cds | |
| 1259 | 3756.A13.gz43_533413 | U56861 | Nicotiana plumbaginifolia intergenic | 1.00E−06 |
| | | | region between lhcb1*1 and lhcb1*2 | |
| | | | genes | |
| 1260 | 3756.B03.gz43_533254 | AF101735 | Pan troglodytes isolate PTOR3A5P | 5.70E−08 |
| | | | olfactory receptor pseudogene, complete | |
| | | | sequence | |
| 1261 | 3756.B04.gz43_533270 | Z82038 | C. thermosaccharolyticum etfB, etfA, | 1.00E−06 |
| | | | hbd, thlA and actA genes | |
| 1262 | 3756.B15.gz43_533446 | M96151 | Mus musculus apolipoprotein B gene | 1.13E−04 |
| | | | sequence | |
| 1263 | 3756.B21.gz43_533542 | Z92793 | Caenorhabditis elegans cosmid H15M21, | 1.30E−05 |
| | | | complete sequence | |
| 1264 | 3756.B22.gz43_533558 | U43542 | Nicotiana tabacum diphenol oxidase | 2.00E−06 |
| | | | mRNA, complete cds | |
| 1265 | 3756.C06.gz43_533303 | AB022085 | Mus musculus Cctz-2 gene for | 7.00E−05 |
| | | | chaperonin containing TCP-1 zeta-2 | |
| | | | subunit, exon 5, 6, 7, 8, 9, 10 | |
| 1266 | 3756.C16.gz43_533463 | AF143236 | Homo sapiens apoptosis related protein | 5.00E−06 |
| | | | APR-2 mRNA, complete cds | |
| 1267 | 3756.D08.gz43_533336 | AB049544 | Porcine enterovirus 10 gene for RNA- | 7.20E−07 |
| | | | dependent RNA polymerase, partial cds | |
| 1268 | 3756.D18.gz43_533496 | X53658 | E. coli DNA fragment | 7.60E−08 |
| 1269 | 3756.D24.gz43_533592 | X96861 | H. virescens mRNA for pheromone | 2.40E−07 |
| | | | binding protein | |
| 1270 | 3756.E01.gz43_533225 | AF202892 | Mus musculus Kif21a (Kif21a) mRNA, | 4.00E−06 |
| | | | complete cds | |
| 1271 | 3756.E06.gz43_533305 | AF139374 | Homo sapiens DIR1 protein (DIR1) | 8.00E−06 |
| | | | gene, complete cds | |
| 1272 | 3756.E12.gz43_533401 | AF238884 | Botrytis virus F, complete genome | 8.00E−06 |
| 1273 | 3756.E22.gz43_533561 | U78866 | Arabidopsis thaliana putative arginine- | 5.00E−06 |
| | | | aspartate-rich RNA binding protein | |
| | | | (gene1500), (gene1000), and (gene400) | |
| | | | genes, complete cds | |
| 1274 | 3756.F11.gz43_533386 | D50091 | Drosophila ezoana G-3-P dehydrogenase | 2.00E−06 |
| | | | (alphaGpdh) gene, exon1-8, complete | |
| | | | cds | |
| 1275 | 3756.F16.gz43_533466 | AJ233973 | Gallus gallus microsatellite DNA | 4.20E−07 |
| | | | GCT028 (CA) repeat | |
| 1276 | 3756.G07.gz43_533323 | AE000708 | Aquifex aeolicus section 40 of 109 of the | 6.00E−05 |
| | | | complete genome | |
| 1277 | 3756.G12.gz43_533403 | M84731 | Pseudomonas sp. 5-substituted hydantoin | 1.20E−05 |
| | | | racemase (hyuE) gene, complete cds | |
| 1278 | 3756.G14.gz43_533435 | AL116458 | Botrytis cinerea strain T4 cDNA library | 6.70E−07 |
| | | | under conditions of nitrogen deprivation | |
| 1279 | 3756.I03.gz43_533261 | U67550 | Methanococcus jannaschii section 92 of | 2.30E−05 |
| | | | 150 of the complete genome | |
| 1280 | 3756.J05.gz43_533294 | U11292 | Human Ki nuclear autoantigen mRNA, | 7.70E−07 |
| | | | complete cds | |
| 1281 | 3756.K03.gz43_533263 | AF073484 | Homo sapiens MHC class I-related | 8.00E−06 |
| | | | protein MR1 precursor (MR1) gene, | |
| | | | signal peptide | |
| 1282 | 3756.K07.gz43_533327 | M37499 | Human methylmalonyl CoA mutase | 2.00E−06 |
| | | | (MUT) gene, exon 2 | |
| 1283 | 3756.K15.gz43_533455 | AF248820 | Maoricicada campbelli isolate TB-MC- | 7.30E−07 |
| | | | 016 tRNA-Asp gene, complete sequence; | |
| | | | ATPase subunit 8 gene, complete cds; | |
| | | | and ATPase subunit 6 gene, partial cds; | |
| | | | mitochondrial genes for mitochondrial | |
| | | | products | |
| 1284 | 3756.K18.gz43_533503 | M36300 | S. cerevisiae glutamine amidotransferase | 2.30E−05 |
| | | | (TRP3) gene, 3′ end | |
| 1285 | 3756.K20.gz43_533535 | AY022480 | Oryza sativa microsatellite MRG4805 | 2.00E−10 |
| | | | containing (AGG)X8, genomic sequence | |
| 1286 | 3756.L02.gz43_533248 | X03833 | Human gene for interleukin 1 alpha (IL-1 | 2.80E−12 |
| | | | alpha) | |
| 1287 | 3756.L03.gz43_533264 | AF244246 | Dysdera sp. MC cytochrome c oxidase I | 2.70E−07 |
| | | | (COI) gene, partial cds; mitochondrial | |
| | | | gene for mitochondrial product | |
| 1288 | 3756.L19.gz43_533520 | AJ002732 | Schizosaccharomyces pombe mRNA for | 2.00E−06 |
| | | | ribosomal protein 114 | |
| 1289 | 3756.M06.gz43_533313 | AK002951 | Mus musculus adult male brain cDNA, | 3.60E−07 |
| | | | RIKEN full-length enriched library, | |
| | | | clone: 0710001E20, full insert sequence | |
| 1290 | 3756.M07.gz43_533329 | AF057708 | Populus balsamifera subsp. trichocarpa | 2.60E−07 |
| | | | PTD protein (PTD) gene, complete cds | |
| 1291 | 3756.M20.gz43_533537 | Z35821 | S. cerevisiae chromosome II reading | 2.00E−06 |
| | | | frame ORF YBL060w | |
| 1292 | 3756.N18.gz43_533506 | AL591667 | Human DNA sequence from clone | 6.10E−05 |
| | | | RP11-389N9 on chromosome 6, | |
| | | | complete sequence [Homo sapiens] | |
| 1293 | 3756.N21.gz43_533554 | AK026258 | Homo sapiens cDNA: FLJ22605 fis, | 2.00E−06 |
| | | | clone HSI04743 | |
| 1294 | 3756.O03.gz43_533267 | U61347 | Leiophyllum buxifolium ribosomal | 4.20E−07 |
| | | | maturase (matK) gene, chloroplast gene | |
| | | | encoding chloroplast protein, complete | |
| | | | cds | |
| 1295 | 3756.O07.gz43_533331 | AF177871 | Drosophila melanogaster small GTPase | 5.70E−07 |
| | | | RHO1 (Rho1) gene, alternatively spliced | |
| | | | products and complete cds | |
| 1296 | 3756.O08.gz43_533347 | M60705 | Homo sapiens type I DNA | 6.00E−06 |
| | | | topoisomerase gene, exons 19 and 20 | |
| 1297 | 3756.P08.gz43_533348 | M60705 | Homo sapiens type I DNA | 1.00E−05 |
| | | | topoisomerase gene, exons 19 and 20 | |
| 1298 | 3759.C01.gz43_533607 | X71874 | H. sapiens genes for proteasome-like | 4.00E−06 |
| | | | subunit (MECL-1), chymotrypsin-like | |
| | | | protease (CTRL-1) and protein serine | |
| | | | kinase (PSK-H1) last exon | |
| 1299 | 3759.D15.gz43_533832 | AL356790 | Human DNA sequence from clone | 1.10E−07 |
| | | | RP11-238J15 on chromosome 20 | |
| | | | Contains ESTs and GSSs. Contains part | |
| | | | of the TOM gene for a putative | |
| | | | mitochondrial outer membrane protein | |
| | | | import receptor similar to yeast pre- | |
| | | | mRNA splicing factors Prp1/Zer1 and | |
| | | | Prp6, complete> | |
| 1300 | 3759.H08.gz43_533724 | M31684 | D. melanogaster cytoskeleton-like | 2.00E−06 |
| | | | bicaudalD protein (BicD) mRNA, | |
| | | | complete cds | |
| 1301 | 3759.H15.gz43_533836 | AB046001 | Macaca fascicularis brain cDNA, | 2.60E−07 |
| | | | clone: QccE-12738 | |
| 1302 | 3759.H17.gz43_533868 | AE000706 | Aquifex aeolicus section 38 of 109 of the | 1.30E−05 |
| | | | complete genome | |
| 1303 | 3759.H23.gz43_533964 | AK027088 | Homo sapiens cDNA: FLJ23435 fis, | 6.20E−34 |
| | | | clone HRC12631 | |
| 1304 | 3759.I05.gz43_533677 | AF056433 | Homo sapiens clone FBD3 Cri-du-chat | 1.70E−07 |
| | | | critical region mRNA | |
| 1305 | 3759.I19.gz43_533901 | Z69666 | Human DNA sequence from cosmid | 2.06E−04 |
| | | | 24F8 from a contig from the tip of the | |
| | | | short arm of chromosome 16, spanning | |
| | | | 2 Mb of 16p13.3. Contains ESTs, repeat | |
| | | | polymorphism and CpG island | |
| 1306 | 3759.K05.gz43_533679 | L01432 | Soybean calmodulin (SCaM-3) mRNA, | 4.10E−08 |
| | | | complete cds | |
| 1307 | 3759.K17.gz43_533871 | Z33340 | M. capricolum DNA for CONTIG | 4.00E−06 |
| | | | MC456 | |
| 1308 | 3759.L02.gz43_533632 | U26736 | Caenorhabditis elegans stomatin-like | 3.70E−05 |
| | | | protein MEC-2 (mec-2) gene, complete | |
| | | | cds | |
| 1309 | 3759.L09.gz43_533744 | M11180 | Transposon Tn917 (complete), | 1.50E−07 |
| | | | macrolide-lincosamide-streptogramin-B | |
| | | | (MLS) resistance, complete cds | |
| 1310 | 3759.L10.gz43_533760 | AF117022 | Solaria atropurpurea trnL gene, partial | 4.40E−07 |
| | | | sequence; chloroplast gene for | |
| | | | chloroplast product | |
| 1311 | 3759.L15.gz43_533840 | U22657 | Mus musculus genomic locus related to | 1.60E−05 |
| | | | cellular morphology | |
| 1312 | 3759.L24.gz43_533984 | AK022990 | Homo sapiens cDNA FLJ12928 fis, | 7.60E−10 |
| | | | clone NT2RP2004767 | |
| 1313 | 3759.M19.gz43_533905 | M96324 | Lycopersicon esculentum Ca2+-ATPase | 2.50E−05 |
| | | | gene, complete cds | |
| 1314 | 3759.N08.gz43_533730 | AK005546 | Mus musculus adult female placenta | 1.30E−07 |
| | | | cDNA, RIKEN full-length enriched | |
| | | | library, clone: 1600027G01, full insert | |
| | | | sequence | |
| 1315 | 3759.N16.gz43_533858 | AB014079 | Homo sapiens genomic DNA, | 3.80E−12 |
| | | | chromosome 6p21.3, HLA class I region, | |
| | | | Cosmid clone: TY1E11, complete | |
| | | | sequence | |
| 1316 | 3759.N23.gz43_533970 | AK018377 | Mus musculus 16 days embryo lung | 5.70E−07 |
| | | | cDNA, RIKEN full-length enriched | |
| | | | library, clone: 8430403M08, full insert | |
| | | | sequence | |
| 1317 | 3759.O16.gz43_533859 | AE000918 | Methanobacterium thermoautotrophicum | 1.40E−05 |
| | | | from bases 1444576 to 1460617 (section | |
| | | | 124 of 148) of the complete genome | |
| 1318 | 3759.P03.gz43_533652 | L06066 | Saccharomyces cerevisiae PET117 | 5.90E−07 |
| | | | polypeptide (PET117) gene, complete | |
| | | | cds | |
| 1319 | 3759.P13.gz43_533812 | X89414 | A. thaliana DNA for pyrroline-5- | 5.00E−06 |
| | | | carboxylase synthetase gene | |
| 1320 | 3759.P15.gz43_533844 | X66979 | X. laevis mRNA XLFLI | 1.50E−05 |
| 1321 | 3759.P17.gz43_533876 | AF039313 | Moraxella catarrhalis strain LES-1 | 2.00E−06 |
| | | | transferrin binding protein B (tbpB) | |
| | | | gene, complete cds | |
| 1322 | 3762.A09.gz43_534117 | AE000496 | Escherichia coli K12 MG1655 section | 1.63E−04 |
| | | | 386 of 400 of the complete genome | |
| 1323 | 3762.A16.gz43_534229 | X98371 | D. subobscura sex-lethal gene | 7.00E−06 |
| 1324 | 3762.A19.gz43_534277 | U95019 | Human voltage-dependent calcium | 6.10E−07 |
| | | | channel beta-2c subunit mRNA, | |
| | | | complete cds | |
| 1325 | 3762.A20.gz43_534293 | M10014 | Homo sapiens map 4q28 fibrinogen | 8.00E−06 |
| | | | (FGG) gene, alternative splice products, | |
| | | | complete cds | |
| 1326 | 3762.B05.gz43_534054 | J05614 | Human proliferating cell nuclear antigen | 1.40E−05 |
| | | | (PCNA) gene, promoter region | |
| 1327 | 3762.B15.gz43_534214 | AJ297559 | Homo sapiens partial PIK3CB gene for | 2.50E−05 |
| | | | phosphatidylinositol 3-kinase catalytic | |
| | | | subunit p110beta, exons 15-17 | |
| 1328 | 3762.C20.gz43_534295 | M58580 | Rabbit angiotensin-converting enzyme | 3.10E−05 |
| | | | (ACE) gene, 5′ end | |
| 1329 | 3762.C23.gz43_534343 | L27146 | Human neurofibromatosis 2 (NF2) gene, | 1.00E−06 |
| | | | exon 16 | |
| 1330 | 3762.D03.gz43_534024 | U51305 | Triticum aestivum alpha-gliadin storage | 1.40E−05 |
| | | | protein pseudogene, complete cds | |
| 1331 | 3762.D04.gz43_534040 | AF263274 | Chionodraco rastrospinosus isolate Cra7 | 3.50E−07 |
| | | | alpha tubulin mRNA, complete cds | |
| 1332 | 3762.D18.gz43_534264 | M94764 | Glycine max cv. Dare nodulin 26 gene | 2.50E−05 |
| | | | fragment | |
| 1333 | 3762.D19.gz43_534280 | AE001446 | Helicobacter pylori, strain J99 section 7 | 3.30E−05 |
| | | | of 132 of the complete genome | |
| 1334 | 3762.D22.gz43_534328 | M73962 | Bovine pregnancy-associated | 4.00E−06 |
| | | | glycoprotein 1 mRNA, complete cds | |
| 1335 | 3762.E01.gz43_533993 | X63746 | S. cerevisiae rpc34 and fun34 genes for | 4.00E−06 |
| | | | DNA dependant RNA polymerase c (III) | |
| 1336 | 3762.E10.gz43_534137 | Z74847 | S. cerevisiae chromosome XV reading | 1.00E−05 |
| | | | frame ORF YOL105c | |
| 1337 | 3762.E15.gz43_534217 | AF207841 | Pyricularia grisea AVR-Pita (AVR-Pita) | 2.20E−09 |
| | | | gene, complete cds | |
| 1338 | 3762.E23.gz43_534345 | M58600 | Human heparin cofactor II (HCF2) gene, | 3.60E−37 |
| | | | exons 1 through 5 | |
| 1339 | 3762.F08.gz43_534106 | Z47066 | Human cosmid Qc14G3 from Xq28 | 3.10E−09 |
| | | | contains STSs | |
| 1340 | 3762.F22.gz43_534330 | AY034974 | Arabidopsis thaliana unknown protein | 4.20E−07 |
| | | | (F24J8.3) mRNA, complete cds | |
| 1341 | 3762.G18.gz43_534267 | Z28150 | S. cerevisiae chromosome XI reading | 2.00E−06 |
| | | | frame ORF YKL150w | |
| 1342 | 3762.H12.gz43_534172 | AF370230 | Arabidopsis thaliana unknown protein | 6.60E−08 |
| | | | (T21P5_16/AT3g03420) mRNA, | |
| | | | complete cds | |
| 1343 | 3762.I07.gz43_534093 | U19569 | Human squamous cell carcinoma antigen | 4.60E−07 |
| | | | (SCCA2) gene, exon 1 | |
| 1344 | 3762.J03.gz43_534030 | U22421 | Mus musculus obesity protein (ob) gene, | 5.30E−07 |
| | | | complete cds | |
| 1345 | 3762.J18.gz43_534270 | AB027966 | Schizosaccharomyces pombe gene for | 2.30E−08 |
| | | | Hypothetical protein, partial cds, | |
| | | | clone: TB89 | |
| 1346 | 3762.K02.gz43_534015 | AF273762 | Homo sapiens 3-hydroxy-3- | 4.40E−14 |
| | | | methylglutaryl-coenzyme reductase | |
| | | | gene, exon 15 | |
| 1347 | 3762.K20.gz43_534303 | K01464 | Rat cardiac alpha-myosin heavy chain | 3.00E−06 |
| | | | gene, 5′ flank, 1st 3 exons | |
| 1348 | 3762.L18.gz43_534272 | Z49438 | S. cerevisiae chromosome X reading | 4.00E−06 |
| | | | frame ORF YJL163c | |
| 1349 | 3762.L20.gz43_534304 | XM_030040 | Homo sapiens similar to KIAA0877 | 3.00E−06 |
| | | | protein (H. sapiens) (LOC90219), | |
| | | | mRNA | |
| 1350 | 3762.M04.gz43_534049 | AF002237 | Anopheles gambiae clone 227 mRNA | 4.00E−06 |
| | | | sequence | |
| 1351 | 3762.M17.gz43_534257 | M29688 | S. cerevisiae PMS1 gene encoding DNA | 1.40E−08 |
| | | | mismatch repair protein, complete cds | |
| 1352 | 3762.M23.gz43_534353 | M20006 | Chicken tumor 10 c-myc DNA, exons 2 | 2.90E−09 |
| | | | and 3 | |
| 1353 | Clu1014734.con_1 | AB027966 | Schizosaccharomyces pombe gene for | 3.00E−08 |
| | | | Hypothetical protein, partial cds, | |
| | | | clone: TB89 | |
| 1354 | Clu1036845.con_1 | M34429 | Human PVT-IGLC fusion protein | 1.37E−03 |
| | | | mRNA, 5′ end | |

### Example 21

### Members of Protein Families

SEQ ID NOS:134-1352 were used to conduct a profile search as described in the specification above. Several of the polynucleotides of the invention were found to encode polypeptides having characteristics of a polypeptide belonging to a known protein family (and thus represent members of these protein families) and/or comprising a known functional domain. Table 18 (inserted prior to claims) provides: 1) the SEQ ID NO (“SEQ ID”) of the query polynucleotide sequence; 2) the sequence name (“SEQ NAME”) used as an internal identifier of the query-sequence; 3) the name (“PFAM NAME”) of the profile hit; 4) a brief description of the profile hit (“PFAM DESCRIPTION”); 5) the score (“SCORE”) of the profile hit; 6) the starting nucleotide of the profile hit (“START”); and 7) the ending nucleotide of the profile hit (“END”).

TABLE 18

| SEQ | | | | | | |
|---|---|---|---|---|---|---|
| ID | SEQ NAME | PFAM NAME | PFAM DESCRIPTION | SCORE | START | END |
| 222 | 3547.D19.GZ43_505986 | DC1 | DC1 domain | 30.64 | 411 | 493 |
| 270 | 3550.G02.GZ43_506101 | rvt | Reverse transcriptase (RNA- | 47.32 | 321 | 611 |
| | | | dependent DNA polymerase) | | | |
| 454 | 3562.B22.GZ43_507952 | 7tm_1 | 7 transmembrane receptor | 37.16 | 154 | 479 |
| | | | (rhodopsin family) | | | |
| 454 | 3562.B22.GZ43_507952 | Bowman- | Bowman-Birk serine | 45.92 | 292 | 450 |
| | | Birk_leg | protease inhibitor family | | | |
| 454 | 3562.B22.GZ43_507952 | Cation_efflux | Cation efflux family | 33.32 | 225 | 380 |
| 491 | 3565.E16.GZ43_508243 | AP_endonucleas1 | AP endonuclease family 1 | 38.16 | 406 | 577 |
| 546 | 3571.A08.GZ43_508897 | oxidored_q1 | NADH- | 30.04 | 297 | 393 |
| | | | Ubiquinone/plastoquinone | | | |
| | | | (complex I), various chains | | | |
| 550 | 3571.B13.GZ43_508978 | EGF | EGF-like domain | 38.88 | 243 | 355 |
| 551 | 3571.B22.GZ43_509122 | EGF | EGF-like domain | 38.88 | 243 | 355 |
| 564 | 3571.H10.GZ43_508936 | WW | WW domain | 54.92 | 487 | 576 |
| 724 | 3583.H13.GZ43_510520 | Sre | C. elegans Sre G protein- | 30.36 | 282 | 485 |
| | | | coupled chemoreceptor | | | |
| 771 | 3590.J21.GZ43_512427 | bZIP | bZIP transcription factor | 33.68 | 166 | 308 |
| 778 | 3590.M03.GZ43_512142 | protamine_P1 | Protamine P1 | 35.88 | 268 | 437 |
| 907 | 3608.L14.gz43_514237 | Transposase_22 | L1 transposable element | 62.12 | 491 | 616 |
| 969 | 3617.P12.gz43_515361 | AP_endonucleas1 | AP endonuclease family 1 | 39.84 | 63 | 254 |
| 1038 | 3632.O06.gz43_517407 | 60s_ribosomal | 60s Acidic ribosomal protein | 38.04 | 276 | 444 |
| 1038 | 3632.O06.gz43_517407 | 60s_ribosomal | 60s Acidic ribosomal protein | 36.44 | 13 | 98 |
| 1128 | 3662.H23.gz43_520114 | Glycoprotein_G | Pneumovirus attachment | 43.04 | 21 | 297 |
| | | | glycoprotein G | | | |
| 1128 | 3662.H23.gz43_520114 | Metallothio_5 | Metallothionein family 5 | 47.88 | 231 | 345 |
| 1128 | 3662.H23.gz43_520114 | squash | Squash family serine | 34.6 | 222 | 301 |
| | | | protease inhibitor | | | |
| 1128 | 3662.H23.gz43_520114 | Syndecan | Syndecan domain | 35.36 | 1 | 308 |
| 1145 | 3663.G01.gz43_520145 | KRAB | KRAB box | 95.08 | 424 | 484 |
| 1350 | 3762.M04.gz43_534049 | protamine_P1 | Protamine P1 | 33.16 | 293 | 468 |

In addition, SEQ ID NOS:1619-1675 were also used to conduct a profile search as described above. Several of the polypeptides of the invention were found to have characteristics of a polypeptide belonging to a known protein family (and thus represent members of these protein families) and/or comprising a known functional domain. Table 19 (inserted prior to claims) provides: 1) the SEQ ID NO (“SEQ ID”) of the query protein sequence; 2) the sequence name (“PROTEIN SEQ NAME”) used as an internal identifier of the query sequence; 3) the name (“PFAM NAME”) of the profile hit; 4) a brief description of the profile hit (“PFAM DESCRIPTION”); 5) the score (“SCORE”) of the profile hit; 6) the starting residue of the profile hit (“START”); and 7) the ending residue of the profile hit (“END”).

TABLE 19

| SEQ | | PFAM | PFAM | | | |
|---|---|---|---|---|---|---|
| ID | SEQ NAME | NAME | DESCRIPTION | SCORE | START | END |
| 1619 | NTP_004511S11.3_4 | Armadillo_seg | Armadillo/beta- | 1.8E−95 | 142 | 184 |
| | | | catenin-like repeat | | | |
| 1619 | NTP_004511S11.3_4 | Armadillo_seg | Armadillo/beta- | 1.8E−95 | 186 | 226 |
| | | | catenin-like repeat | | | |
| 1619 | NTP_004511S11.3_4 | Armadillo_seg | Armadillo/beta- | 1.8E−95 | 228 | 269 |
| | | | catenin-like repeat | | | |
| 1619 | NTP_004511S11.3_4 | Armadillo_seg | Armadillo/beta- | 1.8E−95 | 271 | 311 |
| | | | catenin-like repeat | | | |
| 1619 | NTP_004511S11.3_4 | Armadillo_seg | Armadillo/beta- | 1.8E−95 | 313 | 353 |
| | | | catenin-like repeat | | | |
| 1619 | NTP_004511S11.3_4 | Armadillo_seg | Armadillo/beta- | 1.8E−95 | 355 | 395 |
| | | | catenin-like repeat | | | |
| 1619 | NTP_004511S11.3_4 | Armadillo_seg | Armadillo/beta- | 1.8E−95 | 397 | 437 |
| | | | catenin-like repeat | | | |
| 1619 | NTP_004511S11.3_4 | Armadillo_seg | Armadillo/beta- | 1.8E−95 | 440 | 480 |
| | | | catenin-like repeat | | | |
| 1619 | NTP_004511S11.3_4 | Armadillo_seg | Armadillo/beta- | 1.8E−95 | 142 | 184 |
| | | | catenin-like repeat | | | |
| 1619 | NTP_004511S11.3_4 | Armadillo_seg | Armadillo/beta- | 1.8E−95 | 186 | 226 |
| | | | catenin-like repeat | | | |
| 1619 | NTP_004511S11.3_4 | Armadillo_seg | Armadillo/beta- | 1.8E−95 | 228 | 269 |
| | | | catenin-like repeat | | | |
| 1619 | NTP_004511S11.3_4 | Armadillo_seg | Armadillo/beta- | 1.8E−95 | 271 | 311 |
| | | | catenin-like repeat | | | |
| 1619 | NTP_004511S11.3_4 | Armadillo_seg | Armadillo/beta- | 1.8E−95 | 313 | 353 |
| | | | catenin-like repeat | | | |
| 1619 | NTP_004511S11.3_4 | Armadillo_seg | Armadillo/beta- | 1.8E−95 | 355 | 395 |
| | | | catenin-like repeat | | | |
| 1619 | NTP_004511S11.3_4 | Armadillo_seg | Armadillo/beta- | 1.8E−95 | 397 | 437 |
| | | | catenin-like repeat | | | |
| 1619 | NTP_004511S11.3_4 | Armadillo_seg | Armadillo/beta- | 1.8E−95 | 440 | 480 |
| | | | catenin-like repeat | | | |
| 1619 | NTP_004511S11.3_4 | IBB | Importin beta binding | 5.8E−37 | 35 | 124 |
| | | | domain | | | |
| 1619 | NTP_004511S11.3_4 | IBB | Importin beta binding | 5.8E−37 | 35 | 124 |
| | | | domain | | | |
| 1630 | NTP_007592S2.3_10 | histone | Core histone | 1.2E−10 | 2 | 97 |
| | | | H2A/H2B/H3/H4 | | | |
| 1630 | NTP_007592S2.3_10 | histone | Core histone | 1.2E−10 | 2 | 97 |
| | | | H2A/H2B/H3/H4 | | | |
| 1633 | NTP_007867S7.3_3 | GTF2I | GTF2I-like repeat | 7.2E−76 | 106 | 171 |
| 1633 | NTP_007867S7.3_3 | GTF2I | GTF2I-like repeat | 7.2E−76 | 295 | 370 |
| 1633 | NTP_007867S7.3_3 | GTF2I | GTF2I-like repeat | 7.2E−76 | 106 | 171 |
| 1633 | NTP_007867S7.3_3 | GTF2I | GTF2I-like repeat | 7.2E−76 | 295 | 370 |
| 1634 | NTP_007867S8.3_1 | GTF2I | GTF2I-like repeat | 7.2E−76 | 122 | 187 |
| 1634 | NTP_007867S8.3_1 | GTF2I | GTF2I-like repeat | 7.2E−76 | 311 | 386 |
| 1634 | NTP_007867S8.3_1 | GTF2I | GTF2I-like repeat | 7.2E−76 | 122 | 187 |
| 1634 | NTP_007867S8.3_1 | GTF2I | GTF2I-like repeat | 7.2E−76 | 311 | 386 |
| 1640 | NTP_008858S2.3_2 | GST_N | Glutathione S- | 4.6E−11 | 21 | 95 |
| | | | transferase, N-terminal | | | |
| | | | domain | | | |
| 1640 | NTP_008858S2.3_2 | GST_N | Glutathione S- | 4.6E−11 | 21 | 95 |
| | | | transferase, N-terminal | | | |
| | | | domain | | | |
| 1643 | NTP_009526S2.3_3 | CBS | CBS domain | 4.8E−43 | 30 | 84 |
| 1643 | NTP_009526S2.3_3 | CBS | CBS domain | 4.8E−43 | 111 | 165 |
| 1643 | NTP_009526S2.3_3 | CBS | CBS domain | 4.8E−43 | 186 | 239 |
| 1643 | NTP_009526S2.3_3 | CBS | CBS domain | 4.8E−43 | 258 | 311 |
| 1643 | NTP_009526S2.3_3 | CBS | CBS domain | 4.8E−43 | 30 | 84 |
| 1643 | NTP_009526S2.3_3 | CBS | CBS domain | 4.8E−43 | 111 | 165 |
| 1643 | NTP_009526S2.3_3 | CBS | CBS domain | 4.8E−43 | 186 | 239 |
| 1643 | NTP_009526S2.3_3 | CBS | CBS domain | 4.8E−43 | 258 | 311 |
| 1644 | NTP_009526S2.3_5 | CBS | CBS domain | 4.8E−43 | 30 | 84 |
| 1644 | NTP_009526S2.3_5 | CBS | CBS domain | 4.8E−43 | 111 | 165 |
| 1644 | NTP_009526S2.3_5 | CBS | CBS domain | 4.8E−43 | 186 | 239 |
| 1644 | NTP_009526S2.3_5 | CBS | CBS domain | 4.8E−43 | 258 | 311 |
| 1644 | NTP_009526S2.3_5 | CBS | CBS domain | 4.8E−43 | 30 | 84 |
| 1644 | NTP_009526S2.3_5 | CBS | CBS domain | 4.8E−43 | 111 | 165 |
| 1644 | NTP_009526S2.3_5 | CBS | CBS domain | 4.8E−43 | 186 | 239 |
| 1644 | NTP_009526S2.3_5 | CBS | CBS domain | 4.8E−43 | 258 | 311 |
| 1647 | NTP_010018S2.3_5 | DAG_PE-bind | Phorbol | 7.7E−23 | 154 | 203 |
| | | | esters/diacylglycerol | | | |
| | | | binding domain (C1 | | | |
| | | | domain) | | | |
| 1647 | NTP_010018S2.3_5 | DAG_PE-bind | Phorbol | 7.7E−23 | 387 | 426 |
| | | | esters/diacylglycerol | | | |
| | | | binding domain (C1 | | | |
| | | | domain) | | | |
| 1647 | NTP_010018S2.3_5 | DAG_PE-bind | Phorbol | 7.7E−23 | 154 | 203 |
| | | | esters/diacylglycerol | | | |
| | | | binding domain (C1 | | | |
| | | | domain) | | | |
| 1647 | NTP_010018S2.3_5 | DAG_PE-bind | Phorbol | 7.7E−23 | 387 | 426 |
| | | | esters/diacylglycerol | | | |
| | | | binding domain (C1 | | | |
| | | | domain) | | | |
| 1651 | NTP_010757S4.3_2 | T-box | T-box | 6E−114 | 935 | 1099 |
| 1651 | NTP_010757S4.3_2 | T-box | T-box | 6E−114 | 1142 | 1160 |
| 1651 | NTP_010757S4.3_2 | T-box | T-box | 6E−114 | 935 | 1099 |
| 1651 | NTP_010757S4.3_2 | T-box | T-box | 6E−114 | 1142 | 1160 |
| 1653 | NTP_011130S2.3_3 | GATA | GATA zinc finger | 1.5E−11 | 159 | 198 |
| 1653 | NTP_011130S2.3_3 | GATA | GATA zinc finger | 1.5E−11 | 159 | 198 |
| 1656 | NTP_011430S6.3_6 | cadherin | Cadherin domain | 7.4E−61 | 174 | 270 |
| 1656 | NTP_011430S6.3_6 | cadherin | Cadherin domain | 7.4E−61 | 284 | 390 |
| 1656 | NTP_011430S6.3_6 | cadherin | Cadherin domain | 7.4E−61 | 405 | 495 |
| 1656 | NTP_011430S6.3_6 | cadherin | Cadherin domain | 7.4E−61 | 174 | 270 |
| 1656 | NTP_011430S6.3_6 | cadherin | Cadherin domain | 7.4E−61 | 284 | 390 |
| 1656 | NTP_011430S6.3_6 | cadherin | Cadherin domain | 7.4E−61 | 405 | 495 |
| 1658 | NTP_017582S2.3_6 | HMG_box | HMG (high mobility | 6.8E−09 | 34 | 92 |
| | | | group) box | | | |
| 1658 | NTP_017582S2.3_6 | HMG_box | HMG (high mobility | 6.8E−09 | 34 | 92 |
| | | | group) box | | | |
| 1675 | NTP_026331S1.1_1 | GTF2I | GTF2I-like repeat | 7.2E−76 | 106 | 171 |
| 1675 | NTP_026331S1.1_1 | GTF2I | GTF2I-like repeat | 7.2E−76 | 295 | 370 |
| 1675 | NTP_026331S1.1_1 | GTF2I | GTF2I-like repeat | 7.2E−76 | 106 | 171 |
| 1675 | NTP_026331S1.1_1 | GTF2I | GTF2I-like repeat | 7.2E−76 | 295 | 370 |

Some SEQ ID NOS exhibited multiple profile hits where the query sequence contains overlapping profile regions, and/or where the sequence contains two different functional domains. Each of the profile hits of Tables 18 and 19 is described in more detail below. The acronyms for the profiles (provided in parentheses) are those used to identify the profile in the Pfam, Prosite, and InterPro databases. The Pfam database can be accessed through web sites supported by Genome Sequencing Center at the Washington University School of Medicine or by the European Molecular Biology Laboratories in Heidelberg, Germany. The Prosite database can be accessed at the ExPASy Molecular Biology Server on the internet. The InterPro database can be accessed at a web site supported by the EMBL European Bioinformatics Institute. The public information available on the Pfam, Prosite, and InterPro databases regarding the various profiles, including but not limited to the activities, function, and consensus sequences of various proteins families and protein domains, is incorporated herein by reference.

Epidermal Growth Factor (EGF; Pfam Accession No. PF00008). SEQ ID NOS:550 and 551 represent polynucleotides encoding a member of the EGF family of proteins. The distinguishing characteristic of this family is the presence of a sequence of about thirty to forty amino acid residues found in epidermal growth factor (EGF) which has been shown to be present, in a more or less conserved form, in a large number of other proteins (Davis, New Biol. (1990) 2:410-419; Blomquist et al., Proc. Natl. Acad. Sci. U.S.A. (1984) 81:7363-7367; Barkert et al., Protein Nucl. Acid Enz. (1986) 29:54-86; Doolittle et al., Nature. (1984) 307:558-560; Appella et al., FEBS Lett. (1988) 231:1-4; Campbell and Bork, Curr. Opin. Struct. Biol. (1993) 3:385-392). A common feature of the domain is that the conserved pattern is generally found in the extracellular domain of membrane-bound proteins or in proteins known to be secreted. The EGF domain includes six cysteine residues which have been shown to be involved in disulfide bonds. The main structure is a two-stranded beta-sheet followed by a loop to a C-terminal short two-stranded sheet. Subdomains between the conserved cysteines strongly vary in length. These consensus patterns are used to identify members of this family: C-x-C-x(5)-G-x(2)-C and C-x-C-x(s)-[GP]-[FYW]-x(4,8)-C.

Seven Transmembrane Integral Membrane Proteins—Rhodopsin Family (7tm_{—}1; Pfam Accession No. PF00001). SEQ ID NO:454 corresponds to a sequence encoding a polypeptide that is a member of the seven transmembrane (7tm) receptor rhodopsin family. G-protein coupled receptors of the (7tm) rhodopsin family (also called R7G) are an extensive group of hormones, neurotransmitters, and light receptors which transduce extracellular signals by interaction with guanine nucleotide-binding (G) proteins (Strosberg, Eur. J. Biochem. (1991) 196:1; Kerlavage, Curr. Opin. Struct. Biol. (1991) 1:394; Probst et al., DNA Cell Biol. (1992) 11:1; Savarese et al., Biochem. J. (1992) 283:1. The consensus pattern that contains the conserved triplet and that also spans the major part of the third transmembrane helix is used to detect this widespread family of proteins: [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM].

Basic Region Plus Leucine Zipper Transcription Factors (bZIP; Pfam Accession No. PF00170). SEQ ID NO:771 represents a polynucleotide encoding a novel member of the family of basic region plus leucine zipper transcription factors. The bZIP superfamily (Hurst, Protein Prof. (1995) 2:105; and Ellenberger, Curr. Opin. Struct. Biol. (1994) 4:12) of eukaryotic DNA-binding transcription factors encompasses proteins that contain a basic region mediating sequence-specific DNA-binding followed by a leucine zipper required for dimerization. The consensus pattern for this protein family is: [KR]-x(1,3)-[RKSAQ]-N-x(2)-[SAQ](2)-x-[RKTAENQ]-x-R-x-[RK].

Reverse Transcriptase (rvt; Pfam Accession No. PF00078). SEQ ID NO:270 represents a polynucleotide encoding a reverse transcriptase, which occurs in a variety of mobile elements, including retrotransposons, retroviruses, group II introns, bacterial msDNAs, hepadnaviruses, and caulimoviruses (Xiong and Eickbush, EMBO J. (1990) 9:3353-3362). Reverse transcriptases catalyze RNA-template-directed extension of the 3′-end of a DNA strand by one deoxynucleotide at a time and require an RNA or DNA primer.

KRAB box (KRAB; Pfam Accession No. PF01352). SEQ ID NO:1145 represents a polypeptide having a Krueppel-associated box (KRAB). A KRAB box is a domain of around 75 amino acids that is found in the N-terminal part of about one third of eukaryotic Krueppel-type C₂H₂ zinc finger proteins (ZFPs). It is enriched in charged amino acids and can be divided into subregions A and B, which are predicted to fold into two amphipathic alpha-helices. The KRAB A and B boxes can be separated by variable spacer segments and many KRAB proteins contain only the A box.

The KRAB domain functions as a transcriptional repressor when tethered to the template DNA by a DNA-binding domain. A sequence of 45 amino acids in the KRAB A subdomain has been shown to be necessary and sufficient for transcriptional repression. The B box does not repress by itself but does potentiate the repression exerted by the KRAB A subdomain. Gene silencing requires the binding of the KRAB domain to the RING-B box-coiled coil (RBCC) domain of the KAP-1/TIF1-beta corepressor. As KAP-1 binds to the heterochromatin proteins HP1, it has been proposed that the KRAB-ZFP-bound target gene could be silenced following recruitment to heterochromatin.

KRAB-ZFPs constitute one of the single largest class of transcription factors within the human genome, and appear to play important roles during cell differentiation and development. The KRAB domain is generally encoded by two exons. The regions coded by the two exons are known as KRAB-A and KRAB-B.

Armadillo/beta-catenin-like repeat (Armadillo_seg: Pfam Accession No. PF00514). SEQ ID NO: 1619 represents a polypeptide having sequence similarity with the armadillo/beta-catenin-like repeat (armadillo). The armadillo repeat is an approximately 40 amino acid long tandemly repeated sequence motif first identified in the Drosophila segment polarity gene armadillo. Similar repeats were later found in the mammalian armadillo homolog beta-catenin, the junctional plaque protein plakoglobin, the adenomatous polyposis coli (APC) tumor suppressor protein, and a number of other proteins (Peifer et al., Cell 76(2):786-791 (1994)).

The 3 dimensional fold of an armadillo repeat is known from the crystal structure of beta-catenin (Rojas et al., Cell 95:105-130 (1998)). There, the 12 repeats form a superhelix of alpha-helices, with three helices per unit. The cylindrical structure features a positively charged grove which presumably interacts with the acidic surfaces of the known interaction partners of beta-catenin.

Cadherin domain (cadherin; Pfam Accession No. PF00028). SEQ ID NO: 1656 represents a polypeptide having sequence similarity to a cadherin domain. Cadherins are a family of animal glycoproteins responsible for calcium-dependent cell-cell adhesion (Takeichi, Annu. Rev. Biochem. 59:237-252(1990); Takeichi, Trends Genet. 3:213-217(1987)). Cadherins preferentially interact with themselves in a homophilic manner in connecting cells; thus acting as both receptor and ligand. A wide number of tissue-specific forms of cadherins are known, for example: Epithelial (E-cadherin) (CDH1); Neural (N-cadherin) (CDH2); Placental (P-cadherin) (CDH3); Retinal (R-cadherin) (CDH4); Vascular endothelial (VE-cadherin) (CDH5); Kidney (K-cadherin) (CDH6); Cadherin-8 (CDH8); Cadherin-9 (CDH9); Osteoblast (OB-cadherin) (CDH11); Brain (BR-cadherin) (CDH12); T-cadherin (truncated cadherin) (CDH13); Muscle (M-cadherin) (CDH15); Kidney (Ksp-cadherin) (CDH16); and Liver-intestine (LI-cadherin) (CDH17).

Structurally, cadherins are built of the following domains: a signal sequence, followed by a propeptide of about 130 residues, then an extracellular domain of around 600 residues, then a transmembrane region, and finally a C-terminal cytoplasmic domain of about 150 residues. The extracellular domain can be sub-divided into five parts: there are four repeats of about 110 residues followed by a region that contains four conserved cysteines. The calcium-binding region of cadherins may be located in the extracellular repeats. The signature pattern for the repeated domain is located in the C-terminal extremity, which is its best conserved region. The pattern includes two conserved aspartic acid residues and two asparagines; these residues could be implicated in the binding of calcium. The consensus pattern is: [LIV]-x-[LIV]-x-D-x-N-D-[NH]-x-P.

CBS domain (CBS; Pfam Accession No. PF00571). SEQ ID NOS:1643 and 1644 represent polypeptides having sequence similarity to CBS domains, which are present in all 3 forms of cellular life, including two copies in inosine monophosphate dehydrogenase, of which one is disordered in the crystal structure. A number of disease states are associated with CBS-containing proteins including homocystinuria, Becker's and Thomsen disease.

CBS domains are small intracellular modules of unknown function. They are mostly found in 2 or four copies within a protein. Pairs of CBS domains dimerise to form a stable globular domain (Zhang et al., Biochemistry 38:4691-4700 (1999)). Two CBS domains are found in inosine-monophosphate dehydrogenase from all species, however the CBS domains are not needed for activity. CBS domains are found attached to a wide range of other protein domains suggesting that CBS domains may play a regulatory role. The region containing the CBS domains in Cystathionine-beta synthase is involved in regulation by S-AdoMet (Zhang et al., Biochemistry 38:4691-4700 (1999)). The 3D Structure is found as a sub-domain in TIM barrel of inosine-monophosphate dehydrogenase.

Phorbol esters/diacylglycerol binding domain (C1 domain) (DAG_PE-bind: Pfam Accessin No. PF00130). SEQ ID NO: 1647 represents a polypeptide having sequence similarity to the Phorbol esters/diacylglycerol binding domain (C1 domain). Diacylglycerol (DAG) is an important second messenger. Phorbol esters (PE) are analogues of DAG and potent tumor promoters that cause a variety of physiological changes when administered to both cells and tissues. DAG activates a family of serine/threonine protein kinases, collectively known as protein kinase C (PKC) (Azzi et al., Eur. J. Biochem. 208:547-557 (1992)). Phorbol esters can also directly stimulate PKC.

The N-terminal region of PKC, known as C1, has been shown to bind PE and DAG in a phospholipid and zinc-dependent fashion (Ono et al., Proc. Natl. Acad. Sci. U.S.A. 86:4868-4871 (1989)). The C1 region contains one or two copies (depending on the isozyme of PKC) of a cysteine-rich domain about 50 amino-acid residues long and essential for DAG/PE-binding. The DAG/PE-binding domain binds two zinc ions; the ligands of these metal ions are probably the six cysteines and two histidines that are conserved in the C1 domain. The consensus sequence for the C1 domain is: H-x-[LIVMFYW]-x(8,11)-C-x(2)-C-x(3)-[LIVMFC]-x(5,10)-C-x(2)-C-x(4)-[HD]-x(2)-C-x(5,9)-C [All the C and H are involved in binding Zinc].

GATA zinc finger (GATA; Pfam Accession No. PF00320). SEQ ID NO:1653 represents a polypeptide having sequence similarity to GATA zinc finger. A number of transcription factors, including erythroid-specific transcription factor and nitrogen regulatory proteins, specifically bind the DNA sequence (A/T)GATA(A/G) in the regulatory regions of genes (Yamamoto et al., Genes Dev. 4:1650-1662 (1990)) and are consequently termed GATA-binding transcription factors. The interactions occur via highly-conserved zinc finger domains in which the zinc ion is coordinated by 4 cysteine residues (Evans and Felsenfeld, Cell 58:877-885 (1989); Omichinski et al., Science 261:438-446 (1993)).

NMR studies have shown the core of the zinc finger to comprise 2 irregular anti-parallel beta-sheets and an alpha-helix, followed by a long loop to the C-terminal end of the finger. The N-terminal part, which includes the helix, is similar in structure, but not sequence, to the N-terminal zinc module of the glucocorticoid receptor DNA-binding domain. The helix and the loop connecting the 2 beta-sheets interact with the major groove of the DNA, while the C-terminal tail wraps around into the minor groove. It is this tail that is the essential determinant of specific binding. Interactions between the zinc finger and DNA are mainly hydrophobic, explaining the preponderance of thymines in the binding site; a large number of interactions with the phosphate backbone have also been observed (Omichinski et al., Science 261:438-446 (1993)). Two GATA zinc fingers are found in the GATA transcription factors; however, there are several proteins which only contains a single copy of the domain. The consensus sequence of the domain is: C-x-[DN]-C-x(4,5)-[ST]-x(2)-W-[HR]-[RK]-x(3)-[GN]-x(3,4)-C-N-[AS]-C [The four C's are zinc ligands].

Glutathione S-transferase, N-terminal domain (GST_N: Pfam Accession No. PF02798). SEQ ID NO: 1640 represents a polypeptide having sequence similarity to Glutathione S-transferase, N-terminal domain. In eukaryotes, glutathione S-transferases (GSTs) participate in the detoxification of reactive electrophilic compounds by catalysing their conjugation to glutathione. The GST domain is also found in S-crystallins from squid, and proteins with no known GST activity, such as eukaryotic elongation factors 1-gamma and the HSP26 family of stress-related proteins, which include auxin-regulated proteins in plants and stringent starvation proteins in E. coli. The major lens polypeptide of Cephalopoda is also a GST.

Bacterial GSTs of known function often have a specific, growth-supporting role in biodegradative metabolism: epoxide ring opening and tetrachlorohydroquinone reductive dehalogenation are two examples of the reactions catalysed by these bacterial GSTs. Some regulatory proteins, like the stringent starvation proteins, also belong to the GST family. GST seems to be absent from Archaea in which gamma-glutamylcysteine substitute to glutathione as major thiol.

Glutathione S-transferases form homodimers, but in eukaryotes can also form heterodimers of the A1 and A2 or YC1 and YC2 subunits. The homodimeric enzymes display a conserved structural fold. Each monomer is composed of a distinct N-terminal sub-domain, which adopts the thioredoxin fold, and a C-terminal all-helical sub-domain.

GTF2I-like repeat (GTF2I; Pfam Accession No. PF02946). SEQ ID NOS:1633, 1634, and 1675 represent polypeptides having sequence similarity to proteins having GTF2I-like repeat. This region of sequence similarity is found up to six times in a variety of proteins including GTF2I. It has been suggested that this may be a DNA binding domain (O'Mahoney et al., Mol. Cell. Biol. 18:6641-6652 (1998); Osborne et al., Genomics 57:279-284 (1999)).

Core histone H2A/H2B/H3/H4 (histone; Pfam Accession No. PF00125). SEQ ID NO:1630 represents a polypeptide having sequence similarity to core histone H2A/H2B/H3/H4 family polypeptides. Histone H2A is one of the four histones, along with H2B, H3 and H4, which forms the eukaryotic nucleosome core. Using alignments of histone H2A sequences (Wells and Brown, Nucleic Acids Res. 19:2173-2188(1991); Thatcher and Gorovsky, Nucleic Acids Res. 22:174-179(1994)) a conserved region in the N-terminal part of H2A was used to develop a signature pattern. This region is conserved both in classical S-phase regulated H2A's and in variant histone H2A's which are synthesized throughout the cell cycle. The consensus pattern is: [AC]-G-L-x-F-P-V.

Histone H4, along with H3, plays a central role in nucleosome formation. The sequence of histone H4 has remained almost invariant in more then 2 billion years of evolution (Thatcher and Gorovsky, Nucleic Acids Res. 22:174-179(1994)). The region used as a signature pattern is a pentapeptide found in positions 14 to 18 of all H4 sequences. It contains a lysine residue which is often acetylated (Doenecke and Gallwitz, Mol. Cell. Biochem. 44:113-128(1982)) and a histidine residue which is implicated in DNA-binding (Ebralidse et al., Nature 331:365-367(1988)). The consensus pattern is: G-A-K-R-H.

Histone H3 is a highly conserved protein of 135 amino acid residues (Wells and Brown, Nucleic Acids Res. 19:2173-2188(1991); Thatcher and Gorovsky, Nucleic Acids Res. 22:174-179(1994)). Two signature patterns have been developed, the first one corresponds to a perfectly conserved heptapeptide in the N-terminal part of H3, while the second one is derived from a conserved region in the central section of H3. The consensus patterns are: K-A-P-R-K-Q-L and P-F-x-[RA]-L-[VA]-[KRQ]-[DEG]-[IV].

The signature pattern of histone H2B corresponds to a conserved region in the C-terminal part of the protein. The consensus pattern is: [KR]-E-[LIVM]-[EQ]-T-x(2)-[KR]-x-[LIVM](2)-x-[PAG]-[DE]-L-x-[KR]-H-A-[LIVM]-[STA]-E-G

HMG (high mobility group) box (HMG_box: Pfam Accession No. PF00505). SEQ ID NO:1658 corresponds to a polypeptide having sequence similarity to high mobility group proteins, a family of relatively low molecular weight non-histone components in chromatin. HMG1 (also called HMG-T in fish) and HMG2 (Bustin et al., Biochim. Biophys. Acta 1049: 231-243(1990)) are two highly related proteins that bind single-stranded DNA preferentially and unwind double-stranded DNA. HMG1/2 have about 200 amino acid residues with a highly acidic C-terminal section which is composed of an uninterrupted stretch of from 20 to 30 aspartic and glutamic acid residues; the rest of the protein sequence is very basic. In addition to the HMG1 and HMG2 proteins, HMG-domains occur in single or multiple copies in the following protein classes; the SOX family of transcription factors; SRY sex determining region Y protein and related proteins; LEF1 lymphoid enhancer binding factor 1; SSRP recombination signal recognition protein; MTF1 mitochondrial transcription factor 1; UBF1/2 nucleolar transcription factors; Abf2 yeast ARS-binding factor; and yeast transcription factors Ixr1, Rox1, Nhp6a, Nhp6b and Spp41.

Importin beta binding domain (IBB: Pfam Accession No. PF01749). SEQ ID NO: 1619 represents a polypeptide having sequence similarity to importin beta binding domain family polypeptides. This family consists of the importin alpha (karyopherin alpha), importin beta (karyopherin beta) binding domain. The domain mediates formation of the importin alpha beta complex; required for classical NLS import of proteins into the nucleus, through the nuclear pore complex and across the nuclear envelope. Also in the alignment is the NLS of importin alpha which overlaps with the IBB domain (Moroianu et al., Proc. Natl. Acad. Sci. U.S.A. 93:6572-6576(1996)).

T-box domain (T-box: Pfam Accession No. PF00907). SEQ ID NOS:1651 represents a polypeptide having sequence similarity to proteins having a T-box domain. The T-box gene family is an ancient group of putative transcription factors that appear to play a critical role in the development of all animal species. These genes were uncovered on the basis of similarity to the DNA binding domain (Papaioannou and Silver, Bioessays 20:9-19 (1998)) of murine Brachyury (T) gene product, which similarity is the defining feature of the family. The Brachyury gene is named for its phenotype, which was identified 70 years ago as a mutant mouse strain with a short blunted tail. The gene, and its paralogues, have become a well-studied model for the family, and hence much of what is known about the T-box family is derived from the murine Brachyury gene.

Consistent with its nuclear location, Brachyury protein has a sequence-specific DNA-binding activity and can act as a transcriptional regulator (Wattler et al., Genomics 48:24-33(1998)). Homozygous mutants for the gene undergo extensive developmental anomalies, thus rendering the mutation lethal (Kavka and Green, Biochim. Biophys. Acta 1333(2) (1997)). The postulated role of Brachyury is as a transcription factor, regulating the specification and differentiation of posterior mesoderm during gastrulation in a dose-dependent manner (Papaioannou and Silver, Bioessays 20:9-19 (1998)).

Common features shared by T-box family members are, DNA-binding and transcriptional regulatory activity, a role in development and conserved expression patterns. Most of the known genes in all species are expressed in mesoderm or mesoderm precursors (Papaioannou, Trends Genet. 13:212-213(1997)). Members of the T-box family contain a domain of about 170 to 190 amino acids known as the T-box domain (Papaioannou, Trends Genet. 13: 212-213(1997); Bollag et al., Nat. Genet. 7: 383-389(1994); Agulnik et al., Genetics 144:249-254(1996)) and which probably binds DNA. As signature patterns for the T-domain, we selected two conserved regions. The first region corresponds to the N-terminal of the domain and the second one to the central part. The consensus sequences are: L-W-x(2)-[FC]-x(3,4)-[NT]-E-M-[LIV](2)-T-x(2)-G-[RG]-[KRQ] and [LIVMFYW]-H-[PADH]-[DENQ]-[GS]-x(3)-G-x(2)-W-M-x(3)-[IVA]-x-F.

60s Acidic ribosomal protein (60s_ribosomal; Pfam Accession No. PF00428). SEQ ID NO: 1038 represents a polynucleotide encoding a member of the 60s acidic ribosomal protein family. The 60S acidic ribosomal protein plays an important role in the elongation step of protein synthesis. This family includes archaebacterial L12, eukaryotic P0, P1 and P2 (Remacha et al., Biochem. Cell Biol. 73:959-968(1995)).

Some of the proteins in this family are allergens. A nomenclature system has been established for antigens (allergens) that cause IgE-mediated atopic allergies in humans (WHO/IUIS Allergen Nomenclature Subcommittee King T. P., Hoffmann D., Loewenstein H., Marsh D. G., Platts-Mills T. A. E., Thomas W. Bull. World Health Organ. 72:797-806(1994)). This nomenclature system is defined by a designation that is composed of the first three letters of the genus; a space; the first letter of the species name; a space and an arabic number. In the event that two species names have identical designations, they are discriminated from one another by adding one or more letters (as necessary) to each species designation. The allergens in this family include allergens with the following designations: Alt a 6, Alt a 12, Cla h 3, Cla h 4, and Cla h 12.

AP endonuclease family 1 (AP_endonucleas1; Pfam Accession No. PF01260). SEQ ID NOS:491 and 969 correspond to a polynucleotide encoding a member of the family of polypeptides designated AP endonuclease family 1. DNA damaging agents such as the antitumor drugs bleomycin and neocarzinostatin or those that generate oxygen radicals produce a variety of lesions in DNA. Amongst these is base-loss which forms apurinic/apyrimidinic (AP) sites or strand breaks with atypical 3′-termini. DNA repair at the AP sites is initiated by specific endonuclease cleavage of the phosphodiester backbone. Such endonucleases are also generally capable of removing blocking groups from the 3′-terminus of DNA strand breaks.

AP endonucleases can be classified into two families on the basis of sequence similarity. This family contains members of AP endonuclease family 1. Except for Rrp1 and arp, these enzymes are proteins of about 300 amino-acid residues. Rrp1 and arp both contain additional and unrelated sequences in their N-terminal section (about 400 residues for Rrp1 and 270 for arp). The proteins contain glutamate which has been shown (Mol et al., Nature 374: 381-386(1995)), in the Escherichia coli enzyme to bind a divalent metal ion such as magnesium or manganese. The consensus sequences for this family of polypeptides are: [APF]-D-[LIVMF](2)-x-[LIVM]-Q-E-x-K [E binds a divalent metal ion]; D-[ST]-[FY]-R-[KH]-x(7,8)-[FYW]-[ST]-[FYW](2); and N-x-G-x-R-[LIVM]-D-[LIVMFYH]-x-[LV]-x-S

Bowman-Birk serine protease inhibitor family (Bowman-Birk_leg; Pfam Accession No. 00228). SEQ ID NO: 454 represents a polynucleotide encoding a polypeptide having sequence similarity to a member of the Bowman-Birk serine protease inhibitor family. The Bowman-Birk inhibitor family (Laskowski and Kato, Annu. Rev. Biochem. 49:593-626(1980)) is one of the numerous families of serine proteinase inhibitors and has a duplicated structure and generally possesses two distinct inhibitory sites.

These inhibitors are found in the seeds of all leguminous plants as well as in cereal grains. In cereals they exist in two forms, one of which is a duplication of the basic structure (Tashiro et al., J. Biochem. 102:297-306(1987)). The signature pattern for sequences belonging to this family of inhibitors is in the central part of the domain and includes four cysteines. The consensus pattern is: C-x(5,6)-[DENQKRHSTA]-C-[PASTDH]-[PASTDK]-[ASTDV]-C-[NDEKS]-[DEKRHSTA]-C [The four C's are involved in disulfide bonds]. Note that this pattern can be found twice in some duplicated cereal inhibitors.

Cation efflux family (Cation_efflux: Pfam Accession No. PF01545). SEQ ID NO: 454 encodes a polypeptide having sequence similarity to members of the cation efflux family of proteins. Members of this family are integral membrane proteins, that are found to increase tolerance to divalent metal ions such as cadmium, zinc, and cobalt. These proteins are thought to be efflux pumps that remove these ions from cells (Xiong and Jayaswal, J. Bacteriol. 180: 4024-4029(1998); Kunito et al, Biosci. Biotechnol. Biochem. 60: 699-704(1996)).

DC1 domain (DC1; Pfam Accession No. PF03107). SEQ ID NO: 222 corresponds to a polypeptide having sequence similarity to a DC1 domain. This short domain is rich in cysteines and histidines. The pattern of conservation is similar to that found in DAG_PE-bind (Pfam Accession No. PF00130), therefore this domain has been termed DC1 for divergent C1 domain. Like the DAG_PE-bind domain, this domain probably also binds to two zinc ions. The function of proteins with this domain is uncertain, however this domain may bind to molecules such as diacylglycerol. This family are found in plant proteins.

Pneumovirus attachment glycoprotein G (Glycoprotein_G; Pfam Accession No. PF00802). SEQ ID NO:1128 represents a polypeptide having sequence similarity to members of the Pneumovirus attachment glycoprotein G protein family. This family includes attachment proteins from respiratory synctial virus. Glycoprotein G has not been shown to have any neuramimidase or hemagglutinin activity. The amino terminus is thought to be cytoplasmic, and the carboxyl terminus extracellular. The extracellular region contains four completely conserved cysteine residues.

NADH-Ubiquinone/plastoquinone (complex I), various chains (oxidored_q 1; Pfam Accession No. PF00361). SEQ ID NO:546 represents a polypeptide having sequence similarity to NADH-Ubiquinone/plastoquinone (complex I), various chains protein family. This family is part of the NADH:ubiquinone oxidoreductase (complex I) which catalyses the transfer of two electrons from NADH to ubiquinone in a reaction that is associated with proton translocation across the membrane (Walker, Q. Rev. Biophys. 25: 253-324(1992)). Sub-families within this protein family include NADH-ubiquinone oxidoreductase chain 5; NADH-ubiquinone oxidoreductase chain 2; NADH-ubiquinone oxidoreductase chain 4; and Multicomponent K+:H+antiporter.

Protamine P1 (protamine_P1; Pfam Accession No. PF00260). SEQ ID NOS:778 and 1450 represent polypeptides having sequence similarity to Protamine P1 protein family. Protamines are small, highly basic proteins, that substitute for histones in sperm chromatin during the haploid phase of spermatogenesis. They pack sperm DNA into a highly condensed, stable and inactive complex. There are two different types of mammalian protamine, called P1 and P2. P1 has been found in all species studied, while P2 is sometimes absent. There also seems to be a single type of avian protamine whose sequence is closely related to that of mammalian P1 (Oliva et al., J. Biol. Chem. 264:17627-17630(1989)). A conserved region at the N-terminal extremity of the sequence is used as a signature pattern for this family of proteins. The consensus pattern is: [AV]-R-[NFY]-R-x(2,3)-[ST]-x-S-x-S.

Squash family serine protease inhibitor (squash; Pfam Accession No. PF00299). SEQ ID NO:1128 represents a polypeptide having sequence similarity to Squash family serine protease inhibitor proteins. The squash inhibitors form one of a number of serine protease inhibitor families. The proteins, found in the seeds of cucurbitaceae plants (squash, cucumber, balsam pear, etc.), are approximately 30 residues in length, and contain 6 Cys residues, which form 3 disulfide bonds (Bode et al., FEBS Lett. 242: 285-292(1989)). The inhibitors function by being taken up by a serine protease (such as trypsin), which cleaves the peptide bond between Arg/Lys and Ile residues in the N-terminal portion of the protein (Bode et al., FEBS Lett. 242: 285-292(1989); Krishnamoorthi et al., Biochemistry 31: 898-904(1992)). Structural studies have shown that the inhibitor has an ellipsoidal shape, and is largely composed of beta-turns (Bode et al., FEBS Lett. 242: 285-292(1989)). The fold and Cys connectivity of the proteins resembles that of potato carboxypeptidase A inhibitor (Krishnamoorthi et al., Biochemistry 31: 898-904(1992)). The pattern used to detect this family of proteins spans the major part of the sequence and includes five of the six cysteines involved in disulfide bonds. The consensus pattern is: C-P-x(5)-C-x(2)-[DN]-x-D-C-x(3)-C-x-C [The five C's are involved in disulfide bonds]

Metallothionein family 5 (Metallothio_{—}5: Pfam Accession No. PF02067). SEQ ID NO:1128 represents a polypeptide having sequence similarity to metallothionein family 5 proteins. Metallothioneins (MT) are small proteins that bind heavy metals, such as zinc, copper, cadmium, and nickel. They have a high content of cysteine residues that bind the metal ions through clusters of thiolate bonds (Kagi, Meth. Enzymol. 205: 613-626(1991); Kagi and Kojima, Experientia Suppl. 52: 25-61(1987); Kagi and Schaffer, Biochemistry 27: 8509-8515(1988)).

Due to limitations in the original classification system of MTs, which did not allow clear differentiation of patterns of structural similarities, either between or within classes, all class I and class II MTs (the proteinaceous sequences) have now been grouped into families of phylogenetically-related and thus alignable sequences. Diptera (Drosophila, family 5) MTs are 40-43 residue proteins that contain 10 conserved cysteines arranged in five Cys-X-Cys groups. In particular, the consensus pattern C-G-x(2)-C-x-C-x(2)-Q-x(5)-C-x-C-x(2)-D-C-x-C has been found to be diagnostic of family 5 MTs. The protein is found primarily in the alimentary canal, and its induction is stimulated by ingestion of cadmium or copper (Lastowski et al., J. Biol. Chem. 260: 1527-1530(1985)). Mercury, silver and zinc induce the protein to a lesser extent.

Caenorhabditis. elegans Sre G protein-coupled chemoreceptor (Sre; Pfam Accession No. PF03125). SEQ ID NO:724 represents a polypeptide having sequence similarity to C. elegans Sre G protein-coupled chemoreceptor family proteins. C. elegans Sre proteins are candidate chemosensory receptors. There are four main recognized groups of such receptors: Odr-10, Sra, Sro, and Srg. Sre (this family), Sra Sra and Srb Srb comprise the Sra group. All of the above receptors are thought to be G protein-coupled seven transmembrane domain proteins (Troemel, Bioessays 21:1011-1020 (1999); Troemel et al., Cell 83:207-218 (1995)).

Syndecan domain (Syndecan; Pfam Accession No. PF01034). SEQ ID NO:1128 corresponds to a polypeptide having a syndecan domain. Syndecans (Bernfield et al., Annu. Rev. Cell Biol. 8:365-393(1992); David, FASEB J. 7:1023-1030(1993)) are a family of transmembrane heparan sulfate proteoglycans which are implicated in the binding of extracellular matrix components and growth factors. Syndecans bind a variety of molecules via their heparan sulfate chains and can act as receptors or as co-receptors. Structurally, these proteins consist of four separate domains: a) a signal sequence; b) an extracellular domain (ectodomain) of variable length containing the sites of attachment of the heparan sulfate glycosaminoglycan side chains and whose sequence is not evolutionarily conserved in the various forms of syndecans; c) a transmembrane region; and d) a highly conserved cytoplasmic domain of about 30 to 35 residues which could interact with cytoskeletal proteins.

The signature pattern for syndecans starts with the last residue of the transmembrane region and includes the first 10 residues of the cytoplasmic domain. This region, which contains four basic residues, may act as a stop transfer site. The consensus pattern is: [FY]-R-[IM]-[KR]-K(2)-D-E-G-S-Y.

L1 transposable element (Transposase_{—}22; Pfam Accession No. PF02994). SEQ ID NO:907 represents a polypeptide having an L1 transposable element. Many human L1 elements are capable of retrotransposition and some of these have been shown to exhibit reverse transcriptase (RT) activity (Sassaman et al., Nat Genet 16(1):37-43(1997)) although the function of many are, as yet, unknown. There are estimated to be 30-60 active L1 elements reside in the average diploid genome.

WW domain (WW; Pfam Accession No. PF00397). SEQ ID NO:564 represents a polypeptide having WW domain. The WW domain (also known as rsp5 or WWP) is a short conserved region in a number of unrelated proteins, among them dystrophin, responsible for Duchenne muscular dystrophy. This short domain may be repeated up to four times in some proteins (Bork and Sudol, Trends Biochem. Sci. 19: 531-533(1994); Andre and Springael, Biochem. Biophys. Res. Commun. 205: 1201-1205(1994); Hofmann and Bucher, FEBS Lett. 358: 153-157(1995); Sudol et al., FEBS Lett. 369: 67-71(1995)). The WW domain binds to proteins with particular proline-motifs, [AP]-P-P-[AP]-Y, and having four conserved aromatic positions that are generally Trp (Chen and Sudol, Proc. Natl. Acad. Sci. U.S.A. 92: 7819-7823(1995)). The name WW or WWP derives from the presence of these Trp as well as that of a conserved Pro. The WW domain is frequently associated with other domains typical for proteins in signal transduction processes.

A large variety of proteins containing the WW domain are known. These include; dystrophin, a multidomain cytoskeletal protein; utrophin, a dystrophin-like protein of unknown function; vertebrate YAP protein, substrate of an unknown serine kinase; mouse NEDD-4, involved in the embryonic development and differentiation of the central nervous system; yeast RSP5, similar to NEDD-4 in its molecular organization; rat FE65, a transcription-factor activator expressed preferentially in liver; tobacco DB10 protein and others. The consensus pattern is: W-x(9,11)-[VFY]-[FYW]-x(6,7)-[GSTNE]-[GSTQCR]-[FYW]-x(2)-P.

### Example 22

### Detection of Differential Expression Using Arrays and Source of Patient Tissue Samples

mRNA isolated from samples of cancerous and normal breast and colon tissue obtained from patients were analyzed to identify genes differentially expressed in cancerous and normal cells. Normal and cancerous tissues were collected from patients using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001).

Table 20 (inserted prior to claims) provides information about each patient from which colon tissue samples were isolated, including: the Patient ID (“PT ID”) and Path ReportID (“Path ID”), which are numbers assigned to the patient and the pathology reports for identification purposes; the group (“Grp”) to which the patients have been assigned; the anatomical location of the tumor (“Anatom Loc”); the primary tumor size (“Size”); the primary tumor grade (“Grade”); the identification of the histopathological grade (“Histo Grade”); a description of local sites to which the tumor had invaded (“Local Invasion”); the presence of lymph node metastases (“Lymph Met”); the incidence of lymph node metastases (provided as a number of lymph nodes positive for metastasis over the number of lymph-nodes examined) (“Lymph Met Incid”); the regional lymphnode grade (“Reg Lymph Grade”); the identification or detection of metastases to sites distant to the tumor and their location (“Dist Met & Loc”); the grade of distant metastasis (“Dist Met Grade”); and general comments about the patient or the tumor (“Comments”). Histophatology of all primary tumors indicated the tumor was adenocarcinmoa except for Patient ID Nos. 130 (for which no information was provided), 392 (in which greater than 50% of the cells were mucinous carcinoma), and 784 (adenosquamous carcinoma). Extranodal extensions were described in three patients, Patient ID Nos. 784, 789, and 791. Lymphovascular invasion was described in Patient ID Nos. 128, 278, 517, 534, 784, 786, 789, 791, 890, and 892. Crohn's-like infiltrates were described in seven patients, Patient ID Nos. 52, 264, 268, 392, 393, 784, and 791. Table 21 (below) provides information about each patient from which the breast tissue samples were isolated, including: 1) the “Pat Num”, a number assigned to the patient for identification purposes; 2) the “Histology”, which indicates whether the tumor was characterized as an intraductal carcinoma (IDC) or ductal carcinoma in situ (DCIS); 3) the incidence of lymph node metastases (LMF), represented as the number of lymph nodes positive to metastases out of the total number examined in the patient; 4) the “Tumor Size”; 5) “TNM Stage”, which provides the tumor grade (T#), where the number indicates the grade and “p” indicates that the tumor grade is a pathological classification; regional lymph node metastasis (N#), where “0” indicates no lymph node metastases were found, “1” indicates lymph node metastases were found, and “X” means information not available and; the identification or detection of metastases to sites distant to the tumor and their location (M#), with “X” indicating that no distant mesatses were reported; and the stage of the tumor (“Stage Grouping”). “nr” indicates “no reported”.

TABLE 20

| | | | | | | | | | Lymph | Reg | Dist | Dist | |
|---|---|---|---|---|---|---|---|---|---|---|---|---|---|
| | Path | | Anatom | | | Histo | | Lymph | Met | Lymph | Met & | Met | |
| Pt ID | ID | Grp | Loc | Size | Grade | Grade | Local Invasion | Met | Incid | Grade | Loc | Grade | Comment |
| 15 | 21 | III | Ascending | 4.0 | T3 | G2 | Extending into | Pos | 3/8 | N1 | Neg | MX | invasive |
| | | | colon | | | | subserosal adipose | | | | | | adenocarcinoma, |
| | | | | | | | tissue | | | | | | moderately |
| | | | | | | | | | | | | | differentiated; |
| | | | | | | | | | | | | | focal perineural |
| | | | | | | | | | | | | | invasion is seen |
| 52 | 71 | II | Cecum | 9.0 | T3 | G3 | Invasion through | Neg | 0/12 | N0 | Neg | M0 | Hyperplastic |
| | | | | | | | muscularis | | | | | | polyp in |
| | | | | | | | propria, subserosal | | | | | | appendix. |
| | | | | | | | involvement; | | | | | | |
| | | | | | | | ileocec. valve | | | | | | |
| | | | | | | | involvement | | | | | | |
| 121 | 140 | II | Sigmoid | 6 | T4 | G2 | Invasion of | Neg | 0/34 | N0 | Neg | M0 | Perineural |
| | | | | | | | muscularis propria | | | | | | invasion; donut |
| | | | | | | | into serosa, | | | | | | anastomosis |
| | | | | | | | involving | | | | | | Neg. One |
| | | | | | | | submucosa of | | | | | | tubulovillous |
| | | | | | | | urinary bladder | | | | | | and one tubular |
| | | | | | | | | | | | | | adenoma with |
| | | | | | | | | | | | | | no high grade |
| | | | | | | | | | | | | | dysplasia. |
| 125 | 144 | II | Cecum | 6 | T3 | G2 | Invasion through | Neg | 0/19 | N0 | Neg | M0 | patient history |
| | | | | | | | the muscularis | | | | | | of metastatic |
| | | | | | | | propria into | | | | | | melanoma |
| | | | | | | | suserosal adipose | | | | | | |
| | | | | | | | tissue. Ileocecal | | | | | | |
| | | | | | | | junction. | | | | | | |
| 128 | 147 | III | Transverse | 5.0 | T3 | G2 | Invasion of | Pos | 1/5 | N1 | Neg | M0 | |
| | | | colon | | | | muscularis propria | | | | | | |
| | | | | | | | into percolonic fat | | | | | | |
| 130 | 149 | | Splenic | 5.5 | T3 | | through wall and | Pos | 10/24 | N2 | Neg | M1 | |
| | | | flexure | | | | into surrounding | | | | | | |
| | | | | | | | adipose tissue | | | | | | |
| 133 | 152 | II | Rectum | 5.0 | T3 | G2 | Invasion through | Neg | 0/9 | N0 | Neg | M0 | Small separate |
| | | | | | | | muscularis propria | | | | | | tubular |
| | | | | | | | into non- | | | | | | adenoma (0.4 cm) |
| | | | | | | | peritonealized | | | | | | |
| | | | | | | | pericolic tissue; | | | | | | |
| | | | | | | | gross | | | | | | |
| | | | | | | | configuration is | | | | | | |
| | | | | | | | annular. | | | | | | |
| 141 | 160 | IV | Cecum | 5.5 | T3 | G2 | Invasion of | Pos | 7/21 | N2 | Pos - | M1 | Perineural |
| | | | | | | | muscularis propria | | | | Liver | | invasion |
| | | | | | | | into pericolonic | | | | | | identified |
| | | | | | | | adipose tissue, but | | | | | | adjacent to |
| | | | | | | | not through serosa. | | | | | | metastatic |
| | | | | | | | Arising from | | | | | | adenocarcinoma. |
| | | | | | | | tubular adenoma. | | | | | | |
| 156 | 175 | III | Hepatic | 3.8 | T3 | G2 | Invasion through | Pos | 2/13 | N1 | Neg | M0 | Separate |
| | | | flexure | | | | muscularis propria | | | | | | tubolovillous |
| | | | | | | | into | | | | | | and tubular |
| | | | | | | | subserosa/pericolic | | | | | | adenomas |
| | | | | | | | adipose, no serosal | | | | | | |
| | | | | | | | involvement. | | | | | | |
| | | | | | | | Gross | | | | | | |
| | | | | | | | configuration | | | | | | |
| | | | | | | | annular. | | | | | | |
| 228 | 247 | III | Rectum | 5.8 | T3 | G2 to | Invasion through | Pos | 1/8 | N1 | Neg | MX | Hyperplastic |
| | | | | | | G3 | muscularis propria | | | | | | polyps |
| | | | | | | | to involve | | | | | | |
| | | | | | | | subserosal, | | | | | | |
| | | | | | | | perirectoal | | | | | | |
| | | | | | | | adipose, and | | | | | | |
| | | | | | | | serosa | | | | | | |
| 264 | 283 | II | Ascending | 5.5 | T3 | G2 | Invasion through | Neg | 0/10 | N0 | Neg | M0 | Tubulovillous |
| | | | colon | | | | muscularis propria | | | | | | adenoma with |
| | | | | | | | into subserosal | | | | | | high grade |
| | | | | | | | adipose tissue. | | | | | | dysplasia |
| 266 | 285 | III | Transverse | 9 | T3 | G2 | Invades through | Neg | 0/15 | N1 | Pos - | MX | |
| | | | colon | | | | muscularis propria | | | | Mesen- | | |
| | | | | | | | to involve | | | | teric | | |
| | | | | | | | pericolonic | | | | deposit | | |
| | | | | | | | adipose, extends to | | | | | | |
| | | | | | | | serosa. | | | | | | |
| 268 | 287 | I | Cecum | 6.5 | T2 | G2 | Invades full | Neg | 0/12 | N0 | Neg | M0 | |
| | | | | | | | thickness of | | | | | | |
| | | | | | | | muscularis | | | | | | |
| | | | | | | | propria, but | | | | | | |
| | | | | | | | mesenteric adipose | | | | | | |
| | | | | | | | free of malignancy | | | | | | |
| 278 | 297 | III | Rectum | 4 | T3 | G2 | Invasion into | Pos | 7/10 | N2 | Neg | M0 | Descending |
| | | | | | | | perirectal adipose | | | | | | colon polyps, |
| | | | | | | | tissue. | | | | | | no HGD or |
| | | | | | | | | | | | | | carcinoma |
| | | | | | | | | | | | | | identified. |
| 296 | 315 | III | Cecum | 5.5 | T3 | G2 | Invasion through | Pos | 2/12 | N1 | Neg | M0 | Tubulovillous |
| | | | | | | | muscularis propria | | | | | | adenoma (2.0 cm) |
| | | | | | | | and invades | | | | | | with no |
| | | | | | | | pericolic adipose | | | | | | high grade |
| | | | | | | | tissue. Ileocecal | | | | | | dysplasia. Neg. |
| | | | | | | | junction. | | | | | | liver biopsy. |
| 339 | 358 | II | Recto- | 6 | T3 | G2 | Extends into | Neg | 0/6 | N0 | Neg | M0 | 1 hyperplastic |
| | | | sigmoid | | | | perirectal fat but | | | | | | polyp identified |
| | | | | | | | does not reach | | | | | | |
| | | | | | | | serosa | | | | | | |
| 341 | 360 | II | Ascending | 2 cm | T3 | G2 | Invasion through | Neg | 0/4 | N0 | Neg | MX | |
| | | | colon | invasive | | | muscularis propria | | | | | | |
| | | | | | | | to involve | | | | | | |
| | | | | | | | pericolonic fat. | | | | | | |
| | | | | | | | Arising from | | | | | | |
| | | | | | | | villous adenoma. | | | | | | |
| 356 | 375 | II | Sigmoid | 6.5 | T3 | G2 | Through colon | Neg | 0/4 | N0 | Neg | M0 | |
| | | | | | | | wall into | | | | | | |
| | | | | | | | subserosal adipose | | | | | | |
| | | | | | | | tissue. No serosal | | | | | | |
| | | | | | | | spread seen. | | | | | | |
| 360 | 412 | III | Ascending | 4.3 | T3 | G2 | Invasion thru | Pos | 1/5 | N1 | Neg | M0 | Two mucosal |
| | | | colon | | | | muscularis propria | | | | | | polyps |
| | | | | | | | to pericolonic fat | | | | | | |
| 392 | 444 | IV | Ascending | 2 | T3 | G2 | Invasion through | Pos | 1/6 | N1 | Pos - | M1 | Tumor arising |
| | | | colon | | | | muscularis propria | | | | Liver | | at prior |
| | | | | | | | into subserosal | | | | | | ileocolic |
| | | | | | | | adipose tissue, not | | | | | | surgical |
| | | | | | | | serosa. | | | | | | anastomosis. |
| 393 | 445 | II | Cecum | 6.0 | T3 | G2 | Cecum, invades | Neg | 0/21 | N0 | Neg | M0 | |
| | | | | | | | through | | | | | | |
| | | | | | | | muscularis propria | | | | | | |
| | | | | | | | to involve | | | | | | |
| | | | | | | | subserosal adipose | | | | | | |
| | | | | | | | tissue but not | | | | | | |
| | | | | | | | serosa. | | | | | | |
| 413 | 465 | IV | Cecum | 4.8 | T3 | G2 | Invasive through | Neg | 0/7 | N0 | Pos - | M1 | rediagnosis of |
| | | | | | | | muscularis to | | | | Liver | | oophorectomy |
| | | | | | | | involve periserosal | | | | | | path to |
| | | | | | | | fat; abutting | | | | | | metastatic |
| | | | | | | | ileocecal junction. | | | | | | colon cancer. |
| 505 | 383 | IV | | 7.5 | T3 | G2 | Invasion through | Pos | 2/17 | N1 | Pos - | M1 | Anatomical |
| | | | | | | | muscularis propria | | | | Liver | | location of |
| | | | | | | | involving pericolic | | | | | | primary not |
| | | | | | | | adipose, serosal | | | | | | notated in |
| | | | | | | | surface uninvolved | | | | | | report. |
| | | | | | | | | | | | | | Evidence of |
| | | | | | | | | | | | | | chronic colitis. |
| 517 | 395 | IV | Sigmoid | 3 | T3 | G2 | penetrates | Pos | 6/6 | N2 | Neg | M0 | No mention of |
| | | | | | | | muscularis | | | | | | distant met in |
| | | | | | | | propria, involves | | | | | | report |
| | | | | | | | pericolonic fat. | | | | | | |

TABLE 21
Breast cancer patient data.

| Pat | | | Tumor | | |
|---|---|---|---|---|---|
| Num | Histology | LMF | Size | TNM Stage | Stage Grouping |
| 280 | IDC, DCIS + | nr | 2 cm | T2NXMX | probable Stage II |
| | D2 | | | | |
| 284 | IDC, DCIS | 0/16 | 2 cm | T2pN0MX | Stage II |
| 285 | IDC, DCIS | nr | 4.5 cm | T2NXMX | probable Stage II |
| 291 | IDC, DCIS | 0/24 | 4.5 cm | T2pN0MX | Stage II |
| 302 | IDC, DCIS | nr | 2.2 cm | T2NXMX | probable Stage II |
| 375 | IDC, DCIS | nr | 1.5 cm | T1NXMX | probable Stage I |
| 408 | IDC | 0/23 | 3.0 cm | T2pN0MX | Stage II |
| 416 | IDC | 0/6 | 3.3 cm | T2pN0MX | Stage II |
| 421 | IDC, DCIS | nr | 3.5 cm | T2NXMX | probable Stage II |
| 459 | IDC | 2/5 | 4.9 cm | T2pN1MX | Stage II |
| 465 | IDC | 0/10 | 6.5 cm | T3pN0MX | Stage II |
| 470 | IDC, DCIS | 0/6 | 2.5 cm | T2pN0MX | Stage II |
| 472 | IDC, DCIS | 6/45 | 5.0+ cm | T3pN1MX | Stage III |
| 474 | IDC | 0/18 | 6.0 cm | T3pN0MX | Stage II |
| 476 | IDC | 0/16 | 3.4 cm | T2pN0MX | Stage II |
| 605 | IDC, DCIS | 1/25 | 5.0 cm | T2pN1MX | Stage II |
| 649 | IDC, DCIS | 1/29 | 4.5 cm | T2pN1MX | Stage II |

Identification of Differentially Expressed Genes

cDNA probes were prepared from total RNA isolated from the patient cells described above. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was the transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red), and vice versa.

Each array used had an identical spatial layout and control spot set. Each microarray was divided into two areas, each area having an array with, on each half, twelve groupings of 32×12 spots, for a total of about 9,216 spots on each array. The two areas are spotted identically which provide for at least two duplicates of each clone per array.

Polynucleotides for use on the arrays were obtained from both publicly available sources and from cDNA libraries generated from selected cell lines and patient tissues. PCR products of from about 0.5 kb to 2.0 kb amplified from these sources were spotted onto the array using a Molecular Dynamics Gen III spotter according to the manufacturer's recommendations. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about four duplicate measurements for each clone, two of one color and two of the other, for each sample.

The differential expression assay was performed by mixing equal amounts of probes from tumor cells and normal cells of the same patient (“matched”) or from tumor cells and normal cells of different patients (“unmatched”) (i.e., the tumor cells are from one patient and the normal cells are from a different patient). The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1×SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient in matched samples or between tumor and normal samples of tissue from different patients in unmatched samples. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

Table 22 (inserted prior to claims) provides the results for gene products expressed by at least 2-fold or greater in cancerous prostate, colon, or breast tissue samples relative to normal tissue samples in at least 20% of the patients tested. Table 22 includes: 1) the SEQ ID NO (“SEQ ID”) assigned to each sequence for use in the present specification; 2) the sequence name (“SEQ NAME”) used as an internal identifier of the sequence; 3) the name assigned to the clone from which the sequence was isolated (“CLONE ID”); 4) the percentage of patients tested in which expression levels (e.g., as message level) of the gene was at least 2-fold greater in cancerous breast tissue than in matched normal tissue (“BREAST PATIENTS >=2×”); 5) the breast number ratios, indicating the number of patients upon which the provided ratio using matched breast tissue was based (“BREAST NUM RATIOS”); 6) the percentage of patients tested in which expression levels (e.g., as message level) of the gene was at least 2-fold greater in cancerous colon tissue than in matched normal tissue (“COLON PATIENTS >=2×”); 7) the colon number ratios, indicating the number of patients upon which the provided ratio using matched colon tissue was based (“COLON NUM RATIOS”); 8) the percentage of patients tested in which expression levels (e.g., as message level) of the gene was at least 2-fold greater in cancerous colon tissue than in unmatched normal tissue (“COLON UM>=2×”); 9) the unmatched colon number ratios, indicating the number of patients upon which the provided ratio using unmatched colon tissue was based (“COLON UM NUM RATIOS”).

TABLE 22

| | | | | | | | | COLON |
|---|---|---|---|---|---|---|---|---|
| | | | BREAST | BREAST | COLON | COLON | | UM |
| SEQ | | | PATIENTS | NUM | PATIENTS | NUM | COLON | NUM |
| ID | SEQ NAME | CLONE ID | >=2x | RATIOS | >=2x | RATIOS | UM >=2x | RATIOS |
| 189 | 3544.G06.GZ43_505397 | M00084443A:E10 | | | 50 | 8 | | |
| 420 | 3559.B18.GZ43_507504 | M00084700A:C10 | | | 41.025641 | 39 | 33.33333 | 27 |
| 754 | 3590.D19.GZ43_512389 | M00085031B:E03 | | | 37.5 | 8 | | |
| 816 | 3596.P03.GZ43_512529 | M00085171D:F05 | | | 70 | 40 | 60.71429 | 28 |
| 839 | 3599.K02.GZ43_512892 | M00085222D:D07 | | | 70 | 40 | 60.71429 | 28 |
| 1192 | 3665.O06.gz43_521001 | M00086277B:E06 | | | 57.5 | 40 | 50 | 12 |
| 1283 | 3756.K15.gz43_533455 | M00085835B:E11 | | | 35.2941176 | 34 | 35.71429 | 28 |
| 1289 | 3756.M06.gz43_533313 | M00085815C:E11 | 47.0588235 | 17 | | | | |
| 1320 | 3759.P15.gz43_533844 | M00085100B:C12 | | | 63.4146341 | 41 | | |
| 1549 | NT_007592S2.3_10 | | 50 | 10 | | | | |
| 1560 | NT_009296S1.3_1 | | | | | | 42.9 | 28 |
| 1560 | NT_009296S1.3_1 | | | | 46.2 | 39 | 33.3 | 27 |
| 1560 | NT_009296S1.3_1 | | | | 48.7 | 39 | 44.4 | 27 |
| 1577 | NT_017582S2.3_6 | | | | 61.5 | 39 | 55.6 | 27 |
| 1577 | NT_017582S2.3_6 | | | | 61.5 | 39 | 55.6 | 27 |

Table 25 (inserted prior to claims) provides the results for other gene products expressed by at least 2-fold or greater in cancerous prostate, colon, or breast tissue sample, which may be metastasized cancer samples, relative to normal tissue samples in at least 20% of the patients tested. For each set of data (i.e., the percentage of patients in which a particular sequence is up-regulated in a cancer tissue) the number of patients (Colon Cancer Patients; Colon Unmatched Met Patients and Colon Match Met Patients) is shown. If a sample is matched, it is matched to a sample from the same patient, if a sample is unmatched, the results obtained from that sample are compared to a pooled sample of an appropriate tissue type from the patients. If a sample is not from a metastasized tissue, it is from a primary tumor.

TABLE 25

| | | | Breast | | Colon | | Prostate |
|---|---|---|---|---|---|---|---|
| | | | Cancer | | Cancer | | Cancer |
| | | | Tumor/ | Breast | Tumor/ | Colon | Tumor/ |
| SEQ | | | Normal | Cancer | Normal | Cancer | Normal |
| ID | Seq Name | SpotID | >=2x | Patients | >=2x | Patients | >=2x |
| 138 | 3541.A16.GZ43_505167 | 58648 | | 23 | 26.09 | | |
| 151 | 3538.K12.GZ43_504729 | 56775 | | 23 | 26.00 | | |
| 181 | 3544.A17.GZ43_505567 | 56939 | | | | | |
| 205 | 3544.L13.GZ43_505514 | 60100 | | | | | 31.96 |
| 262 | 3550.D16.GZ43_506322 | 42108 | | | 44.74 | 76 | |
| 324 | 3553.J14.GZ43_506680 | 60233 | | | 47.37 | 19 | 37.11 |
| 331 | 3553.K03.GZ43_506505 | 55773 | 26.09 | 23 | 21.05 | 19 | 22.45 |
| 374 | 3556.C15.GZ43_507073 | 60100 | | | | | 31.96 |
| 392 | 3556.J14.GZ43_507064 | 60233 | | | 47.37 | 19 | 37.11 |
| 402 | 3556.M02.GZ43_506875 | 42108 | | | 44.74 | 76 | |
| 408 | 3556.N06.GZ43_506940 | 58440 | | | | | 20.41 |
| 467 | 3562.I02.GZ43_507639 | 58075 | | | | | 21.43 |
| 599 | 3574.F10.GZ43_509318 | 58075 | | | | | 21.43 |
| 603 | 3574.G11.GZ43_509335 | 56782 | 21.74 | 23 | | | |
| 605 | 3574.I02.GZ43_509193 | 60100 | | | | | 31.96 |
| 754 | 3590.D19.GZ43_512389 | 60100 | | | | | 31.96 |
| 768 | 3590.J01.GZ43_512107 | 60100 | | | | | 31.96 |
| 777 | 3590.L10.GZ43_512253 | 60100 | | | | | 31.96 |
| 792 | 3596.E08.GZ43_512598 | 60100 | | | | | 31.96 |
| 804 | 3596.K14.GZ43_512700 | 60100 | | | | | 31.96 |
| 814 | 3596.O10.GZ43_512640 | 60100 | | | | | 31.96 |
| 818 | 3596.P07.GZ43_512593 | 60100 | | | | | 31.96 |
| 841 | 3599.K23.GZ43_513228 | 57429 | | | | | 34.69 |
| 841 | 3599.K23.GZ43_513228 | 60100 | | | | | 31.96 |
| 846 | 3599.M24.GZ43_513246 | 35065 | | | 24.00 | 75 | |
| 851 | 3599.O06.GZ43_512960 | 60100 | | | | | 31.96 |
| 854 | 3602.A09.GZ43_513378 | 60100 | | | | | 31.96 |
| 873 | 3602.K06.GZ43_513340 | 60100 | | | | | 31.96 |
| 883 | 3605.I19.gz43_513930 | 56753 | | | | | 20.41 |
| 923 | 3611.I04.gz43_514458 | 60100 | | | | | 31.96 |
| 931 | 3611.L22.gz43_514749 | 60100 | | | | | 31.96 |
| 953 | 3614.P11.gz43_514961 | 56775 | 26.09 | 23 | | | |
| 955 | 3617.B16.gz43_515411 | 1368 | | | 34.29 | 35 | |
| 955 | 3617.B16.gz43_515411 | 25873 | | | 50.00 | 76 | |
| 955 | 3617.B16.gz43_515411 | 26658 | | | 59.21 | 76 | |
| 958 | 3617.H16.gz43_515417 | 59904 | | | | | 25.77 |
| 959 | 3617.I01.gz43_515178 | 57008 | | | | | 23.47 |
| 966 | 3617.N14.gz43_515391 | 57651 | | | | | 21.43 |
| 968 | 3617.P11.gz43_515345 | 56753 | | | | | 20.41 |
| 969 | 3617.P12.gz43_515361 | 60100 | | | | | 31.96 |
| 971 | 3620.B03.gz43_515810 | 57651 | | | | | 21.43 |
| 979 | 3620.G17.gz43_516039 | 60100 | | | | | 31.96 |
| 999 | 3623.N23.gz43_516526 | 27078 | | | 28.95 | 76 | |
| 1005 | 3626.G01.gz43_516551 | 33958 | 39.13 | 23 | | | 24.51 |
| 1005 | 3626.G01.gz43_516551 | 35113 | 39.13 | 23 | | | 23.53 |
| 1005 | 3626.G01.gz43_516551 | 58921 | 30.43 | 23 | | | 22.45 |
| 1009 | 3626.M15.gz43_516781 | 59829 | 26.09 | 23 | 36.84 | 19 | |
| 1030 | 3632.G01.gz43_517319 | 25933 | | | | | 39.22 |
| 1032 | 3632.K20.gz43_517627 | 60100 | | | | | 31.96 |
| 1034 | 3632.M13.gz43_517517 | 60100 | | | | | 31.96 |
| 1035 | 3632.M19.gz43_517613 | 57429 | | | | | 34.69 |
| 1035 | 3632.M19.gz43_517613 | 60100 | | | | | 31.96 |
| 1042 | 3635.A13.gz43_517889 | 60100 | | | | | 31.96 |
| 1063 | 3638.L10.gz43_518236 | 60100 | | | | | 31.96 |
| 1074 | 3643.I24.gz43_518841 | 59904 | | | | | 25.77 |
| 1117 | 3661.K22.gz43_519717 | 56753 | | | | | 20.41 |
| 1176 | 3664.K19.gz43_520821 | 25933 | | | | | 39.22 |
| 1179 | 3664.P12.gz43_520714 | 57008 | | | | | 23.47 |
| 1179 | 3664.P12.gz43_520714 | 57797 | | | | | 21.43 |
| 1214 | 3666.L23.gz43_521654 | 53114 | | | | | 26.47 |
| 1224 | 3754.B08.gz43_532950 | 60100 | | | | | 31.96 |
| 1257 | 3756.A02.gz43_533237 | 60100 | | | | | 31.96 |
| 1266 | 3756.C16.gz43_533463 | 60100 | | | | | 31.96 |
| 1272 | 3756.E12.gz43_533401 | 57651 | | | | | 21.43 |
| 1278 | 3756.G14.gz43_533435 | 60100 | | | | | 31.96 |
| 1306 | 3759.K05.gz43_533679 | 59904 | | | | | 25.77 |
| 1325 | 3762.A20.gz43_534293 | 60233 | | | 47.37 | 19 | 37.11 |
| 1348 | 3762.L18.gz43_534272 | 60100 | | | | | 31.96 |
| 1354 | Clu8293.con_1 | 60100 | | | | | 31.96 |
| 1372 | Clu403488.con_1 | 60100 | | | | | 31.96 |
| 1409 | Clu609914.con_1 | 24511 | | | | | |
| 1411 | Clu621702.con_1 | 35065 | | | 24.00 | 75 | |
| 1427 | Clu733840.con_1 | 24511 | | | | | |
| 1435 | Clu777670.con_1 | 60100 | | | | | 31.96 |
| 1441 | Clu854573.con_1 | 57429 | | | | | 34.69 |
| 1441 | Clu854573.con_1 | 60100 | | | | | 31.96 |
| 1459 | Clu1053799.con_1 | 58075 | | | | | 21.43 |
| 1465 | Clu1054813.con_1 | 60233 | | | 47.37 | 19 | 37.11 |
| 1471 | Clu1055326.con_1 | 25933 | | | | | 39.22 |
| 1492 | Clu1088930.con_1 | 27078 | | | 28.95 | 76 | |
| 1522 | Clu1224379.con_1 | 42108 | | | 44.74 | 76 | |
| 1541 | Clu1228277.con_1 | 60100 | | | | | 31.96 |
| 1546 | Clu1259069.con_2 | 25844 | | | | | 51.96 |
| 1546 | Clu1259069.con_2 | 28996 | | | | | 49.02 |
| 1549 | Clu1292262.con_1 | 56753 | | | | | 20.41 |
| 1554 | Clu1292436.con_1 | 59904 | | | | | 25.77 |
| 1573 | NTN_007592S2.3_10 | 53100 | | | 25.33 | 75 | |
| 1584 | NTN_009296S1.3_1 | 1368 | | | 34.29 | 35 | |
| 1584 | NTN_009296S1.3_1 | 25873 | | | 50.00 | 76 | |
| 1584 | NTN_009296S1.3_1 | 26658 | | | 59.21 | 76 | |
| 1585 | NTN_009296S3.3_2 | 56753 | | | | | 20.41 |
| 1600 | NTN_011512S51.3_3 | 35086 | | | 22.67 | 75 | |
| 1600 | NTN_011512S51.3_3 | 36824 | | | 21.33 | 75 | |
| 1601 | NTN_017582S2.3_6 | 26345 | | | 69.74 | 76 | |
| 1615 | NTN_025842S13.2_1 | 27078 | | | 28.95 | 76 | |

| | | | | | | Colon | |
|---|---|---|---|---|---|---|---|
| | | Colon | Colon | Colon | Colon | Matched | Colon |
| | Prostate | Unmatched | Unmatched | Matched | Matched | Met/ | Match |
| SEQ | Cancer | Met/Normal | Met | Met/Normal | Met | Tumor | Met |
| ID | Patients | >=2x | Patients | >=2x | Patients | >=2x | Patients |
| 138 | | | | | | | |
| 151 | | | | | | | |
| 181 | | | | | | 22.22 | 18 |
| 205 | 97 | | | | | | |
| 262 | | 63.64 | 33 | 52.78 | 36 | | |
| 324 | 97 | | | 41.18 | 17 | | |
| 331 | 98 | | | | | | |
| 374 | 97 | | | | | | |
| 392 | 97 | | | 41.18 | 17 | | |
| 402 | | 63.64 | 33 | 52.78 | 36 | | |
| 408 | 98 | | | | | | |
| 467 | 98 | | | | | | |
| 599 | 98 | | | | | | |
| 603 | | | | | | | |
| 605 | 97 | | | | | | |
| 754 | 97 | | | | | | |
| 768 | 97 | | | | | | |
| 777 | 97 | | | | | | |
| 792 | 97 | | | | | | |
| 804 | 97 | | | | | | |
| 814 | 97 | | | | | | |
| 818 | 97 | | | | | | |
| 841 | 98 | | | | | | |
| 841 | 97 | | | | | | |
| 846 | | 21.21 | 33 | | | | |
| 851 | 97 | | | | | | |
| 854 | 97 | | | | | | |
| 873 | 97 | | | | | | |
| 883 | 98 | | | | | | |
| 923 | 97 | | | | | | |
| 931 | 97 | | | | | | |
| 953 | | | | | | | |
| 955 | | 56.67 | 30 | 28.57 | 7 | | |
| 955 | | 57.58 | 33 | 55.56 | 36 | | |
| 955 | | 66.67 | 33 | 44.44 | 36 | | |
| 958 | 97 | | | | | | |
| 959 | 98 | | | | | | |
| 966 | 98 | | | | | | |
| 968 | 98 | | | | | | |
| 969 | 97 | | | | | | |
| 971 | 98 | | | | | | |
| 979 | 97 | | | | | | |
| 999 | | 15.15 | 33 | | | | |
| 1005 | 102 | 18.18 | 33 | | | | |
| 1005 | 102 | 15.15 | 33 | | | | |
| 1005 | 98 | | | | | | |
| 1009 | | | | 47.06 | 17 | | |
| 1030 | 102 | 0.00 | 33 | | | | |
| 1032 | 97 | | | | | | |
| 1034 | 97 | | | | | | |
| 1035 | 98 | | | | | | |
| 1035 | 97 | | | | | | |
| 1042 | 97 | | | | | | |
| 1063 | 97 | | | | | | |
| 1074 | 97 | | | | | | |
| 1117 | 98 | | | | | | |
| 1176 | 102 | 0.00 | 33 | | | | |
| 1179 | 98 | | | | | | |
| 1179 | 98 | | | | | | |
| 1214 | 102 | 0.00 | 33 | | | | |
| 1224 | 97 | | | | | | |
| 1257 | 97 | | | | | | |
| 1266 | 97 | | | | | | |
| 1272 | 98 | | | | | | |
| 1278 | 97 | | | | | | |
| 1306 | 97 | | | | | | |
| 1325 | 97 | | | 41.18 | 17 | | |
| 1348 | 97 | | | | | | |
| 1354 | 97 | | | | | | |
| 1372 | 97 | | | | | | |
| 1409 | | 24.24 | 33 | 26.09 | 23 | | |
| 1411 | | 21.21 | 33 | | | | |
| 1427 | | 24.24 | 33 | 26.09 | 23 | | |
| 1435 | 97 | | | | | | |
| 1441 | 98 | | | | | | |
| 1441 | 97 | | | | | | |
| 1459 | 98 | | | | | | |
| 1465 | 97 | | | 41.18 | 17 | | |
| 1471 | 102 | 0.00 | 33 | | | | |
| 1492 | | 15.15 | 33 | | | | |
| 1522 | | 63.64 | 33 | 52.78 | 36 | | |
| 1541 | 97 | | | | | | |
| 1546 | 102 | 0.00 | 33 | | | | |
| 1546 | 102 | 0.00 | 33 | | | | |
| 1549 | 98 | | | | | | |
| 1554 | 97 | | | | | | |
| 1573 | | 6.06 | 33 | 28.57 | 35 | | |
| 1584 | | 56.67 | 30 | 28.57 | 7 | | |
| 1584 | | 57.58 | 33 | 55.56 | 36 | | |
| 1584 | | 66.67 | 33 | 44.44 | 36 | | |
| 1585 | 98 | | | | | | |
| 1600 | | 9.09 | 33 | 25.00 | 36 | | |
| 1600 | | 12.12 | 33 | 33.33 | 36 | | |
| 1601 | | 78.79 | 33 | 66.67 | 36 | | |
| 1615 | | 15.15 | 33 | | | | |

These data provide evidence that the genes represented by the polynucleotides having the indicated sequences are differentially expressed in breast, prostate, cancer as compared to normal non-cancerous breast tissue and are differentially expressed in colon cancer as compared to normal non-cancerous colon tissue

The above methods can be performed to identify genes differentially expressed in cancerous and normal cells of any type of tissue, such as prostate, lung, colon, breast, and the like.

### Example 23

### Antisense Regulation of Gene Expression

The expression of the differentially expressed genes represented by the polynucleotides in the cancerous cells can be further analyzed using antisense knockout technology to confirm the role and function of the gene product in tumorigenesis, e.g., in promoting a metastatic phenotype.

Methods for analysis using antisense technology are well known in the art. For example, a number of different oligonucleotides complementary to the mRNA generated by the differentially expressed genes identified herein can be designed as antisense oligonucleotides, and tested for their ability to suppress expression of the genes. Sets of antisense oligomers specific to each candidate target are designed using the sequences of the polynucleotides corresponding to a differentially expressed gene and the software program HYBsimulator Version 4 (available for Windows 95/Windows NT or for Power Macintosh, RNAture, Inc. 1003 Health Sciences Road, West, Irvine, Calif. 92612 USA). Factors considered when designing antisense oligonucleotides include: 1) the The expression of the differentially expressed genes represented by the polynucleotides in the cancerous cells can be analyzed using antisense knockout technology to confirm the role and function of the gene product in tumorigenesis, e.g., in promoting a metastatic phenotype.

A number of different oligonucleotides complementary to the mRNA generated by the differentially expressed genes identified herein can be designed as potential antisense oligonucleotides, and tested for their ability to suppress expression of the genes. Sets of antisense oligomers specific to each candidate target are designed using the sequences of the polynucleotides corresponding to a differentially expressed gene and the software program HYBsimulator Version 4 (available for Windows 95/Windows NT or for Power Macintosh, RNAture, Inc. 1003 Health Sciences Road, West, Irvine, Calif. 92612 USA). Factors that are considered when designing antisense oligonucleotides include: 1) the secondary structure of oligonucleotides; 2) the secondary structure of the target gene; 3) the specificity with no or minimum cross-hybridization to other expressed genes; 4) stability; 5) length and 6) terminal GC content. The antisense oligonucleotide is designed so that it will hybridize to its target sequence under conditions of high stringency at physiological temperatures (e.g., an optimal temperature for the cells in culture to provide for hybridization in the cell, e.g., about 37° C.), but with minimal formation of homodimers.

Using the sets of oligomers and the HYBsimulator program, three to ten antisense oligonucleotides and their reverse controls are designed and synthesized for each candidate mRNA transcript, which transcript is obtained from the gene corresponding to the target polynucleotide sequence of interest. Once synthesized and quantitated, the oligomers are screened for efficiency of a transcript knock-out in a panel of cancer cell lines. The efficiency of the knock-out is determined by analyzing mRNA levels using lightcycler quantification. The oligomers that resulted in the highest level of transcript knock-out, wherein the level was at least about 50%, preferably about 80-90%, up to 95% or more up to undetectable message, are selected for use in a cell-based proliferation assay, an anchorage independent growth assay, and an apoptosis assay.

The ability of each designed antisense oligonucleotide to inhibit gene expression is tested through transfection into LNCaP, PC3, 22Rv1, MDA-PCA-2b, or DU145 prostate carcinoma cells. For each transfection mixture, a carrier molecule (such as a lipid, lipid derivative, lipid-like molecule, cholesterol, cholesterol derivative, or cholesterol-like molecule) is prepared to a working concentration of 0.5 mM in water, sonicated to yield a uniform solution, and filtered through a 0.45 μm PVDF membrane. The antisense or control oligonucleotide is then prepared to a working concentration of 100 μM in sterile Millipore water. The oligonucleotide is further diluted in OptiMEM™ (Gibco/BRL), in a microfuge tube, to 2 μM, or approximately 20 μg oligo/ml of OptiMEM™. In a separate microfuge tube, the carrier molecule, typically in the amount of about 1.5-2 nmol carrier/μg antisense oligonucleotide, is diluted into the same volume of OptiMEM™ used to dilute the oligonucleotide. The diluted antisense oligonucleotide is immediately added to the diluted carrier and mixed by pipetting up and down. Oligonucleotide is added to the cells to a final concentration of 30 nM.

The level of target mRNA that corresponds to a target gene of interest in the transfected cells is quantitated in the cancer cell lines using the Roche LightCycler™ real-time PCR machine. Values for the target mRNA are normalized versus an internal control (e.g., beta-actin). For each 20 μl reaction, extracted RNA (generally 0.2-1 μg total) is placed into a sterile 0.5 or 1.5 ml microcentrifuge tube, and water is added to a total volume of 12.5 μl. To each tube is added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin® (20 u) (Ambion, Inc., Hialeah, Fla.), and 0.5 μl MMLV reverse transcriptase (50 u) (Ambion, Inc.). The contents are mixed by pipetting up and down, and the reaction mixture is incubated at 42° C. for 1 hour. The contents of each tube are centrifuged prior to amplification.

An amplification mixture is prepared by mixing in the following order: 1× PCR buffer 11, 3 mM MgCl₂, 140 μM each dNTP, 0.175 pmol each oligo, 1:50,000 dil of SYBR® Green, 0.25 mg/ml BSA, 1 unit Taq polymerase, and H₂₀ to 20 μl. (PCR buffer II is available in 10× concentration from Perkin-Elmer, Norwalk, Conn.). In 1× concentration it contains 10 mM Tris pH 8.3 and 50 mM KCl. SYBR® Green (Molecular Probes, Eugene, Oreg.) is a dye which fluoresces when bound to double stranded DNA. As double stranded PCR product is produced during amplification, the fluorescence from SYBR® Green increases. To each 20 μl aliquot of amplification mixture, 2 μl of template RT is added, and amplification is carried out according to standard protocols. The results are expressed as the percent decrease in expression of the corresponding gene product relative to non-transfected cells, vehicle-only transfected (mock-transfected) cells, or cells transfected with reverse control oligonucleotides.

### Example 24

### Effect of Expression on Proliferation

The effect of gene expression on the inhibition of cell proliferation can be assessed in metastatic breast cancer cell lines (MDA-MB-231 (“231”)); SW620 colon colorectal carcinoma cells; SKOV3 cells (a human ovarian carcinoma cell line); or LNCaP, PC3, 22Rv1, MDA-PCA-2b, or DU145 prostate cancer cells.

Cells are plated to approximately 60-80% confluency in 96-well dishes. Antisense or reverse control oligonucleotide is diluted to 2 μM in OptiMEM™. The oligonucleotide-OptiMEM™ can then be added to a delivery vehicle, which delivery vehicle can be selected so as to be optimized for the particular cell type to be used in the assay. The oligo/delivery vehicle mixture is then further diluted into medium with serum on the cells. The final concentration of oligonucleotide for all experiments can be about 300 nM.

Antisense oligonucleotides are prepared as described above (see Example 3). Cells are transfected overnight at 37° C. and the transfection mixture is replaced with fresh medium the next morning. Transfection is carried out as described above in Example 23.

Those antisense oligonucleotides that result in inhibition of proliferation of SW620 cells indicate that the corresponding gene plays a role in production or maintenance of the cancerous phenotype in cancerous colon cells. Those antisense oligonucleotides that inhibit proliferation in SKOV3 cells represent genes that play a role in production or maintenance of the cancerous phenotype in cancerous breast cells. Those antisense oligonucleotides that result in inhibition of proliferation of MDA-MB-231 cells indicate that the corresponding gene plays a role in production or maintenance of the cancerous phenotype in cancerous ovarian cells. Those antisense oligonucleotides that inhibit proliferation in LNCaP, PC3, 22Rv1, MDA-PCA-2b, or DU145 cells represent genes that play a role in production or maintenance of the cancerous phenotype in cancerous prostate cells.

Using the following antisense oligonucleotides: TTGGTTCCCAAGACAAGCCGTGAC (SEQ ID NO:1676); TCTCAACGCTACCAGGCACTCCTTG (SEQ ID NO:1677); GCACAGCCCAAAGTCAAAGGCATTA (SEQ ID NO:1678); CAGGCACTCCTTGGTCAAATGTGGG (SEQ ID NO:1679); GGACAGGGAAAGGAGAGGCTAGTCA (SEQ ID NO:1680) and TGCATTCTCTCCCACATCTCAACGC SEQ ID NO:1681, corresponding to a glutothione transferase omega identified by SEQ ID NOS: 1510 and 1674 (Chiron Candidate Id 21), were used to inhibit proliferation of SW620 colon colorectal carcinoma cells. These antisense molecules reduced glutothione transferase omega RNA expression by approximately 90%.

### Example 25

### Effect of Gene Expression on Cell Migration

The effect of gene expression on the inhibition of cell migration can be assessed in LNCaP, PC3, 22Rv1, MDA-PCA-2b, or DU145 prostate cancer cells using static endothelial cell binding assays, non-static endothelial cell binding assays, and transmigration assays.

For the static endothelial cell binding assay, antisense oligonucleotides are prepared as described above (see Example 23). Two days prior to use, prostate cancer cells (CaP) are plated and transfected with antisense oligonucleotide as described above (see above). On the day before use, the medium is replaced with fresh medium, and on the day of use, the medium is replaced with fresh medium containing 2 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in DMEM/1% BSA/10 mM HEPES pH 7.0. Finally, CaP cells are counted and resuspended at a concentration of 1×10⁶ cells/ml.

Endothelial cells (EC) are plated onto 96-well plates at 40-50% confluence 3 days prior to use. On the day of use, EC are washed 1× with PBS and 50λ DMDM/1% BSA/10 mM HEPES pH 7 is added to each well. To each well is then added 50K (50λ) CaP cells in DMEM/1% BSA/10 mM HEPES pH 7. The plates are incubated for an additional 30 min and washed 5× with PBS containing Ca⁺⁺ and Mg⁺⁺. After the final wash, 100 μL PBS is added to each well and fluorescence is read on a fluorescent plate reader (Ab492/Em 516 nm).

For the non-static endothelial cell binding assay, CaP are prepared as described above. EC are plated onto 24-well plates at 30-40% confluence 3 days prior to use. On the day of use, a subset of EC are treated with cytokine for 6 hours then washed 2× with PBS. To each well is then added 150-200K CaP cells in DMEM/1% BSA/10 mM HEPES pH 7. Plates are placed on a rotating shaker (70 RPM) for 30 min and then washed 3× with PBS containing Ca⁺⁺ and Mg⁺⁺. After the final wash, 500 μL PBS is added to each well and fluorescence is read on a fluorescent plate reader (Ab492/Em 516 nm).

For the transmigration assay, CaP are prepared as described above with the following changes. On the day of use, CaP medium is replaced with fresh medium containing 5 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in EGM-2-MV medium. Finally, CaP cells are counted and resuspended at a concentration of 1×10⁶ cells/ml.

EC are plated onto FluorBlok transwells (BD Biosciences) at 30-40% confluence 5-7 days before use. Medium is replaced with fresh medium 3 days before use and on the day of use. To each transwell is then added 50K labeled CaP. 30 min prior to the first fluorescence reading, 10 μg of FITC-dextran (10K MW) is added to the EC plated filter. Fluorescence is then read at multiple time points on a fluorescent plate reader (Ab492/Em 516 nm).

Those antisense oligonucleotides that result in inhibition of binding of LNCaP, PC3, 22Rv1, MDA-PCA-2b, or DU145 prostate cancer cells to endothelial cells indicate that the corresponding gene plays a role in the production or maintenance of the cancerous phenotype in cancerous prostate cells. Those antisense oligonucleotides that result in inhibition of endothelial cell transmigration by LNCaP, PC3, 22Rv1, MDA-PCA-2b, or DU145 prostate cancer cells indicate that the corresponding gene plays a role in the production or maintenance of the cancerous phenotype in cancerous prostate cells.

### Example 26

### Effect of Gene Expression on Colony Formation

The effect of gene expression upon colony formation of SW620 cells, SKOV3 cells, MD-MBA-231 cells, LNCaP cells, PC3 cells, 22Rv1 cells, MDA-PCA-2b cells, and DU145 cells can be tested in a soft agar assay. Soft agar assays are conducted by first establishing a bottom layer of 2 ml of 0.6% agar in media plated fresh within a few hours of layering on the cells. The cell layer is formed on the bottom layer by removing cells transfected as described above from plates using 0.05% trypsin and washing twice in media. The cells are counted in a Coulter counter, and resuspended to 10⁶ per ml in media. 10 μl aliquots are placed with media in 96-well plates (to check counting with WST1), or diluted further for the soft agar assay. 2000 cells are plated in 800 μl 0.4% agar in duplicate wells above 0.6% agar bottom layer. After the cell layer agar solidifies, 2 ml of media is dribbled on top and antisense or reverse control oligo (produced as described in above) is added without delivery vehicles. Fresh media and oligos are added every 3-4 days. Colonies form in 10 days to 3 weeks. Fields of colonies are counted by eye. Wst-1 metabolism values can be used to compensate for small differences in starting cell number. Larger fields can be scanned for visual record of differences.

Those antisense oligonucleotides that result in inhibition of colony formation of SW620 cells indicate that the corresponding gene plays a role in production or maintenance of the cancerous phenotype in cancerous colon cells. Those antisense oligonucleotides that inhibit colony formation in SKOV3 cells represent genes that play a role in production or maintenance of the cancerous phenotype in cancerous breast cells. Those antisense oligonucleotides that result in inhibition of colony formation of MDA-MB-231 cells indicate that the corresponding gene plays a role in production or maintenance of the cancerous phenotype in cancerous ovarian cells. Those antisense oligonucleotides that inhibit colony formation in LNCaP, PC3, 22Rv1, MDA-PCA-2b, or DU145 cells represent genes that play a role in production or maintenance of the cancerous phenotype in cancerous prostate cells.

### Example 27

### Induction of Cell Death Upon Depletion of Polypeptides by Depletion of mRNA (“Antisense Knockout”)

In order to assess the effect of depletion of a target message upon cell death, LNCaP, PC3, 22Rv1, MDA-PCA-2b, or DU145 cells, or other cells derived from a cancer of interest, can be transfected for proliferation assays. For cytotoxic effect in the presence of cisplatin (cis), the same protocol is followed but cells are left in the presence of 2 μM drug. Each day, cytotoxicity is monitored by measuring the amount of LDH enzyme released in the medium due to membrane damage. The activity of LDH is measured using the Cytotoxicity Detection Kit from Roche Molecular Biochemicals. The data is provided as a ratio of LDH released in the medium vs. the total LDH present in the well at the same time point and treatment (rLDH/tLDH). A positive control using antisense and reverse control oligonucleotides for BCL2 (a known anti-apoptotic gene) is included; loss of message for BCL2 leads to an increase in cell death compared with treatment with the control oligonucleotide (background cytotoxicity due to transfection).

### Example 28

### Functional Analysis of Gene Products Differentially Expressed in Cancer

The gene products of sequences of a gene differentially expressed in cancerous cells can be further analyzed to confirm the role and function of the gene product in tumorigenesis, e.g., in promoting or inhibiting development of a metastatic phenotype. For example, the function of gene products corresponding to genes identified herein can be assessed by blocking function of the gene products in the cell. For example, where the gene product is secreted or associated with a cell surface membrane, blocking antibodies can be generated and added to cells to examine the effect upon the cell phenotype in the context of, for example, the transformation of the cell to a cancerous, particularly a metastatic, phenotype. In order to generate antibodies, a clone corresponding to a selected gene product is selected, and a sequence that represents a partial or complete coding sequence is obtained. The resulting clone is expressed, the polypeptide produced isolated, and antibodies generated. The antibodies are then combined with cells and the effect upon tumorigenesis assessed.

Where the gene product of the differentially expressed genes identified herein exhibits sequence homology to a protein of known function (e.g., to a specific kinase or protease) and/or to a protein family of known function (e.g., contains a domain or other consensus sequence present in a protease family or in a kinase family), then the role of the gene product in tumorigenesis, as well as the activity of the gene product, can be examined using small molecules that inhibit or enhance function of the corresponding protein or protein family.

Additional functional assays include, but are not necessarily limited to, those that analyze the effect of expression of the corresponding gene upon cell cycle and cell migration. Methods for performing such assays are well known in the art.

### Example 29

### Deposit Information

Deposits of the biological materials in the tables referenced below were made with either the Agricultural Research Service Culture Collection (NRRL), 1815 North University Street, Peoria, Ill. 61604, or with the American Type Culture Collection (ATCC), 10801 University Blvd., Manasas, Va. 20110-2209, under the provisions of the Budapest Treaty, on or before the filing date of the present application. The accession number indicated is assigned after successful viability testing, and the requisite fees were paid. Access to said cultures will be available during pendency of the patent application to one determined by the Commissioner to be entitled to such under 37 C.F.R. § 1.14 and 35 U.S.C. §122. All restriction on availability of said cultures to the public will be irrevocably removed upon the granting of a patent based upon the application. Moreover, the designated deposits will be maintained for a period of thirty (30) years from the date of deposit, or for five (5) years after the last request for the deposit; or for the enforceable life of the U.S. patent, whichever is longer. Should a culture become nonviable or be inadvertently destroyed, or, in the case of plasmid-containing strains, lose its plasmid, it will be replaced with a viable culture(s) of the same taxonomic description.

These deposits are provided merely as a convenience to those of skill in the art, and are not an admission that a deposit is required. A license may be required to make, use, or sell the deposited materials, and no such license is hereby granted. The deposit below was received by the ATCC on or before the filing date of the present application.

TABLE 23
Cell Lines Deposited with ATCC

| | | ATCC Accession | CMCC Accession |
|---|---|---|---|
| Cell Line | Deposit Date | No. | No. |
| KM12L4-A | Mar. 19, 1998 | CRL-12496 | 11606 |
| Km12C | May 15, 1998 | CRL-12533 | 11611 |
| MDA-MB- | May 15, 1998 | CRL-12532 | 10583 |
| 231 | | | |
| MCF-7 | Oct. 9, 1998 | CRL-12584 | 10377 |

In addition, pools of selected clones, as well as libraries containing specific clones, were assigned an “ES” number and a “CMCC” number (both internal references) and deposited with the NRRL. Table 24 provides the NRRL Accession Nos. of the clones deposited as librarires named ES219-ES225 (CMCC5471-CMCC5477, respectively) on Nov. 1, 2001, and of the clones deposited as a library named ES226 (CMCC5478) on Nov. 7, 2001.

TABLE 24

| Library | CMCC | | NRRL |
|---|---|---|---|
| ID | Number | CloneId | Number |
| ES219 | 5471 | M00084879B:E01 | B-30523 |
| ES219 | 5471 | M00083819B:E10 | B-30523 |
| ES219 | 5471 | M00084942C:B10 | B-30523 |
| ES219 | 5471 | M00084704C:B09 | B-30523 |
| ES219 | 5471 | M00084887C:C07 | B-30523 |
| ES219 | 5471 | M00084976B:A08 | B-30523 |
| ES219 | 5471 | M00085011B:A01 | B-30523 |
| ES219 | 5471 | M00084961A:C07 | B-30523 |
| ES219 | 5471 | M00084960D:D02 | B-30523 |
| ES219 | 5471 | M00084973A:B06 | B-30523 |
| ES219 | 5471 | M00084928D:F06 | B-30523 |
| ES219 | 5471 | M00084968C:D10 | B-30523 |
| ES219 | 5471 | M00084973A:B06 | B-30523 |
| ES219 | 5471 | M00084966A:A08 | B-30523 |
| ES219 | 5471 | M00084919C:B04 | B-30523 |
| ES219 | 5471 | M00085003C:D03 | B-30523 |
| ES219 | 5471 | M00084968A:D01 | B-30523 |
| ES219 | 5471 | M00084969D:C11 | B-30523 |
| ES219 | 5471 | M00084899D:B01 | B-30523 |
| ES219 | 5471 | M00084893C:A12 | B-30523 |
| ES219 | 5471 | M00084890D:F09 | B-30523 |
| ES219 | 5471 | M00084904A:D03 | B-30523 |
| ES219 | 5471 | M00085029A:C02 | B-30523 |
| ES219 | 5471 | M00084963D:D07 | B-30523 |
| ES219 | 5471 | M00085147C:A04 | B-30523 |
| ES219 | 5471 | M00085144B:C12 | B-30523 |
| ES219 | 5471 | M00085124B:G05 | B-30523 |
| ES219 | 5471 | M00085702B:G11 | B-30523 |
| ES219 | 5471 | M00085203A:E06 | B-30523 |
| ES219 | 5471 | M00085242A:C06 | B-30523 |
| ES219 | 5471 | M00084980D:H08 | B-30523 |
| ES219 | 5471 | M00085187B:C11 | B-30523 |
| ES219 | 5471 | M00085021C:F06 | B-30523 |
| ES219 | 5471 | M00085182B:E04 | B-30523 |
| ES219 | 5471 | M00084930D:B08 | B-30523 |
| ES219 | 5471 | M00084941B:E07 | B-30523 |
| ES219 | 5471 | M00084424D:G07 | B-30523 |
| ES219 | 5471 | M00084938B:F12 | B-30523 |
| ES219 | 5471 | M00084853D:G03 | B-30523 |
| ES219 | 5471 | M00084878B:B12 | B-30523 |
| ES219 | 5471 | M00084889B:C02 | B-30523 |
| ES219 | 5471 | M00084885D:A12 | B-30523 |
| ES219 | 5471 | M00084845A:E02 | B-30523 |
| ES219 | 5471 | M00084972B:H03 | B-30523 |
| ES219 | 5471 | M00084908A:F03 | B-30523 |
| ES219 | 5471 | M00084975A:G05 | B-30523 |
| ES219 | 5471 | M00084941C:H04 | B-30523 |
| ES219 | 5471 | M00084997D:H09 | B-30523 |
| ES219 | 5471 | M00084491A:E08 | B-30523 |
| ES219 | 5471 | M00083815C:H08 | B-30523 |
| ES219 | 5471 | M00084501A:D06 | B-30523 |
| ES219 | 5471 | M00084558D:G08 | B-30523 |
| ES219 | 5471 | M00084510C:F02 | B-30523 |
| ES219 | 5471 | M00084521C:H11 | B-30523 |
| ES219 | 5471 | M00084446A:A05 | B-30523 |
| ES219 | 5471 | M00084458A:G06 | B-30523 |
| ES219 | 5471 | M00084377D:E08 | B-30523 |
| ES219 | 5471 | M00084382A:D06 | B-30523 |
| ES219 | 5471 | M00083816B:D08 | B-30523 |
| ES219 | 5471 | M00084449B:C09 | B-30523 |
| ES219 | 5471 | M00084431C:B02 | B-30523 |
| ES219 | 5471 | M00084463A:B07 | B-30523 |
| ES219 | 5471 | M00084487D:F04 | B-30523 |
| ES219 | 5471 | M00083800C:E07 | B-30523 |
| ES219 | 5471 | M00084468C:E07 | B-30523 |
| ES219 | 5471 | M00084638A:E10 | B-30523 |
| ES219 | 5471 | M00084439B:A08 | B-30523 |
| ES219 | 5471 | M00084479D:E10 | B-30523 |
| ES219 | 5471 | M00084455D:B03 | B-30523 |
| ES219 | 5471 | M00084368D:C02 | B-30523 |
| ES219 | 5471 | M00084642D:E08 | B-30523 |
| ES219 | 5471 | M00084373A:F08 | B-30523 |
| ES219 | 5471 | M00084364C:B06 | B-30523 |
| ES219 | 5471 | M00084521B:E11 | B-30523 |
| ES219 | 5471 | M00084385B:D03 | B-30523 |
| ES219 | 5471 | M00084443C:H06 | B-30523 |
| ES219 | 5471 | M00083803C:F03 | B-30523 |
| ES219 | 5471 | M00084421C:B11 | B-30523 |
| ES219 | 5471 | M00084434B:E06 | B-30523 |
| ES219 | 5471 | M00083820B:C03 | B-30523 |
| ES219 | 5471 | M00084246B:H03 | B-30523 |
| ES219 | 5471 | M00084484C:B11 | B-30523 |
| ES219 | 5471 | M00084410C:F10 | B-30523 |
| ES219 | 5471 | M00083801B:H03 | B-30523 |
| ES219 | 5471 | M00084980C:B07 | B-30523 |
| ES219 | 5471 | M00084499C:C11 | B-30523 |
| ES219 | 5471 | M00084526C:G09 | B-30523 |
| ES219 | 5471 | M00084406C:A01 | B-30523 |
| ES219 | 5471 | M00084380D:B07 | B-30523 |
| ES219 | 5471 | M00084383B:A11 | B-30523 |
| ES219 | 5471 | M00083834C:E02 | B-30523 |
| ES219 | 5471 | M00083839A:H03 | B-30523 |
| ES219 | 5471 | M00084505C:H08 | B-30523 |
| ES219 | 5471 | M00084511D:A02 | B-30523 |
| ES219 | 5471 | M00084494C:C01 | B-30523 |
| ES219 | 5471 | M00084451D:F06 | B-30523 |
| ES219 | 5471 | M00084604A:D02 | B-30523 |
| ES219 | 5471 | M00084771D:G03 | B-30523 |
| ES219 | 5471 | M00084817A:H11 | B-30523 |
| ES219 | 5471 | M00084827D:D04 | B-30523 |
| ES219 | 5471 | M00084843D:C06 | B-30523 |
| ES219 | 5471 | M00084750C:B08 | B-30523 |
| ES219 | 5471 | M00084757A:D01 | B-30523 |
| ES219 | 5471 | M00084771D:A01 | B-30523 |
| ES219 | 5471 | M00084730B:A09 | B-30523 |
| ES219 | 5471 | M00084826B:E11 | B-30523 |
| ES219 | 5471 | M00084595C:C07 | B-30523 |
| ES219 | 5471 | M00084724A:C02 | B-30523 |
| ES219 | 5471 | M00084833A:G07 | B-30523 |
| ES219 | 5471 | M00084600D:B10 | B-30523 |
| ES219 | 5471 | M00084634C:H02 | B-30523 |
| ES219 | 5471 | M00084614D:A08 | B-30523 |
| ES219 | 5471 | M00084620B:F05 | B-30523 |
| ES219 | 5471 | M00084607A:B03 | B-30523 |
| ES219 | 5471 | M00084633A:B12 | B-30523 |
| ES219 | 5471 | M00084597A:F06 | B-30523 |
| ES219 | 5471 | M00084575A:A11 | B-30523 |
| ES219 | 5471 | M00084547B:B10 | B-30523 |
| ES219 | 5471 | M00084525A:E08 | B-30523 |
| ES219 | 5471 | M00084578B:E12 | B-30523 |
| ES219 | 5471 | M00084669A:A05 | B-30523 |
| ES219 | 5471 | M00084419C:A09 | B-30523 |
| ES219 | 5471 | M00084769C:H03 | B-30523 |
| ES219 | 5471 | M00085007A:B03 | B-30523 |
| ES219 | 5471 | M00084865D:B04 | B-30523 |
| ES219 | 5471 | M00084743D:G01 | B-30523 |
| ES219 | 5471 | M00084770B:G12 | B-30523 |
| ES219 | 5471 | M00084584B:A02 | B-30523 |
| ES219 | 5471 | M00084647C:E12 | B-30523 |
| ES219 | 5471 | M00084766D:F12 | B-30523 |
| ES219 | 5471 | M00084648D:F05 | B-30523 |
| ES219 | 5471 | M00084843A:D06 | B-30523 |
| ES219 | 5471 | M00084709C:B02 | B-30523 |
| ES219 | 5471 | M00084834B:G02 | B-30523 |
| ES219 | 5471 | M00084718D:C04 | B-30523 |
| ES219 | 5471 | M00084702B:C12 | B-30523 |
| ES219 | 5471 | M00084645D:G02 | B-30523 |
| ES219 | 5471 | M00084849B:F11 | B-30523 |
| ES219 | 5471 | M00084859C:H05 | B-30523 |
| ES219 | 5471 | M00084850D:H02 | B-30523 |
| ES219 | 5471 | M00084857B:A09 | B-30523 |
| ES219 | 5471 | M00084867A:C11 | B-30523 |
| ES219 | 5471 | M00084823A:H01 | B-30523 |
| ES219 | 5471 | M00084756B:H01 | B-30523 |
| ES219 | 5471 | M00084700D:E09 | B-30523 |
| ES219 | 5471 | M00085010C:H01 | B-30523 |
| ES219 | 5471 | M00085060B:C05 | B-30523 |
| ES219 | 5471 | M00085012C:A08 | B-30523 |
| ES219 | 5471 | M00085047D:F03 | B-30523 |
| ES219 | 5471 | M00085049B:E03 | B-30523 |
| ES219 | 5471 | M00085051C:A01 | B-30523 |
| ES219 | 5471 | M00085050A:E11 | B-30523 |
| ES219 | 5471 | M00085676C:C04 | B-30523 |
| ES219 | 5471 | M00085121A:D10 | B-30523 |
| ES219 | 5471 | M00085166D:C10 | B-30523 |
| ES219 | 5471 | M00084992D:B02 | B-30523 |
| ES219 | 5471 | M00085148B:H01 | B-30523 |
| ES219 | 5471 | M00085123B:C04 | B-30523 |
| ES219 | 5471 | M00085173B:A08 | B-30523 |
| ES219 | 5471 | M00085172C:F06 | B-30523 |
| ES219 | 5471 | M00084937D:B04 | B-30523 |
| ES219 | 5471 | M00085026D:A01 | B-30523 |
| ES219 | 5471 | M00084994D:F11 | B-30523 |
| ES219 | 5471 | M00085190C:D10 | B-30523 |
| ES219 | 5471 | M00085194D:F04 | B-30523 |
| ES219 | 5471 | M00085222D:D07 | B-30523 |
| ES219 | 5471 | M00085223A:G01 | B-30523 |
| ES219 | 5471 | M00084740C:B08 | B-30523 |
| ES219 | 5471 | M00085056A:G12 | B-30523 |
| ES219 | 5471 | M00084671A:C12 | B-30523 |
| ES219 | 5471 | M00084571C:D05 | B-30523 |
| ES219 | 5471 | M00084587B:H07 | B-30523 |
| ES219 | 5471 | M00084582C:H03 | B-30523 |
| ES219 | 5471 | M00084618C:A03 | B-30523 |
| ES219 | 5471 | M00084687A:A03 | B-30523 |
| ES219 | 5471 | M00085038A:C06 | B-30523 |
| ES219 | 5471 | M00084722A:H12 | B-30523 |
| ES219 | 5471 | M00084676B:E02 | B-30523 |
| ES219 | 5471 | M00084615D:H12 | B-30523 |
| ES219 | 5471 | M00084659C:G05 | B-30523 |
| ES219 | 5471 | M00084536B:A03 | B-30523 |
| ES219 | 5471 | M00084929C:B02 | B-30523 |
| ES219 | 5471 | M00084652D:G11 | B-30523 |
| ES219 | 5471 | M00084611B:A11 | B-30523 |
| ES219 | 5471 | M00084530D:G07 | B-30523 |
| ES219 | 5471 | M00084527C:H07 | B-30523 |
| ES219 | 5471 | M00084545C:C05 | B-30523 |
| ES219 | 5471 | M00084535D:C12 | B-30523 |
| ES219 | 5471 | M00084684C:D02 | B-30523 |
| ES219 | 5471 | M00084679D:G12 | B-30523 |
| ES219 | 5471 | M00084734A:E04 | B-30523 |
| ES219 | 5471 | M00084696D:H04 | B-30523 |
| ES220 | 5472 | M00084724D:F04 | B-30524 |
| ES220 | 5472 | M00084559B:F10 | B-30524 |
| ES220 | 5472 | M00084707D:H03 | B-30524 |
| ES220 | 5472 | M00084525D:H01 | B-30524 |
| ES220 | 5472 | M00084578C:G09 | B-30524 |
| ES220 | 5472 | M00084710B:G07 | B-30524 |
| ES220 | 5472 | M00084537B:C05 | B-30524 |
| ES220 | 5472 | M00084560A:G08 | B-30524 |
| ES220 | 5472 | M00085129B:C02 | B-30524 |
| ES220 | 5472 | M00084620D:E05 | B-30524 |
| ES220 | 5472 | M00084576A:E12 | B-30524 |
| ES220 | 5472 | M00084720A:A01 | B-30524 |
| ES220 | 5472 | M00084654A:E04 | B-30524 |
| ES220 | 5472 | M00084596D:E10 | B-30524 |
| ES220 | 5472 | M00084646A:D02 | B-30524 |
| ES220 | 5472 | M00084572D:F07 | B-30524 |
| ES220 | 5472 | M00084620A:E08 | B-30524 |
| ES220 | 5472 | M00084553B:F04 | B-30524 |
| ES220 | 5472 | M00084614D:B07 | B-30524 |
| ES220 | 5472 | M00084604D:D08 | B-30524 |
| ES220 | 5472 | M00084722D:A03 | B-30524 |
| ES220 | 5472 | M00084958C:B03 | B-30524 |
| ES220 | 5472 | M00084523C:A05 | B-30524 |
| ES220 | 5472 | M00085166C:A08 | B-30524 |
| ES220 | 5472 | M00084467A:D06 | B-30524 |
| ES220 | 5472 | M00084890C:A06 | B-30524 |
| ES220 | 5472 | M00084609C:F10 | B-30524 |
| ES220 | 5472 | M00084413C:A11 | B-30524 |
| ES220 | 5472 | M00084834A:A03 | B-30524 |
| ES220 | 5472 | M00085172A:G05 | B-30524 |
| ES220 | 5472 | M00085146B:C01 | B-30524 |
| ES220 | 5472 | M00085038A:B10 | B-30524 |
| ES220 | 5472 | M00084246A:D03 | B-30524 |
| ES220 | 5472 | M00084967B:D09 | B-30524 |
| ES220 | 5472 | M00085035D:E04 | B-30524 |
| ES220 | 5472 | M00084736B:H03 | B-30524 |
| ES220 | 5472 | M00085025A:D11 | B-30524 |
| ES220 | 5472 | M00084900C:A04 | B-30524 |
| ES220 | 5472 | M00085127C:C03 | B-30524 |
| ES220 | 5472 | M00084424A:G07 | B-30524 |
| ES220 | 5472 | M00085131D:A06 | B-30524 |
| ES220 | 5472 | M00084987B:H12 | B-30524 |
| ES220 | 5472 | M00084967C:D10 | B-30524 |
| ES220 | 5472 | M00084420A:G02 | B-30524 |
| ES220 | 5472 | M00084452B:F07 | B-30524 |
| ES220 | 5472 | M00084705C:D01 | B-30524 |
| ES220 | 5472 | M00085156A:G04 | B-30524 |
| ES220 | 5472 | M00084447D:F03 | B-30524 |
| ES220 | 5472 | M00084495B:C11 | B-30524 |
| ES220 | 5472 | M00084745A:A08 | B-30524 |
| ES220 | 5472 | M00084458B:G05 | B-30524 |
| ES220 | 5472 | M00084449C:C01 | B-30524 |
| ES220 | 5472 | M00084867B:A03 | B-30524 |
| ES220 | 5472 | M00084680A:F08 | B-30524 |
| ES220 | 5472 | M00084585B:D06 | B-30524 |
| ES220 | 5472 | M00084835D:H03 | B-30524 |
| ES220 | 5472 | M00084685C:B12 | B-30524 |
| ES220 | 5472 | M00084500C:D01 | B-30524 |
| ES220 | 5472 | M00084469A:C09 | B-30524 |
| ES220 | 5472 | M00084381C:A05 | B-30524 |
| ES220 | 5472 | M00084477C:C07 | B-30524 |
| ES220 | 5472 | M00084647C:A05 | B-30524 |
| ES220 | 5472 | M00084687C:F12 | B-30524 |
| ES220 | 5472 | M00084756C:H01 | B-30524 |
| ES220 | 5472 | M00084565D:F08 | B-30524 |
| ES220 | 5472 | M00084560B:F12 | B-30524 |
| ES220 | 5472 | M00084640D:A08 | B-30524 |
| ES220 | 5472 | M00084443A:E10 | B-30524 |
| ES220 | 5472 | M00084521A:E11 | B-30524 |
| ES220 | 5472 | M00085019C:D05 | B-30524 |
| ES220 | 5472 | M00084587C:A07 | B-30524 |
| ES220 | 5472 | M00084616A:G03 | B-30524 |
| ES220 | 5472 | M00084732B:A04 | B-30524 |
| ES220 | 5472 | M00084666A:C04 | B-30524 |
| ES220 | 5472 | M00084633A:H05 | B-30524 |
| ES220 | 5472 | M00084510C:F05 | B-30524 |
| ES220 | 5472 | M00084648B:F06 | B-30524 |
| ES220 | 5472 | M00084700D:H04 | B-30524 |
| ES220 | 5472 | M00084506C:A05 | B-30524 |
| ES220 | 5472 | M00084475C:G11 | B-30524 |
| ES220 | 5472 | M00084673B:H11 | B-30524 |
| ES220 | 5472 | M00084595D:D08 | B-30524 |
| ES220 | 5472 | M00084636C:A06 | B-30524 |
| ES220 | 5472 | M00084612C:B01 | B-30524 |
| ES220 | 5472 | M00084644A:H05 | B-30524 |
| ES220 | 5472 | M00084602D:B09 | B-30524 |
| ES220 | 5472 | M00084584B:G07 | B-30524 |
| ES220 | 5472 | M00084678C:C11 | B-30524 |
| ES220 | 5472 | M00084546C:C06 | B-30524 |
| ES220 | 5472 | M00084755A:D02 | B-30524 |
| ES220 | 5472 | M00084536D:F07 | B-30524 |
| ES220 | 5472 | M00084699A:G05 | B-30524 |
| ES220 | 5472 | M00084438D:H04 | B-30524 |
| ES220 | 5472 | M00084766B:F02 | B-30524 |
| ES220 | 5472 | M00084703B:D09 | B-30524 |
| ES220 | 5472 | M00084856B:D03 | B-30524 |
| ES220 | 5472 | M00084857A:G05 | B-30524 |
| ES220 | 5472 | M00084868B:D01 | B-30524 |
| ES220 | 5472 | M00084823D:E05 | B-30524 |
| ES220 | 5472 | M00084485C:B04 | B-30524 |
| ES220 | 5472 | M00084910D:E07 | B-30524 |
| ES220 | 5472 | M00084996B:D08 | B-30524 |
| ES220 | 5472 | M00084487D:F07 | B-30524 |
| ES220 | 5472 | M00084824C:C10 | B-30524 |
| ES220 | 5472 | M00084949B:B12 | B-30524 |
| ES220 | 5472 | M00084746B:B04 | B-30524 |
| ES220 | 5472 | M00084944D:E05 | B-30524 |
| ES220 | 5472 | M00084851C:F10 | B-30524 |
| ES220 | 5472 | M00084849A:H08 | B-30524 |
| ES220 | 5472 | M00084843A:G01 | B-30524 |
| ES220 | 5472 | M00084921C:E04 | B-30524 |
| ES220 | 5472 | M00084742A:F07 | B-30524 |
| ES220 | 5472 | M00083799D:F10 | B-30524 |
| ES220 | 5472 | M00084760D:D09 | B-30524 |
| ES220 | 5472 | M00084845C:H05 | B-30524 |
| ES220 | 5472 | M00084927A:C01 | B-30524 |
| ES220 | 5472 | M00084935B:E10 | B-30524 |
| ES220 | 5472 | M00084974D:F11 | B-30524 |
| ES220 | 5472 | M00084935C:E07 | B-30524 |
| ES220 | 5472 | M00084503D:G10 | B-30524 |
| ES220 | 5472 | M00084907C:C01 | B-30524 |
| ES220 | 5472 | M00084893C:B01 | B-30524 |
| ES220 | 5472 | M00083803B:F11 | B-30524 |
| ES220 | 5472 | M00084945A:D10 | B-30524 |
| ES220 | 5472 | M00084765B:A10 | B-30524 |
| ES220 | 5472 | M00084455D:G03 | B-30524 |
| ES220 | 5472 | M00084874D:E03 | B-30524 |
| ES220 | 5472 | M00084889C:A04 | B-30524 |
| ES220 | 5472 | M00084846B:H07 | B-30524 |
| ES220 | 5472 | M00084967B:B10 | B-30524 |
| ES220 | 5472 | M00084838A:F12 | B-30524 |
| ES220 | 5472 | M00084885A:C01 | B-30524 |
| ES220 | 5472 | M00084823A:H06 | B-30524 |
| ES220 | 5472 | M00084958B:E10 | B-30524 |
| ES220 | 5472 | M00084399B:E05 | B-30524 |
| ES220 | 5472 | M00084880B:D03 | B-30524 |
| ES220 | 5472 | M00084877D:G07 | B-30524 |
| ES220 | 5472 | M00084406B:C03 | B-30524 |
| ES220 | 5472 | M00084856B:A12 | B-30524 |
| ES220 | 5472 | M00084888D:A11 | B-30524 |
| ES220 | 5472 | M00083831D:H11 | B-30524 |
| ES220 | 5472 | M00084481D:C06 | B-30524 |
| ES220 | 5472 | M00083834B:F09 | B-30524 |
| ES220 | 5472 | M00084707D:B08 | B-30524 |
| ES220 | 5472 | M00084976C:C12 | B-30524 |
| ES220 | 5472 | M00085201C:C12 | B-30524 |
| ES220 | 5472 | M00084379D:A05 | B-30524 |
| ES220 | 5472 | M00084392C:G06 | B-30524 |
| ES220 | 5472 | M00084492B:F03 | B-30524 |
| ES220 | 5472 | M00085697B:G05 | B-30524 |
| ES220 | 5472 | M00085683B:B10 | B-30524 |
| ES220 | 5472 | M00084988B:B08 | B-30524 |
| ES220 | 5472 | M00084969C:H11 | B-30524 |
| ES220 | 5472 | M00084988C:G03 | B-30524 |
| ES220 | 5472 | M00085123A:E07 | B-30524 |
| ES220 | 5472 | M00084988C:A04 | B-30524 |
| ES220 | 5472 | M00084363A:C02 | B-30524 |
| ES220 | 5472 | M00084975A:G05 | B-30524 |
| ES220 | 5472 | M00084431C:G08 | B-30524 |
| ES220 | 5472 | M00084972B:H03 | B-30524 |
| ES220 | 5472 | M00084376A:E06 | B-30524 |
| ES220 | 5472 | M00084859C:D09 | B-30524 |
| ES220 | 5472 | M00084957B:H07 | B-30524 |
| ES220 | 5472 | M00085053D:D04 | B-30524 |
| ES220 | 5472 | M00084425A:A01 | B-30524 |
| ES220 | 5472 | M00084367D:E06 | B-30524 |
| ES220 | 5472 | M00084938B:A11 | B-30524 |
| ES220 | 5472 | M00085051A:G03 | B-30524 |
| ES220 | 5472 | M00083817B:G09 | B-30524 |
| ES220 | 5472 | M00085229B:C10 | B-30524 |
| ES220 | 5472 | M00085178D:F01 | B-30524 |
| ES220 | 5472 | M00084980D:D02 | B-30524 |
| ES220 | 5472 | M00085228B:C10 | B-30524 |
| ES220 | 5472 | M00085243A:D07 | B-30524 |
| ES220 | 5472 | M00085031B:E03 | B-30524 |
| ES220 | 5472 | M00085164C:G05 | B-30524 |
| ES220 | 5472 | M00085031C:D05 | B-30524 |
| ES220 | 5472 | M00084251D:C05 | B-30524 |
| ES220 | 5472 | M00085027A:C02 | B-30524 |
| ES220 | 5472 | M00084248D:H09 | B-30524 |
| ES220 | 5472 | M00085209C:F11 | B-30524 |
| ES220 | 5472 | M00084368D:D03 | B-30524 |
| ES220 | 5472 | M00083818A:E09 | B-30524 |
| ES220 | 5472 | M00084980C:E06 | B-30524 |
| ES220 | 5472 | M00084248B:C06 | B-30524 |
| ES220 | 5472 | M00085244C:D03 | B-30524 |
| ES220 | 5472 | M00084987A:D09 | B-30524 |
| ES220 | 5472 | M00084994A:H04 | B-30524 |
| ES220 | 5472 | M00084970D:E08 | B-30524 |
| ES220 | 5472 | M00085038D:D10 | B-30524 |
| ES220 | 5472 | M00085035B:C12 | B-30524 |
| ES220 | 5472 | M00085184D:B08 | B-30524 |
| ES221 | 5473 | M00084666C:A06 | B-30525 |
| ES221 | 5473 | M00084657C:E01 | B-30525 |
| ES221 | 5473 | M00084540B:B08 | B-30525 |
| ES221 | 5473 | M00084415C:C05 | B-30525 |
| ES221 | 5473 | M00084812A:C02 | B-30525 |
| ES221 | 5473 | M00084396B:B03 | B-30525 |
| ES221 | 5473 | M00084844B:H08 | B-30525 |
| ES221 | 5473 | M00084877D:H09 | B-30525 |
| ES221 | 5473 | M00084925C:G01 | B-30525 |
| ES221 | 5473 | M00084970A:C11 | B-30525 |
| ES221 | 5473 | M00084961C:F01 | B-30525 |
| ES221 | 5473 | M00084391B:D06 | B-30525 |
| ES221 | 5473 | M00084694D:F04 | B-30525 |
| ES221 | 5473 | M00084698B:D02 | B-30525 |
| ES221 | 5473 | M00084388A:G03 | B-30525 |
| ES221 | 5473 | M00084973A:C01 | B-30525 |
| ES221 | 5473 | M00084423C:G11 | B-30525 |
| ES221 | 5473 | M00084497D:D03 | B-30525 |
| ES221 | 5473 | M00084889D:G06 | B-30525 |
| ES221 | 5473 | M00084959B:C07 | B-30525 |
| ES221 | 5473 | M00084432B:C05 | B-30525 |
| ES221 | 5473 | M00084489A:D12 | B-30525 |
| ES221 | 5473 | M00084748A:D09 | B-30525 |
| ES221 | 5473 | M00084962C:F10 | B-30525 |
| ES221 | 5473 | M00084767B:D10 | B-30525 |
| ES221 | 5473 | M00084711B:A05 | B-30525 |
| ES221 | 5473 | M00084743A:E03 | B-30525 |
| ES221 | 5473 | M00084466B:E01 | B-30525 |
| ES221 | 5473 | M00084450C:A09 | B-30525 |
| ES221 | 5473 | M00084492C:B05 | B-30525 |
| ES221 | 5473 | M00084487B:A06 | B-30525 |
| ES221 | 5473 | M00084480B:A05 | B-30525 |
| ES221 | 5473 | M00084764D:G08 | B-30525 |
| ES221 | 5473 | M00084743D:H04 | B-30525 |
| ES221 | 5473 | M00084891D:A02 | B-30525 |
| ES221 | 5473 | M00084822C:D06 | B-30525 |
| ES221 | 5473 | M00084853D:A12 | B-30525 |
| ES221 | 5473 | M00084822B:G11 | B-30525 |
| ES221 | 5473 | M00084756D:C04 | B-30525 |
| ES221 | 5473 | M00084839C:B09 | B-30525 |
| ES221 | 5473 | M00084767D:B04 | B-30525 |
| ES221 | 5473 | M00084703A:E04 | B-30525 |
| ES221 | 5473 | M00084853A:F08 | B-30525 |
| ES221 | 5473 | M00084956C:G09 | B-30525 |
| ES221 | 5473 | M00084908C:F07 | B-30525 |
| ES221 | 5473 | M00084902B:A10 | B-30525 |
| ES221 | 5473 | M00084833D:B04 | B-30525 |
| ES221 | 5473 | M00085023D:E11 | B-30525 |
| ES221 | 5473 | M00085151A:B04 | B-30525 |
| ES221 | 5473 | M00085039D:F09 | B-30525 |
| ES221 | 5473 | M00085169A:H12 | B-30525 |
| ES221 | 5473 | M00085052B:E04 | B-30525 |
| ES221 | 5473 | M00085171D:F05 | B-30525 |
| ES221 | 5473 | M00085050A:B06 | B-30525 |
| ES221 | 5473 | M00085155B:F10 | B-30525 |
| ES221 | 5473 | M00085123C:G11 | B-30525 |
| ES221 | 5473 | M00085182B:H10 | B-30525 |
| ES221 | 5473 | M00084675A:E02 | B-30525 |
| ES221 | 5473 | M00085248B:G12 | B-30525 |
| ES221 | 5473 | M00084731C:G07 | B-30525 |
| ES221 | 5473 | M00085701A:A09 | B-30525 |
| ES221 | 5473 | M00085246B:G12 | B-30525 |
| ES221 | 5473 | M00084967C:D12 | B-30525 |
| ES221 | 5473 | M00085190B:C09 | B-30525 |
| ES221 | 5473 | M00085167C:D06 | B-30525 |
| ES221 | 5473 | M00085705A:E01 | B-30525 |
| ES221 | 5473 | M00085214D:G01 | B-30525 |
| ES221 | 5473 | M00084755D:E06 | B-30525 |
| ES221 | 5473 | M00084630D:F09 | B-30525 |
| ES221 | 5473 | M00085191A:B03 | B-30525 |
| ES221 | 5473 | M00085143C:D05 | B-30525 |
| ES221 | 5473 | M00084886A:C06 | B-30525 |
| ES221 | 5473 | M00083803B:F12 | B-30525 |
| ES221 | 5473 | M00084949B:H11 | B-30525 |
| ES221 | 5473 | M00084701C:E08 | B-30525 |
| ES221 | 5473 | M00084945A:H10 | B-30525 |
| ES221 | 5473 | M00084667C:A03 | B-30525 |
| ES221 | 5473 | M00084953D:D03 | B-30525 |
| ES221 | 5473 | M00084539D:D11 | B-30525 |
| ES221 | 5473 | M00084737A:C09 | B-30525 |
| ES221 | 5473 | M00084968C:D10 | B-30525 |
| ES221 | 5473 | M00084670B:A09 | B-30525 |
| ES221 | 5473 | M00085167A:G02 | B-30525 |
| ES221 | 5473 | M00084554C:D05 | B-30525 |
| ES221 | 5473 | M00085145C:D02 | B-30525 |
| ES221 | 5473 | M00084722D:G04 | B-30525 |
| ES221 | 5473 | M00084721C:F09 | B-30525 |
| ES221 | 5473 | M00084866B:A03 | B-30525 |
| ES221 | 5473 | M00084727A:A02 | B-30525 |
| ES221 | 5473 | M00084407A:H09 | B-30525 |
| ES221 | 5473 | M00084855D:H05 | B-30525 |
| ES221 | 5473 | M00084403D:D04 | B-30525 |
| ES221 | 5473 | M00085144D:G03 | B-30525 |
| ES221 | 5473 | M00084880D:A10 | B-30525 |
| ES221 | 5473 | M00084958C:B03 | B-30525 |
| ES221 | 5473 | M00084888C:D12 | B-30525 |
| ES221 | 5473 | M00084587C:G07 | B-30525 |
| ES221 | 5473 | M00083844C:C04 | B-30525 |
| ES221 | 5473 | M00084647D:C05 | B-30525 |
| ES221 | 5473 | M00084528C:F06 | B-30525 |
| ES221 | 5473 | M00084857D:A11 | B-30525 |
| ES221 | 5473 | M00084385A:D02 | B-30525 |
| ES221 | 5473 | M00084561C:D07 | B-30525 |
| ES221 | 5473 | M00084994D:H04 | B-30525 |
| ES221 | 5473 | M00084448B:D11 | B-30525 |
| ES221 | 5473 | M00085006D:C10 | B-30525 |
| ES221 | 5473 | M00084580B:B05 | B-30525 |
| ES221 | 5473 | M00083814D:A10 | B-30525 |
| ES221 | 5473 | M00084970C:G03 | B-30525 |
| ES221 | 5473 | M00084372D:H11 | B-30525 |
| ES221 | 5473 | M00084377B:E11 | B-30525 |
| ES221 | 5473 | M00085230B:G08 | B-30525 |
| ES221 | 5473 | M00084584B:F09 | B-30525 |
| ES221 | 5473 | M00084584B:H12 | B-30525 |
| ES221 | 5473 | M00085249C:C11 | B-30525 |
| ES221 | 5473 | M00084441B:E05 | B-30525 |
| ES221 | 5473 | M00083841A:G01 | B-30525 |
| ES221 | 5473 | M00085006D:C04 | B-30525 |
| ES221 | 5473 | M00084686B:B04 | B-30525 |
| ES221 | 5473 | M00084998A:C12 | B-30525 |
| ES221 | 5473 | M00085034B:E11 | B-30525 |
| ES221 | 5473 | M00084683B:A01 | B-30525 |
| ES221 | 5473 | M00084613A:A01 | B-30525 |
| ES221 | 5473 | M00084633B:A06 | B-30525 |
| ES221 | 5473 | M00085032C:F04 | B-30525 |
| ES221 | 5473 | M00085022B:F05 | B-30525 |
| ES221 | 5473 | M00084509A:E10 | B-30525 |
| ES221 | 5473 | M00084400A:B09 | B-30525 |
| ES221 | 5473 | M00084677C:F03 | B-30525 |
| ES221 | 5473 | M00084427B:D01 | B-30525 |
| ES221 | 5473 | M00083844B:C04 | B-30525 |
| ES221 | 5473 | M00084598D:H05 | B-30525 |
| ES221 | 5473 | M00084443B:C02 | B-30525 |
| ES221 | 5473 | M00084514A:A03 | B-30525 |
| ES221 | 5473 | M00084560C:G05 | B-30525 |
| ES221 | 5473 | M00084504C:F05 | B-30525 |
| ES221 | 5473 | M00084517C:D06 | B-30525 |
| ES221 | 5473 | M00084420C:D03 | B-30525 |
| ES221 | 5473 | M00084524D:D02 | B-30525 |
| ES221 | 5473 | M00084499D:A10 | B-30525 |
| ES221 | 5473 | M00085022B:B03 | B-30525 |
| ES221 | 5473 | M00084958B:E10 | B-30525 |
| ES221 | 5473 | M00084513C:C10 | B-30525 |
| ES221 | 5473 | M00084595B:C08 | B-30525 |
| ES221 | 5473 | M00083804A:H12 | B-30525 |
| ES221 | 5473 | M00084859D:B03 | B-30525 |
| ES221 | 5473 | M00084641B:F08 | B-30525 |
| ES221 | 5473 | M00084844A:E10 | B-30525 |
| ES221 | 5473 | M00083838A:E05 | B-30525 |
| ES221 | 5473 | M00083849C:F11 | B-30525 |
| ES221 | 5473 | M00084461C:D06 | B-30525 |
| ES221 | 5473 | M00084810D:B10 | B-30525 |
| ES221 | 5473 | M00085047D:F08 | B-30525 |
| ES221 | 5473 | M00084912D:G06 | B-30525 |
| ES221 | 5473 | M00084645C:F07 | B-30525 |
| ES221 | 5473 | M00084912D:A09 | B-30525 |
| ES221 | 5473 | M00084862B:B01 | B-30525 |
| ES221 | 5473 | M00084938C:G06 | B-30525 |
| ES221 | 5473 | M00084534B:E12 | B-30525 |
| ES221 | 5473 | M00084909C:G02 | B-30525 |
| ES221 | 5473 | M00084973A:A01 | B-30525 |
| ES221 | 5473 | M00084651B:G10 | B-30525 |
| ES221 | 5473 | M00084925A:B08 | B-30525 |
| ES221 | 5473 | M00084568D:A02 | B-30525 |
| ES221 | 5473 | M00084456A:H04 | B-30525 |
| ES221 | 5473 | M00084988C:B01 | B-30525 |
| ES221 | 5473 | M00084842C:B07 | B-30525 |
| ES221 | 5473 | M00084708A:A11 | B-30525 |
| ES221 | 5473 | M00084602C:E04 | B-30525 |
| ES221 | 5473 | M00084757B:F11 | B-30525 |
| ES221 | 5473 | M00084483A:C06 | B-30525 |
| ES221 | 5473 | M00084605B:H04 | B-30525 |
| ES221 | 5473 | M00083812C:G02 | B-30525 |
| ES221 | 5473 | M00084610D:H04 | B-30525 |
| ES221 | 5473 | M00085056D:B12 | B-30525 |
| ES221 | 5473 | M00085017C:A11 | B-30525 |
| ES221 | 5473 | M00084573A:A10 | B-30525 |
| ES221 | 5473 | M00084637B:E01 | B-30525 |
| ES221 | 5473 | M00085056B:B06 | B-30525 |
| ES221 | 5473 | M00084510C:H01 | B-30525 |
| ES221 | 5473 | M00084577B:C08 | B-30525 |
| ES221 | 5473 | M00084646B:B03 | B-30525 |
| ES221 | 5473 | M00084844C:F04 | B-30525 |
| ES221 | 5473 | M00084894B:F11 | B-30525 |
| ES221 | 5473 | M00084930B:E12 | B-30525 |
| ES221 | 5473 | M00084469B:F08 | B-30525 |
| ES221 | 5473 | M00084569D:B04 | B-30525 |
| ES221 | 5473 | M00084453D:B12 | B-30525 |
| ES221 | 5473 | M00083844A:E12 | B-30525 |
| ES221 | 5473 | M00085009D:A02 | B-30525 |
| ES221 | 5473 | M00084619A:E04 | B-30525 |
| ES221 | 5473 | M00085006C:C07 | B-30525 |
| ES222 | 5474 | M00084459A:F10 | B-30526 |
| ES222 | 5474 | M00084721B:C11 | B-30526 |
| ES222 | 5474 | M00084454A:G08 | B-30526 |
| ES222 | 5474 | M00084460D:B04 | B-30526 |
| ES222 | 5474 | M00084723D:G09 | B-30526 |
| ES222 | 5474 | M00084704A:C12 | B-30526 |
| ES222 | 5474 | M00084487C:H06 | B-30526 |
| ES222 | 5474 | M00084867C:G02 | B-30526 |
| ES222 | 5474 | M00084475B:D03 | B-30526 |
| ES222 | 5474 | M00084490A:C12 | B-30526 |
| ES222 | 5474 | M00084865D:G02 | B-30526 |
| ES222 | 5474 | M00084876D:A06 | B-30526 |
| ES222 | 5474 | M00084553D:G05 | B-30526 |
| ES222 | 5474 | M00084558D:A04 | B-30526 |
| ES222 | 5474 | M00084645B:A06 | B-30526 |
| ES222 | 5474 | M00084747D:G02 | B-30526 |
| ES222 | 5474 | M00084884D:D03 | B-30526 |
| ES222 | 5474 | M00084700A:C10 | B-30526 |
| ES222 | 5474 | M00084973A:C01 | B-30526 |
| ES222 | 5474 | M00084493A:E03 | B-30526 |
| ES222 | 5474 | M00084497B:C12 | B-30526 |
| ES222 | 5474 | M00084500D:B11 | B-30526 |
| ES222 | 5474 | M00084523C:C10 | B-30526 |
| ES222 | 5474 | M00084526D:E09 | B-30526 |
| ES222 | 5474 | M00084923D:B05 | B-30526 |
| ES222 | 5474 | M00084962C:F10 | B-30526 |
| ES222 | 5474 | M00084669C:A10 | B-30526 |
| ES222 | 5474 | M00084444D:F09 | B-30526 |
| ES222 | 5474 | M00084757B:F05 | B-30526 |
| ES222 | 5474 | M00084922A:C08 | B-30526 |
| ES222 | 5474 | M00084960D:D02 | B-30526 |
| ES222 | 5474 | M00084837B:E06 | B-30526 |
| ES222 | 5474 | M00084763D:A04 | B-30526 |
| ES222 | 5474 | M00084651C:H01 | B-30526 |
| ES222 | 5474 | M00084441D:E09 | B-30526 |
| ES222 | 5474 | M00084509D:C02 | B-30526 |
| ES222 | 5474 | M00084510D:D05 | B-30526 |
| ES222 | 5474 | M00084657D:B10 | B-30526 |
| ES222 | 5474 | M00084946D:H05 | B-30526 |
| ES222 | 5474 | M00084506A:E08 | B-30526 |
| ES222 | 5474 | M00084420D:C07 | B-30526 |
| ES222 | 5474 | M00085247A:F05 | B-30526 |
| ES222 | 5474 | M00085142D:F04 | B-30526 |
| ES222 | 5474 | M00085151A:H09 | B-30526 |
| ES222 | 5474 | M00085029D:E12 | B-30526 |
| ES222 | 5474 | M00085141C:G06 | B-30526 |
| ES222 | 5474 | M00085035D:D09 | B-30526 |
| ES222 | 5474 | M00085168D:D04 | B-30526 |
| ES222 | 5474 | M00084995B:B08 | B-30526 |
| ES222 | 5474 | M00085008B:H11 | B-30526 |
| ES222 | 5474 | M00085059B:H11 | B-30526 |
| ES222 | 5474 | M00085125C:H06 | B-30526 |
| ES222 | 5474 | M00084890B:E02 | B-30526 |
| ES222 | 5474 | M00084418D:A04 | B-30526 |
| ES222 | 5474 | M00084961C:A06 | B-30526 |
| ES222 | 5474 | M00084766B:E03 | B-30526 |
| ES222 | 5474 | M00084406A:B03 | B-30526 |
| ES222 | 5474 | M00085686A:C05 | B-30526 |
| ES222 | 5474 | M00085124A:G04 | B-30526 |
| ES222 | 5474 | M00085059B:H07 | B-30526 |
| ES222 | 5474 | M00084646B:D07 | B-30526 |
| ES222 | 5474 | M00084246B:D10 | B-30526 |
| ES222 | 5474 | M00084974D:F11 | B-30526 |
| ES222 | 5474 | M00084738B:A09 | B-30526 |
| ES222 | 5474 | M00085015A:C09 | B-30526 |
| ES222 | 5474 | M00083839B:G09 | B-30526 |
| ES222 | 5474 | M00085012C:D06 | B-30526 |
| ES222 | 5474 | M00085058A:H02 | B-30526 |
| ES222 | 5474 | M00085009C:C01 | B-30526 |
| ES222 | 5474 | M00083818C:A02 | B-30526 |
| ES222 | 5474 | M00085007B:C07 | B-30526 |
| ES222 | 5474 | M00085047A:H02 | B-30526 |
| ES222 | 5474 | M00085245C:D07 | B-30526 |
| ES222 | 5474 | M00085032D:C03 | B-30526 |
| ES222 | 5474 | M00085039B:E09 | B-30526 |
| ES222 | 5474 | M00085053C:D07 | B-30526 |
| ES222 | 5474 | M00084364D:F08 | B-30526 |
| ES222 | 5474 | M00085152B:A06 | B-30526 |
| ES222 | 5474 | M00084969C:F03 | B-30526 |
| ES222 | 5474 | M00084896B:G10 | B-30526 |
| ES222 | 5474 | M00084427C:D04 | B-30526 |
| ES222 | 5474 | M00085018C:B09 | B-30526 |
| ES222 | 5474 | M00084668D:D08 | B-30526 |
| ES222 | 5474 | M00085175B:A03 | B-30526 |
| ES222 | 5474 | M00084437C:G05 | B-30526 |
| ES222 | 5474 | M00084967B:B10 | B-30526 |
| ES222 | 5474 | M00084998B:A04 | B-30526 |
| ES222 | 5474 | M00084716D:H03 | B-30526 |
| ES222 | 5474 | M00084708B:A06 | B-30526 |
| ES222 | 5474 | M00084755B:A04 | B-30526 |
| ES222 | 5474 | M00084380C:C09 | B-30526 |
| ES222 | 5474 | M00085013A:E06 | B-30526 |
| ES222 | 5474 | M00084374A:A10 | B-30526 |
| ES222 | 5474 | M00085194C:B12 | B-30526 |
| ES222 | 5474 | M00084971C:G07 | B-30526 |
| ES222 | 5474 | M00084515D:G03 | B-30526 |
| ES222 | 5474 | M00084961D:H03 | B-30526 |
| ES222 | 5474 | M00084908D:B11 | B-30526 |
| ES222 | 5474 | M00084702A:B08 | B-30526 |
| ES222 | 5474 | M00083838C:F07 | B-30526 |
| ES222 | 5474 | M00084390B:H04 | B-30526 |
| ES222 | 5474 | M00084734A:H01 | B-30526 |
| ES222 | 5474 | M00083817B:A11 | B-30526 |
| ES222 | 5474 | M00085176C:B11 | B-30526 |
| ES222 | 5474 | M00084902C:F05 | B-30526 |
| ES222 | 5474 | M00085677A:E02 | B-30526 |
| ES222 | 5474 | M00084948D:B08 | B-30526 |
| ES222 | 5474 | M00085190B:H04 | B-30526 |
| ES222 | 5474 | M00084820D:A03 | B-30526 |
| ES222 | 5474 | M00084479B:E04 | B-30526 |
| ES222 | 5474 | M00084408D:E06 | B-30526 |
| ES222 | 5474 | M00085009B:F10 | B-30526 |
| ES222 | 5474 | M00085697A:F01 | B-30526 |
| ES222 | 5474 | M00084423D:B05 | B-30526 |
| ES222 | 5474 | M00084973A:A01 | B-30526 |
| ES222 | 5474 | M00083804B:C03 | B-30526 |
| ES222 | 5474 | M00084841B:H09 | B-30526 |
| ES222 | 5474 | M00084685D:B11 | B-30526 |
| ES222 | 5474 | M00084599D:C02 | B-30526 |
| ES222 | 5474 | M00084573D:G11 | B-30526 |
| ES222 | 5474 | M00084603A:B07 | B-30526 |
| ES222 | 5474 | M00084823D:E06 | B-30526 |
| ES222 | 5474 | M00084565A:D10 | B-30526 |
| ES222 | 5474 | M00084767B:F06 | B-30526 |
| ES222 | 5474 | M00084963D:D07 | B-30526 |
| ES222 | 5474 | M00084611A:A06 | B-30526 |
| ES222 | 5474 | M00084829B:F06 | B-30526 |
| ES222 | 5474 | M00084850C:A11 | B-30526 |
| ES222 | 5474 | M00084540D:B12 | B-30526 |
| ES222 | 5474 | M00084614C:G05 | B-30526 |
| ES222 | 5474 | M00084826B:D12 | B-30526 |
| ES222 | 5474 | M00084605D:G09 | B-30526 |
| ES222 | 5474 | M00084923D:F11 | B-30526 |
| ES222 | 5474 | M00084664D:E05 | B-30526 |
| ES222 | 5474 | M00084533A:C04 | B-30526 |
| ES222 | 5474 | M00084843D:F05 | B-30526 |
| ES222 | 5474 | M00084894A:G09 | B-30526 |
| ES222 | 5474 | M00084913B:F05 | B-30526 |
| ES222 | 5474 | M00083817D:A08 | B-30526 |
| ES222 | 5474 | M00084451D:A03 | B-30526 |
| ES222 | 5474 | M00084675B:A04 | B-30526 |
| ES222 | 5474 | M00084889A:B07 | B-30526 |
| ES222 | 5474 | M00084879A:A04 | B-30526 |
| ES222 | 5474 | M00084638D:A05 | B-30526 |
| ES222 | 5474 | M00084468A:A09 | B-30526 |
| ES222 | 5474 | M00084634A:D01 | B-30526 |
| ES222 | 5474 | M00084577B:D04 | B-30526 |
| ES222 | 5474 | M00084860B:A01 | B-30526 |
| ES222 | 5474 | M00084567B:F03 | B-30526 |
| ES222 | 5474 | M00084619A:G10 | B-30526 |
| ES222 | 5474 | M00084683A:B12 | B-30526 |
| ES222 | 5474 | M00084631D:G01 | B-30526 |
| ES222 | 5474 | M00084520B:A12 | B-30526 |
| ES222 | 5474 | M00084886B:D06 | B-30526 |
| ES222 | 5474 | M00084727A:G09 | B-30526 |
| ES222 | 5474 | M00084393A:G07 | B-30526 |
| ES222 | 5474 | M00084571A:C02 | B-30526 |
| ES222 | 5474 | M00084866C:H04 | B-30526 |
| ES222 | 5474 | M00084449A:D09 | B-30526 |
| ES222 | 5474 | M00084857C:E11 | B-30526 |
| ES222 | 5474 | M00085226C:F08 | B-30526 |
| ES222 | 5474 | M00084392C:D03 | B-30526 |
| ES222 | 5474 | M00084389A:F12 | B-30526 |
| ES222 | 5474 | M00084696C:A07 | B-30526 |
| ES222 | 5474 | M00084397D:A09 | B-30526 |
| ES222 | 5474 | M00085173A:B07 | B-30526 |
| ES222 | 5474 | M00084252B:H01 | B-30526 |
| ES222 | 5474 | M00084970C:H09 | B-30526 |
| ES222 | 5474 | M00084648A:F08 | B-30526 |
| ES222 | 5474 | M00085245D:G07 | B-30526 |
| ES222 | 5474 | M00084642C:F10 | B-30526 |
| ES222 | 5474 | M00085220D:E06 | B-30526 |
| ES222 | 5474 | M00084745A:H04 | B-30526 |
| ES222 | 5474 | M00083809B:E08 | B-30526 |
| ES222 | 5474 | M00084940B:F06 | B-30526 |
| ES222 | 5474 | M00084533B:B10 | B-30526 |
| ES222 | 5474 | M00084970D:B01 | B-30526 |
| ES222 | 5474 | M00084583D:H12 | B-30526 |
| ES222 | 5474 | M00084585D:H12 | B-30526 |
| ES222 | 5474 | M00084581B:E06 | B-30526 |
| ES222 | 5474 | M00084588B:D02 | B-30526 |
| ES222 | 5474 | M00084919D:B08 | B-30526 |
| ES222 | 5474 | M00084812A:E05 | B-30526 |
| ES222 | 5474 | M00084768B:E09 | B-30526 |
| ES222 | 5474 | M00084748A:H02 | B-30526 |
| ES222 | 5474 | M00084519B:D01 | B-30526 |
| ES222 | 5474 | M00084926B:C05 | B-30526 |
| ES222 | 5474 | M00084847B:G05 | B-30526 |
| ES222 | 5474 | M00084858C:B01 | B-30526 |
| ES222 | 5474 | M00084483A:E05 | B-30526 |
| ES222 | 5474 | M00084596A:G03 | B-30526 |
| ES222 | 5474 | M00084681B:G11 | B-30526 |
| ES223 | 5475 | M00085368B:A02 | B-30527 |
| ES223 | 5475 | M00085365C:C09 | B-30527 |
| ES223 | 5475 | M00085317B:G09 | B-30527 |
| ES223 | 5475 | M00085732A:B09 | B-30527 |
| ES223 | 5475 | M00085649C:A12 | B-30527 |
| ES223 | 5475 | M00085337C:E09 | B-30527 |
| ES223 | 5475 | M00085520C:D02 | B-30527 |
| ES223 | 5475 | M00085358B:A04 | B-30527 |
| ES223 | 5475 | M00085640A:H11 | B-30527 |
| ES223 | 5475 | M00085344D:B07 | B-30527 |
| ES223 | 5475 | M00085314C:F01 | B-30527 |
| ES223 | 5475 | M00085334A:D10 | B-30527 |
| ES223 | 5475 | M00085262C:E04 | B-30527 |
| ES223 | 5475 | M00083750D:G12 | B-30527 |
| ES223 | 5475 | M00085255B:F11 | B-30527 |
| ES223 | 5475 | M00085701D:A02 | B-30527 |
| ES223 | 5475 | M00086280B:G09 | B-30527 |
| ES223 | 5475 | M00085628C:D08 | B-30527 |
| ES223 | 5475 | M00083726B:E07 | B-30527 |
| ES223 | 5475 | M00086285B:C10 | B-30527 |
| ES223 | 5475 | M00086084D:E12 | B-30527 |
| ES223 | 5475 | M00085446A:D04 | B-30527 |
| ES223 | 5475 | M00085697D:H11 | B-30527 |
| ES223 | 5475 | M00086085A:H03 | B-30527 |
| ES223 | 5475 | M00086057A:F07 | B-30527 |
| ES223 | 5475 | M00086196A:F07 | B-30527 |
| ES223 | 5475 | M00086279A:B07 | B-30527 |
| ES223 | 5475 | M00086266B:E10 | B-30527 |
| ES223 | 5475 | M00086280A:E02 | B-30527 |
| ES223 | 5475 | M00085309A:D11 | B-30527 |
| ES223 | 5475 | M00086291D:B08 | B-30527 |
| ES223 | 5475 | M00085861C:A03 | B-30527 |
| ES223 | 5475 | M00086247A:G11 | B-30527 |
| ES223 | 5475 | M00085373C:B06 | B-30527 |
| ES223 | 5475 | M00085332D:B03 | B-30527 |
| ES223 | 5475 | M00085632C:A06 | B-30527 |
| ES223 | 5475 | M00085360B:G03 | B-30527 |
| ES223 | 5475 | M00086191A:G09 | B-30527 |
| ES223 | 5475 | M00085473C:B02 | B-30527 |
| ES223 | 5475 | M00085600B:B03 | B-30527 |
| ES223 | 5475 | M00083749D:B09 | B-30527 |
| ES223 | 5475 | M00085301B:C10 | B-30527 |
| ES223 | 5475 | M00085278D:F03 | B-30527 |
| ES223 | 5475 | M00085296C:C09 | B-30527 |
| ES223 | 5475 | M00085435B:C05 | B-30527 |
| ES223 | 5475 | M00085533C:D11 | B-30527 |
| ES223 | 5475 | M00086061B:E02 | B-30527 |
| ES223 | 5475 | M00085284D:A09 | B-30527 |
| ES223 | 5475 | M00085566A:D12 | B-30527 |
| ES223 | 5475 | M00085528A:E02 | B-30527 |
| ES223 | 5475 | M00085336D:B10 | B-30527 |
| ES223 | 5475 | M00085315C:B03 | B-30527 |
| ES223 | 5475 | M00085509A:A02 | B-30527 |
| ES223 | 5475 | M00083698D:E01 | B-30527 |
| ES223 | 5475 | M00083701D:G09 | B-30527 |
| ES223 | 5475 | M00086155A:G12 | B-30527 |
| ES223 | 5475 | M00085293B:B05 | B-30527 |
| ES223 | 5475 | M00085728B:C08 | B-30527 |
| ES223 | 5475 | M00085611B:D03 | B-30527 |
| ES223 | 5475 | M00085592A:G06 | B-30527 |
| ES223 | 5475 | M00085304A:B11 | B-30527 |
| ES223 | 5475 | M00085266D:C09 | B-30527 |
| ES223 | 5475 | M00085335B:D09 | B-30527 |
| ES223 | 5475 | M00085707C:A10 | B-30527 |
| ES223 | 5475 | M00085555D:F08 | B-30527 |
| ES223 | 5475 | M00085588B:G10 | B-30527 |
| ES223 | 5475 | M00085264C:F04 | B-30527 |
| ES223 | 5475 | M00085733D:E05 | B-30527 |
| ES223 | 5475 | M00085647A:C08 | B-30527 |
| ES223 | 5475 | M00083714C:F04 | B-30527 |
| ES223 | 5475 | M00085707A:F01 | B-30527 |
| ES223 | 5475 | M00085548C:D04 | B-30527 |
| ES223 | 5475 | M00083745A:A10 | B-30527 |
| ES223 | 5475 | M00085396B:G04 | B-30527 |
| ES223 | 5475 | M00085449C:D04 | B-30527 |
| ES223 | 5475 | M00083698B:H01 | B-30527 |
| ES223 | 5475 | M00084772C:G12 | B-30527 |
| ES223 | 5475 | M00086126C:D09 | B-30527 |
| ES223 | 5475 | M00085808D:E01 | B-30527 |
| ES223 | 5475 | M00085927A:F06 | B-30527 |
| ES223 | 5475 | M00085814C:C12 | B-30527 |
| ES223 | 5475 | M00086015A:B03 | B-30527 |
| ES223 | 5475 | M00086146D:A09 | B-30527 |
| ES223 | 5475 | M00085076C:A07 | B-30527 |
| ES223 | 5475 | M00085427C:A04 | B-30527 |
| ES223 | 5475 | M00084774B:C10 | B-30527 |
| ES223 | 5475 | M00085432A:H08 | B-30527 |
| ES223 | 5475 | M00084796D:B01 | B-30527 |
| ES223 | 5475 | M00086127C:C05 | B-30527 |
| ES223 | 5475 | M00086160D:F08 | B-30527 |
| ES223 | 5475 | M00084802A:H09 | B-30527 |
| ES223 | 5475 | M00086081D:H11 | B-30527 |
| ES223 | 5475 | M00086106D:H01 | B-30527 |
| ES223 | 5475 | M00086159A:F05 | B-30527 |
| ES223 | 5475 | M00084782D:H08 | B-30527 |
| ES223 | 5475 | M00085956D:G04 | B-30527 |
| ES223 | 5475 | M00085770C:A12 | B-30527 |
| ES223 | 5475 | M00086008D:F08 | B-30527 |
| ES223 | 5475 | M00086018A:A05 | B-30527 |
| ES223 | 5475 | M00085761A:B03 | B-30527 |
| ES223 | 5475 | M00085751A:A11 | B-30527 |
| ES223 | 5475 | M00085956B:E08 | B-30527 |
| ES223 | 5475 | M00085955C:C03 | B-30527 |
| ES223 | 5475 | M00085904D:D02 | B-30527 |
| ES223 | 5475 | M00085899C:G10 | B-30527 |
| ES223 | 5475 | M00085927C:G10 | B-30527 |
| ES223 | 5475 | M00085896D:A11 | B-30527 |
| ES223 | 5475 | M00085892A:F04 | B-30527 |
| ES223 | 5475 | M00085882B:F11 | B-30527 |
| ES223 | 5475 | M00085419A:G09 | B-30527 |
| ES223 | 5475 | M00085962B:A12 | B-30527 |
| ES223 | 5475 | M00085811B:D12 | B-30527 |
| ES223 | 5475 | M00085986B:H02 | B-30527 |
| ES223 | 5475 | M00085922A:A08 | B-30527 |
| ES223 | 5475 | M00085854C:F04 | B-30527 |
| ES223 | 5475 | M00085835D:F06 | B-30527 |
| ES223 | 5475 | M00086183A:H04 | B-30527 |
| ES223 | 5475 | M00086193A:F04 | B-30527 |
| ES223 | 5475 | M00086197B:A03 | B-30527 |
| ES223 | 5475 | M00086203C:H04 | B-30527 |
| ES223 | 5475 | M00085827D:D01 | B-30527 |
| ES223 | 5475 | M00086097D:D12 | B-30527 |
| ES223 | 5475 | M00086294C:G05 | B-30527 |
| ES223 | 5475 | M00086176C:A06 | B-30527 |
| ES223 | 5475 | M00085825C:D12 | B-30527 |
| ES223 | 5475 | M00085849D:G06 | B-30527 |
| ES223 | 5475 | M00085817C:C10 | B-30527 |
| ES223 | 5475 | M00085807D:G11 | B-30527 |
| ES223 | 5475 | M00085839B:B12 | B-30527 |
| ES223 | 5475 | M00085750A:G03 | B-30527 |
| ES223 | 5475 | M00086248A:H09 | B-30527 |
| ES223 | 5475 | M00085964A:B11 | B-30527 |
| ES223 | 5475 | M00086003D:G08 | B-30527 |
| ES223 | 5475 | M00085819D:C02 | B-30527 |
| ES223 | 5475 | M00085066B:D12 | B-30527 |
| ES223 | 5475 | M00084775C:E05 | B-30527 |
| ES223 | 5475 | M00085083A:E04 | B-30527 |
| ES223 | 5475 | M00085090C:C09 | B-30527 |
| ES223 | 5475 | M00085100A:H07 | B-30527 |
| ES223 | 5475 | M00085101C:H03 | B-30527 |
| ES223 | 5475 | M00085105D:H02 | B-30527 |
| ES223 | 5475 | M00086323B:C04 | B-30527 |
| ES223 | 5475 | M00084808D:A07 | B-30527 |
| ES223 | 5475 | M00086003D:B10 | B-30527 |
| ES223 | 5475 | M00084787B:D12 | B-30527 |
| ES223 | 5475 | M00085068C:B03 | B-30527 |
| ES223 | 5475 | M00086291A:E10 | B-30527 |
| ES223 | 5475 | M00084807D:F07 | B-30527 |
| ES223 | 5475 | M00084799C:E08 | B-30527 |
| ES223 | 5475 | M00084781A:E05 | B-30527 |
| ES223 | 5475 | M00086324D:D01 | B-30527 |
| ES224 | 5476 | M00085641C:G01 | B-30528 |
| ES224 | 5476 | M00085623B:G11 | B-30528 |
| ES224 | 5476 | M00085805A:G07 | B-30528 |
| ES224 | 5476 | M00085846A:H10 | B-30528 |
| ES224 | 5476 | M00085369D:G04 | B-30528 |
| ES224 | 5476 | M00085817C:G05 | B-30528 |
| ES224 | 5476 | M00085635C:H07 | B-30528 |
| ES224 | 5476 | M00085907B:B11 | B-30528 |
| ES224 | 5476 | M00085650D:E12 | B-30528 |
| ES224 | 5476 | M00085854A:D09 | B-30528 |
| ES224 | 5476 | M00085628B:D03 | B-30528 |
| ES224 | 5476 | M00085393A:D06 | B-30528 |
| ES224 | 5476 | M00085620C:F05 | B-30528 |
| ES224 | 5476 | M00085750B:C10 | B-30528 |
| ES224 | 5476 | M00085773A:F05 | B-30528 |
| ES224 | 5476 | M00085933B:B06 | B-30528 |
| ES224 | 5476 | M00084804B:E01 | B-30528 |
| ES224 | 5476 | M00085337B:F08 | B-30528 |
| ES224 | 5476 | M00085349A:C08 | B-30528 |
| ES224 | 5476 | M00084783C:A09 | B-30528 |
| ES224 | 5476 | M00084782D:D10 | B-30528 |
| ES224 | 5476 | M00085273A:A09 | B-30528 |
| ES224 | 5476 | M00084794B:C01 | B-30528 |
| ES224 | 5476 | M00084780D:D07 | B-30528 |
| ES224 | 5476 | M00085345B:C09 | B-30528 |
| ES224 | 5476 | M00085344D:F01 | B-30528 |
| ES224 | 5476 | M00085747A:B11 | B-30528 |
| ES224 | 5476 | M00085814A:C02 | B-30528 |
| ES224 | 5476 | M00085503D:D05 | B-30528 |
| ES224 | 5476 | M00085304B:D11 | B-30528 |
| ES224 | 5476 | M00085900B:E02 | B-30528 |
| ES224 | 5476 | M00085859B:A11 | B-30528 |
| ES224 | 5476 | M00085860D:H02 | B-30528 |
| ES224 | 5476 | M00085649B:A03 | B-30528 |
| ES224 | 5476 | M00085815C:E11 | B-30528 |
| ES224 | 5476 | M00085941B:A06 | B-30528 |
| ES224 | 5476 | M00084800B:H09 | B-30528 |
| ES224 | 5476 | M00085919B:F02 | B-30528 |
| ES224 | 5476 | M00085449A:E02 | B-30528 |
| ES224 | 5476 | M00085344A:G08 | B-30528 |
| ES224 | 5476 | M00085520D:B11 | B-30528 |
| ES224 | 5476 | M00086035A:C11 | B-30528 |
| ES224 | 5476 | M00085955D:H10 | B-30528 |
| ES224 | 5476 | M00086175C:D06 | B-30528 |
| ES224 | 5476 | M00085510B:G12 | B-30528 |
| ES224 | 5476 | M00085927C:G11 | B-30528 |
| ES224 | 5476 | M00085919A:A05 | B-30528 |
| ES224 | 5476 | M00085985C:D02 | B-30528 |
| ES224 | 5476 | M00085934B:E12 | B-30528 |
| ES224 | 5476 | M00085389C:H04 | B-30528 |
| ES224 | 5476 | M00085406A:F03 | B-30528 |
| ES224 | 5476 | M00085826D:B03 | B-30528 |
| ES224 | 5476 | M00085819C:F06 | B-30528 |
| ES224 | 5476 | M00085266B:C06 | B-30528 |
| ES224 | 5476 | M00086112C:A01 | B-30528 |
| ES224 | 5476 | M00086005A:B02 | B-30528 |
| ES224 | 5476 | M00085548D:F01 | B-30528 |
| ES224 | 5476 | M00085809A:E04 | B-30528 |
| ES224 | 5476 | M00085389B:B05 | B-30528 |
| ES224 | 5476 | M00085761C:E07 | B-30528 |
| ES224 | 5476 | M00085454A:G06 | B-30528 |
| ES224 | 5476 | M00085980B:F06 | B-30528 |
| ES224 | 5476 | M00085367B:C02 | B-30528 |
| ES224 | 5476 | M00085922A:E10 | B-30528 |
| ES224 | 5476 | M00085830B:E09 | B-30528 |
| ES224 | 5476 | M00085611C:D09 | B-30528 |
| ES224 | 5476 | M00085810A:A10 | B-30528 |
| ES224 | 5476 | M00085534D:H09 | B-30528 |
| ES224 | 5476 | M00085390D:A03 | B-30528 |
| ES224 | 5476 | M00085419C:H05 | B-30528 |
| ES224 | 5476 | M00085441D:H10 | B-30528 |
| ES224 | 5476 | M00085434C:G06 | B-30528 |
| ES224 | 5476 | M00085428B:G02 | B-30528 |
| ES224 | 5476 | M00086225B:E01 | B-30528 |
| ES224 | 5476 | M00085835B:E11 | B-30528 |
| ES224 | 5476 | M00085590A:G06 | B-30528 |
| ES224 | 5476 | M00086322D:D05 | B-30528 |
| ES224 | 5476 | M00085255D:E12 | B-30528 |
| ES224 | 5476 | M00086259A:F11 | B-30528 |
| ES224 | 5476 | M00086233C:F01 | B-30528 |
| ES224 | 5476 | M00086038A:D03 | B-30528 |
| ES224 | 5476 | M00085569B:C09 | B-30528 |
| ES224 | 5476 | M00084804C:H10 | B-30528 |
| ES224 | 5476 | M00086000A:C05 | B-30528 |
| ES224 | 5476 | M00085605A:D08 | B-30528 |
| ES224 | 5476 | M00083706A:D02 | B-30528 |
| ES224 | 5476 | M00086202C:A07 | B-30528 |
| ES224 | 5476 | M00083691C:E12 | B-30528 |
| ES224 | 5476 | M00083740C:G01 | B-30528 |
| ES224 | 5476 | M00086159A:F03 | B-30528 |
| ES224 | 5476 | M00085323C:H07 | B-30528 |
| ES224 | 5476 | M00085326B:D08 | B-30528 |
| ES224 | 5476 | M00086270C:D08 | B-30528 |
| ES224 | 5476 | M00086027A:G04 | B-30528 |
| ES224 | 5476 | M00085691A:E06 | B-30528 |
| ES224 | 5476 | M00086276D:F10 | B-30528 |
| ES224 | 5476 | M00086060C:F04 | B-30528 |
| ES224 | 5476 | M00086206A:E10 | B-30528 |
| ES224 | 5476 | M00086087C:D04 | B-30528 |
| ES224 | 5476 | M00083699B:C12 | B-30528 |
| ES224 | 5476 | M00086076B:D02 | B-30528 |
| ES224 | 5476 | M00086288A:B05 | B-30528 |
| ES224 | 5476 | M00085252B:H07 | B-30528 |
| ES224 | 5476 | M00086166D:H04 | B-30528 |
| ES224 | 5476 | M00086184C:D04 | B-30528 |
| ES224 | 5476 | M00085555A:A06 | B-30528 |
| ES224 | 5476 | M00085733D:F08 | B-30528 |
| ES224 | 5476 | M00085887C:B03 | B-30528 |
| ES224 | 5476 | M00086155D:E12 | B-30528 |
| ES224 | 5476 | M00086279C:A08 | B-30528 |
| ES224 | 5476 | M00083710D:B09 | B-30528 |
| ES224 | 5476 | M00086228A:F11 | B-30528 |
| ES224 | 5476 | M00086145A:F07 | B-30528 |
| ES224 | 5476 | M00085100B:C12 | B-30528 |
| ES224 | 5476 | M00085287C:G08 | B-30528 |
| ES224 | 5476 | M00083729C:F10 | B-30528 |
| ES224 | 5476 | M00083692C:D09 | B-30528 |
| ES224 | 5476 | M00086090B:B09 | B-30528 |
| ES224 | 5476 | M00086120A:D11 | B-30528 |
| ES224 | 5476 | M00086031C:G11 | B-30528 |
| ES224 | 5476 | M00085294D:G03 | B-30528 |
| ES224 | 5476 | M00086114D:G11 | B-30528 |
| ES224 | 5476 | M00085720D:A03 | B-30528 |
| ES224 | 5476 | M00085262A:A02 | B-30528 |
| ES224 | 5476 | M00085732A:G04 | B-30528 |
| ES224 | 5476 | M00086159B:E04 | B-30528 |
| ES224 | 5476 | M00084781A:H09 | B-30528 |
| ES224 | 5476 | M00086235A:F05 | B-30528 |
| ES224 | 5476 | M00086097C:B10 | B-30528 |
| ES224 | 5476 | M00084779B:D03 | B-30528 |
| ES224 | 5476 | M00086085D:F09 | B-30528 |
| ES224 | 5476 | M00086286C:H02 | B-30528 |
| ES224 | 5476 | M00083733B:D08 | B-30528 |
| ES224 | 5476 | M00085322D:G05 | B-30528 |
| ES224 | 5476 | M00085071B:D07 | B-30528 |
| ES224 | 5476 | M00085707A:A04 | B-30528 |
| ES224 | 5476 | M00083736A:D10 | B-30528 |
| ES224 | 5476 | M00085301C:H11 | B-30528 |
| ES224 | 5476 | M00085066D:A05 | B-30528 |
| ES224 | 5476 | M00086294D:F08 | B-30528 |
| ES224 | 5476 | M00084773C:D04 | B-30528 |
| ES224 | 5476 | M00085280D:F06 | B-30528 |
| ES224 | 5476 | M00086302A:E06 | B-30528 |
| ES224 | 5476 | M00085084A:E12 | B-30528 |
| ES224 | 5476 | M00085105C:D01 | B-30528 |
| ES224 | 5476 | M00085297A:A11 | B-30528 |
| ES224 | 5476 | M00085076C:H01 | B-30528 |
| ES224 | 5476 | M00086286B:F08 | B-30528 |
| ES224 | 5476 | M00085107D:H08 | B-30528 |
| ES224 | 5476 | M00085092D:D09 | B-30528 |
| ES225 | 5477 | M00086103D:E08 | B-30529 |
| ES225 | 5477 | M00086272D:E04 | B-30529 |
| ES225 | 5477 | M00085449B:G12 | B-30529 |
| ES225 | 5477 | M00085832D:G02 | B-30529 |
| ES225 | 5477 | M00085827C:F03 | B-30529 |
| ES225 | 5477 | M00086328B:G12 | B-30529 |
| ES225 | 5477 | M00085820D:D02 | B-30529 |
| ES225 | 5477 | M00085522C:E05 | B-30529 |
| ES225 | 5477 | M00086178A:D07 | B-30529 |
| ES225 | 5477 | M00085651A:A01 | B-30529 |
| ES225 | 5477 | M00085814B:G08 | B-30529 |
| ES225 | 5477 | M00086149A:F06 | B-30529 |
| ES225 | 5477 | M00085754C:A12 | B-30529 |
| ES225 | 5477 | M00085383C:C05 | B-30529 |
| ES225 | 5477 | M00086206C:C01 | B-30529 |
| ES225 | 5477 | M00085982D:C06 | B-30529 |
| ES225 | 5477 | M00085590B:F11 | B-30529 |
| ES225 | 5477 | M00086161B:H08 | B-30529 |
| ES225 | 5477 | M00086277B:E06 | B-30529 |
| ES225 | 5477 | M00085305B:F10 | B-30529 |
| ES225 | 5477 | M00086081B:A06 | B-30529 |
| ES225 | 5477 | M00086053B:F01 | B-30529 |
| ES225 | 5477 | M00085259A:H05 | B-30529 |
| ES225 | 5477 | M00083694C:F09 | B-30529 |
| ES225 | 5477 | M00085643D:F06 | B-30529 |
| ES225 | 5477 | M00086209B:H12 | B-30529 |
| ES225 | 5477 | M00085860B:D10 | B-30529 |
| ES225 | 5477 | M00086178D:H12 | B-30529 |
| ES225 | 5477 | M00085557B:B02 | B-30529 |
| ES225 | 5477 | M00086184C:C10 | B-30529 |
| ES225 | 5477 | M00086227B:E06 | B-30529 |
| ES225 | 5477 | M00085603A:E01 | B-30529 |
| ES225 | 5477 | M00086233D:A03 | B-30529 |
| ES225 | 5477 | M00085817D:C04 | B-30529 |
| ES225 | 5477 | M00086157D:D03 | B-30529 |
| ES225 | 5477 | M00085583A:E06 | B-30529 |
| ES225 | 5477 | M00086136C:B06 | B-30529 |
| ES225 | 5477 | M00085626B:B11 | B-30529 |
| ES225 | 5477 | M00086048D:H08 | B-30529 |
| ES225 | 5477 | M00086010B:B05 | B-30529 |
| ES225 | 5477 | M00083735C:H12 | B-30529 |
| ES225 | 5477 | M00083722B:A07 | B-30529 |
| ES225 | 5477 | M00085745C:C03 | B-30529 |
| ES225 | 5477 | M00085809A:C05 | B-30529 |
| ES225 | 5477 | M00085694D:D06 | B-30529 |
| ES225 | 5477 | M00085786D:G12 | B-30529 |
| ES225 | 5477 | M00085764B:H12 | B-30529 |
| ES225 | 5477 | M00086000C:B08 | B-30529 |
| ES225 | 5477 | M00085817B:B08 | B-30529 |
| ES225 | 5477 | M00085806B:A10 | B-30529 |
| ES225 | 5477 | M00086081D:D09 | B-30529 |
| ES225 | 5477 | M00085808C:E12 | B-30529 |
| ES225 | 5477 | M00085336C:G09 | B-30529 |
| ES225 | 5477 | M00085620B:D08 | B-30529 |
| ES225 | 5477 | M00085721A:G03 | B-30529 |
| ES225 | 5477 | M00086128D:H10 | B-30529 |
| ES225 | 5477 | M00085361A:A09 | B-30529 |
| ES225 | 5477 | M00085714C:G03 | B-30529 |
| ES225 | 5477 | M00085741C:D06 | B-30529 |
| ES225 | 5477 | M00085722A:A06 | B-30529 |
| ES225 | 5477 | M00083704C:C04 | B-30529 |
| ES225 | 5477 | M00085549D:G03 | B-30529 |
| ES225 | 5477 | M00086143B:B08 | B-30529 |
| ES225 | 5477 | M00085926C:C06 | B-30529 |
| ES225 | 5477 | M00085980A:G10 | B-30529 |
| ES225 | 5477 | M00085625B:F01 | B-30529 |
| ES225 | 5477 | M00086128A:D09 | B-30529 |
| ES225 | 5477 | M00085393D:F12 | B-30529 |
| ES225 | 5477 | M00085935D:H04 | B-30529 |
| ES225 | 5477 | M00086159D:D01 | B-30529 |
| ES225 | 5477 | M00085597C:C03 | B-30529 |
| ES225 | 5477 | M00085259D:B06 | B-30529 |
| ES225 | 5477 | M00086015D:G04 | B-30529 |
| ES225 | 5477 | M00085255D:B09 | B-30529 |
| ES225 | 5477 | M00083715A:B11 | B-30529 |
| ES225 | 5477 | M00085959B:D04 | B-30529 |
| ES225 | 5477 | M00085380B:E10 | B-30529 |
| ES225 | 5477 | M00085100A:A12 | B-30529 |
| ES225 | 5477 | M00085350D:G05 | B-30529 |
| ES225 | 5477 | M00086301A:D04 | B-30529 |
| ES225 | 5477 | M00085891D:E07 | B-30529 |
| ES225 | 5477 | M00085108A:C12 | B-30529 |
| ES225 | 5477 | M00085085C:D10 | B-30529 |
| ES225 | 5477 | M00085104C:A10 | B-30529 |
| ES225 | 5477 | M00084805D:E02 | B-30529 |
| ES225 | 5477 | M00084789D:B10 | B-30529 |
| ES225 | 5477 | M00085277D:C11 | B-30529 |
| ES225 | 5477 | M00085647A:E04 | B-30529 |
| ES225 | 5477 | M00085886C:F05 | B-30529 |
| ES225 | 5477 | M00085444C:C02 | B-30529 |
| ES225 | 5477 | M00085415A:D12 | B-30529 |
| ES225 | 5477 | M00085435A:B11 | B-30529 |
| ES225 | 5477 | M00085282C:F05 | B-30529 |
| ES225 | 5477 | M00084784B:A02 | B-30529 |
| ES225 | 5477 | M00085944A:F04 | B-30529 |
| ES225 | 5477 | M00085804B:F09 | B-30529 |
| ES225 | 5477 | M00085339C:C04 | B-30529 |
| ES225 | 5477 | M00085968B:F09 | B-30529 |
| ES225 | 5477 | M00084779D:H10 | B-30529 |
| ES225 | 5477 | M00085381C:A05 | B-30529 |
| ES225 | 5477 | M00084783C:G08 | B-30529 |
| ES225 | 5477 | M00085333B:B10 | B-30529 |
| ES225 | 5477 | M00085068B:A07 | B-30529 |
| ES225 | 5477 | M00084773C:H08 | B-30529 |
| ES225 | 5477 | M00086020C:E09 | B-30529 |
| ES225 | 5477 | M00085273B:F08 | B-30529 |
| ES225 | 5477 | M00085361D:F06 | B-30529 |
| ES225 | 5477 | M00084780C:F08 | B-30529 |
| ES225 | 5477 | M00085302C:B06 | B-30529 |
| ES225 | 5477 | M00085988D:F09 | B-30529 |
| ES225 | 5477 | M00083738C:D05 | B-30529 |
| ES225 | 5477 | M00085331C:C07 | B-30529 |
| ES225 | 5477 | M00085541C:F06 | B-30529 |
| ES225 | 5477 | M00086327B:D10 | B-30529 |
| ES225 | 5477 | M00085917C:A04 | B-30529 |
| ES225 | 5477 | M00086310A:F04 | B-30529 |
| ES225 | 5477 | M00085510C:A07 | B-30529 |
| ES225 | 5477 | M00085296D:H10 | B-30529 |
| ES225 | 5477 | M00086085A:G05 | B-30529 |
| ES225 | 5477 | M00085299B:G01 | B-30529 |
| ES225 | 5477 | M00085503D:G05 | B-30529 |
| ES225 | 5477 | M00085260C:A10 | B-30529 |
| ES225 | 5477 | M00086282A:B10 | B-30529 |
| ES225 | 5477 | M00085837C:H08 | B-30529 |
| ES225 | 5477 | M00085630B:B09 | B-30529 |
| ES225 | 5477 | M00086279D:C07 | B-30529 |
| ES225 | 5477 | M00085263C:F12 | B-30529 |
| ES225 | 5477 | M00086294B:E11 | B-30529 |
| ES225 | 5477 | M00086322A:E02 | B-30529 |
| ES225 | 5477 | M00085854C:E06 | B-30529 |
| ES225 | 5477 | M00086272D:H11 | B-30529 |
| ES225 | 5477 | M00085422D:D07 | B-30529 |
| ES225 | 5477 | M00085844C:H11 | B-30529 |
| ES225 | 5477 | M00085288C:C09 | B-30529 |
| ES225 | 5477 | M00085082C:B04 | B-30529 |
| ES225 | 5477 | M00085103D:H12 | B-30529 |
| ES225 | 5477 | M00086336B:A08 | B-30529 |
| ES225 | 5477 | M00084773C:F08 | B-30529 |
| ES225 | 5477 | M00085896D:A09 | B-30529 |
| ES225 | 5477 | M00085324B:F10 | B-30529 |
| ES225 | 5477 | M00085267B:D06 | B-30529 |
| ES225 | 5477 | M00085430C:E04 | B-30529 |
| ES225 | 5477 | M00085312C:B09 | B-30529 |
| ES225 | 5477 | M00085074B:A07 | B-30529 |
| ES225 | 5477 | M00085918D:C11 | B-30529 |
| ES225 | 5477 | M00085341C:H08 | B-30529 |
| ES225 | 5477 | M00084791D:D01 | B-30529 |
| ES225 | 5477 | M00085471B:H09 | B-30529 |
| ES225 | 5477 | M00085893B:D08 | B-30529 |
| ES225 | 5477 | M00084781A:A05 | B-30529 |
| ES226 | 5478 | M00084956B:B05 | B-30581 |
| ES226 | 5478 | M00084954D:D12 | B-30581 |
| ES226 | 5478 | M00084948B:F04 | B-30581 |
| ES226 | 5478 | M00084950D:F05 | B-30581 |
| ES226 | 5478 | M00084954D:E01 | B-30581 |
| ES226 | 5478 | M00084941D:C10 | B-30581 |
| ES226 | 5478 | M00084950D:A06 | B-30581 |
| ES226 | 5478 | M00084941D:H02 | B-30581 |
| ES226 | 5478 | M00084954C:B12 | B-30581 |
| ES226 | 5478 | M00084955A:E08 | B-30581 |
| ES226 | 5478 | M00084954D:A05 | B-30581 |
| ES226 | 5478 | M00084951A:D04 | B-30581 |
| ES226 | 5478 | M00084954C:A03 | B-30581 |

Retrieval of Individual Clones from Deposit of Pooled Clones. Where the biological deposit is composed of a pool of cDNA clones or a library of cDNA clones, the deposit was prepared by first transfecting each of the clones into separate bacterial cells. The clones in the pool or library were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

Those skilled in the art will recognize, or be able to ascertain, using not more than routine experimentation, many equivalents to the specific embodiments of the invention described herein. Such specific embodiments and equivalents are intended to be encompassed by the following claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

That which is claimed is:

1. An isolated polynucleotide comprising at least 15 contiguous nucleotides of a sequence selected from the group consisting of SEQ ID NOS:1, 3, 5, 7, 9, 11-13, 15, 16, 18, 20, 22, 24, 26, 27, 29 and 128-1618 and complements thereof.

2. A vector comprising the polynucleotide of claim 1.

3. A host cell comprising the vector of claim 2.

4. An isolated polynucleotide comprising at least 15 contiguous nucleotides of any one of SEQ ID NOS:1, 3, 5, 7, 9, 11-13, 15, 16, 18, 20, 22, 24, 26, 27, 29 and 128-1618 and which hybridizes under stringent conditions to a polynucleotide of a sequence selected from the group consisting of SEQ ID NOS:1, 3, 5, 7, 9, 11-13, 15, 16, 18, 20, 22, 24, 26, 27, 29 and 128-1618 and complements thereof.

5. An isolated polynucleotide comprising at least 15 contiguous nucleotides of either strand of a nucleotide sequence of an insert contained in a vector deposited as clone number XXX-YYY of ATCC Deposit Number ZZZ.

6. An isolated polynucleotide comprising at least 15 contiguous nucleotides of any one of SEQ ID NOS:1, 3, 5, 7, 9, 11-13, 15, 16, 18, 20, 22, 24, 26, 27, 29 and 128-1618, said polynucleotide obtained by amplifying a fragment of cDNA using at least one polynucleotide primer comprising at least 15 contiguous nucleotides of a nucleotide sequence selected from the group consisting of SEQ ID NOS:1, 3, 5, 7, 9, 11-13, 15, 16, 18, 20, 22, 24, 26, 27, 29 and 128-1618 and complements thereof.

7. A method for detecting a cancerous cell, said method comprising:
detecting a level of a gene product corresponding to any one of SEQ ID NOS:1, 3, 5, 7, 9, 11-13, 15, 16, 18, 20, 22, 24, 26, 27, 29 and 128-1618 and complements thereof, and
comparing the level of gene product to a control level of said gene product;
wherein the presence of a cancerous cell is indicated by detection of said level and comparison to a control level of gene product

8. The method of claim 7, wherein said cancerous cell is a cancerous breast, colon or prostate cell.

9. The method of claim 7, wherein said gene product is nucleic acid.

10. The method of claim 7, wherein said gene product is a polypeptide.

11. The method of claim 7, wherein said detecting step uses a polymerase chain reaction.

12. The method of claim 7, wherein said detecting step uses hybridization.

13. The method of claim 7, wherein said sample is a sample of tissue suspected of having cancerous cells.

14. A method for inhibiting a cancerous phenotype of a cell, said method comprising:
contacting a cancerous mammalian cell with an agent for inhibition of a gene product corresponding to any one of SEQ ID NOS:1, 3, 5, 7, 9, 11-13, 15, 16, 18, 20, 22, 24, 26, 27, 29 and 128-1618.

15. The method of claim 14, wherein said cancerous phenotype is aberrant cellular proliferation relative to a normal cell.

16. The method of claim 14, wherein said cancerous phenotype is loss of contact inhibition of cell growth.

17. The method of claims 14, wherein said agent is selected from the group consisting of a small molecule, an antibody, an antisense polynucleotide, and an RNAi molecule.

18. The method of claims 14, wherein said inhibition is associated with a reduction in a level of a gene product corresponding to any one of SEQ ID NOS:1, 3, 5, 7, 9, 11-13, 15, 16, 18, 20, 22, 24, 26, 27, 29 and 128-1618.

19. A method of treating a subject with cancer, said method comprising:
administering to a subject a pharmaceutically effective amount of an agent,
wherein said agent modulates the activity of a gene product corresponding to any one of SEQ ID NOS:113, 5, 7, 9, 11-13, 15, 16, 18, 20, 22, 24, 26, 27, 29 and 128-1618.

20. The method of claim 19, wherein said agent is selected from the group consisting of a small molecule, an antibody, an antisense polynucleotide, and an RNAi molecule.

21. A method for assessing the tumor burden of a subject, said method comprising:
detecting a level of a gene product corresponding to any one of SEQ ID NOS:1, 3, 5, 7, 9, 11-13, 15, 16, 18, 20, 22, 24, 26, 27, 29 and 128-1618 in a test sample from a subject,
wherein the level of said gene product in the test sample is indicative of the tumor burden in the subject.

22. A method for identifying an agent that modulates a biological activity of a gene product differentially expressed in a cancerous cell as compared to a normal cell, said method comprising:
contacting a candidate agent with a cell; and
detecting modulation of a biological activity of a gene product corresponding to any one of SEQ ID NOS:1, 3, 5, 7, 9, 11-13, 15, 16, 18, 20, 22, 24, 26, 27, 29 and 128-1618 relative to a level of biological activity of the same gene product in the absence of the candidate agent.

23. The method of claim 22, wherein said detecting is by assessing expression of said gene product.

24. The method of claim 23, wherein expression is assessed by detecting a polynucleotide gene product.

25. The method of claim 23, wherein expression is assessed by detecting a polypeptide gene product.

26. The method of claim 22, wherein said candidate agent is selected from the group consisting of a small molecule, an antibody, an antisense polynucleotide, and an RNAi molecule.

27. The method of claim 22, wherein said biological activity is modulation of a cancerous phenotype.

28. The method of claim 27, wherein said cancerous phenotype is abnormal cellular proliferation.

29. An isolated polypeptide encoded by any of SEQ ID NOS:1, 3, 5, 7, 9, 11-13, 15, 16, 18, 20, 22, 24, 26, 27, 29 and 128-1618, or fragment or variant thereof.

30. An isolated antibody that specifically binds to a polypeptide encoding by a polynucleotide consisting of a nucleotide sequence set forth in any one of SEQ ID NOS:1, 3, 5, 7, 9, 11-13, 15, 16, 18, 20, 22, 24, 26, 27, 29 and 128-1618 and complements thereof or a polypeptide having an amino acid sequence set forth in SEQ ID NOS: 2, 4, 6, 8, 10, 14, 17, 19, 21, 23, 25, 28 or 1619-1675.

31. An isolated polypeptide comprising at least 6 contiguous amino acids of SEQ ID NOS: 2, 4, 6, 8, 10, 14, 17, 19, 21, 23, 25, 28 or 1619-1675.
